(19) United States
(12) Patent Application Publication
(10) Pub. No.: US 2024/0384268 A1
(43) Pub. Date: Nov. 21, 2024

(54) Oligonucleotides

(71) Applicant: Hudson Institute of Medical Research, Clayton (AU)

(72) Inventor: Michael Paul Marie Gantier, Clayton (AU)

(21) Appl. No.: 18/285,904

(22) PCT Filed: Apr. 8, 2022
(86) PCT No.: PCT/AU2022/050310

§ 371 Date: Oct. 6, 2023

(30) Foreign Application Priority Data
Apr. 8, 2021 (AU) 2021901027
Oct. 27, 2021 (AU) 2021903431

(51) Int. Cl. C12N 15/11; C12N 15/113; C12N 15/117; C12Q 1/6811

(52) U.S. Cl. CPC C12N 15/111; C12N 15/1137; C12N 15/1138; C12N 15/117; C12Q 1/6811; C12N 2310/321; C12N 2310/322; C12N 2310/3231; C12N 2310/341; C12N 2320/11

(57) ABSTRACT

The present disclosure relates to methods of selecting, designing or modifying oligonucleotides so that they inhibit cyclic GMP-AMP synthase (cGAS), Toll-Like Receptor 3 (TLR3,) Toll-Like Receptor 7 (TLR7), Toll-Like Receptor 8 (TLR8) and/or 5 Toll-Like Receptor 9 (TLR9), or potentiate Toll-Like Receptor 8 (TLR8). Additionally, the present disclosure resides in methods of selecting, designing or modifying oligonucleotides such that they exhibit reduced cGAS inhibitory activity.

### CROSS REFERENCE TO RELATED APPLICATIONS

This application claims priority from Australian provisional application nos 2021901027 filed on 8 Apr. 2021 and 2021903431 filed on 27 Oct. 2021, the entire contents of each are incorporated herein by reference in their entirety.

### FIELD OF THE INVENTION

The present invention relates to methods of selecting, designing or modifying oligonucleotides so that they inhibit cyclic GMP-AMP synthase (cGAS), Toll-Like Receptor 3 (TLR3), Toll-Like Receptor 9 (TLR9), Toll-Like Receptor 8 (TLR8), and/or Toll-Like Receptor 7 (TLR7) or potentiate TLR8. Additionally, the present invention resides in methods of selecting, designing or modifying oligonucleotides such that they exhibit reduced cGAS inhibitory activity.

### BACKGROUND OF THE INVENTION

RNA-targeting therapeutics based on synthetic oligonucleotides have been gaining a lot of interest, with several regulatory approvals in the US and European Union (Yin and Rogge, 2019), and multi-billion license deals from big pharma in recent years (Byrne et al., 2020). To ensure their essential functions related to gene targeting activities, oligonucleotides-based therapeutics require both increased affinity for their targets and stabilisation against nuclease activities, through the incorporation of modified nucleotides (e.g., with 2′-O-methyl[2′OMe], 2′-methoxyethyl[2′MOE], 2′-fluoro [2′F], or locked nucleic acid [LNA]) and modified internucleotide linkages (e.g., phosphorothioate [PS]).

The intricate relationship between synthetic oligonucleotides and nucleic acids sensors of the innate immune system, involved in the early detection of pathogens, has been known for two decades. For instance, PS-modified unmethylated “CG” (CpG) containing DNA oligonucleotides have the potential to activate the DNA sensor Toll-Like Receptor (TLR) 9 (Krieg et al., 1995; Hemmi et al., 2000), but can also block it in a sequence and length dependent manner (Krieg et al., 1998; Gursel et al., 2003; Barrat et al., 2005; Trieu et al., 2006). Similarly, 2′OMe modified RNAs block RNA sensing by TLR7 and TLR8 (Robbins et al., 2007; Sioud et al., 2007) and Retinoic Acid Inducible Gene-I (RIG-I) (Devarkar et al., 2016). This knowledge has been important for the design of oligonucleotide therapeutics that can evade activation of innate immune sensors, otherwise leading to strong off-target pro-inflammatory immune responses in patients (Krieg et al., 1995; Judge et al., 2005; Judge et al., 2006). From this angle, chemical modifications can have the dual benefit of increasing the targeting efficacy of the oligonucleotides, while decreasing their immunostimulatory effects.

Nonetheless, it has also been clear for some time that select PS-modified DNA oligonucleotides (ODN) have broad immunosuppressive effects (Bayik et al., 2016). This is best exemplified with the “TTAGGG” containing PS-ODN A151, involved in the inhibition of TLR9 (Gursel et al., 2003), TLR7 (Beignon et al., 2005), Absent In Melanoma 2 (AIM2) (Kaminski et al., 2013) and cyclic-GMP-AMP synthase (cGAS) (Steinhagen et al., 2018). These effects are sequence-dependent, with some PS-DNA ODNs displaying limited immunosuppressive activities on individual immune sensors (Barrat et al., 2005; Bayik et al., 2016). Similarly, 2′OMe oligonucleotides can exhibit sequence-dependent inhibitory effects on TLR7/8 sensing (Sarvestani et al., 2015). These observations suggest a complex picture of immunosuppression by chemically modified oligonucleotides where sequences dictate their activities on nucleic acid sensors. Since only a handful of ODNs have been studied to date across different receptors (Bayik et al., 2016; Steinhagen et al., 2018), a detailed understanding of the immunosuppressive sequence determinants of ODNs is currently lacking. Further, our understanding of the immunosuppressive effects of oligonucleotides combining base and/or backbone modifications, as is seen in most oligonucleotide therapeutics approved and in development, is non-existent. While potentially useful to generate anti-inflammatory ODNs (McWhirter and Jefferies, 2020), characterizing the immunosuppressive effects of therapeutic oligonucleotides is becoming important to help avoid increased susceptibility to infection in the large patient populations who are beginning to receive ODN therapies (Byrne et al., 2020). cGAS has recently emerged as an essential sensor of cytosolic DNA deriving from pathogens and damaged endogenous nucleic acids (McWhirter and Jefferies, 2020). Upon activation by DNA, cGAS drives the formation of cyclic GMP-AMP (cGAMP), which binds to stimulator of interferon genes (STING) and promotes transcriptional induction of IRF3 responsive genes, including CXCL10 (IP-10) and IFNB1. Since it instigates deleterious immune responses linked to a wide range of diseases, various approaches are being investigated currently to therapeutically target cGAS (An et al., 2018; Lama et al., 2019; Padilla-Salinas et al., 2020; Vincent et al., 2017; Zhao et al., 2020). To this end, the majority of drug design strategies have focussed on the development of small molecules inhibiting cGAS enzymatic activity (An et al., 2018; Lama et al., 2019; Padilla-Salinas et al., 2020; Vincent et al., 2017; Zhao et al., 2020; Dai et al., 2019; Hall et al., 2017; Wang et al., 2018), with a propensity to target cGAS systemically rather than in specific tissues. Similar to cGAS, TLR9 is an important factor in autoimmune diseases, and again there is much interest in the development of synthetic TLR9 antagonists that help regulate autoimmune inflammation.

Thus, there is a need for alternative inhibitors of the immunostimulatory effects of cGAS and/or TLR9 activity.

In addition, there is a need for new or improved inhibitors of Toll-Like Receptor 3 (TLR3), Toll-Like Receptor 9 (TLR9), Toll-Like Receptor 8 (TLR8), and/or Toll-Like Receptor 7 (TLR7) activity or new or improved molecules that potentiate TLR8 activity.

### SUMMARY OF THE INVENTION

While designing and testing oligonucleotides, the inventors observed structural features or motifs which assist in inhibiting cGAS, TLR3, TLR7, TLR8 and/or TLR9 activity. The inventors further observed structural features or motifs of these oligonucleotides that assist in maintaining cGAS activity. The inventors further observed structural features or motifs that assist in potentiating TLR8 activity.

Thus, in one aspect, the invention provides a method for selecting or designing an oligonucleotide which inhibits cGAS activity, the method comprising:
i) scanning a polynucleotide, or complement thereof, for a region having a motif including a sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 3)
5′-A[G/A][U/G]C[U/C]C[U/C][C/A]U-3′;
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 7)
5′-CGUUUC-3′;
(SEQ ID NO: 8)
5′-CGUGUC-3′;
(SEQ ID NO: 9)
5′-AUGGCCTTTCCGTGCCAAGG-3′;
(SEQ ID NO: 10)
5′-UCCGGCCTCGGAAGCUCUCU-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 19)
5′-CUUUAGTCGTAGTTGCUUCC-3′;
(SEQ ID NO: 20)
5′-UUAAATAATCTAGTTUGAAG-3′;
(SEQ ID NO: 21)
5′-GUGUCCTTCATGCTTUGGAU-3′;
(SEQ ID NO: 22)
5′-AGAAAGAAGCAAAGAUUCAA-3′;
(SEQ ID NO: 23)
5′-AGAUUATCTTCTTTTAAUUU-3′;
(SEQ ID NO: 24)
5′-AAAAGATTATCTTCTUUUAA-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 27)
5′-CUUGUGAAAAGATTAUCUUC-3′;
(SEQ ID NO: 28)
5′-GGUGGCCACAGGCAACGUCA-3′;
(SEQ ID NO: 29)
5′-CCAUGTCCCAGGCCTCCAGU-3′;
(SEQ ID NO: 30)
5′-GCAGUCTCCATGTCCCAGGC-3′;
(SEQ ID NO: 31)
5′-AGCAGTCTCCATGTCCCAGG-3′;
(SEQ ID NO: 32)
5′-AGUGGCACATACCACACCCU-3′;
(SEQ ID NO: 33)
5′-AUUUCCACATGCCCAGUGUU-3′;
(SEQ ID NO: 34)
5′-GGUCCCATCCCTTCTGCUGC-3′;
(SEQ ID NO: 35)
5′-UCUGGTCCCATCCCTUCUGC-3′;
(SEQ ID NO: 36)
5′-GGGUCTCCTCCACACCCUUC-3′;
(SEQ ID NO: 37)
5′-GCAAGGCAGAGAAACUCCAG-3′;
(SEQ ID NO: 38)
5′-GAUGGTTCCAGTCCCUCUUC-3′;
(SEQ ID NO: 39)
5′-UGUUUCCCCGGAGAGCAAUG-3′;
(SEQ ID NO: 40)
5′-AGCCGAACAGAAGGAGCGUC-3′;
(SEQ ID NO: 41)
5′-GCGUAGTTTCTCTTCCUCCC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 46)
5′-GCCGUTTCCATGTCCCAGGC-3′;
(SEQ ID NO: 47)
5′-GCCGUGTCCATGTCCCAGGC-3′;
(SEQ ID NO: 48)
5′-GCGGUATCCATAGTCUCCAU-3′;
(SEQ ID NO: 49)
5′-GCGGUATCCATCAGAUAUCG-3′;
(SEQ ID NO: 50)
5′-CUUUAGTCGTAGTTGUCUCU-3′;
(SEQ ID NO: 51)
5′-UCCGGGTCGTAGTTGCUUCC-3′;
(SEQ ID NO: 52)
5′-UCCGGCCTCGGAGTCUCCAU-3′;
(SEQ ID NO: 53)
5′-UCCGGCCTCGGGAGAUCUCU-3′;
(SEQ ID NO: 54)
5′-GGUATCCCCCCCCCCCCCCC-3′;
(SEQ ID NO: 55)
5′-GGAUUAAAACAGATTAAUAC-3′;
(SEQ ID NO: 56)
5′-GGUAU-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
(SEQ ID NO: 61)
5′-TGTCTG-3′;
(SEQ ID NO: 62)
5′-GTCT-3′;
(SEQ ID NO: 63)
5′-TCTCCG-3′;
(SEQ ID NO: 64)
5′-CTCC-3′;
(SEQ ID NO: 65)
5′-[G/A][A/C]AG[G/C][T/C]T[C/A];
(SEQ ID NO: 66)
5′-AAAGGTTA-3′;
(SEQ ID NO: 67)
5′-GAAGCTTC-3′;
(SEQ ID NO: 68)
5′-GCAGGCTC-3′;
(SEQ ID NO: 69)
5′-A[G/A]GGTT-3′;
(SEQ ID NO: 70)
5′-AGGGTT-3′;
(SEQ ID NO: 71)
5′-AAGGTT-3′;
(SEQ ID NO: 72)
5′-GGTT-3′;
(SEQ ID NO: 73)
5′-[A/G]GCT[T/C][T/C][G/C][T/A]-3′;
(SEQ ID NO: 74)
5′-AGCTTCCT-3′;
(SEQ ID NO: 75)
5′-AGCTTCGA-3′;
(SEQ ID NO: 76)
5′-GGCTTCGT-3′;
(SEQ ID NO: 77)
5′-TGCTTCCT-3′;
(SEQ ID NO: 78)
5′-AGCTCTCT-3′;
(SEQ ID NO: 79)
5′-G[G/C]TT-3′;
(SEQ ID NO: 80)
5′-GCTT-3′;
(SEQ ID NO: 81)
5′-CGGAGGTCTTGGCTTCGTGG-3′;
(SEQ ID NO: 82)
5′-AGGTCTTGGCTTCGTGGAGC-3′;
(SEQ ID NO: 83)
5′-GGGAAAGGTTATGCAAGGTC-3′;
(SEQ ID NO: 84)
5′-CTGTGATCTTGACATGCTGC-3′;
(SEQ ID NO: 85)
5′-ACTGACTGTCTTGAGGGTTC-3′;
(SEQ ID NO: 86)
5′-GCGTGTCTGGAAGCTTCCTT-3′;
(SEQ ID NO: 87)
5′-GAGTCTCTGGAGCTTCCTCT-3′;
(SEQ ID NO: 88)
5′-AGTCGTAGTTGCTTCCTAAC-3′;
(SEQ ID NO: 89)
5′-GTCTTGGCTTCGTGGAGCAG-3′;
(SEQ ID NO: 90)
5′-TTGGCTCGGCTTGCCTACTT-3′;
(SEQ ID NO: 91)
5′-ACAGTGTTGAGATACTCGGG-3′;
(SEQ ID NO: 92)
5′-TCGCACTTCAGTCTGAGCAG-3′;
(SEQ ID NO: 93)
5′-GGTGTCCTTGCACGTGGCTT-3′;
(SEQ ID NO: 94)
5′-TTTGCACACTTCGTACCCAA-3′;
(SEQ ID NO: 95)
5′-GCTGACAAAGATTCACTGGT-3′;
(SEQ ID NO: 96)
5′-GCGGAGGTCTTGGCTTCGTG-3′;
(SEQ ID NO: 97)
5′-CCAAGATCAGCAGTCT-3′;
(SEQ ID NO: 98)
5′-CTTGAAGCATCGTATC-3′;
(SEQ ID NO: 99)
5′-GCACACTTCGTACCCA-3′;
(SEQ ID NO: 100)
5′-GATAGCACCTTCAGCA-3′;
(SEQ ID NO: 101)
5′-CGTATTATAGCCGATT-3′;
(SEQ ID NO: 102)
5′-GCAGGCTCAGTGATGT-3′;
(SEQ ID NO: 103)
5′-GAAAGGTTATGCAAGG-3′;
(SEQ ID NO: 104)
5′-ATGGCCTCCCATCTCC-3′;
(SEQ ID NO: 105)
5′-CGCTTTTCTGTCTGGT-3′;
(SEQ ID NO: 106)
5′-GTGTCTGGAAGCTTCC-3′;
(SEQ ID NO: 107)
5′-TGGCCTCCCATCTCCT-3′;
(SEQ ID NO: 108)
5′-ATCTGGCAGCCCATCA-3′;
(SEQ ID NO: 109)
5′-GAGGTCTTGGCTTCGT-3′;
(SEQ ID NO: 110)
5′-ACACTTCGTGGGGTCC-3′;
(SEQ ID NO: 111)
5′-TTCGTGGGGTCCTTTT-3′;
(SEQ ID NO: 112)
5′-CCACTTGGCAGACCAT-3′;
(SEQ ID NO: 113)
5′-CCATCCATGAGGTCCT-3′;
(SEQ ID NO: 114)
5′-TCCAACACTTCGTGGG-3′;
(SEQ ID NO: 115)
5′-TCTTCATCGGCCCTGC-3′;
(SEQ ID NO: 116)
5′-CCAGCAGGTCAGCAAA-3′;
(SEQ ID NO: 117)
5′-CGCTTTTCTCTCCGGT-3′;
(SEQ ID NO: 118)
5′-GGUAUAGGACTCCAGATGUUUCC-3′;

a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit cGAS activity, and
iv) selecting an oligonucleotide which inhibits cGAS activity.

In an embodiment, step i) includes scanning a polynucleotide, or complement thereof, for the motif with the sequence of 5′-[A/G]GU[A/C][U/C]C-3′ (SEQ ID NO: 1), 5′-A[G/A][U/G]C[U/C]C-3′ (SEQ ID NO: 2) or 5′-A[G/A][U/G]C[U/C]C[U/C][C/A]U-3′ (SEQ ID NO: 3), wherein the U may be a T and/or the T may be a U. Suitably, the motif of 5′-[A/G]GU[A/C][U/C]C-3′ (SEQ ID NO: 1), 5′-A[G/A][U/G]C[U/C]C-3′ (SEQ ID NO: 2) or 5′-A[G/A][U/G]C[U/C]C[U/C][C/A]U-3′ (SEQ ID NO: 3) is at or towards a 5′ end of the oligonucleotide.

In another embodiment, step i) includes scanning a polynucleotide, or complement thereof, for the motif with the sequence of 5′-GGUAUA-3′ (SEQ ID NO: 4) or a variant having at least about 75% sequence identity thereto, wherein the U may be a T and/or the T may be a U. Suitably, the motif of 5′-GGUAUA-3′ (SEQ ID NO: 4) is at or towards a 5′ end of the oligonucleotide.

In still a further embodiment, step i) includes scanning a polynucleotide, or complement thereof, for the motif with the sequence of 5′-GGUAU-3′ (SEQ ID NO: 56), 5′-GGUA-3 (SEQ ID NO: 57)′, 5′-GUAU-3′ (SEQ ID NO: 58), 5′-GGU-3′ (SEQ ID NO: 59) or 5′-GUA-3′ (SEQ ID NO: 60) or a variant having at least about 75% sequence identity thereto, wherein the U may be a T and/or the T may be a U. Suitably, the motif of 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60) is at or towards a 5′ end of the oligonucleotide.

In a related aspect, the invention provides a method for increasing the cGAS inhibitory activity of an oligonucleotide, the method comprising modifying the oligonucleotide such that the modified oligonucleotide comprises a motif with a sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 3)
5′-A[G/A][U/G]C[U/C]C[U/C][C/A]U-3′;
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 7)
5′-CGUUUC-3′;
(SEQ ID NO: 8)
5′-CGUGUC-3′;
(SEQ ID NO: 9)
5′-AUGGCCTTTCCGTGCCAAGG-3′;
(SEQ ID NO: 10)
5′-UCCGGCCTCGGAAGCUCUCU-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 19)
5′-CUUUAGTCGTAGTTGCUUCC-3′;
(SEQ ID NO: 20)
5′-UUAAATAATCTAGTTUGAAG-3′;
(SEQ ID NO: 21)
5′-GUGUCCTTCATGCTTUGGAU-3′;
(SEQ ID NO: 22)
5′-AGAAAGAAGCAAAGAUUCAA-3′;
(SEQ ID NO: 23)
5′-AGAUUATCTTCTTTTAAUUU-3′;
(SEQ ID NO: 24)
5′-AAAAGATTATCTTCTUUUAA-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 27)
5′-CUUGUGAAAAGATTAUCUUC-3′;
(SEQ ID NO: 28)
5′-GGUGGCCACAGGCAACGUCA-3′;
(SEQ ID NO: 29)
5′-CCAUGTCCCAGGCCTCCAGU-3′;
(SEQ ID NO: 30)
5′-GCAGUCTCCATGTCCCAGGC-3′;
(SEQ ID NO: 31)
5′-AGCAGTCTCCATGTCCCAGG-3′;
(SEQ ID NO: 32)
5′-AGUGGCACATACCACACCCU-3′;
(SEQ ID NO: 33)
5′-AUUUCCACATGCCCAGUGUU-3′;
(SEQ ID NO: 34)
5′-GGUCCCATCCCTTCTGCUGC-3′;
(SEQ ID NO: 35)
5′-UCUGGTCCCATCCCTUCUGC-3′;
(SEQ ID NO: 36)
5′-GGGUCTCCTCCACACCCUUC-3′;
(SEQ ID NO: 37)
5′-GCAAGGCAGAGAAACUCCAG-3′;
(SEQ ID NO: 38)
5′-GAUGGTTCCAGTCCCUCUUC-3′;
(SEQ ID NO: 39)
5′-UGUUUCCCCGGAGAGCAAUG-3′;
(SEQ ID NO: 40)
5′-AGCCGAACAGAAGGAGCGUC-3′;
(SEQ ID NO: 41)
5′-GCGUAGTTTCTCTTCCUCCC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 46)
5′-GCCGUTTCCATGTCCCAGGC-3′;
(SEQ ID NO: 47)
5′-GCCGUGTCCATGTCCCAGGC-3′;
(SEQ ID NO: 48)
5′-GCGGUATCCATAGTCUCCAU-3′;
(SEQ ID NO: 49)
5′-GCGGUATCCATCAGAUAUCG-3′;
(SEQ ID NO: 50)
5′-CUUUAGTCGTAGTTGUCUCU-3′;
(SEQ ID NO: 51)
5′-UCCGGGTCGTAGTTGCUUCC-3′;
(SEQ ID NO: 52)
5′-UCCGGCCTCGGAGTCUCCAU-3′;
(SEQ ID NO: 53)
5′-UCCGGCCTCGGGAGAUCUCU-3′;
(SEQ ID NO: 54)
5′-GGUATCCCCCCCCCCCCCCC-3′;
(SEQ ID NO: 55)
5′-GGAUUAAAACAGATTAAUAC-3′;
(SEQ ID NO: 56)
5′-GGUAU-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
(SEQ ID NO: 61)
5′-TGTCTG-3′;
(SEQ ID NO: 62)
5′-GTCT-3′;
(SEQ ID NO: 63)
5′-TCTCCG-3′;
(SEQ ID NO: 64)
5′-CTCC-3′;
(SEQ ID NO: 65)
5′-[G/A][A/C]AG[G/C][T/C]T[C/A];
(SEQ ID NO: 66)
5′-AAAGGTTA-3′;
(SEQ ID NO: 67)
5′-GAAGCTTC-3′;
(SEQ ID NO: 68)
5′-GCAGGCTC-3′;
(SEQ ID NO: 69)
5′-A[G/A]GGTT-3′;
(SEQ ID NO: 70)
5′-AGGGTT-3′;
(SEQ ID NO: 71)
5′-AAGGTT-3′;
(SEQ ID NO: 72)
5′-GGTT-3′;
(SEQ ID NO: 73)
5′-[A/G]GCT[T/C][T/C][G/C][T/A]-3′;
(SEQ ID NO: 74)
5′-AGCTTCCT-3′;
(SEQ ID NO: 75)
5′-AGCTTCGA-3′;
(SEQ ID NO: 76)
5′-GGCTTCGT-3′;
(SEQ ID NO: 77)
5′-TGCTTCCT-3′;
(SEQ ID NO: 78)
5′-AGCTCTCT-3′;
(SEQ ID NO: 79)
5′-G[G/C]TT-3′;
(SEQ ID NO: 80)
5′-GCTT-3′;
(SEQ ID NO: 81)
5′-CGGAGGTCTTGGCTTCGTGG-3′;
(SEQ ID NO: 82)
5′-AGGTCTTGGCTTCGTGGAGC-3′;
(SEQ ID NO: 83)
5′-GGGAAAGGTTATGCAAGGTC-3′;
(SEQ ID NO: 84)
5′-CTGTGATCTTGACATGCTGC-3′;
(SEQ ID NO: 85)
5′-ACTGACTGTCTTGAGGGTTC-3′;
(SEQ ID NO: 86)
5′-GCGTGTCTGGAAGCTTCCTT-3′;
(SEQ ID NO: 87)
5′-GAGTCTCTGGAGCTTCCTCT-3′;
(SEQ ID NO: 88)
5′-AGTCGTAGTTGCTTCCTAAC-3′;
(SEQ ID NO: 89)
5′-GTCTTGGCTTCGTGGAGCAG-3′;
(SEQ ID NO: 90)
5′-TTGGCTCGGCTTGCCTACTT-3′;
(SEQ ID NO: 91)
5′-ACAGTGTTGAGATACTCGGG-3′;
(SEQ ID NO: 92)
5′-TCGCACTTCAGTCTGAGCAG-3′;
(SEQ ID NO: 93)
5′-GGTGTCCTTGCACGTGGCTT-3′;
(SEQ ID NO: 94)
5′-TTTGCACACTTCGTACCCAA-3′;
(SEQ ID NO: 95)
5′-GCTGACAAAGATTCACTGGT-3′;
(SEQ ID NO: 96)
5′-GCGGAGGTCTTGGCTTCGTG-3′;
(SEQ ID NO: 97)
5′-CCAAGATCAGCAGTCT-3′;
(SEQ ID NO: 98)
5′-CTTGAAGCATCGTATC-3′;
(SEQ ID NO: 99)
5′-GCACACTTCGTACCCA-3′;
(SEQ ID NO: 100)
5′-GATAGCACCTTCAGCA-3′;
(SEQ ID NO: 101)
5′-CGTATTATAGCCGATT-3′;
(SEQ ID NO: 102)
5′-GCAGGCTCAGTGATGT-3′;
(SEQ ID NO: 103)
5′-GAAAGGTTATGCAAGG-3′;
(SEQ ID NO: 104)
5′-ATGGCCTCCCATCTCC-3′;
(SEQ ID NO: 105)
5′-CGCTTTTCTGTCTGGT-3′;
(SEQ ID NO: 106)
5′-GTGTCTGGAAGCTTCC-3′;
(SEQ ID NO: 107)
5′-TGGCCTCCCATCTCCT-3′;
(SEQ ID NO: 108)
5′-ATCTGGCAGCCCATCA-3′;
(SEQ ID NO: 109)
5′-GAGGTCTTGGCTTCGT-3′;
(SEQ ID NO: 110)
5′-ACACTTCGTGGGGTCC-3′;
(SEQ ID NO: 111)
5′-TTCGTGGGGTCCTTTT-3′;
(SEQ ID NO: 112)
5′-CCACTTGGCAGACCAT-3′;
(SEQ ID NO: 113)
5′-CCATCCATGAGGTCCT-3′;
(SEQ ID NO: 114)
5′-TCCAACACTTCGTGGG-3′;
(SEQ ID NO: 115)
5′-TCTTCATCGGCCCTGC-3′;
(SEQ ID NO: 116)
5′-CCAGCAGGTCAGCAAA-3′;
(SEQ ID NO: 117)
5′-CGCTTTTCTCTCCGGT-3′;
(SEQ ID NO: 118)
5′-GGUAUAGGACTCCAGATGUUUCC-3′;

a variant of the motifs or sequences thereof having at least about 75% sequence identity thereto;
wherein the U may be a T and/or the T may be a U.

In an embodiment, the step of modifying the oligonucleotide includes adding a sequence of nucleotides to the 5′ and/or 3′ end of the oligonucleotide such that the modified oligonucleotide comprises the motif.

In one particular embodiment, the step of modifying the oligonucleotide includes adding a sequence of nucleotides, such as 5′-GGUATC-3′ (SEQ ID NO: 119), 5′-GGUAUC-3′ (SEQ ID NO: 120) or a fragment or portion thereof, to the 5′ end of the oligonucleotide such that the modified oligonucleotide comprises the motif.

In some embodiments, the step of modifying the oligonucleotide includes adding the motif of 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60) or a fragment or portion thereof, wherein the U may be a T, to the 5′ and/or 3′ end of the oligonucleotide, such that the modified oligonucleotide comprises the motif More particularly, the step of modifying the oligonucleotide suitably includes adding 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60) or a fragment or portion thereof, wherein the U may be a T, to the 5′ end of the oligonucleotide, such that the modified oligonucleotide comprises the motif.

In an embodiment, the method of the present aspect further comprises testing the ability of the modified oligonucleotide to inhibit cGAS activity, and selecting an oligonucleotide which inhibits cGAS activity to a greater extent than the unmodified oligonucleotide.

In an embodiment of the above aspects, the oligonucleotide does not bind or is not designed to bind a transcript that encodes cGAS or a complement thereof.

In another embodiment of the above aspects, the oligonucleotide binds or is designed to bind a target transcript that does not encode cGAS or a complement thereof.

In an alternative embodiment of the above aspects, the oligonucleotide binds or is designed to bind a target transcript that encodes cGAS or a complement thereof.

In yet another embodiment, the oligonucleotide does not bind or is not designed to bind a target transcript.

In an embodiment of the above aspects, the motif is within eleven bases of the 5′ and/or 3′ end of the oligonucleotide.

In a further embodiment of the above aspects, the motif is within eight bases of the 5′ and/or 3′ end of the oligonucleotide.

In yet a further embodiment of the above aspects, the motif is at or towards the 5′ and/or 3′ end of the oligonucleotide. By way of example, in embodiments in which the motif is 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60), wherein the U may be a T, the motif is suitably at or towards the 5′ end of the oligonucleotide.

Examples of the motif of the above aspects include, but are not limited to, those having the sequence 5′-GGUAUC-3′ (SEQ ID NO: 120), 5′-AGUCUC-3′ (SEQ ID NO: 121), 5′-GGUCCC-3′ (SEQ ID NO: 122), 5′-GGUCUC-3′ (SEQ ID NO: 123), 5′-AAGCUC-3′ (SEQ ID NO: 124), 5′-AGUCCC-3′ (SEQ ID NO: 125), 5′-GGUAUA-3′ (SEQ ID NO: 4), 5′-UGUUUC-3′ (SEQ ID NO: 5), 5′-UGUGUC-3′ (SEQ ID NO: 6), 5′-CGUUUC-3′ (SEQ ID NO: 7), 5′-CGUGUC-3′ (SEQ ID NO: 8), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60), 5′-TGTCTG-3′ (SEQ ID NO: 61), 5′-GTCT-3′ (SEQ ID NO: 62), 5′-TCTCCG-3′ (SEQ ID NO: 63), 5′-CTCC-3′ (SEQ ID NO: 64), 5′-AAAGGTTA-3′ (SEQ ID NO: 66), 5′-GAAGCTTC-3′ (SEQ ID NO: 67), 5′-GCAGGCTC-3′ (SEQ ID NO: 68), 5′-AGGGTT-3′ (SEQ ID NO: 70), 5′-AAGGTT-3′ (SEQ ID NO: 71), 5′-GGTT-3′ (SEQ ID NO: 72), 5′-AGCTTCCT-3′ SEQ ID NO: 74), 5′-AGCTTCGA-3′ (SEQ ID NO: 75), 5′-GGCTTCGT-3′ (SEQ ID NO: 76), 5′-TGCTTCCT-3′ (SEQ ID NO: 77), 5′-AGCTCTCT-3′ (SEQ ID NO: 78) or 5′-GCTT-3′ (SEQ ID NO: 80), wherein the U may be a T. More particularly, the motif of the above aspects suitably has the sequence of 5′-GGUAUC-3′ (SEQ ID NO: 120), 5′-GGUATC-3′ (SEQ ID NO: 119), 5′-AGUCTC-3′ (SEQ ID NO: 126), 5′-AGTCTC-3′ (SEQ ID NO: 127), 5′-GGUCCC-3′ (SEQ ID NO: 122), 5′-GGUCTC-3′ (SEQ ID NO: 128), 5′-AAGCUC-3′ (SEQ ID NO: 124), 5′-AGTCCC-3′ (SEQ ID NO: 129), 5′-GGUATA-3′ (SEQ ID NO: 130), 5′-UGUTTC-3′ (SEQ ID NO: 131), 5′-UGUGTC-3′ (SEQ ID NO: 132), 5′-CGUTTC-3′ (SEQ ID NO: 133), 5′-CGUGTC-3′ (SEQ ID NO: 134), 5′-GGUAU-3′ (SEQ ID NO: 56), 5′-GGUAT-3′ (SEQ ID NO: 135), 5′-GGUA-3′ (SEQ ID NO: 57), 5′-GUAU-3′ (SEQ ID NO: 58), 5′-GUAT-3′ (SEQ ID NO: 136), 5′-GGU-3′ (SEQ ID NO: 59), 5′-GUA-3′ (SEQ ID NO: 60), 5′-TGTCTG-3′ (SEQ ID NO: 61), 5′-GTCT-3′ (SEQ ID NO: 62), 5′-TCTCCG-3′ (SEQ ID NO: 63), 5′-CTCC-3′ (SEQ ID NO: 64), 5′-AAAGGTTA-3′ (SEQ ID NO: 66), 5′-GAAGCTTC-3′ (SEQ ID NO: 67), 5′-GCAGGCTC-3′ (SEQ ID NO: 68), 5′-AGGGTT-3′ (SEQ ID NO: 70), 5′-AAGGTT-3′ (SEQ ID NO: 71), 5′-GGTT-3′ (SEQ ID NO: 72), 5′-AGCTTCCT-3′ (SEQ ID NO: 74), 5′-AGCTTCGA-3′ (SEQ ID NO: 75), 5′-GGCTTCGT-3′ (SEQ ID NO: 76), 5′-TGCTTCCT-3′ (SEQ ID NO: 77), 5′-AGCTCTCT-3′ (SEQ ID NO: 78) or 5′-GCTT-3′ (SEQ ID NO: 80).

In one particular embodiment, the motif of the above aspects has the sequence of 5′-GGUAUC-3′ (SEQ ID NO: 120), 5′-GGUATC-3′ (SEQ ID NO: 119), 5′-GCGGUATCCATGTCCCAGGC-3′ (SEQ ID NO: 42), 5′-GCGGUAUCCATGTCCCAGGC-3′ (SEQ ID NO: 137), 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60) or a variant thereof having at least about 75% sequence identity thereto, wherein the U may be a T and/or the T may be a U.

In an embodiment, one or more of the bases of the motif of the above aspects are a modified base and/or have a modified backbone.

In particular embodiments, the motif of the above aspects has the sequence of 5′-mGmGmUATC-3′, 5′-mGmGmUAUC-3′, 5′-mAmGmUCTC-3′, 5′-mAmGTCTC-3′, 5′-mGmGmUmCmCC-3′, 5′-mGmGmUmCTC-3′, 5′-AAGCmUmC-3′, 5′-AGTCCC-3′ (SEQ ID NO: 129), 5′-mGmGmUATA-3′, 5′-mUmGmUTTC-3′, 5′-mUmGmUGTC-3′, 5′-mCmGmUTTC-3′, 5′-mCmGmUGTC-3′, 5′-mGmGmUAU-3′, 5′-mGmGmUAT-3′, 5′-mGmGmUmAU-3′, 5′-mGmGmUmAT-3′, 5′-mGmGmUmAmU-3′, 5′-mGmGmUA-3′, 5′-mGmGmUmA-3′, 5′-mGmGmU-3′, 5′-mTmGTCTG-3′, 5′-TGTCTmG-3′, mTmGTCTG-3′, 5′-mGTCT-3′, 5′-GTCT-3′ (SEQ ID NO: 62), 5′-TCTCCG-3′ (SEQ ID NO: 63), 5′-TCTCCmG-3′, 5′-CTCC-3′ (SEQ ID NO: 64), 5′-mAmAAGGTTA-3′, 5′-GAAGCTmTmC-3′, 5′-mGmCmAGGCTC-3′, 5′-mAAGGTT-3′, 5′-AGmGmGmTmT-3′, 5′-AGCTmTmCmCmT-3′, 5′-AGCTTmCmCmT-3′, 5′-mAmGmCTTCGA-3′, 5′-GGCTTmCmGmT-3′, 5′-GGCTTCGT-3′ (SEQ ID NO: 76), 5′-TGCTTCmCmT-3′ or 5′-AGCmTmCmTmCmT-3′, wherein m is a modified base and/or has a modified backbone.

In one particular embodiment, the motif of the above aspects has the sequence of:

5′-mGmGmUATC-3′;
5′-mGmGmUAUC-3′;
5′-mGmCmGmGmUATCCATGTCCmCmAmGmGmC-3′;
5′-mGmCmGmGmUAUCCATGTCCmCmAmGmGmC-3′;
5′-mGmGmUmAmUmC-3′;
5′-mGmCmGmGmUmAmUmCmCmAmUmGmUmCmCmCmAmGmGmC-3′;
5′-mGmGmUATCCCCCCCCCCCCCCC-3′;
5′-mGmGmUAU-3′;
5′-mGmGmUAT-3′;
5′-mGmGmUmAU-3′;
5′-mGmGmUmAT-3′;
5′-mGmGmUmAmU-3′;
5′-mGmGmUA-3′;
5′-mGmGmUmA-3′;
5′-mGmUmAmU-3′;
5′-mGmGmU-3′;
5′-mGmUmA-3′;

or a variant thereof having at least about 75% sequence identity thereto, wherein the U may be a T and/or the T may be a U and wherein m is a modified base and/or has a modified backbone.

In one embodiment of the above two aspects, the motif has the sequence: 5′-CGCTTTTCTGTCTGGT-3′ (SEQ ID NO: 105); 5′-GAAAGGTTATGCAAGG-3′ (SEQ ID NO: 103); 5′-GCAGGCTCAGTGATGT-3′ (SEQ ID NO: 102); or 5′-GTGTCTGGAAGCTTCC-3′ (SEQ ID NO: 106), wherein the U may be a T and/or the T may be a U.

In particular embodiments of the above two aspects, the motif has the sequence:

5′-mCmGmCTTTTCTGTCTmGmGmT-3′;
5′-mGmAmAAGGTTATGCAmAmGmG-3′;
5′-mGmCmAGGCTCAGTGAmTmGmT-3′;
or
5′-mGmTmGTCTGGAAGCTmTmCmC-3′;

wherein the U may be a T and/or the T may be a U and wherein m is a modified base and/or has a modified backbone. For such examples, m is suitably a 2′-LNA modified base.

In another embodiment of the above two aspects, the motif has the sequence:

(SEQ ID NO: 81)
5′-CGGAGGTCTTGGCTTCGTGG-3′;
(SEQ ID NO: 14)
5′-GGAGCTTCGAGGCCCCAGGC-3′;
(SEQ ID NO: 82)
5′-AGGTCTTGGCTTCGTGGAGC-3′;
(SEQ ID NO: 83)
5′-GGGAAAGGTTATGCAAGGTC-3′;
(SEQ ID NO: 84)
5′-CTGTGATCTTGACATGCTGC-3′;
(SEQ ID NO: 85)
5′-ACTGACTGTCTTGAGGGTTC-3′;
(SEQ ID NO: 86)
5′-GCGTGTCTGGAAGCTTCCTT-3′;
(SEQ ID NO: 138)
5′-TCCGGCCTCGGAAGCTCTCT-3′;
(SEQ ID NO: 87)
5′-GAGTCTCTGGAGCTTCCTCT-3′;
(SEQ ID NO: 139)
5′-GGTCTTGGCTTCGTGGAGCA-3′;
or
(SEQ ID NO: 88)
5′-AGTCGTAGTTGCTTCCTAAC-3′;

wherein the U may be a T and/or the T may be a U.

In articular embodiments of the above two aspects, the motif has the sequence:

5′-mCmGmGmAmGGTCTTGGCTTmCmGmTmGmG-3′;
5′-mGmGmAmGmCTTCGAGGCCCmCmAmGmGmC-3′;
5′-mAmGmGmTmCTTGGCTTCGTmGmGmAmGmC-3′;
5′-mGmGmGmAmAAGGTTATGCAmAmGmGmTmC-3′;
5′-mCmTmGmTmGATCTTGACATmGmCmTmGmC-3′;
5′-mAmCmTmGmACTGTCTTGAGmGmGmTmTmC-3′;
5′-mGmCmGmTmGTCTGGAAGCTmTmCmCmTmT-3′;
5′-mTmCmCmGmGCCTCGGAAGCmTmCmTmCmT-3′;
5′-mGmAmGmTmCTCTGGAGCTTmCmCmTmCmT-3′;
5′-mGmGmTmCmTTGGCTTCGTGmGmAmGmCmA-3′;
or
5′-mAmGmTmCmGTAGTTGCTTCmCmTmAmAmC-3′;

wherein the U may be a T and/or the T may be a U and wherein m is a modified base and/or has a modified backbone. For such examples, m is suitably a 2′-MOE modified base.

In another aspect, the invention provides a method for selecting or designing an oligonucleotide which does not inhibit cGAS activity, the method comprising
i) scanning a polynucleotide, or complement thereof, for a region having a motif including a sequence selected from the group consisting of:

(SEQ ID NO: 140)
5′-[C/U]CUUCU-3′;
(SEQ ID NO: 141)
5′-CACCCTTCTCTCTGGUCCCA-3′;
(SEQ ID NO: 142)
5′-CCUUCTCTCTGGTCCCAUCC-3′;
(SEQ ID NO: 143)
5′-UCUCUGGTCCCATCCCUUCU-3′;
(SEQ ID NO: 144)
5′-AUAUCTGCTGCCCACCUUCU-3′;
(SEQ ID NO: 145)
5′-GUCCCATCCCTTCTGCUGCC-3′;
(SEQ ID NO: 146)
5′-GUCUCCTCCACACCCUUCUC-3′;
(SEQ ID NO: 147)
5′-CAGGCCTCCAGTGTCUUCUC-3′;
(SEQ ID NO: 148)
5′-UCCCAACTCTTCTAACUCGU-3′;

a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit cGAS activity, and
iv) selecting an oligonucleotide which does not inhibit cGAS activity.

In a related aspect, the invention resides in a method for reducing the cGAS inhibitory activity of an oligonucleotide, the method comprising modifying the oligonucleotide such that the modified oligonucleotide comprises a motif having a sequence selected from the group consisting of:

(SEQ ID NO: 140)
5′-[C/U]CUUCU-3′;
(SEQ ID NO: 141)
5′-CACCCTTCTCTCTGGUCCCA-3′;
(SEQ ID NO: 142)
5′-CCUUCTCTCTGGTCCCAUCC-3′;
(SEQ ID NO: 143)
5′-UCUCUGGTCCCATCCCUUCU-3′;
(SEQ ID NO: 144)
5′-AUAUCTGCTGCCCACCUUCU-3′;
(SEQ ID NO: 145)
5′-GUCCCATCCCTTCTGCUGCC-3′;
(SEQ ID NO: 146)
5′-GUCUCCTCCACACCCUUCUC-3′;
(SEQ ID NO: 147)
5′-CAGGCCTCCAGTGTCUUCUC-3′;
(SEQ ID NO: 148)
5′-UCCCAACTCTTCTAACUCGU-3′;

a variant of the motifs or sequences thereof having at least about 75% sequence identity thereto;
wherein the U may be a T and/or the T may be a U.

In an embodiment, the step of modifying the oligonucleotide includes adding a sequence of nucleotides to the 5′ and/or 3′ end of the oligonucleotide such that the modified oligonucleotide comprises the motif.

In an embodiment, the present method further comprises testing the ability of the modified oligonucleotide to inhibit cGAS activity, and selecting an oligonucleotide which inhibits cGAS activity to a lesser extent than the unmodified oligonucleotide.

Referring to the two aforementioned aspects, the motif is suitably within thirteen bases of the 5′ and/or 3′ end of the oligonucleotide. More particularly, the motif is suitably within nine bases of the 5′ and/or 3′ end of the oligonucleotide. Even more particularly, the motif is suitably at or towards the 5′ and/or 3′ end of the oligonucleotide.

In an embodiment of the two aforementioned aspects, the motif has the sequence 5′-CCUUCU-3′ (SEQ ID NO: 149) or 5′-UCUUCU-3′ (SEQ ID NO: 150), wherein the U may be a T.

In an embodiment of the two aforementioned aspects, one or more of the bases of the motif are a modified base and/or have a modified backbone.

In an embodiment of the two aforementioned aspects, the motif has the sequence 5′-mCmCUUCU-3′, 5′-mCmCmUmUmCU-3′, 5′-CmCmUmUmCmU-3′, 5′-CCUUCU-3′ (SEQ ID NO: 149), 5′-CCmUmUmCmU-3′, 5′-UCmUmUmCmU-3′, 5′-UCUUCU-3′ (SEQ ID NO: 150), 5′-UmCmUmUmCmU-3′ or 5′-CmCmUmUmCmU-3′ wherein the U may be a T and wherein m is a modified base and/or has a modified backbone.

In any embodiment of the above aspects, the motif comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of cGAS.

With respect to the above aspects, the method may include the further step of testing the ability of the one or more candidate oligonucleotides or the modified oligonucleotide to inhibit TLR3, TLR7 and/or TLR9 activity, and optionally selecting an oligonucleotide which inhibits or does not substantially inhibit TLR3, TLR7 and/or TLR9 activity.

In particular embodiments, the one or more candidate oligonucleotides or the modified oligonucleotide inhibit cGAS and TLR7 activity. Examples of the motif of such embodiments include 5′-GCAGUCTCCATGTCCCAGGC-3′ (SEQ ID NO: 30), 5′-GAUGGTTCCAGTCCCUCUUC-3′ (SEQ ID NO: 38), 5′-AGCAGTCTCCATGTCCCAGG-3′ (SEQ ID NO: 31), 5′-GGGUCTCCTCCACACCCUUC-3′ (SEQ ID NO: 36), 5′-GGUGGCCACAGGCAACGUCA-3′ (SEQ ID NO: 28), 5′-GCCGUTTCCATGTCCCAGGC-3′ (SEQ ID NO: 46), 5′-GCGGUATCCATGTCCCAGGC-3′ (SEQ ID NO: 42), 5′-GCGGUATACAGGTCCCAGGC-3′ (SEQ ID NO: 43), 5′-GCUGUTTCCATGTCCCAGGC-3′ (SEQ ID NO: 44), 5′-GCUGUGTCCATGTCCCAGGC-3′ (SEQ ID NO: 45), 5′-GCCGUGTCCATGTCCCAGGC-3′ (SEQ ID NO: 47), 5′-GCGGUAUCCAUGUCCCAGGC-3′ (SEQ ID NO: 151), 5′-GGUATCCCCCCCCCCCCCCC-3′ (SEQ ID NO: 54), 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In further embodiments, the one or more candidate oligonucleotides or the modified oligonucleotide inhibit cGAS and TLR7 activity, but do not substantially inhibit TLR9 activity. Examples of the motif of such embodiments include 5′-GCAGUCTCCATGTCCCAGGC-3′ (SEQ ID NO: 30), 5′-GAUGGTTCCAGTCCCUCUUC-3′ (SEQ ID NO: 38), 5′-AGCAGTCTCCATGTCCCAGG-3′ (SEQ ID NO: 31), 5′-GGGUCTCCTCCACACCCUUC-3′ (SEQ ID NO: 36), 5′-GGUGGCCACAGGCAACGUCA-3′ (SEQ ID NO: 28), 5′-GCCGUTTCCATGTCCCAGGC-3′ (SEQ ID NO: 46), 5′-GCGGUATCCATGTCCCAGGC-3′ (SEQ ID NO: 42), 5′-GCGGUATACAGGTCCCAGGC-3′ (SEQ ID NO: 43), 5′-GCUGUTTCCATGTCCCAGGC-3′ (SEQ ID NO: 44), 5′-GCUGUGTCCATGTCCCAGGC-3′ (SEQ ID NO: 45), 5′-GCCGUGTCCATGTCCCAGGC-3′ (SEQ ID NO: 47), 5′-GCGGUAUCCAUGUCCCAGGC-3′ (SEQ ID NO: 151) and 5′-GGUATCCCCCCCCCCCCCCC-3′ (SEQ ID NO: 54).

In other embodiments, the one or more candidate oligonucleotides or the modified oligonucleotide inhibit cGAS and TLR9 activity. Examples of the motif of such embodiments include 5′-CUUGUGAAAAGATTAUCUUC-3′ (SEQ ID NO: 27), 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In some embodiments, the one or more candidate oligonucleotides or the modified oligonucleotide inhibit cGAS and TLR9 activity, but do not substantially inhibit TLR7 activity. Examples of the motif of such embodiments include 5′-CUUGUGAAAAGATTAUCUUC-3′ (SEQ ID NO: 27).

In further embodiments, the one or more candidate oligonucleotides or the modified oligonucleotide inhibit cGAS, TLR7 and TLR9 activity. Examples of the motif of such embodiments include 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In alternative embodiments, the one or more candidate oligonucleotides or the modified oligonucleotide inhibit cGAS, but do not substantially inhibit TLR7 and/or TLR9.

In alternative embodiments, the one or more candidate oligonucleotides or modified oligonucleotides that inhibit cGAS comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of cGAS.

In alternative embodiments, the one or more candidate oligonucleotides or modified oligonucleotides that inhibit cGAS comprise or consist of any sequence in Tables 1, 1A, 2, 2A, 3, 3A, 4, 4A, 5, 5A or 6, with or without the particular modifications defined, that is shown in the Examples to inhibit the activity of cGAS and the oligonucleotide inhibits, or does not substantially inhibit, TLR3, TLR7, TLR8 and/or TLR9 activity.

With respect to the above two aspects, the method may include the further step of testing the ability of the one or more candidate oligonucleotides or the modified oligonucleotide to potentiate TLR8 activity, and optionally selecting an oligonucleotide which potentiates or does not substantially potentiate TLR8 activity. Accordingly, in some embodiments, the one or more candidate oligonucleotides or the modified oligonucleotide inhibit cGAS activity and potentiate TLR8 activity. In other embodiments, the one or more candidate oligonucleotides or the modified oligonucleotide inhibit cGAS activity and do not substantially potentiate TLR8 activity.

In any embodiment, the candidate oligonucleotide or modified oligonucleotide that inhibits cGAS activity is at least 15, 16, 17, 18, 19 or 20 nucleotides in length. In any embodiment, the candidate oligonucleotide or modified oligonucleotide that inhibits cGAS activity is at least 15 but less than or equal to 20 nucleotides in length.

In yet another aspect, the invention relates to a method for selecting or designing an oligonucleotide which inhibits TLR9 activity, the method comprising
i) scanning a polynucleotide, or complement thereof, for a region having a motif including a sequence selected from the group consisting of:
5′-G[G/C]CCT[C/G]-3′ (SEQ ID NO: 152);
5′-CUU-3′ (SEQ ID NO: 153), wherein the motif is within 10 bases of the 5′ and/or 3′ end of the oligonucleotide;

(SEQ ID NO: 27)
5′-CUUGUGAAAAGATTAUCUUC-3′;
(SEQ ID NO: 154)
5′-CUUCUCTCTGGTCCCAUCCC-3′;
(SEQ ID NO: 142)
5′-CCUUCTCTCTGGTCCCAUCC-3′;
(SEQ ID NO: 155)
5′-CCCUUCTCTCTGGTCCCAUC-3′;
(SEQ ID NO: 156)
5′-ACCCUTCTCTCTGGTCCCAU-3′;
(SEQ ID NO: 157)
5′-CUUCCACAATCAAGACAUUC-3′;
(SEQ ID NO: 158)
5′-CUUCGTGGGGTCCTTUUCAC-3′;
(SEQ ID NO: 159)
5′-CACUUCGTGGGGTCCUUUUC-3′;
(SEQ ID NO: 160)
5′-CCAACACTTCGTGGGGUCCU-3′;
(SEQ ID NO: 10)
5′-UCCGGCCTCGGAAGCUCUCU-3′;
(SEQ ID NO: 29)
5′-CCAUGTCCCAGGCCTCCAGU-3′;
(SEQ ID NO: 161)
5′-UUGGCCTGTGGATGCUUUGU-3′;
(SEQ ID NO: 162)
5′-AAAUGTCCTGGCCCTCACUG-3′;
(SEQ ID NO: 52)
5′-UCCGGCCTCGGAGTCUCCAU-3′;
(SEQ ID NO: 163)
5′-UCCGGCCTCGGCAGAUAUCG-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 51)
5′-UCCGGGTCGTAGTTGCUUCC-3′;
(SEQ ID NO: 164)
5′-CCUAGAAAGAAGCAAAGAUU-3′;
(SEQ ID NO: 165)
5′-GAUUAAAACAGATTAAUACA-3′;
(SEQ ID NO: 55)
5′-GGAUUAAAACAGATTAAUAC-3′;
(SEQ ID NO: 166)
5′-AAUUUAAAGCATGAAUAUUA-3′;
(SEQ ID NO: 41)
5′-GCGUAGTTTCTCTTCCUCCC-3′;
(SEQ ID NO: 167)
5′-UGACAAAACAATAATAACAG-3′;
(SEQ ID NO: 168)
5′-ACA-3′, wherein the motif is at or towards
the 5′ end of the oligonucleotide;
(SEQ ID NO: 169)
5′-CAC-3′, wherein the motif is at or towards
the 5′ end of the oligonucleotide;
(SEQ ID NO: 170)
5′-ACACTTCGTGGGGTCCTTTT-3′;
(SEQ ID NO: 86)
5′-GCGTGTCTGGAAGCTTCCTT-3′;
(SEQ ID NO: 171)
5′-TCAAAGGACTGAGGAAAGGG-3′;
(SEQ ID NO: 172)
5′-ATCCAACACTTCGTGGGGTC-3′;
(SEQ ID NO: 173)
5′-GCCCATCCATGAGGTCCTGG-3′;
(SEQ ID NO: 174)
5′-GGGTATCGAAAGAGTCTGGA-3′;
(SEQ ID NO: 175)
5′-GGTTTTGGCTGGGATCAAGT-3′;
(SEQ ID NO: 176)
5′-GCGACTATACGCGCAATATG-3′;
(SEQ ID NO: 85)
5′-ACTGACTGTCTTGAGGGTTC-3′;
(SEQ ID NO: 177)
5′-AACACTTCGTGGGGTCCTTT-3′;
(SEQ ID NO: 178)
5′-GTCCAAGATCAGCAGTCTCA-3′;
(SEQ ID NO: 91)
5′-ACAGTGTTGAGATACTCGGG-3′;
(SEQ ID NO: 179)
5′-TGGGCTGGAATCCGAGTTAT-3′;
(SEQ ID NO: 180)
5′-CGGCATCCACCACGTCGTCC-3′;
(SEQ ID NO: 181)
5′-GCGTATTATAGCCGATTAAC-3′;
(SEQ ID NO: 182)
5′-GGAGGTCTTGGCTTCGTGGA-3′;
(SEQ ID NO: 183)
5′-TGGGTTACGGCTCAGTATGG-3′;
(SEQ ID NO: 184)
5′-CCGCCATGTTTCTTCTTGGA-3′;
(SEQ ID NO: 185)
5′-AGCTTCGAGGCCCCAG-3′;
(SEQ ID NO: 186)
5′-GCCATGTTTCTTCTTG-3′;
(SEQ ID NO: 187)
5′-CACTTCGTGGGGTCCT-3′;
(SEQ ID NO: 188)
5′-CGGCCTCGGAAGCTCT-3′;
(SEQ ID NO: 110)
5′-ACACTTCGTGGGGTCC-3′;
(SEQ ID NO: 189)
5′-TGCACACTTCGTACCC-3′;
(SEQ ID NO: 190)
5′-CCACATCCTGTGGCTC-3′;
(SEQ ID NO: 191)
5′-CTGCAGCTTCCTTGTC-3′;
(SEQ ID NO: 192)
5′-ACTTCGTGGGGTCCTT-3′;
(SEQ ID NO: 193)
5′-CCCACTTGGCAGACCA-3′;
(SEQ ID NO: 194)
5′-GTCCCCTGTTGACTGG-3′;
(SEQ ID NO: 195)
5′-ACGTTCAGTCCTGTCC-3′;
(SEQ ID NO: 196)
5′-GGTCATTACAATAGCT-3′;
(SEQ ID NO: 197)
5′-TCGTGGGGTCCTTTTC-3′;
(SEQ ID NO: 106)
5′-GTGTCTGGAAGCTTCC-3′;
(SEQ ID NO: 104)
5′-ATGGCCTCCCATCTCC-3′;
(SEQ ID NO: 198)
5′-TGCTCCTCGGTCTCCC-3′;
(SEQ ID NO: 199)
5′-GCATCCACCACGTCGT-3′;
(SEQ ID NO: 200)
5′-CTTCGTGGGGTCCTTT-3′;
(SEQ ID NO: 201)
5′-AGGCCCTTCGCACTTC-3′;
5′-GCGGUATCCATGTCCCAGGC-3′;
5′-GCUGUTTCCATGTCCCAGGC-3′;
5′-GCUGUGTCCATGTCCCAGGC-3′
5′-GCCGUTTCCATGTCCCAGGC-3′;
5′-GCGGUATCC-3′;
5′-GCUGUTTCC-3′;
5′-GCUGUGTCC-3′;
5′-GCCGUTTCC-3′;
5′-CUUCGTGGGGTCCTTUUCAC-3′;
5′-CUUCGTGGG-3′;
5′-UCG-3′;
5′-ACG-3′;
5′-ACC-3′;
5′-CGC-3′;
5′-GAU-3′;
5′-GGG-3′;
5′-AGC-3′;
5′-UUC-3′;
5′-UUG-3′;
5′-CAC-3′;

and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR9 activity, and
iv) selecting an oligonucleotide which inhibits TLR9 activity.

In a related aspect, the invention resides in a method for increasing the TLR9 inhibitory activity of an oligonucleotide, the method comprising modifying the oligonucleotide such that the modified oligonucleotide comprises a motif including a sequence selected from the group consisting of:

(SEQ ID NO: 152)
5′-G[G/C]CCT[C/G]-3′;
(SEQ ID NO: 153)
5′-CUU-3′, wherein the motif is within 10 bases
of the 5′ and/or 3′ end of the oligonucleotide;
(SEQ ID NO: 27)
5′-CUUGUGAAAAGATTAUCUUC-3′;
(SEQ ID NO: 154)
5′-CUUCUCTCTGGTCCCAUCCC-3′;
(SEQ ID NO: 142)
5′-CCUUCTCTCTGGTCCCAUCC-3′;
(SEQ ID NO: 155)
5′-CCCUUCTCTCTGGTCCCAUC-3′;
(SEQ ID NO: 156)
5′-ACCCUTCTCTCTGGTCCCAU-3′;
(SEQ ID NO: 157)
5′-CUUCCACAATCAAGACAUUC-3′;
(SEQ ID NO: 158)
5′-CUUCGTGGGGTCCTTUUCAC-3′;
(SEQ ID NO: 159)
5′-CACUUCGTGGGGTCCUUUUC-3′;
(SEQ ID NO: 160)
5′-CCAACACTTCGTGGGGUCCU-3′;
(SEQ ID NO: 10)
5′-UCCGGCCTCGGAAGCUCUCU-3′;
(SEQ ID NO: 29)
5′-CCAUGTCCCAGGCCTCCAGU-3′;
(SEQ ID NO: 161)
5′-UUGGCCTGTGGATGCUUUGU-3′;
(SEQ ID NO: 162)
5′-AAAUGTCCTGGCCCTCACUG-3′;
(SEQ ID NO: 52)
5′-UCCGGCCTCGGAGTCUCCAU-3′;
(SEQ ID NO: 163)
5′-UCCGGCCTCGGCAGAUAUCG-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 51)
5′-UCCGGGTCGTAGTTGCUUCC-3′;
(SEQ ID NO: 164)
5′-CCUAGAAAGAAGCAAAGAUU-3′;
(SEQ ID NO: 165)
5′-GAUUAAAACAGATTAAUACA-3′;
(SEQ ID NO: 55)
5′-GGAUUAAAACAGATTAAUAC-3′;
(SEQ ID NO: 166)
5′-AAUUUAAAGCATGAAUAUUA-3′;
(SEQ ID NO: 41)
5′-GCGUAGTTTCTCTTCCUCCC-3′;
(SEQ ID NO: 167)
5′-UGACAAAACAATAATAACAG-3′;
(SEQ ID NO: 168)
5′-ACA-3′, wherein the motif is at or towards
the 5′ end of the oligonucleotide;
(SEQ ID NO: 169)
5′-CAC-3′, wherein the motif is at or towards
the 5′ end of the oligonucleotide;
(SEQ ID NO: 170)
5′-ACACTTCGTGGGGTCCTTTT-3′;
(SEQ ID NO: 86)
5′-GCGTGTCTGGAAGCTTCCTT-3′;
(SEQ ID NO: 171)
5′-TCAAAGGACTGAGGAAAGGG-3′;
(SEQ ID NO: 172)
5′-ATCCAACACTTCGTGGGGTC-3′;
(SEQ ID NO: 173)
5′-GCCCATCCATGAGGTCCTGG-3′;
(SEQ ID NO: 174)
5′-GGGTATCGAAAGAGTCTGGA-3′;
(SEQ ID NO: 175)
5′-GGTTTTGGCTGGGATCAAGT-3′;
(SEQ ID NO: 176)
5′-GCGACTATACGCGCAATATG-3′;
(SEQ ID NO: 85)
5′-ACTGACTGTCTTGAGGGTTC-3′;
(SEQ ID NO: 177)
5′-AACACTTCGTGGGGTCCTTT-3′;
(SEQ ID NO: 178)
5′-GTCCAAGATCAGCAGTCTCA-3′;
(SEQ ID NO: 91)
5′-ACAGTGTTGAGATACTCGGG-3′;
(SEQ ID NO: 179)
5′-TGGGCTGGAATCCGAGTTAT-3′;
(SEQ ID NO: 180)
5′-CGGCATCCACCACGTCGTCC-3′;
(SEQ ID NO: 181)
5′-GCGTATTATAGCCGATTAAC-3′;
(SEQ ID NO: 182)
5′-GGAGGTCTTGGCTTCGTGGA-3′;
(SEQ ID NO: 183)
5′-TGGGTTACGGCTCAGTATGG-3′;
(SEQ ID NO: 184)
5′-CCGCCATGTTTCTTCTTGGA-3′;
(SEQ ID NO: 185)
5′-AGCTTCGAGGCCCCAG-3′;
(SEQ ID NO: 186)
5′-GCCATGTTTCTTCTTG-3′;
(SEQ ID NO: 187)
5′-CACTTCGTGGGGTCCT-3′;
(SEQ ID NO: 188)
5′-CGGCCTCGGAAGCTCT-3′;
(SEQ ID NO: 110)
5′-ACACTTCGTGGGGTCC-3′;
(SEQ ID NO: 189)
5′-TGCACACTTCGTACCC-3′;
(SEQ ID NO: 190)
5′-CCACATCCTGTGGCTC-3′;
(SEQ ID NO: 191)
5′-CTGCAGCTTCCTTGTC-3′;
(SEQ ID NO: 192)
5′-ACTTCGTGGGGTCCTT-3′;
(SEQ ID NO: 193)
5′-CCCACTTGGCAGACCA-3′;
(SEQ ID NO: 194)
5′-GTCCCCTGTTGACTGG-3′;
(SEQ ID NO: 195)
5′-ACGTTCAGTCCTGTCC-3′;
(SEQ ID NO: 196)
5′-GGTCATTACAATAGCT-3′;
(SEQ ID NO: 197)
5′-TCGTGGGGTCCTTTTC-3′;
(SEQ ID NO: 106)
5′-GTGTCTGGAAGCTTCC-3′;
(SEQ ID NO: 104)
5′-ATGGCCTCCCATCTCC-3′;
(SEQ ID NO: 198)
5′-TGCTCCTCGGTCTCCC-3′;
(SEQ ID NO: 199)
5′-GCATCCACCACGTCGT-3′;
(SEQ ID NO: 200)
5′-CTTCGTGGGGTCCTTT-3′;
(SEQ ID NO: 201)
5′-AGGCCCTTCGCACTTC-3′;
5′-GCGGUATCCATGTCCCAGGC-3′;
5′-GCUGUTTCCATGTCCCAGGC-3′;
5′-GCUGUGTCCATGTCCCAGGC-3′
5′-GCCGUTTCCATGTCCCAGGC-3′;
5′-GCGGUATCC-3′;
5′-GCUGUTTCC-3′;
5′-GCUGUGTCC-3′;
5′-GCCGUTTCC-3′;
5′-CUUCGTGGGGTCCTTUUCAC-3′;
5′-CUUCGTGGG-3′;
5′-UCG-3′;
5′-ACG-3′;
5′-ACC-3′;
5′-CGC-3′;
5′-GAU-3′;
5′-GGG-3′;
5′-AGC-3′;
5′-UUC-3′;
5′-UUG-3′;
5′-CAC-3′;

and
a variant of the motifs or sequences thereof having at least about 75% sequence identity thereto;
wherein the U may be a T and/or the T may be a U.

In an embodiment, the present method further comprises testing the ability of the modified oligonucleotide to inhibit TLR9 activity, and selecting an oligonucleotide which inhibits TLR9 activity to a greater extent than the unmodified oligonucleotide.

In an embodiment, the step of modifying the oligonucleotide includes adding a sequence of nucleotides to the 5′ and/or 3′ end of the oligonucleotide such that the modified oligonucleotide comprises the motif.

In an embodiment, the step of modifying the oligonucleotide includes adding a sequence of nucleotides to the 5′ end of the oligonucleotide such that the modified oligonucleotide comprises the motif 5′-ACA-3′ (SEQ ID NO: 168), 5′-CAC-3′ (SEQ ID NO: 169), 5′-UCG-3′, 5′-ACG-3′, 5′-ACC-3′, 5′-CGC-3′, 5′-GAU-3′, 5′-GGG-3′, 5′-AGC-3′, 5′-UUC-3′, 5′-UUG-3′, or 5′-CAC-3′. In some embodiments, the step of modifying the oligonucleotide includes adding 5′-ACA-3′ (SEQ ID NO: 168), 5′-CAC-3′ (SEQ ID NO: 169), 5′-UCG-3′, 5′-ACG-3′, 5′-ACC-3′, 5′-CGC-3′, 5′-GAU-3′, 5′-GGG-3′, 5′-AGC-3′, 5′-UUC-3′, 5′-UUG-3′, or 5′-CAC-3′ or a portion or fragment thereof, to the 5′ end of the oligonucleotide.

In an embodiment, the step of modifying the oligonucleotide includes adding a sequence of nucleotides to the 5′ end of the oligonucleotide such that the modified oligonucleotide comprises the motif 5′-GCGGUATCC-3′, 5′-GCUGUTTCC-3′, 5′-GCUGUGTCC-3′, 5′-GCCGUTTCC-3′, or 5′-CUUCGTGGGGTCCTTUUCAC-3′, or 5′-CUUCGTGGG-3′. In some embodiments, the step of modifying the oligonucleotide includes adding 5′-GCGGUATCC-3′, 5′-GCUGUTTCC-3′, 5′-GCUGUGTCC-3′, 5′-GCCGUTTCC-3′, 5′-CUUCGTGGGGTCCTTUUCAC-3′, 5′-CUUCGTGGG-3′; or a portion or fragment thereof, to the 5′ end of the oligonucleotide.

In an embodiment of the two aforementioned aspects, the oligonucleotide does not bind or is not designed to bind a transcript that encodes TLR9 or a complement thereof.

In an embodiment of the two aforementioned aspects, the oligonucleotide binds or is designed to bind a target transcript that does not encode TLR9 or a complement thereof.

In an alternative embodiment of the two aforementioned aspects, the oligonucleotide binds or is designed to bind a target transcript that encodes TLR9 or a complement thereof.

For the two aforementioned aspects, the motif is suitably within 10 bases of the 5′ and/or 3′ end of the oligonucleotide. More particularly, the motif is suitably within 5 bases of the 5′ and/or 3′ end of the oligonucleotide. Even more particularly, the motif is suitably at or towards the 5′ and/or 3′ end of the oligonucleotide. Yet even more particularly, the motif is suitably at or towards the 5′ end of the oligonucleotide.

Examples of the motif of the above two aspects include, but are not limited to, those having the sequence 5′-CUU-3′ (SEQ ID NO: 153), 5′-CUT-3′ (SEQ ID NO: 202), 5′-CTT-3′ (SEQ ID NO: 203), 5′-UCG-3′, 5′-ACG-3′, 5′-ACC-3′, 5′-CGC-3′, 5′-GAU-3′, 5′-GGG-3′, 5′-AGC-3′, 5′-UUC-3′, 5′-UUG-3′, or 5′-CAC-3′.

Further examples of the motif of the above two aspects include, but are not limited to, those having the sequence 5′-GGCCTC-3′ (SEQ ID NO: 204), 5′-GGCCTG-3′ (SEQ ID NO: 205), or 5′-GCCCTC-3′ (SEQ ID NO: 206), wherein the T may be a U.

Additional examples of the motif of the above two aspects include, but are not limited to, those having the sequence 5′-ACA-3′ (SEQ ID NO: 168) or 5′-CAC-3′ (SEQ ID NO: 169).

In one particular embodiment, the motif of the above aspects has the sequence of 5′-GGCCTC-3′ (SEQ ID NO: 204), 5′-GGCCUC-3′ (SEQ ID NO: 207), 5′-UCCGGCCTCGGAAGCUCUCU-3′ (SEQ ID NO: 10) or a variant thereof having at least about 75% sequence identity thereto, wherein the U may be a T and/or the T may be a U.

In an embodiment of the two aforementioned aspects, one or more of the bases of the motif are a modified base and/or have a modified backbone.

In an embodiment of the two aforementioned aspects, the motif has the sequence 5′-mCmUmU-3′, 5′-mCmUT-3′, 5′-mUmCmG-3′, 5′-mAmCmG-3′, 5′-mAmCmC-3′, 5′-mCmGmC-3′, 5′-mGmAmU-3′, 5′-mGmGmG-3′, 5′-mAmGmC-3′, 5′-mUmUmC-3′, 5′-mUmUmG-3′ or 5′-mCmAmC-3′; wherein m is a modified base and/or has a modified backbone. For such embodiments, m is suitably a 2′-OMe modified base.

In an embodiment of the two aforementioned aspects, the motif has the sequence 5′-mGmGCCTC-3′, 5′-GGCCTmC-3′, 5′-mGmGmCCTG-3′ or 5′-GCCCTmC-3′, wherein the T may be a U and wherein m is a modified base and/or has a modified backbone. For such embodiments, m is suitably a 2′-OMe modified base.

In an embodiment of the two above aspects, the motif has the sequence 5′-mAmCmA-3′ or 5′-mCmAmC-3′, wherein m is a modified base and/or has a modified backbone. For such examples, m is suitably a 2′-LNA, a 2′-MOE and/or a 2′-OMe modified base.

In one particular embodiment, the motif of the above aspects has the sequence of 5′-mGmGCCTC-3′, 5′-mGmGCCUC-3′, 5′-mUmCmCmGmGCCTCGGAAGCmUmCmUmCmU-3′ or a variant thereof having at least about 75% sequence identity thereto, wherein the U may be a T and/or the T may be a U and wherein m is a modified base and/or has a modified backbone.

In one embodiment of the above two aspects, the motif has the sequence:

(SEQ ID NO: 200)
5′-CTTCGTGGGGTCCTTT-3′;
(SEQ ID NO: 187)
5′-CACTTCGTGGGGTCCT-3′;
(SEQ ID NO: 110)
5′-ACACTTCGTGGGGTCC-3′;
(SEQ ID NO: 189)
5′-TGCACACTTCGTACCC-3′;
(SEQ ID NO: 188)
5′-CGGCCTCGGAAGCTCT-3′;
(SEQ ID NO: 185)
5′-AGCTTCGAGGCCCCAG-3′;
(SEQ ID NO: 186)
5′-GCCATGTTTCTTCTTG-3′;
or
(SEQ ID NO: 191)
5′-CTGCAGCTTCCTTGTC-3′;

wherein the U may be a T and/or the T may be a U.

In particular embodiments of the above two aspects, the motif has the sequence:

5′-mCmTmTCGTGGGGTCCmTmTmT-3′;
5′-mCmAmCTTCGTGGGGTmCmCmT-3′;
5′-mAmCmACTTCGTGGGGmTmCmC-3′;
5′-mTmGmCACACTTCGTAmCmCmC-3′;
5′-mCmGmGCCTCGGAAGCmTmCmT-3′;
5′-mAmGmCTTCGAGGCCCmCmAmG-3′;
5′-mGmCmCATGTTTCTTCmTmTmG-3′;
or
5′-mCmTmGCAGCTTCCTTmGmTmC-3′;

wherein the U may be a T and/or the T may be a U and wherein m is a modified base and/or has a modified backbone. For such examples, m is suitably a 2′-LNA modified base.

In another embodiment of the above two aspects the motif has the sequence:

(SEQ ID NO: 170)
5′-ACACTTCGTGGGGTCCTTTT-3′;
(SEQ ID NO: 177)
5′-AACACTTCGTGGGGTCCTTT-3′;
(SEQ ID NO: 208)
5′-CAACACTTCGTGGGGTCCTT-3′;
(SEQ ID NO: 209)
5′-CCAACACTTCGTGGGGTCCT-3′;
(SEQ ID NO: 86)
5′-GCGTGTCTGGAAGCTTCCTT-3′;
(SEQ ID NO: 173)
5′-GCCCATCCATGAGGTCCTGG-3′;
(SEQ ID NO: 174)
5′-GGGTATCGAAAGAGTCTGGA-3′;
(SEQ ID NO: 179)
5′-TGGGCTGGAATCCGAGTTAT-3′;
(SEQ ID NO: 175)
5′-GGTTTTGGCTGGGATCAAGT-3′;
(SEQ ID NO: 171)
5′-TCAAAGGACTGAGGAAAGGG-3′;
(SEQ ID NO: 138)
5′-TCCGGCCTCGGAAGCTCTCT-3′;
(SEQ ID NO: 210)
5′-GGTGGTCCACAACCCCTTTC-3′;
(SEQ ID NO: 85)
5′-ACTGACTGTCTTGAGGGTTC-3′;
(SEQ ID NO: 181)
5′-GCGTATTATAGCCGATTAAC-3′;
or
(SEQ ID NO: 176)
5′-GCGACTATACGCGCAATATG-3′;

wherein the U may be a T and/or the T may be a U.

In particular embodiments of the above two aspects, the motif has the sequence:

5′-mAmCmAmCmTTCGTGGGGTCmCmTmTmTmT-3′;
5′-mAmAmCmAmCTTCGTGGGGTmCmCmTmTmT-3′;
5′-mCmAmAmCmACTTCGTGGGGmTmCmCmTmT-3′;
5′-mCmCmAmAmCACTTCGTGGGmGmTmCmCmT-3′;
5′-mGmCmGmTmGTCTGGAAGCTmTmCmCmTmT-3′;
5′-mGmCmCmCmATCCATGAGGTmCmCmTmGmG-3′;
5′-mGmGmGmTmATCGAAAGAGTmCmTmGmGmA-3′;
5′-mTmGmGmGmCTGGAATCCGAmGmTmTmAmT-3′;
5′-mGmGmTmTmTTGGCTGGGATmCmAmAmGmT-3′;
5′-mTmCmAmAmAGGACTGAGGAmAmAmGmGmG-3′;
5′-mTmCmCmGmGCCTCGGAAGCmTmCmTmCmT-3′;
5′-mGmGmTmGmGTCCACAACCCmCmTmTmTmC-3′;
5′-mAmCmTmGmACTGTCTTGAGmGmGmTmTmC-3′;
5′-mGmCmGmTmATTATAGCCGAmTmTmAmAmC-3′;
or
5′-mGmCmGmAmCTATACGCGCAmAmTmAmTmG-3′;

wherein the U may be a T and/or the T may be a U and wherein m is a modified base and/or has a modified backbone. For such examples, m is suitably a 2′-MOE modified base.

In particular embodiments of the above two aspects, the motif has the sequence:

5′-mUmCmCmGmGCCTCGGAAGCmUmCmUmCmU-3′;
5′-mUmCmCmGmGCCTCGGAGTCmUmCmCmAmU-3′;
5′-mUmCmCmGmGCCTCGGCAGAmUmAmUmCmG-3′;
5′mGmCmGmGmUATCCATGTCCmCmAmGmGmC-3′;
5′-mGmCmUmGmUTTCCATGTCCmCmAmGmGmC-3′;
5′-mGmCmUmGmUGTCCATGTCCmCmAmGmGmC-3′;
5′-mGmCmCmGmUTTCCATGTCCmCmAmGmGmC-3′;
5′-mGmCmGmGmUATCC-3′;
5′-mGmCmUmGmUTTCC-3′;
5′-mGmCmUmGmUGTCC-3′;
5′-mGmCmCmGmUTTCC-3′;
5′-mCmUmUmCmGTGGGGTCCTTmUmUmCmAmC;
and
5′-mCmUmUmCmGTGGG-3′;

wherein the T may be a U and wherein m is a modified base and/or has a modified backbone. Preferably, each base has a modified backbone, and the modified backbone is phosphorothioate.

In particular embodiments of the above two aspects, the motif has the sequence:

5′-mG*mC*mG*mG*mU*A*T*C*C*A*T*G*T*C*C*mC*mA*mG*
mG*mC-3′;
5′-mU*mC*mC*mG*mG*C*C*T*C*G*G*A*A*G*C*mU*mC*mU*
mC*mU-3′;
5′-mU*mC*mC*mG*mG*C*C*T*C*G*G*A*G*T*C*mU*mC*mC*
mA*mU-3′;
5′-mU*mC*mC*mG*mG*C*C*T*C*G*G*C*A*G*A*mU*mA*mU*
mC*mG-3′;
5′-mG*mC*mU*mG*mU*T*T*C*C*A*T*G*T*C*C*mC*mA*mG
*mG*mC-3′;
5′-mG*mC*mU*mG*mU*G*T*C*C*A*T*G*T*C*C*mC*mA*mG*
mG*mC-3′;
5′-mG*mC*mC*mG*mU*T*T*C*C*A*T*G*T*C*C*mC*mA*mG*
mG*mC-3′;
5′-mG*mC*mG*mG*mU*A*T*C*C*-3′;
5′-mG*mC*mU*mG*mU*T*T*C*C*-3′;
5′-mG*mC*mU*mG*mU*G*T*C*C*-3′;
5′-mG*mC*mC*mG*mU*T*T*C*C*-3′;
5′-mC*mU*mU*mC*mG*T*G*G*G*G*T*C*C*T*T*mU*mU*mC*
mA*mC-3′;
and
5′-mC*mU*mU* mC*mG*T*G*G*G*-3′;

wherein the U may be a T and/or the T may be a U and wherein ‘m’ indicates 2′OMe base, and * denotes the phosphorothioate backbone.

In still another aspect, the invention provides a method for selecting or designing an oligonucleotide which inhibits TLR9 activity, the method comprising
i) scanning a polynucleotide, or complement thereof, for regions having at least about 50% adenine bases;
ii) producing one or more candidate oligonucleotides comprising a 5′ region, a 3′ region and a middle region comprising ribonucleic acid, deoxyribonucleic acid, or combination thereof, bases, which optionally have a modified backbone, wherein one or both of the 5′ region and the 3′ region comprise bases which are modified and/or which have a modified backbone, and wherein at least about 50% of the bases of the middle region are adenine bases;
iii) testing the ability of the one or more candidate oligonucleotides to inhibit TLR9 activity, and
iv) selecting an oligonucleotide which inhibits TLR9 activity.

In an embodiment, the oligonucleotide comprises a motif having a sequence of 5′-[T/G][A/T][G/A/T]AA[A/C][A/G][G/C/A]A[T/G][T/G/C]A[A/T]-3′ (SEQ ID NO: 211), wherein the T may be a U.

Suitably, the 5′ region and/or the 3′ region are about 5 bases in length and the middle region is about 10 bases in length, wherein the middle region comprises at least five adenine bases. In an embodiment, two, three and/or four of the at least five adenine bases are in a continuous sequence.

In an embodiment, the oligonucleotide does not bind or is not designed to bind a transcript that encodes TLR9 or a complement thereof In an embodiment, the oligonucleotide binds or is designed to bind a target transcript that does not encode TLR9 or a complement thereof.

In any embodiment of the above aspects, the motif comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR9.

With respect to the above three aspects, the method may include the further step of testing the ability of the one or more candidate oligonucleotides or the modified oligonucleotide to inhibit TLR3, TLR7 and/or cGAS activity, and optionally selecting an oligonucleotide which inhibits or does not substantially inhibit TLR3, TLR7 and/or cGAS activity.

In particular embodiments, the one or more candidate oligonucleotides or the modified oligonucleotide inhibit TLR9 and TLR7 activity. Examples of the motif of such embodiments include 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52), 5′-GCGGUATCCATAGTCUCCAU-3′ (SEQ ID NO: 48), 5′-GAUUAAAACAGATTAAUACA-3′ (SEQ ID NO: 165), 5′-UGACAAAACAATAATAACAG-3′ (SEQ ID NO: 167) and 5′-CCAACACTTCGTGGGGUCCU-3′ (SEQ ID NO: 160).

In particular embodiments, the one or more candidate oligonucleotides or the modified oligonucleotide inhibit TLR9 and TLR7 activity, but do not substantially inhibit cGAS activity. Examples of the motif of such embodiments include 5′-GAUUAAAACAGATTAAUACA-3′ (SEQ ID NO: 165) and 5′-UGACAAAACAATAATAACAG-3′ (SEQ ID NO: 167).

In other embodiments, the one or more candidate oligonucleotides or the modified oligonucleotide inhibit TLR9 and cGAS activity. Examples of the motif of such embodiments include 5′-CUUGUGAAAAGATTAUCUUC-3′ (SEQ ID NO: 27), 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In other embodiments, the one or more candidate oligonucleotides or the modified oligonucleotide inhibit TLR9 and cGAS activity, but do not substantially inhibit TLR7 activity. Examples of the motif of such embodiments include 5′-CUUGUGAAAAGATTAUCUUC-3′ (SEQ ID NO: 27).

In further embodiments, the one or more candidate oligonucleotides or the modified oligonucleotide inhibit TLR9, TLR7 and cGAS activity. Examples of the motif of such embodiments include 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In alternative embodiments, the one or more candidate oligonucleotides or the modified oligonucleotide inhibit TLR9, but do not substantially inhibit TLR7 and/or cGAS. Examples of the motif of such embodiments include 5′-CACUUCGTGGGGTCCUUUUC-3′ (SEQ ID NO: 159).

In alternative embodiments, the one or more candidate oligonucleotides or modified oligonucleotides that inhibit TLR9 comprise or consist of any sequence in Tables 1, 1A, 2, 2A, 3, 3A, 4, 4A, 5, 5A or 6, with or without the particular modifications defined, that is shown in the Examples to inhibit the activity of TLR9.

In alternative embodiments, the one or more candidate oligonucleotides or modified oligonucleotides that inhibit TLR9 comprise or consist of any sequence in Tables 1, 1A, 2, 2A, 3, 3A, 4, 4A, 5, 5A or 6, with or without the particular modifications defined, that is shown in the Examples to inhibit the activity of TLR9 and the oligonucleotide inhibits, or does not substantially inhibit, TLR3, TLR8, TLR7 and/or cGAS activity.

With respect to the above two aspects, the method may include the further step of testing the ability of the one or more candidate oligonucleotides or the modified oligonucleotide to potentiate TLR8 activity, and optionally selecting an oligonucleotide which potentiates or does not substantially potentiate TLR8 activity. Accordingly, in some embodiments, the one or more candidate oligonucleotides or the modified oligonucleotide inhibit TLR9 activity and potentiate TLR8 activity. In other embodiments, the one or more candidate oligonucleotides or the modified oligonucleotide inhibit TLR9 activity and do not substantially potentiate TLR8 activity.

In any embodiment, the candidate oligonucleotide or modified oligonucleotide that inhibits TLR9 activity is at least 3, 4, 5, 6, 7, 8, 9, 10, 11, 12, 13, 14, 15, 16, 17, 18, 19, 20, 30, 40 or 50 nucleotides in length. In one embodiment, the candidate oligonucleotide or modified oligonucleotide that inhibits TLR9 activity is at least 3 but less than or equal to 20 nucleotides in length, optionally at least 9 but less than or equal to 20 nucleotides in length.

In yet another aspect, the invention provides a method for selecting or designing an oligonucleotide which inhibits TLR7 activity, the method comprising:
i) scanning a polynucleotide, or complement thereof, for a region having a motif including a sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 212)
5′-A[G/A][U/G]C[U/C]C[U/C][C/A]U-3′;
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 8)
5′-CGUGUC-3′;
(SEQ ID NO: 30)
5′-GCAGUCTCCATGTCCCAGGC-3′;
(SEQ ID NO: 38)
5′-GAUGGTTCCAGTCCCUCUUC-3′;
(SEQ ID NO: 31)
5′-AGCAGTCTCCATGTCCCAGG-3′;
(SEQ ID NO: 36)
5′-GGGUCTCCTCCACACCCUUC-3′;
(SEQ ID NO: 28)
5′-GGUGGCCACAGGCAACGUCA-3′;
(SEQ ID NO: 46)
5′-GCCGUTTCCATGTCCCAGGC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 47)
5′-GCCGUGTCCATGTCCCAGGC-3′;
(SEQ ID NO: 151)
5′-GCGGUAUCCAUGUCCCAGGC-3′;
(SEQ ID NO: 54)
5′-GGUATCCCCCCCCCCCCCCC-3′;
(SEQ ID NO: 145)
5′-GUCCCATCCCTTCTGCUGCC-3′;
(SEQ ID NO: 213)
5′-UUCUCTCTGGTCCCAUCCCU-3′;
(SEQ ID NO: 214)
5′-GUUCAGTCAGATCGCUGGGA-3′;
(SEQ ID NO: 215)
5′-AUGACATTTCGTGGCUCCUA-3′;
(SEQ ID NO: 216)
5′-UCUCCATGTCCCAGGCCUCC-3′;
(SEQ ID NO: 217)
5′-AGUCUCCATGTCCCAGGCCU-3′;
(SEQ ID NO: 29)
5′-CCAUGTCCCAGGCCTCCAGU-3′;
(SEQ ID NO: 37)
5′-GCAAGGCAGAGAAACUCCAG-3′;
(SEQ ID NO: 55)
5′-GGAUUAAAACAGATTAAUAC-3′;
(SEQ ID NO: 40)
5′-AGCCGAACAGAAGGAGCGUC-3′;
(SEQ ID NO: 10)
5′-UCCGGCCTCGGAAGCUCUCU-3′;
(SEQ ID NO: 52)
5′-UCCGGCCTCGGAGTCUCCAU-3′;
(SEQ ID NO: 48)
5′-GCGGUATCCATAGTCUCCAU-3′;
(SEQ ID NO: 165)
5′-GAUUAAAACAGATTAAUACA-3′;
(SEQ ID NO: 167)
5′-UGACAAAACAATAATAACAG-3′;
(SEQ ID NO: 160)
5′-CCAACACTTCGTGGGGUCCU-3′;
(SEQ ID NO: 21)
5′-GUGUCCTTCATGCTTUGGAU-3′;
(SEQ ID NO: 218)
5′-GUCCCAGGCCTCCAGUGUCU-3′;
(SEQ ID NO: 161)
5′-UUGGCCTGTGGATGCUUUGU-3′;
(SEQ ID NO: 219)
5′-GUCCGTACCTCCACCCACCG-3′;
(SEQ ID NO: 220)
5′-GUGUUTTTAATTTTGUAGAG-3′;
(SEQ ID NO: 221)
5′-GUCAAACCTAGAAAGAAGCA-3′;
(SEQ ID NO: 222)
5′-GGUCUCCTCCACACCCUUCU-3′;
(SEQ ID NO: 146)
5′-GUCUCCTCCACACCCUUCUC-3′;
(SEQ ID NO: 223)
5′-UGAUGATGCTTGCAGGAGGC-3′;
(SEQ ID NO: 20)
5′-UUAAATAATCTAGTTUGAAG-3′;
(SEQ ID NO: 224)
5′-AAAGCAGTCTCCATGUCCCA-3′;
(SEQ ID NO: 41)
5′-GCGUAGTTTCTCTTCCUCCC-3′;
(SEQ ID NO: 35)
5′-UCUGGTCCCATCCCTUCUGC-3′;
(SEQ ID NO: 225)
5′-UAUUUCCACATGCCCAGUGU-3′;
(SEQ ID NO: 39)
5′-UGUUUCCCCGGAGAGCAAUG-3′;
(SEQ ID NO: 226)
5′-UUAGCTCCTTGCCTCGUUCC-3′;
(SEQ ID NO: 33)
5′-AUUUCCACATGCCCAGUGUU-3′;
(SEQ ID NO: 227)
5′-UGGCGTAGTTTCTCTUCCUC-3′;
(SEQ ID NO: 228)
5′-UGACATTTCGTGGCTCCUAC-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 229)
5′-UUGUGAAAAGATTATCUUCU-3′;
(SEQ ID NO: 230)
5′-UUUGAAATTCAGAAGAUUUG-3′;
(SEQ ID NO: 231)
5′-AAGCAGTCTCCATGTCCCAG-3′;
(SEQ ID NO: 232)
5′-AGGAUTAAAACAGATUAAUA-3′;
(SEQ ID NO: 23)
5′-AGAUUATCTTCTTTTAAUUU-3′;
(SEQ ID NO: 148)
5′-UCCCAACTCTTCTAACUCGU-3′;
(SEQ ID NO: 233)
5′-UAAAATAAGGGGAATAGGGG-3′;
(SEQ ID NO: 32)
5′-AGUGGCACATACCACACCCU-3′;
(SEQ ID NO: 234)
5′-AAGAUTATCTTCTTTUAAUU-3′;
(SEQ ID NO: 22)
5′-AGAAAGAAGCAAAGAUUCAA-3′;
(SEQ ID NO: 235)
5′-UCCCATCCCTTCTGCUGCCA-3′;
(SEQ ID NO: 156)
5′-ACCCUTCTCTCTGGTCCCAU-3′;
(SEQ ID NO: 236)
5′-AAUAUCTGCTGCCCACCUUC-3′;
(SEQ ID NO: 237)
5′-UCUCUCTGGTCCCATCCCUU-3′;
(SEQ ID NO: 238)
5′-AGGCCTCCAGTGTCTUCUCC-3′;
(SEQ ID NO: 239)
5′-CAAGCCCCAGCGTTCCUCCG-3′;
(SEQ ID NO: 162)
5′-AAAUGTCCTGGCCCTCACUG-3′;
(SEQ ID NO: 166)
5′-AAUUUAAAGCATGAAUAUUA-3′;
(SEQ ID NO: 24)
5′-AAAAGATTATCTTCTUUUAA-3′;
(SEQ ID NO: 144)
5′-AUAUCTGCTGCCCACCUUCU-3′;
(SEQ ID NO: 240)
5′-CAGUCTCCATGTCCCAGGCC-3′;
(SEQ ID NO: 241)
5′-AAAGATTATCTTCTTUUAAU-3′;
(SEQ ID NO: 155)
5′-CCCUUCTCTCTGGTCCCAUC-3′;
(SEQ ID NO: 9)
5′-AUGGCCTTTCCGTGCCAAGG-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 242)
5′-GGGUATCGAAAGAGTCUGGA-3′;
(SEQ ID NO: 243)
5′-CUUGCACGTGGCTTCGUCUC-3′;
(SEQ ID NO: 244)
5′-GUGUCCTTGCACGTGGCUUC-3′;
(SEQ ID NO: 245)
5′-GUAAAAAGCTTTTGAAGUGA-3′;
(SEQ ID NO: 246)
5′-AUGCCATCCACTTGAUAGGC-3′;
(SEQ ID NO: 247)
5′-UGAAGTAAAAATCAAUAGCG-3′;
(SEQ ID NO: 248)
5′-AAGGCCCTTCGCACTUCUUA-3′;
(SEQ ID NO: 249)
5′-GUACUCGTCGGCATCCACCA-3′;
(SEQ ID NO: 250)
5′-GUCCUTGCACGTGGCUUCGU-3′;
(SEQ ID NO: 251)
5′-GCCCATCCATGAGGTCCUGG-3′;
(SEQ ID NO: 252)
5′-GUAAAAGGAGAAAACUAUCU-3′;
(SEQ ID NO: 253)
5′-UUGAAGTGAAGTAAAAGGAG-3′;
(SEQ ID NO: 254)
5′-GUUACTCGTGCCTTGGCAAA-3′;
(SEQ ID NO: 255)
5′-GUCCAAGATCAGCAGUCUCA-3′;
(SEQ ID NO: 256)
5′-UUCAATGGGAGAATAAAGCA-3′;
(SEQ ID NO: 257)
5′-GCAAGGCCCTTCGCACUUCU-3′;
(SEQ ID NO: 258)
5′-GGGUCCACCACTAGCCAGUA-3′;
(SEQ ID NO: 259)
5′-GUAGAGAAATTATTTUAGGA-3′;
(SEQ ID NO: 260)
5′-AUCCACCACGTCGTCCAUGU-3′;
(SEQ ID NO: 261)
5′-GGCAUCCACCACGTCGUCCA-3′;
(SEQ ID NO: 262)
5′-UUACUTTAAAAGCAAAAGGA-3′;
(SEQ ID NO: 263)
5′-UUUGAAGTGAAGTAAAAGGA-3′;
(SEQ ID NO: 264)
5′-GAAGUGAAGTAAAAGGAGAA-3′;
(SEQ ID NO: 265)
5′-GGCCATCTCTGCTTCUUGGU-3′;
(SEQ ID NO: 266)
5′-UGGGCTGGAATCCGAGUUAU-3′;
(SEQ ID NO: 267)
5′-GGAGATTTCAGAGCAGCUUC-3′;
(SEQ ID NO: 268)
5′-UUUACGGTTTTCAGAAUAUC-3′;
(SEQ ID NO: 269)
5′-GCGUGTCTGGAAGCTUCCUU-3′;
(SEQ ID NO: 270)
5′-GCUUATTTTAAGCATAUUAA-3′;
(SEQ ID NO: 271)
5′-UUAUUTTAAGCATATUAAAA-3′;
(SEQ ID NO: 272)
5′-UUCUGCAGCTTCCTTGUCCU-3′;
(SEQ ID NO: 273)
5′-AUUACTTTAAAAGCAAAAGG-3′;
(SEQ ID NO: 274)
5′-AUUUUAAGCATATTAAAAAG-3′;
(SEQ ID NO: 275)
5′-UGUGGCTTGTCCTCAGACAU-3′;
(SEQ ID NO: 276)
5′-AAAAGGAGAAAACTAUCUUC-3′;
(SEQ ID NO: 277)
5′-GGGUCCATACCCAAGGCAUC-3′;
(SEQ ID NO: 278)
5′-ACAGUGTTGAGATACUCGGG-3′;
(SEQ ID NO: 279)
5′-GGAUCTGCATGCCCTCAUCU-3′;
(SEQ ID NO: 280)
5′-AGUAAAAAGCTTTTGAAGUG-3′;
(SEQ ID NO: 281)
5′-GUCGUGGCAAATAGTCCUAG-3′;
(SEQ ID NO: 282)
5′-GGAGATCAGATGAGAGGAGC-3′;
(SEQ ID NO: 283)
5′-GUGGUTAAGTACATGAGCUC-3′;
(SEQ ID NO: 284)
5′-GGACACTTAGCTGTTCCUCG-3′;
(SEQ ID NO: 285)
5′-GUCUCTACTGTTACCUCUGA-3′;
(SEQ ID NO: 286)
5′-GAGUUCTTCGTAGGCUUCUG-3′;
(SEQ ID NO: 287)
5′-AAAGUCAAAAAGAAAAACUG-3′;
(SEQ ID NO: 288)
5′-AAAAGTGGGAAATAAAGGUU-3′;
(SEQ ID NO: 289)
5′-AGUUUATAGATTTCAAGUAG-3′;
(SEQ ID NO: 290)
5′-AAAAAGTGGGAAATAAAGGU-3′;
(SEQ ID NO: 291)
5′-UUUAUATTACAAAGCUACUU-3′;
(SEQ ID NO: 292)
5′-UGCUATTCATATTTTUAUUU-3′;
(SEQ ID NO: 56)
5′-GGUAU-3′;
(SEQ ID NO: 293)
5′-[G/A/C][G/A][G/A/C][T/A/C]TC-3′;
(SEQ ID NO: 294)
5′-[G/A/C]G[G/A/C][T/A/C]TC-3′;
(SEQ ID NO: 295)
5′-GGCTTC-3′;
(SEQ ID NO: 296)
5′-GGCATC-3′;
(SEQ ID NO: 297)
5′-AGCTTC-3′;
(SEQ ID NO: 298)
5′-GGAATC-3′;
(SEQ ID NO: 299)
5′-CACATC-3′;
(SEQ ID NO: 204)
5′-GGCCTC-3′;
(SEQ ID NO: 300)
5′-CACTTC-3′;
(SEQ ID NO: 301)
5′-AAGATC-3′;
(SEQ ID NO: 302)
5′-TGTCCTTGCACGTGGCTTCG-3′;
(SEQ ID NO: 94)
5′-TTTGCACACTTCGTACCCAA-3′;
(SEQ ID NO: 303)
5′-GTCCACATCCTGTGGCTCGT-3′;
(SEQ ID NO: 304)
5′-TGTGATGGCCTCCCATCTCC-3′;
(SEQ ID NO: 175)
5′-GGTTTTGGCTGGGATCAAGT-3′;
(SEQ ID NO: 93)
5′-GGTGTCCTTGCACGTGGCTT-3′;
(SEQ ID NO: 305)
5′-GGTCCATACCCAAGGCATCC-3′;
(SEQ ID NO: 306)
5′-GTGTCTTCATCGGCCCTGCC-3′;
(SEQ ID NO: 89)
5′-GTCTTGGCTTCGTGGAGCAG-3′;
(SEQ ID NO: 95)
5′-GCTGACAAAGATTCACTGGT-3′;
(SEQ ID NO: 103)
5′-GAAAGGTTATGCAAGG-3′;
(SEQ ID NO: 307)
5′-GACTATACGCGCAATA-3′;
(SEQ ID NO: 308)
5′-TGTGATGGCCTCCCAT-3′;
(SEQ ID NO: 114)
5′-TCCAACACTTCGTGGG-3′;
(SEQ ID NO: 106)
5′-GTGTCTGGAAGCTTCC-3′;
(SEQ ID NO: 98)
5′-CTTGAAGCATCGTATC-3′;
(SEQ ID NO: 309)
5′-TCGTAGTTGCTTCCTA-3′;
(SEQ ID NO: 105)
5′-CGCTTTTCTGTCTGGT-3′;
(SEQ ID NO: 310)
5′-GGCTGGAATCCGAGTT-3′;
(SEQ ID NO: 100)
5′-GATAGCACCTTCAGCA-3′;
(SEQ ID NO: 311)
5′-AGGACTCCAGATGTTT-3′;
(SEQ ID NO: 312)
5′-GTGATCTTGACATGCT-3′;
(SEQ ID NO: 313)
5′-AGATTTCAGAGCAGCT-3′;
(SEQ ID NO: 314)
5′-GGTTACGGCTCAGTAT-3′;
(SEQ ID NO: 315)
5′-GTTCAGTCCTGTCCAT-3′;
(SEQ ID NO: 316)
5′-AGGTCTTGGCTTCGTG-3′;
(SEQ ID NO: 191)
5′-CTGCAGCTTCCTTGTC-3′;
(SEQ ID NO: 317)
5′-GTCCTTGCACGTGGCT-3′;
(SEQ ID NO: 318)
5′-GTCTCTGGAGCTTCCT-3′;
(SEQ ID NO: 319)
5′-GGTCTTGGCTTCGTGG-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
5′-GUC-3′;
5′-GUG-3′;
5′-GUU-3′;
5′-GGC-3′;
5′-AUC-3′;
5′-GAG-3′;
5′-GGA-3′;
5′-TTT-3′;
5′-TCT-3′;
5′-GAA-3′;
5′-GAC-3′;
5′-GAU-3′;
5′-AUG-3′;
5′-GCG-3′;
5′-UUC-3′;
5′-GCC-3′;
5′-GGG-3′;
5′-AUU-3′;
5′-GCA-3′;
5′-AGC-3′;
5′-AAC-3′;
5′-CCA-3′;
5′-UGC-3′;
5′-CAA-3′;
5′-CGG-3′;
5′-ACC-3′;
5′-AGA-3′;
5′-TTT-3′;
5′-TCT-3′;
and

and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR7 activity, and
iv) selecting an oligonucleotide which inhibits TLR7 activity.

In one embodiment, step i) includes scanning a polynucleotide, or complement thereof, for the motif with the sequence of 5′-GGUAU-3′ (SEQ ID NO: 56), a portion or fragment thereof, or a variant having at least about 75% sequence identity thereto, wherein the U may be a T. Suitably, the motif of 5′-GGUAU-3′ (SEQ ID NO: 56) is at or towards a 5′ end of the oligonucleotide.

In some embodiments, step i) includes scanning a polynucleotide, or complement thereof, for the motif with the sequence of 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60), 5′-GUC-3′; 5′-GUG-3′; 5′-GUU-3′; 5′-GGC-3′; 5′-AUC-3′; 5′-GAA-3′; 5′-GAG-3′; 5′-GGA-3′; 5′-GAC-3′; 5′-GAU-3′; 5′-AUG-3′; 5′-GCG-3′; 5′-UUC-3′; 5′-GCC-3′; 5′-GGG-3′; 5′-AUU-3′; 5′-GCA-3′; 5′-AGC-3′; 5′-AAC-3′; 5′-CCA-3′; 5′-UGC-3′; 5′-CAA-3′; 5′-CGG-3′; 5′-ACC-3′; 5′-AGA-3′; 5′-TTT-3′; or 5′-TCT-3′ or a variant having at least about 75% sequence identity thereto, wherein the U may be a T. Suitably, the motif of such embodiments is at or towards a 5′ end of the oligonucleotide.

In still another aspect, the invention resides in a method for increasing the TLR7 inhibitory activity of an oligonucleotide, the method comprising modifying the oligonucleotide such that the modified oligonucleotide comprises a motif including a sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 212)
5′-A[G/A][U/G]C[U/C]C[U/C][C/A]U-3′;
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 8)
5′-CGUGUC-3′;
(SEQ ID NO: 30)
5′-GCAGUCTCCATGTCCCAGGC-3′;
(SEQ ID NO: 38)
5′-GAUGGTTCCAGTCCCUCUUC-3′;
(SEQ ID NO: 31)
5′-AGCAGTCTCCATGTCCCAGG-3′;
(SEQ ID NO: 36)
5′-GGGUCTCCTCCACACCCUUC-3′;
(SEQ ID NO: 28)
5′-GGUGGCCACAGGCAACGUCA-3′;
(SEQ ID NO: 46)
5′-GCCGUTTCCATGTCCCAGGC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 47)
5′-GCCGUGTCCATGTCCCAGGC-3′;
(SEQ ID NO: 151)
5′-GCGGUAUCCAUGUCCCAGGC-3′;
(SEQ ID NO: 54)
5′-GGUATCCCCCCCCCCCCCCC-3′;
(SEQ ID NO: 145)
5′-GUCCCATCCCTTCTGCUGCC-3′;
(SEQ ID NO: 213)
5′-UUCUCTCTGGTCCCAUCCCU-3′;
(SEQ ID NO: 214)
5′-GUUCAGTCAGATCGCUGGGA-3′;
(SEQ ID NO: 215)
5′-AUGACATTTCGTGGCUCCUA-3′;
(SEQ ID NO: 216)
5′-UCUCCATGTCCCAGGCCUCC-3′;
(SEQ ID NO: 217)
5′-AGUCUCCATGTCCCAGGCCU-3′;
(SEQ ID NO: 29)
5′-CCAUGTCCCAGGCCTCCAGU-3′;
(SEQ ID NO: 37)
5′-GCAAGGCAGAGAAACUCCAG-3′;
(SEQ ID NO: 55)
5′-GGAUUAAAACAGATTAAUAC-3′;
(SEQ ID NO: 40)
5′-AGCCGAACAGAAGGAGCGUC-3′;
(SEQ ID NO: 10)
5′-UCCGGCCTCGGAAGCUCUCU-3′;
(SEQ ID NO: 52)
5′-UCCGGCCTCGGAGTCUCCAU-3′;
(SEQ ID NO: 48)
5′-GCGGUATCCATAGTCUCCAU-3′;
(SEQ ID NO: 165)
5′-GAUUAAAACAGATTAAUACA-3′;
(SEQ ID NO: 167)
5′-UGACAAAACAATAATAACAG-3′;
(SEQ ID NO: 160)
5′-CCAACACTTCGTGGGGUCCU-3′;
(SEQ ID NO: 21)
5′-GUGUCCTTCATGCTTUGGAU-3′;
(SEQ ID NO: 218)
5′-GUCCCAGGCCTCCAGUGUCU-3′;
(SEQ ID NO: 161)
5′-UUGGCCTGTGGATGCUUUGU-3′;
(SEQ ID NO: 219)
5′-GUCCGTACCTCCACCCACCG-3′;
(SEQ ID NO: 220)
5′-GUGUUTTTAATTTTGUAGAG-3′;
(SEQ ID NO: 221)
5′-GUCAAACCTAGAAAGAAGCA-3′;
(SEQ ID NO: 222)
5′-GGUCUCCTCCACACCCUUCU-3′;
(SEQ ID NO: 146)
5′-GUCUCCTCCACACCCUUCUC-3′;
(SEQ ID NO: 223)
5′-UGAUGATGCTTGCAGGAGGC-3′;
(SEQ ID NO: 20)
5′-UUAAATAATCTAGTTUGAAG-3′;
(SEQ ID NO: 224)
5′-AAAGCAGTCTCCATGUCCCA-3′;
(SEQ ID NO: 41)
5′-GCGUAGTTTCTCTTCCUCCC-3′;
(SEQ ID NO: 35)
5′-UCUGGTCCCATCCCTUCUGC-3′;
(SEQ ID NO: 225)
5′-UAUUUCCACATGCCCAGUGU-3′;
(SEQ ID NO: 39)
5′-UGUUUCCCCGGAGAGCAAUG-3′;
(SEQ ID NO: 226)
5′-UUAGCTCCTTGCCTCGUUCC-3′;
(SEQ ID NO: 33)
5′-AUUUCCACATGCCCAGUGUU-3′;
(SEQ ID NO: 227)
5′-UGGCGTAGTTTCTCTUCCUC-3′;
(SEQ ID NO: 228)
5′-UGACATTTCGTGGCTCCUAC-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 229)
5′-UUGUGAAAAGATTATCUUCU-3′;
(SEQ ID NO: 230)
5′-UUUGAAATTCAGAAGAUUUG-3′;
(SEQ ID NO: 231)
5′-AAGCAGTCTCCATGTCCCAG-3′;
(SEQ ID NO: 232)
5′-AGGAUTAAAACAGATUAAUA-3′;
(SEQ ID NO: 23)
5′-AGAUUATCTTCTTTTAAUUU-3′;
(SEQ ID NO: 148)
5′-UCCCAACTCTTCTAACUCGU-3′;
(SEQ ID NO: 233)
5′-UAAAATAAGGGGAATAGGGG-3′;
(SEQ ID NO: 32)
5′-AGUGGCACATACCACACCCU-3′;
(SEQ ID NO: 234)
5′-AAGAUTATCTTCTTTUAAUU-3′;
(SEQ ID NO: 22)
5′-AGAAAGAAGCAAAGAUUCAA-3′;
(SEQ ID NO: 235)
5′-UCCCATCCCTTCTGCUGCCA-3′;
(SEQ ID NO: 156)
5′-ACCCUTCTCTCTGGTCCCAU-3′;
(SEQ ID NO: 236)
5′-AAUAUCTGCTGCCCACCUUC-3′;
(SEQ ID NO: 237)
5′-UCUCUCTGGTCCCATCCCUU-3′;
(SEQ ID NO: 238)
5′-AGGCCTCCAGTGTCTUCUCC-3′;
(SEQ ID NO: 239)
5′-CAAGCCCCAGCGTTCCUCCG-3′;
(SEQ ID NO: 162)
5′-AAAUGTCCTGGCCCTCACUG-3′;
(SEQ ID NO: 166)
5′-AAUUUAAAGCATGAAUAUUA-3′;
(SEQ ID NO: 24)
5′-AAAAGATTATCTTCTUUUAA-3′;
(SEQ ID NO: 144)
5′-AUAUCTGCTGCCCACCUUCU-3′;
(SEQ ID NO: 240)
5′-CAGUCTCCATGTCCCAGGCC-3′;
(SEQ ID NO: 241)
5′-AAAGATTATCTTCTTUUAAU-3′;
(SEQ ID NO: 155)
5′-CCCUUCTCTCTGGTCCCAUC-3′;
(SEQ ID NO: 9)
5′-AUGGCCTTTCCGTGCCAAGG-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 242)
5′-GGGUATCGAAAGAGTCUGGA-3′;
(SEQ ID NO: 243)
5′-CUUGCACGTGGCTTCGUCUC-3′;
(SEQ ID NO: 244)
5′-GUGUCCTTGCACGTGGCUUC-3′;
(SEQ ID NO: 245)
5′-GUAAAAAGCTTTTGAAGUGA-3′;
(SEQ ID NO: 246)
5′-AUGCCATCCACTTGAUAGGC-3′;
(SEQ ID NO: 247)
5′-UGAAGTAAAAATCAAUAGCG-3′;
(SEQ ID NO: 248)
5′-AAGGCCCTTCGCACTUCUUA-3′;
(SEQ ID NO: 249)
5′-GUACUCGTCGGCATCCACCA-3′;
(SEQ ID NO: 250)
5′-GUCCUTGCACGTGGCUUCGU-3′;
(SEQ ID NO: 251)
5′-GCCCATCCATGAGGTCCUGG-3′;
(SEQ ID NO: 252)
5′-GUAAAAGGAGAAAACUAUCU-3′;
(SEQ ID NO: 253)
5′-UUGAAGTGAAGTAAAAGGAG-3′;
(SEQ ID NO: 254)
5′-GUUACTCGTGCCTTGGCAAA-3′;
(SEQ ID NO: 255)
5′-GUCCAAGATCAGCAGUCUCA-3′;
(SEQ ID NO: 256)
5′-UUCAATGGGAGAATAAAGCA-3′;
(SEQ ID NO: 257)
5′-GCAAGGCCCTTCGCACUUCU-3′;
(SEQ ID NO: 258)
5′-GGGUCCACCACTAGCCAGUA-3′;
(SEQ ID NO: 259)
5′-GUAGAGAAATTATTTUAGGA-3′;
(SEQ ID NO: 260)
5′-AUCCACCACGTCGTCCAUGU-3′;
(SEQ ID NO: 261)
5′-GGCAUCCACCACGTCGUCCA-3′;
(SEQ ID NO: 262)
5′-UUACUTTAAAAGCAAAAGGA-3′;
(SEQ ID NO: 263)
5′-UUUGAAGTGAAGTAAAAGGA-3′;
(SEQ ID NO: 264)
5′-GAAGUGAAGTAAAAGGAGAA-3′;
(SEQ ID NO: 265)
5′-GGCCATCTCTGCTTCUUGGU-3′;
(SEQ ID NO: 266)
5′-UGGGCTGGAATCCGAGUUAU-3′;
(SEQ ID NO: 267)
5′-GGAGATTTCAGAGCAGCUUC-3′;
(SEQ ID NO: 268)
5′-UUUACGGTTTTCAGAAUAUC-3′;
(SEQ ID NO: 269)
5′-GCGUGTCTGGAAGCTUCCUU-3′;
(SEQ ID NO: 270)
5′-GCUUATTTTAAGCATAUUAA-3′;
(SEQ ID NO: 271)
5′-UUAUUTTAAGCATATUAAAA-3′;
(SEQ ID NO: 272)
5′-UUCUGCAGCTTCCTTGUCCU-3′;
(SEQ ID NO: 273)
5′-AUUACTTTAAAAGCAAAAGG-3′;
(SEQ ID NO: 274)
5′-AUUUUAAGCATATTAAAAAG-3′;
(SEQ ID NO: 275)
5′-UGUGGCTTGTCCTCAGACAU-3′;
(SEQ ID NO: 276)
5′-AAAAGGAGAAAACTAUCUUC-3′;
(SEQ ID NO: 277)
5′-GGGUCCATACCCAAGGCAUC-3′;
(SEQ ID NO: 278)
5′-ACAGUGTTGAGATACUCGGG-3′;
(SEQ ID NO: 279)
5′-GGAUCTGCATGCCCTCAUCU-3′;
(SEQ ID NO: 280)
5′-AGUAAAAAGCTTTTGAAGUG-3′;
(SEQ ID NO: 281)
5′-GUCGUGGCAAATAGTCCUAG-3′;
(SEQ ID NO: 282)
5′-GGAGATCAGATGAGAGGAGC-3′;
(SEQ ID NO: 283)
5′-GUGGUTAAGTACATGAGCUC-3′;
(SEQ ID NO: 284)
5′-GGACACTTAGCTGTTCCUCG-3′;
(SEQ ID NO: 285)
5′-GUCUCTACTGTTACCUCUGA-3′;
(SEQ ID NO: 286)
5′-GAGUUCTTCGTAGGCUUCUG-3′;
(SEQ ID NO: 287)
5′-AAAGUCAAAAAGAAAAACUG-3′;
(SEQ ID NO: 288)
5′-AAAAGTGGGAAATAAAGGUU-3′;
(SEQ ID NO: 289)
5′-AGUUUATAGATTTCAAGUAG-3′;
(SEQ ID NO: 290)
5′-AAAAAGTGGGAAATAAAGGU-3′;
(SEQ ID NO: 291)
5′-UUUAUATTACAAAGCUACUU-3′;
(SEQ ID NO: 292)
5′-UGCUATTCATATTTTUAUUU-3′;
(SEQ ID NO: 56)
5′-GGUAU-3′;
(SEQ ID NO: 293)
5′-[G/A/C][G/A][G/A/C][T/A/C]TC-3′;
(SEQ ID NO: 294)
5′-[G/A/C]G[G/A/C][T/A/C]TC-3′;
(SEQ ID NO: 295)
5′-GGCTTC-3′;
(SEQ ID NO: 296)
5′-GGCATC-3′;
(SEQ ID NO: 297)
5′-AGCTTC-3′;
(SEQ ID NO: 298)
5′-GGAATC-3′;
(SEQ ID NO: 299)
5′-CACATC-3′;
(SEQ ID NO: 204)
5′-GGCCTC-3′;
(SEQ ID NO: 300)
5′-CACTTC-3′;
(SEQ ID NO: 301)
5′-AAGATC-3′;
(SEQ ID NO: 302)
5′-TGTCCTTGCACGTGGCTTCG-3′;
(SEQ ID NO: 94)
5′-TTTGCACACTTCGTACCCAA-3′;
(SEQ ID NO: 303)
5′-GTCCACATCCTGTGGCTCGT-3′;
(SEQ ID NO: 304)
5′-TGTGATGGCCTCCCATCTCC-3′;
(SEQ ID NO: 175)
5′-GGTTTTGGCTGGGATCAAGT-3′;
(SEQ ID NO: 93)
5′-GGTGTCCTTGCACGTGGCTT-3′;
(SEQ ID NO: 305)
5′-GGTCCATACCCAAGGCATCC-3′;
(SEQ ID NO: 306)
5′-GTGTCTTCATCGGCCCTGCC-3′;
(SEQ ID NO: 89)
5′-GTCTTGGCTTCGTGGAGCAG-3′;
(SEQ ID NO: 95)
5′-GCTGACAAAGATTCACTGGT-3′;
(SEQ ID NO: 103)
5′-GAAAGGTTATGCAAGG-3′;
(SEQ ID NO: 307)
5′-GACTATACGCGCAATA-3′;
(SEQ ID NO: 308)
5′-TGTGATGGCCTCCCAT-3′;
(SEQ ID NO: 114)
5′-TCCAACACTTCGTGGG-3′;
(SEQ ID NO: 106)
5′-GTGTCTGGAAGCTTCC-3′;
(SEQ ID NO: 98)
5′-CTTGAAGCATCGTATC-3′;
(SEQ ID NO: 309)
5′-TCGTAGTTGCTTCCTA-3′;
(SEQ ID NO: 105)
5′-CGCTTTTCTGTCTGGT-3′;
(SEQ ID NO: 310)
5′-GGCTGGAATCCGAGTT-3′;
(SEQ ID NO: 100)
5′-GATAGCACCTTCAGCA-3′;
(SEQ ID NO: 311)
5′-AGGACTCCAGATGTTT-3′;
(SEQ ID NO: 312)
5′-GTGATCTTGACATGCT-3′;
(SEQ ID NO: 313)
5′-AGATTTCAGAGCAGCT-3′;
(SEQ ID NO: 314)
5′-GGTTACGGCTCAGTAT-3′;
(SEQ ID NO: 315)
5′-GTTCAGTCCTGTCCAT-3′;
(SEQ ID NO: 316)
5′-AGGTCTTGGCTTCGTG-3′;
(SEQ ID NO: 191)
5′-CTGCAGCTTCCTTGTC-3′;
(SEQ ID NO: 317)
5′-GTCCTTGCACGTGGCT-3′;
(SEQ ID NO: 318)
5′-GTCTCTGGAGCTTCCT-3′;
(SEQ ID NO: 319)
5′-GGTCTTGGCTTCGTGG-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
5′-GUC-3′;
5′-GUG-3′;
5′-GUU-3′;
5′-GGC-3′;
5′-AUC-3′;
5′-GAG-3′;
5′-GGA-3′;
5′-TTT-3′;
5′-TCT-3′;
5′-GAA-3′;
5′-GAC-3′;
5′-GAU-3′;
5′-AUG-3′;
5′-GCG-3′;
5′-UUC-3′;
5′-GCC-3′;
5′-GGG-3′;
5′-AUU-3′;
5′-GCA-3′;
5′-AGC-3′;
5′-AAC-3′;
5′-CCA-3′;
5′-UGC-3′;
5′-CAA-3′;
5′-CGG-3′;
5′-ACC-3′;
5′-AGA-3′;
5′-TTT-3′;
5′-TCT-3′;

and
a variant of the motifs or sequences thereof having at least about 75% sequence identity thereto;
wherein the U may be a T and/or the T may be a U.

In one embodiment, the step of modifying the oligonucleotide includes adding a sequence of nucleotides to the 5′ and/or 3′ end of the oligonucleotide such that the modified oligonucleotide comprises the motif

In particular embodiments, the step of modifying the oligonucleotide includes adding the motif selected from 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) and 5′-GUA-3′ (SEQ ID NO: 60), 5′-GUC-3′, 5′-GUG-3′, 5′-GUU-3′, 5′-GGC-3′, 5′-AUC-3′, 5′-GAA-3′, 5′-GAG-3′, 5′-GGA-3′, 5′-GAC-3′, 5′-GAU-3′, 5′-AUG-3′, 5′-GCG-3′, 5′-UUC-3′, 5′-GCC-3′, 5′-GGG-3′, 5′-AUU-3′, 5′-GCA-3′, 5′-AGC-3′, 5′-AAC-3′, 5′-CCA-3′, 5′-UGC-3′, 5′-CAA-3′, 5′-CGG-3′, 5′-ACC-3′, 5′-AGA-3′, 5′-TTT-3′, or 5′-TCT-3′ or a portion or fragment thereof, wherein the U may be a T, to the 5′ and/or 3′ end of the oligonucleotide, such that the modified oligonucleotide comprises the motif. More particularly, the step of modifying the oligonucleotide suitably includes adding the motif selected from 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59), 5′-GUA-3′ (SEQ ID NO: 60), 5′-GUC-3′, 5′-GUG-3′, 5′-GUU-3′, 5′-GUA-3′, 5′-GGC-3′, 5′-AUC-3′, 5′-GAA-3′, 5′-GAG-3′, 5′-GGA-3′, 5′-GAC-3′, 5′-GAU-3′, 5′-AUG-3′, 5′-GCG-3′, 5′-UUC-3′, 5′-GCC-3′, 5′-GGG-3′, 5′-AUU-3′, 5′-GCA-3′, 5′-AGC-3′, 5′-AAC-3′, 5′-CCA-3′, 5′-UGC-3′, 5′-CAA-3′, 5′-CGG-3′, 5′-ACC-3′, 5′-AGA-3′, 5′-TTT-3′ or 5′-TCT-3′ wherein the U may be a T, to the 5′ end of the oligonucleotide, such that the modified oligonucleotide comprises the motif.

In another embodiment, the present method further comprises testing the ability of the modified oligonucleotide to inhibit TLR7 activity, and selecting an oligonucleotide which inhibits TLR7 activity to a greater extent than the unmodified oligonucleotide.

Suitably, for the two above aspects the oligonucleotide does not bind or is not designed to bind a transcript that encodes TLR7 or a complement thereof.

Suitably, for the two above aspects the oligonucleotide binds or is designed to bind a target transcript that does not encode TLR7 or a complement thereof.

In an alternative embodiment of the above aspects, the oligonucleotide binds or is designed to bind a target transcript that encodes TLR7 or a complement thereof.

In one embodiment of the above two aspects, the motif is within eleven bases of the 5′ and/or 3′ end of the oligonucleotide.

In one embodiment of the above two aspects, the motif is within eight bases of the 5′ and/or 3′ end of the oligonucleotide.

In one embodiment of the above two aspects, the motif is at or towards the 5′ and/or 3′ end of the oligonucleotide.

In one embodiment of the above two aspects, the motif has the sequence 5′-[A/G]GU[A/C][U/C]C-3′ (SEQ ID NO: 1); 5′-A[G/A][U/G]C[U/C]C-3′ (SEQ ID NO: 2); 5′-A[G/A][U/G]C[U/C]C[U/C][C/A]U-3′ (SEQ ID NO: 212); 5′-GGUAUA-3′ (SEQ ID NO: 4); 5′-UGUUUC-3′ (SEQ ID NO: 5); 5′-UGUGUC-3′ (SEQ ID NO: 6); 5′-CGUGUC-3′ (SEQ ID NO: 8); 5′-GCAGUCTCCATGTCCCAGGC-3′ (SEQ ID NO: 30); 5′-GAUGGTTCCAGTCCCUCUUC-3′ (SEQ ID NO: 38); 5′-AGCAGTCTCCATGTCCCAGG-3′ (SEQ ID NO: 31); 5′-GGGUCTCCTCCACACCCUUC-3′ (SEQ ID NO: 36); 5′-GGUGGCCACAGGCAACGUCA-3′ (SEQ ID NO: 28); 5′-GCCGUTTCCATGTCCCAGGC-3′ (SEQ ID NO: 46); 5′-GCGGUATCCATGTCCCAGGC-3′ (SEQ ID NO: 42); 5′-GCGGUATACAGGTCCCAGGC-3′ (SEQ ID NO: 43); 5′-GCUGUTTCCATGTCCCAGGC-3′ (SEQ ID NO: 44); 5′-GCUGUGTCCATGTCCCAGGC-3′ (SEQ ID NO: 45); 5′-GCCGUGTCCATGTCCCAGGC-3′ (SEQ ID NO: 47); 5′-GCGGUAUCCAUGUCCCAGGC-3′ (SEQ ID NO: 151); 5′-GGUATCCCCCCCCCCCCCCC-3′ (SEQ ID NO: 54); 5′-GUCCCATCCCTTCTGCUGCC-3′ (SEQ ID NO: 145); 5′-UUCUCTCTGGTCCCAUCCCU-3′ (SEQ ID NO: 213); 5′-GUUCAGTCAGATCGCUGGGA-3′ (SEQ ID NO: 214); 5′-AUGACATTTCGTGGCUCCUA-3′ (SEQ ID NO: 215); 5′-UCUCCATGTCCCAGGCCUCC-3′ (SEQ ID NO: 216); 5′-AGUCUCCATGTCCCAGGCCU-3′ (SEQ ID NO: 217); 5′-CCAUGTCCCAGGCCTCCAGU-3′ (SEQ ID NO: 29); 5′-GCAAGGCAGAGAAACUCCAG-3′ (SEQ ID NO: 37); 5′-GGAUUAAAACAGATTAAUAC-3′ (SEQ ID NO: 55); 5′-AGCCGAACAGAAGGAGCGUC-3′ (SEQ ID NO: 40); 5′-UCCGGCCTCGGAAGCUCUCU-3′ (SEQ ID NO: 10); 5′-UCCGGCCTCGGAGTCUCCAU-3′ (SEQ ID NO: 52); 5′-GCGGUATCCATAGTCUCCAU-3′ (SEQ ID NO: 48); 5′-GAUUAAAACAGATTAAUACA-3′ (SEQ ID NO: 165); 5′-UGACAAAACAATAATAACAG-3′ (SEQ ID NO: 167); or 5′-CCAACACTTCGTGGGGUCCU-3′ (SEQ ID NO: 160), wherein the U may be a T and/or the T may be a U.

In one embodiment of the above two aspects, the motif has the sequence: 5′-GGCATCCACCACGTCGTCCA-3′ (SEQ ID NO: 320); 5′-GTCCTTGCACGTGGCTTCGT-3′ (SEQ ID NO: 321); 5′-TGTCCTTGCACGTGGCTTCG-3′ (SEQ ID NO: 302); 5′-GGAGATTTCAGAGCAGCTTC-3′ (SEQ ID NO: 322); 5′-TTCTGCAGCTTCCTTGTCCT-3′ (SEQ ID NO: 323); 5′-TGGGCTGGAATCCGAGTTAT-3′ (SEQ ID NO: 179); 5′-GTCCACATCCTGTGGCTCGT-3′ (SEQ ID NO: 303); 5′-TGTGATGGCCTCCCATCTCC-3′ (SEQ ID NO: 304); 5′-TTTGCACACTTCGTACCCAA-3′ (SEQ ID NO: 94); or 5′-GTCCAAGATCAGCAGTCTCA (SEQ ID NO: 178), wherein the U may be a T and/or the T may be a U.

In particular embodiments of the above two aspects, the motif has the sequence:

5′-mGmGmCmAmTCCACCACGTCmGmTmCmCmA-3′;
5′-mGmTmCmCmTTGCACGTGGCmTmTmCmGmT-3′;
5′-mTmGmTmCmCTTGCACGTGGmCmTmTmCmG-3′;
5′-mGmGmAmGmATTTCAGAGCAmGmCmTmTmC-3′;
5′-mTmTmCmTmGCAGCTTCCTTmGmTmCmCmT-3′;
5′-mTmGmGmGmCTGGAATCCGAmGmTmTmAmT-3′;
5′-mGmTmCmCmACATCCTGTGGmCmTmCmGmT-3′;
5′-mTmGmTmGmATGGCCTCCCAmTmCmTmCmC-3′;
5′-mTmTmTmGmCACACTTCGTAmCmCmCmAmA-3′;
or
5′-mGmTmCmCmAAGATCAGCAGmTmCmTmCmA-3′;

wherein the U may be a T and/or the T may be a U and wherein m is a modified base and/or has a modified backbone. For such examples, m is suitably a 2′-MOE modified base.

In one embodiment of the above two aspects, the motif has the sequence 5′-GGUAUC-3′ (SEQ ID NO: 120), 5′-AGUCUC-3′ (SEQ ID NO: 121), 5′-GGUCCC-3′ (SEQ ID NO: 122), 5′-GGUCUC-3′ (SEQ ID NO: 123), 5′-AAGCUC-3′ (SEQ ID NO: 124), 5′-AGUCCC-3′ (SEQ ID NO: 125), 5′-GGUAUA-3′ (SEQ ID NO: 4), 5′-UGUUUC-3′ (SEQ ID NO: 5), 5′-UGUGUC-3′ (SEQ ID NO: 6), 5′-CGUUUC-3′ (SEQ ID NO: 7), 5′-CGUGUC-3′ (SEQ ID NO: 8), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60),wherein the U may be a T.

In one embodiment of the above two aspects, the motif has the sequence of 5′-GGUAUC-3′ (SEQ ID NO: 120), 5′-GGUATC-3′ (SEQ ID NO: 119), 5′-AGUCTC-3′ (SEQ ID NO: 126), 5′-AGTCTC-3′ (SEQ ID NO: 127), 5′-GGUCCC-3′ (SEQ ID NO: 122), 5′-GGUCTC-3′ (SEQ ID NO: 128), 5′-AAGCUC-3′ (SEQ ID NO: 124), 5′-AGTCCC-3′ (SEQ ID NO: 129), 5′-GGUATA-3′ (SEQ ID NO: 130), 5′-UGUTTC-3′ (SEQ ID NO: 131), 5′-UGUGTC-3′ (SEQ ID NO: 132), 5′-CGUTTC-3′ (SEQ ID NO: 133), 5′-CGUGTC-3′ (SEQ ID NO: 134), 5′-GGUAT-3′ (SEQ ID NO: 135), 5′-GGUAU-3′ (SEQ ID NO: 56), 5′-GGUA-3′ (SEQ ID NO: 57), 5′-GUAU-3′ (SEQ ID NO: 58), 5′-GUAT-3′ (SEQ ID NO: 136), 5′-GGU-3′ (SEQ ID NO: 59) or 5′-GUA-3′ (SEQ ID NO: 60), 5′-GUC-3′, 5′-GUG-3′, 5′-GUU-3′, 5′-GGC-3′, 5′-AUC-3′, 5′-GAA-3′, 5′-GAG-3′, 5′-GGA-3′, 5′-GAC-3′, 5′-GAU-3′, 5′-AUG-3′, 5′-GCG-3′, 5′-UUC-3′, 5′-GCC-3′, 5′-GGG-3′, 5′-AUU-3′, 5′-GCA-3′, 5′-AGC-3′, 5′-AAC-3′, 5′-CCA-3′, 5′-UGC-3′, 5′-CAA-3′, 5′-CGG-3′, 5′-ACC-3′, 5′-AGA-3′, 5′-TTT-3′ or 5′-TCT-3′.

In one embodiment, the motif has the sequence of 5′-GGUAU-3′ (SEQ ID NO: 56), wherein the U may be a T and wherein the motif is at or towards a 5′ end of the oligonucleotide.

In a further embodiment, the motif has the sequence of 5′-GGUA-3′ (SEQ ID NO: 57), wherein the U may be a T and wherein the motif is at or towards a 5′ end of the oligonucleotide.

In a related embodiment, the motif has the sequence of 5′-GUAU-3′ (SEQ ID NO: 58), wherein the U may be a T and wherein the motif is at or towards a 5′ end of the oligonucleotide.

In another embodiment, the motif has the sequence of 5′-GGU-3′ (SEQ ID NO: 59), wherein the U may be a T and wherein the motif is at or towards a 5′ end of the oligonucleotide.

In one embodiment, the motif has the sequence of 5′-GUA-3′ (SEQ ID NO: 60), wherein the U may be a T and wherein the motif is at or towards a 5′ end of the oligonucleotide.

In one embodiment of the above two aspects, one or more of the bases of the motif are a modified base and/or have a modified backbone.

In one embodiment of the above two aspects, the motif has the sequence of 5′-mGmGmUATC-3′, 5′-mAmGmUCTC-3′, 5′-mAmGTCTC-3′, 5′-mGmGmUmCmCC-3′, 5′-mGmGmUmCTC-3′, 5′-AAGCmUmC-3′, 5′-AGTCCC-3′ (SEQ ID NO: 129), 5′-mGmGmUATA-3′, 5′-mUmGmUTTC-3′, 5′-mUmGmUGTC-3′, 5′-mCmGmUTTC-3′, 5′-mCmGmUGTC-3′, 5′-mGmGmUAU-3′, 5′-mGmGmUAT-3′, 5′-mGmGmUmAU-3′, 5′-mGmGmUmAT-3′, 5′-mGmGmUmAmU-3′, 5′-mGmGmUA-3′, 5′-mGmGmUmA-3′, 5′-mGmUmAmU-3′, 5′-mGmGmU-3′ or 5′-mGmUmA-3′, wherein m is a modified base and/or has a modified backbone.

In any embodiment of the above aspects, the motif comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR7.

With respect to the above three aspects, the method may include the further step of testing the ability of the one or more candidate oligonucleotides or the modified oligonucleotide to inhibit TLR3, TLR8, TLR9 and/or cGAS activity, and optionally selecting an oligonucleotide which inhibits or does not substantially inhibit TLR3, TLR8, TLR9 and/or cGAS activity.

In particular embodiments, the one or more candidate oligonucleotides or the modified oligonucleotide inhibit TLR7 and TLR9 activity. Examples of the motif of such embodiments include 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52), 5′-GCGGUATCCATAGTCUCCAU-3′ (SEQ ID NO: 48), 5′-GAUUAAAACAGATTAAUACA-3′ (SEQ ID NO: 165), 5′-UGACAAAACAATAATAACAG-3′ (SEQ ID NO: 167) and 5′-CCAACACTTCGTGGGGUCCU-3′ (SEQ ID NO: 160).

In particular embodiments, the one or more candidate oligonucleotides or the modified oligonucleotide inhibit TLR7 and TLR9 activity, but do not substantially inhibit cGAS activity. Examples of the motif of such embodiments include 5′-GAUUAAAACAGATTAAUACA-3′ (SEQ ID NO: 165) and 5′-UGACAAAACAATAATAACAG-3′ (SEQ ID NO: 167).

In particular embodiments, the one or more candidate oligonucleotides or the modified oligonucleotide inhibit TLR7 and cGAS activity. Examples of the motif of such embodiments include 5′-GCAGUCTCCATGTCCCAGGC-3′ (SEQ ID NO: 30), 5′-GAUGGTTCCAGTCCCUCUUC-3′ (SEQ ID NO: 38), 5′-AGCAGTCTCCATGTCCCAGG-3′ (SEQ ID NO: 31), 5′-GGGUCTCCTCCACACCCUUC-3′ (SEQ ID NO: 36), 5′-GGUGGCCACAGGCAACGUCA-3′ (SEQ ID NO: 28), 5′-GCCGUTTCCATGTCCCAGGC-3′ (SEQ ID NO: 46), 5′-GCGGUATCCATGTCCCAGGC-3′ (SEQ ID NO: 42), 5′-GCGGUATACAGGTCCCAGGC-3′ (SEQ ID NO: 43), 5′-GCUGUTTCCATGTCCCAGGC-3′ (SEQ ID NO: 44), 5′-GCUGUGTCCATGTCCCAGGC-3′ (SEQ ID NO: 45), 5′-GCCGUGTCCATGTCCCAGGC-3′ (SEQ ID NO: 47), 5′-GCGGUAUCCAUGUCCCAGGC-3′ (SEQ ID NO: 151), 5′-GGUATCCCCCCCCCCCCCCC-3′ (SEQ ID NO: 54), 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52), 5′-GCGGUATCCATAGTCUCCAU-3′ (SEQ ID NO: 48), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) and 5′-GUA-3′ (SEQ ID NO: 60).

In further embodiments, the one or more candidate oligonucleotides or the modified oligonucleotide inhibit TLR7 and cGAS activity, but do not substantially inhibit TLR9 activity. Examples of the motif of such embodiments include 5′-GCAGUCTCCATGTCCCAGGC-3′ (SEQ ID NO: 30), 5′-GAUGGTTCCAGTCCCUCUUC-3′ (SEQ ID NO: 38), 5′-AGCAGTCTCCATGTCCCAGG-3′ (SEQ ID NO: 31), 5′-GGGUCTCCTCCACACCCUUC-3′ (SEQ ID NO: 36), 5′-GGUGGCCACAGGCAACGUCA-3′ (SEQ ID NO: 28), 5′-GCCGUTTCCATGTCCCAGGC-3′ (SEQ ID NO: 46), 5′-GCGGUATCCATGTCCCAGGC-3′ (SEQ ID NO: 42), 5′-GCGGUATACAGGTCCCAGGC-3′ (SEQ ID NO: 43), 5′-GCUGUTTCCATGTCCCAGGC-3′ (SEQ ID NO: 44), 5′-GCUGUGTCCATGTCCCAGGC-3′ (SEQ ID NO: 45), 5′-GCCGUGTCCATGTCCCAGGC-3′ (SEQ ID NO: 47), 5′-GCGGUAUCCAUGUCCCAGGC-3′ (SEQ ID NO: 151) and 5′-GGUATCCCCCCCCCCCCCCC-3′ (SEQ ID NO: 54), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) and 5′-GUA-3′ (SEQ ID NO: 60).

In further embodiments, the one or more candidate oligonucleotides or the modified oligonucleotide inhibit TLR7, TLR9 and cGAS activity. Examples of the motif of such embodiments include 5′-CCAUGTCCCAGGCCTCCAGU-3′ (SEQ ID NO: 29), 5′-GCAAGGCAGAGAAACUCCAG-3′ (SEQ ID NO: 37), 5′-GGAUUAAAACAGATTAAUAC-3′ (SEQ ID NO: 55), 5′-AGCCGAACAGAAGGAGCGUC-3′ (SEQ ID NO: 40), 5′-UCCGGCCTCGGAAGCUCUCU-3′ (SEQ ID NO: 10), 5′-UCCGGCCTCGGAGTCUCCAU-3′ (SEQ ID NO: 52) and 5′-GCGGUATCCATAGTCUCCAU-3′ (SEQ ID NO: 48).

In alternative embodiments, the one or more candidate oligonucleotides or the modified oligonucleotide inhibit TLR7, but do not substantially inhibit TLR9 and/or cGAS. Examples of the motif of such embodiments include 5′-GUCCCATCCCTTCTGCUGCC-3′ (SEQ ID NO: 145), 5′-UUCUCTCTGGTCCCAUCCCU-3′ (SEQ ID NO: 213), 5′-GUUCAGTCAGATCGCUGGGA-3′ (SEQ ID NO: 214), 5′-AUGACATTTCGTGGCUCCUA-3′ (SEQ ID NO: 215), 5′-UCUCCATGTCCCAGGCCUCC-3′ (SEQ ID NO: 216) and 5′-AGUCUCCATGTCCCAGGCCU-3′ (SEQ ID NO: 217).

With respect to the above two aspects, the method may include the further step of testing the ability of the one or more candidate oligonucleotides or the modified oligonucleotide to potentiate TLR8 activity, and optionally selecting an oligonucleotide which potentiates or does not substantially potentiate TLR8 activity. Accordingly, in some embodiments, the one or more candidate oligonucleotides or the modified oligonucleotide inhibit TLR7 activity and potentiate TLR8 activity. In other embodiments, the one or more candidate oligonucleotides or the modified oligonucleotide inhibit TLR7 activity and do not substantially potentiate TLR8 activity.

In yet another aspect, the invention relates to a method for selecting or designing an oligonucleotide which increases or potentiates TLR8 activity, the method comprising i) scanning a polynucleotide, or complement thereof, for a region having a motif including a sequence selected from the group consisting of:

5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′;
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;

a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to increase or potentiate TLR8 activity, and
iv) selecting an oligonucleotide which increases or potentiate TLR8 activity.

In still another aspect, the invention resides in a method for increasing or potentiating the TLR8 activity of an oligonucleotide, or increasing the potentiating activity of an oligonucleotide, the method comprising modifying the oligonucleotide such that the modified oligonucleotide comprises a motif including a sequence selected from the group consisting of:

5′-CUUCGTGGGGTCCTTUUCAC-3′;
5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′:
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;

and
a variant of the motifs or sequences thereof having at least about 75% sequence identity thereto;
wherein the U may be a T and/or the T may be a U.

In one embodiment, the step of modifying the oligonucleotide includes adding a sequence of nucleotides to the 5′ and/or 3′ end of the oligonucleotide such that the modified oligonucleotide comprises the motif.

In particular embodiments, the step of modifying the oligonucleotide includes adding the motif selected from 5′-CUUCG-3′, 5′-CUUCGTG-3′, 5′-CUUCGTGGG-3′, 5′-UCG-3, 5′-CGG-3′, 5′-UGG-3′, 5′-CGC-3′, 5′-AGG-3′, 5′-GGA-3′, 5′-GGC-3′; 5′-AGA-3′; 5′-CGA-3′; 5′-UAG-3′; 5′-UCU-3′; 5′-AGC-3′; 5′-GGU-3′; 5′-UGA-3′; 5′-AGU-3′; 5′-ACG-3′; 5′-CGU-3′; 5′-UCC-3′; 5′-GCG-3′; 5′-GGG-3′; 5′-UGU-3′; 5′-UCA-3′; 5′-CUG-3′; 5′-UUG-3′; 5′-UUA-3′ and 5′-UGC-3′ or a portion or fragment thereof, wherein the U may be a T, to the 5′ and/or 3′ end of the oligonucleotide, such that the modified oligonucleotide comprises the motif More particularly, the step of modifying the oligonucleotide suitably includes adding the motif selected from 5′-CUUCG-3′, 5′-CUUCGTG-3′, 5′-CUUCGTGGG-3′, 5′-UCG-3, 5′-CGG-3′, 5′-UGG-3′, 5′-CGC-3′, 5′-AGG-3′, 5′-GGA-3′, 5′-GGC-3′; 5′-AGA-3′; 5′-CGA-3′; 5′-UAG-3′; 5′-UCU-3′; 5′-AGC-3′; 5′-GGU-3′; 5′-UGA-3′; 5′-AGU-3′; 5′-ACG-3′; 5′-CGU-3′; 5′-UCC-3′; 5′-GCG-3′; 5′-GGG-3′; 5′-UGU-3′; 5′-UCA-3′; 5′-CUG-3′; 5′-UUG-3′; 5′-UUA-3′ and 5′-UGC-3′ wherein the U may be a T, to the 5′ end of the oligonucleotide, such that the modified oligonucleotide comprises the motif.

In another embodiment, the present method further comprises testing the ability of the modified oligonucleotide to increase or potentiate TLR8 activity, and selecting an oligonucleotide which increases or potentiates TLR8 activity to a greater extent than the unmodified oligonucleotide.

Suitably, for the two above aspects the oligonucleotide does not bind or is not designed to bind a transcript that encodes TLR8 or a complement thereof.

Suitably, for the two above aspects the oligonucleotide binds or is designed to bind a target transcript that does not encode TLR8 or a complement thereof.

In an alternative embodiment of the above aspects, the oligonucleotide binds or is designed to bind a target transcript that encodes TLR8 or a complement thereof.

In one embodiment of the above two aspects, the motif is within eleven bases of the 5′ and/or 3′ end of the oligonucleotide.

In one embodiment of the above two aspects, the motif is within eight bases of the 5′ and/or 3′ end of the oligonucleotide.

In one embodiment of the above two aspects, the motif is at or towards the 5′ and/or 3′ end of the oligonucleotide.

In one embodiment of the above two aspects, the motif has the sequence 5′-CUUCG-3′, 5′-CUUCGTG-3′, 5′-CUUCGTGGG-3′, 5′-UCG-3, 5′-CGG-3′, 5′-UGG-3′, 5′-CGC-3′, 5′-AGG-3′ and 5′-GGA-3′, wherein the U may be a T and/or the T may be a U.

In particular embodiments of the above two aspects, the motif has the sequence:

5′-CUUCG-3′,
5′-CUUCGTG-3′,
5′-CUUCGTGGG-3′,
5′-UCG-3′,
5′-CGG-3′,
5′-UGG-3′,
5′-CGC-3′,
5′-AGG-3′,
or
5′-GGA-3′,

wherein one, two or more bases are a modified base and/or one or more internucleotide linkages has a modified backbone, preferably wherein the modified base is 2′OMe and the modified backbone is phosphorothioate.

In particular embodiments of the above two aspects, the motif has the sequence:

5′-mCmUmUmCmG-3′,
5′-mCmUmUmCmGTG-3′,
5′-mCmUmUmCmGTGGG-3′,
5′-mUmCmG-3′,
5′-mCmGmG-3′,
5′-mUmGmG-3′,
5′-mCmGmC-3′,
5′-mAmGmG-3′,
or
5′-mGmGmA-3′,

wherein m is a modified base and/or has a modified backbone, preferably wherein the modified base is 2′OMe and the modified backbone is phosphorothioate.

In particular embodiments of the above two aspects, the motif has the sequence:

5′-mC*mU*mU*C*G*T*G*G*G*G*T*C*C*T*T*mU*mU*mC*
mA*mC-3′;
5′-mC*mU*mU*mC*mG-3′,
5′-mC*mU*mU*mC*mG*T*G-3′,
5′-mC*mU*mU*mC*mG*T*G*G*G-3′,
5′-mU*mC*mG*-3′,
5′-mC*mG*mG*-3′,
5′-mU*mG*mG*-3′,
5′-mC*mG*mC*-3′,
5′-mA*mG*mG*-3′,
or
5′-mG*mG*mA*- ′,

wherein m is a 2′OMe modified base, T/G are DNA bases and * is a phosphorothioate modified backbone.

In one embodiment of the above two aspects, one or more of the bases of the motif are a modified base and/or have a modified backbone. In one embodiment, not every base is modified, for example one or more bases may be unmodified and one or more bases may be modified, for example modified with a 2′OMe.

In any embodiment of the above aspects, the motif comprises, consists essentially of or consists of any sequence in Tables 1, 1A, 2, 2A, 3, 3A, 4, 4A, 5, 5A or 6, with or without the particular modifications defined, that is shown in the Examples to increase or potentiate the activity of TLR8.

With respect to the above three aspects, the method may include the further step of testing the ability of the one or more candidate oligonucleotides or the modified oligonucleotide to inhibit TLR3, TLR7, TLR9 and/or cGAS activity, and optionally selecting an oligonucleotide which inhibits or does not substantially inhibit TLR3, TLR7, TLR9 and/or cGAS activity.

In one embodiment, the candidate oligonucleotides or the modified oligonucleotide comprises, consists essentially of or consists of any sequence in Tables 1, 1A, 2, 2A, 3, 3A, 4, 4A, 5, 5A or 6, with or without the particular modifications defined, that is shown in the Examples to increase or potentiate the activity of TLR8.

In one embodiment, the candidate oligonucleotides or the modified oligonucleotide oligonucleotide comprises, consists essentially of or consists of any sequence in Tables 1, 1A, 2, 2A, 3, 3A, 4, 4A, 5, 5A or 6 that is shown in the Examples to increase or potentiate the activity of TLR8 and the oligonucleotide inhibits, or does not substantially inhibit, TLR3, TLR7, TLR9 and/or cGAS activity.

In yet another aspect, the invention relates to a method for selecting or designing an oligonucleotide which inhibits TLR8 activity, the method comprising
i) scanning a polynucleotide, or complement thereof, for a region having a motif including a sequence selected from the group consisting of:

5′-GAG-3′;
5′-GAC-3′;
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5′-GCU-3′;
5′-CCU-3′;
5′-CUA-3′;
5′-CUC-3′;
5′-AAC-3′;

and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR8 activity, and
iv) selecting an oligonucleotide which inhibits TLR8 activity.

In still another aspect, the invention resides in a method for increasing the TLR8 inhibitory activity of an oligonucleotide, the method comprising modifying the oligonucleotide such that the modified oligonucleotide comprises a motif including a sequence selected from the group consisting of:

5′-GAG-3′;
5′-GAC-3′;
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5′-GCU-3′;
5′-CCU-3′;
5′-CUA-3′;
5′-CUC-3′;
5′-AAC-3′;

and
a variant of the motifs or sequences thereof having at least about 75% sequence identity thereto;
wherein the U may be a T and/or the T may be a U.

In one embodiment, the step of modifying the oligonucleotide includes adding a sequence of nucleotides to the 5′ and/or 3′ end of the oligonucleotide such that the modified oligonucleotide comprises the motif.

In particular embodiments, the step of modifying the oligonucleotide includes adding the motif selected from 5′-GAG-3′; 5′-GAC-3′; 5′-GAU-3′; 5′-GAA-3′; 5′-GUC-3′; 5′-GUU-3′; 5′-GUA-3′; 5′-GUG-3′; 5′-AUA-3′; 5′-AUG-3′; 5′-CUU-3′; 5′-AAG-3′; 5′-AUC-3′; 5′-CCC-3′; 5′-GCU-3′; 5′-CCU-3′; 5′-CUA-3′ and 5′-CUC-3′; 5′-AAC-3′ or a portion or fragment thereof, wherein the U may be a T, to the 5′ and/or 3′ end of the oligonucleotide, such that the modified oligonucleotide comprises the motif. More particularly, the step of modifying the oligonucleotide suitably includes adding the motif selected from 5′-GAG-3′; 5′-GAC-3′; 5′-GAU-3′; 5′-GAA-3′; 5′-GUC-3′; 5′-GUU-3′; 5′-GUA-3′; 5′-GUG-3′; 5′-AUA-3′; 5′-AUG-3′; 5′-CUU-3′; 5′-AAG-3′; 5′-AUC-3′; 5′-CCC-3′; 5′-GCU-3′; 5′-CCU-3′; 5′-CUA-3′; 5′-CUC-3′ and 5′-AAC-3′ wherein the U may be a T, to the 5′ end of the oligonucleotide, such that the modified oligonucleotide comprises the motif.

In another embodiment, the present method further comprises testing the ability of the modified oligonucleotide to inhibit TLR8 activity, and selecting an oligonucleotide which inhibits TLR8 activity to a greater extent than the unmodified oligonucleotide.

Suitably, for the two above aspects the oligonucleotide does not bind or is not designed to bind a transcript that encodes TLR8 or a complement thereof.

Suitably, for the two above aspects the oligonucleotide binds or is designed to bind a target transcript that does not encode TLR8 or a complement thereof.

In an alternative embodiment of the above aspects, the oligonucleotide binds or is designed to bind a target transcript that encodes TLR8 or a complement thereof.

In one embodiment of the above two aspects, the motif is within eleven bases of the 5′ and/or 3′ end of the oligonucleotide.

In one embodiment of the above two aspects, the motif is within eight bases of the 5′ and/or 3′ end of the oligonucleotide.

In one embodiment of the above two aspects, the motif is at or towards the 5′ and/or 3′ end of the oligonucleotide.

In one embodiment of the above two aspects, the motif has the sequence 5′-GAG-3′; 5′-GAC-3′; 5′-GAU-3′; 5′-GAA-3′; 5′-GUC-3′; 5′-GUU-3′; 5′-GUA-3′; 5′-GUG-3′; 5′-AUA-3′; 5′-AUG-3′; 5′-CUU-3′; 5′-AAG-3′; 5′-AUC-3′; 5′-CCC-3′; 5′-GCU-3′; 5′-CCU-3′; 5′-CUA-3′; 5′-CUC-3′; 5′-AAC-3′ wherein the U may be a T and/or the T may be a U.

In particular embodiments of the above two aspects, the motif has the sequence:

5′-GAX-3′
or
5′-GUX-3′ wherein X
is any nucleotide,
5′-GAG-3′,
5′-GAC-3′,
5′-GAU-3′,
5′-GAA-3′,
5′-GUC-3′,
5′-GUU-3′,
5′-GUA-3′,
5′-GUG-3′,

wherein one, two or more bases are a modified base and/or one or more internucleotide linkages has a modified backbone, preferably wherein the modified base is 2′OMe and the modified backbone is phosphorothioate.

In particular embodiments of the above two aspects, the motif has the sequence:

5′-mGmAmX-3′
or
5′-mGmUmX-3′ wherein X
is any nucleotide,
5′-mGmAmG-3′,
5′-mGmAmC-3′,
5′-mGmAmU-3′,
5′-mGmAmA-3′,
5′-mGmUmC-3′,
5′-mGmUmU-3′,
5′-mGmUmA-3′,
5′-mGmUmG-3′.

wherein m is a modified base and/or has a modified backbone, preferably wherein the modified base is 2′OMe and the modified backbone is phosphorothioate.

In one embodiment of the above two aspects, one or more of the bases of the motif are a modified base and/or have a modified backbone. In one embodiment, not every base is modified, for example one or more bases may be unmodified and one or more bases may be modified, for example modified with a 2′OMe.

In any embodiment of the above aspects, the motif comprises, consists essentially of or consists of any sequence in Tables 1, IA, 2, 2A, 3, 3A, 4, 4A, 5, 5A or 6, with or without the particular modifications defined, that is shown in the Examples to inhibit the activity of TLR8.

With respect to the above three aspects, the method may include the further step of testing the ability of the one or more candidate oligonucleotides or the modified oligonucleotide to inhibit TLR3, TLR7, TLR9 and/or cGAS activity, and optionally selecting an oligonucleotide which inhibits or does not substantially inhibit TLR3, TLR7, TLR9 and/or cGAS activity.

In one embodiment, the candidate oligonucleotides or the modified oligonucleotide comprises, consists essentially of or consists of any sequence in Tables 1, 1A, 2, 2A, 3, 3A, 4, 4A, 5, 5A or 6 that is shown in the Examples to inhibit the activity of TLR8.

In yet another aspect, the invention relates to a method for selecting or designing an oligonucleotide which inhibits TLR3 activity, the method comprising
i) scanning a polynucleotide, or complement thereof, for a region having a motif including a sequence selected from the group consisting of:

5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′:
5′-TAG-3′;
5′-TGG-3′;
5′-TCA-3′;
5′-TGC-3′;
5′-CAC-3′:
5′-CGG-3′;
5′-CCC-3′;
5′-ACT-3′;
5′-GTA-3′:
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;

and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR3 activity, and
iv) selecting an oligonucleotide which inhibits TLR3 activity.

In still another aspect, the invention resides in a method for increasing the TLR3 inhibitory activity of an oligonucleotide, the method comprising modifying the oligonucleotide such that the modified oligonucleotide comprises a motif including a sequence selected from the group consisting of:

5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′;
5′-TAG-3′;
5′-TGG-3′;
5′-TCA-3′;
5′-TGC-3′;
5′-CAC-3′;
5′-CGG-3′;
5′-CCC-3′;
5′-ACT-3′;
5′-GTA-3′;
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;

and
a variant of the motifs or sequences thereof having at least about 75% sequence identity thereto;
wherein the U may be a T and/or the T may be a U.

In one embodiment, the step of modifying the oligonucleotide includes adding a sequence of nucleotides to the 5′ and/or 3′ end of the oligonucleotide such that the modified oligonucleotide comprises the motif.

In particular embodiments, the step of modifying the oligonucleotide includes adding the motif selected from 5′-TAC-3′, 5′-CGC-3′, 5′-GCA-3′, 5′-UGA-3′, 5′-CAG-3′, 5′-UGG-3′, 5′-UCA-3′ or a portion or fragment thereof, wherein the U may be a T, to the 5′ and/or 3′ end of the oligonucleotide, such that the modified oligonucleotide comprises the motif More particularly, the step of modifying the oligonucleotide suitably includes adding the motif selected from 5′-TAC-3′, 5′-CGC-3′, 5′-GCA-3′, 5′-UGA-3′, 5′-CAG-3′, 5′-UGG-3′, 5′-UCA-3′ wherein the U may be a T, to the 5′ end of the oligonucleotide, such that the modified oligonucleotide comprises the motif.

In another embodiment, the present method further comprises testing the ability of the modified oligonucleotide to inhibit TLR3 activity, and selecting an oligonucleotide which inhibits TLR3 activity to a greater extent than the unmodified oligonucleotide.

Suitably, for the two above aspects the oligonucleotide does not bind or is not designed to bind a transcript that encodes TLR3 or a complement thereof Suitably, for the two above aspects the oligonucleotide binds or is designed to bind a target transcript that does not encode TLR3 or a complement thereof.

In an alternative embodiment of the above aspects, the oligonucleotide binds or is designed to bind a target transcript that encodes TLR3 or a complement thereof.

In one embodiment of the above two aspects, the motif is within eleven bases of the 5′ and/or 3′ end of the oligonucleotide.

In one embodiment of the above two aspects, the motif is within eight bases of the 5′ and/or 3′ end of the oligonucleotide.

In one embodiment of the above two aspects, the motif is at or towards the 5′ and/or 3′ end of the oligonucleotide.

In one embodiment of the above two aspects, the motif has the 5′-TAC-3′, 5′-CGC-3′, 5′-GCA-3′, 5′-UGA-3′, 5′-CAG-3′, 5′-UGG-3′, 5′-UCA-3′, wherein the U may be a T and/or the T may be a U.

In particular embodiments of the above two aspects, the motif has the sequence:

5′-TAC-3′,
5′-CGC-3′,
5′-GCA-3′,
5′-UGA-3′,
5′-CAG-3′,
5′-UGG-3′,
5′-UCA-3′,

wherein one, two or more bases are a modified base and/or one or more internucleotide linkages has a modified backbone, preferably wherein the modified base is 2′OMe and the modified backbone is phosphorothioate.

In particular embodiments of the above two aspects, the motif has the sequence:

5′-mUmAmC-3′,
5′-mCmGmC-3′,
5′-mGmCmA-3′,
5′-mUmGmA-3′,
5′-mCmAmG-3′,
5′-mUmGmG-3′,
5′-mUmCmA-3′,

and
wherein m is a modified base and/or has a modified backbone, preferably wherein the modified base is 2′OMe and the modified backbone is phosphorothioate.

In one embodiment of the above two aspects, one or more of the bases of the motif are a modified base and/or have a modified backbone.

In any embodiment of the above aspects, the motif comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR3.

With respect to the above three aspects, the method may include the further step of testing the ability of the one or more candidate oligonucleotides or the modified oligonucleotide to inhibit TLR8, TLR7, TLR9 and/or cGAS activity, and optionally selecting an oligonucleotide which inhibits or does not substantially inhibit TLR8, TLR7, TLR9 and/or cGAS activity.

In one embodiment, the candidate oligonucleotides or the modified oligonucleotide comprises, consists essentially of or consists of any sequence in Tables 1, 1A, 2, 2A, 3, 3A, 4, 4A, 5, 5A, 6, or 7 with or without the particular modifications defined, that is shown in the Examples to inhibit the activity of TLR3.

In a further aspect, the invention resides in an oligonucleotide selected, designed or modified using the method of the aforementioned aspects.

In yet a further aspect, the invention relates to an oligonucleotide comprising, consisting essentially of or consisting of a motif or sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 3);
5′-A[G/A][U/G]C[U/C]C[U/C][C/A]U-3′
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 7)
5′-CGUUUC-3′;
(SEQ ID NO: 8)
5′-CGUGUC-3′;
(SEQ ID NO: 9)
5′-AUGGCCTTTCCGTGCCAAGG-3′;
(SEQ ID NO: 10)
5′-UCCGGCCTCGGAAGCUCUCU-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 19)
5′-CUUUAGTCGTAGTTGCUUCC-3′;
(SEQ ID NO: 20)
5′-UUAAATAATCTAGTTUGAAG-3′;
(SEQ ID NO: 21)
5′-GUGUCCTTCATGCTTUGGAU-3′;
(SEQ ID NO: 22)
5′-AGAAAGAAGCAAAGAUUCAA-3′;
(SEQ ID NO: 23)
5′-AGAUUATCTTCTTTTAAUUU-3′;
(SEQ ID NO: 24)
5′-AAAAGATTATCTTCTUUUAA-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 27)
5′-CUUGUGAAAAGATTAUCUUC-3′;
(SEQ ID NO: 28)
5′-GGUGGCCACAGGCAACGUCA-3′;
(SEQ ID NO: 29)
5′-CCAUGTCCCAGGCCTCCAGU-3′;
(SEQ ID NO: 30)
5′-GCAGUCTCCATGTCCCAGGC-3′;
(SEQ ID NO: 31)
5′-AGCAGTCTCCATGTCCCAGG-3′;
(SEQ ID NO: 32)
5′-AGUGGCACATACCACACCCU-3′;
(SEQ ID NO: 33)
5′-AUUUCCACATGCCCAGUGUU-3′;
(SEQ ID NO: 34)
5′-GGUCCCATCCCTTCTGCUGC-3′;
(SEQ ID NO: 35)
5′-UCUGGTCCCATCCCTUCUGC-3′;
(SEQ ID NO: 36)
5′-GGGUCTCCTCCACACCCUUC-3′;
(SEQ ID NO: 37)
5′-GCAAGGCAGAGAAACUCCAG-3′;
(SEQ ID NO: 38)
5′-GAUGGTTCCAGTCCCUCUUC-3′;
(SEQ ID NO: 39)
5′-UGUUUCCCCGGAGAGCAAUG-3′;
(SEQ ID NO: 40)
5′-AGCCGAACAGAAGGAGCGUC-3′;
(SEQ ID NO: 41)
5′-GCGUAGTTTCTCTTCCUCCC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 46)
5′-GCCGUTTCCATGTCCCAGGC-3′;
(SEQ ID NO: 47)
5′-GCCGUGTCCATGTCCCAGGC-3′;
(SEQ ID NO: 48)
5′-GCGGUATCCATAGTCUCCAU-3′;
(SEQ ID NO: 49)
5′-GCGGUATCCATCAGAUAUCG-3′;
(SEQ ID NO: 50)
5′-CUUUAGTCGTAGTTGUCUCU-3′;
(SEQ ID NO: 51)
5′-UCCGGGTCGTAGTTGCUUCC-3′;
(SEQ ID NO: 52)
5′-UCCGGCCTCGGAGTCUCCAU-3′;
(SEQ ID NO: 53)
5′-UCCGGCCTCGGGAGAUCUCU-3′;
(SEQ ID NO: 54)
5′-GGUATCCCCCCCCCCCCCCC-3′;
(SEQ ID NO: 55)
5′-GGAUUAAAACAGATTAAUAC-3′;
(SEQ ID NO: 56)
5′-GGUAU-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
(SEQ ID NO: 61)
5′-TGTCTG-3′;
(SEQ ID NO: 62)
5′-GTCT-3′;
(SEQ ID NO: 63)
5′-TCTCCG-3′;
(SEQ ID NO: 64)
5′-CTCC-3′;
(SEQ ID NO: 65)
5′-[G/A][A/C]AG[G/C][T/C]T[C/A];
(SEQ ID NO: 66)
5′-AAAGGTTA-3′;
(SEQ ID NO: 67)
5′-GAAGCTTC-3′;
(SEQ ID NO: 68)
5′-GCAGGCTC-3′;
(SEQ ID NO: 69)
5′-A[G/A]GGTT-3′;
(SEQ ID NO: 70)
5′-AGGGTT-3′;
(SEQ ID NO: 71)
5′-AAGGTT-3′;
(SEQ ID NO: 72)
5′-GGTT-3′;
(SEQ ID NO: 73)
5′-[A/G]GCT[T/C][T/C][G/C][T/A]-3′;
(SEQ ID NO: 74)
5′-AGCTTCCT-3′;
(SEQ ID NO: 75)
5′-AGCTTCGA-3′;
(SEQ ID NO: 76)
5′-GGCTTCGT-3′;
(SEQ ID NO: 77)
5′-TGCTTCCT-3′;
(SEQ ID NO: 78)
5′-AGCTCTCT-3′;
(SEQ ID NO: 79)
5′-G[G/C]TT-3′;
(SEQ ID NO: 80)
5′-GCTT-3′;
(SEQ ID NO: 81)
5′-CGGAGGTCTTGGCTTCGTGG-3′;
(SEQ ID NO: 82)
5′-AGGTCTTGGCTTCGTGGAGC-3′;
(SEQ ID NO: 83)
5′-GGGAAAGGTTATGCAAGGTC-3′;
(SEQ ID NO: 84)
5′-CTGTGATCTTGACATGCTGC-3′;
(SEQ ID NO: 85)
5′-ACTGACTGTCTTGAGGGTTC-3′;
(SEQ ID NO: 86)
5′-GCGTGTCTGGAAGCTTCCTT-3′;
(SEQ ID NO: 87)
5′-GAGTCTCTGGAGCTTCCTCT-3′;
(SEQ ID NO: 88)
5′-AGTCGTAGTTGCTTCCTAAC-3′;
(SEQ ID NO: 89)
5′-GTCTTGGCTTCGTGGAGCAG-3′;
(SEQ ID NO: 90)
5′-TTGGCTCGGCTTGCCTACTT-3′;
(SEQ ID NO: 91)
5′-ACAGTGTTGAGATACTCGGG-3′;
(SEQ ID NO: 92)
5′-TCGCACTTCAGTCTGAGCAG-3′;
(SEQ ID NO: 93)
5′-GGTGTCCTTGCACGTGGCTT-3′;
(SEQ ID NO: 94)
5′-TTTGCACACTTCGTACCCAA-3′;
(SEQ ID NO: 95)
5′-GCTGACAAAGATTCACTGGT-3′;
(SEQ ID NO: 96)
5′-GCGGAGGTCTTGGCTTCGTG-3′;
(SEQ ID NO: 97)
5′-CCAAGATCAGCAGTCT-3′;
(SEQ ID NO: 98)
5′-CTTGAAGCATCGTATC-3′;
(SEQ ID NO: 99)
5′-GCACACTTCGTACCCA-3′;
(SEQ ID NO: 100)
5′-GATAGCACCTTCAGCA-3′;
(SEQ ID NO: 101)
5′-CGTATTATAGCCGATT-3′;
(SEQ ID NO: 102)
5′-GCAGGCTCAGTGATGT-3′;
(SEQ ID NO: 103)
5′-GAAAGGTTATGCAAGG-3′;
(SEQ ID NO: 104)
5′-ATGGCCTCCCATCTCC-3′;
(SEQ ID NO: 105)
5′-CGCTTTTCTGTCTGGT-3′;
(SEQ ID NO: 106)
5′-GTGTCTGGAAGCTTCC-3′;
(SEQ ID NO: 107)
5′-TGGCCTCCCATCTCCT-3′;
(SEQ ID NO: 108)
5′-ATCTGGCAGCCCATCA-3′;
(SEQ ID NO: 109)
5′-GAGGTCTTGGCTTCGT-3′;
(SEQ ID NO: 110)
5′-ACACTTCGTGGGGTCC-3′;
(SEQ ID NO: 111)
5′-TTCGTGGGGTCCTTTT-3′;
(SEQ ID NO: 112)
5′-CCACTTGGCAGACCAT-3′;
(SEQ ID NO: 113)
5′-CCATCCATGAGGTCCT-3′;
(SEQ ID NO: 114)
5′-TCCAACACTTCGTGGG-3′;
(SEQ ID NO: 115)
5′-TCTTCATCGGCCCTGC-3′;
(SEQ ID NO: 116)
5′-CCAGCAGGTCAGCAAA-3′;
(SEQ ID NO: 117)
5′-CGCTTTTCTCTCCGGT-3′;
(SEQ ID NO: 118)
5′-GGUAUAGGACTCCAGATGUUUCC-3′;

a variant of the motifs or sequences thereof having at least about 75% sequence identity thereto;
wherein the U may be a T and/or the T may be a U and wherein the oligonucleotide inhibits cGAS activity when administered to a subject or in any assay described herein.

In an embodiment of the above aspect, the oligonucleotide comprises or consists of any sequence in Tables 1, 1A, 2, 2A, 3, 3A, 4, 4A, 5, 5A or 6, with or without the particular modifications defined, that is shown in the Examples to inhibit the activity of cGAS.

In embodiments in which the motif is 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60), wherein the U may be a T, the motif is suitably at or towards the 5′ and/or 3′ end of the oligonucleotide. In certain embodiments, the motif of 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60), wherein the U may be a T, is at or towards the 5′ end of the oligonucleotide.

In one embodiment, the oligonucleotide comprises, consists essentially of or consists of a sequence of 5′-GCGGUATCCATGTCCCAGGC-3′ (SEQ ID NO: 42), 5′-GCGGUATACAGGTCCCAGGC-3′ (SEQ ID NO: 43), 5′-GCUGUTTCCATGTCCCAGGC-3′ (SEQ ID NO: 44), 5′-GCUGUGTCCATGTCCCAGGC-3′ (SEQ ID NO: 45), 5′-GCCGUTTCCATGTCCCAGGC-3′ (SEQ ID NO: 46), 5′-GCCGUGTCCATGTCCCAGGC-3′ (SEQ ID NO: 47), 5′-GCGGUATCCATAGTCUCCAU-3′ (SEQ ID NO: 48), 5′-GCGGUATCCATCAGAUAUCG-3′ (SEQ ID NO: 49), 5′-CUUUAGTCGTAGTTGUCUCU-3′ (SEQ ID NO: 50), 5′-UCCGGGTCGTAGTTGCUUCC-3′ (SEQ ID NO: 51), 5′-UCCGGCCTCGGAGTCUCCAU-3′ (SEQ ID NO: 52), 5′-UCCGGCCTCGGGAGAUCUCU-3′ (SEQ ID NO: 53), 5′-GGUATCCCCCCCCCCCCCCC-3′ (SEQ ID NO: 54) or a variant thereof having at least about 75% sequence identity thereto and wherein the U may be a T and/or the T may be a U.

In one embodiment, the oligonucleotides comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of cGAS.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of cGAS and the oligonucleotide inhibits, or does not substantially inhibit, TLR3, TLR7 and/or TLR9 activity.

In other embodiments, the oligonucleotide comprises, consists essentially of or consists of a sequence of:

5′-mGmCmGmGmUATCCATGTCCmCmAmGmGmC-3′;
5′-mGmCmGmGmUmAmUmCmCmAmUmGmUmCmCmCmAmGmGmC-
3′;
5′-mGmCmGmGmUATACAGGTCCmCmAmGmGmC-3′;
5′-mGmCmUmGmUTTCCATGTCCmCmAmGmGmC-3′;
5′-mGmCmUmGmUGTCCATGTCCmCmAmGmGmC-3′;
5′-mGmCmCmGmUTTCCATGTCCmCmAmGmGmC-3′;
5′-mGmCmCmGmUGTCCATGTCCmCmAmGmGmC-3′;
5′-mGmCmGmGmUATCCATAGTCmUmCmCmAmU-3′;
5′-mGmCmGmGmUATCCATCAGAmUmAmUmCmG-3′;
5′-mCmUmUmUmAGTCGTAGTTGmUmCmUmCmU-3′;
5′-mUmCmCmGmGGTCGTAGTTGmCmUmUmCmC-3′;
5′-mUmCmCmGmGCCTCGGAGTCmUmCmCmAmU-3′,
5′-mUmCmCmGmGCCTCGGGAGAmUmCmUmCmU-3′;
5′-mGmGmUATCCCCCCCCCCCCCCC-3′;
5′-mGmGmUmAmU-3′;
5′-mGmGmUmA-3′;
5′-mGmUmAmU-3′;
5′-mGmGmU-3′;
5′-mGmUmA-3′;

or a variant thereof having at least about 75% sequence identity thereto and wherein the U may be a T and/or the T may be a U and wherein m is a modified base and/or has a modified backbone.

In another embodiment, the oligonucleotide comprises, consists essentially of or consists of a sequence of 5′-GCGGUATCCATGTCCCAGGC-3′ (SEQ ID NO: 42), or a variant thereof having at least about 75% sequence identity thereto and wherein the U may be a T and/or the T may be a U.

In some embodiments, the oligonucleotide comprises, consists essentially of or consists of a sequence of 5′-GGUAU-3′ (SEQ ID NO: 56) or a variant thereof having at least about 75% sequence identity thereto and wherein the U may be a T.

In other embodiments, the oligonucleotide comprises, consists essentially of or consists of a sequence of 5′-GGUA-3′ (SEQ ID NO: 57) or a variant thereof having at least about 75% sequence identity thereto and wherein the U may be a T.

In some embodiments, the oligonucleotide comprises, consists essentially of or consists of a sequence of 5′-GUAU-3′ (SEQ ID NO: 58) or a variant thereof having at least about 75% sequence identity thereto and wherein the U may be a T.

In certain embodiments, the oligonucleotide comprises, consists essentially of or consists of a sequence of 5′-GGU-3′ (SEQ ID NO: 59) or a variant thereof having at least about 75% sequence identity thereto and wherein the U may be a T.

In some embodiments, the oligonucleotide comprises, consists essentially of or consists of a sequence of 5′-GUA-3′ (SEQ ID NO: 60) or a variant thereof having at least about 75% sequence identity thereto and wherein the U may be a T.

In particular embodiments, the oligonucleotide comprises, consists essentially of or consists of a sequence of 5′-mGmCmGmGmUATCCATGTCCmCmAmGmGmC-3′ or a variant thereof having at least about 75% sequence identity thereto and wherein the U may be a T and/or the T may be a U and wherein m is a modified base and/or has a modified backbone.

In particular embodiments, the oligonucleotide comprises, consists essentially of or consists of a sequence of 5′-mGmCmGmGmUmAmUmCmCmAmUmGmUmCmCmCmAmGmGmC-3′ or a variant thereof having at least about 75% sequence identity thereto and wherein the U may be a T and/or the T may be a U and wherein m is a modified base and/or has a modified backbone.

In any embodiment of this aspect, the oligonucleotide that inhibits cGAS activity is at least 15, 16, 17, 18, 19 or 20 nucleotides in length.

In another aspect, the invention provides an oligonucleotide comprising a motif or sequence selected from the group consisting of:

(SEQ ID NO: 140)
5′[C/U]CUUCU-3′;
(SEQ ID NO: 141)
5′-CACCCTTCTCTCTGGUCCCA-3′;
(SEQ ID NO: 142)
5′-CCUUCTCTCTGGTCCCAUCC-3′;
(SEQ ID NO: 143)
5′-UCUCUGGTCCCATCCCUUCU-3′;
(SEQ ID NO: 144)
5′-AUAUCTGCTGCCCACCUUCU-3′;
(SEQ ID NO: 145)
5′-GUCCCATCCCTTCTGCUGCC-3′;
(SEQ ID NO: 146)
5′-GUCUCCTCCACACCCUUCUC-3′;
(SEQ ID NO: 147)
5′-CAGGCCTCCAGTGTCUUCUC-3′;
(SEQ ID NO: 148)
5′-UCCCAACTCTTCTAACUCGU-3′;

a variant of the motifs or sequences thereof having at least about 75% sequence identity thereto;
wherein the U may be a T and/or the T may be a U and wherein the oligonucleotide does not inhibit or exhibits reduced inhibition of cyclic-GMP-AMP synthase (cGAS) activity when administered to a subject or in any assay described herein.

In still another aspect, the invention relates to an oligonucleotide comprising, consisting essentially of or consisting of a motif or sequence selected from the group consisting of:

(SEQ ID NO: 152)
5′-G[G/C]CCT[C/G]-3′;
(SEQ ID NO: 153)
5′-CUU-3′, wherein the motif is within 10 bases
of the 5′ and/or 3′ end of the oligonucleotide;
(SEQ ID NO: 27)
5′-CUUGUGAAAAGATTAUCUUC-3′;
(SEQ ID NO: 154)
5′-CUUCUCTCTGGTCCCAUCCC-3′;
(SEQ ID NO: 142)
5′-CCUUCTCTCTGGTCCCAUCC-3′;
(SEQ ID NO: 155)
5′-CCCUUCTCTCTGGTCCCAUC-3′;
(SEQ ID NO: 156)
5′-ACCCUTCTCTCTGGTCCCAU-3′;
(SEQ ID NO: 157)
5′-CUUCCACAATCAAGACAUUC-3′;
(SEQ ID NO: 158)
5′-CUUCGTGGGGTCCTTUUCAC-3′;
(SEQ ID NO: 159)
5′-CACUUCGTGGGGTCCUUUUC-3′;
(SEQ ID NO: 160)
5′-CCAACACTTCGTGGGGUCCU-3′;
(SEQ ID NO: 10)
5′-UCCGGCCTCGGAAGCUCUCU-3′;
(SEQ ID NO: 29)
5′-CCAUGTCCCAGGCCTCCAGU-3′;
(SEQ ID NO: 161)
5′-UUGGCCTGTGGATGCUUUGU-3′;
(SEQ ID NO: 162)
5′-AAAUGTCCTGGCCCTCACUG-3′;
(SEQ ID NO: 52)
5′-UCCGGCCTCGGAGTCUCCAU-3′;
(SEQ ID NO: 163)
5′-UCCGGCCTCGGCAGAUAUCG-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 16)
5′-CAUUAGGTGCAGAAAUCUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 51)
5′-UCCGGGTCGTAGTTGCUUCC-3′;
(SEQ ID NO: 164)
5′-CCUAGAAAGAAGCAAAGAUU-3′;
(SEQ ID NO: 165)
5′-GAUUAAAACAGATTAAUACA-3′;
(SEQ ID NO: 55)
5′-GGAUUAAAACAGATTAAUAC-3′;
(SEQ ID NO: 166)
5′-AAUUUAAAGCATGAAUAUUA-3′;
(SEQ ID NO: 41)
5′-GCGUAGTTTCTCTTCCUCCC-3′;
(SEQ ID NO: 167)
5′-UGACAAAACAATAATAACAG-3′;
(SEQ ID NO: 168)
5′-ACA-3′, wherein the motif is at or towards
the 5′ end of the oligonucleotide;
(SEQ ID NO: 169)
5′-CAC-3′, wherein the motif is at or towards
the 5′ end of the oligonucleotide;
(SEQ ID NO: 170)
5′-ACACTTCGTGGGGTCCTTTT-3′;
(SEQ ID NO: 86)
5′-GCGTGTCTGGAAGCTTCCTT-3′;
(SEQ ID NO: 171)
5′-TCAAAGGACTGAGGAAAGGG-3′;
(SEQ ID NO: 172)
5′-ATCCAACACTTCGTGGGGTC-3′;
(SEQ ID NO: 173)
5′-GCCCATCCATGAGGTCCTGG-3′;
(SEQ ID NO: 174)
5′-GGGTATCGAAAGAGTCTGGA-3′;
(SEQ ID NO: 175)
5′-GGTTTTGGCTGGGATCAAGT-3′;
(SEQ ID NO: 176)
5′-GCGACTATACGCGCAATATG-3′;
(SEQ ID NO: 85)
5′-ACTGACTGTCTTGAGGGTTC-3′;
(SEQ ID NO: 177)
5′-AACACTTCGTGGGGTCCTTT-3′;
(SEQ ID NO: 178)
5′-GTCCAAGATCAGCAGTCTCA-3′;
(SEQ ID NO: 91)
5′-ACAGTGTTGAGATACTCGGG-3′;
(SEQ ID NO: 179)
5′-TGGGCTGGAATCCGAGTTAT-3′;
(SEQ ID NO: 180)
5′-CGGCATCCACCACGTCGTCC-3′;
(SEQ ID NO: 181)
5′-GCGTATTATAGCCGATTAAC-3′;
(SEQ ID NO: 182)
5′-GGAGGTCTTGGCTTCGTGGA-3′;
(SEQ ID NO: 183)
5′-TGGGTTACGGCTCAGTATGG-3′;
(SEQ ID NO: 184)
5′-CCGCCATGTTTCTTCTTGGA-3′;
(SEQ ID NO: 185)
5′-AGCTTCGAGGCCCCAG-3′;
(SEQ ID NO: 186)
5′-GCCATGTTTCTTCTTG-3′;
(SEQ ID NO: 187)
5′-CACTTCGTGGGGTCCT-3′;
(SEQ ID NO: 188)
5′-CGGCCTCGGAAGCTCT-3′;
(SEQ ID NO: 110)
5′-ACACTTCGTGGGGTCC-3′;
(SEQ ID NO: 189)
5′-TGCACACTTCGTACCC-3′;
(SEQ ID NO: 190)
5′-CCACATCCTGTGGCTC-3′;
(SEQ ID NO: 191)
5′-CTGCAGCTTCCTTGTC-3′;
(SEQ ID NO: 192)
5′-ACTTCGTGGGGTCCTT-3′;
(SEQ ID NO: 193)
5′-CCCACTTGGCAGACCA-3′;
(SEQ ID NO: 194)
5′-GTCCCCTGTTGACTGG-3′;
(SEQ ID NO: 195)
5′-ACGTTCAGTCCTGTCC-3′;
(SEQ ID NO: 196)
5′-GGTCATTACAATAGCT-3′;
(SEQ ID NO: 197)
5′-TCGTGGGGTCCTTTTC-3′;
(SEQ ID NO: 106)
5′-GTGTCTGGAAGCTTCC-3′;
(SEQ ID NO: 104)
5′-ATGGCCTCCCATCTCC-3′;
(SEQ ID NO: 198)
5′-TGCTCCTCGGTCTCCC-3′;
(SEQ ID NO: 199)
5′-GCATCCACCACGTCGT-3′;
(SEQ ID NO: 200)
5′-CTTCGTGGGGTCCTTT-3′;
(SEQ ID NO: 201)
5′-AGGCCCTTCGCACTTC-3′;
5′-GCGGUATCCATGTCCCAGGC-3′;
5′-GCUGUTTCCATGTCCCAGGC-3′;
5′-GCUGUGTCCATGTCCCAGGC-3′
5′-GCCGUTTCCATGTCCCAGGC-3′;
5′-GCGGUATCC-3′;
5′-GCUGUTTCC-3′;
5′-GCUGUGTCC-3′;
5′-GCCGUTTCC-3′;
5′-CUUCGTGGGGTCCTTUUCAC-3′;
5′-CUUCGTGGG-3′;
5′-UCG-3′;
5′-ACG-3′;
5′-ACC-3′;
5′-CGC-3′;
5′-GAU-3′;
5′-GGG-3′;
5′-AGC-3′;
5′-UUC-3′;
5′-UUG-3′;
5′-CAC-3′;

and
a variant of the motifs or sequences thereof having at least about 75% sequence identity thereto;
wherein the U may be a T and/or the T may be a U and wherein the oligonucleotide inhibits TLR9 activity when administered to a subject or in any assay described herein.

In one embodiment, one, two or more bases of the oligonucleotide are modified bases or one, two or more internucletide linkages have a modified backbone, preferably wherein the modified base is 2′OMe.

In one embodiment, the oligonucleotide comprises, consists essentially of or consists of a sequence of 5′-UCCGGCCTCGGAGTCUCCAU-3′ (SEQ ID NO: 52), 5′-GCGGUATCCATAGTCUCCAU-3′ (SEQ ID NO: 48), 5′-CCAACACTTCGTGGGGUCCU-3′ (SEQ ID NO: 160), 5′-CACUUCGTGGGGTCCUUUUC-3′ (SEQ ID NO: 159), 5′-UGACAAAACAATAATAACAG-3′ (SEQ ID NO: 167) or a variant thereof having at least about 75% sequence identity thereto and wherein the U may be a T and/or the T may be a U.

In one embodiment, the oligonucleotide comprises, consists essentially of or consists of a sequence of 5′-ACC-3′, 5′-CGC-3′, 5′-GAU-3′, 5′-GGG-3′, 5′-UCG-3′ or 5′-ACG-3′, preferably 5′-mAmCmC-3′, 5′-mCmGmC-3′, 5′-mGmAmU-3′, 5′-mGmGmG-3′, 5′-mUmCmG-3′ or 5′mAmCmG-3′, wherein m is a modified base and/or has a modified backbone, preferably wherein the modified base is 2′OMe.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR9.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR9 and the oligonucleotide inhibits, or does not substantially inhibit, TLR3, TLR7 and/or cGAS activity.

In one embodiment, the oligonucleotide inhibits TLR9 activity and potentiates TLR8 activity, for example an oligonucleotide comprising, consisting essentially of or consisting of a sequence of 5′-UCG-3′ or 5′-CGC-3′, preferably 5′-mUmCmG-3′ or 5′-mCmGmC-3′, wherein m is a modified base and/or has a modified backbone, preferably wherein the modified base is 2′OMe. In other embodiments, the oligonucleotide inhibits TLR9 activity and does not substantially potentiate TLR8 activity or inhibits TLR8 activity, for example an oligonucleotide comprising, consisting essentially of or consisting of a sequence of 5′-GAU-3′, preferably 5′-mGmAmU-3′, wherein m is a modified base and/or has a modified backbone, preferably wherein the modified base is 2′OMe.

In any embodiment, the oligonucleotide that inhibits TLR9 activity is at least 3, 4, 5, 6, 7, 8, 9, 10, 11, 12, 13, 14, 15, 16, 17, 18, 19, 20, 30, 40 or 50 nucleotides in length. In one embodiment, the oligonucleotide that inhibits TLR9 activity is at least 3 but less than or equal to 20 nucleotides in length, optionally at least 9 but less than or equal to 20 nucleotides in length.

In other embodiments, the oligonucleotide comprises, consists essentially of or consists of a sequence of:

5′-mUmCmCmGmGCCTCGGAGTCmUmCmCmAmU-3′;
5′-mGmCmGmGmUATCCATAGTCmUmCmCmAmU-3′;
5′-mCmCmAmAmCACTTCGTGGGmGmUmCmCmU-3′;
5′-mCmAmCmUmUCGTGGGGTCCmUmUmUmUmC-3′;
5′-mUmGmAmCmAAAACAATAATmAmAmCmAmG-3′;
5′-mUmCmCmGmGCCTCGGAAGCmUmCmUmCmU-3′;
5′-mUmCmCmGmGCCTCGGAGTCmUmCmCmAmU-3′;
5′-mUmCmCmGmGCCTCGGCAGAmUmAmUmCmG-3′;

or a variant thereof having at least about 75% sequence identity thereto and wherein the U may be a T and/or the T may be a U and wherein m is a modified base and/or has a modified backbone.

In other embodiments, the oligonucleotide comprises, consists essentially of or consists of a sequence of:

5′-mGmCmGmGmUATCCATGTCCmCmAmGmGmC-3′;
5′-mGmCmUmGmUTTCCATGTCCmCmAmGmGmC-3′;
5′-mGmCmUmGmUGTCCATGTCCmCmAmGmGmC-3′;
5′-mGmCmCmGmUTTCCATGTCCmCmAmGmGmC-3′;
5′-mGmCmGmGmUATCC-3′;
5′-mGmCmUmGmUTTCC-3′;
5′-mGmCmUmGmUGTCC-3′;
5′-MGmCmCmGmUTTCC-3′;
5′-mCmUmUmCmGTGGGGTCCTTmUmUmCmAmC-3′;
and
5′-mCmUmUmCmGTGGG-3′;

or a variant thereof having at least about 75% sequence identity thereto and wherein the T may be a U and wherein m is a modified base and/or has a modified backbone. Preferably, each base has a modified backbone, and the modified backbone is phosphorothioate.

In other embodiments, the oligonucleotide comprises, consists essentially of or consists of a sequence of:

5′-mG*mC*mG*mG*mU*A*T*C*C*A*T*G*T*C*C*mC*mA*mG*
mG*mC-3′;
5′-mU*mC*mC*mG*mG*C*C*T*C*G*G*A*A*G*C*mU*mC*mU*
mC*mU-3′;
5′-mU*mC*mC*mG*mG*C*C*T*C*G*G*A*G*T*C*mU*mC*mC*
mA*mU-3′;
5′-mU*mC*mC*mG*mG*C*C*T*C*G*G*C*A*G*A*mU*mA*mU*
mC*mG-3′;
5′-mG*mC*mU*mG*mU*T*T*C*C*A*T*G*T*C*C*mC*mA*mG*
mG*mC-3′;
5′-mG*mC*mU*mG*mU*G*T*C*C*A*T*G*T*C*C*mC*mA*mG*
mG*mC-3′;
5′-mG*mC*mC*mG*mU*T*T*C*C*A*T*G*T*C*C*mC*mA*mG
*mG*mC-3′;
5′-mG*mC*mG*mG*mU*A*T*C*C*-3′;
5′-mG*mC*mU*mG*mU*T*T*C*C*-3′;
5′-mG*mC*mU*mG*mU*G*T*C*C*-3′;
5′-mG*mC*mC*mG*mU*T*T*C*C*-3′;
5′-mC*mU*mU*mC*mG*T*G*G*G*G*T*C*C*T*T*mU*mU*mC*
mA*mC;
and
5′-mC*mU*mU* mC*mG*T*G*G*G*-3′;

wherein the U may be a T and/or the T may be a U and wherein ‘m’ indicates 2′OMe base, and * denotes the phosphorothioate backbone.

In another aspect, the invention resides in an oligonucleotide which comprises:
a) a 5′ region comprising bases which are modified and/or which have a modified backbone,
   b) a middle region comprising ribonucleic acid, deoxyribonucleic acid, or combination thereof, bases, which optionally have a modified backbone, wherein at least about 50% of the bases of the middle region are adenine bases; and
   c) a 3′ region comprising bases which are modified and/or which have a modified backbone;
wherein the oligonucleotide inhibits TLR9 activity when administered to a subject.

In an embodiment, the oligonucleotide comprises a motif or sequence selected from the group consisting of:

(SEQ ID NO: 165)
5′-GAUUAAAACAGATTAAUACA-3′;
(SEQ ID NO: 55)
5′-GGAUUAAAACAGATTAAUAC-3′;
(SEQ ID NO: 27)
5′-CUUGUGAAAAGATTAUCUUC-3′;
(SEQ ID NO: 164)
5′-CCUAGAAAGAAGCAAAGAUU-3′;
(SEQ ID NO: 166)
5′-AAUUUAAAGCATGAAUAUUA-3′;

and
a variant of the motifs or sequences thereof having at least about 75% sequence identity thereto;
wherein the U may be a T and/or the T may be a U.

In an embodiment, the oligonucleotide comprises a motif or sequence selected from the group consisting of:

(SEQ ID NO: 165)
5′-GAUUAAAACAGATTAAUACA-3′;
(SEQ ID NO: 55)
5′-GGAUUAAAACAGATTAAUAC-3′;
(SEQ ID NO: 27)
5′-CUUGUGAAAAGATTAUCUUC-3′;
(SEQ ID NO: 164)
5′-CCUAGAAAGAAGCAAAGAUU-3′;
(SEQ ID NO: 166)
5′-AAUUUAAAGCATGAAUAUUA-3′;

and
a variant of the motifs or sequences thereof having at least about 75% sequence identity thereto;
wherein the U may be a T and/or the T may be a U.

In one further aspect, the invention provides an oligonucleotide comprising, consisting essentially of or consisting of a motif or sequence selected from the group consisting of:

(SEQ ID NO: 1)
5′-[A/G]GU[A/C][U/C]C-3′;
(SEQ ID NO: 2)
5′-A[G/A][U/G]C[U/C]C-3′;
(SEQ ID NO: 212)
5′-A[G/A][U/G]C[U/C]C[U/C][C/A]U-3′;
(SEQ ID NO: 4)
5′-GGUAUA-3′;
(SEQ ID NO: 5)
5′-UGUUUC-3′;
(SEQ ID NO: 6)
5′-UGUGUC-3′;
(SEQ ID NO: 8)
5′-CGUGUC-3′;
(SEQ ID NO: 30)
5′-GCAGUCTCCATGTCCCAGGC-3′;
(SEQ ID NO: 38)
5′-GAUGGTTCCAGTCCCUCUUC-3′;
(SEQ ID NO: 31)
5′-AGCAGTCTCCATGTCCCAGG-3′;
(SEQ ID NO: 36)
5′-GGGUCTCCTCCACACCCUUC-3′;
(SEQ ID NO: 28)
5′-GGUGGCCACAGGCAACGUCA-3′;
(SEQ ID NO: 46)
5′-GCCGUTTCCATGTCCCAGGC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 47)
5′-GCCGUGTCCATGTCCCAGGC-3′;
(SEQ ID NO: 151)
5′-GCGGUAUCCAUGUCCCAGGC-3′;
(SEQ ID NO: 54)
5′-GGUATCCCCCCCCCCCCCCC-3′;
(SEQ ID NO: 145)
5′-GUCCCATCCCTTCTGCUGCC-3′;
(SEQ ID NO: 213)
5′-UUCUCTCTGGTCCCAUCCCU-3′;
(SEQ ID NO: 214)
5′-GUUCAGTCAGATCGCUGGGA-3′;
(SEQ ID NO: 215)
5′-AUGACATTTCGTGGCUCCUA-3′;
(SEQ ID NO: 216)
5′-UCUCCATGTCCCAGGCCUCC-3′;
(SEQ ID NO: 217)
5′-AGUCUCCATGTCCCAGGCCU-3′;
(SEQ ID NO: 29)
5′-CCAUGTCCCAGGCCTCCAGU-3′;
(SEQ ID NO: 37)
5′-GCAAGGCAGAGAAACUCCAG-3′;
(SEQ ID NO: 55)
5′-GGAUUAAAACAGATTAAUAC-3′;
(SEQ ID NO: 40)
5′-AGCCGAACAGAAGGAGCGUC-3′;
(SEQ ID NO: 10)
5′-UCCGGCCTCGGAAGCUCUCU-3′;
(SEQ ID NO: 52)
5′-UCCGGCCTCGGAGTCUCCAU-3′;
(SEQ ID NO: 48)
5′-GCGGUATCCATAGTCUCCAU-3′;
(SEQ ID NO: 165)
5′-GAUUAAAACAGATTAAUACA-3′;
(SEQ ID NO: 167)
5′-UGACAAAACAATAATAACAG-3′;
(SEQ ID NO: 160)
5′-CCAACACTTCGTGGGGUCCU-3′;
(SEQ ID NO: 21)
5′-GUGUCCTTCATGCTTUGGAU-3′;
(SEQ ID NO: 218)
5′-GUCCCAGGCCTCCAGUGUCU-3′;
(SEQ ID NO: 161)
5′-UUGGCCTGTGGATGCUUUGU-3′;
(SEQ ID NO: 219)
5′-GUCCGTACCTCCACCCACCG-3′;
(SEQ ID NO: 220)
5′-GUGUUTTTAATTTTGUAGAG-3′;
(SEQ ID NO: 221)
5′-GUCAAACCTAGAAAGAAGCA-3′;
(SEQ ID NO: 222)
5′-GGUCUCCTCCACACCCUUCU-3′;
(SEQ ID NO: 146)
5′-GUCUCCTCCACACCCUUCUC-3′;
(SEQ ID NO: 223)
5′-UGAUGATGCTTGCAGGAGGC-3′;
(SEQ ID NO: 20)
5′-UUAAATAATCTAGTTUGAAG-3′;
(SEQ ID NO: 224)
5′-AAAGCAGTCTCCATGUCCCA-3′;
(SEQ ID NO: 41)
5′-GCGUAGTTTCTCTTCCUCCC-3′;
(SEQ ID NO: 35)
5′-UCUGGTCCCATCCCTUCUGC-3′;
(SEQ ID NO: 225)
5′-UAUUUCCACATGCCCAGUGU-3′;
(SEQ ID NO: 39)
5′-UGUUUCCCCGGAGAGCAAUG-3′;
(SEQ ID NO: 226)
5′-UUAGCTCCTTGCCTCGUUCC-3′;
(SEQ ID NO: 33)
5′-AUUUCCACATGCCCAGUGUU-3′;
(SEQ ID NO: 227)
5′-UGGCGTAGTTTCTCTUCCUC-3′;
(SEQ ID NO: 228)
5′-UGACATTTCGTGGCTCCUAC-3′;
(SEQ ID NO: 25)
5′-GAAAAGATTATCTTCUUUUA-3′;
(SEQ ID NO: 26)
5′-UGUGAAAAGATTATCUUCUU-3′;
(SEQ ID NO: 229)
5′-UUGUGAAAAGATTATCUUCU-3′;
(SEQ ID NO: 230)
5′-UUUGAAATTCAGAAGAUUUG-3′;
(SEQ ID NO: 231)
5′-AAGCAGTCTCCATGTCCCAG-3′;
(SEQ ID NO: 232)
5′-AGGAUTAAAACAGATUAAUA-3′;
(SEQ ID NO: 23)
5′-AGAUUATCTTCTTTTAAUUU-3′;
(SEQ ID NO: 148)
5′-UCCCAACTCTTCTAACUCGU-3′;
(SEQ ID NO: 233)
5′-UAAAATAAGGGGAATAGGGG-3′;
(SEQ ID NO: 32)
5′-AGUGGCACATACCACACCCU-3′;
(SEQ ID NO: 234)
5′-AAGAUTATCTTCTTTUAAUU-3′;
(SEQ ID NO: 22)
5′-AGAAAGAAGCAAAGAUUCAA-3′;
(SEQ ID NO: 235)
5′-UCCCATCCCTTCTGCUGCCA-3′;
(SEQ ID NO: 156)
5′-ACCCUTCTCTCTGGTCCCAU-3′;
(SEQ ID NO: 236)
5′-AAUAUCTGCTGCCCACCUUC-3′;
(SEQ ID NO: 237)
5′-UCUCUCTGGTCCCATCCCUU-3′;
(SEQ ID NO: 238)
5′-AGGCCTCCAGTGTCTUCUCC-3′;
(SEQ ID NO: 239)
5′-CAAGCCCCAGCGTTCCUCCG-3′;
(SEQ ID NO: 162)
5′-AAAUGTCCTGGCCCTCACUG-3′;
(SEQ ID NO: 166)
5′-AAUUUAAAGCATGAAUAUUA-3′;
(SEQ ID NO: 24)
5′-AAAAGATTATCTTCTUUUAA-3′;
(SEQ ID NO: 144)
5′-AUAUCTGCTGCCCACCUUCU-3′;
(SEQ ID NO: 240)
5′-CAGUCTCCATGTCCCAGGCC-3′;
(SEQ ID NO: 241)
5′-AAAGATTATCTTCTTUUAAU-3′;
(SEQ ID NO: 155)
5′-CCCUUCTCTCTGGTCCCAUC-3′;
(SEQ ID NO: 9)
5′-AUGGCCTTTCCGTGCCAAGG-3′;
(SEQ ID NO: 11)
5′-GCAUUCCGTGCGGAAGCCUU-3′;
(SEQ ID NO: 12)
5′-GGCCGAACTTTCCCGCCUUA-3′;
(SEQ ID NO: 13)
5′-GGUCUTGGCTTCGTGGAGCA-3′;
(SEQ ID NO: 14)
5′-GGAGCTTCGAGGCCCCAGGC-3′;
(SEQ ID NO: 15)
5′-GGUGGTCCACAACCCCUUUC-3′;
(SEQ ID NO: 17)
5′-UUCUGGGGACTTCCAGUUUA-3′;
(SEQ ID NO: 18)
5′-UGAUUCCAAAGCCAGGGUUA-3′;
(SEQ ID NO: 242)
5′-GGGUATCGAAAGAGTCUGGA-3′;
(SEQ ID NO: 243)
5′-CUUGCACGTGGCTTCGUCUC-3′;
(SEQ ID NO: 244)
5′-GUGUCCTTGCACGTGGCUUC-3′;
(SEQ ID NO: 245)
5′-GUAAAAAGCTTTTGAAGUGA-3′;
(SEQ ID NO: 246)
5′-AUGCCATCCACTTGAUAGGC-3′;
(SEQ ID NO: 247)
5′-UGAAGTAAAAATCAAUAGCG-3′;
(SEQ ID NO: 248)
5′-AAGGCCCTTCGCACTUCUUA-3′;
(SEQ ID NO: 249)
5′-GUACUCGTCGGCATCCACCA-3′;
(SEQ ID NO: 250)
5′-GUCCUTGCACGTGGCUUCGU-3′;
(SEQ ID NO: 251)
5′-GCCCATCCATGAGGTCCUGG-3′;
(SEQ ID NO: 252)
5′-GUAAAAGGAGAAAACUAUCU-3′;
(SEQ ID NO: 253)
5′-UUGAAGTGAAGTAAAAGGAG-3′;
(SEQ ID NO: 254)
5′-GUUACTCGTGCCTTGGCAAA-3′;
(SEQ ID NO: 255)
5′-GUCCAAGATCAGCAGUCUCA-3′;
(SEQ ID NO: 256)
5′-UUCAATGGGAGAATAAAGCA-3′;
(SEQ ID NO: 257)
5′-GCAAGGCCCTTCGCACUUCU-3′;
(SEQ ID NO: 258)
5′-GGGUCCACCACTAGCCAGUA-3′;
(SEQ ID NO: 259)
5′-GUAGAGAAATTATTTUAGGA-3′;
(SEQ ID NO: 260)
5′-AUCCACCACGTCGTCCAUGU-3′;
(SEQ ID NO: 261)
5′-GGCAUCCACCACGTCGUCCA-3′;
(SEQ ID NO: 262)
5′-UUACUTTAAAAGCAAAAGGA-3′;
(SEQ ID NO: 263)
5′-UUUGAAGTGAAGTAAAAGGA-3′;
(SEQ ID NO: 264)
5′-GAAGUGAAGTAAAAGGAGAA-3′;
(SEQ ID NO: 265)
5′-GGCCATCTCTGCTTCUUGGU-3′;
(SEQ ID NO: 266)
5′-UGGGCTGGAATCCGAGUUAU-3′;
(SEQ ID NO: 267)
5′-GGAGATTTCAGAGCAGCUUC-3′;
(SEQ ID NO: 268)
5′-UUUACGGTTTTCAGAAUAUC-3′;
(SEQ ID NO: 269)
5′-GCGUGTCTGGAAGCTUCCUU-3′;
(SEQ ID NO: 270)
5′-GCUUATTTTAAGCATAUUAA-3′;
(SEQ ID NO: 271)
5′-UUAUUTTAAGCATATUAAAA-3′;
(SEQ ID NO: 272)
5′-UUCUGCAGCTTCCTTGUCCU-3′;
(SEQ ID NO: 273)
5′-AUUACTTTAAAAGCAAAAGG-3′;
(SEQ ID NO: 274)
5′-AUUUUAAGCATATTAAAAAG-3′;
(SEQ ID NO: 275)
5′-UGUGGCTTGTCCTCAGACAU-3′;
(SEQ ID NO: 276)
5′-AAAAGGAGAAAACTAUCUUC-3′;
(SEQ ID NO: 277)
5′-GGGUCCATACCCAAGGCAUC-3′;
(SEQ ID NO: 278)
5′-ACAGUGTTGAGATACUCGGG-3′;
(SEQ ID NO: 279)
5′-GGAUCTGCATGCCCTCAUCU-3′;
(SEQ ID NO: 280)
5′-AGUAAAAAGCTTTTGAAGUG-3′;
(SEQ ID NO: 281)
5′-GUCGUGGCAAATAGTCCUAG-3′;
(SEQ ID NO: 282)
5′-GGAGATCAGATGAGAGGAGC-3′;
(SEQ ID NO: 283)
5′-GUGGUTAAGTACATGAGCUC-3′;
(SEQ ID NO: 284)
5′-GGACACTTAGCTGTTCCUCG-3′;
(SEQ ID NO: 285)
5′-GUCUCTACTGTTACCUCUGA-3′;
(SEQ ID NO: 286)
5′-GAGUUCTTCGTAGGCUUCUG-3′;
(SEQ ID NO: 287)
5′-AAAGUCAAAAAGAAAAACUG-3′;
(SEQ ID NO: 288)
5′-AAAAGTGGGAAATAAAGGUU-3′;
(SEQ ID NO: 289)
5′-AGUUUATAGATTTCAAGUAG-3′;
(SEQ ID NO: 290)
5′-AAAAAGTGGGAAATAAAGGU-3′;
(SEQ ID NO: 291)
5′-UUUAUATTACAAAGCUACUU-3′;
(SEQ ID NO: 292)
5′-UGCUATTCATATTTTUAUUU-3′;
(SEQ ID NO: 56)
5′-GGUAU-3′;
(SEQ ID NO: 293)
5′-[G/A/C][G/A][G/A/C][T/A/C]TC-3′;
(SEQ ID NO: 294)
5′-[G/A/C]G[G/A/C][T/A/C]TC-3′;
(SEQ ID NO: 295)
5′-GGCTTC-3′;
(SEQ ID NO: 296)
5′-GGCATC-3′;
(SEQ ID NO: 297)
5′-AGCTTC-3′;
(SEQ ID NO: 298)
5′-GGAATC-3′;
(SEQ ID NO: 299)
5′-CACATC-3′;
(SEQ ID NO: 204)
5′-GGCCTC-3′;
(SEQ ID NO: 300)
5′-CACTTC-3′;
(SEQ ID NO: 301)
5′-AAGATC-3′;
(SEQ ID NO: 302)
5′-TGTCCTTGCACGTGGCTTCG-3′;
(SEQ ID NO: 94)
5′-TTTGCACACTTCGTACCCAA-3′;
(SEQ ID NO: 303)
5′-GTCCACATCCTGTGGCTCGT-3′;
(SEQ ID NO: 304)
5′-TGTGATGGCCTCCCATCTCC-3′;
(SEQ ID NO: 175)
5′-GGTTTTGGCTGGGATCAAGT-3′;
(SEQ ID NO: 93)
5′-GGTGTCCTTGCACGTGGCTT-3′;
(SEQ ID NO: 305)
5′-GGTCCATACCCAAGGCATCC-3′;
(SEQ ID NO: 306)
5′-GTGTCTTCATCGGCCCTGCC-3′;
(SEQ ID NO: 89)
5′-GTCTTGGCTTCGTGGAGCAG-3′;
(SEQ ID NO: 95)
5′-GCTGACAAAGATTCACTGGT-3′;
(SEQ ID NO: 103)
5′-GAAAGGTTATGCAAGG-3′;
(SEQ ID NO: 307)
5′-GACTATACGCGCAATA-3′;
(SEQ ID NO: 308)
5′-TGTGATGGCCTCCCAT-3′;
(SEQ ID NO: 114)
5′-TCCAACACTTCGTGGG-3′;
(SEQ ID NO: 106)
5′-GTGTCTGGAAGCTTCC-3′;
(SEQ ID NO: 98)
5′-CTTGAAGCATCGTATC-3′;
(SEQ ID NO: 309)
5′-TCGTAGTTGCTTCCTA-3′;
(SEQ ID NO: 105)
5′-CGCTTTTCTGTCTGGT-3′;
(SEQ ID NO: 310)
5′-GGCTGGAATCCGAGTT-3′;
(SEQ ID NO: 100)
5′-GATAGCACCTTCAGCA-3′;
(SEQ ID NO: 311)
5′-AGGACTCCAGATGTTT-3′;
(SEQ ID NO: 312)
5′-GTGATCTTGACATGCT-3′;
(SEQ ID NO: 313)
5′-AGATTTCAGAGCAGCT-3′;
(SEQ ID NO: 314)
5′-GGTTACGGCTCAGTAT-3′;
(SEQ ID NO: 315)
5′-GTTCAGTCCTGTCCAT-3′;
(SEQ ID NO: 316)
5′-AGGTCTTGGCTTCGTG-3′;
(SEQ ID NO: 191)
5′-CTGCAGCTTCCTTGTC-3′;
(SEQ ID NO: 317)
5′-GTCCTTGCACGTGGCT-3′;
(SEQ ID NO: 318)
5′-GTCTCTGGAGCTTCCT-3′;
(SEQ ID NO: 319)
5′-GGTCTTGGCTTCGTGG-3′;
(SEQ ID NO: 57)
5′-GGUA-3′;
(SEQ ID NO: 58)
5′-GUAU-3′;
(SEQ ID NO: 59)
5′-GGU-3′;
(SEQ ID NO: 60)
5′-GUA-3′;
5′-GUC-3′;
5′-GUG-3′;
5′-GUU-3′;
5′-GGC-3′;
5′-AUC-3′;
5′-GAG-3′;
5′-GGA-3′;
5′-TTT-3′;
5′-TCT-3′;
5′-GAA-3′;
5′-GAC-3′;
5′-GAU-3′;
5′-AUG-3′;
5′-GCG-3′;
5′-UUC-3′;
5′-GCC-3′;
5′-GGG-3′;
5′-AUU-3′;
5′-GCA-3′;
5′-AGC-3′;
5′-AAC-3′;
5′-CCA-3′;
5′-UGC-3′;
5′-CAA-3′;
5′-CGG-3′;
5′-ACC-3′;
5′-AGA-3′;
5′-TTT-3′;
5′-TCT-3′;

and
a variant of the motifs or sequences thereof having at least about 75% sequence identity thereto;
wherein the U may be a T and/or the T may be a U and wherein the oligonucleotide inhibits TLR7 activity when administered to a subject or in any assay described herein.

In one embodiment, the oligonucleotide comprises, consists essentially of or consists of a sequence of 5′-[A/G]GU[A/C][U/C]C-3′ (SEQ ID NO: 1); 5′-A[G/A][U/G]C[U/C]C-3′ (SEQ ID NO: 2); 5′-A[G/A][U/G]C[U/C]C[U/C][C/A]U-3′ (SEQ ID NO: 212); 5′-GGUAUA-3′ (SEQ ID NO: 4); 5′-UGUUUC-3′ (SEQ ID NO: 5); 5′-UGUGUC-3′ (SEQ ID NO: 6); 5′-CGUGUC-3′ (SEQ ID NO: 8); 5′-GCAGUCTCCATGTCCCAGGC-3′ (SEQ ID NO: 30); 5′-GAUGGTTCCAGTCCCUCUUC-3′ (SEQ ID NO: 38); 5′-AGCAGTCTCCATGTCCCAGG-3′ (SEQ ID NO: 31); 5′-GGGUCTCCTCCACACCCUUC-3′ (SEQ ID NO: 36); 5′-GGUGGCCACAGGCAACGUCA-3′ (SEQ ID NO: 28); 5′-GCCGUTTCCATGTCCCAGGC-3′ (SEQ ID NO: 46); 5′-GCGGUATCCATGTCCCAGGC-3′ (SEQ ID NO: 42); 5′-GCGGUATACAGGTCCCAGGC-3′ (SEQ ID NO: 43); 5′-GCUGUTTCCATGTCCCAGGC-3′ (SEQ ID NO: 44); 5′-GCUGUGTCCATGTCCCAGGC-3′ (SEQ ID NO: 45); 5′-GCCGUGTCCATGTCCCAGGC-3′ (SEQ ID NO: 47); 5′-GCGGUAUCCAUGUCCCAGGC-3′ (SEQ ID NO: 151); 5′-GGUATCCCCCCCCCCCCCCC-3′ (SEQ ID NO: 54); 5′-GUCCCATCCCTTCTGCUGCC-3′ (SEQ ID NO: 145); 5′-UUCUCTCTGGTCCCAUCCCU-3′ (SEQ ID NO: 213); 5′-GUUCAGTCAGATCGCUGGGA-3′ (SEQ ID NO: 214); 5′-AUGACATTTCGTGGCUCCUA-3′ (SEQ ID NO: 215); 5′-UCUCCATGTCCCAGGCCUCC-3′ (SEQ ID NO: 216); 5′-AGUCUCCATGTCCCAGGCCU-3′ (SEQ ID NO: 217); 5′-CCAUGTCCCAGGCCTCCAGU-3′ (SEQ ID NO: 29); 5′-GCAAGGCAGAGAAACUCCAG-3′ (SEQ ID NO: 37); 5′-GGAUUAAAACAGATTAAUAC-3′ (SEQ ID NO: 55); 5′-AGCCGAACAGAAGGAGCGUC-3′ (SEQ ID NO: 40); 5′-UCCGGCCTCGGAAGCUCUCU-3′ (SEQ ID NO: 10); 5′-UCCGGCCTCGGAGTCUCCAU-3′ (SEQ ID NO: 52); 5′-GCGGUATCCATAGTCUCCAU-3′ (SEQ ID NO: 48); 5′-GAUUAAAACAGATTAAUACA-3′ (SEQ ID NO: 165); 5′-UGACAAAACAATAATAACAG-3′ (SEQ ID NO: 167); 5′-CCAACACTTCGTGGGGUCCU-3′ (SEQ ID NO: 160), or a variant thereof having at least about 75% sequence identity thereto, wherein the U may be a T and/or the T may be a U.

In one embodiment, the oligonucleotide comprises, consists essentially of or consists of a sequence of 5′-GUX-3′ or 5′-GAX-3′ wherein X is any nucleotide, 5′-GUC-3′, 5′-GUG-3′, 5′-GUA-3′, 5′-GUU-3′, 5′-GGC-3′, 5′-AUC-3′, 5′-GAG-3′, 5′-GGA-3′, 5′-TTT-3′, 5′-TCT-3′, 5′-GAA-3′; 5′-GAC-3′; 5′-GAU-3′; 5′-AUG-3′; 5′-GCG-3′; 5′-UUC-3′; 5′-GCC-3′; 5′-GGG-3′; 5′-AUU-3′; 5′-GCA-3′; 5′-AGC-3′; 5′-AAC-3′; 5′-CCA-3′; 5′-UGC-3′; 5′-CAA-3′; 5′-CGG-3′; 5′-ACC-3′; 5′-AGA-3′; 5′-TTT-3′ or 5′-TCT-3′, preferably wherein one or two bases are modified bases and one or more internucleotide linkages are a modified backbone, preferably 5′-mGmUmX-3′ or 5′-mGmAmX-3′ wherein X is any nucleotide, 5′-mGmUmC-3′, 5′-mGmUmG-3′, 5′-mGmUmA-3′, 5′-mGmUmU-3′, 5′-mGmGmC-3′, 5′-mAmUmC-3′, 5′-mGmAmG-3′, 5′-mGmGmA-3′, 5′-mTmTmT-3′ or 5-mTmCmT-3′, 5′-mGmAmA-3′; 5′-mGmAmC-3′; 5′-mGmAmU-3′; 5′-mAmUmG-3′; 5′-mGmCmG-3′; 5′-mUmUmC-3′; 5′-mGmCmC-3′; 5′-mGmGmG-3′; 5′-mAmUmU-3′; 5′-mGmCmA-3′; 5′-mAmGmC-3′; 5′-mAmAmC-3′; 5′-mCmCmA-3′; 5′-mUmGmC-3′; 5′-mCmAmA-3′; 5′-mCmGmG-3′; 5′-mAmCmC-3′; 5′-mAmGmA-3′; 5′-mUmUmU-3′, 5′mUmCmU-3′, 5′mTmTm-3′ or 5′-mTmCmT-3′, wherein m is a modified base and/or has a modified backbone, preferably wherein the modified base is 2′OMe and the modified backbone is phosphorothioate.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR7.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR7 and the oligonucleotide inhibits, or does not substantially inhibit, TLR3, TLR9 and/or cGAS activity.

In one embodiment, the oligonucleotide inhibits TLR7 activity and inhibits TLR8 activity, for example an oligonucleotide comprising, consisting essentially of or consisting of a sequence of 5′-GUX-3′ or 5′-GAX-3′ wherein X is any nucleotide, 5′-GUC-3′, 5′-GUG-3′, 5′-GUA-3′, 5′-GUU-3′, or 5′-GAG-3′, 5′-GAC-3′, 5′-GAU-3′, 5′-GAA-3′, preferably 5′-mGmUmX-3′ or 5′-mGmAmX-3′ wherein X is any nucleotide, 5′-mGmUmC-3′, 5′-mGmUmG-3′, 5′-mGmUmA-3′, 5′-mGmUmU-3′, 5′-mGmAmG-3′, 5′-mGmAmC-3′, 5′-mGmAmU-3′, 5′-mGmAmA-3′,wherein m is a modified base and/or has a modified backbone, preferably wherein the modified base is 2′OMe and the modified backbone is phosphorothioate.

Referring to the aforementioned aspects, the oligonucleotide may comprise:
a) a 5′ region comprising bases which are modified and/or which have a modified backbone,
b) a middle region comprising ribonucleic acid, deoxyribonucleic acid, or combination thereof, bases, which optionally have a modified backbone, and
c) a 3′ region comprising bases which are modified and/or which have a modified backbone.

In an embodiment, the middle region is about 10 bases in length.

In an embodiment, the 5′ region and/or the 3′ region are: (a) about 3 bases in length; or (b) about 5 bases in length. In embodiments in which the 5′ region and/or the 3′ region are about 5 bases in length, the bases are suitably 2′-OMe and/or 2′-MOE modified. In embodiments in which the 5′ region and/or the 3′ region are about 3 bases in length, the bases are suitably 2′-LNA modified.

In embodiments in which the motif is 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60), wherein the U may be a T, the motif is suitably at or towards the 5′ and/or 3′ end of the oligonucleotide. In certain embodiments, the motif of 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60), wherein the U may be a T, is at or towards the 5′ end of the oligonucleotide.

In some embodiments, the oligonucleotide comprises, consists essentially of or consists of a sequence of 5′-GGUAU-3′ (SEQ ID NO: 56) or a variant thereof having at least about 75% sequence identity thereto and wherein the U may be a T.

In other embodiments, the oligonucleotide comprises, consists essentially of or consists of a sequence of 5′-GGUA-3′ (SEQ ID NO: 57) or a variant thereof having at least about 75% sequence identity thereto and wherein the U may be a T.

In some embodiments, the oligonucleotide comprises, consists essentially of or consists of a sequence of 5′-GUAU-3′ (SEQ ID NO: 58) or a variant thereof having at least about 75% sequence identity thereto and wherein the U may be a T.

In certain embodiments, the oligonucleotide comprises, consists essentially of or consists of a sequence of 5′-GGU-3′ (SEQ ID NO: 59) or a variant thereof having at least about 75% sequence identity thereto and wherein the U may be a T.

In some embodiments, the oligonucleotide comprises, consists essentially of or consists of a sequence of 5′-GUA-3′ (SEQ ID NO: 60) or a variant thereof having at least about 75% sequence identity thereto and wherein the U may be a T.

In other embodiments, the oligonucleotide comprises, consists essentially of or consists of a sequence of:

5′-mGmGmU*mAmU-3′;
5′-mGmGmU*mA-3′;
5′-mGmU*mAmU-3′;
5′-mGmGmU-3′;
5′-mGmU*mA-3′;

or a variant thereof having at least about 75% sequence identity thereto and wherein the U may be a T and/or the T may be a U and wherein m is a modified base and/or has a modified backbone.

In one further aspect, the invention provides an oligonucleotide comprising a motif or sequence selected from the group consisting of:

5′-CUUCGTGGGGTCCTTUUCAC-3′;
5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′;
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;

and
a variant of the motifs or sequences thereof having at least about 75% sequence identity thereto;
wherein the U may be a T and/or the T may be a U and wherein the oligonucleotide potentiates TLR8 activity when administered to a subject or in any assay described herein.

In one embodiment, the oligonucleotide comprises, consists essentially of or consists of a sequence of 5′-CGX-3′, 5′-AGX-3′, 5′GGX-3′ wherein X is any nucleotide, 5′-UCG-3′; 5′-UCA-3′; 5′-CGG-3′; 5′-UGG-3′; 5′-CGC-3′; 5′-AGG-3′; 5′-GGA-3′; 5′-GGC-3′; 5′-AGA-3′; 5′-CGA-3′; 5′-UAG-3′; 5′-UCU-3′; 5′-AGC-3′; 5′-GGU-3′; 5′-UGA-3′5′-AGU-3′; 5′-ACG-3′; 5′-CGU-3′; 5′-UCC-3′; 5′-GCG-3′; 5′-GGG-3′; 5′-UGU-3′; 5′-UCA-3′; 5′-CUG-3′; 5′-UUG-3′; 5′-UUA-3′ or 5′-UGC-3′; preferably wherein one or two bases are modified bases and/or one or more internucleotide linkages are a modified backbone, more preferably 5′-mUmCmG-3′; 5′-mUmCmA-3′; 5′-mCmGmG-3′; 5′-mUmGmG-3′; 5′-mCmGmC-3′; 5′-mAmGmG-3′; 5′-mGmGmA-3′; 5′-mGmGmC-3′; 5′-mAmGmA-3′; 5′-mCmGmA-3′; 5′-mUmAmG-3′; 5′-mUmCmU-3′; 5′-mAmGmC-3′; 5′-mGmGmU-3′; 5′-mUmGmA-3′5′-mAmGmU-3′; 5′-mAmCmG-3′; 5′-mCmGmU-3′; 5′-mUmCmC-3′; 5′-mGmCmG-3′; 5′-mGmGmG-3′; 5′-mUmGmU-3′; 5′-mUmCmA-3′; 5′-mCmUmG-3′; 5′-mUmUmG-3′; 5′-mUmUmA-3′ or 5′-mUmGmC-3′, wherein m is a modified base and/or has a modified backbone, preferably wherein the modified base is 2′OMe and the modified backbone is phosphorothioate.

In one embodiment, the oligonucleotide comprises, consists essentially of or consists of mC*mU*mU*C*G*T*G*G*G*G*T*C*C*T*T*mU*mU*mC*mA*mC, wherein m is the modified base is 2′OMe and * is the modified backbone phosphorothioate. The oligonucleotide may have a LNA at the first CG.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to potentiate the activity of TLR8.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to potentiate the activity of TLR8 and the oligonucleotide inhibits, or does not substantially inhibit, TLR3, TLR7, TLR9 and/or cGAS activity.

In one further aspect, the invention provides an oligonucleotide comprising a motif or sequence selected from the group consisting of:

5′-GAG-3′;
5′-GAC-3′;
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5′-GCU-3′;
5′-CCU-3′;
5′-CUA-3′;
5′-CUC-3′;
5′-AAC-3′;

and
wherein the U may be a T and/or the T may be a U and wherein the oligonucleotide inhibits TLR8 activity when administered to a subject or in any assay described herein.

In one embodiment, the oligonucleotide comprises, consists essentially of or consists of a sequence of 5′-GAX-3′ or 5′-GUX-3′ wherein X is any nucleotide, 5′-GAG-3′, 5′-GAC-3′, 5′-GAU-3′, 5′-GAA-3′, 5′-GUC-3′, 5′-GUU-3′, 5′-GUA-3′, 5′-GUG-3′, 5′-AUA-3′; 5′-AUG-3′; 5′-CUU-3′; 5′-AAG-3′; 5′-AUC-3′; 5′-CCC-3′; 5′-GCU-3′; 5′-CCU-3′; 5′-CUA-3; 5′-CUC-3 or 5′-AAC-3′; preferably wherein one or two bases are modified bases and/or one or more internucleotide linkages are modified backbone, more preferably 5′-mGmAmX-3′ or 5′-mGmUmX-3′ wherein X is any nucleotide, 5′-mGmAmG-3′, 5′-mGmAmC-3′, 5′-mGmAmU-3′, 5′-mGmAmA-3′, 5′-mGmUmC-3′, 5′-mGmUmU-3′, 5′-mGmUmA-3′, 5′-mGmUmG-3′, 5′-mAmUmA-3′; 5′-mAmUmG-3′; 5′-mCmUmU-3′; 5′-mAmAmG-3′; 5′-mAmUmC-3′; 5′-mCmCmC-3′; 5′-mGmCmU-3′; 5′-mCmCmU-3′; 5′-mCmUmA-3; 5′-mCmUmC-3 or 5′-mAmAmC-3 wherein m is a modified base and/or has a modified backbone, preferably wherein the modified base is 2′OMe and the modified backbone is phosphorothioate.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR8.

In one embodiment, the oligonucleotide comprises, consists essentially of or consists of any sequence in Tables 1, 1A, 2, 2A, 3, 3A, 4, 4A, 5, 5A or 6, with or without the particular modifications defined, that is shown in the Examples to inhibit the activity of TLR8 and the oligonucleotide inhibits, or does not substantially inhibit, TLR3, TLR7, TLR9 and/or cGAS activity.

In one further aspect, the invention provides an oligonucleotide comprising a motif or sequence selected from the group consisting of:

5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′;
5′-TAG-3′;
5′-TGG-3′;
5′-TCA-3′;
5′-TGC-3′;
5′-CAC-3′;
5′-CGG-3′;
5′-CCC-3′;
5′-ACT-3′;
5′-GTA-3′;
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;

and
wherein the U may be a T and/or the T may be a U and wherein the oligonucleotide inhibits TLR3 activity when administered to a subject or in any assay described herein.

In one embodiment, the oligonucleotide comprises, consists essentially of or consists of a sequence of 5′-TAC-3′, 5′-CGC-3′, 5′-GCA-3′, 5′-UGA-3′, 5′-CAG-3′, 5′-UGG-3′, or 5′-UCA-3′, preferably 5′-mTmAmC-3′, 5′-mCmGmC-3′, 5′-mGmCmA-3′, 5′-mUmGmA-3′, 5′-mCmAmG-3′, 5′-mUmGmG-3′, 5′-mUmCmA-3′, wherein m is a modified base and/or has a modified backbone, preferably wherein the modified base is 2′OMe and the modified backbone is phosphorothioate.

In one embodiment, the oligonucleotide comprises, consists essentially of or consists of any sequence in Tables 1, 1A, 2, 2A, 3, 3A, 4, 4A, 5, 5A, 6 or 7, with or without the particular modifications defined, that is shown in the Examples to inhibit the activity of TLR3.

In one embodiment, the oligonucleotide comprises, consists essentially of or consists of any sequence in Tables 1, 1A, 2, 2A, 3, 3A, 4, 4A, 5, 5A, 6 or 7, with or without the particular modifications defined, that is shown in the Examples to inhibit the activity of TLR3 and the oligonucleotide inhibits, or does not substantially inhibit, TLR8, TLR7, TLR9 and/or cGAS activity.

In still another aspect, the invention provides a composition comprising, consisting essentially of or consisting of an oligonucleotide of the above aspects or embodiments.

In an embodiment, the composition further comprises a pharmaceutically or physiologically acceptable carrier.

In an embodiment, the composition consists essentially of an oligonucleotide of the above aspects or embodiments and a pharmaceutically acceptable carrier.

In an embodiment, the composition further comprises a non-toxic pharmaceutically or physiologically acceptable carrier.

In an embodiment, the only active pharmaceutical ingrediment present in the composition is an oligonucleotide of the above aspects or embodiments. Preferably, prior to use, i.e. prior to administration to a subject or prior to contacting a cell, the only active pharmaceutical ingrediment present in the composition is an oligonucleotide of the above aspects or embodiments In any embodiment, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% of the oligonucleotide content of the composition is an oligonucleotide of any of the above aspects or embodiments. Preferably, prior to use, i.e. prior to administration to a subject or prior to contacting a cell, the composition has the above oligonucleotide content.

In any embodiment, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% of the oligonucleotide content of the composition is an oligonucleotide of any of the above aspects or embodiments. Preferably, prior to use, i.e. prior to administration to a subject or prior to contacting a cell, the composition has the above oligonucleotide content.

In any embodiment, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% of the active pharmaceutical ingrediment in the composition is an oligonucleotide of any of the above aspects or embodiments.

In a further aspect, the invention relates to a method of reducing expression of a target gene in a cell, the method comprising contacting the cell with an oligonucleotide or composition of the above aspects.

In yet a further aspect, the invention resides in a method of treating or preventing a disease, disorder or condition in a subject, the method comprising administering to the subject a therapeutically effective amount of an oligonucleotide or composition of the above aspects to thereby treat or prevent the disease, disorder or condition in the subject.

In a related aspect, the invention provides a use of an oligonucleotide or composition of the above aspects in the manufacture of a medicament for treating or preventing a disease, disorder or condition in a subject.

In another related aspect, the invention relates to an oligonucleotide or composition of the above aspects for use in treating or preventing a disease, disorder or condition in a subject.

In one embodiment of the three above aspects, the oligonucleotide or composition reduces the expression of a target gene involved in the disease, disorder or condition.

Suitably, the disease, disorder or condition of the three aforementioned aspects demonstrates increased, excessive or abnormal cGAS expression, activity and/or signalling.

In an embodiment, the disease, disorder or condition is selected from the group consisting of Huntington's disease, Parkinson's diseases, motor-neurone disease (MND), amyotrophic lateral sclerosis (ALS), prion disease, frontotemporal dementia, Traumatic brain injury, Alzheimer's disease, Acute pancreatitis, Silica-induced fibrosis, Age dependent macular degeneration, Aicardi-Goutibres syndrome, myocardial infarction, heart failure, Polyarthritis/foetal and neonatal anaemia, Systemic lupus erythematosus, Acute Kidney Injury, Alcohol-related liver disease, Non-Alcohol-fatty liver disease, silica driven lung inflammation, chronic obstructive pulmonary disease, brain injury after ischemic stroke, sepsis, Non-alcoholic steatohepatitis (NASH), cancer, sickle cell disease, Inflammatory bowel disease, type 2 diabetes mellitus, over-nutrition-induced obesity, COVID-19, chronic obstructive pulmonary disorder (COPD), hematopoietic disorders, aging-associated inflammation, Cutibacterium acnes Infection, Hepatitis B, posterior-segment eye diseases, arthritis, rheumatoid arthritis, emphysema, colorectal cancer, skin cancer, metastases, and breast cancer.

In some embodiments, the disease, disorder or condition of the three aforementioned aspects is a senescence-associated disease, disorder or condition, such as aging and/or an aging-related disease, disorder or condition. In this regard, the oligonucleotide suitably inhibits cGAS activity when administered to the subject.

Suitably, the disease, disorder or condition of the three aforementioned aspects demonstrates increased, excessive or abnormal TLR9 expression, activity and/or signalling. In an embodiment, the disease, disorder or condition is selected from the group consisting of psoriasis, rheumatoid arthritis, alopecia universalis, acute disseminated encephalomyelitis, Addison's disease, allergy, ankylosing spondylitis, antiphospholipid antibody syndrome, arteriosclerosis, atherosclerosis, autoimmune hemolytic anemia, autoimmune hepatitis, Bullous pemphigoid, Chagas' disease, chronic obstructive pulmonary disease, coeliac disease, cutaneous lupus erythematosus (CLE), dermatomyositis, diabetes, dilated cardiomyopathy (DC), endometriosis, Goodpasture's syndrome, Graves' disease, Guillain-Barre syndrome, Hashimoto's disease, hidradenitis suppurativa, idiopathic thrombocytopenic purpura, inflammatory bowel disease, interstitial cystitis, morphea, multiple sclerosis (MS), myasthenia gravis, myocarditis, narcolepsy, neuromyotonia, pemphigus, pernicious anaemia, polymyositis, primary biliary cirrhosis, rheumatoid arthritis (RA), schizophrenia, Sjogren's syndrome, systemic lupus erythematosus (SLE), systemic sclerosis, temporal arteritis, vasculitis, vitiligo, vulvodynia, Wegener's granulomatosis, traumatic pain, neuropathic pain and acetaminophen toxicity, breast cancer, cervical squamous cell carcinoma, gastric carcinoma, glioma, hepatocellular carcinoma, lung cancer, melanoma, prostate cancer, recurrent glioblastoma, recurrent non-Hodgkin lymphoma and colorectal cancer.

In one embodiment, the disease, disorder or condition demonstrates increased, excessive or abnormal TLR7 expression, activity and/or signalling.

In one embodiment, the disease, disorder or condition of the three aforementioned aspects demonstrates increased, excessive or abnormal TLR8 expression, activity and/or signalling.

In one embodiment, the disease, disorder or condition of the three aforementioned aspects demonstrates increased, excessive or abnormal TLR3 expression, activity and/or signalling.

In other embodiments, the disease, disorder or condition of the three aforementioned aspects is an inflammatory disease, disorder or condition associated with administration of a therapeutic oligonucleotide to the subject. In this regard, the inflammatory disease, disorder or condition is associated at least in part with activation of one or more nucleic acid sensors, such as cGAS, TLR3, TLR8, TLR9 and/or TLR7, following administration of the therapeutic oligonucleotide. In one embodiment, the inflammatory disease, disorder or condition comprises hepatic inflammation.

In a further aspect, the invention resides in a method of inhibiting cGAS in a subject, including the step of administering to the subject an effective amount of an oligonucleotide or composition of the above aspects.

In a related aspect, the invention resides in a method of inhibiting cGAS in a cell, including the step of contacting the cell with an effective amount of an oligonucleotide or composition of the above aspects.

In particular embodiments of the above two aspects, the oligonucleotide comprises, consists of or consists essentially of the motif selected from 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GUA-3′ (SEQ ID NO: 60) and 5′-GGU-3′ (SEQ ID NO: 59), wherein the U may be a T. In such examples, the motif is suitably at or towards a 5′ end of the oligonucleotide.

In an embodiment, the oligonucleotide inhibits or prevents senescence in the cell.

In an embodiment, the cell is an immune cell.

In an embodiment, the cell is in a cell culture. In an embodiment, the method comprises culturing the cells. In an embodiment, the method produces more live cells than cells cultured under identical conditions but lacking the oligonucleotide. Thus, the method can be used to produce a given number of cells with fewer passages.

In one embodiment of the above aspects, the oligonucleotide comprises, consists essentially of or consists of the sequence of:

(SEQ ID NO: 30)
5′-GCAGUCTCCATGTCCCAGGC-3′;
(SEQ ID NO: 38)
5′-GAUGGTTCCAGTCCCUCUUC-3′;
(SEQ ID NO: 31)
5′-AGCAGTCTCCATGTCCCAGG-3′;
(SEQ ID NO: 36)
5′-GGGUCTCCTCCACACCCUUC-3′;
(SEQ ID NO: 28)
5′-GGUGGCCACAGGCAACGUCA-3′;
(SEQ ID NO: 46)
5′-GCCGUTTCCATGTCCCAGGC-3′;
(SEQ ID NO: 42)
5′-GCGGUATCCATGTCCCAGGC-3′;
(SEQ ID NO: 43)
5′-GCGGUATACAGGTCCCAGGC-3′;
(SEQ ID NO: 44)
5′-GCUGUTTCCATGTCCCAGGC-3′;
(SEQ ID NO: 45)
5′-GCUGUGTCCATGTCCCAGGC-3′;
(SEQ ID NO: 47)
5′-GCCGUGTCCATGTCCCAGGC-3′;
(SEQ ID NO: 151)
5′-GCGGUAUCCAUGUCCCAGGC-3′;
(SEQ ID NO: 54)
5′-GGUATCCCCCCCCCCCCCCC-3′;
(SEQ ID NO: 27)
5′-CUUGUGAAAAGATTAUCUUC-3′;
(SEQ ID NO: 29)
5′-CCAUGTCCCAGGCCTCCAGU-3′;
(SEQ ID NO: 37)
5′-GCAAGGCAGAGAAACUCCAG-3′;
(SEQ ID NO: 55)
5′-GGAUUAAAACAGATTAAUAC-3′;
(SEQ ID NO: 40)
5′-AGCCGAACAGAAGGAGCGUC-3′;
(SEQ ID NO: 10)
5′-UCCGGCCTCGGAAGCUCUCU-3′;
(SEQ ID NO: 52)
5′-UCCGGCCTCGGAGTCUCCAU-3′;
(SEQ ID NO: 48)
5′-GCGGUATCCATAGTCUCCAU-3′;

or a variant thereof having at least about 75% sequence identity thereto, wherein the U may be a T and/or the T may be a U.

In one embodiment, the oligonucleotide comprises, consists essentially of or consists of the sequence of 5′-GCGGUATCCATGTCCCAGGC-3′ (SEQ ID NO: 42) or a variant thereof having at least about 75% sequence identity thereto, wherein the U may be a T and/or the T may be a U.

In another embodiment, the oligonucleotide comprises, consists essentially of or consists of the sequence of 5′-mGmCmGmGmUATCCATGTCCmCmAmGmGmC-3′, or a variant thereof having at least about 75% sequence identity thereto, wherein the U may be a T and/or the T may be a U and wherein m is a modified base and/or has a modified backbone.

In another embodiment, the oligonucleotide comprises, consists essentially of or consists of the sequence of 5′-mGmCmGmGmUmAmTmCmCmAmTmGmTmCmCmCmAmGmGmC-3′, or a variant thereof having at least about 75% sequence identity thereto, wherein the U may be a T and/or the T may be a U and wherein m is a modified base and/or has a modified backbone.

In still a further aspect, the invention provides a method of inhibiting TLR9 in a subject, including the step of administering to the subject an effective amount of an oligonucleotide or composition of the above aspects.

In a related aspect, the invention resides in a method of inhibiting TLR9 in a cell, including the step of contacting the cell with an effective amount of an oligonucleotide or composition of the above aspects.

With respect to the above two aspects, the oligonucleotide suitably inhibits TLR9 activation in the cell or the subject in response to an RNA molecule, such as an exogenous RNA molecule.

In one embodiment of the above aspects, the oligonucleotide comprises, consists essentially of or consists of a sequence of 5′-UCCGGCCTCGGAGTCUCCAU-3′ (SEQ ID NO: 52), 5′-GCGGUATCCATAGTCUCCAU-3′ (SEQ ID NO: 48), 5′-CCAACACTTCGTGGGGUCCU-3′ (SEQ ID NO: 160), 5′-CACUUCGTGGGGTCCUUUUC-3′ (SEQ ID NO: 159), 5′-UCCGGCCTCGGAAGCUCUCU-3′ (SEQ ID NO: 10), 5′-GAUUAAAACAGATTAAUACA-3′ (SEQ ID NO: 165), 5′-UGACAAAACAATAATAACAG-3′ (SEQ ID NO: 167); 5′-UCCGGCCTCGGAAGCUCUCU-3′ (SEQ ID NO: 10); or a variant thereof having at least about 75% sequence identity thereto and wherein the U may be a T and/or the T may be a U.

In one embodiment, the oligonucleotide comprises, consists essentially of or consists of the sequence of 5′-UCCGGCCTCGGAAGCUCUCU-3′ (SEQ ID NO: 10) or a variant thereof having at least about 75% sequence identity thereto wherein the U may be a T and/or the T may be a U.

In another embodiment, the oligonucleotide comprises, consists essentially of or consists of the sequence of 5′-mUmCmCmGmGCCTCGGAAGCmUmCmUmCmU-3′ or a variant thereof having at least about 75% sequence identity thereto, wherein the U may be a T and/or the T may be a U and wherein m is a modified base and/or has a modified backbone.

In another aspect, the invention resides in a method of inhibiting TLR7 in a subject, including the step of administering to the subject an effective amount of an oligonucleotide or composition of the above aspects.

In a related aspect, the invention provides a method of inhibiting TLR7 in a cell, including the step of contacting the cell with an effective amount of an oligonucleotide or composition of the above aspects.

With respect to the above two aspects, the oligonucleotide or composition suitably inhibits TLR7 activation in the cell or the subject in response to an RNA molecule, such as an exogenous RNA molecule, or TLR7 agonist such as a small molecule.

In another related aspect, the invention relates to a method of preventing or inhibiting TLR7 activation by an RNA molecule in a cell, said method including the step of contacting the cell with an effective amount of an oligonucleotide of the above aspects.

In a further related aspect, the invention resides in a method of preventing or inhibiting TLR7 activation by an RNA molecule or TLR7 agonist in a subject, said method including the step of administering to the subject an effective amount of an oligonucleotide of the above aspects.

In still a further aspect, the invention provides a method of inhibiting TLR8 in a subject, including the step of administering to the subject an effective amount of an oligonucleotide of the above aspects.

In a related aspect, the invention resides in a method of inhibiting TLR8 in a cell, including the step of contacting the cell with an effective amount of an oligonucleotide of the above aspects.

With respect to the above two aspects, the oligonucleotide or composition suitably inhibits TLR8 activation in the cell or the subject in response to an RNA molecule, such as an exogenous RNA molecule, or a TLR8 agonist such as a small molecule, for example Motolimod.

In another related aspect, the invention relates to a method of preventing or inhibiting TLR8 activation by an RNA molecule or TLR8 agonist in a cell, said method including the step of contacting the cell with an effective amount of an oligonucleotide or composition of the above aspects.

In a further related aspect, the invention resides in a method of preventing inhibiting TLR8 activation by an RNA molecule in a subject, said method including the step of administering to the subject an effective amount of an oligonucleotide or composition of the above aspects.

In still a further aspect, the invention provides a method of inhibiting TLR3 in a subject, including the step of administering to the subject an effective amount of an oligonucleotide of the above aspects.

In a related aspect, the invention resides in a method of inhibiting TLR3 in a cell, including the step of contacting the cell with an effective amount of an oligonucleotide of the above aspects.

With respect to the above two aspects, the oligonucleotide or composition suitably inhibits TLR3 activation in the cell or the subject in response to an RNA molecule, such as an exogenous RNA molecule, or a TLR3 agonist such as a small molecule.

In another related aspect, the invention relates to a method of preventing or inhibiting TLR3 activation by an RNA molecule or TLR3 agonist in a cell, said method including the step of contacting the cell with an effective amount of an oligonucleotide or composition of the above aspects.

In a further related aspect, the invention resides in a method of preventing inhibiting TLR3 activation by an RNA molecule or TLR3 agonist in a subject, said method including the step of administering to the subject an effective amount of an oligonucleotide or composition of the above aspects.

In still a further aspect, the invention provides a method of increasing the activity of, or potentiating, TLR8 in a subject, including the step of administering to the subject an effective amount of an oligonucleotide of the above aspects.

In a related aspect, the invention resides in a method of increasing the activity of, or potentiating, TLR8 in a cell, including the step of contacting the cell with an effective amount of an oligonucleotide of the above aspects.

With respect to the above two aspects, the oligonucleotide or composition suitably increases TLR8 activation in the cell or the subject in response to an RNA molecule, such as an exogenous RNA molecule, or a TLR8 agonist such as a small molecule, for example Motolimod. Therefore, the potentiation of TLR8 activity by an oligonucleotide of the invention in the presence of a co-administered TLR8 agonist allows a lower dose of the TLR8 agonist to be administered invention. Other TLR7/8 agonists that can be potentiated include R848, Loxoribine, gardiquimod, Isatoribine, Imiquimod, CL075, CL097, CL264, CL307, 852A, or TL8-506.

In another related aspect, the invention relates to a method of increasing or potentiating TLR8 activation by an RNA molecule in a cell, said method including the step of contacting the cell with an effective amount of an oligonucleotide or composition of the above aspects.

In a further related aspect, the invention resides in a method of increasing or potentiating TLR8 activation by an RNA molecule in a subject, said method including the step of administering to the subject an effective amount of an oligonucleotide or composition of the above aspects.

In particular embodiments, the RNA molecule is a messenger RNA (mRNA) molecule. Suitably, the mRNA molecule is a component of or included within an immunogenic composition, such as an mRNA vaccine composition.

In another aspect, the invention provides an immunogenic composition comprising an RNA molecule and an oligonucleotide of the above aspects.

Suitably, the immunogenic composition is an mRNA vaccine composition.

In one embodiment of the above aspects, the oligonucleotide comprises, consists essentially of or consists of a sequence of 5′-GCAGUCTCCATGTCCCAGGC-3′ (SEQ ID NO: 30); 5′-GAUGGTTCCAGTCCCUCUUC-3′ (SEQ ID NO: 38); 5′-AGCAGTCTCCATGTCCCAGG-3′ (SEQ ID NO: 31); 5′-GGGUCTCCTCCACACCCUUC-3′ (SEQ ID NO: 36); 5′-GGUGGCCACAGGCAACGUCA-3′ (SEQ ID NO: 28); 5′-GCCGUTTCCATGTCCCAGGC-3′ (SEQ ID NO: 46); 5′-GCGGUATCCATGTCCCAGGC-3′ (SEQ ID NO: 42); 5′-GCGGUATACAGGTCCCAGGC-3′ (SEQ ID NO: 43); 5′-GCUGUTTCCATGTCCCAGGC-3′ (SEQ ID NO: 44); 5′-GCUGUGTCCATGTCCCAGGC-3′ (SEQ ID NO: 45); 5′-GCCGUGTCCATGTCCCAGGC-3′ (SEQ ID NO: 47); 5′-GCGGUAUCCAUGUCCCAGGC-3′ (SEQ ID NO: 151); 5′-GGUATCCCCCCCCCCCCCCC-3′ (SEQ ID NO: 54); 5′-GUCCCATCCCTTCTGCUGCC-3′ (SEQ ID NO: 145); 5′-UUCUCTCTGGTCCCAUCCCU-3′ (SEQ ID NO: 213); 5′-GUUCAGTCAGATCGCUGGGA-3′ (SEQ ID NO: 214); 5′-AUGACATTTCGTGGCUCCUA-3′ (SEQ ID NO: 215); 5′-UCUCCATGTCCCAGGCCUCC-3′ (SEQ ID NO: 216); 5′-AGUCUCCATGTCCCAGGCCU-3′ (SEQ ID NO: 217); or a variant thereof having at least about 75% sequence identity thereto and wherein the U may be a T and/or the T may be a U.

In particular embodiments of the above five aspects, the oligonucleotide comprises, consists of or consists essentially of the motif selected from 5′-GGUAUA-3′ (SEQ ID NO: 4), 5′-GGUAU-3′ (SEQ ID NO: 56), 5′-GGUA-3′ (SEQ ID NO: 57), 5′-GUAU-3′ (SEQ ID NO: 58), 5′-GUA-3′ (SEQ ID NO: 60) and 5′-GGU-3′ (SEQ ID NO: 59), wherein the U may be a T. In such examples, the motif is suitably at or towards a 5′ end of the oligonucleotide.

Suitably for the aforementioned aspects, one or more of the bases of the motif are a modified base and/or have a modified backbone, such as those described herein.

Examples of modified bases useful for the invention include, but are not limited to, those which comprise a 2′-O-methyl, 2′-O-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino, fluoroarabinonucleotide, threose nucleic acid or 2′-O-(N-methlycarbamate).

Referring to the above aspects, the modified backbone suitably comprises a phosphorothioate, a non-bridging oxygen atom substituting a sulfur atom, a phosphonate such as a methylphosphonate, a phosphodiester, a phosphoromorpholidate, a phosphoropiperazidate, amides, methylene(methylamino), fromacetal, thioformacetal, a peptide nucleic acid or a phosphoroamidate such as a morpholino phosphorodiamidate (PMO), N3′-P5′ phosphoramidite or thiophosphoroamidite.

For the above aspects, at least a portion of the oligonucleotide suitably has/is a ribonucleic acid, deoxyribonucleic acid, DNA phosphorothioate, RNA phosphorothioate, 2′-O-methyl-oligonucleotide, 2′-O-methyl-oligodeoxyribonucleotide, 2′-O-hydrocarbyl ribonucleic acid, 2′-O-hydrocarbyl DNA, 2′-O-hydrocarbyl RNA phosphorothioate, 2′-O-hydrocarbyl DNA phosphorothioate, 2′-F-phosphorothioate, 2′-F-phosphodiester, 2′-methoxyethyl phosphorothioate, 2-methoxyethyl phosphodiester, deoxy methylene(methylimino) (deoxy MMI), 2′-O-hydrocarby MMI, deoxy-methylphos-phonate, 2′-O-hydrocarbyl methylphosphonate, morpholino, 4′-thio DNA, 4′-thio RNA, peptide nucleic acid, 3′-amidate, deoxy 3′-amidate, 2′-O-hydrocarbyl 3′-amidate, locked nucleic acid, cyclohexane nucleic acid, tricycle-DNA, 2′fluoro-arabino nucleic acid, N3′-P5′ phosphoroamidate, carbamate linked, phosphotriester linked, a nylon backbone modification and any combination thereof.

In an embodiment of the above aspects, the modified base comprises:
(a) a 2′O-methyl and a phosphorothioate backbone;
(b) a 2′-LNA and a phosphorothioate backbone; or
(c) a 2′-O-methoxyethoxy and a phosphorothioate backbone.

In an embodiment of the above aspects, at least one of the bases of the oligonucleotide does not hybridize to a target polynucleotide.

Suitably, the oligonucleotide of the above aspects is an antisense oligonucleotide, such as a gapmer antisense oligonucleotide, or a double stranded oligonucleotide for gene silencing, such as an siRNA or an shRNA. In certain embodiments, one or more bases of the motif or the oligonucleotide are removed by an endonuclease in vivo.

In alternative embodiments, the oligonucleotide of the invention is a synthetic oligonucleotide. In this regard, the oligonucleotide is designed to not bind or hybridize to a target polynucleotide, such as a cellular or naturally occurring transcript.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Sequence-dependent inhibition of cGAS sensing by ASOs. (A) HeLa and HT-29 cells were transfected for 24 h with 20 nM of indicated ASOs targeted to cGAS (Table 1), prior to RNA purification and RT-qPCR analyses. cGASlevels were reported relative to 18S, and normalised to Mock condition. Data shown represent the median of two independent experiments for each cell line. (B) THP-1 pre-treated overnight with 100 nM of the indicated ASO, were transfected or not (non-treated [NT]) with 2.5 μg/ml ISD70 for 8.5 h and IP-10 levels in supernatants determined by ELISA. Data shown are averaged from two independent experiments in biological triplicate (±s.e.m and ordinary one-way ANOVA with Tukey's multiple comparison tests to the “ISD70 only” condition, or otherwise indicated pairs of conditions are shown). There was no basal effect of the ASOs on NT cells (Alharbi et al., 2020). (C) HT-29 cells pre-treated overnight with 125, 250 or 500 nM of indicated ASOs, were transfected or not (non-treated [NT]) with 2.5 μg/ml of ISD70 for 24 h, and IP-10 levels in supernatants determined by ELISA. IP-10 levels were normalised to the “ISD70 only” condition, after background correction with NT condition. Data shown are averaged from two independent experiments in biological triplicate (I s.e.m and Mann-Whitney U tests to the “ISD70 only” condition are shown). (D, E) THP-1 pre-treated overnight with 100 nM indicated ASOs, were transfected with 2.5 μg/ml of ISD70 for 7-8 h, and IP-10 levels in supernatants determined by ELISA. (D) Stimulations and ELISAs were carried out in two independent plates, and the results presented on each axes (with a correlation r=0.7716, P≤0.0001). Averaged values from both plates are given in Table 2. ISD70 only condition is shown in blue. (E) IP-10 levels were normalised to the “ISD70 only” condition, after background correction with NT condition. Data shown are averaged from three independent experiments in biological triplicate (±s.e.m and ordinary one-way ANOVA with Tukey's multiple comparison tests to the condition “ISD70 only”, or otherwise indicated pairs of conditions are shown). (F) HT-29 cells pre-treated overnight with 187.5 nM of indicated ASOs were transfected or not with 2.5 μg/ml of ISD70 for 24 h, and IP-10 levels in supernatants determined by ELISA. IP-10 levels were normalised to the “ISD70 only” condition, after background correction with NT condition. Data shown are averaged from three independent experiments in biological triplicate (±s.e.m and ordinary one-way ANOVA with Dunnett's multiple comparison tests to the condition “ISD70 only” condition are shown). (G) cGAS−/−, UNC93B1−/− and matched controls with rescued UNC93B1 expression (UNC93B1 WT) THP-1 were pre-treated 6 h with 100 or 250 nM ASOs, and transfected with 2.5 μg/ml of ISD70 overnight. GSK (100 nM) and ODN2006 (500 nM) were used as human STING and TLR9 agonists, respectively. IP-10 levels in supernatants were determined by ELISA and normalised to the “GSK” condition, after background correction with NT condition. Data shown are averaged from three independent experiments in biological triplicate (±s.e.m and ordinary two-way ANOVA with Tukey's multiple comparison tests relative to “ISD70 only” condition are shown). * P≤0.05, ** P≤0.01, *** P≤0.001, **** P≤0.0001, ns: non-significant.

FIG. 2. Identification of a highly potent inhibitor of cGAS sensing. (A) Top: sequence alignments of cGAS inhibitors, and identification of important bases predicted by MEME analysis, highlighted with arrows (FIG. 6A). Bottom: design of C2 and ASO2 mutants incorporating point mutations at selected positions of the MEME motif (mutations are highlighted in yellow). (B, D) HT-29 cells pre-treated overnight with indicated doses of ASOs (500, 250, 125, 62.5 nM), were transfected or not with 2.5 μg/ml of ISD70 for 24 h and IP-10 levels in supernatants determined by ELISA. IP-10 levels were normalised to the “ISD70 only” condition, after background correction with NT condition. Data shown are averaged from two (D) or three (B) independent experiments in biological triplicate (±s.e.m and ordinary two-way ANOVA with Tukey's multiple comparison tests relative to C2 [B] or ASO2 [D] conditions, are shown; comparisons were not significant between the ASOs for 250 and 500 nM). (C) Mouse LL171 cells were treated for 6 h with indicated amount of ASOs (200, 400, 600 nM) prior to overnight stimulation with 2.5 μg/ml ISD45. Cells were lysed and ISRE-Luc levels were analysed by luciferase assay the next day. ISRE-luciferase levels were normalised to “ISD45 only” condition, after background correction with NT condition. Data shown are averaged from two independent experiments in biological triplicate (±s.e.m and Mann-Whitney U tests are shown). (E) Top: sequence alignments of C2-Mut1 variants; C2-ASO2-A and C2-ASO2-B have the 3′ ends from ASO2up and ASO2down underlined. Bottom: variants of the homopolymer dC20; 2′OMe bases are in pink and the cGAS inhibitory motif underlined. (F) HT-29 cells pre-treated overnight with 187.5 nM indicated ASOs were transfected or not with 2.5 μg/ml of ISD70 for 24 h and IP-10 levels in supernatants determined by ELISA. (G) THP-1 pre-treated overnight with 100 nM indicated ASOs were transfected with 2.5 μg/ml of ISD70 for 24 h and IP-10 levels in supernatants determined by ELISA. (F, G) IP-10 levels were normalised to the “ISD70 only” condition, after background correction with NT condition. Data shown are averaged from three independent experiments in biological triplicate (±s.e.m and ordinary one-way ANOVA with Tukey's multiple comparison tests relative to condition “ISD70 only”, or otherwise indicated pairs of conditions are shown). (H) LL171 cells were treated for 6 h with 200 nM ASOs prior to overnight stimulation with 2.5 μg/ml ISD45. Cells were lysed and ISRE-Luc levels were analysed by luciferase assay the next day. ISRE-luciferase levels were normalised to “ISD45 only” condition, after background correction with NT condition. Data shown are averaged from three independent experiments in biological triplicate (±s.e.m and ordinary one-way ANOVA with Dunnett's multiple comparison tests to the “ISD70 only” condition are shown). (I) THP-1 cells were treated for 6 h with 100 nM C2-Mut1, or 100, 250, 500 nM of C2-Mut1-PS were transfected with 2.5 μg/ml of ISD70 overnight. IP-10 levels were normalised to the “ISD70 only” condition, after background correction with NT condition. Data shown are averaged from two independent experiments in biological triplicate (±s.e.m and Mann-Whitney U tests to the “ISD70 only” condition are shown). (J) Sequences of [LINC-PINT] ASOs 101-116, showing the location of the inhibitory GGUCCC motif (in pink) from ASO103 identified by MEME (see D and FIG. 6B). The yellow region highlights the DNA moiety of the gapmers. (K) THP-1 pre-treated overnight with 100 nM indicated [LINC-PINT] ASOs, were transfected with 2.5 μg/ml of ISD70 for 7.5 h and IP-10 levels in supernatants determined by ELISA. IP-10 levels were normalised to the “ISD70 only” condition, after background correction with NT condition. Data shown are averaged from three independent experiments in biological triplicate (I s.e.m and one-way ANOVA with Dunnett's multiple comparison tests to the “ISD70 only” condition are shown). (L, M) LL171 cells were treated for 6 h or 20 min with 200 nM ASOs prior to being “washed” or not (L), or treated with 50, 100 or 200 nM ASOs for 20 min (M), and stimulated overnight with 2.5 μg/ml ISD45. Cells were lysed and ISRE-Luc levels were analysed by luciferase assay the next day. ISRE-luciferase levels were normalised to “ISD45 only” condition, after background correction with NT condition. Data shown are averaged from two (L) or three (M) independent experiments in biological triplicate (±s.e.m and one-way ANOVA with Tukey's multiple comparison tests to the “ISD45 only” condition (L), or Mann-Whitney U tests to the “ISD45 only” (M), or otherwise indicated pairs of conditions, are shown). (N) THP-1 pre-treated for 40 min with 250 nM indicated ASOs, were transfected overnight with 2.5 μg/ml of ISD70 and IP-10 levels in supernatants determined by ELISA. Data shown are averaged from three independent experiments in biological triplicate (±s.e.m and ordinary one-way ANOVA with Dunnett's multiple comparison tests to the condition “ISD70 only” condition, and a Mann-Whitney U test comparing Mutl-dC and dC20 are shown). * P≤0.05, ** P≤0.01, *** P≤0.001, P≤0.0001, ns: non-significant.

FIG. 3. Sequence-dependent inhibition of cGAS function. (A, B) THP-1 pre-treated 6 h with indicated doses (500, 250, 125, 62.5, 31.25 nM) (A) or 125 nM (B) of C2-Mutl or A151 oligonucleotides, were transfected with 2.5 μg/ml of ISD70 overnight, and IP-(A) or IFN-β (B) levels in supernatants determined by ELISA. IP-10 (A) and IFN-β (B) levels were normalised to the “ISD70 only” condition, after background correction with NT condition. (A, B) Data shown are averaged from three independent experiments in biological triplicate (±s.e.m and ordinary two-way ANOVA with Sidak's multiple comparison tests relative to A151 are shown [A] or ordinary one-way ANOVA with Tukey's multiple comparison tests relative to condition “NT”, or otherwise indicated pairs of conditions are shown [B]). MG-63 (C) or mouse immortalised BMDMs (D) were pre-treated or not with 500 nM for 6 h prior to stimulated with 2.5 μg/ml ISD (ISD70 for MG-63, ISD45 for BMDMs), LPS (1 μg/ml), GSK (100 nM), PAM3C (100 ng/ml), ODN1826 (500 nM), or DMXAA (50 μg/ml) overnight. IP-10 (C and D) and TNF-α (D) levels in supernatants were determined by ELISA. (C) Data were normalised to the “GSK” condition, after background correction with NT condition. Data shown are averaged from two independent experiments in biological triplicate (±s.e.m and Mann-Whitney U tests are shown). (D) IP-10 levels were normalised to the DMXAA condition, while TNF-α were normalised to the LPS condition. Data shown are averaged from three independent experiments in biological triplicate (±s.e.m and Mann-Whitney U tests are shown). (E) Recombinant cGAS protein was incubated in vitro with 2.3 μM ISD70 with or without (NT) increasing concentrations of ASOs (0.5, 2 and 10 μM) for 40 min. The reaction was stopped with EDTA and cGAMP levels analysed by ELISA. Data were normalised to the condition A151 2 μM. Data shown are averaged from three independent experiments ran in technical duplicate on the ELISA (±s.e.m and a Mann-Whitney U test is shown). (F) BJ hTERT SV40T cells were transfected overnight with increasing amount of indicated ASO (20, 50, 100 nM), or 2 mM aspirin (ASP), prior to RNA purification. Expression of the panel of 3 human IFN-driven genes was analysed by RT-qPCR. Expression of the indicated genes was reported to 18S expression and further normalised to the average of the “Mock” condition. Data shown represent the average of three independent experiments conducted in biological duplicate (±s.e.m and Mann-Whitney U tests are shown). (G) Trex1-mutant primary BMDMs from 3 different mice were transfected with 50 nM ASOs (or lipofectamine only, “Mock”) for 20 h, prior to RNA purification. Expression of the panel of 3 mouse IFN-driven genes was analysed by RT-qPCR. Expression of the indicated genes was reported to 18S expression and further normalised to the average of the “Mock” condition. Data shown represent the average of three mice conducted in biological duplicate (±s.e.m and Mann-Whitney U tests are shown). * P≤0.05, ** P≤0.01, *** P≤0.001, **** P≤0.0001, ns: non-significant.

FIG. 4. Sequence-dependent TLR9 inhibition. (A, C) HEK-TLR9 cells expressing a NF-κB-luciferase reporter were treated with 500 nM indicated ASOs for 30 min prior to stimulation or not (non-treated [NT]) with 200 nM ODN2006. NF-κB-luciferase levels were measured after overnight incubation. NF-κB-luciferase levels were normalised to the “ODN2006 only” condition, after background correction with NT condition. Data shown are averaged from two (A) or three (C) independent experiments in biological triplicate (±s.e.m and ordinary one-way ANOVA with Dunnett's multiple comparison tests to the “ODN2006 only” condition [A] or Mann-Whitney U tests [C] are shown). (B) ASO2 and ASO11 sequence mutants. ASO11Mutl and ASO11Mut2 contain the 3′ and 5′ end of ASO2, respectively. (D) HEK-TLR9 cells expressing a NF-κB-luciferase reporter were treated with 500 nM indicated ASOs for 45 min prior to stimulation or not with 200 nM ODN2006. NF-κB-luciferase levels were measured after overnight incubation. NF-κB-luciferase levels were normalised to “ODN2006 only” condition, after background correction with the NT condition. Stimulations and luciferase assays were carried out in two independent plates and the results presented on each axes (with a correlation r=0.7909, P≤0.0001)(averaged data are provided in Table 2). The 10 most potent ASOs (position reference in the plate is given as per Table 2) are highlighted on the plot. ASOs with ≤50% reduction of TLR9 activity at 500 nM are highlighted with blue shading. (E) Bottom: MEME pictogram of the relative frequency of bases constituting the TLR9 inhibitory motif Top: alignment of the sequences enriched with the motif identified FIG. 6D—position reference in the plate is given as per Table 2. (F) Alignment of the sequences enriched with ASO2 motif (Figure S6C). (G) The central DNA bases of the top and bottom 16 TLR9 inhibitors from the 80 ASOs screened (see Table 2) were analysed for base content. The violin plots show the distribution of the cumulative number of each central base for both ASO populations. Ordinary two-way ANOVA with Sidak's multiple comparison tests (between top and bottom populations) are shown. (H, J, L) HEK-TLR9 cells expressing a NF-κB-luciferase reporter were treated with 500 nM indicated ASOs for 45 min prior to stimulation or not (non-treated [NT]) with 200 nM ODN2006. NF-κB-luciferase levels were measured after overnight incubation. NF-κB-luciferase levels were normalised to “ODN2006 only” condition, after background correction with NT condition. Data shown are averaged from three independent experiments in biological triplicate (±s.e.m). One-way ANOVA with Dunnett's multiple comparison tests to the “AS02138” condition (H), or the “AS0108” condition (J), are shown. (L) One-way ANOVA with Tukey's multiple comparison tests relative to condition “661”, or otherwise indicated pairs of conditions are shown. (I, K) Sequence alignments showing the “CUU” motifs in families of closely related ASOs; the yellow region highlights the DNA moiety of the gapmers. (M) Correlation of TLR7 and TLR9 inhibition based on 80 ASOs (used at 100 nM for TLR7, and 500 nM for TLR9). Percentages of NF-κB-luciferase levels relative to the conditions “R848 without ASO” (for TLR7) or “ODN2006 without ASO” (for TLR9) are averaged from biological duplicate (averaged data are provided in Table 2) (with a correlation r-0.05256, P=0.6433). Selected ASOs are indicated. * P≤0.05, ** P≤0.01, **** P≤0.0001, ns: non-significant.

FIG. 5. The broad immunosuppressive effects of 2′OMe ASOs. (A) Bubble graph showing the relationship between cGAS, TLR9, TLR7 inhibition, and TLR8 potentiation (based on the data from Table 2 and (Alharbi et al., 2020)), for 80 ASOs. For TLR7 and TLR9, percentages of NF-κB-luciferase levels relative to the conditions “R848 without ASO” (for TLR7) or “ODN2006 without ASO” (TLR9) are shown (the size of the bubbles reflects TLR7 signalling strength). For TLR8, fold increases relative to the condition “R848 without ASO” are shown, using a 4 colour-scale. For cGAS, percentages of IP-10 production relative to the condition “ISD70 only” are shown. Selected ASOs are indicated—position reference in the plate is given as per Table 2. (B) HEK-TLR9 cells expressing a NF-κB-luciferase reporter were treated with 500 nM indicated ASOs for 30-45 min prior to stimulation or not (non-treated [NT]) with 200 nM ODN2006. NF-κB-luciferase levels were measured after overnight incubation. NF-κB-luciferase levels were normalised to “ODN2006 only” condition, after background correction with NT condition. Data shown are averaged from three independent experiments in biological triplicate (±s.e.m and one-way ANOVA with Tukey's multiple comparison tests relative to condition “ODN2006 only”, or otherwise indicated pairs of conditions are shown). (C) Correlation of TLR7 and cGAS inhibition based on 80 ASOs (used at 100 nM for TLR7 and cGAS). Percentages of NF-κB-luciferase levels relative to the conditions “R848 without ASO” (for TLR7) or percentages of IP-10 production relative to the condition “ISD70 only” are shown (with a significant correlation r=0.2780, P=0.0125). Selected ASOs are indicated—position reference in the plate is given as per Table 2. (D) HEK-TLR7 cells expressing a NF-κB-luciferase reporter were treated with indicated concentration of ASOs (500, 250, 125, 62.5, 31.25 nM) for 30-50 min prior to stimulation with 1 μg/ml R848. NF-κB-luciferase levels were measured after overnight incubation. NF-κB-luciferase levels were normalised to “R848 only” condition, after background correction with NT condition. Data shown are averaged from three independent experiments in biological triplicate (±s.e.m and ordinary two-way ANOVA with Sidak's multiple comparison tests relative to A151 are shown). (E) Correlation of TLR8 potentiation and cGAS inhibition based on 80 ASOs (used at 500 nM for TLR8 and 100 nM cGAS). Fold increases of NF-κB-luciferase levels relative to the conditions “R848 without ASO” (for TLR8) or percentages of IP-10 production relative to the condition “ISD70 only” are shown (with a significant correlation r=0.2879, P=0.0096). Selected ASOs are indicated—position reference in the plate is given as per Table 2. (F) HEK-TLR3 cells expressing a NF-κB-luciferase reporter were treated with indicated concentration of C2-Mutl (1000, 500, 250, 125, 62.5, nM) or A151 (753, 376.5, 188.25, 94.125 or 47.0625 nM) for 30-50 min prior to stimulation with 0.5 μg/ml pIC. NF-κB-luciferase levels were measured after overnight incubation. NF-κB-luciferase levels were normalised to “pIC only” condition, after background correction with NT condition. Data shown are averaged from three independent experiments in biological triplicate (±s.e.m). * P≤0.05, ** P≤0.01, *** P≤0.001, **** P≤0.0001, ns: non-significant.

FIG. 6. Multiple Em for motif Elicitation (MEME) motif discovery in ASOs. (A) The 2 most potent cGAS inhibitors in the 80 ASOs screen, including ASO2 (i.e. ASO2, C2 and E10) were analysed with EE. (B) The 10 most potent cGAS inhibitors in the 80 ASOs screen were analysed with EE (see Table 2). 5 sequences were identified with the motif shown (noting that C2 and F2 are closely related sequences, but the other ones were not). (C, D) The 10 most potent TLR9 inhibitors in the 80 ASOs screen were analysed with EE (see Table 2), along ASO2. Two motifs are shown here, including one coon with ASO2 (C), and an A-rich central motif (D). (E) EE analysis of 17 ASOs with less than 10% inhibition of cGAS sensing in the 80 ASOs screen (see Table 2). 8 ASOs shared the highlighted motif (noting that D10/A8 and A9/H9 are related sequences, respectively). (A-E) The figures are direct screenshots from the EE website. The ASO names are provided as their position in the plate (see Table 2).

FIG. 7. HT-29 were incubated with 500 nM ASO2-Cy3, prior to ISD70+lipofectamine transfection or not, for 4 h. The cells were subsequently washed with PBS prior to imaging by inverted fluorescent microscopy. The images shown are representative of two independent experiments. While cytosolic fluorescence in ASO2-Cy3 only treated cells clearly confirmed spontaneous uptake of the ASOs by the cells, the occurrence of bright fluorescent punctuates suggests an increased uptake of the labelled ASOs during the transfection of the lipofectamine-ISD complexes.

FIG. 8. THP-1 and HT-29 cells were incubated for 6 h with 100 nM or 187.5 nM C2-Mut1, respectively, prior to overnight transfection or not with ISD70. The next day, 1×resazurin solution was added to each well, and cell viability measured after 4-5 h at 37° C. Data were normalised to the NT condition (no ASO, no ISD70), after background correction with blank condition. Data shown are averaged from two independent experiments in biological triplicate (±s.e.m)

FIG. 9. (A) Primary FLS cells from 2 patients were cultivated for 15 days in the presence of indicated doses of naked ASOs, prior to being fixed and analysed for D-galactosidase staining. The numbers of β-galactosidase positive cells were normalised to the NT condition. Left panel: representative images of NT and 5 μM C2-Mutl conditions; 40-50% of the cells in NT conditions were positive for β-galactosidase staining. Data shown are averaged from three replicates for each condition (±s.e.m), for each independent donor. (B) Primary bone marrow-derived MSCs from two different patients were cultivated for 2 weeks in the presence of indicated doses of naked C2-Mutl, prior to being fixed and analysed for β-galactosidase staining. The numbers of f-galactosidase positive cells were normalised to the NT condition. Data shown are averaged from six replicates for each condition (±s.e.m), for each independent donor. (C) Primary FLS cells from 2 patients were cultivated for 7 days in the presence of 2.5 M naked ASOs, prior to being fixed and analysed for p3-galactosidase staining. The numbers of β-galactosidase positive cells were normalised to the NT condition. Data shown are averaged from at least five replicates for each condition (±s.e.m), for each independent donor. Ordinary two-way ANOVA with Sidak's multiple comparison tests relative to “C2-Mutl only” condition of each donor are shown.

FIG. 10. HEK-TLR3 cells expressing a NF-κB-luciferase reporter were treated with 500 nM (A) or 100 nM (B) indicated ASOs/ODNs for 20-50 min prior to stimulation or not (non-treated [NT]) with poly(I:C) (at 1 μg/ml [A] or 0.5 μg/ml [B]). NF-κB-luciferase levels were measured after overnight incubation and were normalised to “pIC only” condition, after background correction with NT condition. (A, B) Data are averaged from two independent experiments in biological triplicate (±s.e.m and one-way ANOVA with Dunnett's multiple comparison tests to the condition “pIC” are shown). ** P≤0.01, P≤0.0001. ns: non-significant.

FIG. 11. Inhibition of TLR9 sensing by ASO2 is preserved with decreasing amounts of ASO2. HEK-TLR9 cells expressing a NF-κB-luciferase reporter were treated with indicated amount of ASO2 (100-500 nM) for 50 min prior to stimulation or not (non-treated [NT]) with 200 nM ODN2006. NF-κB-luciferase levels were measured after overnight incubation. NF-κB-luciferase levels were normalised to “ODN2006 only” condition, after background correction with NT condition. Data shown are averaged from two independent experiments in biological triplicate (±s.e.m and one-way ANOVA with Dunnett's multiple comparison tests to the condition “NT” are shown). **** P≤0.0001. ns: non-significant.

FIG. 12. HEK-TLR7 cells expressing an NF-κB-luciferase reporter were treated with 500 nM indicated ASOs for 20 min prior to stimulation with 1 μg/ml R848. NF-κB-luciferase levels were measured after overnight incubation. Data are shown relative to the condition “R848 without ASO” are averaged from three (left panel) or two (right panel) independent experiments in biological triplicate (±s.e.m and ordinary one-way ANOVA with Dunnett's multiple comparison tests to the Mutl-dC condition [top] or NT condition [bottom]).

FIG. 13. THP-1 pre-treated for 45 min with 125 nM indicated ASOs were transfected with 2.5 μg/ml of ISD70 overnight and IP-10 levels in supernatants determined by ELISA. IP-10 levels were normalised to the “ISD70 only” condition, after background correction with the NT condition. Data shown are averaged from three independent experiments in biological triplicate (±s.e.m and ordinary one-way ANOVA with Dunnett's multiple comparison tests to the “ISD70 only” condition are shown).

FIG. 14: A, B) HEK-TLR7 cells expressing an NF-κB-luciferase reporter were pre-treated for −30 min with 100 nM (A) or 6 h with 400 nM (B) indicated 2′OMe ASOs, prior to R848 stimulation (1 μg/ml) overnight. All ASO conditions are with R848 co-stimulation. Data shown are averaged from a minimum of 2 (B) or 3 (A) independent experiments in biological triplicate, and reported to R848 only condition. SEM and One-way ANOVA with Dunnett's multiple comparisons to “R848 only” condition are shown. C) Primary BMDMs from 3 wildtype (WT) mice were pre-treated 30 min with 200 nM of indicated ASOs prior to treatment with 500 μM Guanosine overnight. The next day, supernatants were collected and analysed by TNFα ELISA. Data shown are averaged from 3 independent mice in biological triplicate. SEM and One-way ANOVA with Dunnett's multiple comparisons to “Guanosine only” condition are shown.

FIG. 15: Primary BMDMs from wildtype (WT) and Tlr7 Y264H mutant mice were pre-treated overnight with 200 nM of indicated ASOs prior to mRNA purification and RT-qPCR analyses. Gene expression was normalised to 18s levels, and further reported to the NT condition for each mouse (data are averaged from 2 wildtype and 3 Tlr7 Y264H mice, in biological duplicate). SEM and One-way ANOVA with Dunnett's multiple comparisons to Mut1-dC condition are shown.

FIG. 16: Left: sequence alignments of 2′MOE ASOs which displayed strongest TLR7 inhibition at 100 nM from the screen on HEK-TLR7 cells. The significantly enriched motif is highlighted in colour. Middle: MEME pictogram of the relative frequency of bases constituting the inhibitory motif Bottom: F5-Mut contains 3 base modifications (in red) targeted to the conserved motif (in blue shading), compared to F5. Right: HEK-TLR7 cells expressing an NF-κB-luciferase reporter were pre-treated for ˜30 min with 100 nM indicated 2′OMe ASOs, prior to R848 stimulation (1 μg/ml) overnight. All ASO conditions are with R848 co-stimulation. Data shown are averaged from a minimum of 3 independent experiments in biological triplicate and reported to R848 only condition. SEM and One-way ANOVA with Dunnett's multiple comparisons to “F5” condition are shown.

FIG. 17: THP-1, MG-63 and LL-171 cells were pre-treated for ˜30 min with indicated concentrations of ASOs, prior to overnight transfection with ISD. IP10 (for THP-1 and MG-63) and ISRE-Luc levels were measured and normalised to ISD only condition, after background correction. Data shown are averaged from 2 (LL171) or 3 independent experiments (THP-1 and MG-63) in biological triplicate. SEM, unpaired t-tests (THP-1) and ordinary two-way ANOVA with Dunnett's multiple comparisons to ASO847 (MG-63) are shown. All the conditions were stimulated with ISD except the NT condition.

FIG. 18: SV40T hTERT fibroblasts were transfected with 100 nM of indicated ASO overnight. RNA was purified the next day, and gene expression assessed by RTqPCR. Data obtained were normalised to 18S expression, and further reported to the values obtained for the Mock only condition (transfection reagent only). Data shown are averaged from 3 independent experiments in biological duplicate. SEM and One-way ANOVA with Dunnett's multiple comparisons to “847” condition [HPRT] or C2-Mutl [IFIT2] are shown.

FIG. 19: THP-1 cells were pre-treated for ˜30 min with 250 nM ASOs (except for C2-Mutl, used at 100 nM), prior to overnight transfection with ISD70. IP10 levels were measured and normalised to ISD70 only condition, after background correction. Data shown are averaged from 3 independent experiments (THP-1) in biological triplicate. SEM, and One-way ANOVA with Dunnett's multiple comparisons to “ISD70” only condition are shown. All the conditions were stimulated with ISD except the NT condition.

FIG. 20: THP-1 cells were pre-treated with 100 nM of ASOs overnight, prior to ISD70 stimulation for 7 h. Stimulations and ELISAs were carried out in two independent plates and the results presented on each axis (relative to ISD70 only controls). Selected wells which are presented in further analyses are highlighted. For both screens the plate A/B were significantly correlated, albeit there was more divergence with the 2MOE screen (correlation r=0.3760, P=0.0008) compared to the LNA screen (correlation r=0.7153, P≤0.0001).

FIG. 21: THP-1 cells were pre-treated for ˜30 min with 300 nM LNA or 200 nM MOE ASOs (except for C2-Mutl, used at 100 nM), prior to overnight transfection with ISD70. IP10 levels were measured and normalised to ISD70 only condition, after background correction. Data shown are averaged from 2 (LNA) or 3 (MOE) independent experiments in biological triplicate. SEM and One-way ANOVA with Dunnett's multiple comparisons to “ISD70” only condition are shown. All the conditions were stimulated with ISD70 except the NT condition.

FIG. 22: Mouse LL171 cells were pre-treated for ˜30 min with indicated concentrations of ASOs, prior to overnight transfection with ISD. ISRE-Luc levels were measured and normalised to ISD only condition, after background correction. Data shown are averaged from 2 independent experiments in biological triplicate. SEM and ordinary one-way ANOVA with Dunnett's multiple comparisons to ISD only conditions are shown. All the conditions were stimulated with ISD except the NT condition.

FIG. 23: THP-1 and LL-171 cells were pre-treated for −30 min with 200 (LL171 and THP-1 with 2MOE) or 300 (THP-1 with LNA) nM ASOs, prior to overnight transfection with ISD. IP10 (for THP-1) and ISRE-Luc levels (for LL171) were measured and normalised to ISD only condition, after background correction. Data shown are averaged from 3 independent experiments in biological triplicate. SEM, unpaired Mann-Whitney (LL171) and ordinary one-way ANOVA (THP-1) with Dunnett's multiple comparisons to ISD only are shown. All the conditions were stimulated with ISD except the NT condition.

FIG. 24: MG-63 and LL-171 cells were pre-treated for ˜30 min with indicated concentrations of ASOs, prior to overnight transfection with ISD. IP10 (for MG-63) and ISRE-Luc levels (for LL171) were measured and normalised to ISD only condition, after background correction. Data shown are averaged from 3 independent experiments in biological triplicate (+/−SEM).

FIG. 25: MG-63 were pre-treated for −30 min with 1 mM indicated concentrations of ASOs (except for B3 and HPRT847Mut, used at 500 nM), prior to overnight transfection with ISD, stimulation with 5 mg/ml pIC or 100 nM GSK (Valentin et al., 2021). IP10 levels were measured and normalised to GSK only condition, after background correction. Data shown are from a single experiment in biological triplicate (+/−SEM).

FIG. 26: iBMDMs were pre-treated for −30 min with 500 nM indicated concentrations of ASOs prior to overnight stimulation with ISD or lipofectamine only (“Mock” conditions). IP10 levels were measured and normalised to ISD only condition, after background correction. Data shown are averaged from 3 independent experiments in biological triplicate. SEM and One-way ANOVA with Dunnett's multiple comparisons to “ISD” only condition are shown. All the conditions were stimulated with ISD except the NT and Mock conditions.

FIG. 27: THP-1 cells were pre-treated for ˜30 min with indicated concentration of ASOs prior to overnight transfection with ISD70. IP10 levels were measured and normalised to ISD70 only condition, after background correction. Data shown are averaged from 3 independent experiments in biological triplicate. SEM and non-linear regressions are shown. All the conditions were stimulated with ISD70.

FIG. 28: Recombinant cGAS protein was incubated in vitro with 2.3 mM ISD70 with or without (NT) 2 μM ASO for 40 min. The reaction was stopped with EDTA and cGAMP levels were analysed by specific ELISA. Data is from a single experiment and points are from technical replicates.

FIG. 29: HEK-TLR9 cells expressing a NF-κB-luciferase reporter were treated with 100 or 500 nM ASOs for 45 min prior to stimulation or not with 200 nM ODN2006. NF-κB-luciferase levels were measured after overnight incubation. NF-κB-luciferase levels were normalised to the ‘ODN2006 only’ condition, after background correction with the NT condition. Stimulations with 100 nM and 500 nM ASO were performed in independent experiments (data shown for each concentration is from a single experiment in biological duplicate). MOE D10 (in pink) and D8 LNA (in green) are both targeting the same region of MB21D1, and thus are similar to the ASO2 previously studied as the best TLR9 2′OMe ASO (Valentin et al. 2021).

FIG. 30: HEK-TLR9 cells expressing a NF-κB-luciferase reporter were treated with 100 nM indicated ASOs for 45 min prior to stimulation or not with 200 nM ODN2006 (CpG). NF-κB-luciferase levels were measured after overnight incubation. NF-κB-luciferase levels were normalised to the ‘CpG only’ condition, after background correction with the NT. Data shown are averaged from 3 independent experiments in biological triplicate. SEM and One-way ANOVA with Dunnett's multiple comparisons to “CpG” only condition are shown. All the conditions were stimulated with ODN2006 except the NT conditions.

FIG. 31: HEK-TLR9 cells expressing a NF-κB-luciferase reporter were treated with 100 nM indicated ASOs for 45 min prior to stimulation or not with 200 nM ODN2006 (CpG). NF-κB-luciferase levels were measured after overnight incubation. NF-κB-luciferase levels were normalised to the ‘CpG only’ condition, after background correction with the NT. Data shown are averaged from 3 independent experiments in biological triplicate. SEM and One-way ANOVA with Dunnett's multiple comparisons to “CpG” only condition are shown. All the conditions were stimulated with ODN2006 except the NT conditions.

FIG. 32: Primary BMDMs from wildtype (WT) mice were pre-treated with 100 nM of indicated ASOs for 1h prior to transfection with 250 nM of B-406-AS RNA overnight. The next day, supernatants were collected and analysed by TNFα ELISA. Data shown are averaged from 2 independent mice in biological triplicate. SEM and One-way ANOVA with Dunnett's multiple comparisons to “B-406-AS+dC20” condition are shown.

FIG. 33: HEK-TLR8 cells expressing an NF-κB-luciferase reporter were pre-treated ˜30 min with 500 nM ASOs, prior to R848 stimulation overnight. Data shown are averaged from 2 independent experiments in biological triplicate. The NF-κB-luciferase values are reported to the R848 condition. All ASO conditions are with R848 co-stimulation. SEM and One-way ANOVA with Dunnett's multiple comparisons to R848 only condition are shown.

FIG. 34: THP-1 cells were pre-treated overnight with 1 mM ASOs, prior to R848 stimulation for 7h. Data shown are averaged from 3 independent experiments in biological triplicate. All ASO conditions are with R848 co-stimulation. SEM and One-way ANOVA with Dunnett's multiple comparisons to R848 only condition are shown.

FIG. 35: THP-1 cells were pre-treated overnight with indicated concentrations of ASOs, prior to R848 stimulation for 7h. Data shown are averaged from 3 independent experiments in biological triplicate. All ASO conditions are with R848 co-stimulation. SEM and two-way ANOVA with Tukey's multiple comparisons to 660-3b condition are shown.

FIG. 36: A, B, C) HEK-TLR8 cells expressing an NF-κB-luciferase reporter were pre-treated 40 min with 1 mM (B), or 5 mM (A and C) oligos, prior to Motolimod stimulation (400 nM [B] or 600 nM [A and C] overnight). Data shown are averaged from 2 (B) independent or a single experiment (A and C), completed with 3 biological triplicate. The NF-κB-luciferase values are reported to the Motolimod condition. D) THP-1 cells were pre-treated overnight with 1 mM 3-mer oligos, prior to R848 stimulation (1 mg/ml) for 7h. IP-10 levels were measured by ELISA. Data shown are averaged from a single experiment completed with 3 biological triplicate, reported to the R848 only condition. A-E) All oligo conditions are with Motolimod or R848 co-stimulation.

FIG. 37: A, B and C) HEK-TLR7 cells expressing an NF-κB-luciferase reporter were pre-treated for ˜30 min with 400 nM (A, C) or indicated dose of 2′OMe 3-mer oligos, prior to R848 stimulation (1 μg/ml [A, B) or indicated dose [C]) overnight. All oligo conditions are with R848 co-stimulation. Data shown are averaged from a minimum of 2 (A, C) or 4 (B) independent experiments in biological triplicate, and reported to R848 only condition (A, B) or NT (C). SEM and One-way ANOVA with Dunnett's multiple comparisons to “R848 only” condition (A) or two-way ANOVA (B, C) are shown.

FIG. 38: HEK-TLR7 cells expressing an NF-κB-luciferase reporter were pre-treated for ˜30 min with 400 nM or 2 μM or indicated dose of 2′OMe 3-mer oligos, prior to overnight R848 stimulation (1 μg/ml). All oligo conditions are with R848 co-stimulation. Data shown are averaged from 2 independent experiments in biological triplicate, and reported to R848 only condition after background correction to NT.

FIG. 39: THP-1 cells were pre-treated with indicated dose (A) or 250 nM (B) ASOs for 30 min, prior to ISD70 stimulation overnight. IP10 levels were measured and normalised to ISD70 only condition, after background correction. Data shown are averaged (A) or representative (B) from 2 independent experiments in biological triplicate. SEM are shown. All the conditions were stimulated with ISD70 except the NT condition.

FIG. 40: Immortalised mouse bone marrow derived macrophages were pre-treated for ˜30 min with 250 nM of 2′OMe ASOs, prior to ODN stimulation (500 nM) overnight. All oligo conditions are with ODN co-stimulation. Data shown are averaged from 2 independent experiments in biological triplicate, and reported to ODN only condition. SEM and One-way ANOVA with Dunnett's multiple comparisons to “C2Mut1vl only” are shown.

FIG. 41: HEK-TLR9 cells expressing a NF-κB-luciferase reporter were treated with 2 mM ASOs for 45 min prior to stimulation or not with 200 nM ODN2006. NF-κB-luciferase levels were measured after overnight incubation. NF-κB-luciferase levels were normalised to the ‘ODN2006 only’ condition, after background correction with the NT condition. ASO2 was used as a positive control. Data shown are averaged from 1 experiment with 3 biological replicate.

FIG. 42: HEK-TLR3 cells expressing a NF-κB-luciferase reporter were treated with 2 mM 3-mer 2′OMe for 45 min prior to stimulation or not with 500 ng/ml polyI:C. NF-κB-luciferase levels were measured after overnight incubation. NF-κB-luciferase levels were normalised to the ‘polyL:C only’ condition, after background correction with the NT condition. C2-Mutl was used as a positive control. Data shown are averaged from 1 experiment with 3 biological replicate.

FIG. 43: HEK-TLR3 cells expressing a NF-κB-luciferase reporter were treated with 2 mM 3-mers DNA PS for 45 min prior to stimulation or not with 500 ng/ml polyL:C. NF-κB-luciferase levels were measured after overnight incubation. NF-κB-luciferase levels were normalised to the ‘polyL:C only’ condition, after background correction with the NT condition. C2-Mutl was used as a positive control. Data shown are averaged from 1 experiment with 3 biological replicate.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in oligonucleotide design, molecular genetics, antisense oligonucleotides, gene silencing, gene expression and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sabrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. Glover and B. D. Haes (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

By “consisting essentially of” in the context of an oligonucleotide sequence is meant the recited oligonucleotide sequence together with an additional one, two or three nucleic acids at the 5′ or 3′ end thereof.

As used herein, the phrase “inhibits cGAS activity” or variations thereof means that after administration of an oligonucleotide of the invention to an animal, the animal is not able to elicit a cGAS based immune response or is only able to elicit a reduced or partial cGAS based immune response, such as to a pathogen or a damaged endogenous nucleic acid. In an embodiment, the cGAS based immune response is less than about 95%, 90%, 80%, 70%, 60%, 50%, 40%, 30%, 20%, 1%, 50%, 2% or 1% of the response in the absence of the oligonucleotide.

Similarly, the phrase “increasing the cGAS inhibitory activity of an oligonucleotide” or the like means that after being modified in accordance with the invention, an animal administered with the modified oligonucleotide is not able to mount a cGAS based immune response or only able to mount a weaker or partial cGAS based immune response, such as to a pathogen or a damaged endogenous nucleic acid, when compared to the starting (unmodified) oligonucleotide.

As used herein, the phrase “does not inhibit cGAS activity” or variations thereof means that after administration of an oligonucleotide of the invention to an animal, the animal is not able to elicit a cGAS based immune response, such as to a pathogen or a damaged endogenous nucleic acid. In an embodiment, the cGAS based immune response is at least 10%, 20%, 30%, 40%, 50%, 60%, 70%, 80%, 85%, 90%, 95%, 97%, 99% or 100% of the response in the absence of the oligonucleotide.

Similarly, the phrase “reducing the cGAS inhibitory activity of an oligonucleotide” or the like means that after being modified in accordance with the invention, an animal administered with the modified oligonucleotide is able to mount a stronger cGAS based immune response, such as to a pathogen or a damaged endogenous nucleic acid, when compared to the starting (unmodified) oligonucleotide.

As used herein, the phrase “inhibits TLR9 activity” or variations thereof means that after administration of an oligonucleotide of the invention to an animal, the animal is not able to elicit a TLR9 based immune response or is only able to elicit a reduced or partial TLR9 based immune response, such as to a pathogen or a damaged endogenous nucleic acid. In an embodiment, the TLR9 based immune response is less than about 95%, 90%, 80%, 70%, 60%, 50%, 40%, 30%, 20%, 1%, 50%, 2% or 1% of the response in the absence of the oligonucleotide.

Similarly, the phrase “increasing the TLR9 inhibitory activity of an oligonucleotide” or the like means that after being modified in accordance with the invention, an animal administered with the modified oligonucleotide is not able to mount a TLR9 based immune response or only able to mount a weaker or partial TLR9 based immune response, such as to a pathogen or a damaged endogenous nucleic acid, when compared to the starting (unmodified) oligonucleotide.

As used herein, the phrase “does not inhibit TLR9 activity” or variations thereof means that after administration of an oligonucleotide of the invention to an animal, the animal is not able to elicit a TLR9 based immune response, such as to a pathogen or a damaged endogenous nucleic acid. In an embodiment, the TLR9 based immune response is at least 10%, 20%, 30%, 40%, 50%, 60%, 70%, 80%, 85%, 90%, 95%, 97%, 99% or 100% of the response in the absence of the oligonucleotide.

As used herein, the phrase “inhibits TLR7 activity” or variations thereof means that after administration of an oligonucleotide of the invention to an animal, the animal is not able to elicit a TLR7 based immune response or is only able to elicit a reduced or partial TLR7 based immune response, such as to a pathogen or a damaged endogenous nucleic acid. In an embodiment, the TLR7 based immune response is less than about 95%, 90%, 80%, 70%, 60%, 50%, 40%, 30%, 20%, 10%, 5%, 2% or 1% of the response in the absence of the oligonucleotide.

As used herein, the phrase “does not inhibit TLR7 activity” or variations thereof means that after administration of an oligonucleotide of the invention to an animal, the animal is not able to elicit a TLR7 based immune response, such as to a pathogen or a damaged endogenous nucleic acid. In an embodiment, the TLR7 based immune response is at least 10%, 20%, 30%, 40%, 50%, 60%, 70%, 80%, 85%, 90%, 95%, 97%, 99% or 100% of the response in the absence of the oligonucleotide.

As used herein, the phrase “inhibits TLR8 activity” or variations thereof means that after administration of an oligonucleotide of the invention to an animal, the animal is not able to elicit a TLR8 based immune response or is only able to elicit a reduced or partial TLR8 based immune response, such as to a pathogen or a damaged endogenous nucleic acid. In an embodiment, the TLR8 based immune response is less than about 95%, 90%, 80%, 70%, 60%, 50%, 40%, 30%, 20%, 10%, 5%, 2% or 1% of the response in the absence of the oligonucleotide.

As used herein, the phrase “increases or potentiates TLR8 activity” or variations thereof means that after administration of an oligonucleotide of the invention to an animal, the animal is able to elicit a TLR8 based immune response or is only able to elicit an increases or elevated TLR8 based immune response, such as to a pathogen or a damaged endogenous nucleic acid. In an embodiment, the TLR8 based immune response is more than about 95%, 90%, 80%, 70%, 60%, 50%, 40%, 30%, 20%, 10%, 5%, 2% or 1% of the response in the absence of the oligonucleotide.

As used herein, the phrase “inhibits TLR3 activity” or variations thereof means that after administration of an oligonucleotide of the invention to an animal, the animal is not able to elicit a TLR3 based immune response or is only able to elicit a reduced or partial TLR3 based immune response, such as to a pathogen or a damaged endogenous nucleic acid. In an embodiment, the TLR3 based immune response is less than about 95%, 90%, 80%, 70%, 60%, 50%, 40%, 30%, 20%, 10%, 5%, 2% or 1% of the response in the absence of the oligonucleotide.

As used herein, the phrase “does not inhibit TLR3 activity” or variations thereof means that after administration of an oligonucleotide of the invention to an animal, the animal is not able to elicit a TLR3 based immune response, such as to a pathogen or a damaged endogenous nucleic acid. In an embodiment, the TLR3 based immune response is at least 10%, 20%, 30%, 40%, 50%, 60%, 70%, 80%, 85%, 90%, 95%, 97%, 99% or 100% of the response in the absence of the oligonucleotide.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

As used herein, the terms “treating”, “treat” or “treatment” include administering a therapeutically effective amount of a compound(s) described herein sufficient to reduce or eliminate at least one symptom of a disease, disorder or condition.

As used herein, the terms “preventing”, “prevent” or “prevention” include administering a therapeutically effective amount of a compound(s) described herein sufficient to stop or hinder the development of at least one symptom of a disease, disorder or condition.

The terms “therapeutically effective amount” and “effective amount” describe a quantity of a specified agent, such as an oligonucleotide of the invention, sufficient to achieve a desired effect in a subject or cell being treated or contacted with that agent. For example, this can be the amount of a composition comprising one or more agents that inhibit the activity of one or more nucleic acid sensors (e.g., cGAS, TLR3, TLR7, TLR8 or TLR9) described herein, necessary to reduce, alleviate and/or prevent a disease, disorder or condition. In some embodiments, a “therapeutically effective amount” is sufficient to reduce or eliminate a symptom of a disease, disorder or condition. In other embodiments, a “therapeutically effective amount” or “effective amount” is an amount sufficient to achieve a desired biological effect, for example, an amount that is effective to decrease or prevent a senescence-associated disease, disorder or condition or inhibit or prevent senescence in a cell.

Ideally, a therapeutically effective amount of an agent is an amount sufficient to induce the desired result without causing a substantial cytotoxic effect in the subject. The effective amount of an agent useful for reducing, alleviating and/or preventing a disease, disorder or condition will be dependent on the subject being treated, the type and severity of any associated symptoms and the manner of administration of the therapeutic composition.

### Oligonucleotides

In the context of this invention, the term “oligonucleotide” refers to an oligomer or polymer of ribonucleic acid (RNA) and/or deoxyribonucleic acid (DNA), wherein the polymer or oligomer of nucleotide monomers contains any combination of nucleobases (referred to in the art and herein as simply as “base”), modified nucleobases, sugars, modified sugars, phosphate bridges, or modified phosphorus atom bridges (also referred to herein as “internucleotidic linkage”).

Oligonucleotides can be single-stranded or double-stranded or a combination thereof A single-stranded oligonucleotide can have double-stranded regions and a double-stranded oligonucleotide can have single-stranded regions (such as a microRNA or shRNA).

Oligonucleotides generally refer to relatively short sequences of nucleotides, typically with twenty or fewer bases (or nucleotide units), but can also be significantly longer, such as up to about 160 to about 200 nucleotides. By way of example, the oligonucleotide provided herein is at least about 3, 5, 10, 15, 20, 25, 30, 35, 40, 45, 50, 60, 70, 80, 90, 100 nucleotides, or any range therein, in length. More particularly, the oligonucleotide is suitably about 3 to about 75 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 nucleotides, or any range therein) in length. In particular embodiments, the oligonucleotide is about 3 to about 5 nucleotides in length, as shown above, and including but not limited to (e.g., 5′-GGUAU-3′ (SEQ ID NO: 56), 5′-GGUA-3′ (SEQ ID NO: 57), 5′-GUAU-3′ (SEQ ID NO: 58), 5′-GGU-3′ (SEQ ID NO: 59) or 5′-GUA-3′ (SEQ ID NO: 60)). In other embodiments, the oligonucleotide is about to about 25 nucleotides in length. In some embodiments, the oligonucleotide is about nucleotides in length.

“Gapmer” refers to an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

As used herein, a “target” such as a “target gene” or “target polynucleotide” refers to a molecule upon which an oligonucleotide of the invention directly or indirectly exerts its effects. Typically, the oligonucleotide of the invention or portion thereof and the target, or a product of the target such as mRNA encoded by a gene, or portion thereof, are able to hybridize under physiological conditions.

As used herein, the phrase “reduces expression of the target gene” or the like refers to an oligonucleotide of the invention reducing the ability of a gene to exert is biological effect. This can be directly or indirectly achieved by reduction in the amount of RNA encoded by the gene and/or reduction of the amount of protein translated from an RNA.

Typically, an oligonucleotide of the invention will be synthesized in vitro. However, in some instances where modified bases and backbone are not required they can be expressed in vitro or in vivo in a suitable system such as by a recombinant virus or cell.

An oligonucleotide of the invention may be conjugated to one or more moieties or groups which enhance the activity, cellular distribution or cellular uptake of the oligonucleotide. These moieties or groups may be covalently bound to functional groups such as primary or secondary hydroxyl groups. Exemplary moieties or groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins and dyes.

As used herein, a “synthetic oligonucleotide sequence” refers to an oligonucleotide sequence which lacks a corresponding sequence that occurs naturally. By way of example, a synthetic oligonucleotide sequence is not complementary to a specific RNA molecule, such as one encoding an endogenous polypeptide. As such, the synthetic oligonucleotide sequence is suitably not capable of interfering with a post-transcriptional event, such as RNA translation.

As used herein, an oligonucleotide “variant” shares a definable nucleotide sequence relationship with a reference nucleic acid sequence. The reference nucleic acid sequence may be one of those provided in Tables 1 and 2, for example. The “variant” oligonucleotide may have one or a plurality of nucleic acids of the reference nucleic acid sequence deleted or substituted by different nucleic acids. Preferably, oligonucleotide variants share at least 70% or 75%, preferably at least 80% or 85% or more preferably at least 90%, 91%, 92%, 93%, 94%, 95%, 96%, 97%, 98% or 99% sequence identity with a reference nucleic acid sequence.

### Modified Bases

Olfigonucleotides of the invention may have nucleobase (“base”) modifications or substitutions.

Examples include oligonucleotides comprising one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In one embodiment, the oligonucleotide comprises one of the following at the 2′ position: O[(CH2)nO]mCH3, O(CH2)nOCH3, O(CH2)nNH2, O(CH2)nCH3, O(CH2)nONH2, and O(CH2)nON[(CH2)nCH3]2, where n and m are from 1 to about 10.

Further examples include of modified oligonucleotides include oligonucleotides comprising one of the following at the 2′ position: C1 to C10 lower alkyl, substituted lower alkyl, alkenyl, alkynyl, alkaryl, aralkyl, O-alkaryl or O-aralkyl, SH, SCH3, OCN, C1, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

In one embodiment, the modification includes 2′-methoxyethoxy (2′-O-CH2CH2OCH3 (also known as 2′-O-(2-methoxyethyl) or 2′-MOE) (Martin et al., 1995), that is, an alkoxyalkoxy group. In a further embodiment, the modification includes 2′-dimethylaminooxyethoxy, that is, a O(CH2)20N(CH3)2 group (also known as 2′-DMAOE), or 2′-dimethylaminoethoxyethoxy (also known in the art as 2′-O-dimethyl-amino-ethoxy-ethyl or 2′-DMAEOE), that is, 2′-O-CH2-O-CH2-N(CH3)2.

Other modifications include 2′-methoxy (2′-O-CH3), 2′-aminopropoxy (2′-OCH2CH2CH2NH2), 2′-allyl (2′-CH2-CH═CH2), 2′-O-allyl (2′-O-CH2-CH═CH2) and 2′-fluoro (2′-F). The 2-modification may be in the arabino (up) position or ribo (down) position. In one embodiment a 2′-arabino modification is 2′-F.

Similar modifications may also be made at other positions on the oligonucleotide, particularly the 3′ position of the sugar on the 3′ terminal nucleotide or in 2′-5′ linked oligonucleotides and the 5′ position of the 5′ terminal nucleotide.

Oligonucleotides may also have sugar mimetics, such as cyclobutyl moieties in place of the pentofuranosyl sugar.

Representative United States patents that teach the preparation of such modified sugar structures include, but are not limited to, U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,658,873, 5,670,633, 5,792,747, and 5,700,920.

A further modification of the sugar includes Locked Nucleic Acids (LNAs) in which the 2′-hydroxyl group is linked to the 3′ or 4′ carbon atom of the sugar ring, thereby forming a bicyclic sugar moiety. In one embodiment, the linkage is a methylene (—CH2-)n group bridging the 2′ oxygen atom and the 4′ carbon atom, wherein n is 1 or 2. LNAs and preparation thereof are described in WO 98/39352 and WO 99/14226.

Modified nucleobases include other synthetic and natural nucleobases such as, for example, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—CC—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine, m¹A(1-methyladenosine); m²A(2-methyladenosine); Am (2′-O-methyladenosine); ms²m6A(2-methylthio-N⁶-methyladenosine); i⁶A (N⁶-isopentenyladenosine); ms²t⁶A(2-methylthio-N⁶isopentenyladenosine); io⁶A(N⁶-(cis-hydroxyisopentenyl)adenosine); ms²iO⁶A(2-methylthio-N⁶-(cis-hydroxyisopentenyl)adenosine); g⁶A(N6-glycinylcarbamoyladenosine); t⁶A(N⁶-threonylcarbamoyladenosine); ms²t6A(2-methylthio-N⁶-threonyl carbamoyladenosine); m⁶t6A(N⁶-methyl-N⁶-threonylcarbamoyladenosine); hn⁶A(N⁶-hydroxynorvalylcarbamoyladenosine); ms²hn⁶A(2-methylthio-N⁶-hydroxynorvalyl carbamoyladenosine); Ar(p)(2′-O-ribosyladenosine(phosphate)); I (inosine); m¹I(1-methylinosine); m¹Im(1,2′-O-dimethylinosine); m³C(3-methylcytidine); Cm(2′-O-methylcytidine); s²C(2-thiocytidine); ac⁴C(N⁴-acetylcytidine); f⁵C (5-formylcytidine); m⁵Cm(5,2′-O-dimethylcytidine); ac⁴Cm(N⁴-acetyl-2′-O-methylcytidine); k²C(lysidine); m¹G(1-methylguanosine); m²G(N²-methylguanosine); m⁷G(7-methylguanosine); Gm(2′-O-methylguanosine); m²2G(N2,N2-dimethylguanosine); m²Gm(N²,2′-O-dimethylguanosine); m²2Gm(N²,N²,2′-O-trimethylguanosine); Gr(p)(2′-O-ribosylguanosine (phosphate)); yW (wybutosine); o²yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQ₁ (7-aminomethyl-7-deazaguanosine); G⁺(archaeosine); D (dihydrouridine); m⁵Um(5,2′-O-dimethyluridine); s⁴U(4-thiouridine); m⁵s²U(5-methyl-2-thiouridine); s²Um(2-thio-2′-O-methyluridine); acp³U(3-(3-amino-3-carboxypropyl)uridine); ho⁵U(5-hydroxyuridine); mo⁵U(5-methoxyuridine); cmo⁵U(uridine 5-oxyacetic acid); mcmo⁵U (uridine 5-oxyacetic acid methyl ester); chm⁵U(5-(carboxyhydroxymethyl)uridine)); mchm⁵U(5-(carboxyhydroxymethyl)uridine methyl ester); mcm⁵U(5-methoxycarbonylmethyluridine); mcm⁵Um(5-methoxycarbonylmethyl-2′-O-methyluridine); mcm⁵s²U(5-methoxycarbonylmethyl-2-thiouridine); nm⁵s²U(5-aminomethyl-2-thiouridine); mnm⁵U(5-methylaminomethyluridine); mnm⁵s²U(5-methylaminomethyl-2-thiouridine); mnm⁵se²U(5-methylaminomethyl-2-selenouridine); ncm⁵U(5-carbamoylmethyluridine); nCm⁵Um(5-carbamoylmethyl-2′-O-methyluridine); cmnm⁵U(5-carboxymethylaminomethyluridine); cmnm⁵Um(5-carboxymethylaminomethyl-2′-O-methyluridine); cmnm⁵s²U(5-carboxymethylaminomethyl-2-thiouridine); m⁶₂A(N⁶,N⁶-dimethyladenosine); Im(2′-O-methylinosine); m⁴C(N4-methylcytidine); m⁴Cm(N⁴,2′-O-dimethylcytidine); hm⁵C(5-hydroxymethylcytidine); m³U(3-methyluridine); cm⁵U(5-carboxymethyluridine); m⁶Am(N⁶,2′-O-dimethyladenosine); m⁶₂Am (N⁶,N⁶,O-2′-trimethyladenosine); m²,7G(N2,7-dimethylguanosine); m^{2,2,7}G(N2,N2,7-trimethylguanosine); m³Um(3,2′-O-dimethyluridine); m⁵D(5-methyldihydrouridine); f⁵Cm (5-formyl-2′-O-methylcytidine); m¹Gm (1,2′-O-dimethylguanosine); m¹Am(1,2′-O-dimethyladenosine); τm⁵U(5-taurinomethyluridine); τm⁵s²U(5-taurinomethyl-2-thiouridine)); imG-14 (4-demethylwyosine); imG2(isowyosine); or ac⁶A(N⁶-acetyladenosine).

Further modified nucleobases include tricyclic pyrimidines, such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as, for example, a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one).

Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in U.S. Pat. No. 3,687,808, those disclosed in J. I. Kroschwitz (editor), The Concise Encyclopedia of Polymer Science and Engineering, pages 858-859, John Wiley and Sons (1990), those disclosed by Englisch et al. (1991), and those disclosed by Y. S. Sanghvi, Chapter 15: Antisense Research and Applications, pages 289-302, S. T. Crooke, B. Lebleu (editors), CRC Press, 1993.

Certain of these nucleobases are particularly useful for increasing the binding affinity of the oligonucleotide. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2 oC. In one embodiment, these nucleobase substitutions are combined with 2′-O-methoxyethyl sugar modifications.

Representative United States patents that teach the preparation of certain of the above noted modified nucleobases as well as other modified nucleobases include, but are not limited to, U.S. Pat. Nos. 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, U.S. Pat. Nos. 5,614,617, 5,645,985, 5,830,653, 5,763,588, 6,005,096, 5,681,941 and 5,750,692.

Unless stated to the contrary, reference to an A, T, G, U or C can either mean a naturally occurring base or a modified version thereof.

### Backbones

Oligonucleotides of the present disclosure include those having modified backbones or non-natural internucleoside linkages. Oligonucleotides having modified backbones include those that retain a phosphorus atom in the backbone and those that do not have a phosphorus atom in the backbone.

Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more intemucleotide linkages is a 3′ to 3′, 5′ to 5′ or 2′ to 2′ linkage. Oligonucleotides having inverted polarity comprise a single 3′ to 3′ linkage at the 3′-most intemucleotide linkage, that is, a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808, 4,469,863, 4,476,301, 5,023,243, 5,177,196, 5,188,897, 5,264,423, 5,276,019, 5,278,302, 5,286,717, 5,321,131, 5,399,676, 5,405,939, 5,453,496, 5,455,233, 5,466,677, 5,476,925, 5,519,126, 5,536,821, 5,541,306, 5,550,111, 5,563,253, 5,571,799, 5,587,361, 5,194,599, 5,565,555, 5,527,899, 5,721,218, 5,672,697 and 5,625,050.

Modified oligonucleotide backbones that do not include a phosphorus atom therein include, for example, backbones formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

Representative United States patents that teach the preparation of the above oligonucleotides include, but are not limited to, U.S. Pat. Nos. 5,034,506, 5,166,315, 5,185,444, 5,214,134, 5,216,141, 5,235,033, 5,264,562, 5,264,564, 5,405,938, 5,434,257, 5,466,677, 5,470,967, 5,489,677, 5,541,307, 5,561,225, 5,596,086, 5,602,240, 5,610,289, 5,602,240, 5,608,046, 5,610,289, 5,618,704, 5,623,070, 5,663,312, 5,633,360, 5,677,437, 5,792,608, 5,646,269 and 5,677,439.

### Antisense Oligonucleotides

The term “antisense oligonucleotide” shall be taken to mean an oligonucleotide that is complementary to at least a portion of a specific mRNA molecule, such as encoding an endogenous polypeptide and capable of interfering with a post-transcriptional event such as mRNA translation. The use of antisense methods is well known in the art (see for example, G. Hartmann and S. Endres, Manual of Antisense Methodology, Kluwer (1999)).

In one embodiment, the antisense oligonucleotide hybridises under physiological conditions, that is, the antisense oligonucleotide (which is fully or partially single stranded) is at least capable of forming a double stranded polynucleotide with mRNA, such as encoding an endogenous polypeptide, under normal conditions in a cell.

Antisense oligonucleotides may include sequences that correspond to the structural genes or for sequences that effect control over the gene expression or splicing event. For example, the antisense sequence may correspond to the targeted coding region of endogenous gene, or the 5-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The antisense oligonucleotide may be complementary to the entire gene transcript, or part thereof. The degree of identity of the antisense sequence to the targeted transcript should be at least 90% and more preferably 95-100%. The antisense RNA or DNA molecule may of course comprise unrelated sequences which may function to stabilize the molecule such as described herein.

### Gene Silencing

Oligonucleotide molecules, particularly RNA, may be employed to regulate gene expression. The terms “RNA interference”, “RNAi” or “gene silencing” refer generally to a process in which a dsRNA molecule reduces the expression of a nucleic acid sequence with which the double-stranded RNA molecule shares substantial or total homology. However, it has been shown that RNA interference can be achieved using non-RNA double stranded molecules (see, for example, US 20070004667).

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a polynucleotide which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof The siRNA can be assembled from two separate oligonucleotides, where one strand is the sense strand and the other is the antisense strand, wherein the antisense and sense strands are self-complementary. The two strands can be of different length.

As used herein, the term siRNA is meant to be equivalent to other terms used to describe polynucleotides that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules can be used to epigenetically silence genes at both the post-transcriptional level and the pre-transcriptional level. In a non-limiting example, epigenetic regulation of gene expression by siRNA molecules can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity. An Example of a sequence of a single-stranded loop includes: 5′ UUCAAGAGA 3′.

Included shRNAs are dual or bi-finger and multi-finger hairpin dsRNAs, in which the RNA molecule comprises two or more of such stem-loop structures separated by single-stranded spacer regions.

### Design and Testing of Candidate Oligonucleotides

As the skilled person is aware, in addition to design elements of the invention, there are many known factors to be considered when producing an oligonucleotide. The specifics depend on the purpose of the oligonucleotide but include features such as strength and stability of the oligonucleotide-target nucleic acid interaction, such as the mRNA secondary structure, thermodynamic stability, the position of the hybridization site, and/or functional motifs.

Some methods the invention involve scanning a target polynucleotide, or complement thereof, for specific features. This can be done by eye or using computer programs known in the art. Software programs which can be used to design, analyse and predict functional properties of antisense oligonucleotides include Mfold, Sfold, NUPACK, Nanofolder, Hyperfold, and/or RNA designer. Software programs which can be used to design, analyse and predict functional properties of oligonucleotides for gene silencing include dsCheck, E-RNAi and/or siRNA-Finder.

In one embodiment, available software is used to select potentially useful oligonucleotides, and then these are scanned for desired features as described herein. Alternatively, software could readily be developed to scan a target polynucleotide, or complement thereof, for desired features as described herein.

Once synthesized, candidate oligonucleotides can be tested for their desired activity using standard procedures in the art. This may involve administering the candidate to cells in vitro expressing the gene of interest and analysing the amount of gene product such as RNA and/or protein. In another example, the candidate is administered to an animal, and the animal screened for the amount of target RNA and/or protein and/or using a functional assay. In another embodiment, the oligonucleotide is tested for its ability to hybridize to a target polynucleotide (such as mRNA).

In some examples expression and oligonucleotide activity can be determined by mRNA reverse transcription quantitative real-time PCR (RT-qPCR). For example, RNA can be extracted and purified from cells which have been incubated with a candidate oligonucleotide. cDNA is then synthesized from isolated RNA and RT-qPCR can be performed, using methods and reagents known the art. In one example, RNA can be purified from cells using the ISOLATE II RNA Mini Kit (Bioline) and cDNA can be synthesized from isolated RNA using the High-Capacity cDNA Archive kit (Thermo Fisher Scientific) according to the manufacturer's instructions. RT-qPCR can be performed using the Power SYBR Green Master Mix (Thermo Fisher Scientific) on the HT7900 and QuantStudio 6 RT-PCR system (Thermo Fisher Scientific), according to manufacturer's instructions.

Testing for Inhibition of cGAS Activity

Some aspects of the present invention involve testing for inhibition of cGAS activity which can be determined using any method known in the art. In some embodiments, cGAS activity in cells may be measured by expression and/or secretion of one or more pro-inflammatory cytokines or chemokines (e.g. Interferon-β, IP-10), cGAMP levels, activation or expression of transcription factors (e.g. NF-κB) and/or binding or activation of an interferon-stimulated response element (ISRE).

The ability of an oligonucleotide to inhibit cGAS activity can, for example, be analysed by incubating cells which express cGAS with an oligonucleotide, then stimulating said cells with a cGAS agonist (e.g., 70-bp interferon stimulating DNA or ISD70; 45-bp interferon stimulating DNA or ISD45), and analysing the overall cGAS response in the cell population, or analysing the proportion of cells having cGAS-positive activity after a defined period of time.

In such examples, inhibition of cGAS activity can be identified by observation of an overall decreased cGAS response of the cell population, or a lower proportion of cells having cGAS-positive activity as compared to positive control condition in which cells are treated with cGAS agonist in the absence of the oligonucleotide (or in the presence of an appropriate control inhibitory agent). In one example, THP-1 or HT-29 cells are transfected with ISD70 and incubated with an oligonucleotide. cGAS activity can then be determined by cytokine (e.g., IP-10 and/or IFNβ) expression (e.g., gene and/or protein expression) and/or secretion levels, such as by ELISA. A similar assay can be performed with LL171 cells transfected with ISD45. In another example, LL171 cells (mouse L929 cells) expressing an IFN stimulated response element (ISRE)-Luciferase reporter are incubated with an oligonucleotide, and then stimulated with ISD45. cGAS activity can be determined by a luciferase assay, which measures activated IFNβ by luminescence. cGAS activity can also be analysed by measuring cGAMP levels, for example by ELISA. By way of example, cGAS enzymatic activity may be assessed in vitro using recombinant cGAS and then measuring activity thereof by way of a cGAMP ELISA.

### Testing for Inhibition of TLR9 Activity

Some aspects of the present invention involve testing for inhibition of TLR9 activity which can be determined using any method known in the art. In some embodiments TLR9 activity in cells may be measured by expression and/or secretion of one or more pro-inflammatory cytokines (e.g. TNFα, IL-6), and/or activation or expression of transcription factors (e.g. NF-κB).

The ability of an oligonucleotide to inhibit TLR9 activity can, for example, be analysed by incubating cells which express TLR9 with an oligonucleotide, then stimulating said cells with a TLR9 agonist (e.g., CpG ODN2006), and analysing the overall TLR9 response in the cell population, or analysing the proportion of cells having TLR9-positive activity after a defined period of time.

In such examples, inhibition of TLR9 activity can be identified by observation of an overall decreased TLR9 response of the cell population, or a lower proportion of cells having TLR9-positive activity as compared to positive control condition in which cells are treated with TLR9 agonist in the absence of the oligonucleotide (or in the presence of an appropriate control inhibitory agent). In one example, HEK cells are transfected with TLR9 and a NF-κB reporter and incubated with an oligonucleotide and then stimulated with a TLR9 agonist. TLR9 activity can then be determined by a luciferase assay. TLR9 activity can also be analysed by measuring cytokine levels (e.g., IFN, IL-6, TNF and IL-12), for example by ELISA.

### Testing for Inhibition of TLR3 Activity

Some embodiments of the methods of the present invention involve testing for inhibition of TLR3 activity which can be determined using any method known in the art. In some embodiments, TLR3 activity in cells may be measured by expression and/or secretion of one or more pro-inflammatory cytokines (e.g., IFNβ), and/or activation or expression of transcription factors (e.g., NF-κB).

The ability of an oligonucleotide to inhibit TLR3 activity can, for example, be analysed by incubating cells which express TLR3 with an oligonucleotide, then stimulating said cells with a TLR3 agonist, and analysing the overall TLR3 response in the cell population, or analysing the proportion of cells having TLR3-positive activity after a defined period of time.

In such examples, inhibition of TLR3 activity can be identified by observation of an overall decreased TLR3 response of the cell population, or a lower proportion of cells having TLR3-positive activity as compared to positive control condition in which cells are treated with TLR3 agonist in the absence of the oligonucleotide (or in the presence of an appropriate control inhibitory agent). In one example, HEK293 cells stably expressing TLR3 cells are transfected with a pNF-κB-Luc4 reporter, incubated with an oligonucleotide, and then stimulated with a double stranded RNA molecule (e.g., polyl.C). TLR3 activity can be determined by a luciferase assay, which measures activated NF-κB by luminescence. TLR3 activity can also be analysed by measuring cytokine levels, for example by ELISA.

### Testing for Inhibition of TLR7 Activity

Some embodiments of the methods of the present invention involve testing for inhibition of TLR7 activity which can be determined using any method known in the art. In some embodiments, TLR7 activity in cells may be measured by expression and/or secretion of one or more pro-inflammatory cytokines (e.g. TNFα, IP-10), and/or activation or expression of transcription factors (e.g. NF-κB).

The ability of an oligonucleotide to inhibit TLR7 activity can, for example, be analysed by incubating cells which express TLR7 with an oligonucleotide, then stimulating said cells with a TLR7 agonist (e.g., R848, guanosine or an immunostimulatory ssRNA such as B-406-AS), and analysing the overall TLR7 response in the cell population, or analysing the proportion of cells having TLR7-positive activity after a defined period of time.

In such examples, inhibition of TLR7 activity can be identified by observation of an overall decreased TLR7 response of the cell population, or a lower proportion of cells having TLR7-positive activity as compared to positive control condition in which cells are treated with TLR7 agonist in the absence of the oligonucleotide (or in the presence of an appropriate control inhibitory agent). In one example, 293XLhTLR7 (referred to as HEK-TLR7) cells are transfected with pNF-κB-Luc4 reporter, incubated with an oligonucleotide, and then stimulated with R848. TLR7 activity can be determined by a luciferase assay, which measures activated NF-κB by luminescence. TLR7 activity can also be analysed by measuring cytokine levels, for example by ELISA. In another example, primary bone marrow derived macrophages (BMDMs) from wild-type mice are incubated with an oligonucleotide, and then stimulated with guanosine. Alternatively, such cells may express a constitutively active form of TLR7 (e.g., Tlr7^{Y264H}). TLR7 activity may then be assessed by the gene or protein expression of TLR7-responsive genes, such as Tnfα and Oas3.

### Testing for Potentiating TLR8 Activity

Some embodiments of the methods of the present invention involve testing for potentiation of TLR8 activity which can be determined using any method known in the art. In some embodiments TLR8 activity in cells may be measured by expression and/or secretion of one or more pro-inflammatory cytokines (e.g. TNFα, IP-10), and/or activation or expression of transcription factors (e.g. NF-κB).

The ability of an oligonucleotide to potentiate TLR8 activity can, for example, be analysed by incubating cells which express TLR8 with an oligonucleotide, then stimulating said cells with a TLR8 agonist, and analysing the overall TLR8 response in the cell population, or analysing the proportion of cells having TLR8-positive activity after a defined period of time.

In such examples, potentiation of TLR8 activity can be identified by observation of an overall increased TLR8 response of the cell population, or a higher proportion of cells having TLR8-positive activity as compared to a negative control condition in which cells are treated with TLR8 agonist in the absence of the oligonucleotide (or in the presence of an appropriate control non-potentiating agent). In one example, 293XLhTLR8 (referred to as HEK-TLR8) cells are transfected with pNF-κB-Luc4 reporter, incubated with an oligonucleotide, and then stimulated with R848. TLR8 activity can be determined by a luciferase assay, which measures activated NF-κB by luminescence. TLR8 activity can also be analysed by measuring cytokine levels, for example by ELISA.

‘Potentiation’ refers to an increase in a functional property relative to a control condition. Potentiation of TLR8 activity may be greater than about 100%, e.g. about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 20 fold or about 50 fold. Preferably, the level of TLR8 potentiation is between about 2 fold and 50 fold, between about 2 fold and 20 fold, and/or between about 5 fold and 20 fold greater.

### Testing for Inhibition of TLR8 Activity

Some embodiments of the methods of the present invention involve testing for inhibition of TLR8 activity which can be determined using any method known in the art. In some embodiments TLR8 activity in cells may be measured by expression and/or secretion of one or more pro-inflammatory cytokines (e.g. TNFα, IP-10), and/or activation or expression of transcription factors (e.g. NF-κB).

The ability of an oligonucleotide to inhibit TLR8 activity can, for example, be analysed by incubating cells which express TLR8 with an oligonucleotide, then stimulating said cells with a TLR8 agonist, and analysing the overall TLR8 response in the cell population, or analysing the proportion of cells having TLR8-positive activity after a defined period of time.

In such examples, inhibition of TLR8 activity can be identified by observation of an overall decreased TLR8 response of the cell population, or a lower proportion of cells having TLR8-positive activity as compared to a positive control condition in which cells are treated with TLR8 agonist in the absence of the oligonucleotide (or in the presence of an appropriate control inhibitory agent). In one example, 293XLhTLR8 (referred to as HEK-TLR8) cells are transfected with pNF-κB-Luc4 reporter, incubated with an oligonucleotide, and then stimulated with R848. TLR8 activity can be determined by a luciferase assay, which measures activated NF-κB by luminescence. TLR8 activity can also be analysed by measuring cytokine levels, for example by ELISA.

### Uses

Oligonucleotides of the invention are designed to be administered to an animal. For this purpose, the oligonucleotide can be conjugated with another molecule, such as a further nucleic acid (e.g., a mRNA molecule), a peptide, a carrier agent, a therapeutic agent, and the like. In one example, the animal is a vertebrate. For example, the animal can be a mammal, avian, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the mammal is a human.

Oligonucleotides of the invention can be used to target any gene/polynucleotide/function of interest. Alternatively, the oligonucleotides of the invention may be synthetic and do not specifically target any naturally occurring gene or polynucleotide. As such, the oligonucleotides may exhibit little or no inhibitory activity with respect to expression of a target gene or polynucleotide.

Typically, the oligonucleotide is used to modify a trait of an animal, more typically to treat or prevent a disease. In a preferred embodiment, the disease will benefit from the animal not being able to mount a cGAS, a TLR3, a TLR7, TLR8 and/or TLR9 response following administration of the oligonucleotide, in particular where the cGAS response, the TLR7 response and/or the TLR9 response is inhibited. In an alternative embodiment, the disease will benefit from the animal being able to mount an increased TLR8 response following administration of the oligonucleotide.

Diseases which can be treated or prevented using an oligonucleotide of the invention include, but are not limited, to cancer (for example breast cancer, ovarian cancer, cancers of the central nervous system, gastrointestinal cancer, bladder cancer, skin cancer, lung cancer, head and neck cancers, haematological and lymphoid cancers, bone cancer) rare genetic diseases, neuromuscular and neurological diseases (for example, spinal muscular atrophy, Duchenne muscular dystrophy, Huntington's disease, Batten disease, Parkinson's disease, amyotrophic lateral sclerosis, Ataxia-telangiectasia, cerebral palsy) viruses (for example, cytomegalovirus, hepatitis C virus, Ebola haemorrhagic fever virus, human immunodeficiency virus, coronaviruses), cardiovascular disease (for example, familial hypercholesterolemia, hypertriglyceridemia), autoimmune and inflammatory diseases (for example arthritis, lupus, pouchitis, psoriasis, asthma), and non-alcoholic and alcoholic fatty liver diseases. The autoimmune or inflammation diseases may be acute or chonic. In one embodiment, the inflammation may be temporal in nature, for example associated with or caused by an infection.

In particular embodiments, the disease to be treated or prevented using an oligonucleotide of the invention demonstrates increased, excessive or abnormal cGAS expression, activity and/or signalling. Such diseases may include Huntington's disease, Parkinson's diseases, motor-neurone disease (MND), amyotrophic lateral sclerosis (ALS), prion disease, frontotemporal dementia, Traumatic brain injury, Alzheimer's disease, Acute pancreatitis, Silica-induced fibrosis, Age dependent macular degeneration, Aicardi-Goutibres syndrome, myocardial infarction, heart failure, Polyarthritis/foetal and neonatal anaemia, Systemic lupus erythematosus, Acute Kidney Injury, Alcohol-related liver disease, Non-Alcohol-fatty liver disease, silica driven lung inflammation, chronic obstructive pulmonary disease, brain injury after ischemic stroke, sepsis, Non-alcoholic steatohepatitis (NASH), cancer, sickle cell disease, Inflammatory bowel disease, type 2 diabetes mellitus, over-nutrition-induced obesity, COVID-19, hematopoietic disorders, aging-associated inflammation, Cutibacterium acnes Infection, Hepatitis B, posterior-segment eye diseases, arthritis, rheumatoid arthritis, emphysema, colorectal cancer, skin cancer, metastases, and breast cancer, albeit without limitation thereto.

In other embodiments, the disease to be treated or prevented using an oligonucleotide of the invention demonstrates increased, excessive or abnormal TLR9 expression, activity and/or signalling, such as a cancer, an autoimmune disorder, an inflammatory disorder, an autoimmune connective tissue disease (ACTD) and/or a neurodegenerative disorder. Exemplary diseases include psoriasis, arthritis, alopecia universalis, acute disseminated encephalomyelitis, Addison's disease, allergy, ankylosing spondylitis, antiphospholipid antibody syndrome, arteriosclerosis, atherosclerosis, autoimmune haemolytic anaemia, autoimmune hepatitis, Bullous pemphigoid, Chagas' disease, chronic obstructive pulmonary disease, coeliac disease, cutaneous lupus erythematosus (CLE), dermatomyositis, diabetes, dilated cardiomyopathy (DC), endometriosis, Goodpasture's syndrome, Graves' disease, Guillain-Barre syndrome, Hashimoto's disease, hidradenitis suppurativa, idiopathic thrombocytopenic purpura, inflammatory bowel disease, interstitial cystitis, morphea, multiple sclerosis (S), myasthenia gravis, myocarditis, narcolepsy, neuromyotonia, pemphigus, pernicious anaemia, polymyositis, primary biliary cirrhosis, rheumatoid arthritis (RA), schizophrenia, Sjogren's syndrome, systemic lupus erythematosus (SLE), systemic sclerosis, temporal arteritis, vasculitis, vitiligo, vulvodynia, Wegener's granulomatosis, traumatic pain, neuropathic pain and acetaminophen toxicity, breast cancer, cervical squamous cell carcinoma, gastric carcinoma, glioma, hepatocellular carcinoma, lung cancer, melanoma, prostate cancer, recurrent glioblastoma, recurrent non-Hodgkin lymphoma and colorectal cancer, albeit without limitation thereto.

A role for cGAS signalling in cellular senescence has recently been established (see, e.g., Yang et al., 2017) and the presence of senescent cells in an individual may contribute to aging and aging-related dysfunction (see, e.g., Capisi, 2005). Accordingly, one broad aspect of the invention resides in a method for treating, reducing the likelihood of, or delaying the onset of a senescence-associated disease, disorder or condition, such as cancer, cardiovascular diseases, neurodegenerative diseases and aging and aging-related diseases, disorders or conditions, in a subject in need thereof, including the step of administering to the subject a therapeutically effective amount of an oligonucleotide described herein. Suitably, the oligonucleotide inhibits cGAS activity when administered to the subject.

In a related form, the invention further provides a method of preventing or inhibiting senescence in a cell, including the step of contacting the cell with an effective amount of an oligonucleotide described herein. Suitably, the oligonucleotide inhibits cGAS activity in the cell when contacted therewith. It will also be appreciated by the skilled person that the current method may be performed in vitro or in vivo.

The cell may be any known in the art that is capable of senescence. By way of example, the cell can be an immune cell, such as T cells (e.g., CD4+, CD8+, NK and regulatory T cells), B cells, natural killer cells, neutrophils, eosinophils, mast cells, basophils, monocytes, macrophages and dendritic cells. In other examples, the cell can be a stem cell, such as a haematopoietic stem cell. Suitably, the cell, such as the immune cell or stem cell, is for use in a cell based therapy.

By way of example, the immune cells may be used for adoptive cell transfer, such as tumour-infiltrating lymphocytes (TIL) or gene-modified T cells expressing novel T cell receptors (TCR) or chimeric antigen receptors (CAR). Adoptive cell therapy (ACT) can refer to the transfer of cells, most commonly immune-derived cells, back into the sae patient or into a new recipient host with the goal of transferring the immunologic functionality and characteristics into the new host. To this end, the immune cell, such as a T cell, preferably a CD8+ T cell, may be engineered or modified to express a T cell receptor having specificity to a desired antigen, such as a tumour cell antigen. For example, the immune cell may comprise a chimeric antigen receptor (CAR) having specificity to a desired antigen, such as a tumour-specific chimeric antigen receptor (CAR).

In another form, the oligonucleotides of the invention may be used in methods of preventing or inhibiting inflammation associated with administration of a therapeutic oligonucleotide, such as those known in the art, to a subject. In particular, the oligonucleotides described herein may be used in the prevention or inhibition of inflammation mediated by one or more nucleic acid sensors (e.g., TLR3, TLR7, TLR8, TLR9, cGAS, RIG-I, MDA5, PKR) during or following administration of the therapeutic oligonucleotide. It is envisaged that the inflammation may involve or include any cells, tissues or organs of the body. In particular embodiments, the inflammation is or comprises hepatic inflammation. To this end, the therapeutic oligonucleotide may be conjugated to N-acetylgalactosamine (GalNAc), which enhances asialoglycoprotein receptor (ASGR)-mediated uptake into liver hepatocytes (Nair et al., 2014), and thereby enabling their specific targeting to the liver.

In certain examples, the oligonucleotides of the invention, and more particularly those described herein that exhibit TLR7-inhibitory activity, may be utilised to prevent or inhibit a TLR7-dependent inflammatory response associated with the administration of an RNA molecule in vitro or in vivo. More particularly, the RNA molecule may be part of RNA-based therapeutic agent, such as an mRNA vaccine. In this regard, the oligonucleotide can at least partly inhibit the engagement or sensing of these therapeutic RNA molecules by TLR7. The oligonucleotides of the invention may therefore minimise the need for the use of modified bases, such as pseudo-uridines, and/or other modifications that reduce the immunogenicity of mRNA molecules for their inclusion in mRNA vaccine compositions.

In certain examples, the oligonucleotides of the invention, and more particularly those described herein that exhibit TLR8-inhibitory activity, may be utilised to prevent or inhibit a TLR8-dependent inflammatory response associated with the administration of an RNA molecule in vitro or in vivo. More particularly, the RNA molecule may be part of RNA-based therapeutic agent, such as an mRNA vaccine. In this regard, the oligonucleotide can at least partly inhibit the engagement or sensing of these therapeutic RNA molecules by TLR8. The oligonucleotides of the invention may therefore minimise the need for the use of modified bases, such as pseudo-uridines, and/or other modifications that reduce the immunogenicity of mRNA molecules for their inclusion in mRNA vaccine compositions.

As such, the oligonucleotides of the invention may be a component or included within an immunogenic composition, such as an RNA or mRNA vaccine composition, as are known in the art. The term “RNA vaccine” refers to vaccines comprising RNA that encodes one or more nucleotide sequences encoding antigens capable of inducing an immune response in a mammal. mRNA vaccines are described, for example, in International Patent Application Nos. PCT/US2015/027400 and PCT/US2016/044918, herein incorporated by reference in their entirety.

In a particular form, the present invention provides an immunogenic composition, such as a vaccine composition, comprising an RNA molecule and an oligonucleotide provided herein. Suitably, the oligonucleotide of the immunogenic composition exhibits TLR7, TLR8 and/or TLR3 inhibitory activity as described herein. In certain embodiments, the oligonucleotide of the immunogenic composition exhibits TLR7 inhibitory activity. In certain embodiments, the oligonucleotide of the immunogenic composition exhibits TLR8 inhibitory activity. In certain embodiments, the oligonucleotide of the immunogenic composition exhibits TLR3 inhibitory activity. In some embodiments, the oligonucleotide of the immunogenic composition exhibits TLR7 and TLR3 inhibitory activity. In some embodiments, the oligonucleotide of the immunogenic composition exhibits TLR7 and TLR8 inhibitory activity. The immunogenic composition is suitably for use in a method of: (a) inducing an immune response in a subject; and/or (b) preventing, treating or ameliorating an infection, disease or condition in a subject in need thereof.

It will be appreciated that mRNA vaccines provide unique therapeutic alternatives to peptide- or DNA-based vaccines. When the mRNA vaccine is delivered to a cell, the mRNA will be processed into a polypeptide or peptide by the intracellular machinery which can then process the polypeptide or peptide into immunogenic fragments capable of stimulating an immune response. To this end, the oligonucleotide may be included as a separate or discrete component and/or conjugated with an RNA or mRNA molecule of the vaccine composition. With respect to such embodiments, the RNA molecule of the RNA vaccine may be unmodified or substantially unmodified (e.g., does not include any modified bases). Alternatively, the RNA molecule may contain one or more modifications that typically enhance stability, such as modified nucleotides, modified sugar phosphate backbones, and 5′ and/or 3′ untranslated regions (UTR).

Additionally, the RNA molecule may be included or incorporated within a delivery, transfer or carrier system of the immunogenic composition, as are known in the art. For example, the mRNA or RNA molecule of the immunogenic composition may be encapsulated or complexed in nanoparticles, and more particularly lipid nanoparticles. According to various embodiments, suitable nanoparticles include, but are not limited to polymer based carriers, such as polyethylenimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocry stalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic poly conjugates) and dry powder formulations.

In some embodiments, the oligonucleotide is included in the immunogenic composition separate from the carrier system. In other embodiments, the oligonucleotide is included or incorporated within the carrier system of the immunogenic composition, such as incorporated into a lipid nanoparticle together with the RNA molecule of the RNA vaccine.

In particular examples, therapeutically effective amounts of the therapeutic oligonucleotide and the oligonucleotide of the invention may be administered simultaneously, concurrently, sequentially, successively, alternately or separately in any particular combination and/or order.

Examples of target genes (polynucleotides) of oligonucleotides of the invention include, but not limited to, PLK1ERBB2, PIK3CA, ERBB3, HDAC1, MET, EGFR, TYMS, TUBB4B, FGFR2, ESRI, FASN, CDK4, CDK6, NDUFB4, PPAT, NDUFB7, DNMT1, BCL2, ATP1A1, HDAC3, FGFR1, NDUFS2, HDAC2, NDUFS3, HMGCR, IGF1R, AKT1, BCL2L1, CDK2, MTOR, PDPK1, CSNK2A1, PIK3CB, CDK12, MCL1, ATR, PLK4, MEN1, PTK2, FZD5, KRAS, WRN, CREBBP, NRAS, MAT2A, RHOA, TPX2, PPP2CA, ALDOA, RAE1, SKP1, ATP5A1, EIF4G1, CTNNB1, TFRC, CDH1, CCNE1, CLTC, METAP2, GRB2, MDM4, SLC16A1, FERMT2, ENO1, STX4, SF3B1, RBBP4, FEN1, MRPL28, CCNA2, PTPN11, SAE1, KMT2D, APC, CAD, NAMPT, OGT, HSPA8, USP5, CSNK1A1, PGD, VRK1, SEPSECS, SUPT4H1, DNAJC9, TRIAPI, DLD, PTPN7, VDAC1, STAT3, TCEB2, ADSL, GMPS, DHPS, METAPI, TAF13, CFL1, SCD, RBM39, PGAM1, FNTB, PPP2R1A, ARF1, UBE2T, UMPS, MYC, PRMT5, EIF4G2, SKP2, STAG2, ATF4, WDR77, ILK, METTL16, SOD1, DDX6, FURIN, AARS, FNTA, PABPC1, RANBP2, CDC25B, SLC2A1, CENPE, ADAR, CDC42, RNF31, CCNC, PRIM1, SLC38A2, SNUPN, PDCD6IP, RTN4IP1, VMP1, TGFBR1, TXN, UBE2N, UAP1, RAC1, GGPS1, RAB10, RAB6A, TPI1, RPE, THG1L, UBE2D3, RHEB, PKM, GMNN, HGS, NCKAP1, NUP98, SMARCA2, RNF4, DDX39B, ACLY, XPO1, PPP1R8, YAP1, MTHFD1, LPAR1, TAF1, UROD, STXBP3, HSP90B1, VHL, EFR3A, FECH, MRPL44, AIFM1, MAGOH, MRPL17, SUZ12, RNMT, RAB1B, PNPT1, RAD1, WDR48, PITRM1, MRPL47, AP2M1, EIF4A1, UBE2C, LONP1, VPS4A, SNRNP25, TUBGCP6, DNM2, UBE2M, EXOSC9, TAF1B, CDC37, ATP6V1G1, POP1, JUP, PRPS1, GPX4, CFLAR, CHMP4B, ACTB, ACTRIA, PTPN23, SHC1, TRPM7, SLC4A7, HSPD1, XRN1, WDR1, ITGB5, UBR4, ATP5B, CPD, TUFM, MYH9, ATP5F1, ATP6V1C1, SOD2, PFAS, NFE2L2, ARF4, ITGAV, DHX36, KIF18A, DDX5, XRCC5, DNAJC11, ZBTB8OS, NCL, SDHB, ATP5C1, NDC1, SNF8, CUL3, SLC7A1, ASNA1, EDF1, TMED10, CHMP6, ARIH1, DDOST, RPL28, DIMT1, CMPK1, PPIL1, PPA2, SMAD7, CEP55, MVD, MVK, PDS5A, KNTC1, CAPZB, GMPPB, TPT1, ACIN1, SAR1A, TAF6L, PTBP1, PAK2, CRKL, NHLRC2, IN080, SLC25A3, ACTR3, DDX3X, HUWEl, TBCA, IK, SSBP1, ARPC4, SLC7A5, OSGEP, PDCD2, TRAF2, SNAP23, RPN1, EIF5A, GEMIN4, BMPR1A, AHCYL1, CHMP5, TRAPPC1, LRP8, ARID2, UBE2L3, STAMBP, KDSR, UQCRC2, PNN, USP7, TBCD, ATP6VOE1, PCYT1A, TAZ, POLRMT, CELSR2, TERF1, BUB1, YRDC, SMG6, TBX3, SLC39A10, IPO13, CDIPT, UBA5, EMC7, FERMT1, VEZT, CCND1, CCND2, FPGS, JUN, PPM1D, PGGT1B, NPM1, GTF2A1, MBTPS1, HMGCS1, LRR1, HSD17B12, LCE2A, NUP153, FOSL1, IRS2, CYB5R4, PMPCB, ARHGEF7, TRRAP, NRBP1, ARMC7, MOCS3, TIPARP, SEC61A1, PFDN5, MYB, IRF4, STX5, MYCN, FOXA1, SOX10, GATA3, ZEB2, MYBL2, MFN2, TBCB, KLF4, TRIM37, CEBPA, STAG1, POU2AF1, HYPK, FLIl, NCAPD2, MAF, NUP93, RBBP8, HJURP, SMARCB1, SOCS3, GRWD1, NKX2-1, FDXR, SPDEF, SBDS, SH3GL1, KLF5, CNOT3, ZNF407, CPSF1, RPTOR, EXT1, SMC1A, GUKI, TIMM23, FAU, ACO2, ALG1, CCNL1, SCAP, SRSF6, SPAG5, SOX9, LDB1, ASPM, LIG1, TFDP1, RPAIN, CENPA, MIS12, ILF3, HSCB, ERCC2, SOX2, ARFRP1, PMF1, POLR3E, MAD2L2, PELP1, NXT1, WDHD1, ZWINT, E2F3, FZR1, JUNB, OGDH, NOB1, SKA3, TACC3, UTP14A, XRN2, SMG5, IDH3A, CIAO1, COQ4, ZFP36L1, CDCA5, PRKRA, PFDN6, PAKIIPI, PSTK, EDC4, UTP18, TOMM22, CASC5, PTTG1, RBBP5, PPP1R12A, FARS2, FOXM1, SIN3A, BUB1B, GNB1L, SMC5, SARS2, SYNCRIP, IPPK, FANCD2, WDR46, FANCI, DCP2, RFC2, RNF20, DMAP1, MED23, MBNL1, CTPS1, TBP, MMS19, RAD51C, CDS2, NONO, USP18, PARS2, FBXW11, SUMO2, RRP12, FAM50A, URB2, MCM4, SLC25A28, IP07, MAX, SFSWAP, SBNO1, DPAGT1, TINF2, BRCA2, NUP50, RPIA, EP400, IKBKAP, KIF14, RTTN, CCDC115, GEMIN6, WWTR1, BCS1L, GTF3A, SCYL1, NELFB, DDX39A, TRA2B, SYVN1, ISL1, CYB5B, ACSL3, DPH3, E2F1, IREB2, SREBF1, SMC6, IRF8, ID1, PDCD11, SNAPC2, TIMM17A, ANAPC10, NUP85, SEH1L, VBP1, NUDC, MTX2, RPP25L, ISYl, LEMD2, ATP5D, EXOSC2, TAF1C, PPIL4, SEPHS2, HNRNPH1, CTR9, CDC26, TIMM13, FAM96B, CEBPZ, UFL1, ZNF236, COPG1, TPR, MIOS, UBE2G2, MED12, GTF3C1, PPP2R2A, UBIADI, WTAP, MYBBPlA, NUP88, NELFCD, WDR73, RTCB, CEP192, GTF3C5, LENG1, RINT1, MED24, COX6B1, DCTN6, SLC25A38, LYRM4, STRAP, TTF2, DDX27, GTF2F1, ZNHIT2, BCLAF1, WDR18, GTF2H2C, NDE1, TIMM9, CHMP7, IPO11, TGIF1, NOC4L, EXOSC6, WDR24, INTS6, DDX41, UBE2S, ARGLUI, SHOC2, ATP5J, CSTF2, RPP30, NHP2, GRHL2, RPL22L1, WDR74, UTP23, CCDC174, RPP21, UBE2J2, GEMIN8, ATP6V0B, KIAA1429, PNO1, MED22, ENY2, THOC7, DDX19A, SUGP1, PELO, ELAC2, CHCHD4, RNPC3, INTS3, PSMG4, UQCRC1, TAFlA, TSR1, UTP6, TRMT5, EIF1AD, GTF3C2, DCTN3, GPS1, WDR7, EXOSC8, KANSL1, SPRTN, KANSL3, EXOSC5, PRCC, TRNAU1AP, EIF3J, TAMM41, HAUS6, OIP5, HAUS5, TAF6, MRPS22, MRPS34, WBP11, COG8, DHX38, DNLZ, LAGE3, FUBP1, MED26, SLC7A6OS, MARS2, RBM28, ASCC3, PSMG3, TUBGCP5, PCF11, orLASIL.

In an embodiment, the gene to be targeted includes PKN3, VEGFA, KIF11, MYC, EPHA2, KRAS (G12), ERBB3, BIRC5, HIF1A, BCL2, STAT3, AR, EPAS1, BRCA2, or CLU.

Examples of commercial oligonucleotides which can be modified as described herein include, but are not limited to, inclisiran, mipomersen (Kynamro), nusinersen (Spinraza), eteplirsen (Exondys51), miravirsen (SPC3649), RG6042 (IONIS-HTTRx), inotersen, volanesorsen, golodirsen (Vyondys53), fomivirsen (Vitravene), patisiran, givosiran, danvatirsen and IONIS-AR-2.5Rx.

### Compositions

Oligonucleotides of the disclosure may be admixed, encapsulated, conjugated (such as fused) or otherwise associated with other molecules, molecule structures or mixtures of compounds, resulting in, for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921, 5,354,844, 5,416,016, 5,459,127, 5,521,291, 5,543,158, 5,547,932, 5,583,020, 5,591,721, 4,426,330, 4,534,899, 5,013,556, 5,108,921, 5,213,804, 5,227,170, 5,264,221, 5,356,633, 5,395,619, 5,416,016, 5,417,978, 5,462,854, 5,469,854, 5,512,295, 5,527,528, 5,534,259, 5,543,152, 5,556,948, 5,580,575, and 5,595,756.

Oligonucleotides of the disclosure may be administered in a pharmaceutically acceptable carrier. The pharmaceutically acceptable carrier may be solid or liquid. Useful examples of pharmaceutically acceptable carriers include, but are not limited to, diluents, solvents, surfactants, excipients, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure.

In one embodiment, the pharmaceutical carrier is water for injection (WFI) and the pharmaceutical composition is adjusted to pH 7.4, 7.2-7.6. In one embodiment, the salt is a sodium or potassium salt.

The oligonucleotides may contain chiral (asymmetric) centres or the molecule as a whole may be chiral. The individual stereoisomers (enantiomers and diastereoisomers) and mixtures of these are within the scope of the present disclosure.

Oligonucleotides of the disclosure may be pharmaceutically acceptable salts, esters, or salts of the esters, or any other compounds which, upon administration are capable of providing (directly or indirectly) the biologically active metabolite. The term “pharmaceutically acceptable salts” as used herein refers to physiologically and pharmaceutically acceptable salts of the oligonucleotide that retain the desired biological activities of the parent compounds and do not impart undesired toxicological effects upon administration. Examples of pharmaceutically acceptable salts and their uses are further described in U.S. Pat. No. 6,287,860.

Oligonucleotides of the disclosure may be prodrugs or pharmaceutically acceptable salts of the prodrugs, or other bioequivalents. The term “prodrugs” as used herein refers to therapeutic agents that are prepared in an inactive form that is converted to an active form (i.e., drug) upon administration by the action of endogenous enzymes or other chemicals and/or conditions. In particular, prodrug forms of the oligonucleotide of the disclosure are prepared as SATE [(S acetyl-2-thioethyl) phosphate] derivatives according to the methods disclosed in WO 93/24510, WO 94/26764 and U.S. Pat. No. 5,770,713.

A prodrug may, for example, be converted within the body, e. g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl—, Br—, I—, F—, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3.beta.-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides (as well as mRNA vaccines) to cells (see, e.g., U.S. Pat. Nos. 5,855,910; 5,851,548; 5,830,430; 5,780,053; 5,767,099; Lewis et al., 1996; Hope et al., 1998). Other lipid compositions which can be used to facilitate uptake of the instant oligonucleotides can be used in connection with the methods of the invention. In addition to those listed above, other lipid compositions are also known in the art and include, e.g., those taught in U.S. Pat. Nos. 4,235,871; 4,501,728; 4,837,028; 4,737,323.

In one embodiment, lipid compositions can further comprise agents, e.g., viral proteins to enhance lipid-mediated transfections of oligonucleotides. In another embodiment, N-substituted glycine oligonucleotides (peptoids) can be used to optimize uptake of oligonucleotides.

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

In a further embodiment, the oligonucleotide is attached to a targeting moiety such as N-acetylgalactosamine (GalNAc), an antibody, antibody-like molecule or aptamer (see, for example, Toloue and Ford (2011) and Esposito et al. (2018)).

### Administration

In one embodiment, the oligonucleotide of the disclosure is administered systemically. As used herein “systemic administration” is a route of administration that is either enteral or parenteral.

As used herein “enteral” refers to a form of administration that involves any part of the gastrointestinal tract and includes oral administration of, for example, the oligonucleotide in tablet, capsule or drop form; gastric feeding tube, duodenal feeding tube, or gastrostomy; and rectal administration of, for example, the oligonucleotide in suppository or enema form.

As used herein “parenteral” includes administration by injection or infusion. Examples include, intravenous (into a vein), intra-arterial (into an artery), intramuscular (into a muscle), intra-cardiac (into the heart), subcutaneous (under the skin), intraosseous infusion (into the bone marrow), intradermal, (into the skin itself), intrathecal (into the spinal canal), intraperitoneal (infusion or injection into the peritoneum), intra-vesical (infusion into the urinary bladder). transdermal (diffusion through the intact skin), transmucosal (diffusion through a mucous membrane), inhalational.

In one embodiment, administration of the pharmaceutical composition is subcutaneous.

The oligonucleotide may be administered as single dose or as repeated doses on a period basis, for example, daily, once every two days, three, four, five, six seven, eight, nine, ten, eleven, twelve, thirteen or fourteen days, once weekly, twice weekly, three times weekly, every two weeks, every three weeks, every month, every two months, every three months to six months or every 12 months.

In one embodiment, administration is 1 to 3 times per week, or once every week, two weeks, three weeks, four weeks, or once every two months.

In one embodiment, administration is once weekly.

In one embodiment, a low dose administered for 3 to 6 months, such as about 25-50 mg/week for at least three to six months and then up to 12 months and chronically.

Illustrative doses are between about 10 to 5,000 mg. Illustrative doses include 25, 50,100,150, 200, 1,000, 2,000 mg. Illustrative doses include 1.5 mg/kg (about 50 to 100 mg) and 3 mg/kg (100-200 mg), 4.5 mg/kg (150-300 mg), 10 mg/kg, 20 mg/kg or 30 mg/kg. In one embodiment doses are administered once per week. Thus in one embodiment, a low dose of approximately 10 to 30, or 20 to 40, or 20 to 28 mg may be administered to subjects typically weighing between about 25 and 65 kg. In one embodiment the oligonucleotide is administered at a dose of less than 50 mg, or less than 30 mg, or about 25 mg per dose to produce a therapeutic effect.

### TABLES

TABLE 1
Various oligonucleotides used in this study (all in 5′-3′). UPPERCASE alone
for DNA, ‘m’ indicates 2′OMe base, and * denotes the phosphorothioate backbone.

| Name | Sequence |
|---|---|
| [cGAS]ASO | mA*mU*mG*mG*mC*C*T*T*T*C*C*G*T*G*C*mC*mA*mA*mG*mG |
| 1-146 | |
| [cGAS]ASO | mU*mC*mC*mG*mG*C*C*T*C*G*G*A*A*G*C*mU*mC*mU*mC*mU |
| 2-168 | |
| [cGAS]ASO | mG*mC*mA*mU*U*C*C*G*T*G*C*G*G*A*A*mG*mC*mC*mU*mU |
| 3-200 | |
| [cGAS]ASO | mG*mG*mC*mC*mG*A*A*C*T*T*T*C*C*C*G*mC*mC*mU*mU*mA |
| 4-277 | |
| [cGAS]ASO | mG*mG*mU*mC*mU*T*G*G*C*T*T*C*G*T*G*mG*mA*mG*mC*mA |
| 5-521 | |
| [cGAS]ASO | mG*mG*mA*mG*mC*T*T*C*G*A*G*G*C*C*C*mC*mA*mG*mG*mC |
| 6-616 | |
| [cGAS]ASO | mG*mG*mU*mG*mG*T*C*C*A*C*A*A*C*C*C*mC*mU*mU*mU*mC |
| 7-697 | |
| [cGAS]ASO | mC*mA*mU*mU*mA*G*G*T*G*C*A*G*A*A*A*mU*mC*mU*mU*mC |
| 8-793 | |
| [cGAS]ASO | mU*mU*mC*mU*mG*G*G*G*A*C*T*T*C*C*A*mG*mU*mU*mU*mA |
| 9-829 | |
| [cGAS]ASO | mU*mG*mA*mU*mU*C*C*A*A*A*G*C*C*A*G*mG*mG*mU*mU*mA |
| 10-1098 | |
| [cGAS]ASO | mC*mU*mU*mU*mA*G*T*C*G*T*A*G*T*T*G*mC*mU*mU*mC*mC |
| 11-1185 | |
| ASO11Mut1 | mC*mU*mU*mU*mA*G*T*C*G*T*A*G*T*T*G*mU*mC*mU*mC*mU |
| ASO11Mut2 | mU*mC*mC*mG*mG*G*T*C*G*T*A*G*T*T*G*mC*mU*mU*mC*mC |
| ASO2up | mU*mC*mC*mG*mG*C*C*T*C*G*G*A*G*T*C*mU*mC*mC*mA*mU |
| ASO2down | mU*mC*mC*mG*mG*C*C*T*C*G*G*C*A*G*A*mU*mA*mU*mC*mG |
| ASO2-Mut1 | mU*mC*mC*mG*mG*C*C*T*C*G*G*G*A*G*A*mU*mC*mU*mC*mU |
| C2 | mG*mC*mA*mG*mU*C*T*C*C*A*T*G*T*C*C*mC*mA*mG*mG*mC |
| C2-Mut1 | mG*mC*mG*mG*mU*A*T*C*C*A*T*G*T*C*C*mC*mA*mG*mG*mC |
| C2-Mut2 | mG*mC*mG*mG*mU*A*T*A*C*A*G*G*T*C*C*mC*mA*mG*mG*mC |
| C2-Mut1v1 | mG*mC*mU*mG*mU*T*T*C*C*A*T*G*T*C*C*mC*mA*mG*mG*mC |
| C2-Mut1v2 | mG*mC*mU*mG*mU*G*T*C*C*A*T*G*T*C*C*mC*mA*mG*mG*mC |
| C2-Mut1v3 | mG*mC*mC*mG*mU*T*T*C*C*A*T*G*T*C*C*mC*mA*mG*mG*mC |
| C2-Mut1v4 | mG*mC*mC*mG*mU*G*T*C*C*A*T*G*T*C*C*mC*mA*mG*mG*mC |
| C2-ASO2-A | mG*mC*mG*mG*mU*A*T*C*C*A*T*A*G*T*C*mU*mC*mC*mA*mU |
| C2-ASO2-B | mG*mC*mG*mG*mU*A*T*C*C*A*T*C*A*G*A*mU*mA*mU*mC*mG |
| C2-Mut1-PS | G*C*G*G*T*A*T*C*C*A*T*G*T*C*C*C*A*G*G*C (SEQ ID NO: 353) |
| [C2]Mut1- | mG*mG*mU*A*T*C*C*C*C*C*C*C*C*C*C*C*C*C*C*C |
| dC | |
| [C2]Mut1- | mC*mG*mU*T*T*C*C*C*C*C*C*C*C*C*C*C*C*C*C*C |
| v3-dC | |
| dC20 | C*C*C*C*C*C*C*C*C*C*C*C*C*C*C*C*C*C*C*C (SEQ ID NO: 354) |
| ASO2-Cy3 | mU*mC*mC*mG*mG*C*C*T*C*G*G*A*A*G*C*mU*mC*mU*mC*mU/3Cy3Sp/ |
| A151 | T*T*A* G*G*G* T*T*A* G*G*G* T*T*A* G*G*G* T*T*A* G*G*G (SEQ ID |
| | NO: 355) |
| C151 | T*T*C* A*A*A* T*T*C* A*A*A* T*T*C* A*A*A* T*T*C* A*A*A (SEQ ID |
| | NO: 356) |
| IRS661 | T*G*C* T*T*G* C*A*A* G*C*T* T*G*C* A*A*G* C*A (SEQ ID NO: 357) |
| IRS957 | T*G*C* T*T*G* A*C*A* T*C*C* T*G*G* A*G*G* G*G*T* T*G*T (SEQ ID |
| | NO: 358) |
| [HPRT]551 | mC*mU*mU*mC*mC*A*C*A*A*T*C*A*A*G*A*mC*mA*mU*mU*mC |
| [HPRT]660 | mC*mU*mU*mC*mG*T*G*G*G*G*T*C*C*T*T*mU*mU*mC*mA*mC |
| [HPRT]661 | mA*mC*mU*mU*mC*G*T*G*G*G*G*T*C*C*T*mU*mU*mU*mC*mA |
| [HPRT]662 | mC*mA*mC*mU*mU*C*G*T*G*G*G*G*T*C*C*mU*mU*mU*mU*mC |
| [HPRT]664 | mA*mA*mC*mA*mC*T*T*C*G*T*G*G*G*G*T*mC*mC*mU*mU*mU |
| [HPRT]665 | mC*mA*mA*mC*mA*C*T*T*C*G*T*G*G*G*G*mU*mC*mC*mU*mU |
| [HPRT]666 | mC*mC*mA*mA*mC*A*C*T*T*C*G*T*G*G*G*mG*mU*mC*mC*mU |
| ODN 2006 | T*C*G* T*C*G* T*T*T* T*G*T* C*G*T* T*T*T* G*T*C* G*T*T (SEQ ID |
| | NO: 359) |
| ODN 1826 | T*C*C* A*T*G* A*C*G* T*T*C* C*T*G* A*C*G* T*T (SEQ ID NO: 360) |
| ISD45-FWD | TAC AGA TCT ACT AGT GAT CTA TGA CTG ATC TGT ACA TGA TCT ACA |
| | (SEQ ID NO: 361) |
| ISD45-REV | TGT AGA TCA TGT ACA GAT CAG TCA TAG ATC ACT AGT AGA TCT GTA |
| | (SEQ ID NO: 362) |
| ISD70-FWD | CCA TCA GAA AGA GGT TTA ATA TTT TTG TGA GAC CAT CGA AGA GAG |
| | AAA GAG ATA AAA CTT TTT TAC GAC T |
| | (SEQ ID NO: 363) |
| ISD70-REV | AGT CGT AAA AAA GTT TTA TCT CTT TCT CTC TTC GAT GGT CTC ACA |
| | AAA ATA TTA AAC CTC TTT CTG ATG G |
| | (SEQ ID NO: 364) |

TABLE 1A
Unmodified oligonucleotides described in Table 1.

| Name | Sequence |
|---|---|
| Unmodified | AUGGCCTTTCCGTGCCAAGG (SEQ ID NO: 9) |
| [cGAS]ASO1-146 | |
| Unmodified | UCCGGCCTCGGAAGCUCUCU (SEQ ID NO: 10) |
| [cGAS]ASO2-168 | |
| Unmodified | GCAUUCCGTGCGGAAGCCUU (SEQ ID NO: 11) |
| [cGAS]ASO3-200 | |
| Unmodified | GGCCGAACTTTCCCGCCUUA (SEQ ID NO: 12) |
| [cGAS]ASO4-277 | |
| Unmodified | GGUCUTGGCTTCGTGGAGCA (SEQ ID NO: 13) |
| [cGAS]ASO5-521 | |
| Unmodified | GGAGCTTCGAGGCCCCAGGC (SEQ ID NO: 14) |
| [cGAS]ASO6-616 | |
| Unmodified | GGUGGTCCACAACCCCUUUC (SEQ ID NO: 15) |
| [cGAS]ASO7-697 | |
| Unmodified | CAUUAGGTGCAGAAAUCUUC (SEQ ID NO: 16) |
| [cGAS]ASO8-793 | |
| Unmodified | UUCUGGGGACTTCCAGUUUA (SEQ ID NO: 17) |
| [cGAS]ASO9-829 | |
| Unmodified | UGAUUCCAAAGCCAGGGUUA (SEQ ID NO: 18) |
| [cGAS]ASO10- | |
| 1098 | |
| Unmodified | CUUUAGTCGTAGTTGCUUCC (SEQ ID NO: 19) |
| [cGAS]ASO11- | |
| 1185 | |
| Unmodified | CUUUAGTCGTAGTTGUCUCU (SEQ ID NO: 50) |
| ASO11Mut1 | |
| Unmodified | UCCGGGTCGTAGTTGCUUCC (SEQ ID NO: 51) |
| ASO11Mut2 | |
| Unmodified | UCCGGCCTCGGAGTCUCCAU (SEQ ID NO: 52) |
| ASO2up | |
| Unmodified | UCCGGCCTCGGCAGAUAUCG (SEQ ID NO: 163) |
| ASO2down | |
| Unmodified ASO2- | UCCGGCCTCGGGAGAUCUCU (SEQ ID NO: 53) |
| Mut1 | |
| Unmodified C2 | GCAGUCTCCATGTCCCAGGC (SEQ ID NO: 30) |
| Unmodified C2- | GCGGUATCCATGTCCCAGGC (SEQ ID NO: 42) |
| Mut 1 | |
| Unmodified C2- | GCGGUATACAGGTCCCAGGC (SEQ ID NO: 43) |
| Mut2 | |
| Unmodified C2- | GCUGUTTCCATGTCCCAGGC (SEQ ID NO: 44) |
| Mut1v1 | |
| Unmodified C2- | GCUGUGTCCATGTCCCAGGC (SEQ ID NO: 45) |
| Mut1v2 | |
| Unmodified C2- | GCCGUTTCCATGTCCCAGGC (SEQ ID NO: 46) |
| Mut1v3 | |
| Unmodified C2- | GCCGUGTCCATGTCCCAGGC (SEQ ID NO: 47) |
| Mut1v4 | |
| Unmodified C2- | GCGGUATCCATAGTCUCCAU (SEQ ID NO: 48) |
| ASO2-A | |
| Unmodified C2- | GCGGUATCCATCAGAUAUCG (SEQ ID NO: 49) |
| ASO2-B | |
| Unmodified C2- | GCGGTATCCATGTCCCAGGC (SEQ ID NO: 353) |
| Mut1-PS | |
| Unmodified | GGUATCCCCCCCCCCCCCCC (SEQ ID NO: 54) |
| [C2]Mut1-dC | |
| Unmodified | CGUTTCCCCCCCCCCCCCCC (SEQ ID NO: 460) |
| [C2]Mut1-v3-dC | |
| Unmodified dC20 | CCCCCCCCCCCCCCCCCCC (SEQ ID NO: 354) |
| Unmodified ASO2- | UCCGGCCTCGGAAGCUCUCU (SEQ ID NO: 157) |
| Cy3 | |
| Unmodified A151 | TTAGGGTTAGGGTTAGGGTTAGGG (SEQ ID NO: 355) |
| Unmodified C151 | TTCAAATTCAAATTCAAATTCAAA (SEQ ID NO: 356) |
| Unmodified | TGCTTGCAAGCTTGCAAGCA (SEQ ID NO: 357) |
| IRS661 | |
| Unmodified | TGCTTGACATCCTGGAGGGGTTGT (SEQ ID NO: 358) |
| IRS957 | |
| Unmodified | CUUCCACAATCAAGACAUUC (SEQ ID NO: 158) |
| [HPRT]551 | |
| Unmodified | CUUCGTGGGGTCCTTUUCAC (SEQ ID NO: 461) |
| [HPRT] 660 | |
| Unmodified | ACUUCGTGGGGTCCTUUUCA (SEQ ID NO: 159) |
| [HPRT] 661 | |
| Unmodified | CACUUCGTGGGGTCCUUUUC (SEQ ID NO: 462) |
| [HPRT] 662 | |
| Unmodified | AACACTTCGTGGGGTCCUUU (SEQ ID NO: 463) |
| [HPRT]664 | |
| Unmodified | CAACACTTCGTGGGGUCCUU (SEQ ID NO: 160) |
| [HPRT]665 | |
| Unmodified | CCAACACTTCGTGGGGUCCU (SEQ ID NO: 20) |
| [HPRT] 666 | |
| Unmodified ODN | TCGTCGTTTTGTCGTTTTGTCGTT (SEQ ID NO: 359) |
| 2006 | |
| Unmodified ODN | TCCATGACGTTCCTGACGTT (SEQ ID NO: 360) |
| 1826 | |
| Unmodified | TACAGATCTACTAGTGATCTATGACTGATCTGTACATGATCTACA |
| ISD45-FWD | (SEQ ID NO: 361) |
| Unmodified | TGTAGATCATGTACAGATCAGTCATAGATCACTAGTAGATCTGTA |
| ISD45-REV | (SEQ ID NO: 362) |
| Unmodified | CCATCAGAAAGAGGTTTAATATTTTTGTGAGACCATCGAAGAGAGA |
| ISD70-FWD | AAGAGATAAAACTTTTTTACGACT (SEQ ID NO: 363) |
| Unmodified | AGTCGTAAAAAAGTTTTATCTCTTTCTCTCTTCGATGGTCTCACAAAA |
| ISD70-REV | ATATTAAACCTCTTTCTGATGG (SEQ ID NO: 364) |

TABLE 2
80 ASO screen data.

| [Transcript | | | | | | |
|---|---|---|---|---|---|---|
| Name] | | | | | | |
| and Seq | | | cGAS- | TLR9- | TLR7- | TLR8- |
| Name | Sequence | Position | 100 nM | 500 nM | 100 nM | 500 nM |
| †[CDKN2B- | mU*mU*mA*mA*mA*T*A*A* | B5 | 52.20 | 98.18 | 25.66 | 6.43 |
| AS1]132 | T*C*T*A*G*T*T*mU*mG*mA* | | | | | |
| | mA*mG | | | | | |
| †[CDKN2B- | mG*mU*mG*mU*mC*C*T*T*C | F3 | 58.18 | 98.99 | 18.05 | 1.60 |
| AS1]1415 | *A*T*G*C*T*T*mU*mG*mG* | | | | | |
| | mA*mU | | | | | |
| [CDKN2B- | mA*mG*mA*mA*mA*G*A*A* | F5 | 49.09 | 68.12 | 42.99 | 2.78 |
| AS1]1519 | G*C*A*A*A*G*A*mU*mU*mC | | | | | |
| | *mA*mA | | | | | |
| #[CDKN2B- | mC*mC*mU*mA*mG*A*A*A* | C5 | 60.90 | 42.44 | 57.20 | 4.94 |
| AS1]1522 | G*A*A*G*C*A*A*mA*mG*mA | | | | | |
| | *mU*mU | | | | | |
| [CDKN2B- | mG*mU*mC*mA*mA*A*C*C*T | E4 | 68.94 | 68.13 | 23.64 | 1.85 |
| AS1]1528 | *A*G*A*A*A*G*mA*mA*mG* | | | | | |
| | mC*mA | | | | | |
| #[CDKN2B- | mG*mA*mU*mU*mA*A*A*A* | A6 | 89.36 | 44.97 | 21.92 | 1.48 |
| AS1]1773 | C*A*G*A*T*T*A*mA*mU*mA | | | | | |
| | *mC*mA | | | | | |
| #[CDKN2B- | mG*mG*mA*mU*mU*A*A*A* | C6 | 50.11 | 27.79 | 27.16 | 1.67 |
| AS1]1774 | A*C*A*G*A*T*T*mA*mA*mU | | | | | |
| | *mA*mC | | | | | |
| [CDKN2B- | mA*mG*mG*mA*mU*T*A*A* | G4 | 88.54 | 63.48 | 36.28 | 2.63 |
| AS1] 1775 | A*A*C*A*G*A*T*mU*mA*mA | | | | | |
| | *mU*mA | | | | | |
| [CDKN2B- | mA*mG*mA*mU*mU*A*T*C*T | F6 | 55.80 | 78.72 | 37.10 | 6.26 |
| AS1]2130 | *T*C*T*T*T*T*mA*mA*mU*m | | | | | |
| | U*mU | | | | | |
| [CDKN2B- | mA*mA*mG*mA*mU*T*A*T*C | G5 | 73.50 | 59.77 | 42.09 | 5.62 |
| AS1]2131 | *T*T*C*T*T*T*mU*mA*mA*m | | | | | |
| | U*mU | | | | | |
| [CDKN2B- | mA*mA*mA*mG*mA*T*T*A*T | E5 | 74.59 | 63.14 | 49.28 | 5.04 |
| AS1]2132 | *C*T*T*C*T*T*mU*mU*mA*m | | | | | |
| | A*mU | | | | | |
| [CDKN2B- | mA*mA*mA*mA*mG*A*T*T* | B6 | 57.68 | 80.95 | 47.41 | 4.68 |
| AS1]2133 | A*T*C*T*T*C*T*mU*mU*mU* | | | | | |
| | mA*mA | | | | | |
| [CDKN2B- | mG*mA*mA*mA*mA*G*A*T* | H5 | 51.49 | 61.11 | 34.44 | 1.87 |
| AS1]2134 | T*A*T*C*T*T*C*mU*mU*mU* | | | | | |
| | mU*mA | | | | | |
| [CDKN2B- | mU*mG*mU*mG*mA*A*A*A* | G6 | 40.72 | 62.16 | 34.51 | 4.97 |
| AS1]2137 | G*A*T*T*A*T*C*mU*mU*mC | | | | | |
| | *mU*mU | | | | | |
| [CDKN2B- | mU*mU*mG*mU*mG*A*A*A* | H6 | 69.36 | 61.15 | 35.02 | 1.94 |
| AS1]2138 | A*G*A*T*T*A*T*mC*mU*mU | | | | | |
| | *mC*mU | | | | | |
| #[CDKN2B- | mC*mU*mU*mG*mU*G*A*A* | E6 | 33.75 | 34.91 | 85.33 | 6.02 |
| AS1]2139 | A*A*G*A*T*T*A*mU*mC*m | | | | | |
| | U*mU*mC | | | | | |
| †[CDKN2B- | mG*mG*mU*mG*mG*C*C*A* | D4 | 48.58 | 97.04 | 39.07 | 0.98 |
| AS1]2196 | C*A*G*G*C*A*A*mC*mG*mU | | | | | |
| | *mC*mA | | | | | |
| [CDKN2B- | mA*mA*mG*mG*mU*G*G*C* | H3 | 60.48 | 84.28 | 57.85 | 0.78 |
| AS1]2198 | C*A*C*A*G*G*C*mA*mA*mC | | | | | |
| | *mG*mU | | | | | |
| [CDKN2B- | mA*mG*mG*mC*mC*T*C*C*A | B3 | 62.85 | 81.24 | 44.73 | 4.66 |
| AS1]2218 | *G*T*G*T*C*T*mU*mC*mU* | | | | | |
| | mC*mC | | | | | |
| [CDKN2B- | mC*mA*mG*mG*mC*C*T*C*C* | H2 | 99.38 | 66.99 | 54.11 | 3.89 |
| AS1]2219 | A*G*T*G*T*C*mU*mU*mC*mU | | | | | |
| | *mC | | | | | |
| [CDKN2B- | mG*mU*mC*mC*mC*A*G*G* | G3 | 74.22 | 67.61 | 19.01 | 2.17 |
| AS1]2223 | C*C*T*C*C*A*G*mU*mG*mU | | | | | |
| | *mC*mU | | | | | |
| #[CDKN2B- | mC*mC*mA*mU*mG*T*C*C* | C4 | 36.34 | 48.79 | 41.39 | 1.46 |
| AS1]2227 | C*A*G*G*C*C*T*mC*mC*m | | | | | |
| | A*mG*mU | | | | | |
| †[CDKN2B- | mU*mC*mU*mC*mC*A*T*G*T* | C3 | 94.78 | 118.93 | 43.45 | 9.17 |
| AS1]2230 | C*C*C*A*G*G*mC*mC*mU*m | | | | | |
| | C*mC | | | | | |
| [CDKN2B; | mG*mU*mC*mU*mC*C*A*T*G | D2 | 84.16 | 77.80 | 23.75 | 1.25 |
| AS1]2231 | *T*C*C*C*A*G*mG*mC*mC* | | | | | |
| | mU*mC | | | | | |
| †[CDKN2B- | mA*mG*mU*mC*mU*C*C*A*T | B2 | 88.13 | 122.20 | 35.77 | 2.28 |
| AS1]2232 | *G*T*C*C*C*A*mG*mG*mC* | | | | | |
| | mC*mU | | | | | |
| #[CDKN2B- | mC*mA*mG*mU*mC*T*C*C*A | A2 | 75.92 | 55.13 | 47.75 | 3.81 |
| AS1]2233 | *T*G*T*C*C*C*mA*mG*mG* | | | | | |
| | mC*mC | | | | | |
| [CDKN2B- | mG*mC*mA*mG*mU*C*T*C* | C2 | 10.02 | 90.04 | 20.30 | 2.25 |
| AS1]2234 | C*A*T*G*T*C*C*mC*mA*m | | | | | |
| | G*mG*mC | | | | | |
| [CDKN2B- | mA*mG*mC*mA*mG*T*C*T* | F2 | 28.75 | 73.18 | 25.50 | 4.93 |
| AS1]2235 | C*C*A*T*G*T*C*mC*mC*m | | | | | |
| | A*mG*mG | | | | | |
| [CDKN2B- | mA*mA*mG*mC*mA*G*T*C*T* | G2 | 107.34 | 69.71 | 35.69 | 2.62 |
| AS1]2236 | C*C*A*T*G*T*mC*mC*mC*mA | | | | | |
| | *mG | | | | | |
| [CDKN2B- | mA*mA*mA*mG*mC*A*G*T* | E2 | 70.95 | 87.17 | 27.16 | 3.38 |
| AS1]2237 | C*T*C*C*A*T*G*mU*mC*mC* | | | | | |
| | mC*mA | | | | | |
| [CDKN2B- | mA*mG*mU*mG*mG*C*A*C* | B4 | 59.69 | 86.07 | 41.95 | 4.48 |
| AS1]495 | A*T*A*C*C*A*C*mA*mC*mC | | | | | |
| | *mC*mU | | | | | |
| †[CDKN2B- | mG*mU*mG*mU*mU*T*T*T*A | H4 | 66.24 | 98.50 | 23.63 | 1.88 |
| AS1]611 | *A*T*T*T*T*G*mU*mA*mG* | | | | | |
| | mA*mG | | | | | |
| [CDKN2B- | mC*mA*mG*mU*mG*T*T*T*T | F4 | 60.94 | 92.59 | 62.68 | 6.73 |
| AS1]613 | *T*A*A*T*T*T*mU*mG*mU* | | | | | |
| | mA*mG | | | | | |
| [CDKN2B- | mA*mU*mU*mU*mC*C*A*C* | A4 | 52.58 | 63.65 | 32.05 | 4.09 |
| AS1]626 | A*T*G*C*C*C*A*mG*mU*mG | | | | | |
| | *mU*mU | | | | | |
| [CDKN2B- | mU*mA*mU*mU*mU*C*C*A* | E3 | 80.99 | 71.20 | 30.54 | 3.87 |
| AS1]627 | C*A*T*G*C*C*C*mA*mG*mU | | | | | |
| | *mG*mU | | | | | |
| #[CDKN2B- | mA*mA*mU*mU*mU*A*A*A* | D6 | 67.63 | 53.26 | 46.75 | 5.02 |
| AS1]645 | G*C*A*T*G*A*A*mU*mA*mU | | | | | |
| | *mU*mA | | | | | |
| [CDKN2B- | mA*mA*mA*mA*mU*A*A*G*G* | D5 | 94.10 | 67.44 | 68.45 | 0.71 |
| AS1]79 | G*G*A*A*T*A*mG*mG*mG*mG | | | | | |
| | *mA | | | | | |
| [CDKN2B- | mU*mA*mA*mA*mA*T*A*A*G* | A5 | 110.58 | 64.58 | 40.95 | 1.30 |
| AS1]80 | G*G*G*A*A*T*mA*mG*mG*mG | | | | | |
| | *mG | | | | | |
| [CDKN2B- | mA*mU*mA*mU*mC*T*G*C*T* | A3 | 108.01 | 73.46 | 47.52 | 5.89 |
| AS1]831 | G*C*C*C*A*C*mC*mU*mU*m | | | | | |
| | C*mU | | | | | |
| [CDKN2B- | mA*mA*mU*mA*mU*C*T*G* | D3 | 83.17 | 70.72 | 43.66 | 4.45 |
| AS1]832 | C*T*G*C*C*C*A*mC*mC*mU* | | | | | |
| | mU*mC | | | | | |
| [LINC- | mU*mC*mC*mC*mA*T*C*C*C | D9 | 86.62 | 59.80 | 43.08 | 10.88 |
| PINT]101 | *T*T*C*T*G*C*mU*mG*mA*m | | | | | |
| | C*mA | | | | | |
| †[LINC- | mG*mU*mC*mC*mC*A*T*C*C* | H9 | 113.87 | 101.22 | 22.47 | 5.07 |
| PINT]102 | C*T*T*C*T*G*mC*mU*mG*mC | | | | | |
| | *mC | | | | | |
| [LINC- | mG*mG*mU*mC*mC*C*A*T* | F9 | 45.23 | 88.75 | 20.75 | 1.87 |
| PINT]103 | C*C*C*T*T*C*T*mG*mC*mU | | | | | |
| | *mG*mC | | | | | |
| #[LINC- | mU*mC*mU*mG*mG*T*C*C*C | B10 | 48.75 | 56.60 | 29.42 | 5.25 |
| PINT]106 | *A*T*C*C*C*T*mU*mC*mU* | | | | | |
| | mG*mC | | | | | |
| †§[LINC- | mU*mC*mU*mC*mU*G*G*T*C | A9 | 104.65 | 100.51 | 78.25 | 11.42 |
| PINT]108 | *C*C*A*T*C*C*mC*mU*mU*m | | | | | |
| | C*mU | | | | | |
| §[LINC- | mC*mU*mC*mU*mC*T*G*G*T | B9 | 80.22 | 89.85 | 84.97 | 10.10 |
| PINT]109 | *C*C*C*A*T*C*mC*mC*mU* | | | | | |
| | mU*mC | | | | | |
| [LINC- | mU*mC*mU*mC*mU*C*T*G*G | G9 | 92.39 | 74.25 | 43.82 | 11.27 |
| PINT]110 | *T*C*C*C*A*T*mC*mC*mC*m | | | | | |
| | U*mU | | | | | |
| †[LINC- | mU*mU*mC*mU*mC*T*C*T*G | E9 | 77.41 | 98.01 | 26.22 | 9.51 |
| PINT]111 | *G*T*C*C*C*A*mU*mC*mC* | | | | | |
| | mC*mU | | | | | |
| [LINC- | mC*mU*mU*mC*mU*C*T*C*T | B8 | 82.64 | 64.35 | 56.20 | 9.21 |
| PINT]112 | *G*G*T*C*C*C*mA*mU*mC* | | | | | |
| | mC*mC | | | | | |
| #[LINC- | mC*mC*mU*mU*mC*T*C*T*C* | A8 | 103.79 | 55.47 | 60.90 | 3.51 |
| PINT]113 | T*G*GT*C*C*mC*mA*mU*mC | | | | | |
| | *mC | | | | | |
| [LINC- | mC*mC*mC*mU*mU*C*T*C*T | C8 | 84.38 | 62.09 | 49.59 | 8.12 |
| PINT]114 | *C*T*G*G*T*C*mC*mC*mA* | | | | | |
| | mU*mC | | | | | |
| #[LINC- | mA*mC*mC*mC*mU*T*C*T*C | G8 | 89.81 | 32.00 | 43.37 | 7.57 |
| PINT]115 | *T*C*T*G*G*T*mC*mC*mC*m | | | | | |
| | A*mU | | | | | |
| §[LINC- | mC*mA*mC*mC*mC*T*T*C*T* | D10 | 97.19 | 102.85 | 73.30 | 5.97 |
| PINT]116 | C*T*C*T*G*G*mU*mC*mC*mC | | | | | |
| | *mA | | | | | |
| [LINC- | mU*mU*mA*mG*mC*T*C*C*T | C9 | 63.80 | 78.17 | 31.67 | 8.41 |
| PINT] 1222 | *T*G*C*C*T*C*mG*mU*mU* | | | | | |
| | mC*mC | | | | | |
| [LINC- | mG*mU*mC*mU*mC*C*T*C*C | H8 | 114.36 | 77.09 | 25.01 | 7.17 |
| PINT]126 | *A*C*A*C*C*C*mU*mU*mC*m | | | | | |
| | U*mC | | | | | |
| [LINC- | mG*mG*mU*mC*mU*C*C*T*C | D8 | 36.95 | 77.91 | 23.66 | 3.72 |
| PINT]127 | *C*A*C*A*C*C*mC*mU*mU* | | | | | |
| | mC*mU | | | | | |
| †[LINC- | mG*mG*mG*mU*mC*T*C*C* | E8 | 37.83 | 94.51 | 21.47 | 1.23 |
| PINT]128 | T*C*C*A*C*A*C*mC*mC*m | | | | | |
| | U*mU*mC | | | | | |
| [LINC- | mU*mC*mC*mC*mA*A*C*T*C* | G7 | 99.64 | 86.73 | 40.79 | 13.35 |
| PINT]1284 | T*T*C*T*A*A*mC*mU*mC*mG | | | | | |
| KL | *mU | | | | | |
| #[LINC- | mG*mC*mA*mA*mG*G*C*A* | H7 | 30.64 | 53.46 | 19,99 | 2.69 |
| PINT]1315 | G*A*G*A*A*A*C*mU*mC*m | | | | | |
| KL | C*mA*mG | | | | | |
| #[LINC- | mA*mA*mA*mU*mG*T*C*C*T | A11 | 66.46 | 46.72 | 46.45 | 1.56 |
| PINT]148.1 | *G*G*C*C*C*T*mC*mA*mC* | | | | | |
| | mU*mG | | | | | |
| [LINC- | mG*mA*mU*mG*mG*T*T*C* | E10 | 26.05 | 80.47 | 22.32 | 1.01 |
| PINT]1497.1 | C*A*G*T*C*C*C*mU*mC*m | | | | | |
| | U*mU*mC | | | | | |
| #[LINC- | mU*mU*mG*mG*mC*C*T*G*T | C11 | 68.35 | 40.71 | 21.43 | 0.94 |
| PINT]2673.1 | *G*G*A*T*G*C*mU*mU*mU* | | | | | |
| | mG*mU | | | | | |
| #[LINC | mU*mU*mU*mG*mA*A*A*T* | H11 | 84.15 | 54.33 | 35.07 | 4.93 |
| PINT\|2690 | T*C*A*G*A*A*G*mA*mU*mU | | | | | |
| | *mU*mG | | | | | |
| [LINC- | mC*mU*mU*mU*mA*T*A*T*T* | F11 | 92.54 | 84.43 | 54.83 | 6.37 |
| PINT]2755 | A*C*A*A*A*G*mC*mU*mA*mC | | | | | |
| | *mU | | | | | |
| [LINC- | mU*mG*mA*mU*mG*A*T*G* | G10 | 85.85 | 65.76 | 25.50 | 1.57 |
| PINT\|283.1 | C*T*T*G*C*A*G*mG*mA*mG | | | | | |
| | *mG*mC | | | | | |
| †[LINC- | mC*mA*mC*mU*mG*T*A*T*T | E11 | 69.47 | 117.42 | 58.13 | 4.86 |
| PINT]2990 | *T*T*A*T*T*A*mC*mA*mG*m | | | | | |
| | A*mA | | | | | |
| #[LINC- | mU*mG*mA*mC*mA*A*A*A* | G11 | 88.79 | 53.61 | 36.68 | 2.57 |
| PINT]384 | C*A*A*T*A*A*T*mA*mA*mC | | | | | |
| | *mA*mG | | | | | |
| [LINC- | mG*mU*mU*mC*mA*G*T*C*A* | A7 | 93.74 | 77.50 | 17.46 | 1.70 |
| PINT]412 | G*A*T*C*G*C*mU*mG*mG*m | | | | | |
| KL | G*mA | | | | | |
| [LINC- | mU*mG*mU*mU*mU*C*C*C* | B7 | 57.81 | 71.47 | 30.90 | 2.04 |
| PINT]501 | C*G*G*A*G*A*G*mC*mA*mA | | | | | |
| KL | *mU*mG | | | | | |
| †[LINC- | mU*mG*mA*mC*mA*T*T*T*C | C7 | 39.34 | 118.29 | 33.56 | 4.34 |
| PINT]523 | *G*T*G*G*C*T*mC*mC*mU* | | | | | |
| KL | mA*mC | | | | | |
| †[LINC- | mA*mU*mG*mA*mC*A*T*T*T* | H10 | 99.38 | 107.12 | 32.53 | 1.20 |
| PINT]524.1 | C*G*T*G*G*C*mU*mC*mC*m | | | | | |
| | U*mA | | | | | |
| [LINC- | mA*mG*mC*mC*mG*A*A*C* | D7 | 39.92 | 57.46 | 27.70 | 3.73 |
| PINT]587 | A*G*A*A*G*G*A*mG*mC*m | | | | | |
| KL | G*mU*mC | | | | | |
| [LINC- | mG*mU*mC*mC*mG*T*A*C*C | B11 | 86.09 | 58.15 | 22.79 | 3.70 |
| PINT]727.1 | *T*C*C*A*C*C*mC″mA*mC* | | | | | |
| | mC*mG | | | | | |
| †[LINC- | mC*mA*mA*mG*mC*C*C*C*A | F10 | 77.10 | 93.79 | 45.20 | 4.44 |
| PINT]83.1 | *G*C*G*T*T*C*mC*mU*mC* | | | | | |
| | mC*mG | | | | | |
| [LINC- | mC*mC*mC*mU*mA*A*T*G*C | E7 | 89.09 | 86.47 | 58.39 | 8.39 |
| PINT1877 | *T*T*T*C*C*T*mC*mU*mC*m | | | | | |
| KL | C*mA | | | | | |
| #[LINC- | mG*mC*mG*mU*mA*G*T*T*T | F7 | 50.62 | 52.04 | 27.23 | 4.12 |
| PINT]935 | *C*T*C*T*T*C*mC*mU*mC*m | | | | | |
| KL | C*mC | | | | | |
| [LINC- | mU*mG*mG*mC*mG*T*A*G*T | D11 | 70.97 | 79.89 | 32.81 | 4.25 |
| PINT]937.1 | *T*T*C*T*C*T*mU*mC*mC*m | | | | | |
| | U*mC | | | | | |
| †§[LINC- | mC*mC*mU*mU*mC*T*G*C*T* | A10 | 104.33 | 96.69 | 85.57 | 3.71 |
| PINT]94 | G*C*C*A*A*G*mC*mC*mC*mC | | | | | |
| | *mA | | | | | |
| [LINC- | mC*mA*mU*mC*mC*C*T*T*C | F8 | 70.60 | 65.09 | 54.34 | 8.23 |
| PINT]98 | *T*G*C*T*G*C*mC*mA*mA* | | | | | |
| | mG*mC | | | | | |
| ILING- | mC*mC*mA*mU*mC*C*C*T*T | C10 | 85.43 | 92.72 | 52.57 | 5.67 |
| PINT199 | *C*T*G*C*T*G*mC*mC*mA* | | | | | |
| | mA*mG | | | | | |

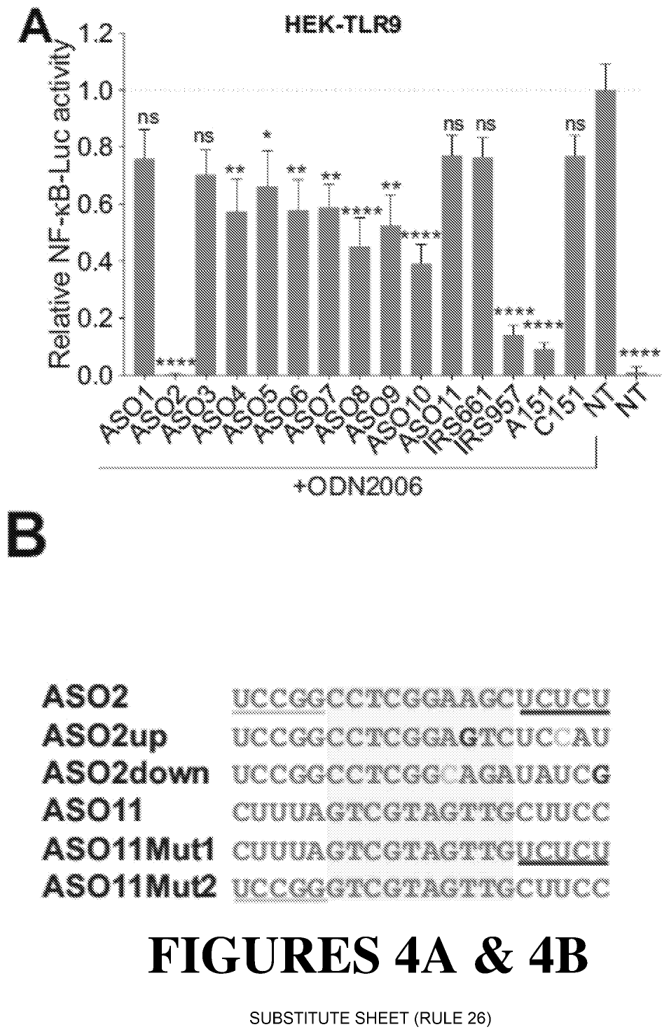

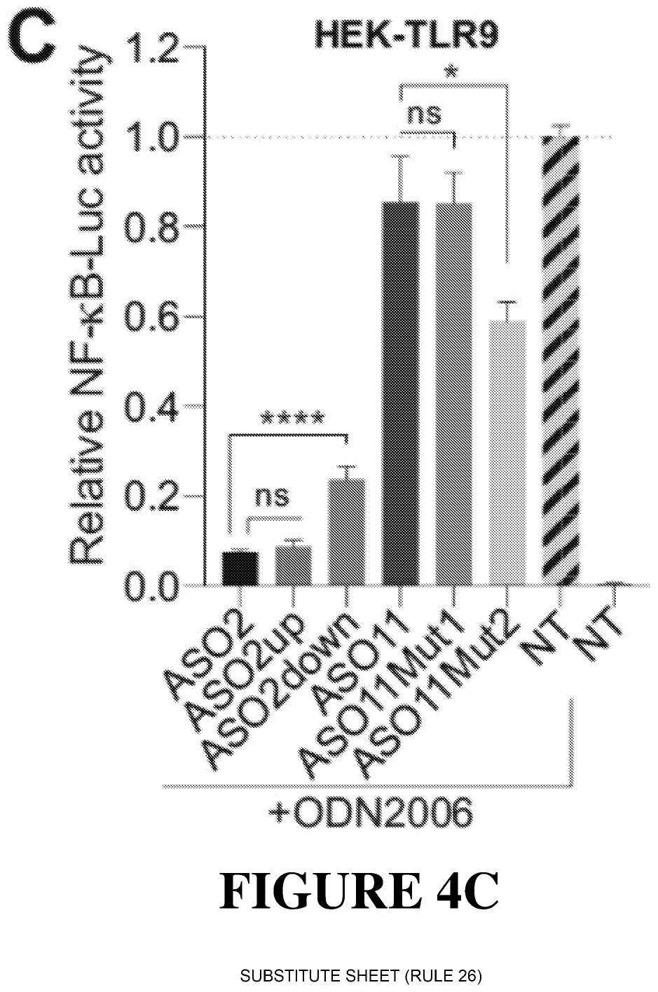

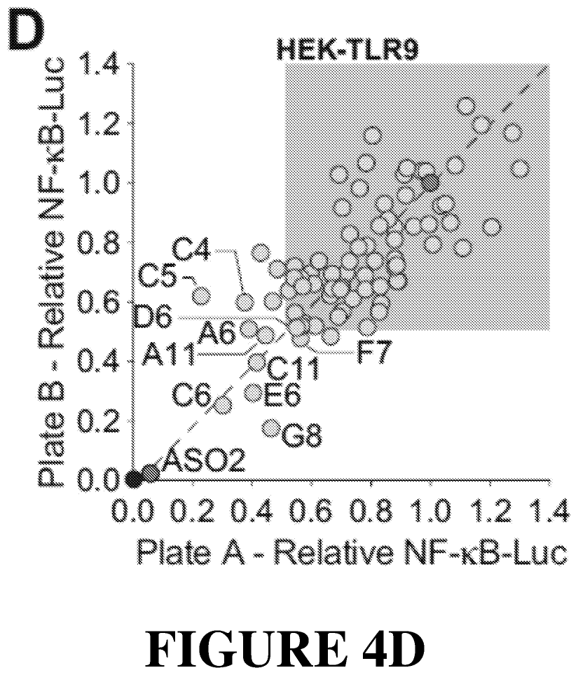

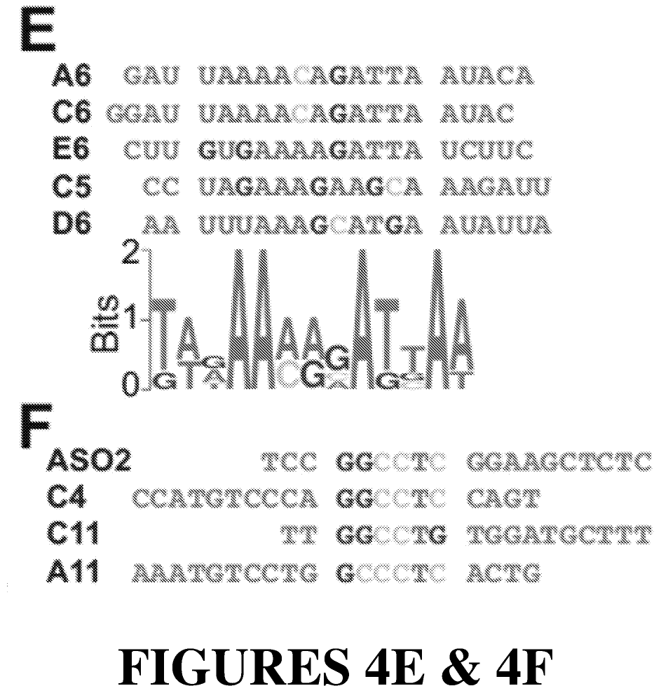

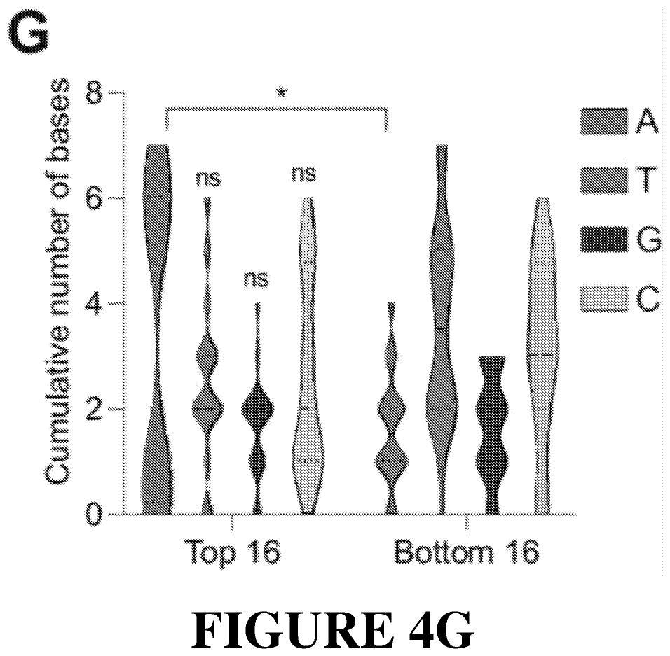

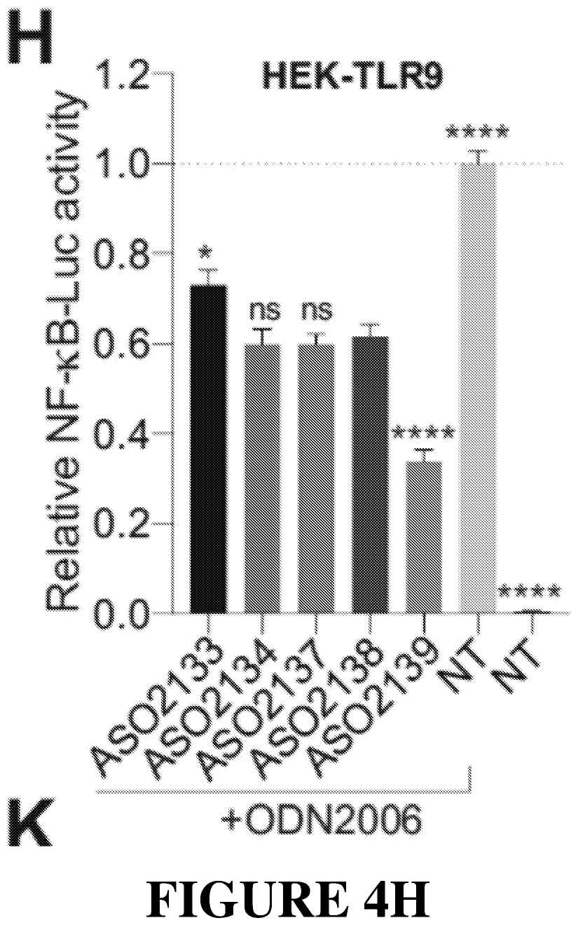

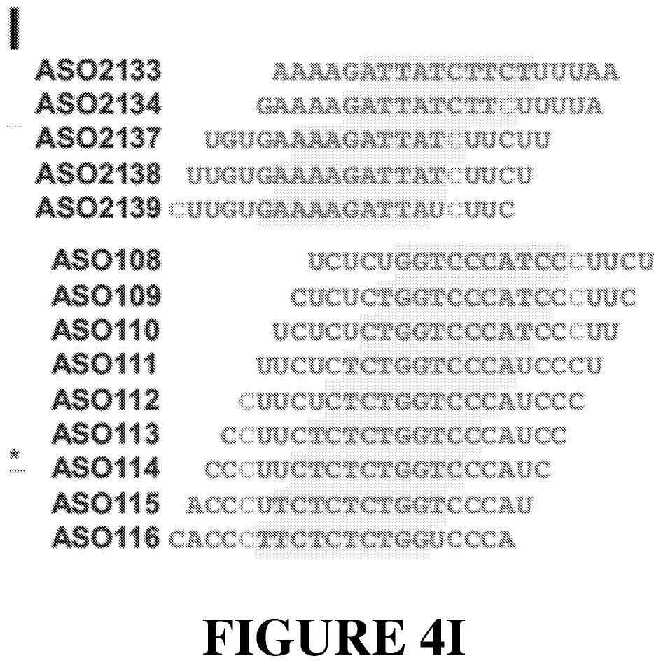

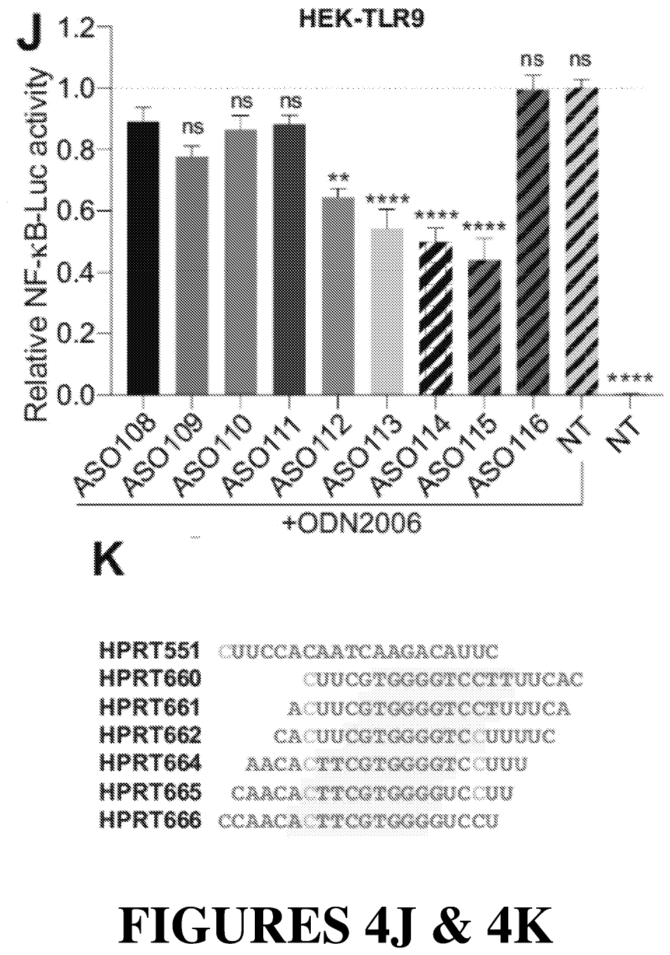

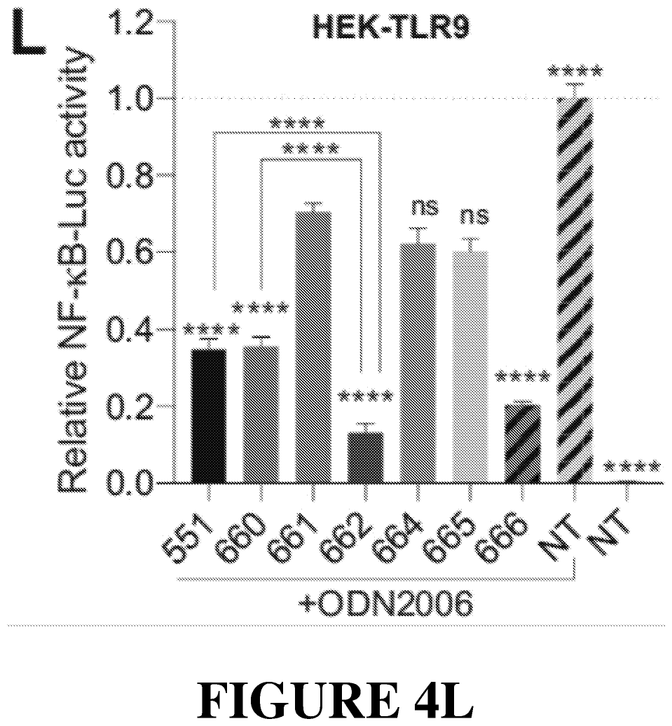

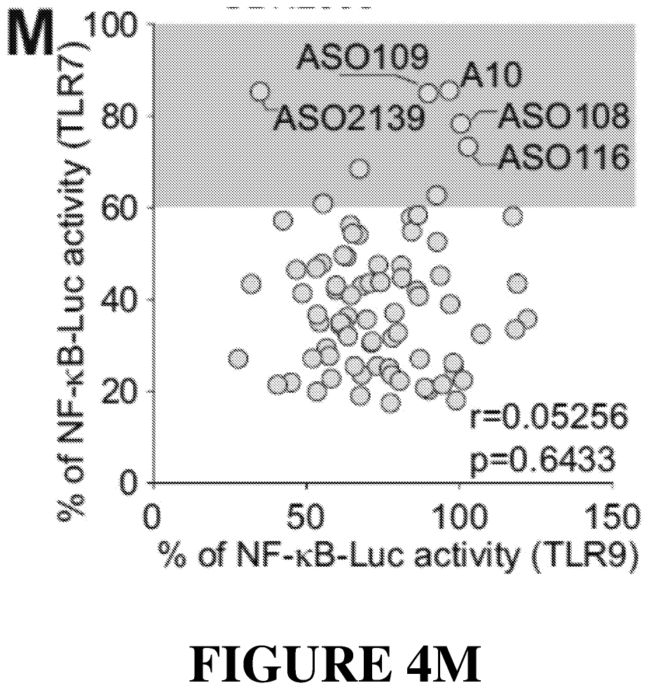

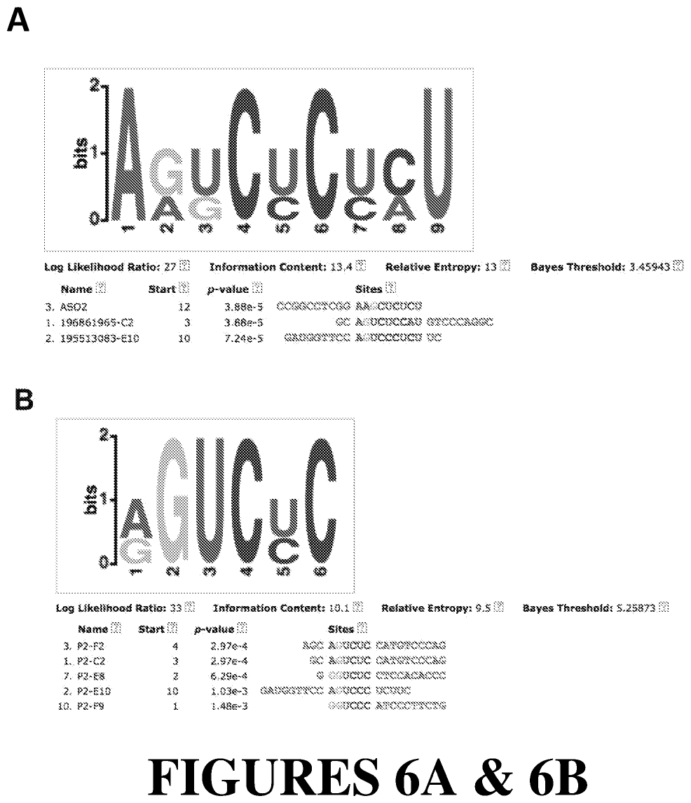

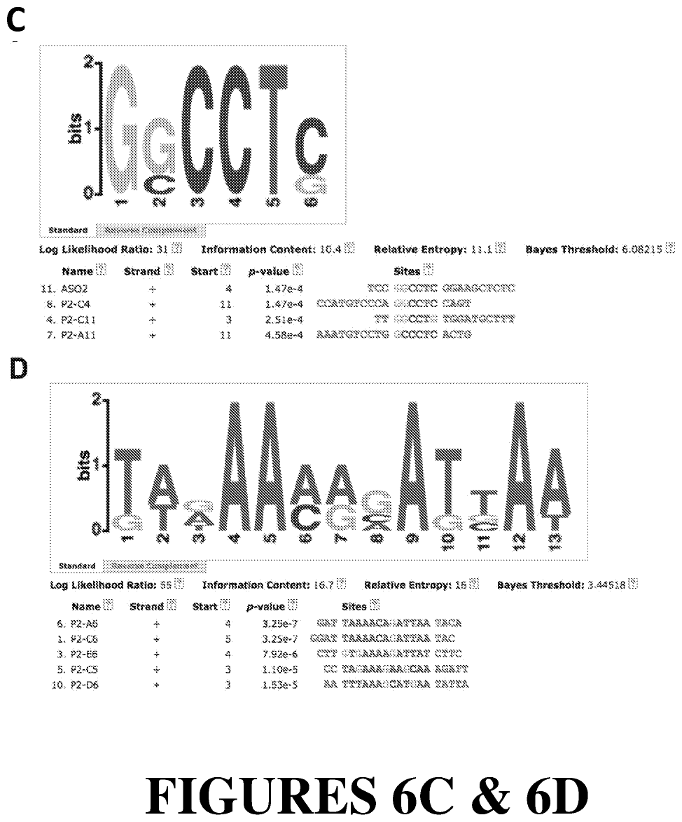

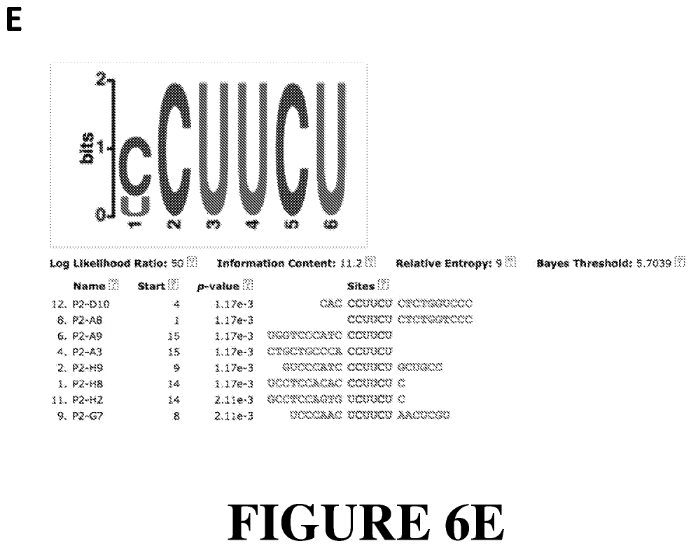

The targeted gene names are provided in brackets (e.g. [CDKN2B-AS1]), followed by the reference position in the target RNA. ASOs were synthesised with the following modifications: UPPERCASE alone for DNA, ‘in’ indicates 2′OMe base modifications, and * denotes the phosphorothioate backbone. The “Position” column denotes the position of the ASOs in the 96 well plate used in the screen. The concentration of the ASOs used in each screen is indicated. Averaged NF-κB-Luciferase or IP-10 levels from each screen are given relative to ISD70 (cGAS), ODN2006 (TLR9), R848 (TLR7/8—Alharbi et al., 2020) conditions, as percentages (TLR7/9/cGAS) or fold increases (TLR8). Bold denotes the 10 strongest cGAS inhibitors used for motif discovery in FIG. 6B. Italic denotes the 17 ASOs with less than 10% inhibition of “ISD70 only” condition used for motif discovery in FIG. 6E. Underlined denotes the 10 most potent TLR9 inhibitors used for motif discovery in FIGS. 6C and 6D. The symbol “#” denotes the 16 most potent TLR9 inhibitors and the symbol “t” the 16 weakest inhibitors of TLR9 used in FIG. 4G. The symbol “§ ” denotes the 4 ASOs inhibiting TLR7/9 and cGAS by less than 30%.

TABLE 2A
Unmodified ASOs described in Table 2.

| [Transcript Name] | |
|---|---|
| and Seq Name | Sequence |
| Unmodified †[CDKN2B- | UUAAATAATCTAGTTUGAAG (SEQ ID NO: 20) |
| AS1]132 | |
| Unmodified †[CDKN2B- | GUGUCCTTCATGCTTUGGAU (SEQ ID NO: 21) |
| AS1]1415 | |
| Unmodified [CDKN2B- | AGAAAGAAGCAAAGAUUCAA (SEQ ID NO: 22) |
| AS1]1519 | |
| Unmodified #[CDKN2B- | CCUAGAAAGAAGCAAAGAUU (SEQ ID NO: 164) |
| AS1]1522 | |
| Unmodified [CDKN2B- | GUCAAACCTAGAAAGAAGCA (SEQ ID NO: 221) |
| AS1]1528 | |
| Unmodified #[CDKN2B- | GAUUAAAACAGATTAAUACA (SEQ ID NO: 165) |
| AS1]1773 | |
| Unmodified #[CDKN2B- | GGAUUAAAACAGATTAAUAC (SEQ ID NO: 55) |
| AS1]1774 | |
| Unmodified [CDKN2B- | AGGAUTAAAACAGATUAAUA (SEQ ID NO: 232) |
| AS1]1775 | |
| Unmodified [CDKN2B- | AGAUUATCTTCTTTTAAUUU (SEQ ID NO: 23) |
| AS1]2130 | |
| Unmodified [CDKN2B- | AAGAUTATCTTCTTTUAAUU (SEQ ID NO: 234) |
| AS1]2131 | |
| Unmodified [CDKN2B- | AAAGATTATCTTCTTUUAAU (SEQ ID NO: 241) |
| AS1]2132 | |
| Unmodified [CDKN2B- | AAAAGATTATCTTCTUUUAA (SEQ ID NO: 24) |
| AS1]2133 | |
| Unmodified [CDKN2B- | GAAAAGATTATCTTCUUUUA (SEQ ID NO: 25) |
| AS1]2134 | |
| Unmodified [CDKN2B- | UGUGAAAAGATTATCUUCUU (SEQ ID NO: 26) |
| AS1]2137 | |
| Unmodified [CDKN2B- | UUGUGAAAAGATTATCUUCU (SEQ ID NO: 229) |
| AS1]2138 | |
| Unmodified #[CDKN2B- | CUUGUGAAAAGATTAUCUUC (SEQ ID NO: 27) |
| AS1]2139 | |
| Unmodified †[CDKN2B- | GGUGGCCACAGGCAACGUCA (SEQ ID NO: 28) |
| AS1]2196 | |
| Unmodified [CDKN2B- | AAGGUGGCCACAGGCAACGU (SEQ ID NO: 464) |
| AS1]2198 | |
| Unmodified [CDKN2B- | AGGCCTCCAGTGTCTUCUCC (SEQ ID NO: 238) |
| AS1]2218 | |
| Unmodified [CDKN2B- | CAGGCCTCCAGTGTCUUCUC (SEQ ID NO: 147) |
| AS1]2219 | |
| Unmodified [CDKN2B- | GUCCCAGGCCTCCAGUGUCU (SEQ ID NO: 218) |
| AS1]2223 | |
| Unmodified #[CDKN2B- | CCAUGTCCCAGGCCTCCAGU (SEQ ID NO: 29) |
| AS1]2227 | |
| Unmodified †[CDKN2B- | UCUCCATGTCCCAGGCCUCC (SEQ ID NO: 216) |
| AS1]2230 | |
| Unmodified [CDKN2B- | GUCUCCATGTCCCAGGCCUC (SEQ ID NO: 465) |
| AS1]2231 | |
| Unmodified †[CDKN2B- | AGUCUCCATGTCCCAGGCCU (SEQ ID NO: 217) |
| AS1]2232 | |
| Unmodified #[CDKN2B- | CAGUCTCCATGTCCCAGGCC (SEQ ID NO: 240) |
| AS1]2233 | |
| Unmodified [CDKN2B- | GCAGUCTCCATGTCCCAGGC (SEQ ID NO: 30) |
| AS1]2234 | |
| Unmodified [CDKN2B- | AGCAGTCTCCATGTCCCAGG (SEQ ID NO: 31) |
| AS1]2235 | |
| Unmodified [CDKN2B- | AAGCAGTCTCCATGTCCCAG (SEQ ID NO: 231) |
| AS1]2236 | |
| Unmodified [CDKN2B- | AAAGCAGTCTCCATGUCCCA (SEQ ID NO: 224) |
| AS1]2237 | |
| Unmodified [CDKN2B-AS1]495 | AGUGGCACATACCACACCCU (SEQ ID NO: 32) |
| Unmodified †[CDKN2B- | GUGUUTTTAATTTTGUAGAG (SEQ ID NO: 220) |
| AS1]611 | |
| Unmodified [CDKN2B-AS1]613 | CAGUGTTTTTAATTTUGUAG (SEQ ID NO: 466) |
| Unmodified [CDKN2B-AS1]626 | AUUUCCACATGCCCAGUGUU (SEQ ID NO: 33) |
| Unmodified [CDKN2B-AS1]627 | UAUUUCCACATGCCCAGUGU (SEQ ID NO: 225) |
| Unmodified #[CDKN2B- | AAUUUAAAGCATGAAUAUUA (SEQ ID NO: 166) |
| AS1]645 | |
| Unmodified [CDKN2B-AS1]79 | AAAAUAAGGGGAATAGGGGA (SEQ ID NO: 467) |
| Unmodified [CDKN2B-AS1]80 | UAAAATAAGGGGAATAGGGG (SEQ ID NO: 233) |
| Unmodified [CDKN2B-AS1]831 | AUAUCTGCTGCCCACCUUCU (SEQ ID NO: 144) |
| Unmodified [CDKN2B-AS1]832 | AAUAUCTGCTGCCCACCUUC (SEQ ID NO: 236) |
| Unmodified [LINC-PINT]101 | UCCCATCCCTTCTGCUGCCA (SEQ ID NO: 235) |
| Unmodified †[LINC-PINT]102 | GUCCCATCCCTTCTGCUGCC (SEQ ID NO: 145) |
| Unmodified [LINC-PINT]103 | GGUCCCATCCCTTCTGCUGC (SEQ ID NO: 34) |
| Unmodified #[LINC-PINT]106 | UCUGGTCCCATCCCTUCUGC (SEQ ID NO: 35) |
| Unmodified †§[LINC-PINT]108 | UCUCUGGTCCCATCCCUUCU (SEQ ID NO: 143) |
| Unmodified §[LINC-PINT]109 | CUCUCTGGTCCCATCCCUUC (SEQ ID NO: 468) |
| Unmodified [LINC-PINT]110 | UCUCUCTGGTCCCATCCCUU (SEQ ID NO: 237) |
| Unmodified †[LINC-PINT]111 | UUCUCTCTGGTCCCAUCCCU (SEQ ID NO: 213) |
| Unmodified [LINC-PINT]112 | CUUCUCTCTGGTCCCAUCCC (SEQ ID NO: 154) |
| Unmodified #[LINC-PINT]113 | CCUUCTCTCTGGTCCCAUCC (SEQ ID NO: 142) |
| Unmodified [LINC-PINT]114 | CCCUUCTCTCTGGTCCCAUC (SEQ ID NO: 155) |
| Unmodified #[LINC-PINT]115 | ACCCUTCTCTCTGGTCCCAU (SEQ ID NO: 156) |
| Unmodified §[LINC-PINT]116 | CACCCTTCTCTCTGGUCCCA (SEQ ID NO: 141) |
| Unmodified [LINC-PINT]1222 | UUAGCTCCTTGCCTCGUUCC (SEQ ID NO: 226) |
| Unmodified [LINC-PINT]126 | GUCUCCTCCACACCCUUCUC (SEQ ID NO: 146) |
| Unmodified [LINC-PINT]127 | GGUCUCCTCCACACCCUUCU (SEQ ID NO: 222) |
| Unmodified †[LINC-PINT]128 | GGGUCTCCTCCACACCCUUC (SEQ ID NO: 36) |
| Unmodified [LINC-PINT]1284 | UCCCAACTCTTCTAACUCGU (SEQ ID NO: 148) |
| KL | |
| Unmodified #[LINC-PINT]1315 | GCAAGGCAGAGAAACUCCAG (SEQ ID NO: 37) |
| KL | |
| Unmodified #[LINC-PINT]148.1 | AAAUGTCCTGGCCCTCACUG (SEQ ID NO: 162) |
| Unmodified [LINC-PINT]1497.1 | GAUGGTTCCAGTCCCUCUUC (SEQ ID NO: 38) |
| Unmodified #[LINC- | UUGGCCTGTGGATGCUUUGU (SEQ ID NO: 161) |
| PINT]2673.1 | |
| Unmodified #[LINC-PINT]2690 | UUUGAAATTCAGAAGAUUUG (SEQ ID NO: 230) |
| Unmodified [LINC-PINT]2755 | CUUUATATTACAAAGCUACU (SEQ ID NO: 469) |
| Unmodified [LINC-PINT]283.1 | UGAUGATGCTTGCAGGAGGC (SEQ ID NO: 223) |
| Unmodified †[LINC-PINT]2990 | CACUGTATTTTATTACAGAA (SEQ ID NO: 470) |
| Unmodified #[LINC-PINT]384 | UGACAAAACAATAATAACAG (SEQ ID NO: 167) |
| Unmodified [LINC-PINT]412 KL | GUUCAGTCAGATCGCUGGGA (SEQ ID NO: 214) |
| Unmodified [LINC-PINT]501 KL | UGUUUCCCCGGAGAGCAAUG (SEQ ID NO: 39) |
| Unmodified †[LINC-PINT]523 | UGACATTTCGTGGCTCCUAC (SEQ ID NO: 228) |
| KL | |
| Unmodified †[LINC-PINT]524.1 | AUGACATTTCGTGGCUCCUA (SEQ ID NO: 215) |
| Unmodified [LINC-PINT]587 KL | AGCCGAACAGAAGGAGCGUC (SEQ ID NO: 40) |
| Unmodified [LINC-PINT]727.1 | GUCCGTACCTCCACCCACCG (SEQ ID NO: 219) |
| Unmodified †[LINC-PINT]83.1 | CAAGCCCCAGCGTTCCUCCG (SEQ ID NO: 239) |
| Unmodified [LINC-PINT]877 KL | CCCUAATGCTTTCCTCUCCA (SEQ ID NO: 471) |
| Unmodified #[LINC-PINT]935 | GCGUAGTTTCTCTTCCUCCC (SEQ ID NO: 41) |
| KL | |
| Unmodified [LINC-PINT]937.1 | UGGCGTAGTTTCTCTUCCUC (SEQ ID NO: 227) |
| Unmodified †§[LINC-PINT]94 | CCUUCTGCTGCCAAGCCCCA (SEQ ID NO: 472) |
| Unmodified [LINC-PINT]98 | CAUCCCTTCTGCTGCCAAGC (SEQ ID NO: 473) |
| Unmodified [LINC-PINT]99 | CCAUCCCTTCTGCTGCCAAG (SEQ ID NO: 474) |

TABLE 3
Additional oligonucleotide sequences

| | |
|---|---|
| F5-EGFR- | /52MOErT/*/i2MOErG/*/i2MOErT/*/i2MOErC/*/i2MOErC/*T*T*G*C*A*C*G |
| 1014-MOE- | *T*G*C*/i2MOErC/*/i2MOErT/*/i2MOErA/*/i2MOErA/*/32MOErG/ |
| Mut | |
| B3-CTNN2B- | /52MOErG/*/i2MOErG/*/i2MOErG/*/i2MOErA/*/i2MOErA/*A*C*G*C*T*A*T |
| 916-MOE-Mut | *G*C*A*/i2MOErA/*/i2MOErG/*/i2MOErG/*/i2MOErT/*/32MOErC/ |
| F10-MB21D1- | /52MOErG/*/i2MOErG/*/i2MOErA/*/i2MOErC/*/i2MOErC/*C*T*C*G*A*G*G |
| 616-MOE-Mut | *C*C*C*/i2MOErC/*/i2MOErA/*/i2MOErG/*/i2MOErG/*/32MOErC/ |
| A1-CTNN2B- | +C*+G*+C*T*T*T*T*C*T*C*T*C*C*+G*+G*+T |
| 311-LNA-Mut | |
| D2-CTNN2B- | +G*+C*+C*C*G*C*T*C*A*G*T*G*A*+T*+G*+T |
| 1642-LNA-Mut | |
| ShortMut1: | mG*mG*mU*mA*mU |
| ShortMut1v3: | mC*mG*mU*mU*mU |
| Short660v1: | mC*mU*mU*mC*mG |
| HPRT-2OMe- | mA*mU*mA* mG*mG*A* C*T*C* C*A*G* A*T*G* mU*mU*mU* mC*mC |
| 847 | |
| HPRT-2OMe- | mG*mG*mU* mA*mU*mA* mG*mG*A* C*T*C* C*A*G* A*T*G* |
| 847-Mut1 | mU*mU*mU* mC*mC |

TABLE 3A
Unmodified oligonucleotide sequences described in Table 3.

| Unmodified F5-EGFR-1014-MOE-Mut | TGTCCTTGCACGTGCCTAAG (SEQ ID NO: 475) |
|---|---|
| Unmodified B3-CTNN2B-916-MOE- | GGGAAACGCTATGCAAGGTC (SEQ ID NO: 476) |
| Mut | |
| Unmodified F10-MB21D1-616-MOE- | GGACCCTCGAGGCCCCAGGC (SEQ ID NO: 477) |
| Mut | |
| Unmodified A1-CTNN2B-311-LNA-Mut | CGCTTTTCTCTCCGGT (SEQ ID NO: 117) |
| Unmodified D2-CTNN2B-1642-LNA- | GCCCGCTCAGTGATGT (SEQ ID NO: 478) |
| Mut | |
| Unmodified ShortMut1: | GGUAU (SEQ ID NO: 56) |
| Unmodified ShortMut1v3: | CGUUU (SEQ ID NO: 349) |
| Unmodified Short660v1: | CUUCG (SEQ ID NO: 479) |
| Unmodified HPRT-2OMe-847 | AUAGGACTCCAGATGUUUCC (SEQ ID NO: 480) |
| Unmodified HPRT-2OMe-847-Mut1 | GGUAUAGGACTCCAGATGUUUCC (SEQ ID NO: |
| | 118) |

TABLE 4
LNA modified oligonucleotide data

| | | | TLR8 | TLR8 | TLR7 | TLR7 | TLR9 | TLR9 | cGAS |
|---|---|---|---|---|---|---|---|---|---|
| Well | Name | Sequence | 500 nM | 100 nM | 500 nM | 100 nM | 500 nM | 100 nM | 100 nM |
| A1 | CTNN2B- | +C*+G*+C*T*T*T* | 2.896 | 0.920 | 0.142 | 0.393 | 0.178 | 0.827 | 0.475 |
| | 311 LNA | T*C*T*G*T*C*T*+ | | | | | | | |
| | KL | G*+G*+T | | | | | | | |
| A10 | HPRT | +A*+C*+A*C*T*T* | 1.029 | 0.422 | 0.086 | 0.548 | 0.001 | 0.241 | 0.516 |
| | LNA-665 | C*G*T*G*G*G*G*+ | | | | | | | |
| | V1 | T*+C*+C | | | | | | | |
| A11 | HPRT | +T*+G*+A*T*G*G* | 0.263 | 0.374 | 0.153 | 0.657 | 0.153 | 0.770 | 0.650 |
| | LNA-332 | C*C*T*C*C*C*A*+ | | | | | | | |
| | V1 | T*+C*+T | | | | | | | |
| A12 | HPRT | +A*+T*+G*G*C*C* | 1.356 | 0.369 | 0.117 | 0.550 | 0.244 | 0.511 | 0.475 |
| | LNA-330 | T*C*C*C*A*T*C*+ | | | | | | | |
| | V1 | T*+C*+C | | | | | | | |
| A2 | CTNN2B- | +C*+C*+A*C*T*T* | 1.213 | 0.684 | 0.470 | 0.765 | 0.111 | 1.112 | 0.532 |
| | 1575 LNA | G*G*C*A*G*A*C*+ | | | | | | | |
| | V1 | C*+A*+T | | | | | | | |
| A3 | EGFR- | +G*+T*+G*A*T*C* | 1.806 | 0.583 | 0.060 | 0.438 | 0.181 | 1.001 | 0.661 |
| | 2790 LNA | T*T*G*A*C*A*T*+ | | | | | | | |
| | KL | G*+C*+T | | | | | | | |
| A4 | EGFR- | +A*+G*+G*C*C*C* | 0.967 | 0.507 | 0.164 | 0.737 | 0.211 | 0.576 | 0.629 |
| | 1225 LNA | T*T*C*G*C*A*C*+ | | | | | | | |
| | V1 | T*+T*+C | | | | | | | |
| A5 | CAMSAP1- | +A*+C*+C*A*C*G* | 4.259 | 0.933 | 0.532 | 1.078 | 0.162 | 0.842 | 0.638 |
| | 1635 LNA | T*C*T*G*C*T*C*+ | | | | | | | |
| | KL | T*+A*+A | | | | | | | |
| A6 | CAMSAP1- | +G*+C*+A*C*A*C* | 1.091 | 0.319 | 0.221 | 0.657 | 0.147 | 0.683 | 0.383 |
| | 4915 LNA | T*T*C*G*T*A*C*+ | | | | | | | |
| | V1 | C*+C*+A | | | | | | | |
| A7 | MB21D1- | +A*+A*+C*C*C*C* | 8.654 | 1.871 | 0.426 | 0.771 | 0.076 | 0.586 | 0.621 |
| | 689 LNA | T*T*T*C*A*C*C*+ | | | | | | | |
| | KL | A*+T*+C | | | | | | | |
| A8 | MB21D1- | +G*+A*+G*G*T*C* | 0.642 | 0.574 | 0.067 | 0.793 | 0.224 | 0.897 | 0.508 |
| | 525 LNA | T*T*G*G*C*T*T*+ | | | | | | | |
| | V1 | C*+G*+T | | | | | | | |
| A9 | HPRT | +C*+C*+A*G*C*A* | 1.499 | 0.412 | 0.942 | 1.101 | 0.149 | 0.736 | 0.552 |
| | LNA-388 | G*G*T*C*A*G*C*+ | | | | | | | |
| | KL | A*+A*+A | | | | | | | |
| B1 | CTNN2B- | +G*+A*+A*A*G*G* | 4.110 | 1.156 | 0.101 | 0.166 | 0.606 | 0.748 | 0.473 |
| | 916 LNA | T*T*A*T*G*C*A*+ | | | | | | | |
| | KL | A*+G*+G | | | | | | | |
| B10 | HPRT | +G*+T*+G*A*T*G* | 1.112 | 0.517 | 0.139 | 0.665 | 0.694 | 0.890 | 0.724 |
| | LNA-333 | G*C*C*T*C*C*C*+ | | | | | | | |
| | V1 | A*+T*+C | | | | | | | |
| B11 | HPRT | +A*+T*+G*T*G*A* | 1.840 | 0.637 | 0.128 | 0.499 | 0.451 | 0.776 | 0.606 |
| | LNA-335 | T*G*G*C*C*T*C*+ | | | | | | | |
| | V1 | C*+C*+A | | | | | | | |
| B12 | HPRT | +T*+C*+G*T*G*G* | 2.966 | 0.778 | 0.318 | 0.540 | 0.462 | 0.461 | 0.729 |
| | LNA-660 | G*G*T*C*C*T*T*+ | | | | | | | |
| | V1 | T*+T*+C | | | | | | | |
| B2 | CTNN2B- | +T*+G*+T*C*T*G* | 0.871 | 0.600 | 0.278 | 0.495 | 0.648 | 1.320 | 0.617 |
| | 808 LNA | G*A*A*G*C*T*T*+ | | | | | | | |
| | V1 | C*+C*+T | | | | | | | |
| B3 | EGFR- | +A*+G*+T*G*T*T* | 0.599 | 0.427 | 0.115 | 0.559 | 0.540 | 0.982 | 0.692 |
| | 3652 LNA | G*A*G*A*T*A*C*+ | | | | | | | |
| | KL | T*+C*+G | | | | | | | |
| B4 | EGFR- | +G*+T*+C*C*T*T* | 0.936 | 0.570 | 0.251 | 0.471 | 0.722 | 1.310 | 0.645 |
| | 1015 LNA | G*C*A*C*G*T*G*+ | | | | | | | |
| | V1 | G*+C*+T | | | | | | | |
| B5 | CAMSAP1- | +T*+A*+C*T*T*T* | 1.585 | 0.417 | 0.451 | 0.738 | 0.356 | 0.915 | 0.638 |
| | 1739 LNA | C*C*G*A*G*G*T*+ | | | | | | | |
| | KL | G*+T*+C | | | | | | | |
| B6 | CAMSAP1- | +G*+G*+C*T*C*G* | 0.706 | 0.374 | 0.186 | 0.569 | 0.350 | 0.714 | 0.761 |
| | 5594 LNA | G*C*T*T*G*C*C*+ | | | | | | | |
| | V1 | T*+A*+C | | | | | | | |
| B7 | MB21D1- | +G*+C*+A*C*T*T* | 0.864 | 0.495 | 0.168 | 0.713 | 0.144 | 0.673 | 0.797 |
| | 719 LNA | C*A*G*T*C*T*G*+ | | | | | | | |
| | KL | A*+G*+C | | | | | | | |
| B8 | MB21D1- | +G*+G*+T*C*T*T* | 1.386 | 0.616 | 0.042 | 0.481 | 0.417 | 0.658 | 0.648 |
| | 523 LNA | G*G*C*T*T*C*G*+ | | | | | | | |
| | V1 | T*+G*+G | | | | | | | |
| B9 | HPRT | +G*+G*+T*C*A*T* | 1.381 | 0.500 | 0.049 | 0.580 | 0.604 | 0.438 | 0.751 |
| | LNA-475 | T*A*C*A*A*T*A*+ | | | | | | | |
| | KL | G*+C*+T | | | | | | | |
| C1 | CTNN2B- | +G*+A*+T*A*G*C* | 2.863 | 0.821 | 0.049 | 0.434 | 0.560 | 0.804 | 0.436 |
| | 1294 LNA | A*C*C*T*T*C*A*+ | | | | | | | |
| | KL | G*+C*+A | | | | | | | |
| C10 | HPRT | +C*+A*+A*C*A*C* | 0.690 | 0.591 | 0.476 | 0.663 | 0.395 | 0.733 | 0.675 |
| | LNA-667 | T*T*C*G*T*G*G*+ | | | | | | | |
| | V1 | G*+G*+T | | | | | | | |
| C11 | HPRT | +T*+G*+T*G*A*T* | 1.596 | 0.582 | 0.090 | 0.315 | 0.431 | 0.818 | 0.557 |
| | LNA-334 | G*G*C*C*T*C*C*+ | | | | | | | |
| | V1 | C*+A*+T | | | | | | | |
| C12 | HPRT | +T*+C*+C*A*A*C* | 0.491 | 0.413 | 0.249 | 0.318 | 0.473 | 0.623 | 0.539 |
| | LNA-669 | A*C*T*T*C*G*T*+ | | | | | | | |
| | V1 | G*+G*+G | | | | | | | |
| C2 | CTNN2B- | +C*+C*+A*T*C*C* | 1.169 | 0.675 | 0.644 | 0.992 | 0.476 | 1.383 | 0.533 |
| | 2545 LNA | A*T*G*A*G*G*T*+ | | | | | | | |
| | V1 | C*+C*+T | | | | | | | |
| C3 | EGFR- | +G*+T*+A*T*C*G* | 1.781 | 0.637 | 0.113 | 0.562 | 0.647 | 1.429 | 0.609 |
| | 3908 LNA | A*A*A*G*A*G*T*+ | | | | | | | |
| | KL | C*+T*+G | | | | | | | |
| C4 | EGFR- | +G*+C*+C*A*T*C* | 0.681 | 0.440 | 0.123 | 0.671 | 0.589 | 0.772 | 0.688 |
| | 2874 LNA | C*A*C*T*T*G*A*+ | | | | | | | |
| | V1 | T*+A*+G | | | | | | | |
| C5 | CAMSAP1- | +G*+G*+T*T*A*C* | 1.848 | 0.804 | 0.140 | 0.438 | 0.400 | 0.943 | 0.661 |
| | 4295 LNA | G*G*C*T*C*A*G*+ | | | | | | | |
| | KL | T*+A*+T | | | | | | | |
| C6 | CAMSAP1- | +T*+G*+C*A*C*A* | 0.370 | 0.411 | 0.299 | 0.806 | 0.244 | 0.283 | 0.572 |
| | 4916 LNA | C*T*T*C*G*T*A*+ | | | | | | | |
| | V1 | C*+C*+C | | | | | | | |
| C7 | MB21D1- | +T*+C*+G*T*A*G* | 1.583 | 0.557 | 0.198 | 0.380 | 0.224 | 0.987 | 0.610 |
| | 1181 LNA | T*T*G*C*T*T*C*+ | | | | | | | |
| | KL | C*+T*+A | | | | | | | |
| C8 | MB21D1- | +G*+T*+C*T*T*G* | 2.137 | 0.678 | 0.056 | 0.652 | 0.392 | 0.745 | 0.620 |
| | 522 LNA | G*C*T*T*C*G*T*+ | | | | | | | |
| | V1 | G*+G*+A | | | | | | | |
| C9 | HPRT | +G*+T*+C*C*C*C* | 0.304 | 0.335 | 0.123 | 0.557 | 0.326 | 0.387 | 0.606 |
| | LNA-489 | T*G*T*T*G*A*C*+ | | | | | | | |
| | KL | T*+G*+G | | | | | | | |
| D1 | CTNN2B- | +C*+C*+A*A*G*A* | 2.920 | 1.015 | 0.697 | 0.882 | 0.669 | 1.424 | 0.356 |
| | 2341 LNA | T*C*A*G*C*A*G*+ | | | | | | | |
| | KL | T*+C*+T | | | | | | | |
| D10 | HPRT | +T*+G*+G*C*C*T* | 1.127 | 0.495 | 0.217 | 0.877 | 0.277 | 0.743 | 0.494 |
| | LNA-329 | C*C*C*A*T*C*T*+ | | | | | | | |
| | V1 | C*+C*+T | | | | | | | |
| D11 | HPRT | +T*+T*+C*G*T*G* | 3.144 | 0.621 | 0.178 | 0.487 | 0.523 | 0.963 | 0.520 |
| | LNA-661 | G*G*G*T*C*C*T*+ | | | | | | | |
| | V1 | T*+T*+T | | | | | | | |
| D12 | HPRT | +G*+T*+T*C*A*G* | 4.129 | 1.069 | 0.114 | 0.456 | 0.382 | 1.081 | 0.563 |
| | LNA-292 | T*C*C*T*G*T*C*+ | | | | | | | |
| | V1 | C*+A*+T | | | | | | | |
| D2 | CTNN2B- | +G*+C*+A*G*G*C* | 1.440 | 0.638 | 0.189 | 0.676 | 0.228 | 1.036 | 0.458 |
| | 1642 LNA | T*C*A*G*T*G*A*+ | | | | | | | |
| | V1 | T*+G*+T | | | | | | | |
| D3 | EGFR- | +G*+G*+C*T*G*G* | 0.577 | 0.562 | 0.041 | 0.417 | 0.492 | 1.051 | 0.592 |
| | 4705 LNA | A*A*T*C*C*G*A*+ | | | | | | | |
| | KL | G*+T*+T | | | | | | | |
| D4 | EGFR- | +G*+C*+A*T*C*C* | 0.738 | 0.607 | 0.085 | 0.555 | 0.375 | 0.556 | 0.602 |
| | 3267 LNA | A*C*C*A*C*G*T*+ | | | | | | | |
| | V1 | C*+G*+T | | | | | | | |
| D5 | CAMSAP1- | +T*+T*+T*T*G*G* | 1.412 | 0.542 | 0.305 | 0.564 | 0.169 | 0.985 | 0.590 |
| | 4697 LNA | C*T*G*G*G*A*T*+ | | | | | | | |
| | KL | C*+A*+A | | | | | | | |
| D6 | CAMSAP1- | +G*+C*+C*A*T*G* | 0.363 | 0.446 | 0.175 | 0.638 | 0.147 | 0.879 | 0.599 |
| | 2584 LNA | C*T*G*G*C*T*C*+ | | | | | | | |
| | V1 | G*+G*+A | | | | | | | |
| D7 | MB21D1- | +G*+C*+C*A*T*G* | 0.431 | 0.430 | 0.021 | 0.816 | 0.070 | 0.163 | 0.639 |
| | 1248 LNA | T*T*T*C*T*T*C*+T | | | | | | | |
| | KL | *+T*+G | | | | | | | |
| D8 | MB21D1- | +C*+G*+G*C*C*T* | 0.219 | 0.402 | 0.139 | 0.594 | 0.116 | 0.218 | 0.617 |
| | 168 LNA | C*G*G*A*A*G*C*+ | | | | | | | |
| | V1 | T*+C*+T | | | | | | | |
| D9 | HPRT | +T*+C*+C*A*C*A* | 0.893 | 0.474 | 0.533 | 0.863 | 0.308 | 0.851 | 0.601 |
| | LNA-551 | A*T*C*A*A*G*A*+ | | | | | | | |
| | KL | C*+A*+T | | | | | | | |
| E1 | CTNN2B- | +C*+T*+T*G*A*A* | 3.889 | 0.960 | 0.044 | 0.363 | 0.483 | 0.899 | 0.373 |
| | 3648 LNA | G*C*A*T*C*G*T*+ | | | | | | | |
| | KL | A*+T*+C | | | | | | | |
| E10 | HPRT | +A*+A*+C*A*C*T* | 0.882 | 0.496 | 0.445 | 0.899 | 0.475 | 1.104 | 0.598 |
| | LNA-666 | T*C*G*T*G*G*G*+ | | | | | | | |
| | V1 | G*+T*+C | | | | | | | |
| E11 | HPRT | +C*+T*+T*C*G*T* | 4.366 | 1.097 | 0.233 | 0.685 | 0.240 | 0.557 | 0.556 |
| | LNA-662 | G*G*G*G*T*C*C*+ | | | | | | | |
| | V1 | T*+T*+T | | | | | | | |
| E12 | HPRT | +A*+G*+G*A*C*T* | 1.034 | 0.278 | 0.293 | 0.437 | 0.511 | 0.816 | 0.632 |
| | LNA pos | C*C*A*G*A*T*G*+ | | | | | | | |
| | cont | T*+T*+T | | | | | | | |
| E2 | CTNN2B- | +A*+T*+C*T*G*G* | 2.628 | 0.808 | 0.279 | 0.663 | 0.603 | 1.248 | 0.502 |
| | 2516 LNA | C*A*G*C*C*C*A*+ | | | | | | | |
| | V1 | T*+C*+A | | | | | | | |
| E3 | EGFR- | +A*+G*+A*T*T*T* | 0.770 | 0.401 | 0.021 | 0.438 | 0.438 | 0.848 | 0.625 |
| | 4885 LNA | C*A*G*A*G*C*A*+ | | | | | | | |
| | KL | G*+C*+T | | | | | | | |
| E4 | EGFR- | +C*+C*+T*T*G*C* | 2.414 | 1.315 | 0.281 | 0.617 | 0.519 | 0.814 | 0.559 |
| | 1013 LNA | A*C*G*T*G*G*C*+ | | | | | | | |
| | V1 | T*+T*+C | | | | | | | |
| E5 | CAMSAP1- | +A*+A*+A*G*G*A* | 2.663 | 0.882 | 0.267 | 0.513 | 0.185 | 0.780 | 0.675 |
| | 6273 LNA | C*T*G*A*G*G*A*+ | | | | | | | |
| | KL | A*+A*+G | | | | | | | |
| E6 | CAMSAP1- | +T*+T*+C*A*G*G* | 0.764 | 0.479 | 0.181 | 0.642 | 0.494 | 1.202 | 0.709 |
| | 2709 LNA | C*G*C*T*G*C*C*+ | | | | | | | |
| | V1 | T*+T*+G | | | | | | | |
| E7 | MB21D1- | +T*+G*+A*C*T*G* | 3.210 | 0.973 | 0.150 | 0.802 | 0.290 | 1.245 | 0.747 |
| | 1483 LNA | T*C*T*T*G*A*G*+ | | | | | | | |
| | KL | G*+G*+T | | | | | | | |
| E8 | MB21D1- | +C*+T*+T*G*G*C* | 0.594 | 0.458 | 0.098 | 0.806 | 0.371 | 0.596 | 0.666 |
| | 520 LNA | T*T*C*G*T*G*G*+ | | | | | | | |
| | V1 | A*+G*+C | | | | | | | |
| E9 | HPRT | +A*+A*+T*C*C*A* | 1.900 | 0.591 | 0.433 | 0.727 | 0.529 | 1.050 | 0.649 |
| | LNA-692 | A*C*A*A*A*G*T*+ | | | | | | | |
| | KL | C*+T*+G | | | | | | | |
| F1 | CTNN2B- | +G*+T*+G*T*C*T* | 2.057 | 0.923 | 0.067 | 0.355 | 0.613 | 0.493 | 0.482 |
| | 809 LNA | G*G*A*A*G*C*T*+ | | | | | | | |
| | V1 | T*+C*+C | | | | | | | |
| F10 | HPRT | +C*+A*+C*T*T*C* | 1.446 | 0.425 | 0.240 | 0.868 | 0.074 | 0.215 | 0.562 |
| | LNA-664 | G*T*G*G*G*G*T*+ | | | | | | | |
| | V1 | C*+C*+T | | | | | | | |
| F11 | HPRT | +G*+A*+T*G*G*C* | 0.186 | 0.278 | 0.119 | 0.558 | 0.476 | 1.033 | 0.632 |
| | LNA-331 | C*T*C*C*C*A*T*+ | | | | | | | |
| | V1 | C*+T*+C | | | | | | | |
| F12 | NC1 LNA | +C*+G*+T*A*T*T* | 0.716 | 0.396 | 0.226 | 0.754 | 0.558 | 0.933 | 0.443 |
| | PS 3-10-3 | A*T*A*G*C*C*G*+ | | | | | | | |
| | | A*+T*+T | | | | | | | |
| F2 | CTNN2B- | +C*+T*+G*C*A*G* | 0.936 | 0.478 | 0.053 | 0.467 | 0.263 | 0.358 | 0.613 |
| | 2136 LNA | C*T*T*C*C*T*T*+ | | | | | | | |
| | V1 | G*+T*+C | | | | | | | |
| F3 | EGFR- | +T*+C*+C*T*T*G* | 0.601 | 0.491 | 0.482 | 0.812 | 1.027 | 1.152 | 0.591 |
| | 1014 LNA | C*A*C*G*T*G*G*+ | | | | | | | |
| | V1 | C*+T*+T | | | | | | | |
| F4 | EGFR- | +T*+G*+T*C*C*T* | 1.158 | 0.636 | 0.199 | 0.502 | 0.853 | 1.020 | 0.678 |
| | 1016 LNA | T*G*C*A*C*G*T*+ | | | | | | | |
| | V1 | G*+G*+C | | | | | | | |
| F5 | CAMSAP1- | +T*+C*+T*T*C*A* | 1.393 | 0.595 | 0.511 | 0.820 | 0.445 | 0.921 | 0.548 |
| | 2112 LNA | T*C*G*G*C*C*C*+ | | | | | | | |
| | V1 | T*+G*+C | | | | | | | |
| F6 | CAMSAP1- | +G*+T*+C*T*C*T* | 2.402 | 0.764 | 0.084 | 0.477 | 0.272 | 0.935 | 0.627 |
| | 2456 LNA | G*G*A*G*C*T*T*+ | | | | | | | |
| | V1 | C*+C*+T | | | | | | | |
| F7 | MB21D1- | +T*+C*+T*T*G*G* | 0.197 | 0.541 | 0.288 | 1.033 | 0.595 | 1.251 | 0.693 |
| | 521 LNA | C*T*T*C*G*T*G*+ | | | | | | | |
| | V1 | G*+A*+G | | | | | | | |
| F8 | MB21D1- | +A*+G*+C*T*T*C* | 0.865 | 0.331 | 0.111 | 0.554 | 0.085 | 0.114 | 0.648 |
| | 616 LNA | G*A*G*G*C*C*C*+ | | | | | | | |
| | V1 | C*+A*+G | | | | | | | |
| F9 | HPRT | +G*+T*+C*A*A*G* | 2.301 | 0.543 | 0.142 | 0.650 | 0.387 | 0.844 | 0.636 |
| | LNA-732 | G*G*C*A*T*A*T*+ | | | | | | | |
| | KL | C*+C*+T | | | | | | | |
| G1 | CTNN2B- | +T*+C*+C*A*T*A* | 1.334 | 0.595 | 0.494 | 1.056 | 0.951 | 1.075 | 0.589 |
| | 2446 LNA | C*C*C*A*A*G*G*+ | | | | | | | |
| | V1 | C*+A*+T | | | | | | | |
| G10 | HPRT | +C*+C*+A*A*C*A* | 0.835 | 0.535 | 0.446 | 0.808 | 0.610 | 1.065 | 0.842 |
| | LNA-668 | C*T*T*C*G*T*G*+ | | | | | | | |
| | V1 | G*+G*+G | | | | | | | |
| G11 | HPRT | +A*+C*+G*T*T*C* | 0.493 | 0.356 | 0.136 | 0.601 | 0.198 | 0.427 | 0.880 |
| | LNA-294 | A*G*T*C*C*T*G*+ | | | | | | | |
| | V1 | T*+C*+C | | | | | | | |
| G12 | NC5 LNA | +G*+A*+C*T*A*T* | 3.376 | 0.777 | 0.098 | 0.282 | 0.403 | 0.784 | 0.991 |
| | PS 3-10-3 | A*C*G*C*G*C*A*+ | | | | | | | |
| | | A*+T*+A | | | | | | | |
| G2 | CTNN2B- | +T*+G*+G*T*G*G* | 0.604 | 0.464 | 0.393 | 0.957 | 1.117 | 1.136 | 0.830 |
| | 2479 LNA | C*C*A*C*C*C*A*+ | | | | | | | |
| | V1 | T*+C*+T | | | | | | | |
| G3 | EGFR- | +C*+A*+T*C*C*A* | 2.417 | 0.778 | 0.353 | 0.741 | 0.620 | 0.722 | 0.902 |
| | 3266 LNA | C*C*A*C*G*T*C*+ | | | | | | | |
| | V1 | G*+T*+C | | | | | | | |
| G4 | EGFR-727 | +G*+T*+C*C*C*G* | 0.745 | 0.585 | 0.311 | 0.843 | 1.172 | 0.871 | 0.908 |
| | LNA VI | C*C*A*C*T*G*G*+ | | | | | | | |
| | | A*+T*+G | | | | | | | |
| G5 | CAMSAP1- | +G*+T*+C*T*T*C* | 2.086 | 0.664 | 0.119 | 0.641 | 0.617 | 0.797 | 0.809 |
| | 2113 LNA | A*T*C*G*G*C*C*+ | | | | | | | |
| | V1 | C*+T*+G | | | | | | | |
| G6 | CAMSAP1- | +C*+C*+A*C*A*T* | 0.232 | 0.291 | 0.363 | 0.789 | 0.337 | 0.338 | 0.999 |
| | 2906 LNA | C*C*T*G*T*G*G*+ | | | | | | | |
| | V1 | C*+T*+C | | | | | | | |
| G7 | MB21D1- | +A*+G*+G*T*C*T* | 1.953 | 0.596 | 0.050 | 0.457 | 0.383 | 0.975 | 0.947 |
| | 524 LNA | T*G*G*C*T*T*C*+ | | | | | | | |
| | V1 | G*+T*+G | | | | | | | |
| G8 | MB21D1- | +T*+G*+G*T*C*C* | 1.454 | 0.559 | 0.545 | 1.008 | 0.587 | 1.018 | 0.935 |
| | 697 LNA | A*C*A*A*C*C*C*+ | | | | | | | |
| | V1 | C*+T*+T | | | | | | | |
| G9 | HPRT | +T*+C*+C*G*C*C* | 0.725 | 0.541 | 0.543 | 0.860 | 0.599 | 0.876 | 0.860 |
| | LNA-1027 | C*A*A*A*G*G*G*+ | | | | | | | |
| | KL | A*+A*+C | | | | | | | |
| H1 | CTNN2B- | +C*+C*+C*A*C*T* | 1.865 | 0.899 | 0.639 | 1.024 | 0.656 | 0.381 | |
| | 1576 LNA | T*G*G*C*A*G*A*+ | | | | | | | |
| | V1 | C*+C*+A | | | | | | | |
| H10 | HPRT | +A*+C*+T*T*C*G* | 3.672 | 0.820 | 0.736 | 0.804 | 0.377 | 0.377 | 0.937 |
| | LNA-663 | T*G*G*G*G*T*C*+ | | | | | | | |
| | V1 | C*+T*+T | | | | | | | |
| H11 | HPRT | +C*+C*+T*C*C*C* | 6.759 | 1.469 | 0.843 | 0.758 | 0.727 | 1.010 | |
| | LNA-326 | A*T*C*T*C*C*T*+ | | | | | | | |
| | V1 | T*+C*+A | | | | | | | |
| H3 | EGFR- | +G*+G*+C*A*C*T* | 0.862 | 0.652 | 0.668 | 0.774 | 0.788 | 1.092 | |
| | 2859 LNA | T*T*G*C*C*T*C*+ | | | | | | | |
| | V1 | C*+T*+T | | | | | | | |
| H5 | CAMSAP1- | +T*+G*+C*T*C*C* | 4.771 | 1.097 | 0.595 | 0.751 | 0.535 | 0.548 | |
| | 1850 LNA | T*C*G*G*T*C*T*+ | | | | | | | |
| | V1 | C*+C*+C | | | | | | | |
| H7 | MB21D1- | +G*+G*+A*G*G*T* | 0.882 | 0.575 | 0.119 | 0.503 | 0.674 | 0.912 | 1.061 |
| | 526 LNA | C*T*T*G*G*C*T*+ | | | | | | | |
| | V1 | T*+C*+G | | | | | | | |
| H9 | HPRT | +T*+G*+A*C*A*A* | 1.756 | 0.633 | 0.430 | 1.040 | 0.574 | 0.819 | 0.887 |
| | LNA-1353 | A*G*A*T*T*C*A*+ | | | | | | | |
| | KL | C*+T*+G | | | | | | | |

TABLE 4A
Unmodified oligonucleotide sequences described in Table 4.

| Name | Sequence |
|---|---|
| Unmodified CTNN2B-311 LNA KL | CGCTTTTCTGTCTGGT (SEQ ID NO: 105) |
| Unmodified HPRT LNA-665 V1 | ACACTTCGTGGGGTCC (SEQ ID NO: 110) |
| Unmodified HPRT LNA-332 V1 | TGATGGCCTCCCATCT (SEQ ID NO: 481) |
| Unmodified HPRT LNA-330 V1 | ATGGCCTCCCATCTCC (SEQ ID NO: 104) |
| Unmodified CTNN2B-1575 LNA V1 | CCACTTGGCAGACCAT (SEQ ID NO: 112) |
| Unmodified EGFR-2790 LNA KL | GTGATCTTGACATGCT (SEQ ID NO: 312) |
| Unmodified EGFR-1225 LNA V1 | AGGCCCTTCGCACTTC (SEQ ID NO: 201) |
| Unmodified CAMSAP1-1635 LNA KL | ACCACGTCTGCTCTAA (SEQ ID NO: 482) |
| Unmodified CAMSAP1-4915 LNA V1 | GCACACTTCGTACCCA (SEQ ID NO: 99) |
| Unmodified MB21D1-689 LNA KL | AACCCCTTTCACCATC (SEQ ID NO: 483) |
| Unmodified MB21D1-525 LNA V1 | GAGGTCTTGGCTTCGT (SEQ ID NO: 109) |
| Unmodified HPRT LNA-388 KL | CCAGCAGGTCAGCAAA (SEQ ID NO: 116) |
| Unmodified CTNN2B-916 LNA KL | GAAAGGTTATGCAAGG (SEQ ID NO: 103) |
| Unmodified HPRT LNA-333 V1 | GTGATGGCCTCCCATC (SEQ ID NO: 484) |
| Unmodified HPRT LNA-335 V1 | ATGTGATGGCCTCCCA (SEQ ID NO: 485) |
| Unmodified HPRT LNA-660 V1 | TCGTGGGGTCCTTTTC (SEQ ID NO: 197) |
| Unmodified CTNN2B-808 LNA V1 | TGTCTGGAAGCTTCCT (SEQ ID NO: 486) |
| Unmodified EGFR-3652 LNA KL | AGTGTTGAGATACTCG (SEQ ID NO: 487) |
| Unmodified EGFR-1015 LNA V1 | GTCCTTGCACGTGGCT (SEQ ID NO: 317) |
| Unmodified CAMSAP1-1739 LNA KL | TACTTTCCGAGGTGTC (SEQ ID NO: 488) |
| Unmodified CAMSAP1-5594 LNA V1 | GGCTCGGCTTGCCTAC (SEQ ID NO: 489) |
| Unmodified MB21D1-719 LNA KL | GCACTTCAGTCTGAGC (SEQ ID NO: 490) |
| Unmodified MB21D1-523 LNA V1 | GGTCTTGGCTTCGTGG (SEQ ID NO: 319) |
| Unmodified HPRT LNA-475 KL | GGTCATTACAATAGCT (SEQ ID NO: 196) |
| Unmodified CTNN2B-1294 LNA KL | GATAGCACCTTCAGCA (SEQ ID NO: 100) |
| Unmodified HPRT LNA-667 V1 | CAACACTTCGTGGGGT (SEQ ID NO: 491) |
| Unmodified HPRT LNA-334 V1 | TGTGATGGCCTCCCAT (SEQ ID NO: 308) |
| Unmodified HPRT LNA-669 V1 | TCCAACACTTCGTGGG (SEQ ID NO: 114) |
| Unmodified CTNN2B-2545 LNA V1 | CCATCCATGAGGTCCT (SEQ ID NO: 113) |
| Unmodified EGFR-3908 LNA KL | GTATCGAAAGAGTCTG (SEQ ID NO: 492) |
| Unmodified EGFR-2874 LNA V1 | GCCATCCACTTGATAG (SEQ ID NO: 493) |
| Unmodified CAMSAP1-4295 LNA KL | GGTTACGGCTCAGTAT (SEQ ID NO: 314) |
| Unmodified CAMSAP1-4916 LNA V1 | TGCACACTTCGTACCC (SEQ ID NO: 189) |
| Unmodified MB21D1-1181 LNA KL | TCGTAGTTGCTTCCTA (SEQ ID NO: 309) |
| Unmodified MB21D1-522 LNA V1 | GTCTTGGCTTCGTGGA (SEQ ID NO: 494) |
| Unmodified HPRT LNA-489 KL | GTCCCCTGTTGACTGG (SEQ ID NO: 194) |
| Unmodified CTNN2B-2341 LNA KL | CCAAGATCAGCAGTCT (SEQ ID NO: 97) |
| Unmodified HPRT LNA-329 V1 | TGGCCTCCCATCTCCT (SEQ ID NO: 107) |
| Unmodified HPRT LNA-661 V1 | TTCGTGGGGTCCTTTT (SEQ ID NO: 111) |
| Unmodified HPRT LNA-292 V1 | GTTCAGTCCTGTCCAT (SEQ ID NO: 315) |
| Unmodified CTNN2B-1642 LNA V1 | GCAGGCTCAGTGATGT (SEQ ID NO: 102) |
| Unmodified EGFR-4705 LNA KL | GGCTGGAATCCGAGTT (SEQ ID NO: 310) |
| Unmodified EGFR-3267 LNA V1 | GCATCCACCACGTCGT (SEQ ID NO: 199) |
| Unmodified CAMSAP1-4697 LNA KL | TTTTGGCTGGGATCAA (SEQ ID NO: 495) |
| Unmodified CAMSAP1-2584 LNA V1 | GCCATGCTGGCTCGGA (SEQ ID NO: 496) |
| Unmodified MB21D1-1248 LNA KL | GCCATGTTTCTTCTTG (SEQ ID NO: 186) |
| Unmodified MB21D1-168 LNA V1 | CGGCCTCGGAAGCTCT (SEQ ID NO: 188) |
| Unmodified HPRT LNA-551 KL | TCCACAATCAAGACAT (SEQ ID NO: 497) |
| Unmodified CTNN2B-3648 LNA KL | CTTGAAGCATCGTATC (SEQ ID NO: 98) |
| Unmodified HPRT LNA-666 V1 | AACACTTCGTGGGGTC (SEQ ID NO: 498) |
| Unmodified HPRT LNA-662 V1 | CTTCGTGGGGTCCTTT (SEQ ID NO: 200) |
| Unmodified HPRT LNA pos cont | AGGACTCCAGATGTTT (SEQ ID NO: 311) |
| Unmodified CTNN2B-2516 LNA V1 | ATCTGGCAGCCCATCA (SEQ ID NO: 108) |
| Unmodified EGFR-4885 LNA KL | AGATTTCAGAGCAGCT (SEQ ID NO: 313) |
| Unmodified EGFR-1013 LNA V1 | CCTTGCACGTGGCTTC (SEQ ID NO: 499) |
| Unmodified CAMSAP1-6273 LNA KL | AAAGGACTGAGGAAAG (SEQ ID NO: 500) |
| Unmodified CAMSAP1-2709 LNA V1 | TTCAGGCGCTGCCTTG (SEQ ID NO: 501) |
| Unmodified MB21D1-1483 LNA KL | TGACTGTCTTGAGGGT (SEQ ID NO: 502) |
| Unmodified MB21D1-520 LNA V1 | CTTGGCTTCGTGGAGC (SEQ ID NO: 503) |
| Unmodified HPRT LNA-692 KL | AATCCAACAAAGTCTG (SEQ ID NO: 504) |
| Unmodified CTNN2B-809 LNA V1 | GTGTCTGGAAGCTTCC (SEQ ID NO: 106) |
| Unmodified HPRT LNA-664 V1 | CACTTCGTGGGGTCCT (SEQ ID NO: 187) |
| Unmodified HPRT LNA-331 V1 | GATGGCCTCCCATCTC (SEQ ID NO: 505) |
| Unmodified NC1 LNA PS 3-10-3 | CGTATTATAGCCGATT (SEQ ID NO: 101) |
| Unmodified CTNN2B-2136 LNA V1 | CTGCAGCTTCCTTGTC (SEQ ID NO: 191) |
| Unmodified EGFR-1014 LNA V1 | TCCTTGCACGTGGCTT (SEQ ID NO: 506) |
| Unmodified EGFR-1016 LNA V1 | TGTCCTTGCACGTGGC (SEQ ID NO: 507) |
| Unmodified CAMSAP1-2112 LNA V1 | TCTTCATCGGCCCTGC (SEQ ID NO: 115) |
| Unmodified CAMSAP1-2456 LNA V1 | GTCTCTGGAGCTTCCT (SEQ ID NO: 318) |
| Unmodified MB21D1-521 LNA V1 | TCTTGGCTTCGTGGAG (SEQ ID NO: 508) |
| Unmodified MB21D1-616 LNA V1 | AGCTTCGAGGCCCCAG (SEQ ID NO: 185) |
| Unmodified HPRT LNA-732 KL | GTCAAGGGCATATCCT (SEQ ID NO: 509) |
| Unmodified CTNN2B-2446 LNA V1 | TCCATACCCAAGGCAT (SEQ ID NO: 510) |
| Unmodified HPRT LNA-668 V1 | CCAACACTTCGTGGGG (SEQ ID NO: 511) |
| Unmodified HPRT LNA-294 V1 | ACGTTCAGTCCTGTCC (SEQ ID NO: 195) |
| Unmodified NC5 LNA PS 3-10-3 | GACTATACGCGCAATA (SEQ ID NO: 307) |
| Unmodified CTNN2B-2479 LNA V1 | TGGTGGCCACCCATCT (SEQ ID NO: 512) |
| Unmodified EGFR-3266 LNA V1 | CATCCACCACGTCGTC (SEQ ID NO: 513) |
| Unmodified EGFR-727 LNA V1 | GTCCCGCCACTGGATG (SEQ ID NO: 514) |
| Unmodified CAMSAP1-2113 LNA V1 | GTCTTCATCGGCCCTG (SEQ ID NO: 515) |
| Unmodified CAMSAP1-2906 LNA V1 | CCACATCCTGTGGCTC (SEQ ID NO: 190) |
| Unmodified MB21D1-524 LNA V1 | AGGTCTTGGCTTCGTG (SEQ ID NO: 316) |
| Unmodified MB21D1-697 LNA V1 | TGGTCCACAACCCCTT (SEQ ID NO: 516) |
| Unmodified HPRT LNA-1027 KL | TCCGCCCAAAGGGAAC (SEQ ID NO: 517) |
| Unmodified CTNN2B-1576 LNA V1 | CCCACTTGGCAGACCA (SEQ ID NO: 193) |
| Unmodified HPRT LNA-663 V1 | ACTTCGTGGGGTCCTT (SEQ ID NO: 192) |
| Unmodified HPRT LNA-326 V1 | CCTCCCATCTCCTTCA (SEQ ID NO: 518) |
| Unmodified EGFR-2859 LNA V1 | GGCACTTTGCCTCCTT (SEQ ID NO: 519) |
| Unmodified CAMSAP1-1850 LNA V1 | TGCTCCTCGGTCTCCC (SEQ ID NO: 198) |
| Unmodified MB21D1-526 LNA V1 | GGAGGTCTTGGCTTCG (SEQ ID NO: 520) |
| Unmodified HPRT LNA-1353 KL | TGACAAAGATTCACTG (SEQ ID NO: 521) |

TABLE 5
MOE modified oligonucleotide data

| | | | TLR8 | TLR8 | TLR7 | TLR7 | TLR9 | TLR9 | cGAS |
|---|---|---|---|---|---|---|---|---|---|
| | | | 500 | 100 | 500 | 100 | 500 | 100 | 100 |
| Well | ASO | Sequence | nM | nM | nM | nM | nM | nM | nM |
| A1 | HPRT- | /52MOErA/*/i2MOErT/*/i2MOErG/* | 2.118 | 1.752 | 0.224 | 0.426 | 0.953 | 0.968 | 0.574 |
| | 489 | /i2MOErT/*/i2MOErC/*C*C*C*T*G*T* | | | | | | | |
| | MOE | T*G*A*C*/i2MOErT/*/i2MOErG/* | | | | | | | |
| | | /i2MOErG/*/i2MOErT/*/32MOErC/ | | | | | | | |
| A10 | MB21 | /52MOErC/*/i2MOErG/*/i2MOErG/* | 3.257 | 2.197 | 0.138 | 0.505 | 0.556 | 0.938 | 0.111 |
| | D1- | /i2MOErA/*/i2MOErG/*G*T*C*T*T*G | | | | | | | |
| | 525 | *G*C*T*T*/i2MOErC/*/i2MOErG/*/ | | | | | | | |
| | MOE | i2MOErT/*/i2MOErG/*/32MOErG/ | | | | | | | |
| A2 | HPRT- | /52MOErC/*/i2MOErC/*/i2MOErA/* | 1.185 | 1.223 | 0.334 | 0.856 | 0.600 | 0.612 | 0.557 |
| | 666 | /i2MOErA/*/i2MOErC/*A*C*T*T*C*G* | | | | | | | |
| | MOE | T*G*G*G*/i2MOErG/*/i2MOErT/* | | | | | | | |
| | | /i2MOErC/*/i2MOErC/*/32MOErT/ | | | | | | | |
| A3 | CTN | /52MOErG/*/i2MOErC/*/i2MOErC/* | 0.369 | 0.968 | 0.268 | 0.567 | 1.018 | 0.684 | 0.401 |
| | N2B- | /i2MOErG/*/i2MOErC/*T*T*T*T*C*T* | | | | | | | |
| | 311 | G*T*C*T*/i2MOErG/*/i2MOErG/* | | | | | | | |
| | MOE | /i2MOErT/*/i2MOErT/*/32MOErC/ | | | | | | | |
| A4 | CTN | /52MOErA/*/i2MOErC/*/i2MOErC/* | 3.985 | 2.822 | 0.336 | 0.473 | 0.556 | 0.725 | 0.571 |
| | N2B- | /i2MOErC/*/i2MOErA/*C*T*T*G*G*C* | | | | | | | |
| | 1575 | A*G*A*C*/i2MOErC/*/i2MOErA/* | | | | | | | |
| | MOE | /i2MOErT/*/i2MOErC/*/32MOErA/ | | | | | | | |
| A5 | EGFR- | /52MOErC/*/i2MOErT/*/i2MOErG/* | 1.387 | 1.308 | 0.206 | 0.449 | 0.298 | 0.506 | 0.180 |
| | 2790 | /i2MOErT/*/i2MOErG/*A*T*C*T*T*G* | | | | | | | |
| | MOE | A*C*A*T*/i2MOErG/*/i2MOErC/* | | | | | | | |
| | | /i2MOErT/*/i2MOErG/*/32MOErC/ | | | | | | | |
| A6 | EGFR- | /52MOErC/*/i2MOErA/*/i2MOErA/* | 1.236 | 1.115 | 0.336 | 0.587 | 0.712 | 0.624 | 0.490 |
| | 1225 | /i2MOErG/*/i2MOErG/*C*C*C*T*T*C | | | | | | | |
| | MOE | *G*C*A*C*/i2MOErT/*/i2MOErT/* | | | | | | | |
| | | /i2MOErC/*/i2MOErT/*/32MOErT/ | | | | | | | |
| A7 | CAM | /52MOErG/*/i2MOErA/*/i2MOErA/* | 3.827 | 2.787 | 0.291 | 0.597 | 0.466 | 0.588 | 0.518 |
| | SAP1- | /i2MOErC/*/i2MOErC/*A*C*G*T*C*T | | | | | | | |
| | 1635 | *G*C*T*C*/i2MOErT/*/i2MOErA/* | | | | | | | |
| | MOE | /i2MOErA/*/i2MOErC/*/32MOErA/ | | | | | | | |
| A8 | CAM | /52MOErT/*/i2MOErT/*/i2MOErG/* | 1.972 | 1.555 | 0.233 | 0.418 | 0.524 | 0.894 | 0.392 |
| | SAP1- | /i2MOErC/*/i2MOErA/*C*A*C*T*T*C* | | | | | | | |
| | 4915 | G*T*A*C*/i2MOErC/*/i2MOErC/* | | | | | | | |
| | MOE | /i2MOErA/*/i2MOErA/*/32MOErA/ | | | | | | | |
| A9 | MB21 | /52MOErA/*/i2MOErC/*/i2MOErA/* | 14.808 | 8.841 | 0.533 | 0.593 | 0.499 | 0.785 | 0.658 |
| | D1- | /i2MOErA/*/i2MOErC/*C*C*C*T*T*T* | | | | | | | |
| | 689 | C*A*C*C*/i2MOErA/*/i2MOErT/* | | | | | | | |
| | MOE | /i2MOErC/*/i2MOErC/*/32MOErC/ | | | | | | | |
| B1 | HPRT- | /52MOErT/*/i2MOErA/*/i2MOErG/* | 2.087 | 1.457 | 0.191 | 0.423 | 0.462 | 0.787 | 0.848 |
| | 732 | /i2MOErT/*/i2MOErC/*A*A*G*G*G*C* | | | | | | | |
| | MOE | A*T*A*T*/i2MOErC/*/i2MOErC/* | | | | | | | |
| | | /i2MOErT/*/i2MOErA/*/32MOErC/ | | | | | | | |
| B10 | MB21 | /52MOErG/*/i2MOErA/*/i2MOErG/* | 2.164 | 1.822 | 0.095 | 0.419 | 0.650 | 0.888 | 0.380 |
| | D1- | /i2MOErG/*/i2MOErT/*C*T*T*G*G*C | | | | | | | |
| | 523 | *T*T*C*G*/i2MOErT/*/i2MOErG/* | | | | | | | |
| | MOE | /i2MOErG/*/i2MOErA/*/32MOErG/ | | | | | | | |
| B2 | HPRT- | /52MOErA/*/i2MOErA/*/i2MOErC/* | 3.167 | 2.725 | 0.226 | 0.406 | 0.402 | 0.426 | 0.615 |
| | 664 | /i2MOErA/*/i2MOErC/*T*T*C*G*T*G | | | | | | | |
| | MOE | *G*G*G*T*/i2MOErC/*/i2MOErC/* | | | | | | | |
| | | /i2MOErT/*/i2MOErT/*/32MOErT/ | | | | | | | |
| B3 | CTN | /52MOErG/*/i2MOErG/*/i2MOErG/* | 3.500 | 3.117 | 0.163 | 0.599 | 0.505 | 0.521 | 0.156 |
| | N2B- | /i2MOErA/*/i2MOErA/*A*G*G*T*T*A | | | | | | | |
| | 916 | *T*G*C*A*/i2MOErA/*/i2MOErG/* | | | | | | | |
| | MOE | /i2MOErG/*/i2MOErT/*/32MOErC/ | | | | | | | |
| B4 | CTN | /52MOErC/*/i2MOErG/*/i2MOErT/* | 1.222 | 1.476 | 0.350 | 0.541 | 0.259 | 0.568 | 0.426 |
| | N2B- | /i2MOErG/*/i2MOErT/*C*T*G*G*A*A* | | | | | | | |
| | 808 | G*C*T*T*/i2MOErC/*/i2MOErC/* | | | | | | | |
| | MOE | /i2MOErT/*/i2MOErT/*/32MOErT/ | | | | | | | |
| B5 | EGFR- | /52MOErA/*/i2MOErC/*/i2MOErA/* | 8.261 | 5.681 | 0.129 | 0.420 | 0.194 | 0.436 | 0.297 |
| | 3652 | /i2MOErG/*/i2MOErT/*G*T*T*G*A*G | | | | | | | |
| | MOE | *A*T*A*C*/i2MOErT/*/i2MOErC/* | | | | | | | |
| | | /i2MOErG/*/i2MOErG/*/32MOErG/ | | | | | | | |
| B6 | EGFR- | /52MOErG/*/i2MOErT/*/i2MOErG/* | 1.274 | 1.383 | 0.212 | 0.377 | 0.641 | 0.784 | 0.384 |
| | 1015 | /i2MOErT/*/i2MOErC/*C*T*T*G*C*A* | | | | | | | |
| | MOE | C*G*T*G*/i2MOErG/*/i2MOErC/* | | | | | | | |
| | | /i2MOErT/*/i2MOErT/*/32MOErC/ | | | | | | | |
| B7 | CAM | /52MOErC/*/i2MOErT/*/i2MOErT/* | 4.827 | 3.892 | 0.336 | 0.588 | 0.531 | 0.647 | 0.473 |
| | SAP1- | /i2MOErA/*/i2MOErC/*T*T*T*C*C*G* | | | | | | | |
| | 1739 | A*G*G*T*/i2MOErG/*/i2MOErT/* | | | | | | | |
| | MOE | /i2MOErC/*/i2MOErC/*/32MOErA/ | | | | | | | |
| B8 | CAM | /52MOErT/*/i2MOErT/*/i2MOErG/* | 0.813 | 1.096 | 0.204 | 0.523 | 0.445 | 0.705 | 0.295 |
| | SAP1- | /i2MOErG/*/i2MOErC/*T*C*G*G*C*T* | | | | | | | |
| | 5594 | T*G*C*C*/i2MOErT/*/i2MOErA/* | | | | | | | |
| | MOE | /i2MOErC/*/i2MOErT/*/32MOErT/ | | | | | | | |
| B9 | MB21 | /52MOErT/*/i2MOErC/*/i2MOErG/* | 2.158 | 1.697 | 0.241 | 0.574 | 0.266 | 0.521 | 0.305 |
| | D1- | /i2MOErC/*/i2MOErA/*C*T*T*C*A*G* | | | | | | | |
| | 719 | T*C*T*G*/i2MOErA/*/i2MOErG/* | | | | | | | |
| | MOE | /i2MOErC/*/i2MOErA/*/32MOErG/ | | | | | | | |
| C1 | HPRT- | /52MOErA/*/i2MOErA/*/i2MOErT/* | 4.659 | 2.685 | 0.521 | 0.691 | 0.591 | 0.591 | 0.494 |
| | 1027 | /i2MOErC/*/i2MOErC/*G*C*C*C*A*A* | | | | | | | |
| | MOE | A*G*G*G*/i2MOErA/*/i2MOErA/* | | | | | | | |
| | | /i2MOErC/*/i2MOErT/*/32MOErG/ | | | | | | | |
| C10 | MB21 | /52MOErA/*/i2MOErG/*/i2MOErG/* | 5.064 | 3.657 | 0.150 | 0.547 | 0.280 | 0.568 | 0.118 |
| | D1- | /i2MOErT/*/i2MOErC/*T*T*G*G*C*T* | | | | | | | |
| | 522 | T*C*G*T*/i2MOErG/*/i2MOErG/* | | | | | | | |
| | MOE | /i2MOErA/*/i2MOErG/*/32MOErC/ | | | | | | | |
| C2 | HPRT- | /52MOErA/*/i2MOErT/*/i2MOErC/* | 1.519 | 1.437 | 0.611 | 0.758 | 0.618 | 0.287 | 0.744 |
| | 668 | /i2MOErC/*/i2MOErA/*A*C*A*C*T*T* | | | | | | | |
| | MOE | C*G*T*G*/i2MOErG/*/i2MOErG/* | | | | | | | |
| | | /i2MOErG/*/i2MOErT/*/32MOErC/ | | | | | | | |
| C3 | CTN | /52MOErC/*/i2MOErA/*/i2MOErG/* | 2.356 | 1.773 | 0.254 | 0.497 | 0.484 | 0.583 | 0.388 |
| | N2B- | /i2MOErA/*/i2MOErT/*A*G*C*A*C*C | | | | | | | |
| | 1294 | *T*T*C*A*/i2MOErG/*/i2MOErC/* | | | | | | | |
| | MOE | /i2MOErA/*/i2MOErC/*/32MOErT/ | | | | | | | |
| C4 | CTN | /52MOErG/*/i2MOErC/*/i2MOErC/* | 0.541 | 0.899 | 0.124 | 0.340 | 0.175 | 0.324 | 0.592 |
| | N2B- | /i2MOErC/*/i2MOErA/*T*C*C*A*T*G* | | | | | | | |
| | 2545 | A*G*G*T*/i2MOErC/*/i2MOErC/* | | | | | | | |
| | MOE | /i2MOErT/*/i2MOErG/*/32MOErG/ | | | | | | | |
| C5 | EGFR- | /52MOErG/*/i2MOErG/*/i2MOErG/* | 4.073 | 3.138 | 0.079 | 0.350 | 0.203 | 0.349 | 0.427 |
| | 3908 | /i2MOErT/*/i2MOErA/*T*C*G*A*A*A | | | | | | | |
| | MOE | *G*A*G*T*/i2MOErC/*/i2MOErT/* | | | | | | | |
| | | /i2MOErG/*/i2MOErG/*/32MOErA/ | | | | | | | |
| C6 | EGFR- | /52MOErA/*/i2MOErT/*/i2MOErG/* | 2.025 | 2.028 | 0.390 | 0.668 | 0.589 | 0.712 | 0.639 |
| | 2874 | /i2MOErC/*/i2MOErC/*A*T*C*C*A*C* | | | | | | | |
| | MOE | T*T*G*A*/i2MOErT/*/i2MOErA/* | | | | | | | |
| | | /i2MOErG/*/i2MOErG/*/32MOErC/ | | | | | | | |
| C7 | CAM | /52MOErT/*/i2MOErG/*/i2MOErG/* | 1.538 | 1.610 | 0.167 | 0.427 | 0.228 | 0.478 | 0.432 |
| | SAP1- | /i2MOErG/*/i2MOErT/*T*A*C*G*G*C* | | | | | | | |
| | 4295 | T*C*A*G*/i2MOErT/*/i2MOErA/* | | | | | | | |
| | MOE | /i2MOErT/*/i2MOErG/*/32MOErG/ | | | | | | | |
| C8 | CAM | /52MOErT/*/i2MOErT/*/i2MOErT/* | 2.199 | 1.877 | 0.118 | 0.296 | 0.514 | 0.735 | 0.335 |
| | SAP1- | /i2MOErG/*/i2MOErC/*A*C*A*C*T*T* | | | | | | | |
| | 4916 | C*G*T*A*/i2MOErC/*/i2MOErC/* | | | | | | | |
| | MOE | /i2MOErC/*/i2MOErA/*/32MOErA/ | | | | | | | |
| C9 | MB21 | /52MOErA/*/i2MOErG/*/i2MOErT/* | 5.889 | 3.091 | 0.228 | 0.494 | 0.341 | 0.527 | 0.261 |
| | D1- | /i2MOErC/*/i2MOErG/*T*A*G*T*T*G* | | | | | | | |
| | 1181 | C*T*T*C*/i2MOErC/*/i2MOErT/* | | | | | | | |
| | MOE | /i2MOErA/*/i2MOErA/*/32MOErC/ | | | | | | | |
| D1 | HPRT- | /52MOErG/*/i2MOErC/*/i2MOErT/* | 3.067 | 1.475 | 0.190 | 0.375 | 0.654 | 0.653 | 0.335 |
| | 1353 | /i2MOErG/*/i2MOErA/*C*A*A*A*G*A* | | | | | | | |
| | MOE | T*T*C*A*/i2MOErC/*/i2MOErT/* | | | | | | | |
| | | /i2MOErG/*/i2MOErG/*/32MOErT/ | | | | | | | |
| D10 | MB21 | /52MOErT/*/i2MOErC/*/i2MOErC/* | 0.781 | 0.816 | 0.166 | 0.440 | 0.000 | 0.242 | 0.250 |
| | D1- | /i2MOErG/*/i2MOErG/*C*C*T*C*G*G* | | | | | | | |
| | 168 | A*A*G*C*/i2MOErT/*/i2MOErC/* | | | | | | | |
| | MOE | /i2MOET/*/i2MOErC/*/32MOErT/ | | | | | | | |
| D2 | HPRT- | /52MOErA/*/i2MOErC/*/i2MOErA/* | 6.320 | 4.778 | 0.235 | 0.403 | 0.226 | 0.114 | 0.623 |
| | 663 | /i2MOErC/*/i2MOErT/*T*C*G*T*G*G | | | | | | | |
| | MOE | *G*G*T*C*/i2MOErC/*/i2MOErT/* | | | | | | | |
| | | /i2MOErT/*/i2MOErT/*/32MOErT/ | | | | | | | |
| D3 | CTN | /52MOErG/*/i2MOErT/*/i2MOErC/* | 3.364 | 3.021 | 0.120 | 0.298 | 0.535 | 0.430 | 0.705 |
| | N2B- | /i2MOErC/*/i2MOErA/*A*G*A*T*C*A* | | | | | | | |
| | 2341 | G*C*A*G*/i2MOErT/*/i2MOErC/* | | | | | | | |
| | MOE | /i2MOErT/*/i2MOErC/*/32MOErA/ | | | | | | | |
| D4 | CTN | /52MOErT/*/i2MOErG/*/i2MOErG/* | 0.973 | 1.074 | 0.167 | 0.415 | 0.479 | 0.722 | 0.605 |
| | N2B- | /i2MOErC/*/i2MOErA/*G*G*C*T*C*A* | | | | | | | |
| | 1642 | G*T*G*A*/i2MOErT/*/i2MOErG/* | | | | | | | |
| | MOE | /i2MOErT/*/i2MOErC/*/32MOErT/ | | | | | | | |
| D5 | EGFR- | /52MOErT/*/i2MOErG/*/i2MOErG/* | 2.058 | 1.452 | 0.095 | 0.270 | 0.241 | 0.443 | 0.433 |
| | 4705 | /i2MOErG/*/i2MOErC/*T*G*G*A*A*T* | | | | | | | |
| | MOE | C*C*G*A*/i2MOErG/*/i2MOErT/* | | | | | | | |
| | | /i2MOErT/*/i2MOErA/*/32MOErT/ | | | | | | | |
| D6 | EGFR- | /52MOErC/*/i2MOErG/*/i2MOErG/* | 0.903 | 1.249 | 0.202 | 0.485 | 0.373 | 0.449 | 0.378 |
| | 3267 | /i2MOErC/*/i2MOErA/*T*C*C*A*C*C | | | | | | | |
| | MOE | *A*C*G*T*/i2MOErC/*/i2MOErG/* | | | | | | | |
| | | /i2MOErT/*/i2MOErC/*/32MOErC/ | | | | | | | |
| D7 | CAM | /52MOErG/*/i2MOErG/*/i2MOErT/* | 6.332 | 5.496 | 0.191 | 0.346 | 0.200 | 0.381 | 0.375 |
| | SAP1- | /i2MOErT/*/i2MOErT/*T*G*G*C*T*G* | | | | | | | |
| | 4697 | G*G*A*T*/i2MOErC/*/i2MOErA/* | | | | | | | |
| | MOE | /i2MOErA/*/i2MOErG/*/32MOErT/ | | | | | | | |
| D8 | CAM | /52MOErT/*/i2MOErT/*/i2MOErG/* | 0.572 | 0.881 | 0.138 | 0.445 | 0.534 | 0.516 | 0.518 |
| | SAP1- | /i2MOErC/*/i2MOErC/*A*T*G*C*T*G* | | | | | | | |
| | 2584 | G*C*T*C*/i2MOErG/*/i2MOErG/* | | | | | | | |
| | MOE | /i2MOErA/*/i2MOErC/*/32MOErT/ | | | | | | | |
| D9 | MB21 | /52MOErC/*/i2MOErC/*/i2MOErG/* | 2.713 | 1.909 | 0.139 | 0.402 | 0.251 | 0.481 | 0.361 |
| | DI- | /i2MOErC/*/i2MOErC/*A*T*G*T*T*T* | | | | | | | |
| | 1248 | C*T*T*C*/i2MOErT/*/i2MOErT/* | | | | | | | |
| | MOE | /i2MOErG/*/i2MOErG/*/32MOErA/ | | | | | | | |
| E1 | HPRT- | /52MOErC/*/i2MOErA/*/i2MOErA/* | 1.639 | 1.320 | 0.285 | 0.487 | 0.619 | 0.858 | 0.686 |
| | 665 | /i2MOErC/*/i2MOErA/*C*T*T*C*G*T* | | | | | | | |
| | MOE | G*G*G*G*/i2MOErT/*/i2MOErC/* | | | | | | | |
| | | /i2MOErC/*/i2MOErT/*/32MOErT/ | | | | | | | |
| E10 | MB21 | /52MOErG/*/i2MOErT/*/i2MOErC/* | 3.536 | 2.383 | 0.095 | 0.372 | 0.599 | 0.709 | 0.291 |
| | D1- | /i2MOErT/*/i2MOErT/*G*G*C*T*T*C* | | | | | | | |
| | 520 | G*T*G*G*/i2MOErA/*/i2MOErG/* | | | | | | | |
| | MOE | /i2MOErC/*/i2MOErA/*/32MOErG/ | | | | | | | |
| E2 | HPRT- | /52MOErT/*/i2MOErG/*/i2MOErT/* | 0.938 | 0.940 | 0.238 | 0.312 | 0.782 | 0.674 | 0.438 |
| | 332 | /i2MOErG/*/i2MOErA/*T*G*G*C*C*T* | | | | | | | |
| | MOE | C*C*C*A*/i2MOErT/*/i2MOErC/* | | | | | | | |
| | | /i2MOErT/*/i2MOErC/*/32MOErC/ | | | | | | | |
| E3 | CTN | /52MOErC/*/i2MOErT/*/i2MOErC/* | 2.200 | 1.650 | 0.341 | 0.609 | 0.472 | 0.721 | 0.791 |
| | N2B- | /i2MOErT/*/i2MOErT/*G*A*A*G*C*A* | | | | | | | |
| | 3648 | T*C*G*T*/i2MOErA/*/i2MOErT/* | | | | | | | |
| | MOE | /i2MOErC/*/i2MOErA/*/32MOErC/ | | | | | | | |
| E4 | CTN | /52MOErA/*/i2MOErG/*/i2MOErA/* | 4.465 | 3.069 | 0.201 | 0.500 | 0.268 | 0.607 | 0.677 |
| | N2B- | /i2MOErT/*/i2MOErC/*T*G*G*C*A*G | | | | | | | |
| | 2516 | *C*C*C*A*/i2MOErT/*/i2MOErC/* | | | | | | | |
| | MOE | /i2MOErA/*/i2MOErA/*/32MOErC/ | | | | | | | |
| E5 | EGFR- | /52MOErG/*/i2MOErG/*/i2MOErA/* | 4.308 | 3.122 | 0.088 | 0.330 | 0.381 | 0.541 | 0.466 |
| | 4885 | /i2MOErG/*/i2MOErA/*T*T*T*C*A*G | | | | | | | |
| | MOE | *A*G*C*A*/i2MOErG/*/i2MOErC/* | | | | | | | |
| | | /i2MOErT/*/i2MOErT/*/32MOErC/ | | | | | | | |
| E6 | EGFR- | /52MOErG/*/i2MOErT/*/i2MOErC/* | 1.593 | 1.900 | 0.088 | 0.216 | 0.602 | 0.682 | 0.374 |
| | 1013 | /i2MOErC/*/i2MOErT/*T*G*C*A*C*G* | | | | | | | |
| | MOE | T*G*G*C*/i2MOErT/*/i2MOErT/* | | | | | | | |
| | | /i2MOErC/*/i2MOErG/*/32MOErT/ | | | | | | | |
| E7 | CAM | /52MOErT/*/i2MOErC/*/i2MOErA/* | 3.406 | 3.896 | 0.189 | 0.397 | 0.040 | 0.265 | 0.634 |
| | SAP1- | /i2MOErA/*/i2MOErA/*G*G*A*C*T*G* | | | | | | | |
| | 6273 | A*G*G*A*/i2MOErA/*/i2MOErA/* | | | | | | | |
| | MOE | /i2MOErG/*/i2MOErG/*/32MOErG/ | | | | | | | |
| E8 | CAM | /52MOErG/*/i2MOErC/*/i2MOErT/* | 0.620 | 0.985 | 0.196 | 0.527 | 0.824 | 0.833 | 0.510 |
| | SAP1- | /i2MOErT/*/i2MOErC/*A*G*G*C*G*C* | | | | | | | |
| | 2709 | T*G*C*C*/i2MOErT/*/i2MOErT/* | | | | | | | |
| | MOE | /i2MOErG/*/i2MOErC/*/32MOErC/ | | | | | | | |
| E9 | MB21 | /52MOErA/*/i2MOErC/*/i2MOErT/* | 3.185 | 2.454 | 0.299 | 0.627 | 0.291 | 0.424 | 0.221 |
| | D1- | /i2MOErG/*/i2MOErA/*C*T*G*T*C*T* | | | | | | | |
| | 1483 | T*G*A*G*/i2MOErG/*/i2MOErG/* | | | | | | | |
| | MOE | /i2MOErT/*/i2MOErT/*/32MOErC/ | | | | | | | |
| F1 | HPRT- | /52MOErA/*/i2MOErT/*/i2MOErG/* | 1.429 | 0.939 | 0.345 | 0.643 | 0.918 | 0.900 | 0.439 |
| | 333 | /i2MOErT/*/i2MOErG/*A*T*G*G*C*C* | | | | | | | |
| | MOE | T*C*C*C*/i2MOErA/*/i2MOErT/* | | | | | | | |
| | | /i2MOErC/*/i2MOErT/*/32MOErC/ | | | | | | | |
| F10 | MB21 | /52MOErG/*/i2MOErG/*/i2MOErA/* | 0.380 | 0.760 | 0.179 | 0.392 | 0.168 | 0.588 | 0.117 |
| | D1- | /i2MOErG/*/i2MOErC/*T*T*C*G*A*G | | | | | | | |
| | 616 | *G*C*C*C*/i2MOErC/*/i2MOErA/* | | | | | | | |
| | MOE | /i2MOErG/*/i2MOErG/*/32MOErC/ | | | | | | | |
| F2 | HPRT- | /52MOErC/*/i2MOErA/*/i2MOErA/* | 1.677 | 1.518 | 0.279 | 0.451 | 0.412 | 0.614 | 0.362 |
| | 335 | /i2MOErT/*/i2MOErG/*T*G*A*T*G*G | | | | | | | |
| | MOE | *C*C*T*C*/i2MOErC/*/i2MOErC/* | | | | | | | |
| | | /i2MOErA/*/i2MOErT/*/32MOErC/ | | | | | | | |
| F3 | CTN | /52MOErG/*/i2MOErC/*/i2MOErG/* | 0.494 | 1.179 | 0.134 | 0.415 | 0.229 | 0.263 | 0.241 |
| | N2B- | /i2MOErT/*/i2MOErG/*T*C*T*G*G*A | | | | | | | |
| | 809 | *A*G*C*T*/i2MOErT/*/i2MOErC/* | | | | | | | |
| | MOE | /i2MOErC/*/i2MOErT/*/32MOErT/ | | | | | | | |
| F4 | CTN | /52MOErT/*/i2MOErT/*/i2MOErC/* | 2.408 | 2.049 | 0.059 | 0.183 | 0.467 | 0.746 | 0.821 |
| | N2B- | /i2MOErT/*/i2MOErG/*C*A*G*C*T*T* | | | | | | | |
| | 2136 | C*C*T*T*/i2MOErG/*/i2MOErT/* | | | | | | | |
| | MOE | /i2MOErC/*/i2MOErC/*/32MOErT/ | | | | | | | |
| F5 | EGFR- | /52MOErT/*/i2MOErG/*/i2MOErT/* | 1.433 | 1.187 | 0.057 | 0.029 | 0.623 | 0.764 | 0.515 |
| | 1014 | /i2MOErC/*/i2MOErC/*T*T*G*C*A*C* | | | | | | | |
| | MOE | G*T*G*G*/i2MOErC/*/i2MOErT/* | | | | | | | |
| | | /i2MOErT/*/i2MOErC/*/32MOErG/ | | | | | | | |
| F6 | EGFR- | /52MOErG/*/i2MOErG/*/i2MOErT/* | 1.019 | 1.532 | 0.131 | 0.358 | 0.714 | 1.064 | 0.315 |
| | 1016 | /i2MOErG/*/i2MOErT/*C*C*T*T*G*C* | | | | | | | |
| | MOE | A*C*G*T*/i2MOErG/*/i2MOErG/* | | | | | | | |
| | | /i2MOErC/*/i2MOErT/*/32MOErT/ | | | | | | | |
| F7 | CAM | /52MOErT/*/i2MOErG/*/i2MOErT/* | 1.178 | 1.331 | 0.185 | 0.396 | 0.480 | 0.697 | 0.557 |
| | SAP1- | /i2MOErC/*/i2MOErT/*T*C*A*T*C*G* | | | | | | | |
| | 2112 | G*C*C*C*/i2MOErT/*/i2MOErG/* | | | | | | | |
| | MOE | /i2MOErC/*/i2MOErC/*/32MOErT/ | | | | | | | |
| F8 | CAM | /52MOErG/*/i2MOErA/*/i2MOErG/* | 0.683 | 0.849 | 0.170 | 0.469 | 0.127 | 0.594 | 0.250 |
| | SAP1- | /i2MOErT/*/i2MOErC/*T*C*T*G*G*A | | | | | | | |
| | 2456 | *G*C*T*T*/i2MOErC/*/i2MOErC/* | | | | | | | |
| | MOE | /i2MOErT/*/i2MOErC/*/32MOErT/ | | | | | | | |
| F9 | MB21 | /52MOErG/*/i2MOErG/*/i2MOErT/* | 4.596 | 3.386 | 0.171 | 0.460 | 0.243 | 0.518 | 0.256 |
| | DI- | /i2MOErC/*/i2MOErT/*T*G*G*C*T*T* | | | | | | | |
| | 521 | C*G*T*G*/i2MOErG/*/i2MOErA/* | | | | | | | |
| | MOE | /i2MOErG/*/i2MOErC/*/32MOErA/ | | | | | | | |
| G1 | HPRT- | /52MOErT/*/i2MOErC/*/i2MOErC/* | 1.432 | 1.142 | 0.578 | 0.929 | 0.616 | 0.792 | 0.799 |
| | 667 | /i2MOErA/*/i2MOErA/*C*A*C*T*T*C* | | | | | | | |
| | MOE | G*T*G*G*/i2MOErG/*/i2MOErG/* | | | | | | | |
| | | /i2MOErT/*/i2MOErC/*/32MOErC/ | | | | | | | |
| G10 | MB21 | /52MOErG/*/i2MOErG/*/i2MOErT/* | 3.700 | 2.121 | 0.097 | 0.620 | 0.071 | 0.456 | 0.292 |
| | D1- | /i2MOErG/*/i2MOErG/*T*C*C*A*C*A* | | | | | | | |
| | 697 | A*C*C*C*/i2MOErC/*/i2MOErT/* | | | | | | | |
| | MOE | /i2MOErT/*/i2MOErT/*/32MOErC/ | | | | | | | |
| G3 | CTN | /52MOErG/*/i2MOErG/*/i2MOErT/* | 2.107 | 1.901 | 0.096 | 0.364 | 0.453 | 0.687 | 0.531 |
| | N2B- | /i2MOErC/*/i2MOErC/*A*T*A*C*C*C* | | | | | | | |
| | 2446 | A*A*G*G*/i2MOErC/*/i2MOErA/* | | | | | | | |
| | MOE | /i2MOErT/*/i2MOErC/*/32MOErC/ | | | | | | | |
| G4 | CTN | /52MOErG/*/i2MOErG/*/i2MOErT/* | 0.874 | 1.491 | 0.192 | 0.480 | 0.317 | 0.761 | 0.456 |
| | N2B- | /i2MOErG/*/i2MOErG/*T*G*G*C*C*A* | | | | | | | |
| | 2479 | C*C*C*A*/i2MOErT/*/i2MOErC/* | | | | | | | |
| | MOE | /i2MOErT/*/i2MOErC/*/32MOErA/ | | | | | | | |
| G5 | EGFR- | /52MOErG/*/i2MOErG/*/i2MOErC/* | 1.566 | 1.619 | 0.097 | 0.216 | 0.481 | 0.742 | 0.478 |
| | 3266 | /i2MOErA/*/i2MOErT/*C*C*A*C*C*A | | | | | | | |
| | MOE | *C*G*T*C*/i2MOErG/*/i2MOErT/* | | | | | | | |
| | | /i2MOErC/*/i2MOErC/*/32MOErA/ | | | | | | | |
| G6 | EGFR- | /52MOErA/*/i2MOErT/*/i2MOErG/* | 1.708 | 2.029 | 0.171 | 0.397 | 0.423 | 0.801 | 0.549 |
| | 727 | /i2MOErT/*/i2MOErC/*C*C*G*C*C*A* | | | | | | | |
| | MOE | C*T*G*G*/i2MOErA/*/i2MOErT/* | | | | | | | |
| | | /i2MOErG/*/i2MOErC/*/32MOErT/ | | | | | | | |
| G7 | CAM | /52MOErG/*/i2MOErT/*/i2MOErG/* | 1.093 | 1.247 | 0.132 | 0.371 | 0.201 | 0.611 | 0.606 |
| | SAP1- | /i2MOErT/*/i2MOErC/*T*T*C*A*T*C* | | | | | | | |
| | 2113 | G*G*C*C*/i2MOErC/*/i2MOErT/* | | | | | | | |
| | MOE | /i2MOErG/*/i2MOErC/*/32MOErC/ | | | | | | | |
| G8 | CAM | /52MOErG/*/i2MOErT/*/i2MOErC/* | 0.493 | 0.919 | 0.119 | 0.299 | 0.254 | 0.559 | 0.455 |
| | SAP1- | /i2MOErC/*/i2MOErA/*C*A*T*C*C*T* | | | | | | | |
| | 2906 | G*T*G*G*/i2MOErC/*/i2MOErT/* | | | | | | | |
| | MOE | /i2MOErC/*/i2MOErG/*/32MOErT/ | | | | | | | |
| G9 | MB21 | /52MOErG/*/i2MOErG/*/i2MOErA/* | 5.288 | 3.976 | 0.091 | 0.434 | 0.342 | 0.466 | 0.450 |
| | D1- | /i2MOErG/*/i2MOErG/*T*C*T*T*G*G | | | | | | | |
| | 524 | *C*T*T*C*/i2MOErG/*/i2MOErT/* | | | | | | | |
| | MOE | /i2MOErG/*/i2MOErG/*/32MOErA/ | | | | | | | |
| H1 | HPRT- | /52MOErG/*/i2MOErA/*/i2MOErT/* | 1.698 | 0.979 | 0.336 | 0.678 | 1.268 | 0.662 | 0.687 |
| | 329 | /i2MOErG/*/i2MOErG/*C*C*T*C*C*C* | | | | | | | |
| | MOE | A*T*C*T*/i2MOErC/*/i2MOErC/* | | | | | | | |
| | | /i2MOErT/*/i2MOErT/*/32MOErC/ | | | | | | | |
| H10 | NC5 | /52MOErG/*/i2MOErC/*/i2MOErG/* | 0.883 | 1.067 | 0.294 | 0.948 | 0.035 | 0.395 | 0.720 |
| | 2′MOE | /i2MOErA/*/i2MOErC/*T*A*T*A*C*G | | | | | | | |
| | | *C*G*C*A*/i2MOErA/*/i2MOErT/* | | | | | | | |
| | | /i2MOErA/*/i2MOErT/*/32MOErG/ | | | | | | | |
| H3 | CTN | /52MOErC/*/i2MOErA/*/i2MOErC/* | 1.502 | 1.440 | 0.227 | 0.600 | 0.677 | 0.665 | 0.658 |
| | N2B- | /i2MOErC/*/i2MOErC/*A*C*T*T*G*G* | | | | | | | |
| | 1576 | C*A*G*A*/i2MOErC/*/i2MOErC/* | | | | | | | |
| | MOE | /i2MOErA/*/i2MOErT/*/32MOErC/ | | | | | | | |
| H5 | EGFR- | /52MOErT/*/i2MOErA/*/i2MOErG/* | 0.884 | 0.773 | 0.205 | 0.455 | 0.914 | 0.808 | 0.378 |
| | 2859 | /i2MOErG/*/i2MOErC/*A*C*T*T*T*G* | | | | | | | |
| | MOE | C*C*T*C*/i2MOErC/*/i2MOErT/* | | | | | | | |
| | | /i2MOErT/*/i2MOErC/*/32MOErT/ | | | | | | | |
| H7 | CAM | /52MOErG/*/i2MOErA/*/i2MOErT/* | 1.954 | 1.392 | 0.199 | 0.453 | 0.196 | 0.792 | 0.413 |
| | SAP1- | /i2MOErG/*/i2MOErC/*T*C*C*T*C*G* | | | | | | | |
| | 1850 | G*T*C*T*/i2MOErC/*/i2MOErC/* | | | | | | | |
| | MOE | /i2MOErC/*/i2MOErT/*/32MOErT/ | | | | | | | |
| H8 | NC1 | /52MOErG/*/i2MOErC/*/i2MOErG/* | 2.777 | 1.883 | 0.265 | 0.565 | 0.064 | 0.460 | 0.504 |
| | 2′MOE | /i2MOErT/*/i2MOErA/*T*T*A*T*A*G | | | | | | | |
| | | *C*C*G*A*/i2MOErT/*/i2MOErT/* | | | | | | | |
| | | /i2MOErA/*/i2MOErA/*/32MOErC/ | | | | | | | |
| H9 | MB21 | /52MOErG/*/i2MOErC/*/i2MOErG/* | 2.226 | 1.598 | 0.154 | 0.453 | 0.246 | 0.682 | 0.346 |
| | D1- | /i2MOErG/*/i2MOErA/*G*G*T*C*T*T | | | | | | | |
| | 526 | *G*G*C*T*/i2MOErT/*/i2MOErC/* | | | | | | | |
| | MOE | /i2MOErG/*/i2MOErT/*/32MOErG/ | | | | | | | |

TABLE 5A
Unmodified oliogonucleotide sequences described in Table 5.

| Name | Sequence |
|---|---|
| Unmodified HPRT-489 MOE | ATGTCCCCTGTTGACTGGTC (SEQ ID NO: 522) |
| Unmodified MB21D1-525 MOE | CGGAGGTCTTGGCTTCGTGG (SEQ ID NO: 81) |
| Unmodified HPRT-666 MOE | CCAACACTTCGTGGGGTCCT (SEQ ID NO: 209) |
| Unmodified CTNN2B-311 MOE | GCCGCTTTTCTGTCTGGTTC (SEQ ID NO: 523) |
| Unmodified CTNN2B-1575 MOE | ACCCACTTGGCAGACCATCA (SEQ ID NO: 524) |
| Unmodified EGFR-2790 MOE | CTGTGATCTTGACATGCTGC (SEQ ID NO: 84) |
| Unmodified EGFR-1225 MOE | CAAGGCCCTTCGCACTTCTT (SEQ ID NO: 525) |
| Unmodified CAMSAP1-1635 MOE | GAACCACGTCTGCTCTAACA (SEQ ID NO: 526) |
| Unmodified CAMSAP1-4915 MOE | TTGCACACTTCGTACCCAAA (SEQ ID NO: 527) |
| Unmodified MB21D1-689 MOE | ACAACCCCTTTCACCATCCC (SEQ ID NO: 528) |
| Unmodified HPRT-732 MOE | TAGTCAAGGGCATATCCTAC (SEQ ID NO: 529) |
| Unmodified MB21D1-523 MOE | GAGGTCTTGGCTTCGTGGAG (SEQ ID NO: 530) |
| Unmodified HPRT-664 MOE | AACACTTCGTGGGGTCCTTT (SEQ ID NO: 177) |
| Unmodified CTNN2B-916 MOE | GGGAAAGGTTATGCAAGGTC (SEQ ID NO: 83) |
| Unmodified CTNN2B-808 MOE | CGTGTCTGGAAGCTTCCTTT (SEQ ID NO: 531) |
| Unmodified EGFR-3652 MOE | ACAGTGTTGAGATACTCGGG (SEQ ID NO: 91) |
| Unmodified EGFR-1015 MOE | GTGTCCTTGCACGTGGCTTC (SEQ ID NO: 532) |
| Unmodified CAMSAP1-1739 MOE | CTTACTTTCCGAGGTGTCCA (SEQ ID NO: 533) |
| Unmodified CAMSAP1-5594 MOE | TTGGCTCGGCTTGCCTACTT (SEQ ID NO: 90) |
| Unmodified MB21D1-719 MOE | TCGCACTTCAGTCTGAGCAG (SEQ ID NO: 92) |
| Unmodified HPRT-1027 MOE | AATCCGCCCAAAGGGAACTG (SEQ ID NO: 534) |
| Unmodified MB21D1-522 MOE | AGGTCTTGGCTTCGTGGAGC (SEQ ID NO: 82) |
| Unmodified HPRT-668 MOE | ATCCAACACTTCGTGGGGTC (SEQ ID NO: 172) |
| Unmodified CTNN2B-1294 MOE | CAGATAGCACCTTCAGCACT (SEQ ID NO: 535) |
| Unmodified CTNN2B-2545 MOE | GCCCATCCATGAGGTCCTGG (SEQ ID NO: 173) |
| Unmodified EGFR-3908 MOE | GGGTATCGAAAGAGTCTGGA (SEQ ID NO: 174) |
| Unmodified EGFR-2874 MOE | ATGCCATCCACTTGATAGGC (SEQ ID NO: 536) |
| Unmodified CAMSAP1-4295 MOE | TGGGTTACGGCTCAGTATGG (SEQ ID NO: 183) |
| Unmodified CAMSAP1-4916 MOE | TTTGCACACTTCGTACCCAA (SEQ ID NO: 94) |
| Unmodified MB21D1-1181 MOE | AGTCGTAGTTGCTTCCTAAC (SEQ ID NO: 88) |
| Unmodified HPRT-1353 MOE | GCTGACAAAGATTCACTGGT (SEQ ID NO: 95) |
| Unmodified MB21D1-168 MOE | TCCGGCCTCGGAAGCTCTCT (SEQ ID NO: 138) |
| Unmodified HPRT-663 MOE | ACACTTCGTGGGGTCCTTTT (SEQ ID NO: 170) |
| Unmodified CTNN2B-2341 MOE | GTCCAAGATCAGCAGTCTCA (SEQ ID NO: 178) |
| Unmodified CTNN2B-1642 MOE | TGGCAGGCTCAGTGATGTCT (SEQ ID NO: 537) |
| Unmodified EGFR-4705 MOE | TGGGCTGGAATCCGAGTTAT (SEQ ID NO: 179) |
| Unmodified EGFR-3267 MOE | CGGCATCCACCACGTCGTCC (SEQ ID NO: 180) |
| Unmodified CAMSAP1-4697 MOE | GGTTTTGGCTGGGATCAAGT (SEQ ID NO: 175) |
| Unmodified CAMSAP1-2584 MOE | TTGCCATGCTGGCTCGGACT (SEQ ID NO: 538) |
| Unmodified MB21D1-1248 MOE | CCGCCATGTTTCTTCTTGGA (SEQ ID NO: 184) |
| Unmodified HPRT-665 MOE | CAACACTTCGTGGGGTCCTT (SEQ ID NO: 208) |
| Unmodified MB21D1-520 MOE | GTCTTGGCTTCGTGGAGCAG (SEQ ID NO: 89) |
| Unmodified HPRT-332 MOE | TGTGATGGCCTCCCATCTCC (SEQ ID NO: 304) |
| Unmodified CTNN2B-3648 MOE | CTCTTGAAGCATCGTATCAC (SEQ ID NO: 539) |
| Unmodified CTNN2B-2516 MOE | AGATCTGGCAGCCCATCAAC (SEQ ID NO: 540) |
| Unmodified EGFR-4885 MOE | GGAGATTTCAGAGCAGCTTC (SEQ ID NO: 322) |
| Unmodified EGFR-1013 MOE | GTCCTTGCACGTGGCTTCGT (SEQ ID NO: 321) |
| Unmodified CAMSAP1-6273 MOE | TCAAAGGACTGAGGAAAGGG (SEQ ID NO: 171) |
| Unmodified CAMSAP1-2709 MOE | GCTTCAGGCGCTGCCTTGCC (SEQ ID NO: 541) |
| Unmodified MB21D1-1483 MOE | ACTGACTGTCTTGAGGGTTC (SEQ ID NO: 85) |
| Unmodified HPRT-333 MOE | ATGTGATGGCCTCCCATCTC (SEQ ID NO: 542) |
| Unmodified MB21D1-616 MOE | GGAGCTTCGAGGCCCCAGGC (SEQ ID NO: 14) |
| Unmodified HPRT-335 MOE | CAATGTGATGGCCTCCCATC (SEQ ID NO: 543) |
| Unmodified CTNN2B-809 MOE | GCGTGTCTGGAAGCTTCCTT (SEQ ID NO: 86) |
| Unmodified CTNN2B-2136 MOE | TTCTGCAGCTTCCTTGTCCT (SEQ ID NO: 323) |
| Unmodified EGFR-1014 MOE | TGTCCTTGCACGTGGCTTCG (SEQ ID NO: 302) |
| Unmodified EGFR-1016 MOE | GGTGTCCTTGCACGTGGCTT (SEQ ID NO: 93) |
| Unmodified CAMSAP1-2112 MOE | TGTCTTCATCGGCCCTGCCT (SEQ ID NO: 544) |
| Unmodified CAMSAP1-2456 MOE | GAGTCTCTGGAGCTTCCTCT (SEQ ID NO: 87) |
| Unmodified MB21D1-521 MOE | GGTCTTGGCTTCGTGGAGCA (SEQ ID NO: 139) |
| Unmodified HPRT-667 MOE | TCCAACACTTCGTGGGGTCC (SEQ ID NO: 545) |
| Unmodified MB21D1-697 MOE | GGTGGTCCACAACCCCTTTC (SEQ ID NO: 210) |
| Unmodified CTNN2B-2446 MOE | GGTCCATACCCAAGGCATCC (SEQ ID NO: 305) |
| Unmodified CTNN2B-2479 MOE | GGTGGTGGCCACCCATCTCA (SEQ ID NO: 546) |
| Unmodified EGFR-3266 MOE | GGCATCCACCACGTCGTCCA (SEQ ID NO: 320) |
| Unmodified EGFR-727 MOE | ATGTCCCGCCACTGGATGCT (SEQ ID NO: 547) |
| Unmodified CAMSAP1-2113 MOE | GTGTCTTCATCGGCCCTGCC (SEQ ID NO: 306) |
| Unmodified CAMSAP1-2906 MOE | GTCCACATCCTGTGGCTCGT (SEQ ID NO: 303) |
| Unmodified MB21D1-524 MOE | GGAGGTCTTGGCTTCGTGGA (SEQ ID NO: 182) |
| Unmodified HPRT-329 MOE | GATGGCCTCCCATCTCCTTC (SEQ ID NO: 548) |
| Unmodified NC5 2′MOE | GCGACTATACGCGCAATATG (SEQ ID NO: 176) |
| Unmodified CTNN2B-1576 MOE | CACCCACTTGGCAGACCATC (SEQ ID NO: 549) |
| Unmodified EGFR-2859 MOE | TAGGCACTTTGCCTCCTTCT (SEQ ID NO: 550) |
| Unmodified CAMSAP1-1850 MOE | GATGCTCCTCGGTCTCCCTT (SEQ ID NO: 551) |
| Unmodified NC1 2′MOE | GCGTATTATAGCCGATTAAC (SEQ ID NO: 181) |
| Unmodified MB21D1-526 MOE | GCGGAGGTCTTGGCTTCGTG (SEQ ID NO: 96) |

TABLE 6
3mer 2′OMe ASOs

| | SEQ | | | | | | | TLR8 | | |
|---|---|---|---|---|---|---|---|---|---|---|
| | ID | | | | TLR7 | TLR7 | TLR8 | HEK | TLR9 | TLR3 |
| 3mer | NO | Ref | Seq | Well | 400 nM | 2 uM | 1 uM | 5 uM | 2 uM | 2 uM |
| AAA | 365 | 1 | mA* | A1 | 1.352 | 0.867 | 225.029 | 0.859 | 0.845 | 1.026 |
| | | | mA*mA | | | | | | | |
| AGC | 374 | 10 | mA* | A10 | 1.401 | 0.726 | 267.225 | 1.411 | 0.749 | 1.019 |
| | | | mG*mC | | | | | | | |
| AAC | 373 | 2 | mA* | A2 | 1.019 | 0.770 | 218.889 | 0.696 | 0.931 | 0.901 |
| | | | mA*mC | | | | | | | |
| AAG | 380 | 3 | mA* | A3 | 1.247 | 0.876 | 179.302 | 0.858 | 0.870 | 0.616 |
| | | | mA*mG | | | | | | | |
| AAU | 426 | 4 | mA* | A4 | 1.340 | 0.888 | 202.802 | 0.807 | 0.884 | 0.888 |
| | | | mA*mU | | | | | | | |
| ACA | 168 | 5 | mA* | A5 | 1.234 | 0.831 | 262.630 | 0.841 | 1.042 | 1.053 |
| | | | mC*mA | | | | | | | |
| ACC | 403 | 6 | mA* | A6 | 1.290 | 0.795 | 262.122 | 0.939 | 0.604 | 0.850 |
| | | | mC*mC | | | | | | | |
| ACG | 411 | 7 | mA* | A7 | 1.212 | 0.825 | 185.962 | 1.328 | 0.512 | 0.993 |
| | | | mC*mG | | | | | | | |
| ACU | 427 | 8 | mA* | A8 | 1.322 | 0.916 | 250.907 | 0.896 | 0.996 | 1.062 |
| | | | mC*mU | | | | | | | |
| AGA | 366 | 9 | mA* | A9 | 1.219 | 0.795 | 214.450 | 1.520 | 0.848 | 0.829 |
| | | | mG*mA | | | | | | | |
| AGG | 381 | 11 | mA* | B1 | 1.335 | 0.866 | 193.033 | 2.596 | 1.005 | 0.855 |
| | | | mG*mG | | | | | | | |
| CAU | 428 | 20 | mC* | B10 | 1.288 | 0.995 | 388.191 | 0.752 | 0.921 | 0.866 |
| | | | mA*mU | | | | | | | |
| AGU | 429 | 12 | mA* | B2 | 1.025 | 0.952 | 270.256 | 1.329 | 0.840 | 0.829 |
| | | | mG*mU | | | | | | | |
| AUA | 430 | 13 | mA* | B3 | 1.171 | 0.905 | 157.293 | 0.812 | 0.944 | 0.570 |
| | | | mU*mA | | | | | | | |
| AUC | 431 | 14 | mA* | B4 | 0.950 | 0.430 | 181.793 | 0.674 | 0.884 | 0.768 |
| | | | mU*mC | | | | | | | |
| AUG | 432 | 15 | mA* | B4 | 0.987 | 0.602 | 151.847 | 0.828 | 0.878 | 0.859 |
| | | | mU*mG | | | | | | | |
| AUU | 433 | 16 | mA* | B6 | 1.221 | 0.700 | 274.683 | 0.762 | 0.864 | 0.809 |
| | | | mU*mU | | | | | | | |
| CAA | 367 | 17 | mC* | B7 | 1.175 | 0.787 | 373.157 | 0.811 | 0.917 | 0.771 |
| | | | mA*mA | | | | | | | |
| CAC | 169 | 18 | mC* | B8 | 1.330 | 0.932 | 344.415 | 0.809 | 0.830 | 0.841 |
| | | | mA*mC | | | | | | | |
| CAG | 382 | 19 | mC* | B9 | 1.271 | 1.057 | 362.782 | 0.960 | 0.898 | 0.743 |
| | | | mA*mG | | | | | | | |
| CCA | 397 | 21 | mC* | C1 | 1.270 | 0.780 | 181.239 | 0.946 | 1.014 | 0.917 |
| | | | mC*mA | | | | | | | |
| CUC | 434 | 30 | mC* | C10 | 1.288 | 0.984 | 245.566 | 0.685 | 0.966 | 0.874 |
| | | | mU*mC | | | | | | | |
| CCC | 405 | 22 | mC* | C2 | 1.196 | 0.993 | 181.399 | 0.756 | 1.069 | 0.933 |
| | | | mC*mC | | | | | | | |
| CCG | 413 | 23 | mC* | C3 | 1.227 | 1.006 | 437.264 | 0.899 | 0.936 | 0.770 |
| | | | mC*mG | | | | | | | |
| CCU | 435 | 24 | mC* | C4 | 1.210 | 0.880 | 332.524 | 0.613 | 1.180 | 0.870 |
| | | | mC*mU | | | | | | | |
| CGA | 368 | 25 | mC* | C5 | 1.112 | 0.907 | 504.099 | 1.512 | 1.061 | 0.780 |
| | | | mG*mA | | | | | | | |
| CGC | 375 | 26 | mC* | C6 | 1.282 | 0.947 | 806.706 | 1.834 | 0.648 | 0.749 |
| | | | mG*mC | | | | | | | |
| CGG | 383 | 27 | mC* | C7 | 1.412 | 0.791 | 2548.596 | 2.877 | 1.078 | 0.847 |
| | | | mG*mG | | | | | | | |
| CGU | 436 | 28 | mC* | C8 | 1.372 | 0.963 | 346.350 | 1.327 | 1.237 | 0.833 |
| | | | mG*mU | | | | | | | |
| CUA | 437 | 29 | mC* | C9 | 1.388 | 0.953 | 243.564 | 0.642 | 1.182 | 0.851 |
| | | | mU*mA | | | | | | | |
| CUG | 438 | 31 | mC* | D1 | 1.405 | 0.804 | 264.072 | 1.074 | 0.948 | 0.870 |
| | | | mU*mG | | | | | | | |
| GCU | 439 | 40 | mG* | D10 | 1.339 | 0.852 | 193.279 | 0.810 | 1.429 | 0.808 |
| | | | mC*mU | | | | | | | |
| CUU | 153 | 32 | mC* | D2 | 1.277 | 0.913 | 167.148 | 0.712 | 1.108 | 0.846 |
| | | | mU*mU | | | | | | | |
| GAA | 369 | 33 | mG* | D3 | 1.233 | 0.433 | 138.047 | 0.647 | 0.863 | 0.786 |
| | | | mA*mA | | | | | | | |
| GAC | 376 | 34 | mG* | D4 | 1.138 | 0.505 | 141.925 | 0.494 | 1.213 | 0.832 |
| | | | mA*mC | | | | | | | |
| GAG | 384 | 35 | mG* | D5 | 0.958 | 0.465 | 130.205 | 0.471 | 1.047 | 0.844 |
| | | | mA*mG | | | | | | | |
| GAU | 440 | 36 | mG* | D6 | 1.406 | 0.580 | 147.162 | 0.528 | 0.700 | 0.771 |
| | | | mA*mU | | | | | | | |
| GCA | 399 | 37 | mG* | D7 | 1.240 | 0.717 | 218.474 | 0.786 | 1.047 | 0.798 |
| | | | mC*mA | | | | | | | |
| GCC | 407 | 38 | mG* | D8 | 1.360 | 0.645 | 209.536 | 0.701 | 1.305 | 0.884 |
| | | | mC*mC | | | | | | | |
| GCG | 415 | 39 | mG* | D9 | 1.185 | 0.627 | 290.409 | 1.191 | 1.299 | 0.830 |
| | | | mC*mG | | | | | | | |
| GGA | 370 | 41 | mG* | E1 | 1.053 | 0.474 | 195.033 | 1.919 | 0.853 | 0.979 |
| | | | mG*mA | | | | | | | |
| UAC | 441 | 50 | mU* | E10 | 1.161 | 1.003 | 254.486 | 0.813 | 0.997 | 0.813 |
| | | | mA*mC | | | | | | | |
| GGC | 377 | 42 | mG* | E2 | 0.659 | 0.259 | 151.618 | 1.697 | 1.009 | 0.792 |
| | | | mG*mC | | | | | | | |
| GGG | 385 | 43 | mG* | E3 | 1.141 | 0.680 | 214.414 | 1.147 | 0.734 | 0.849 |
| | | | mG*mG | | | | | | | |
| GGU | 59 | 44 | mG* | E4 | 0.938 | 0.847 | 274.989 | 1.380 | 1.177 | 0.829 |
| | | | mG* | | | | | | | |
| | | | mU | | | | | | | |
| GUA | 60 | 45 | mG* | E5 | 0.702 | 0.239 | 133.537 | 0.792 | 1.042 | 0.839 |
| | | | mU* | | | | | | | |
| | | | mA | | | | | | | |
| GUC | 442 | 46 | mG* | E6 | 0.140 | 0.017 | 131.948 | 0.763 | 0.856 | 0.752 |
| | | | mU*mC | | | | | | | |
| GUG | 443 | 47 | mG* | E7 | 0.449 | 0.072 | 158.982 | 0.882 | 1.374 | 0.786 |
| | | | mU*mG | | | | | | | |
| GUU | 444 | 48 | mG* | E8 | 0.781 | 0.119 | 166.278 | 0.668 | 1.159 | 0.825 |
| | | | mU*mU | | | | | | | |
| UAA | 445 | 49 | mU* | E9 | 1.126 | 1.029 | 224.188 | 0.907 | 1.176 | 0.790 |
| | | | mA*mA | | | | | | | |
| UAG | 446 | 51 | mU* | F1 | 1.158 | 0.918 | 226.441 | 1.443 | 0.977 | 0.940 |
| | | | mA*mG | | | | | | | |
| UGU | 447 | 60 | mU* | F10 | 1.108 | 0.984 | 331.923 | 1.120 | 1.085 | 0.967 |
| | | | mG*mU | | | | | | | |
| UAU | 448 | 52 | mU* | F2 | 1.086 | 0.935 | 213.758 | 0.887 | 1.145 | 0.915 |
| | | | mA*mU | | | | | | | |
| UCA | 449 | 53 | mU* | F3 | 1.122 | 0.819 | 1049.345 | 1.115 | 0.872 | 0.765 |
| | | | mC*mA | | | | | | | |
| UCC | 450 | 54 | mU* | F4 | 1.166 | 0.958 | 346.575 | 1.292 | 1.106 | 0.761 |
| | | | mC*mC | | | | | | | |
| UCG | 451 | 55 | mU* | F5 | 0.979 | 0.982 | 746.272 | 2.839 | 0.514 | 0.850 |
| | | | mC*mG | | | | | | | |
| UCU | 452 | 56 | mU* | F6 | 1.196 | 0.956 | 371.961 | 1.414 | 0.876 | 0.804 |
| | | | mC*mU | | | | | | | |
| UGA | 453 | 57 | mU* | F7 | 1.126 | 0.831 | 267.234 | 1.331 | 1.034 | 0.806 |
| | | | mG*mA | | | | | | | |
| UGC | 454 | 58 | mU* | F8 | 1.057 | 0.782 | 278.054 | 1.032 | 1.198 | 0.903 |
| | | | mG*mC | | | | | | | |
| UGG | 455 | 59 | mU* | F9 | 1.029 | 0.989 | 527.575 | 1.860 | 1.137 | 0.764 |
| | | | mG*mG | | | | | | | |
| UUA | 456 | 61 | mU* | G1 | 0.997 | 1.042 | 350.207 | 1.060 | 0.912 | 1.081 |
| | | | mU*mA | | | | | | | |
| NT | | | NT | G10 | 0.000 | 0.000 | | 0.020 | 0.005 | 0.000 |
| UUC | 457 | 62 | mU* | G2 | 0.536 | 0.642 | 213.878 | 0.968 | 0.770 | 1.023 |
| | | | mU*mC | | | | | | | |
| UUG | 458 | 63 | mUm | G3 | 0.945 | 1.074 | 347.573 | 1.071 | 0.816 | 1.030 |
| | | | U*mG | | | | | | | |
| UUU | 459 | 64 | mU* | G4 | 0.861 | 1.084 | 307.213 | 0.870 | 0.936 | 0.970 |
| | | | mU*mU | | | | | | | |
| Ligand | | | Ligand | | 1.000 | 1.000 | 355.752 | 1.000 | 1.000 | 1.000 |
| NT | | | NT | | 0.000 | 0.000 | 134.831 | 0.000 | 0.000 | 0.000 |

TABLE 7
3 mer DNA ASOs

| | SEQ | | | | TLR3 |
|---|---|---|---|---|---|
| 3 mer | ID NO | Ref | Seq | Position | 2uM |
| AAA | 365 | 1 | dA*dA*dA | A1 | 1.065 |
| AGA | 366 | 9 | dA*dG*dA | A2 | 0.849 |
| CAA | 367 | 17 | dC*dA*dA | A3 | 0.909 |
| CGA | 368 | 25 | dC*dG*dA | A4 | 0.823 |
| GAA | 369 | 33 | dG*dA*dA | A5 | 1.059 |
| GGA | 370 | 41 | dG*dG*dA | A6 | 1.033 |
| TAA | 371 | 49 | dT*dA*dA | A7 | 0.968 |
| TGA | 372 | 57 | dT*dG*dA | A8 | 0.743 |
| AAC | 373 | 2 | dA*dA*dC | B1 | 0.861 |
| AGC | 374 | 10 | dA*dG*dC | B2 | 0.854 |
| CAC | 169 | 18 | dC*dA*dC | B3 | 0.796 |
| CGC | 375 | 26 | dC*dG*dC | B4 | 0.775 |
| GAC | 376 | 34 | dG*dA*dC | B5 | 0.757 |
| GGC | 377 | 42 | dG*dG*dC | B6 | 0.936 |
| TAC | 378 | 50 | dT*dA*dC | B7 | 0.704 |
| TGC | 379 | 58 | dT*dG*dC | B8 | 0.795 |
| AAG | 380 | 3 | dA*dA*dG | C1 | 0.825 |
| AGG | 381 | 11 | dA*dG*dG | C2 | 0.848 |
| CAG | 382 | 19 | dC*dA*dG | C3 | 0.767 |
| CGG | 383 | 27 | dC*dG*dG | C4 | 0.804 |
| GAG | 384 | 35 | dG*dA*dG | C5 | 0.856 |
| GGG | 385 | 43 | dG*dG*dG | C6 | 0.846 |
| TAG | 386 | 51 | dT*dA*dG | C7 | 0.769 |
| TGG | 387 | 59 | dT*dG*dG | C8 | 0.773 |
| AAT | 388 | 4 | dA*dA*dT | D1 | 0.877 |
| AGT | 389 | 12 | dA*dG*dT | D2 | 0.865 |
| CAT | 390 | 20 | dC*dA*dT | D3 | 0.828 |
| CGT | 391 | 28 | dC*dG*dT | D4 | 0.755 |
| GAT | 392 | 36 | dG*dA*dT | D5 | 0.864 |
| GGT | 393 | 44 | dG*dG*dT | D6 | 0.959 |
| TAT | 394 | 52 | dT*dA*dT | D7 | 0.877 |
| TGT | 395 | 60 | dT*dG*dT | D8 | 0.884 |
| ACA | 168 | 5 | dA*dC*dA | E1 | 0.832 |
| ATA | 396 | 13 | dA*dT*dA | E2 | 0.827 |
| CCA | 397 | 21 | dC*dC*dA | E3 | 0.763 |
| CTA | 398 | 29 | dC*dT*dA | E4 | 0.864 |
| GCA | 399 | 37 | dG*dC*dA | E5 | 0.730 |
| GTA | 400 | 45 | dG*dT*dA | E6 | 0.818 |
| TCA | 401 | 53 | dT*dC*dA | E7 | 0.792 |
| TTA | 402 | 61 | dT*dT*dA | E8 | 0.855 |
| ACC | 403 | 6 | dA*dC*dC | F1 | 1.015 |
| ATC | 404 | 14 | dA*dT*dC | F2 | 0.834 |
| CCC | 405 | 22 | dC*dC*dC | F3 | 0.811 |
| CTC | 406 | 30 | dC*dT*dC | F4 | 0.911 |
| GCC | 407 | 38 | dG*dC*dC | F5 | 0.895 |
| GTC | 408 | 46 | dG*dT*dC | F6 | 0.914 |
| TCC | 409 | 54 | dT*dC*dC | F7 | 0.868 |
| TTC | 410 | 62 | dT*dT*dC | F8 | 0.928 |
| ACG | 411 | 7 | dA*dC*dG | G1 | 0.831 |
| ATG | 412 | 15 | dA*dT*dG | G2 | 0.849 |
| CCG | 413 | 23 | dC*dC*dG | G3 | 0.867 |
| CTG | 414 | 31 | dC*dT*dG | G4 | 0.843 |
| GCG | 415 | 39 | dG*dC*dG | G5 | 0.867 |
| GTG | 416 | 47 | dG*dT*dG | G6 | 0.929 |
| TCG | 417 | 55 | dT*dC*dG | G7 | 0.931 |
| TTG | 418 | 63 | dT*dT*dG | G8 | 0.990 |
| ACT | 419 | 8 | dA*dC*dT | H1 | 0.818 |
| ATT | 420 | 16 | dA*dT*dT | H2 | 1.012 |
| CCT | 421 | 24 | dC*dC*dT | H3 | 0.914 |
| CTT | 203 | 32 | dC*dT*dT | H4 | 0.901 |
| GCT | 422 | 40 | dG*dC*dT | H5 | 0.998 |
| GTT | 423 | 48 | dG*dT*dT | H6 | 1.009 |
| TCT | 424 | 56 | dT*dC*dT | H7 | 1.230 |
| TTT | 425 | 64 | dT*dT*dT | H8 | 1.139 |
| Ligand | | | | | 1.000 |
| NT | | | | | 0.000 |

### EXAMPLES

### Example 1: Methods

### Cell Isolation, Culture and Stimulation

Human Primary bone marrow-derived mesenchymal stem cell (MSCs) from 2 healthy adult donors (#1129 and #1980) were purchased from Lonza (#PT-2501) and were cultured in Dulbecco's modified Eagle's medium plus L-glutamine supplemented with 1× antibiotic/antimycotic (Thermo Fisher Scientific) and 10% heat-inactivated foetal bovine serum (referred to as complete DMEM). Culture media was replaced twice a week and cells were passaged at 80% confluency and seeded at a density of 2.5×10′ cells/cm². These cells were confirmed free from pathogens and certified to meet MSC criteria as defined by the International Society of Cell and Gene Therapy. MSCs used herein were plated at passage 6-7. Rheumatoid arthritis (RA) patients fulfilled the American College of Rheumatology (ACR) criteria for the classification of RA (Arnett et al., 1987). RA and control primary fibroblast-like synoviocytes (FLS) were obtained from surgical specimens of synovial tissue and cultured as previously described (Leech et al., 1999). FLS were grown in RPMI 1640 plus L-glutamine medium (Life Technologies) complemented with 1× antibiotic/antimycotic and 10% heat inactivated foetal bovine serum (referred to as complete RPMI).

Trex1-mutant mice (used under animal ethics ref A2018/38) have a single-based mutation in Trex1 leading to a premature stop codon (Q169X) and aberrant accumulation of cytoplasmic DNA, resulting in basal engagement of the cGAS-STING pathway (Ellyard J. I. and Vinuesa C. G., manuscript in preparation), similar to that reported in Trex1-deficient mice (Gray et al., 2015). Primary bone marrow derived macrophages (BMDMs) from wild-type, Trex1-mutant or Tlr7^{Y264}H mutant mice were extracted and differentiated for 6 days in complete DMEM supplemented with L929 conditioned medium as previously reported (Ferrand and Gantier, 2016). 293XL-hTLR7-HA, 293XL-hTLR9-HA and HEK-Blue™ hTLR3 stably expressing human TLR7, TLR9 or TLR3 were purchased from Invivogen, and were maintained in complete DMEM supplemented with 10 μg/ml and 30 μg/ml Blasticidin (Invivogen), for TLR7/9 and TLR3 cells, respectively. HeLa cells, human colorectal cancer HT-29 (kind gift from R. Firestein), human fibroblasts expressing SV40 (large and small T antigens) with HRASG12V (kind gift from E. Sanij (Quin et al., 2016); referred to as BJ hTERT SV40T herein), LL171 cells (mouse L929 cells expressing an IFN stimulated response element (ISRE)-Luciferase—kind gift from V. Homung (Ablasser et al., 2013), and immortalized wild-type mouse bone marrow macrophages (BMDMs) (Ferrand et al., 2018) were grown in complete DMEM. Human osteosarcoma MG-63 cells were purchased from ATCC (#CRL-1427) and grown in ATCC-formulated Eagle's Minimum Essential Medium, supplemented with 10% heat-inactivated foetal bovine serum (Thermo Fisher Scientific) and 1× antibiotic/antimycotic (Thermo Fisher Scientific). Human acute myeloid leukemia THP-1 and their CRISPR-Cas9 derivatives (cGAS−/− (Mankan et al., 2014), UNC93B1−/− and UNC93B1−/− reconstituted with UNC93B1 (Pelka et al., 2014)) cells were grown in complete RPMI. THP-1 cells were not differentiated with PMA in any experiments, and rather used in suspension. All the cells were cultured at 37° C. with 5% CO₂. Cell lines were passaged 2-3 times a week and tested for mycoplasma contamination on routine basis by PCR.

For cGAS stimulations, cells were treated for indicated duration with ASOs, prior to transfection with ISD70 (human cells) or ISD45 (mouse cells) (the ASOs were not washed off prior to ISD transfection unless otherwise indicated). The cGAS ligands ISD45 (Stetson and Medzhitov, 2006) and ISD70 (also known as VACV-70 (Unterholzner et al., 2010)) (Table 1) were resuspended as follows: 5 1d of sense and 5 μl of antisense strands at 10 μg/μl were added to 90 μl PBS under sterile conditions, heated at 75° C. for 30 min, prior to letting cool down at room temperature and aliquoting (stock at 1 μg/1 l). The ISDs were transfected at a concentration of 2.5 pg/ml at a ratio of 1 μg:1 μl with Lipofectamine 2000 in Opti-MEM (Thermo Fisher Scientific). HEK-TLR3, HEK-TLR7 and HEK-TLR9 were treated with indicated concentration of ASOs for 20-50 min, prior to stimulation with poly(I:C) (Invivogen), R848 (Invivogen), Motolimod (MedChemExpress) and the Class B CpG oligonucleotides ODN 2006 (synthesised by IDT and resuspended in RNase-free TE buffer [Table 1]), respectively. The TLR2/1 agonist PAM3CSK4 (Invivogen), the TLR4 agonist lipopolysaccharide (Invivogen), the Class B CpG oligonucleotides ODN 1826 (synthesised by IDT and resuspended in RNase-free TE buffer), the mouse Sting agonist DMXAA (Cayman) and the human STING agonist (compound #3 from (Raanjulu et al., 2018), referred to as GSK herein—kind gift from Cancer Therapeutics CRC, Australia) were used at indicated concentrations. Aspirin (Sigma—A2093) was resuspended in pure medium to 10 mM, filter sterilised and added to the cells at 2 mM final. All ASOs were synthesised by Integrated DNA Technologies (IDT), and resuspended in RNase-free TE buffer, pH 8.0 (Thermo Fisher Scientific). ASO sequences and modifications are provided in Tables 1 and 2. Cell viability was assessed by adding 1× fresh resazurin solution to the wells (10λ solution made up with 7 mg resazurin [Sigma—R7017] dissolved in 3.5 ml PBS, sterile filtered at 0.2 μM) for 4 h, prior to reading at with a Fluostar OPTIMA (BMG LABTECH) plate-reader (fluorescence: Excitation 535 nm, Emission 590); wells with medium only and 1× resazurin were used as blanks.

### Luciferase Assays

HEK293 cells stably expressing TLR3, 7 or 9 were reverse-transfected with pNF-κB-Luc4 reporter (Clontech), with Lipofectamine 2000 (Thermo Fisher Scientific), according to the manufacturer's protocol. Briefly, 500,000-700,000 cells were reverse-transfected with 400 ng of reporter with 1.2 μl of Lipofectamine 2000 per well of a 6-well plate, and incubated for 3-24 h at 37° C. with 5% CO₂. Following transfection, the cells were collected from the 6-wells and aliquoted into 96-wells, just before ASO and overnight TLR stimulation (as above described). Similarly, the LL171 cells expressing and ISRE-Luc reporter were treated overnight. The next day, the cells were lysed in 40 μl (for a 96-well plate) of 1× Glo Lysis buffer (Promega) for 10 min at room temperature. μl of the lysate was then subjected to firefly luciferase assay using 40 μl of Luciferase Assay Reagent (Promega). Luminescence was quantified with a Fluostar OPTIMA (BMG LABTECH) luminometer.

### Beta-Galactosidase Staining

A β-Galactosidase staining assay was performed on FLS and MSCs treated with ASOs using the Senescence β-Galactosidase Staining kit (New England Biolabs). Briefly, FLS and MSCs were washed with PBS, fixed and stained over 24-48 h with X-Gal solution at 37° C. according to the manufacturer's protocol. The cells were imaged using inverted phase microscopy. Three to 6 images were taken per condition and analysed with image J, counting the number of β-Galactosidase positive cells (blue) per image (totaling>100 cells for each condition). The relative proportion of blue cells per field was calculated for each image.

### ASO Reverse-Transfections

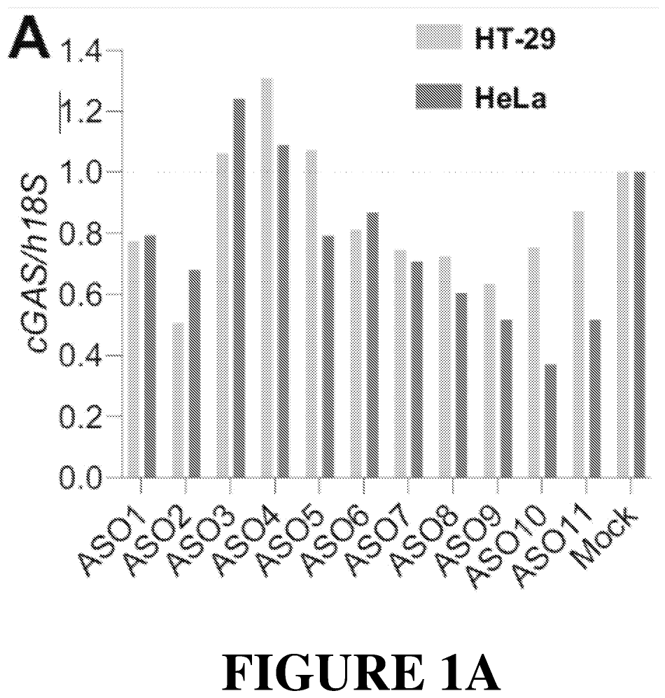

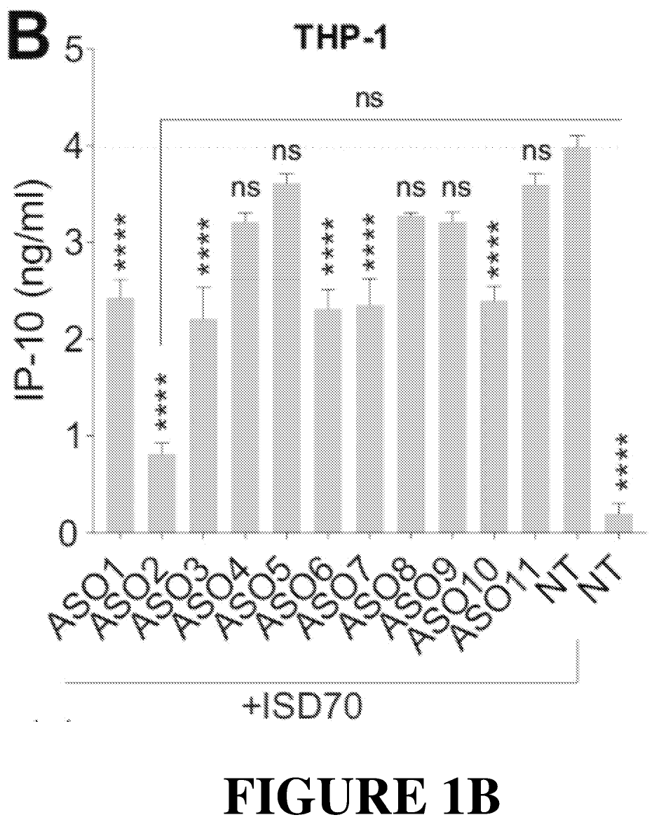

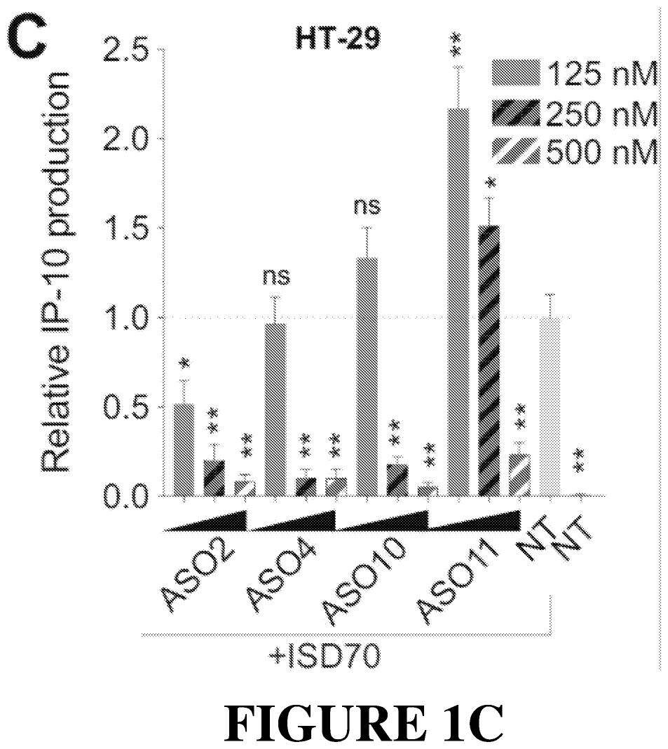

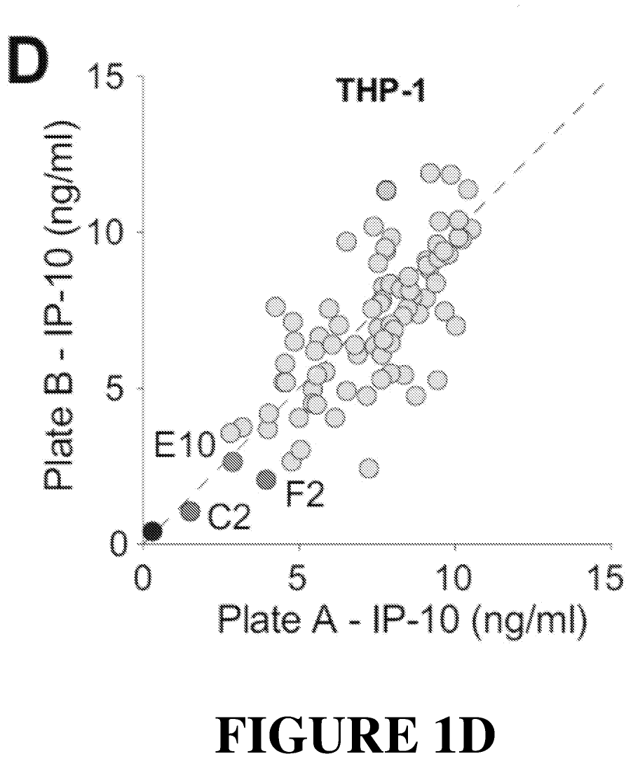

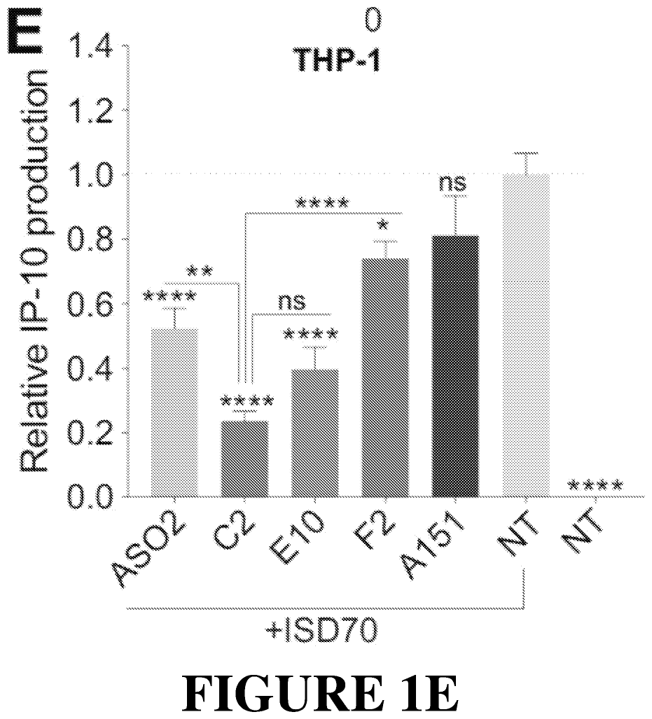

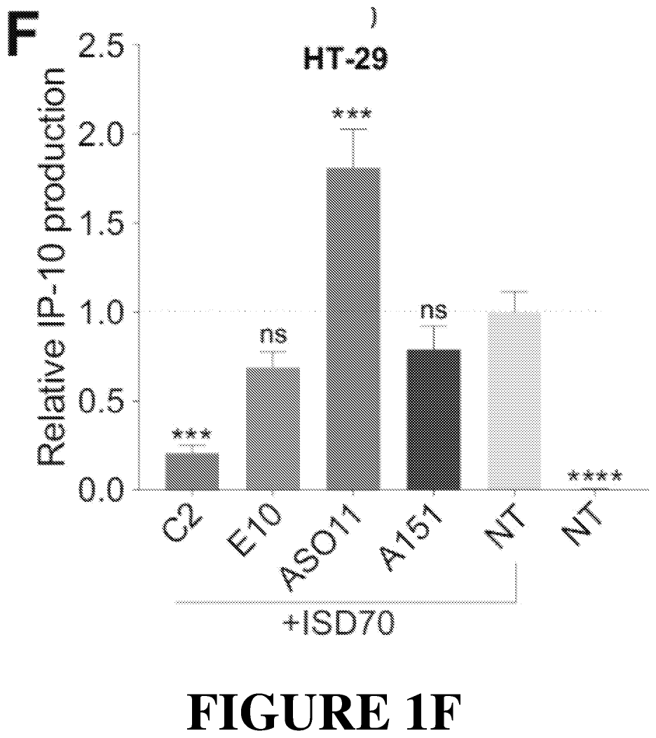

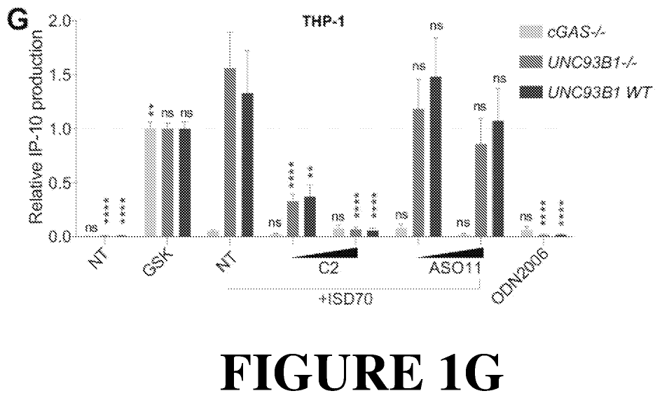

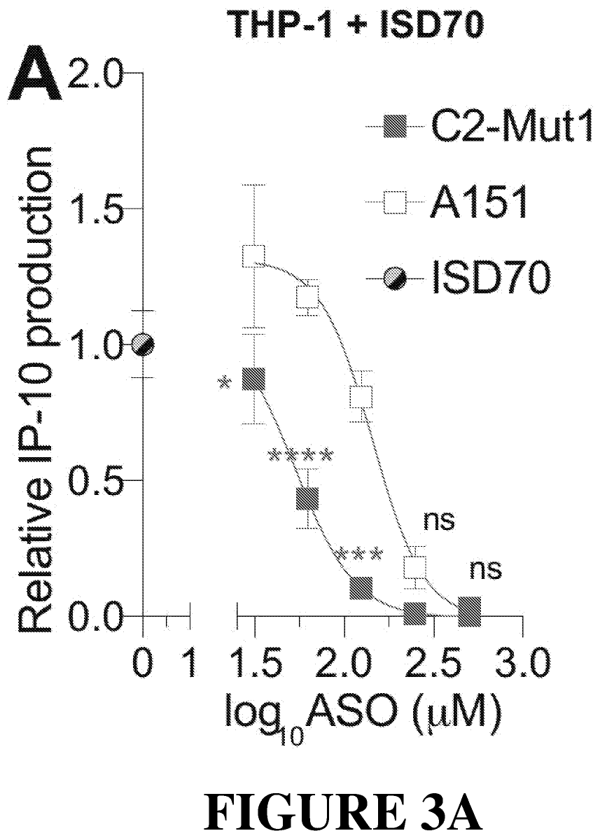

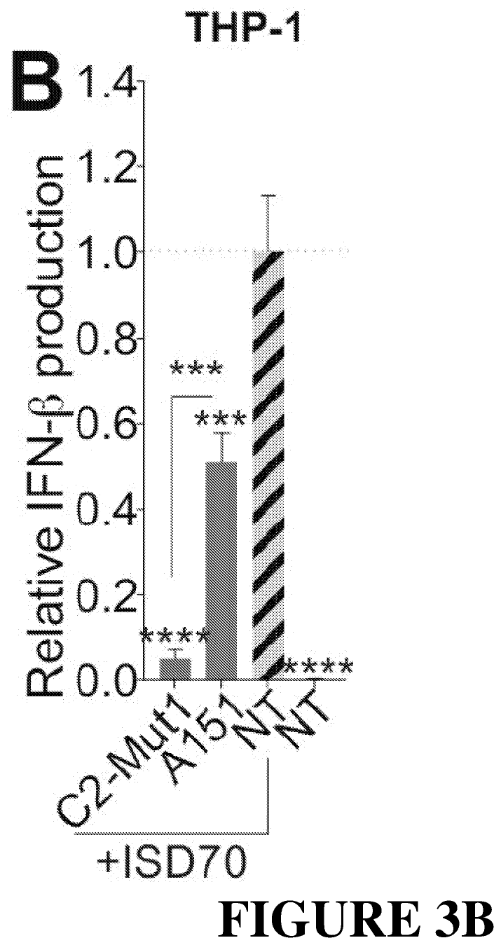

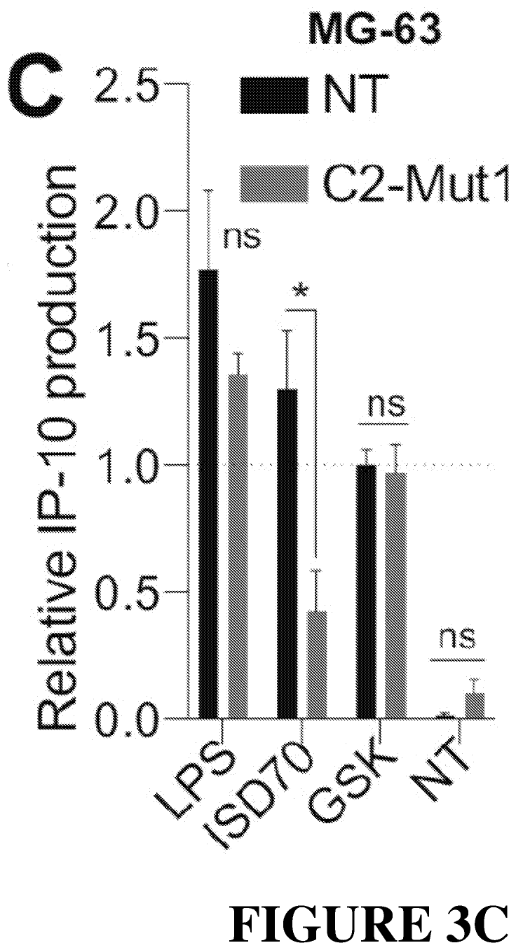

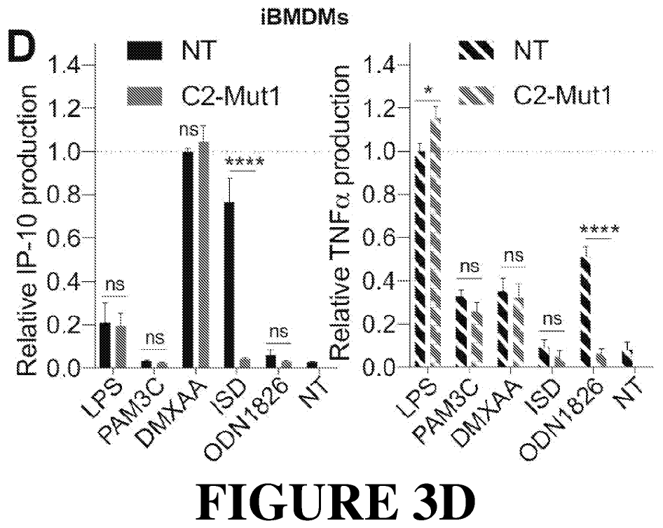

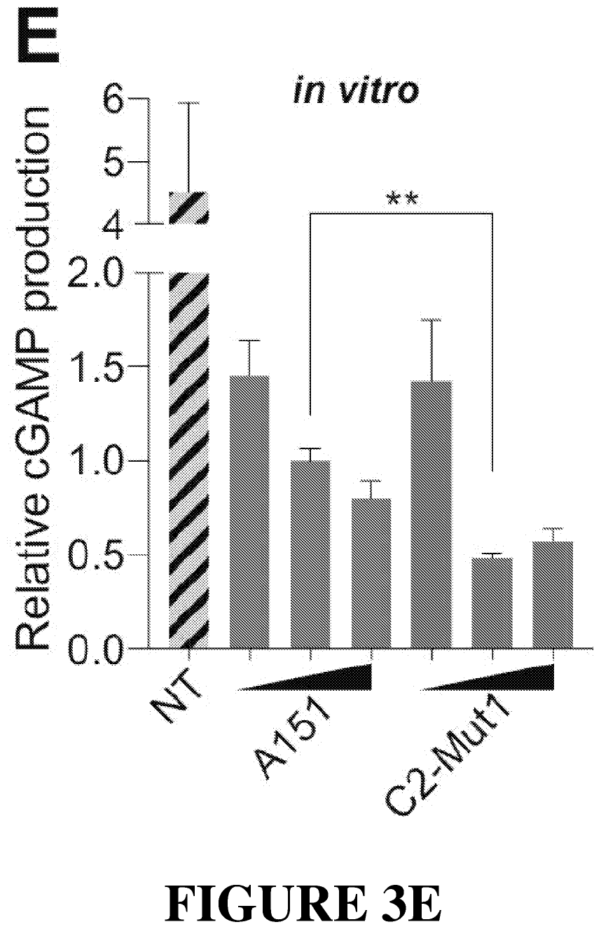

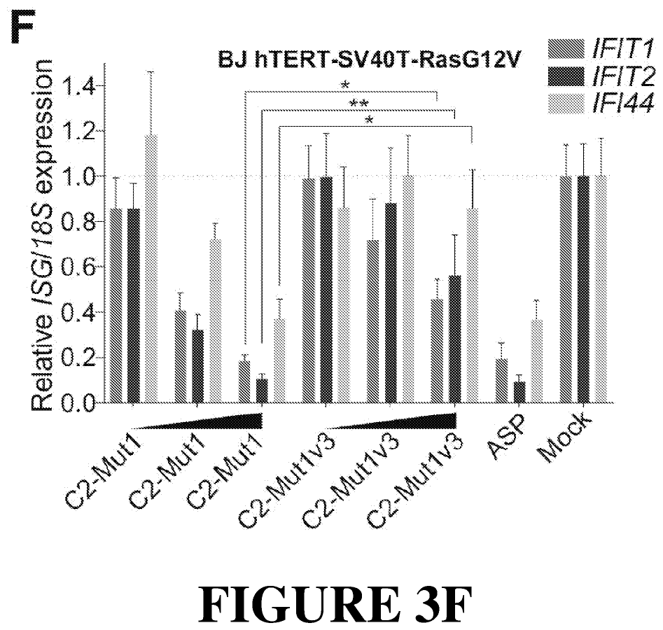

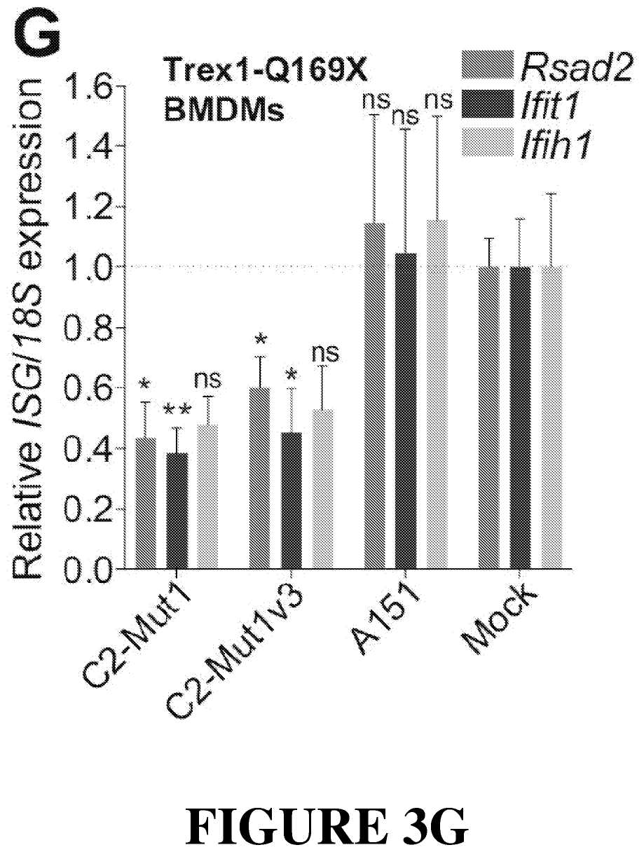

For FIGS. 1A, 3F and 3G, ASOs were reverse-transfected with Lipofectamine 2000. Briefly, a solution of 1.125 μl Lipofectamine 2000 in 25 μl Opti-MEM was mixed to a solution of 1 μl of 10 M ASO in 25 μL Opti-MEM. Following 20-25 min incubation at room temperature, the resulting 50 1d solution was dispensed into a 24-well, and 50-80,000 cells in 450 μl antibiotics-free medium were added, giving a final ASO concentration of 20 nM per well. For conditions with 50 and 100 nM ASOs (FIG. 3F), the amount of Lipofectamine was kept constant. Cells were lysed in 150 μl RNA lysis Buffer (ISOLATE II RNA Mini Kit) after 24 h incubation.

### RNA Reverse Transcription Quantitative Real-Time PCR (RT-qPCR)

Total RNA was purified from cells using the ISOLATE II RNA Mini Kit (Bioline). Random hexamer cDNA was synthesized from isolated RNA using the High-Capacity cDNA Archive kit (Thermo Fisher Scientific) according to the manufacturer's instructions. RT-qPCR was carried out with the Power SYBR Green Master Mix (Thermo Fisher Scientific) on the HT7900 and QuantStudio 6 RT-PCR system (Thermo Fisher Scientific). Each PCR was carried out in technical duplicate and human or mouse 18S was used as reference gene. Each amplicon was gel-purified and used to generate a standard curve for the quantification of gene expression (used in each run). Melting curves were used in each run to confirm specificity of amplification. The primers used were the following: Human hIFIT1: hIFIT1-FWD TCACCAGATAGGGCTTTGCT (SEQ ID NO: 324); hIFIT1-REV CACCTCAAATGTGGGCTTTT (SEQ ID NO: 325); h18S: h18S-FWD CGGCTACCACATCCAAGGAA (SEQ ID NO: 326); h18S-REV GCTGGAATTACCGCGGCT (SEQ ID NO: 327); hIFI44: hIFI44-FWD ATGGCAGTGACAACTCGTTTG (SEQ ID NO: 328); hIFI44: TCCTGGTAACTCTCTTCTGCATA (SEQ ID NO: 329); Human IFIT2: hIFIT2-RT-FWD TTATTGGTGGCAGAAGAGGAAG (SEQ ID NO: 330); hIFIT2-RT-REV CCTCCATCAAGTTCCAGGTG (SEQ ID NO: 331); Human cGAS: hcGAS-FWD CACGTATGTACCCAGAACCC (SEQ ID NO: 332); hcGAS-REV GTCCTGAGGCACTGAAGAAAG (SEQ ID NO: 333); Mouse Rsad2: mRsad2-FWD CTGTGCGCTGGAAGGTTT (SEQ ID NO: 334); mRsad2-REV ATTCAGGCACCAAACAGGAC (SEQ ID NO: 335); Mouse 18S: mRnl8s-FWD GTAACCCGTTGAACCCCATT (SEQ ID NO: 336); mRnl8s-REV CCATCCAATCGGTAGTAGCG (SEQ ID NO: 337); Mouse Ifihl: mlfihl-FWD TCTTGGACACTTGCTTCGAG (SEQ ID NO: 338); mlfihl-REV TCCTTCTGCACAATCCTTCTC (SEQ ID NO: 339); Mouse Ifitl: mIfitl-RT-FWD GAGAGTCAAGGCAGGTTTCT (SEQ ID NO: 340); mIfitl-RT-REV TCTCACTTCCAAATCAGGTATGT (SEQ ID NO: 341). Mouse OAS3: mOAS3-FWD GTACCACCAGGTGCAGACAC (SEQ ID NO: 342); mOAS3-REV GCCATAGTTTTCCGTCCAGA (SEQ ID NO: 343);

### Detection of Cytokines

Human IP-10 and IFN-β levels were measured using supernatants from the different cultures and were quantified using IP-10 (BD Biosciences, #550926) or IFN-β (PBL assay science, #41415-1) ELISA kits respectively, according to the manufacturers' protocol. Tetramethylbenzidine substrate (Thermo Fisher Scientific) was used for quantification of the cytokines on a Fluostar OPTIMA (BMG LABTECH) plate-reader.

cGAS in vitro Assay and cGAMP ELISA

0.8 μg of Recombinant full-length human cGAS (Cayman, #22810) was used per single reaction in a 200 μl volume with 80 mM Tris-HCl (pH 7.5), 200 mM NaCl, 20 μM ZnCl₂, 20 mM MgCl₂, 0.25 mM GTP (Thermofisher #R0441) and 0.25 mM ATP (Thermofisher #R0461), with 20 μg of ISD70 (freshly annealed at 1 μg/l in PBS), and 2 μl C2-Mut1/A151 diluted in TE buffer (to get 0.5, 2 or 10 μM ODN concentration in 200 l). After 40 min at 37° C., 2.5 mM EDTA was added to each tube to stop the enzymatic reaction and the samples were either snap frozen and stored at −80° C. or directly processed for cGAMP ELISA. cGAMP levels were measured with the DetectX Direct 2′,3′-Cyclic GAMP Enzyme Immunoassay Kit (Arbor Assays), according to the manufacturer's protocol. Briefly, 50 μL of in vitro reaction were added per well of the kit microplate with 50 μL Assay buffer, 25 μL conjugate, and 25 μL Antibody per well, prior to a minimum of 2 h incubation. The standards were prepared with serial-dilution in Assay buffer. Quantification of cGAMP levels was performed on a Fluostar OPTIMA (BMG LABTECH) plate-reader at 450 nm.

### Statistical Analyses

Statistical analyses were carried out using Prism 8 (GraphPad Software Inc.). Every experiment was repeated a minimum of two independent times (except the ASO screens—FIGS. 1D and 4D, for which key ASOs were validated in independent experiments). One-way and two-way analyses of variance (ANOVA) were used when comparing groups of conditions, while two-tailed unpaired non-parametric Mann-Whitney U tests or unpaired two-tailed t-tests were used when comparing pairs of conditions. Symbols used: * P≤0.05, ** P≤0.01, *** P≤0.001, **** P≤0.0001 and “ns” is non-significant.

### Example 2: Sequence-Dependent Effects of 2′OMe 2Gapmer ASOs on cGAS Sensing

cGAS has recently emerged as an essential sensor of cytosolic DNA deriving from pathogens and damaged endogenous nucleic acids (McWhirter and Jefferies, 2020). Upon activation by DNA, cGAS drives the formation of cyclic GMP-AMP (cGAMP), which binds to stimulator of interferon genes (STING) and promotes transcriptional induction of IRF3 responsive genes, including CXCL10 (IP-10) and IFNB1. Since it instigates deleterious immune responses linked to a wide range of diseases, various approaches are being investigated currently to therapeutically target cGAS (An et al., 2018; Laa et al., 2019; Padilla-Salinas et al., 2020; Vincent et al., 2017; Zhao et al., 2020). The present inventors originally hypothesised that strategies relying on the down-regulation of cGAS mRNA could provide other therapeutic avenues to those presented by chemical inhibitors of cGAS, which have been pursued in many other studies (An et al., 2018; Laa et al., 2019; Padilla-Salinas et al., 2020; Vincent et al., 2017; Zhao et al., 2020). To study this possibility, the present inventors designed a panel of 11 2′OMe gapmer ASOs (ASO1 to ASO11) that targeted the mRNA of human cGAS and tested their effects in HeLa cells and HT-29 cells which endogenously express cGAS (FIG. 1A and Table 1). Transfection of the ASOs led to a sequence-dependent decrease of cGAS mRNA, although variable between the two cell lines, with ASO2 having a 30-50% impact, while ASO3/ASO4 robustly failed to decrease cGAS expression (FIG. 1A).

The present inventors next tested the functional effect of there panel of ASOs on undifferentiated monocytic THP-1 cells, upon overnight uptake of the naked ASOs (FIG. 1B). In contrast to the down-regulation signature seen in HeLa and HT-29 cells, ASO2 was the most potent oligonucleotide blocking IP-10 production after transfection of synthetic 70-bp interferon stimulating DNA (ISD70) acting as cGAS ligand (Unterholzner et al., 2010). This observation was reproduced in cGAS/STING-competent epithelial HT-29 cells (Xia et al., 2016) where overnight incubation with 125 nM of ASO2 (but not ASO4, ASO10 or ASO11) blunted ISD70-induced IP-10 production (FIG. 1C). Critically, however, increased doses of ASO4, ASO10 and ASO 11 also blocked ISD70-induced IP-10 production, when used at 250 or 500 nM in HT-29 (FIG. 1C). Taking into account their differing effects on cGAS mRNA targeting (FIG. 1A), the dose-dependent inhibitory effect of ASO4/ASO10/ASO11 on ISD70-sensing strongly suggests it was independent of cGAS mRNA targeting, but rather related to a competitive effect on cGAS sensing of ISD70; this aligns with the effect observed for PS-ODN A151 on cGAS sensing (Steinhagen et al., 2018). The hypothesis that ASOs have a direct effect on cGAS function was also consistent with the observation that ASO11 increased ISD70-sensing at low doses, but was also inhibitory at higher doses, which may relate to the binding of the ASO to the third DNA binding domain of cGAS, increasing its enzymatic activity at low dose (Xie et al., 2019) (FIG. 1C).

Since ASO2 inhibited ISD70-sensing at a lower concentration than the other ASOs, it was posited that select motifs may increase the inhibitory effect of 2′OMe gapmer ASOs on ISD70-sensing of cGAS. To define this further, the present inventors screened a library of 80 2′OMe ASOs, designed to target the CDKN2B-AS1 and LINC-PINT transcripts (Table 2 and (Alharbi et al., 2020)), in THP-1 transfected with ISD70 (FIG. 1D). While none of these ASOs targeted the cGASmRNA, 23 out of 80 decreased ISD70-driven IP-10 production by more than 40% (FIG. 1D and Table 2). A few highly inhibitory ASOs were selected from the screen for validation in THP-1 (C2, E10 and F2 ASOs) and HT-29 cells (C2 and E10 ASOs) transfected with ISD70 (FIG. 1E, 1F). Overnight pre-treatment with C2 robustly inhibited ISD70-dependent IP-10 production in both cell models, while a significant effect of E10 was seen only in THP-1 cells (noting a reduction of 25% with this sequence in HT-29 cells). Importantly, C2 was a more potent inhibitor than ASO2 (FIG. 1E) and A151 (FIG. 1E, 1F).

The present inventors have recently shown that 2′OMe gapmer ASOs are spontaneously taken up by undifferentiated THP-1 and can modulate endosomal TLR7/8 sensing (Alharbi et al., 2020). To exclude a putative contribution of endosomal TLRs upon ISD70 transfection, the present inventors next tested the effect of C2 and ASO11 in THP-1 cells lacking cGAS (Mankan et al., 2014) or UNC93B1 (Pelka et al., 2014), the latter being devoid of TLR7/8 and 9 sensing. While cGAS-deficient and UNC93B1-deficient cells similarly produced IP-10 upon stimulation with a synthetic human STING agonist (Raanjulu et al., 2018), cGAS-deficient THP-1 cells also lacked responsiveness to ISD70 transfection (FIG. 1G). Conversely, the present inventors observed a dose-dependent inhibitory effect of C2 on ISD70 sensing in UNC93B1-deficient and matched wild-type cells, not seen with ASO11 (FIG. 1G), ruling out a contribution of endosomal TLRs in the effects of C2 on ISD70 sensing. Collectively these results demonstrate a capacity for 2′OMe gapmer ASOs to inhibit cGAS sensing of transfected DNA, in a sequence-dependent manner.

### Example 3: Identification of a 2′OMe Motif Controlling cGAS Inhibition

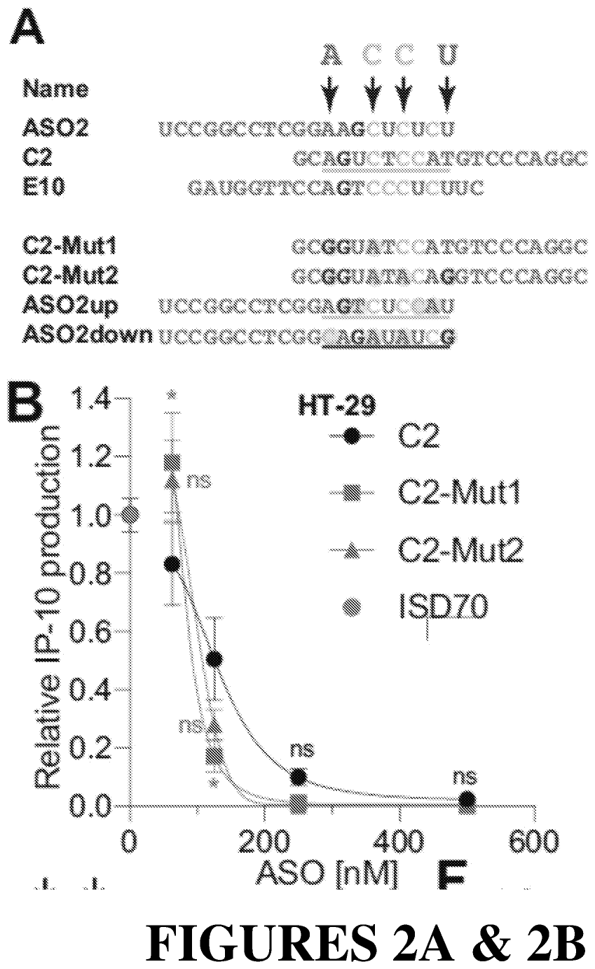

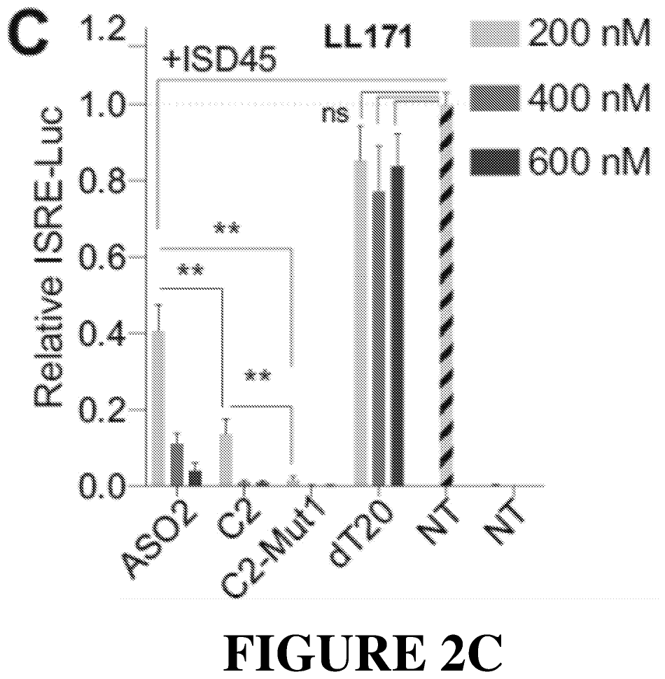

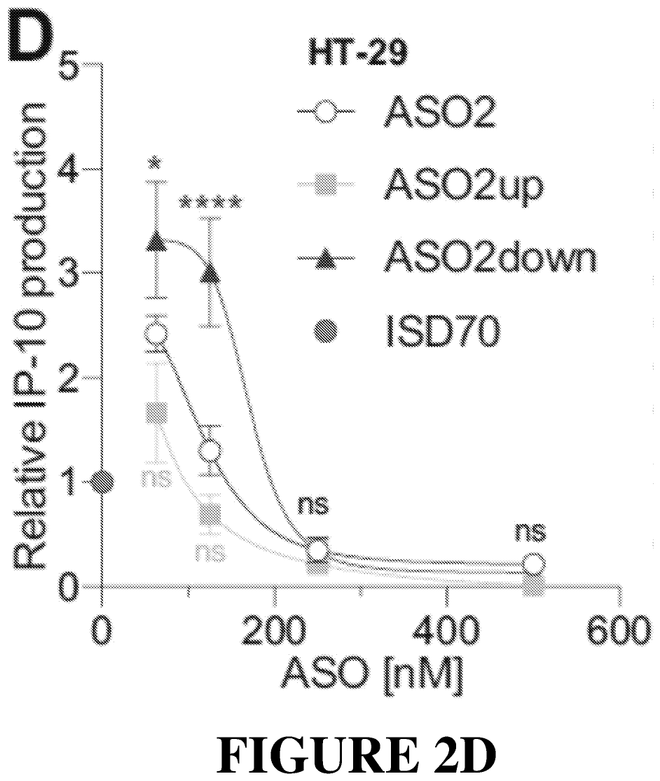

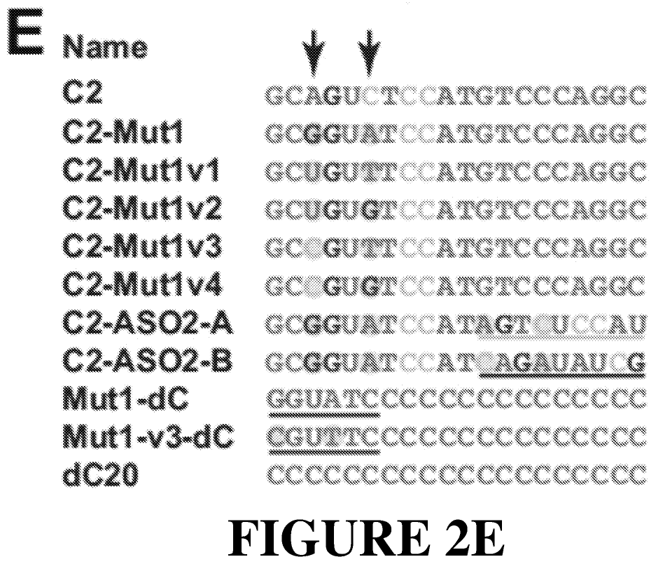

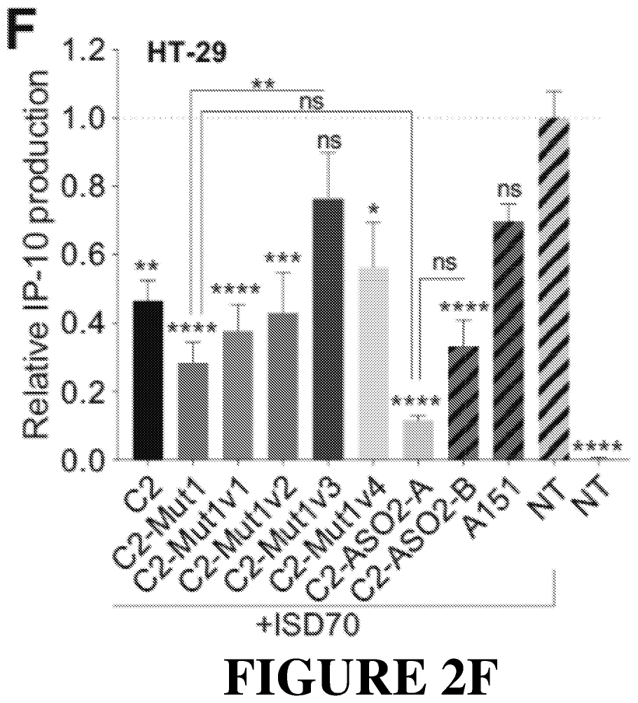

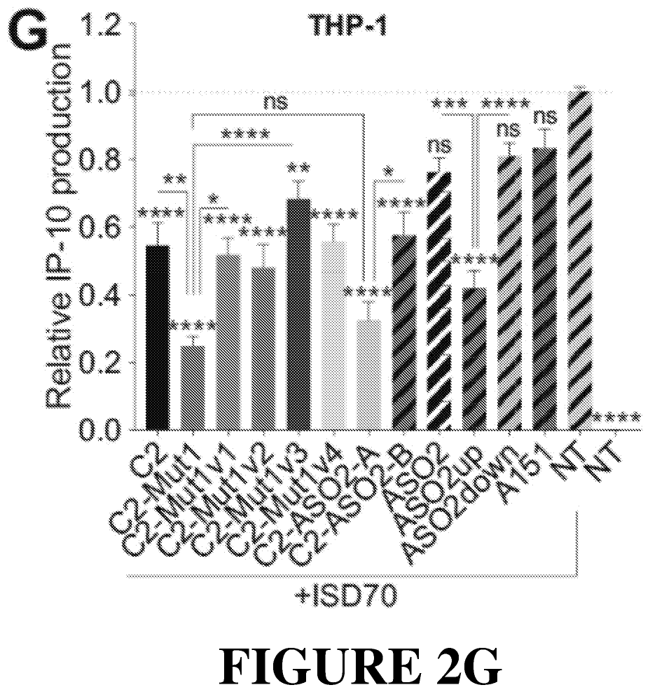

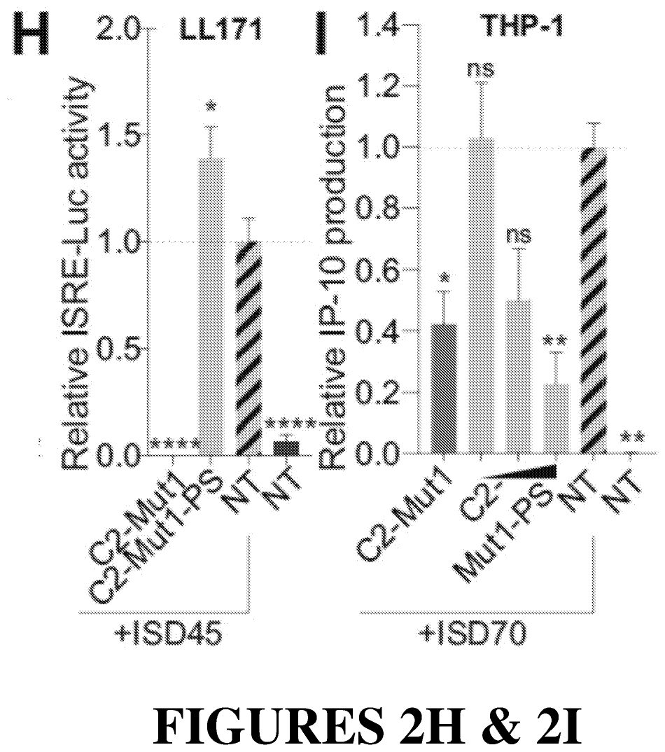

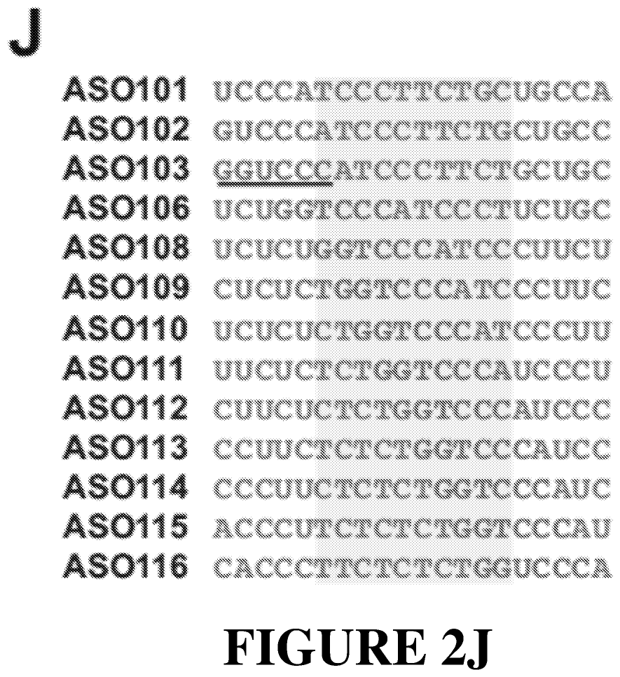

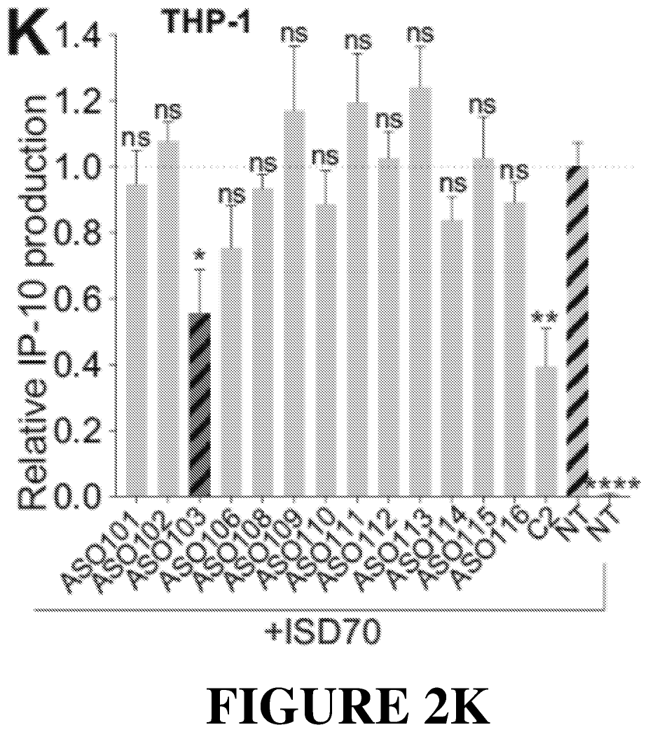

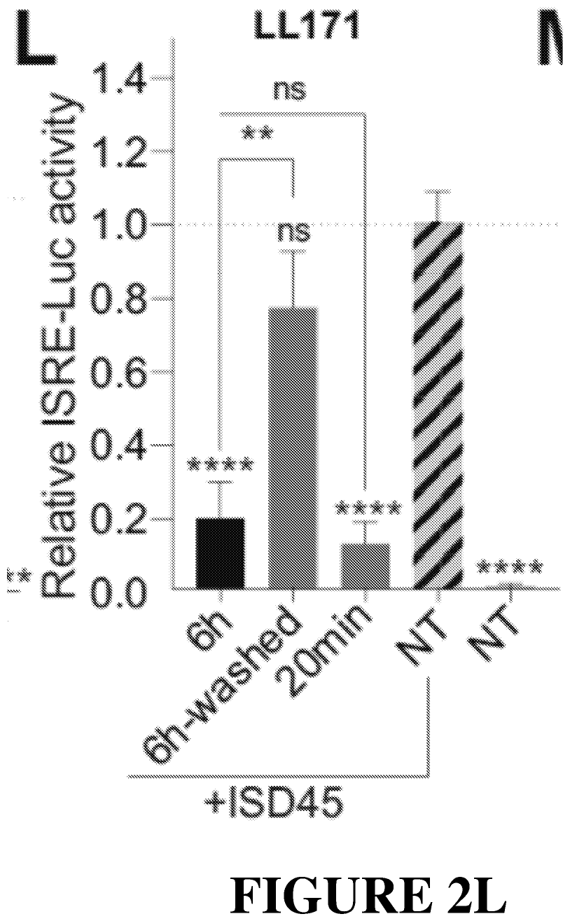

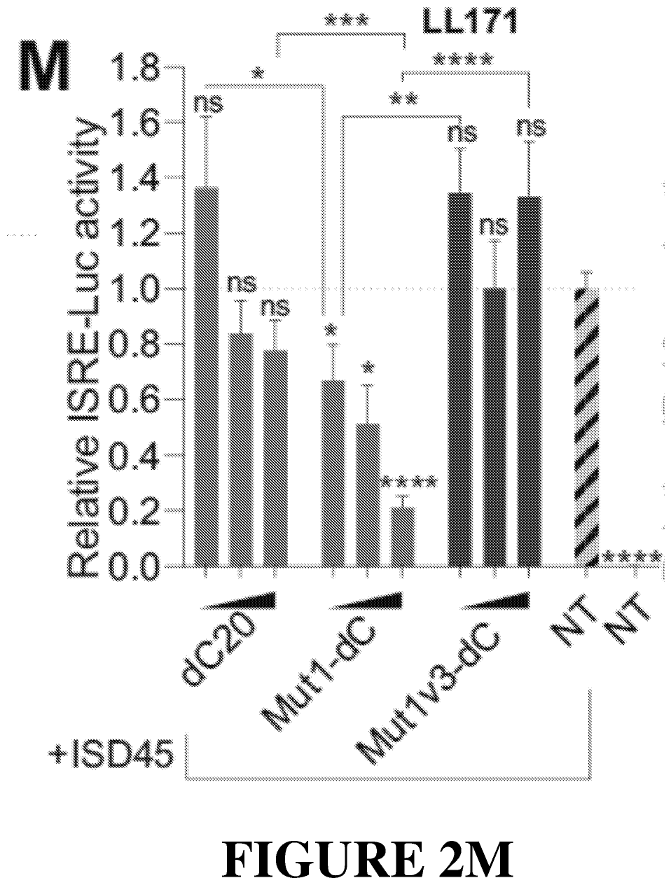

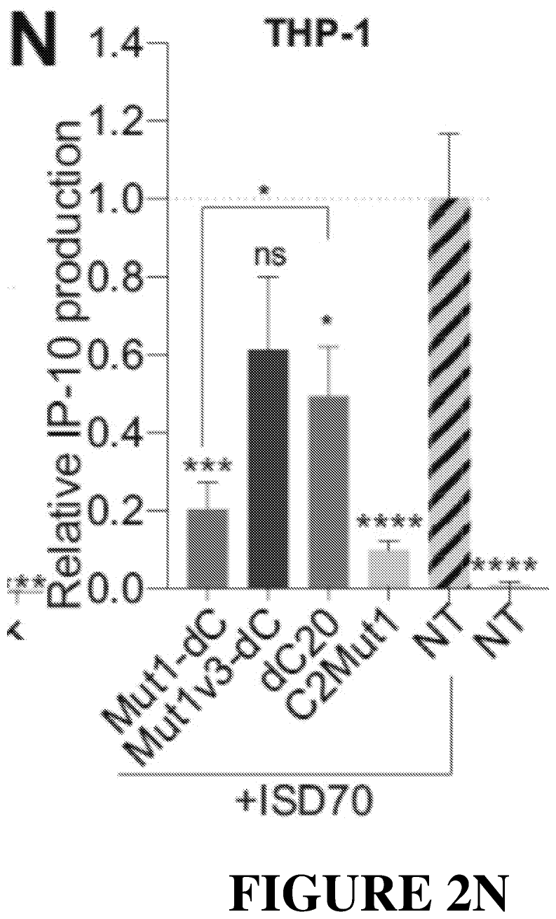

Having shown that ASO2, C2 and E10 were potent inhibitors of ISD70 sensing in THP-1, the present inventors next sought to define whether select motifs in these ASOs were involved in their inhibitory activities. MEME motif discovery analysis (Bailey and Elkan, 1994) performed on the three sequences identified a putative enriched motif in the 5′ half of C2 and 3′ half of both ASO2 and E10, with four highly conserved bases (FIG. 2A and FIG. 6A). To test the importance of these four bases, the present inventors first designed two variants of C2, substituting two (C2-Mutl) or four (C2-Mut2) of these bases (FIG. 2A)—noting that the bases used to replace those in the motif were arbitrarily chosen, and could equally decrease or increase the inhibitory activity. In accord with a direct contribution of these specific bases, dose-response analyses of the C2 mutants on ISD70-driven IP-10 production in HT-29 cells showed increased inhibitory activity for both ASOs at 250 and 125 nM compared to the parent C2 ASO (this decrease being significant for C2-Mutl at 125 nM; FIG. 2B). Similarly, C2-Mutl was significantly more inhibitory on ISD45-driven interferon stimulated response element (ISRE)-luciferase than C2 and ASO2 in mouse LL171 cells (FIG. 2C). Conversely in LL171 cells, the 20-mer PS oligonucleotide (dT20) did not inhibit ISD45 sensing even at 600 nM—confirming the sequence-specific nature of the ASO inhibition in mouse cells (FIG. 2C). In parallel, having shown that C2 was a more potent inhibitor than ASO2 (FIG. 1E), the present inventors designed mutants of ASO2 harbouring the 5′ region of C2 (ASO2up), or a mutant of the four conserved bases (ASO2down—FIG. 2A). While not significant in HT-29 cells, the trend was that ASO2up was a more potent inhibitor of ISD70-driven IP-10 production than ASO2 (at 125 nM); conversely, ASO2down significantly increased IP-10 production at 62.5 and 125 nM, aligning with what was seen with ASO 11 in HT-29 cells (FIG. 2D).

Since two base mutations of C2 in C2-Mutl significantly increased ISD70-driven IP-10 inhibition, the present inventors next designed a series of four C2-Mutl mutants (C2-Mutlv1 to C2-Mut1v4) with base permutations to define whether the inhibition seen with C2-Mutl could be improved further (FIG. 2E). The present inventors also designed hybrid ASOs fusing the 5′ half of C2-Mutl to the 3′ half of ASO2up (C2-ASO2-A) or ASO2down (C2-ASO2-B). Analyses of these sequences in HT-29 (FIG. 2F) and THP-1 (FIG. 2G) revealed that C2-Mutl was robustly the most inhibitory sequence of ISD70 sensing, and was significantly more potent than its mutant C2-Mut1v3. Interestingly, C2-ASO2-A, which contains two fused inhibitory motifs (that of C2-Mutl in the 5′ half, and that of ASO2up/C2 in the 3′ half), had a similar potency to that of C2-Mut1, indicating that duplicating the inhibitory motif in the 3′ end region of the ASO did not significantly improve the inhibition (FIGS. 2F, 2G). Nonetheless, in THP-1 cells, C2-ASO2-B was significantly less inhibitory than C2-ASO2-A and ASO2down was significantly less inhibitory than ASO2up, confirming that 3′ end regions could also play a role in the inhibition of ISD70 sensing (FIG. 2G).

### Example 4: cGAS Inhibition by a Minimal mGmGmUATC Motif

The present inventors next asked whether the 2′OMe chemical modification of gapmer ASOs was at play in their effect on cGAS. The inhibitory effect of C2-Mutl was significantly decreased in a C2-Mutl analogue where the 2′OMe ends were replaced by DNA bases on a PS-backbone (referred to as C2-Mutl-PS), in both mouse LL171 and human THP-1 cells (FIGS. 2H, 2I and Table 1). In addition, MEME motif discovery on the 10 most inhibitory ASOs from the screen (FIG. 1D and Table 2) revealed that 5 ASOs harboured a conserved [A/G]GUC[U/C]C (SEQ ID NO: 344) motif aligned with that of C2 and C2-Mut1, which was predominantly overlapping with their 2′OMe 5′ end (FIG. 6B). Interestingly, one of these sequences, ‘[LINC-PINT] 103’ (referred to as ASO103), was part of a family of sequences with single base increments (Table 2 and FIG. 2J). Within this series, ASO103 was the only sequence significantly inhibiting ISD70-driven IP-10 production in THP-1 cells, further supporting that the 5′-end location and 2′OMe modifications were likely essential to the ASO inhibitory function (FIG. 2K).

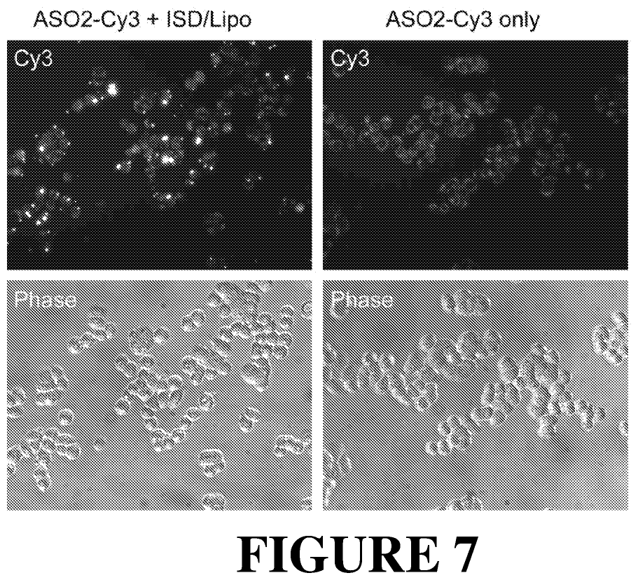

The data collected so far demonstrated that long (i.e. 6 h-16 h) pre-incubation with 2′OMe gapmer led to inhibition of ISD sensing by cGAS. To tease out the role of the pre-incubation on the inhibitory activity of the ASOs, the present inventors compared the effect of C2-Mutl pre-incubated for 6 h in LL171 cells, with or without a wash prior to the transfection of ISD45. Also tested was the effect of ASOs added a short time prior to the transfection of ISD45 (˜20 min). While pre-incubation of the ASOs for a shorter duration did not impact their inhibitory effect on ISD45-driven ISRE-Luc expression, the addition of a washing step significantly blunted inhibition (FIG. 2L). These findings suggested that the ASOs were somewhat co-transfected with the liposome containing ISD. Accordingly, increased intracellular fluorescent punctua of ASO2-Cy3 were observed following ISD70 transfection of HT-29 cells (FIG. 7), altogether suggesting that the ASOs competed with ISD for cGAS sensing intracellularly.

Relying on this shorter ASO pre-incubation, the present inventors next sought to confirm the core motif modulating cGAS inhibition in C2-Mutl, starting from a 20-mer homopolymeric sequence of dCs on a PS backbone (dC20), to which the minimal 2′OMe containing terminal 5′ mGmGmUATC motif of C2-Mut1 was added (referred to Mutl-dC) (FIG. 2E). Mutl-dC was significantly more inhibitory than its precursor dC20 on ISD sensing in LL171 and THP-1 cells (FIGS. 2M, 2N). In addition, the two base-mutations (mCmGmUTTC) in Mutlv3-dC lacked significant inhibitory activity in both cell models (FIGS. 2M, 2N). Collectively with previous observations, these results establish that the minimal mGmGmUATC motif is sufficient to confer cGAS inhibition to a PS-modified homopolymeric sequence, with a predominant role for the two bases previously identified.

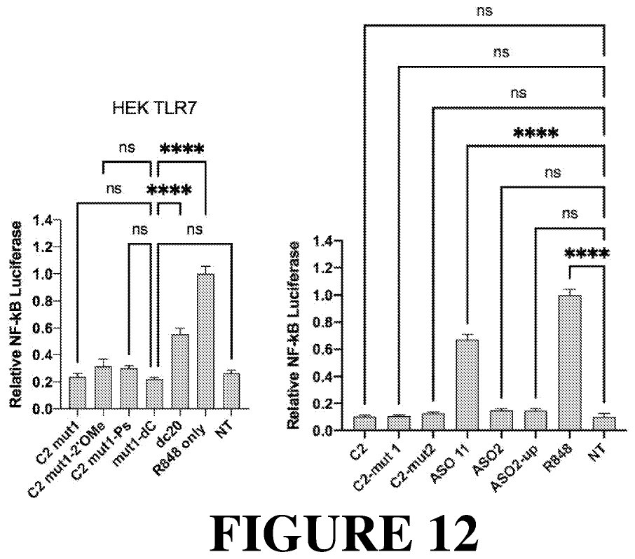

The present inventors further found that Mut-ldC, which harbours the cGAS inhibitory motif also demonstrates significantly higher TLR7 inhibition than dC20 (FIG. 12). This establishes that the minimal motif presently defined for cGAS is also capable of conferring TLR7 inhibition to oligonucleotides.

### Example 5: Sequence-Specific Inhibition of cGAS Activity by C2-Mutl

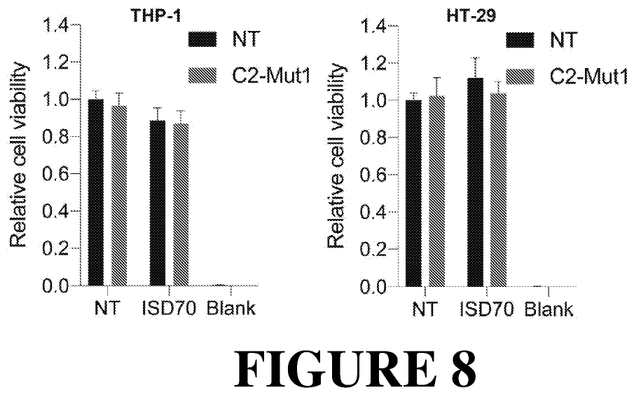

Having identified C2-Mutl as the most potent 2′OMe ASO inhibitor of cGAS, the present inventors performed dose-response analyses to determine its IC50 in the THP-1 model and to compare it to that of A151 (Steinhagen et al., 2018). Based on IP-driven production after ISD70 transfection, it was determined that the IC50 of C2-Mut1 was 56 nM compared to 165 nM for A151 (FIG. 3A). This greater inhibitory activity of C2-Mutl over that of A151 on DNA sensing was confirmed looking at the production of IFN-P, which was blocked with C2-Mutl at 125 nM, while only reduced by ˜50% for A151 (FIG. 3B). It is also noteworthy that treatment of the cells with C2-Mut1 did not significantly impact cell viability in the assays in THP-1 and HT-29 cells (FIG. 8).

Critically, 6 h pre-incubation with high dose C2-Mutl (500 nM) did not significantly affect IP-10 production driven by lipopolysaccharide (LPS-TLR4 ligand) or STING synthetic agonists, in human MG-63 and immortalised mouse bone marrow derived macrophages (BMDMs), although significantly impacting ISD sensing (FIGS. 3C, 3D). Similarly, C2-Mutl did not impact production of TNF-α upon LPS, PAM3CSK4 (TLR2/1 ligand) or DMXAA (mouse Sting agonist) treatment (FIG. 3D). Nonetheless, C2-Mutl pre-incubation significantly decreased sensing of the CpG ODN 1826 which activates mouse Tlr9, as revealed by dampened TNF-α production in immortalised BMDMs (FIG. 3D). This is consistent with the previous report that PS-2′OMe ASO can impact endosomal sensing by TLRs such as TLR7/8 in phagocytes (Alharbi et al., 2020), but indicates that C2-Mutl is not broadly inhibiting non-nucleic acid sensing pathways such as TLR1/2/4, nor acting at the level of downstream signalling cascades.

In vitro, cGAS has previously been shown to be bound and weakly activated by single stranded ISD45 (Kranzusch et al., 2013), leading us to posit that single stranded PS-ASOs could act as “inactive” competitors of double stranded ISD. To directly assess this, recombinant cGAS was incubated in vitro with 2.3 μM (0.1 μg/l) of ISD70 in the presence of increasing amount of C2-Mutl or A151 (0.5, 2 and 10 μM). Critically, while both PS-ODNs similarly reduced cGAMP production driven by ISD70 at 0.5 μM, the inhibitory activity of C2-Mutl was significantly greater than that of A151 (about 2 fold more) at 2 μM (FIG. 3E). Under these specific conditions in vitro, C2-Mutl was therefore able to displace more molecules of ISD70 than A151, suggesting its stronger affinity for cGAS than A151. Collectively with the cell based assays, these observations support that C2-Mutl acts as an inactive competitor of ISD70 binding to cGAS.

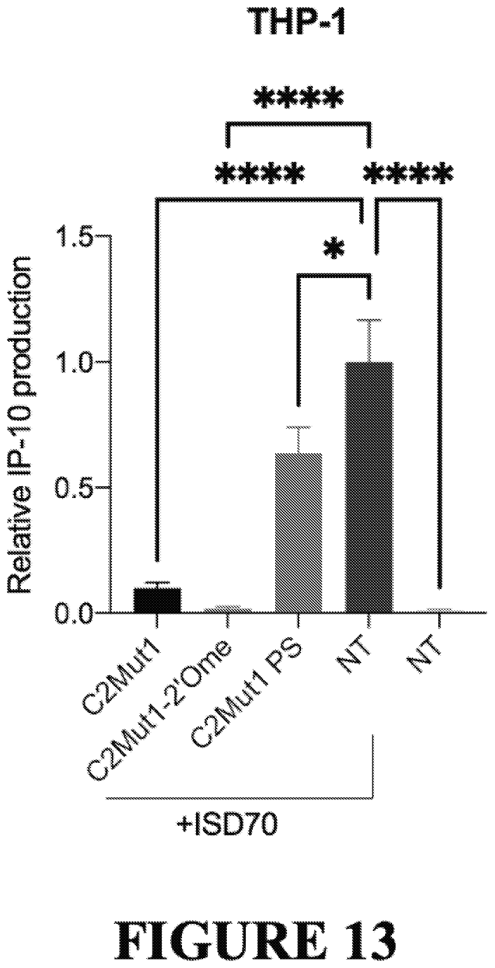

The present inventors further demonstrated that a fully 2′Ome modified version of C2Mut1 was also strongly blunting of cGAS activation by TSD70 (FIG. 13).

### Example 6: C2-Mut1 Inhibits Constitutively Active cGAS Signalling

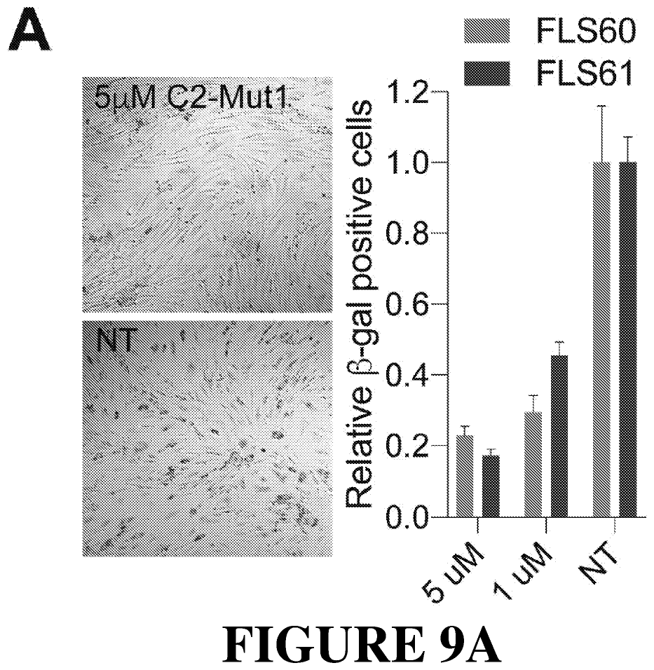

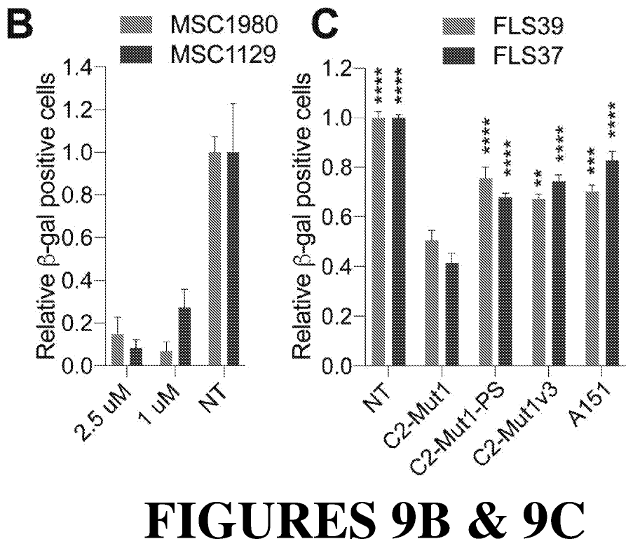

Since the experiments to this point strictly relied on cGAS sensing of exogenously transfected ISD, the present inventors next investigated the effects of the ASOs on cell models with constitutive cGAS activation. They first compared the dose-dependent effect of C2-Mutl and C2-Mut1v3 transfected In human BJ hTERT fibroblasts stably expressing SV40T and RASG12V (Quin et al., 2016), which display a basal level of constitutive cGAS activation (Pepin et al., 2017). Transfected C2-Mutl was significantly more potent than its two nucleotide variant C2-Mut1v3 in inhibiting expression of several interferon stimulated genes (ISGs) (IFIT1, IFIT2 and IFI44) constitutively expressed in these cells (Uhlen et al., 2017), while being comparable to aspirin treatment that blocks cGAS activity (Dai et al., 2019) (FIG. 3F). Secondly, overnight transfection of C2-Mutl and C2-Mutl-v3 significantly decreased the basal expression of two ISGs (Rsad2 and Ifit1) in primary mouse bone marrow derived macrophages (BMDMs) from Trex1-mutant mice (Q169×—see Material and Methods)—which exhibit accumulated cytoplasmic DNA and constitutive cGAS-STING signalling (FIG. 3G) (McWhirter and Jefferies, 2020). Conversely, transfection of a similar dose of A151 failed to inhibit these ISGs (FIG. 3G). cGAS activation has recently been shown to play an important role in the paracrine propagation of senescence between cells (Gluck et al., 2017; Dou et al., 2017). As such, senescence-associated β-galactosidase (SAB) was strongly decreased during the expansion of primary mouse fibroblasts from cGas-deficient mice (Gluck et al., 2017). the present inventors therefore decided to investigate the capacity of C2-Mutl to inhibit spontaneous engagement of cGAS-STING signalling, in pre-senescent primary fibroblasts-like synoviocytes (FLS) and primary bone marrow-derived mesenchymal stem cells (MSCs). For this purpose they grew the primary cells in the presence of 1 to 5 μM C2-Mutl ASO (passively taken up by the cells by gymnosis) for one to two weeks. While a large proportion of cells were senescent in non-ASO treated cells after this period (based on β-galactosidase positive staining—seen in up to ˜40-50% of the cells), senescence was strongly reduced in C2-Mutl-treated FLS cells and MSCs (FIG. 9A, 9B). Further experiments in another set of FLS cells showed that C2-Mutl was more potent in inhibiting SAB than its PS only variant C2-Mutl-PS, or its mutant C2-Mutlv3. Similarly, C2-Mutl was a more potent inhibitor of SAB than A151 (FIG. 9C), aligning with the concept that this activity pertained to its increased inhibition of cGAS.

Collectively, these results demonstrate that C2-Mutl significantly inhibits constitutive cGAS sensing of endogenous cytoplasmic DNA, in a sequence-dependent manner.

### Example 7: Sequence-Dependent TLR9 Inhibition by 2′OMe Gapmer ASOs

Having demonstrated the sequence-dependent modulation of cGAS sensing of DNA by 2′OMe gapmer ASOs, the present inventors next turned to their effect on DNA sensing by TLR9, with the aim of providing a broad picture of their impact on DNA sensing in human cells. As such, while there is ample evidence for sequence-dependent TLR9 modulation with select PS-ASOs (Krieg et al., 1995; Barrat et al., 2005), including A151 (Gursel et al., 2003), the impact of 2′OMe moieties in the context of PS-gapmer ASOs, is not defined. They recently demonstrated that 2′OMe gapmer ASOs did not frequently activate TLR9, with the exception of “T” rich sequences (Alharbi et al., 2020). To characterise whether 2′OMe gapmer ASOs instead led to inhibition of DNA sensing by TLR9, as indicated with the results in BMDMs with C2-Mut1 (FIG. 3D), the present inventors tested the panel of 11 cGAS ASOs on TLR9 sensing of CpG ODN2006 in HEK cells expressing human TLR9 (HEK-TLR9 hereafter) and a NF-κB luciferase reporter. They included PS-ODN A151 and IRS957, which have been reported to inhibit TLR9, along with their respective controls, C151 and IRS661 (Gursel et al., 2003; Barrat et al., 2005)). Several ASOs significantly inhibited TLR9 sensing of ODN2006, including ASO2, 4, 5, 6, 7, 8, 9, 10 (FIG. 4A). However, ASO2, IRS957 and A151 were the only oligonucleotides reducing NF-κB luciferase induction by more than 80%. The previous observation that ASO11, which lacked the capacity to inhibit TLR9 but strongly potentiated TLR8 in HEK cells (Alharbi et al., 2020), indicated that the sequence-specific effects seen here were not related to uptake of the ASOs, but rather due a competitive effect of specific motifs on ODN2006 sensing by TLR9.

To investigate further the sequence determinants of TLR9 inhibition by 2′OMe gapmer ASOs, the present inventors initially assessed the effect of ASO2 3′-end mutants (ASO2up and ASO2down) and ASO 11 mutants where the 5′ or 3′ 2′OMe regions had been swapped with those of ASO2 (ASO1 IMut1 and ASO11Mut2) in HEK-TLR9 cells (FIGS. 4B, 4C). The four-base substitutions in the 3′-end of ASO2down significantly reduced TLR9 inhibition, unlike those in ASO2up, indicating the inhibitory effect was partially dependent on the sequence of the 3′ half of ASO2. Critically, substitution of ASO11 5′-end 2′OMe region with that of ASO2 (ASO11Mut2), but not its 3′-end (ASO1 IMut1), conferred significant TLR9 inhibition to ASO11, suggesting that the 5′-end of ASO2 is also at play in its regulatory effect on TLR9 (FIG. 4C).

To gain further insights into the sequence-dependent inhibition of TLR9 sensing of DNA by 2′OMe gapmer ASOs, the present inventors next tested the panel of 80 2′OMe ASOs in HEK-TLR9 cells, (FIG. 4D and Table 2). They observed that only 8 out of the 80 ASOs inhibited TLR9 by more than 50% (Table 2), and ASO2 remained the most potent ASO tested. MEME motif discovery on the top 10 ASOs from this screen revealed significant enrichment in 3 ASOs of a “GGCCTC” (SEQ ID NO: 204) motif also present in ASO2, and a “A”-rich central region in 5 out of the 10 ASOs (FIGS. 4E, 4F and FIGS. 6C, 6D). Accordingly, comparison of the central DNA regions of the 16 most and least inhibitory ASOs in the TLR9 screen showed a significantly higher proportion of central “As” in more potent TLR9 inhibitory sequences (FIG. 4G and Table 2).

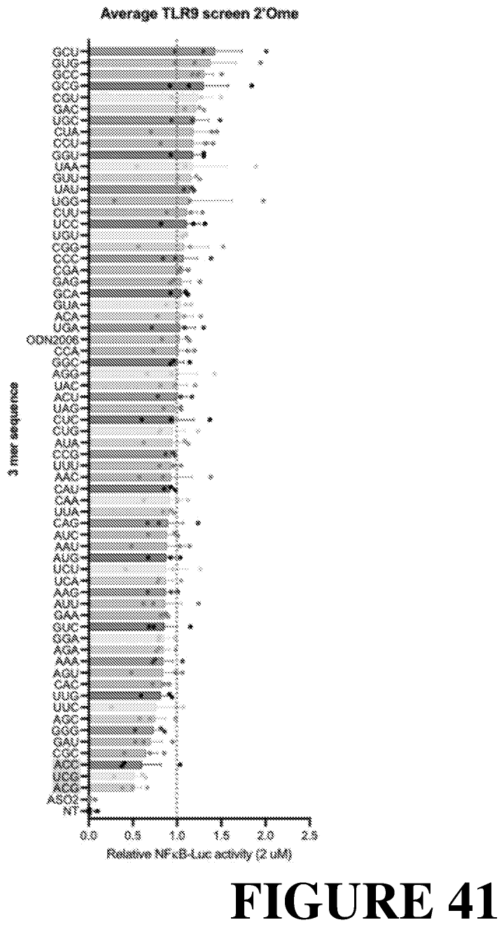

Closer analyses of two ASOs families from the screen with single base increments also pointed to an important role for 5′ 2′OMe “CUU” (SEQ ID NO: 153) motifs in TLR9 inhibition (Table 2). Validation of the first family, referred to as the “CDKN2B-AS1” series (ASO2133-2139), suggested that addition of a 5′-end terminal “CUU” motif in ASO2139 significantly increased TLR9 inhibition (FIG. 4H, 4I). Aligning with this observation, analyses of the “LINC-PINT” series of ASOs (ASO8-ASO116), also harbouring 5′ or 3′ “CUU” (SEQ ID NO: 153) motifs, demonstrated that “CUU/CUT” motifs (CUU-SEQ ID NO: 153; CUT-SEQ ID NO: 202) present in the 2′OMe region of the ASOs (ASO112-115) were associated with a significant increase in TLR9 inhibition compared to ASO108-110 containing such 2′OMe “CUU” (SEQ ID NO: 153) motifs in their 5′ end (FIGS. 41, 4J).

To define further the role of 2′OMe “CUU” (SEQ ID NO: 153) motifs on the modulation of TLR9 sensing, the present inventors next used an additional panel of 2′OMe ASOs targeting the mRNA of HPRT (FIG. 4K and (Alharbi et al., 2020)). While 5′-end terminal “CUU” (SEQ ID NO: 153) motifs in ASO [HPRT]551 and 660 were associated with >60% inhibition of TLR9, addition of a single 5′-end terminal base to the “CUU” (SEQ ID NO: 153) motif (“ACUU”, SEQ ID NO: 346) in ASO661 significantly limited inhibition (FIG. 4K, 4L). Conversely, AS0662, with its “CACUUU” (SEQ ID NO: 347) 5′-end 2′OMe region displayed the strongest inhibition of this series of ASOs, although this may also be related to a contribution of its “UUUUC” (SEQ ID NO: 348) 3′-end (uniquely present in this sequence in this series). Interestingly, AS0666 also strongly inhibited TLR9, while not displaying any “CUU” (SEQ ID NO: 153) motif and only differing by two bases from AS0665, which was only poorly inhibitory (FIG. 4K, 4L). Nonetheless these results are only suggestive of a role for selected motifs in the modulation of TLR9 inhibition, and need to be taken with caution given the putative impact of base increments on 2′OMe regions, which could confound results interpretation. Taken together, these results demonstrate a complex regulation of TLR9 inhibition by 2′OMe gapmer ASOs, including preferential contributions from central (preferentially “A” rich) and 5′-end 2′OMe regions.

In contrast with these findings, the present inventors have recently reported that 2′OMe “CUU” (“CUU”, SEQ ID NO: 153) motifs could help mitigate TLR7 inhibition by 2′OMe gapmer ASOs (Alharbi et al., 2020), suggesting an inverse relationship between the modulation of these two sensors. They therefore analysed the correlation of the effect of the 80 ASOs on TLR9 and TLR7 inhibition (Alharbi et al., 2020), presented in FIG. 4M. There was no correlation between the activities of the ASOs on TLR7 and TLR9, exemplified with ASO[LINC-PINT]108, ASO109, ASO116 and A10 which had limited effect on TLR9, and TLR7, unlike ASO[CDKN2B-AS1]2139, which did not inhibit TLR7 but strongly inhibited TLR9.

### Example 8: C2-Mutl is a Potent Inhibitor of Human cGAS, TLR7 and TLR3 but not TLR9

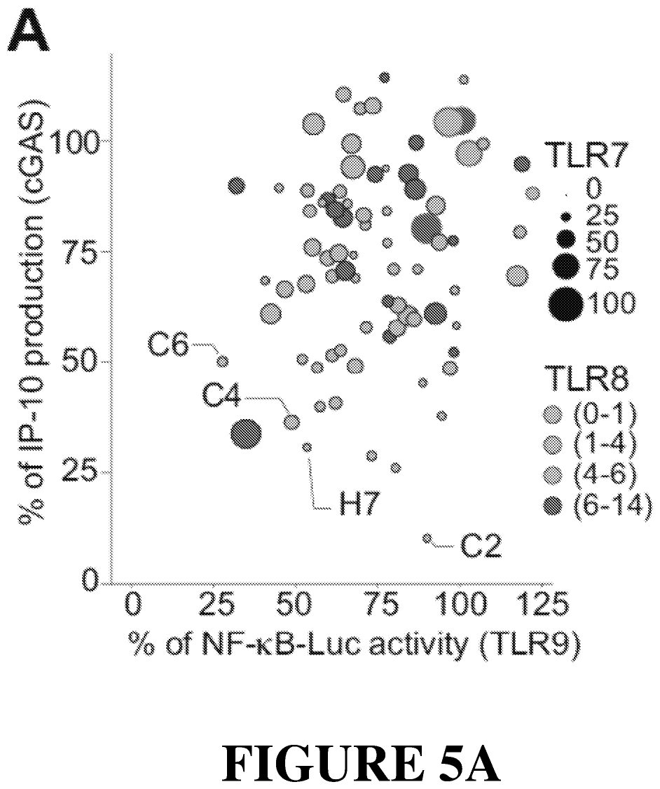

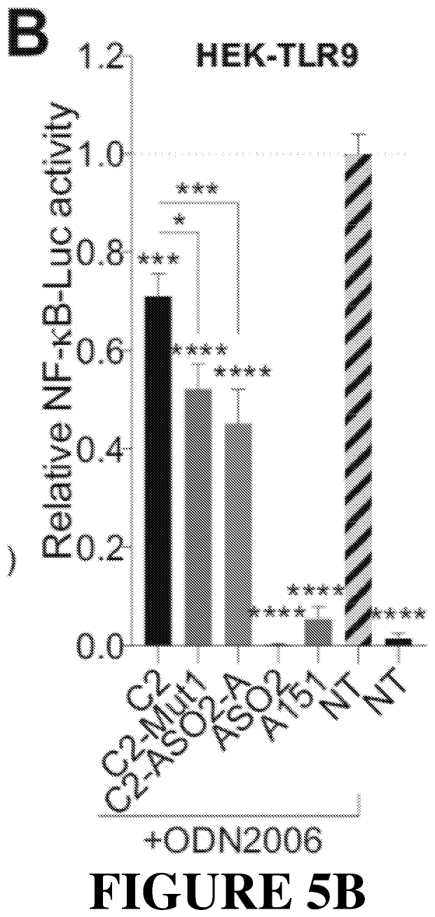

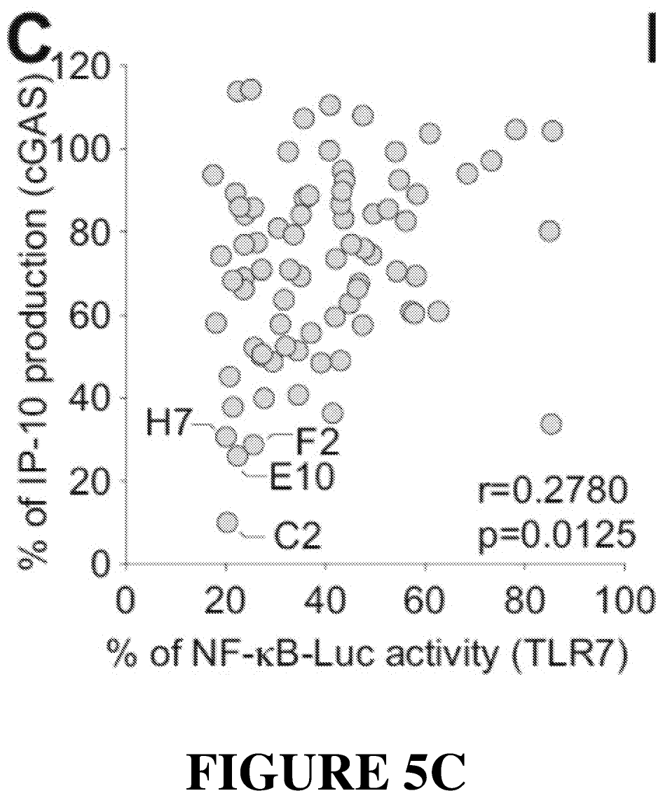

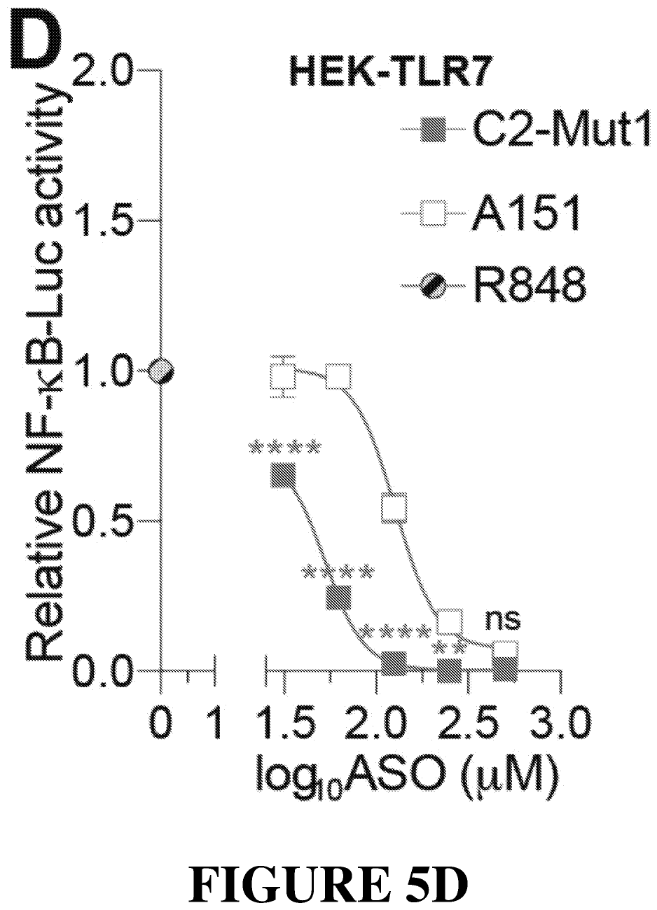

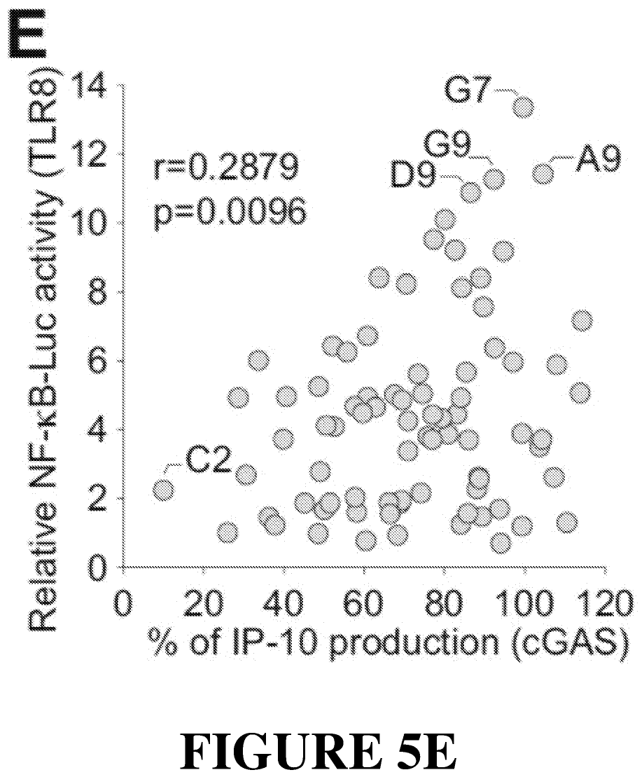

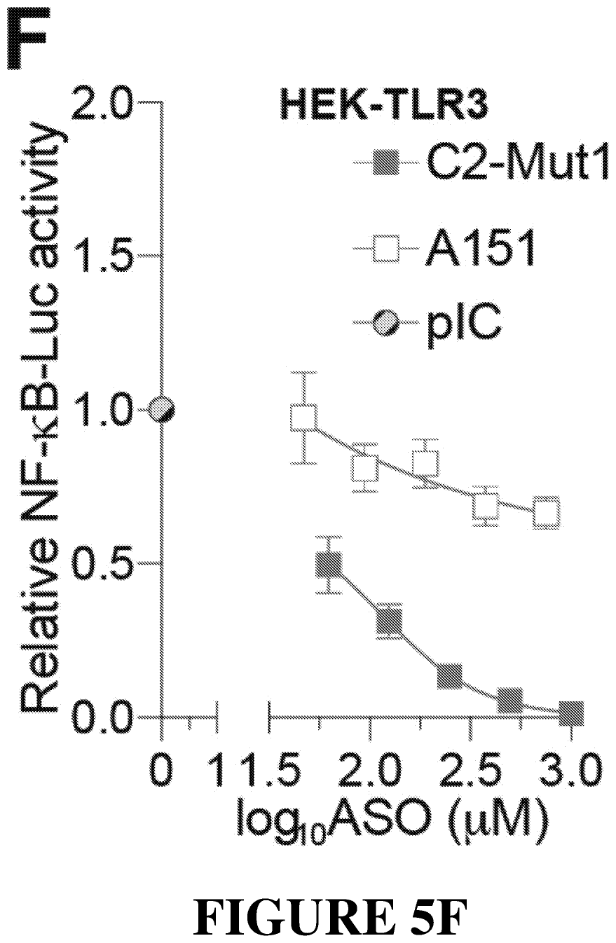

Having generated immunomodulatory profiles for the panel of 80 2′OMe ASOs on TLR7/TLR8 (Alharbi et al., 2020), TLR9 and cGAS, the present inventors generated a bubble chart to visualize the correlation of inhibition of DNA sensing by cGAS and TLR9, while also incorporating data on TLR7 inhibition and TLR8 potentiation (FIG. 5A and Table 2). There was no significant correlation between TLR9 and cGAS inhibition, illustrated by the example that C2, the most potent cGAS inhibitor in the screen did not inhibit TLR9 (FIG. 5A). This observation was validated in HEK-TLR9 cells with C2 and its mutants C2-Mutl and C2-ASO2-A. While the C2 mutants were significantly better inhibitors of TLR9 than their parent ASO, their inhibitory effect on TLR9 sensing was limited compared to that of ASO2 or A151 (FIG. 5B).

As shown in FIG. 5A, the patterns of immunomodulation on these sensors was highly variable with only a few ASOs inhibiting TLR7, TLR9 and cGAS (e.g. C4, C6 and H7) (Table 2). Nonetheless, there was a significant correlation between cGAS and TLR7 inhibition (FIG. 5C), with the strongest inhibitors of cGAS also displaying strong TLR7 repression (e.g. C2, E10, F2, H7; FIG. 5C). Consistent with this, the most potent cGAS inhibitor, C2-Mutl, was also a strong inhibitor of R848 sensing by TLR7, with an IC50 of 44 nM, against 132 nM for A151 (FIG. 5D). Critically, only 4 ASOs in the screen (i.e. 5%) did not inhibit any of TLR7, TLR9 or cGAS by more than 30% (highlighted in red in FIG. 5A, and in yellow in Table 2), demonstrating that most 2′OMe ASOs have immunosuppressive effects.

Unlike their inhibitory effect on TLR7, the present inventors recently discovered that select 2′OMe gapmer ASOs could potentiate TLR8 sensing of R848, presenting potential therapeutic opportunities in immune-oncology (Alharbi et al., 2020). Interestingly, TLR8 potentiation was also inversely correlated with cGAS inhibition, with the best TLR8 potentiators lacking cGAS inhibition (e.g. G7, A9, D9, G9) (FIG. 5E). Accordingly C2 was a weak potentiator of TLR8, but select ASOs may be able to inhibit cGAS while potentiating TLR8 sensing, as seen with the example of ASO2 (FIG. 1 and (Alharbi et al., 2020)).

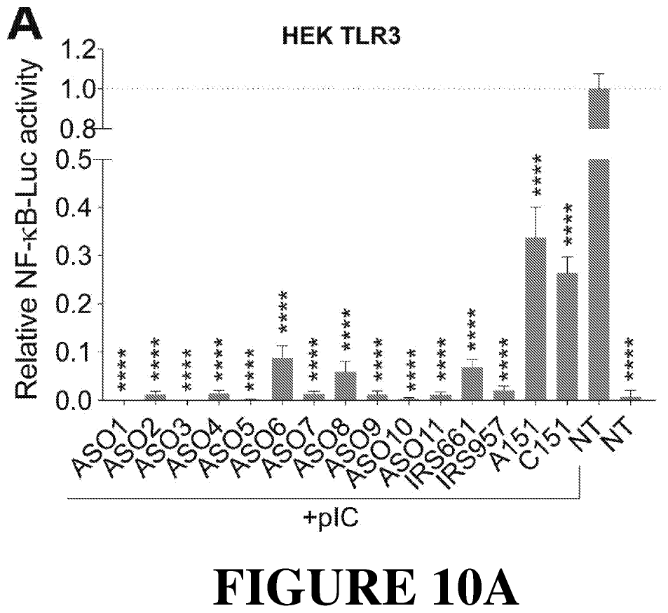

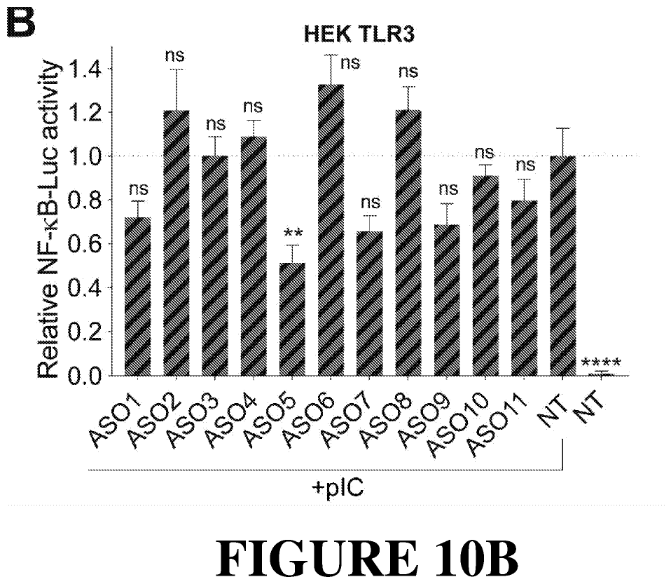

Finally, the present inventors tested the effect of the panel of 11 cGAS ASOs on the sensing of untransfected double stranded RNA (polyL:C) by human TLR3 sensing (including IRS661, IRS957, A151 and its mutant C151) in HEK 293 cells stably expressing human TLR3 (HEK-TLR3) and a NF-κB luciferase reporter (noting that these cells are not responsive to the amount of untransfected polyI:C they used, through RIG-I or MDA5). While all 2′OMe gapmer ASOs used at 500 nM along with IRS661 and IRS957 blocked polyL:C sensing, A151 and C151 only partially reduced polyL:C-dependent TLR3 activation in these cells (FIG. 10A). Sequence-specific inhibition of TLR3 was more visible with 100 nM ASOs, where only ASO5 retained significant inhibition of polyL:C sensing (FIG. 10B). Direct comparison of the inhibitory effect of C2-Mutl and A151 on TLR3 sensing in HEK-TLR3 cells confirmed the stronger inhibition of C2-Mut1, with an IC50 of 62 nM for C2-Mutl and an IC50 greater than 750 nM for A151 (FIG. 5F). Collectively these results demonstrate that C2-Mutl is a potent inhibitor of human cGAS, TLR7 and TLR3, but a weaker inhibitor of TLR9.

### Example 9: Motif-specific inhibition of TLR7 by 2′O-methyl ASOs

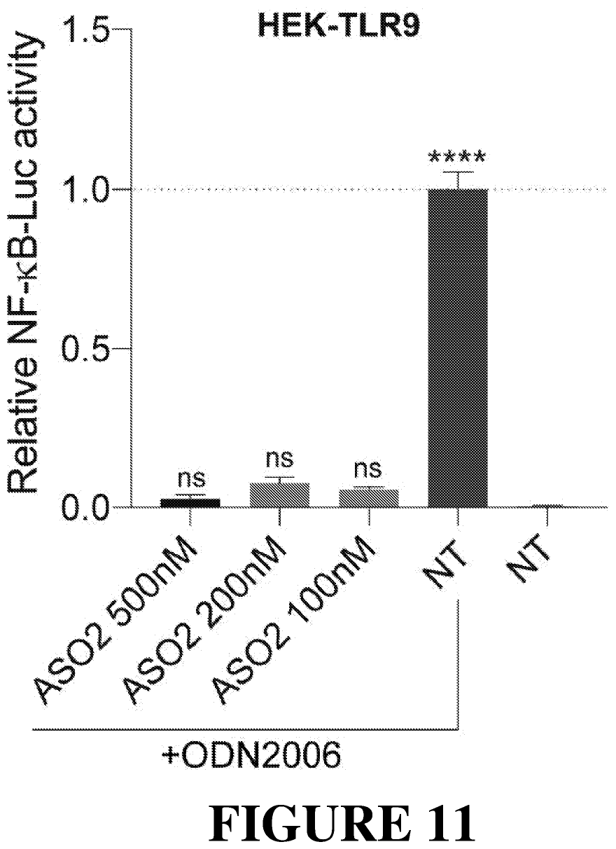

Previous Examples have shown that addition of the mG*mG*mU*A*T motif to a stretch of dCs on a phosphorothioate backbone was sufficient to promote inhibition of R848 sensing by TLR7 (see FIG. 11, Mutl-dC compared to dC20 ASO), directly implicating the 2′O-methyl (2′OMe) motif in this inhibitory effect.

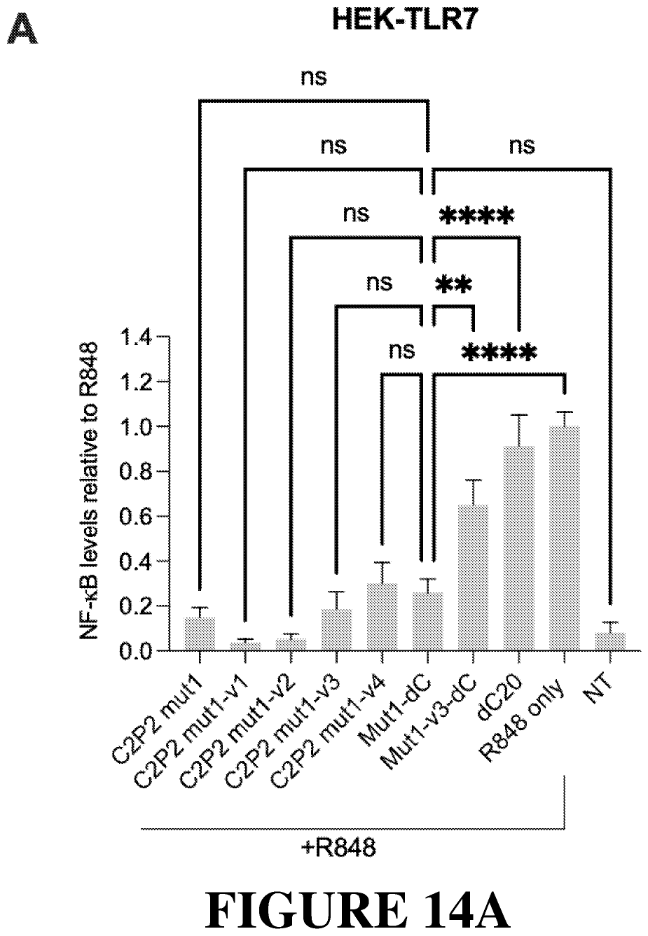

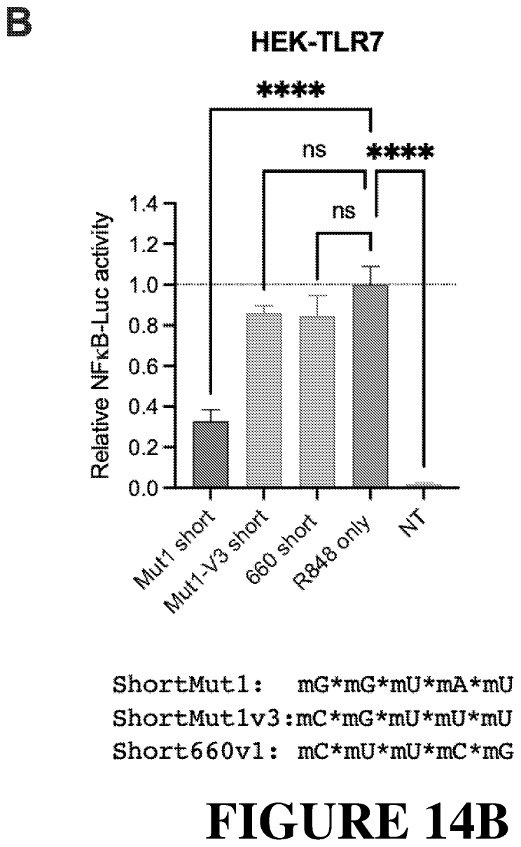

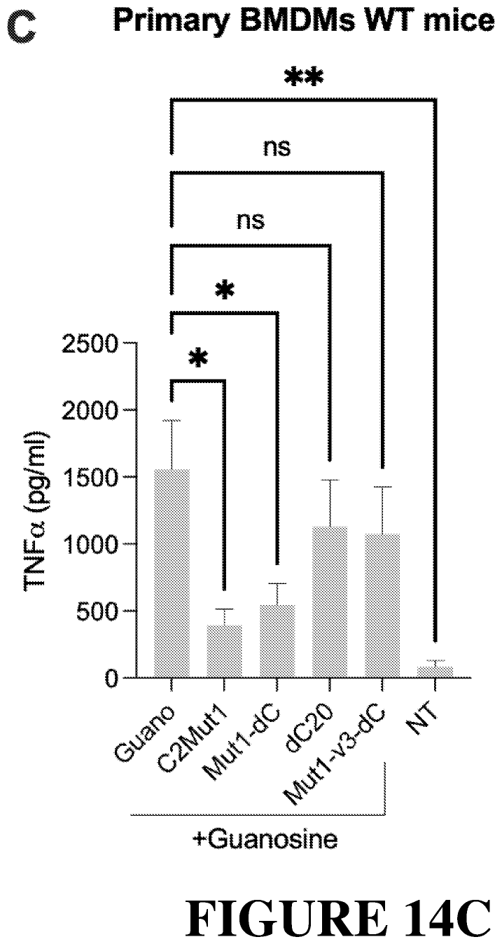

Critically, it is now found that substitution of two bases in this motif significantly altered TLR7 inhibition (compare Mutl-dC and Mutl-v3-dC), demonstrating the motif-specific effect of mG*mG*mU*A*T on TLR7 inhibition in HEK TLR7 cells (FIG. 14A).

Further, it is hypothesized that similar to TLR8 sensing of RNA (Greulich et al., 2019), the ASOs may be cleaved at selective positions by a yet-to-be-defined enzyme, to release the TLR7 inhibitory motif To test this, 5-nt short 2′OMe oligonucleotides were synthesized with a full PS-backbone containing the Mut1 motif and its variant Mut1-v3 (Mutl-short and Mutl-v3-short, respectively). The present inventors also included a short oligonucleotide which was not expected to inhibit TLR7 (based on ASO 660). Accordingly, it has been demonstrated that Mutl-short was sufficient to inhibit R848 sensing in HEK-TLR7 cells, while its variant and unrelated sequence failed to do so (FIG. 14B). It is noteworthy that pre-incubation of 6 h was necessary to obtain inhibition with Mutl-short and no inhibition was observed with −30 min pre-treatment. Collectively, these observations clearly demonstrate that TLR7 inhibition can be achieved by the specific “mGmGmUmAmU” motif.

### Example 10: Motif Specific Inhibition of Guanosine Sensing by 2′O-Methyl ASOs

To broaden the present observations, the present inventors next tested whether the 2′OMe ASOs could inhibit TLR7 activation by Guanosine which acts as an endogenous TLR7 ligand (Shibata et al., 2016). For this purpose, the effect of the ASOs was tested on primary bone marrow derived macrophages (BMDMs) from wild-type mice, stimulated overnight with 500 μM Guanosine and pre-treated or not with 200 nM ASOs. Similar to what was seen with inhibition of R848 sensing by TLR7 in HEK TLR7 cells, the ASOs inhibited TNFα production induced by guanosine, in a sequence specific manner—with C2-Mutl and Mutl-dC significantly inhibiting TNFα levels while Mutl-v3-dC or dC20 did not (FIG. 14C).

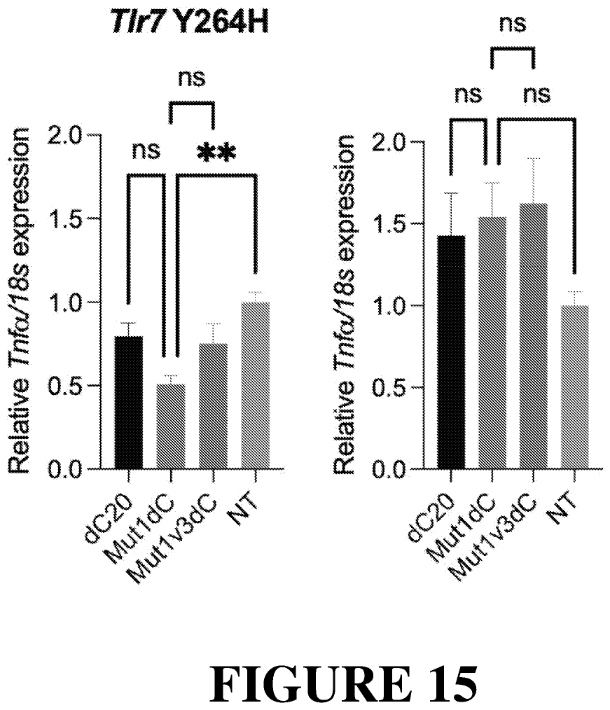

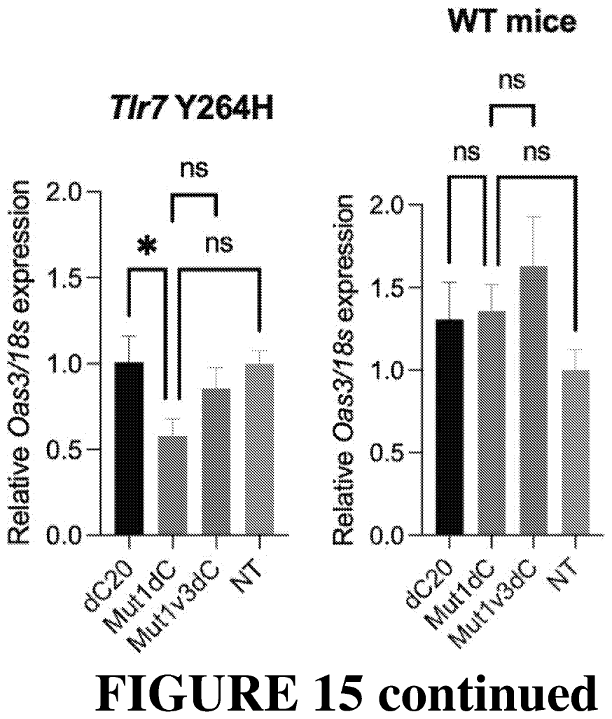

In accordance with this, Mutl-dC but not its mutant Mutl-v3-dC significantly reduced constitutive TLR7 activation in primary BMDMs from Tlr7 mutant mice (Tlr7 Y264H—Brown and Vinuesa, et al. Nature, 2022, in press), as measured with reduced TNFα and Oas3 mRNA levels. Importantly, the effect of Mutl-dC was specific to TLR7 since the ASOs did not reduce TNFα and Oas3 mRNA levels in wild-type primary BMDMs (FIG. 15).

Collectively, these results provided direct evidence that while most 2′OMe ASOs are strong TLR7 inhibitors (excluding those with a “CUU” motif that limits inhibition), selected motifs conferred a stronger inhibition of TLR7 sensing than others. The observation that the mG*mG*mU*mA*mU (SEQ ID NO: 56) of Mutl-dC and Mut1-short inhibits TLR7 sensing of guanosine but not its mC*mG*mU*mU*mU (SEQ ID NO: 349) variant, establishes a very specific activity of selected residues in the motif conferring inhibition.

Beyond the widespread inhibitory effect on TLR7 by ASOs, the present work establishes the capacity of selective short oligonucleotide motifs to act as strong TLR7 antagonists. This challenges the previous report that TLR7 inhibition could be achieved by any 2′OMe-U, 2′OMe-G, or 2′OMe-A modified RNAs with no sequence-dependent effect (Robbins et al., 2007). The use of short 5-mer oligos such as Mutl-short could therefore present novel opportunities to limit TLR7 engagement when combined to unmodified T7-synthesised RNA used in mRNA vaccines, as an alternative to the use of uridine modifications (such as pseudo-uridine) of the mRNA itself.

### Example 11: Motif-Specific Inhibition of TLR7 is not Restricted to 2′OMe ASOs

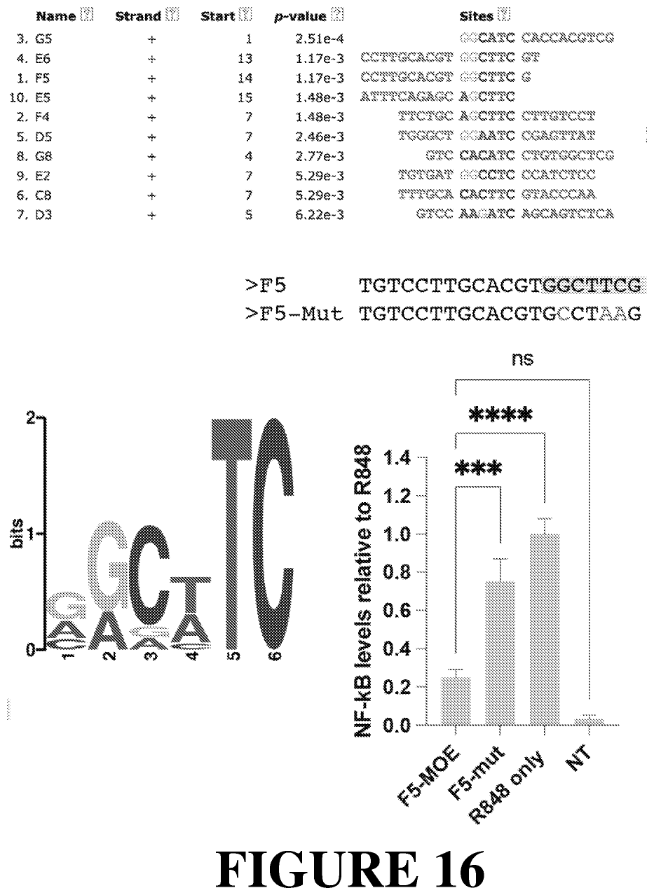

Previous Examples have screened 91 LNA and 76 2′MOE modified ASOs for TLR7 inhibition in HEK TLR7 cells (see PCT 2020901606). While searching for motifs that may underpin TLR7 inhibition, the present inventors identified a selective GGCTTC (SEQ ID NO: 295) motif enriched in the top 10 2′MOE TLR7 inhibitors from this screen (FIG. 16). More specifically, positions 2, 5 and 6 of this motif (i.e. xGxxTC) were very enriched. To confirm the involvement of this motif in TLR7 inhibition the present inventors chose one of these sequences, F5 (EGFR-1014 MOE), and mutated its predicted motif at positions 2, 5 and 6 (FIG. 16). While F5 was strongly inhibiting TLR7, its mutant F5-Mut was significantly less inhibitory directly demonstrating the role of this motif in TLR7 inhibition. These results demonstrate that the 2′MOE GGCTCC (SEQ ID NO: 295) motif in F5 is directly implicated in TLR7 inhibition, establishing that motif specific TLR7 inhibition is not limited to 2′OMe ASOs and can also be observed with other chemical modifications.

### Example 12: Inhibition of cGAS by 2′O-methyl modified ASOs

Previous Examples have demonstrated that 2′OMe ASOs with a 5′ end mG*mG*mU*A*T motif are potent inhibitors of cGAS (Valentin et al., 2021). Critically, this motif was sufficient to confer inhibition of cGAS to the 5′enf of a stretch of 15 dCs (effect which was ablated when the motif was mutated to mC*mG*mU*T*T directly implicating these two bases in the effect). To determine whether this approach could be used to turn any 2′OMe ASO into a cGAS inhibitor, the present inventors next assessed whether adding bases to the 5′end of an ASO targeted to the mRNA of HPRT [ASO 847] (with very potent gene targeting efficacy—see (Alharbi et al., 2020)) could increase its cGAS inhibitory activity. Critically, the 5′end of AS0847 is mA*mU, meaning that only mG*mG*mU was appended to its 5′end to reconstitute the mG*mG*mU*mA*mU motif (giving a 23 nt ASO-847-Mut).

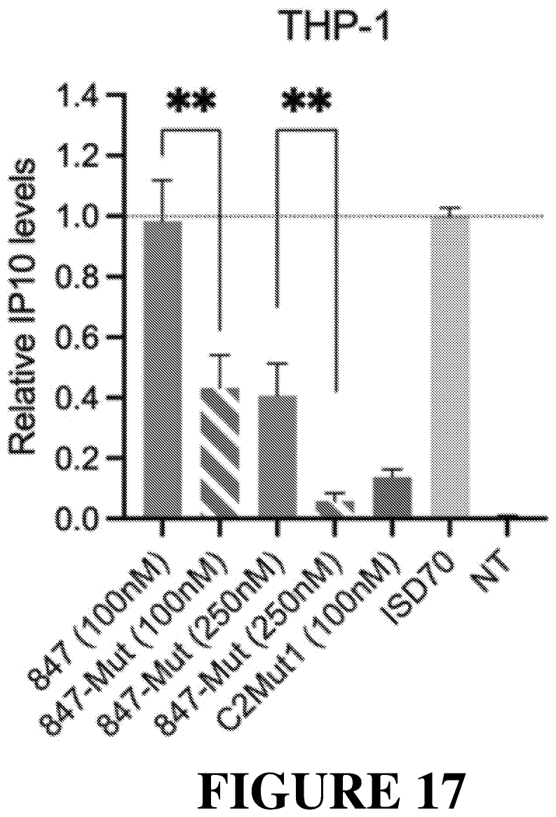

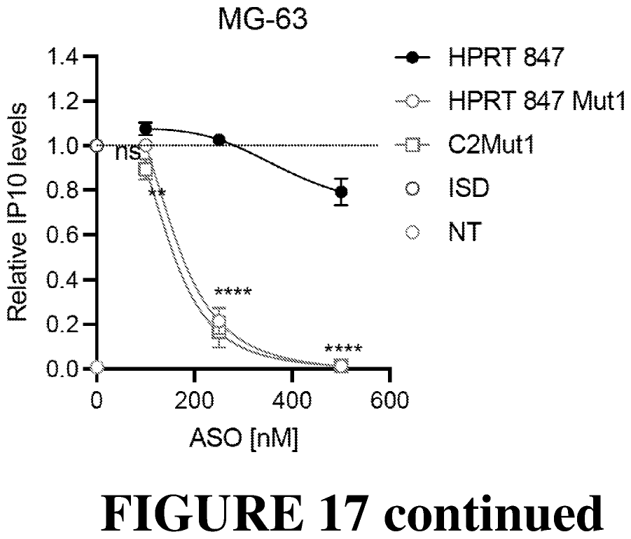

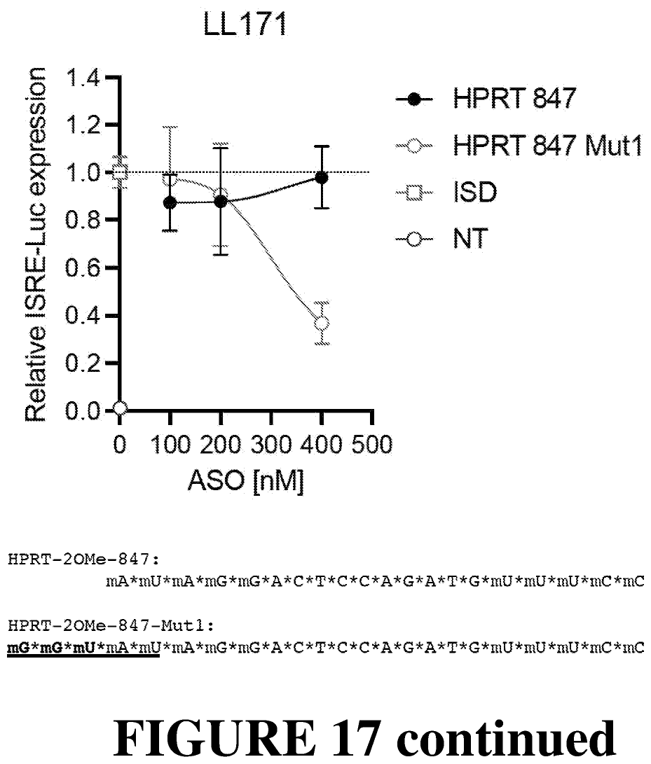

Accordingly, the present inventors tested the inhibitory effect of AS0847 and ASO847-Mut in THP-1 and MG-63 cells transfected with the cGAS ligand ISD70 (FIG. 17). In both models it was demonstrated that ASO847-Mut was a much better inhibitor of cGAS—directly demonstrating proof-of-principle that adding a few 5′end nucleotides to reconstitute the inhibitory motif of C2-Mutl (Valentin et al., 2021) was sufficient to increase cGAS inhibition of an otherwise poorly inhibitory sequence. Similarly, ASO847-Mut was a better inhibitor against mouse cGAS activation by ISD45 in LL171 cells.

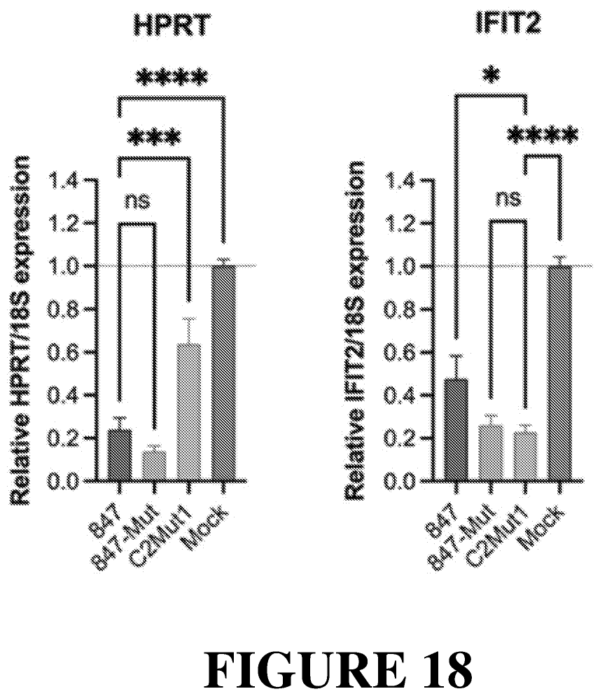

To further these observations it was tested whether ASO847-Mut retained its inhibitory activity against HPRT targeting, while inhibiting constitutive ISG expression in BJ7 cells (human fibroblasts expressing SV40T) (Valentin et al., 2021). These experiments confirmed that ASO847-Mut was still able to reduce HPRT levels, to the level seen with AS0847 (FIG. 18). Critically however, ASO847-Mut was as potent as C2-Mutl to decrease expressing of the ISG IFIT2 in these studies, while AS0847 was significantly less potent—aligning with the experiments conducted in MG-63 cells (FIG. 17).

Collectively, these experiments established proof of principle that a pre-existing ASO could be modified through 5′ end base additions to reconstitute the 5′ mG*mG*mU*mA/A*mU/T cGAS inhibitor motif, to confer increased cGAS inhibition.

The present inventors have previously shown that the mG*mG*mU*A*T cGAS inhibitor motif, appended to the 5′ end of a 15 bases dC oligonucleotide (Mutl-dC), significantly increased cGAS inhibition in a motif-dependent manner—since the 2 base mutant Mutl-v3-dC did not (4). Mutl-v3-dC contains mutations at position 1 and 4 of the motif (mC*mG*mU*T*T) establishing that at least one of these bases is essential for cGAS inhibition.

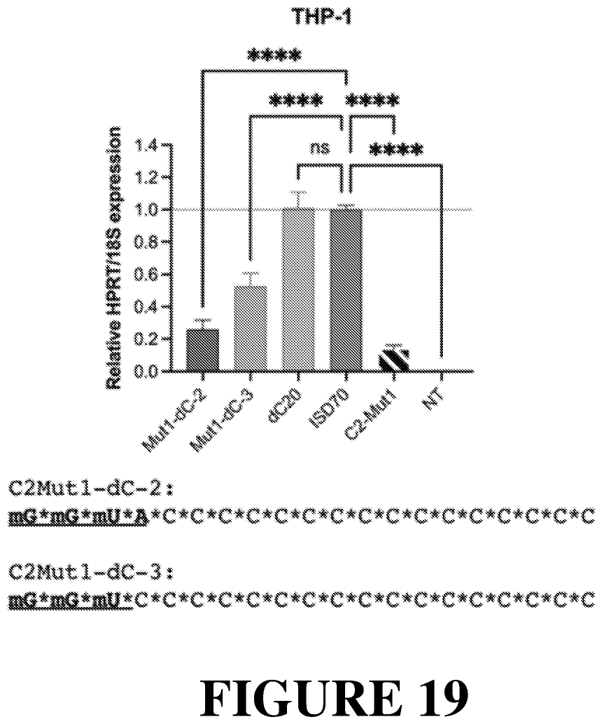

In order to define whether the mG*mG*mU*A*T cGAS inhibitor motif could be truncated further, the present inventors generated two shorter variants of Mutl-dC, referred to as Mutl-dC-v2 (mG*mG*mU*A) and Mutl-dC-v3 (mG*mG*mU) the latter lacking base #4 of the motif. Analyses in THP-1 cells stimulated with the cGAS ligand ISD70 demonstrated that both shorter forms significantly inhibited cGAS sensing, but that the 4-mer was more potent than the 3-mer motif (FIG. 19). These results suggest that 5′ additions to an ASO reconstituting a 5′ mG*mG*mU or mG*mG*mU*A/mG*mG*mU*mA motif are sufficient to increase its capacity to inhibit cGAS.

### Example 13: Inhibition of cGAS by 2′MOE and LNA Modified ASOs

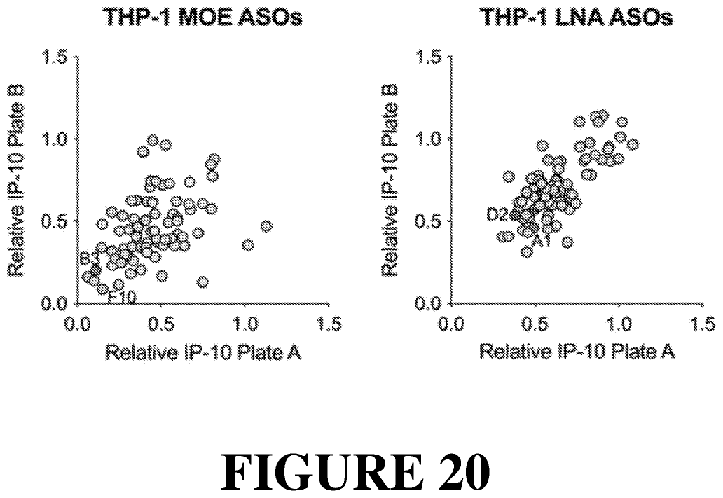

To define whether cGAS inhibition could be promoted by other ASO chemical modifications, 87 LNA and 76 2′MOE modified ASOs were screened for reduction of IP-10 production upon ISD70 transfection of THP-1 cells. While selected ASOs that repressed cGAS sensing with either chemistry, the overall repression appeared stronger with 2MOE ASOs than LNA ASOs. As such, 32/87 (i.e. 36.7%) ASOs repressed cGAS signal by more than 40% with LNA, versus 59/76 (i.e. 77.6%) for 2MOE at the dose used (FIG. 20).

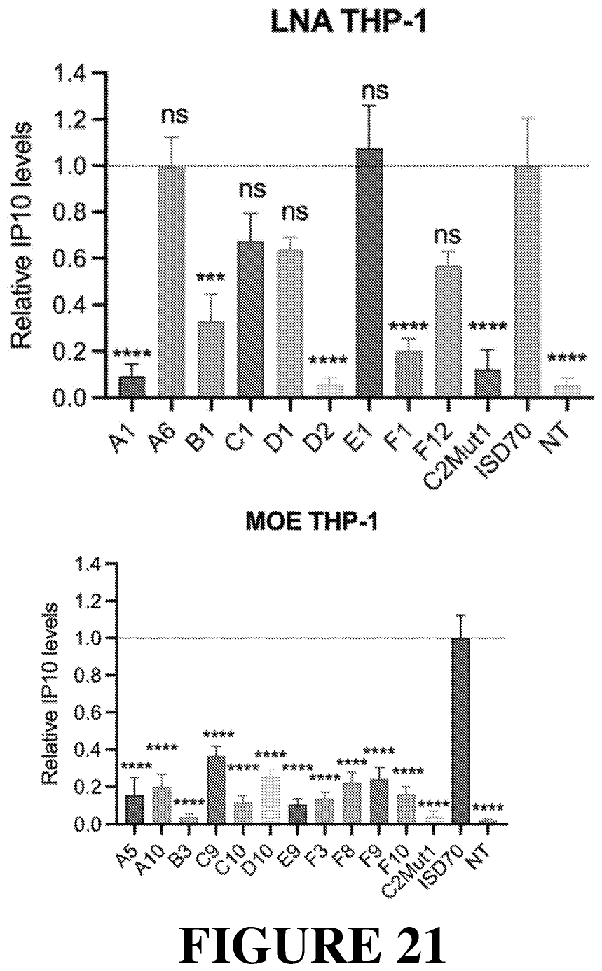

The present inventors next selected the top 9 inhibiting ASOs for the LNA screen. In agreement with a lower inhibitory effect of LNA ASOs, several of the ASOs failed to significantly inhibit cGAS in validations experiments (FIGS. 21—e.g. A6 and E1)-noting that 300 nM ASOs was used here, compared to 200 nM for MOE ASOs after. Nonetheless, select ASOs potently inhibited signalling, with A1, D2 and F1 being the most potent (FIG. 21).

For the MOE screen, the present inventors selected the top 11 inhibiting ASOs. Aligning with their better inhibitory activity than LNA ASOs, all the MOE ASOs tested in these validation experiments significantly inhibited cGAS, with B3 and E9 being the most potent (FIG. 21).

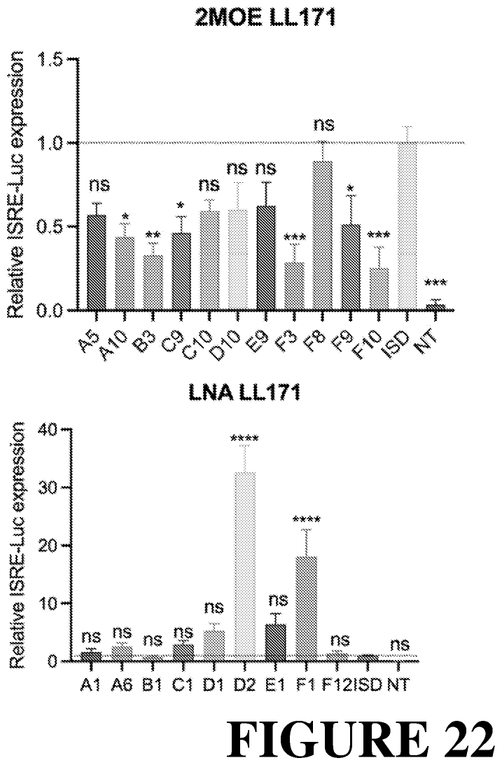

The present inventors also wanted to test whether the ASOs could inhibit murine cGAS—since it was found that some 2′OMe ASOs such as C2-Mutl could be active in both species. The present inventors therefore tested the 9 LNA ASOs and 11 MOE ASOs from the screen and assessed them for inhibition of ISD sensing in LL171 reporter cells. For MOE ASOs, half the sequences significantly inhibited cGAS in this system, with B3, F3, F10 being the most potent (FIG. 22).

Conversely, at the dose used, little to no inhibition was observed with the LNA ASOs, with two ASOs strongly potentiating the ISRE-Luciferase signal in response to transfected ISD—D2 and F1 (FIG. 22). While surprising, the present inventors have previously observed a significant potentiation of cGAS with selected ASOs (Valentin et al., 2021), and it has recently been reported that selected CpG oligonucleotides could directly engage with cGAS to activate it (Bode et al., 2021). It was particularly noteworthy that D2 would potentiate cGAS signalling in mouse while being one of the few significant inhibitors of cGAS in human cells—highlighting key differences of ASO effects on cGAS between species.

### Example 14: Motif-Specific Inhibition of cGAS by 2′MOE and LNA Modified ASOs

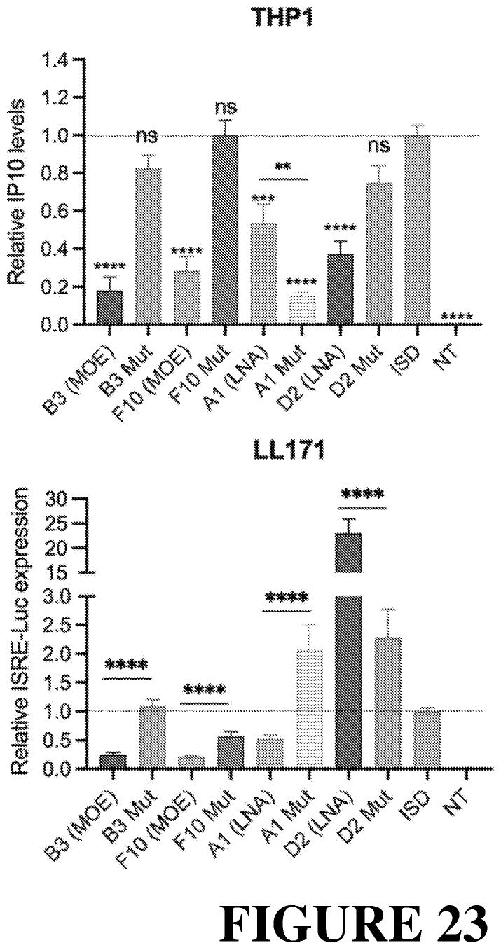

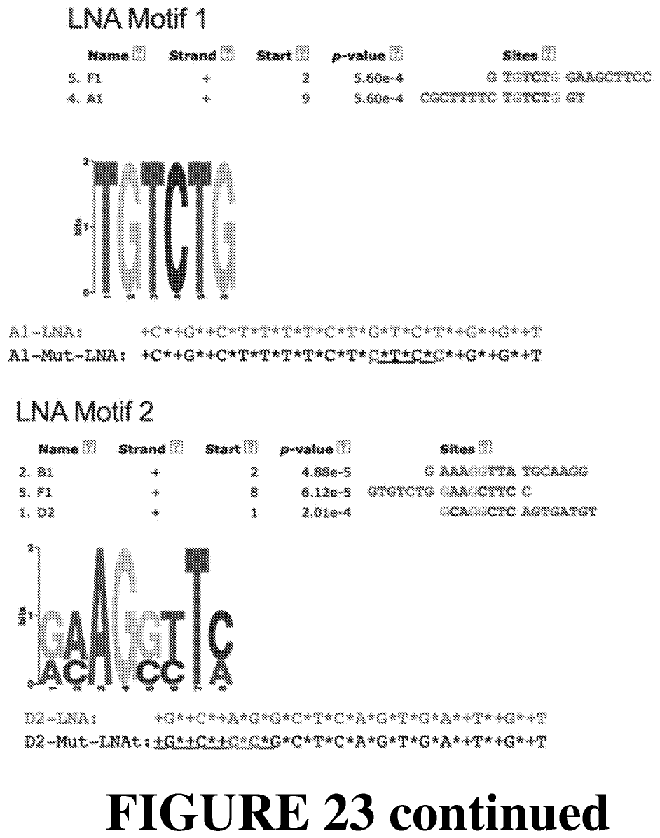

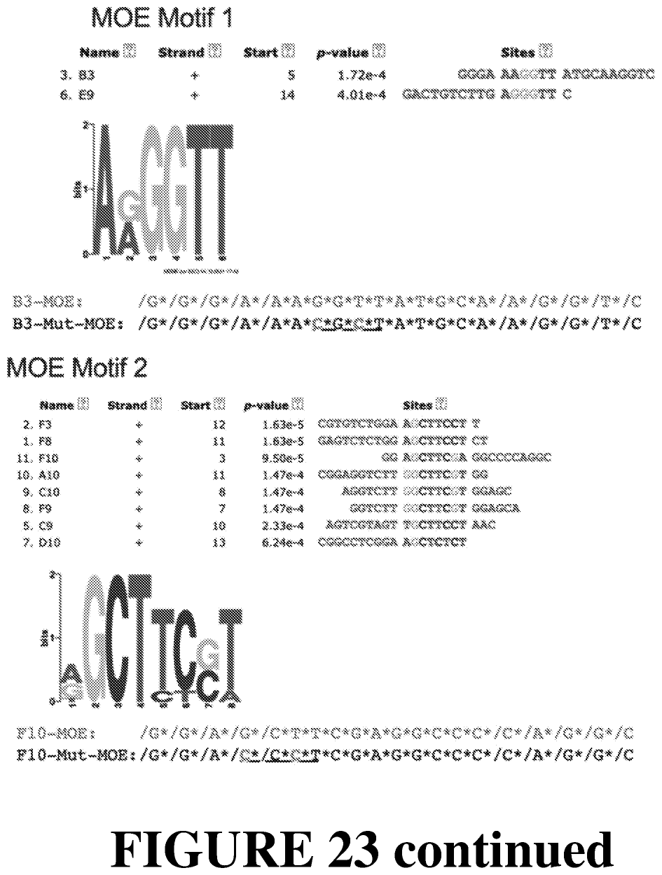

Motif discovery analyses was then performed with the MEME sequence analysis tool on the best cGAS inhibitors confirmed in human cells. For LNA ASOs, the present inventors first focused on a G*T*C*T (SEQ ID NO: 62) motif conserved between A1 and F1, which were both significant inhibitors of cGAS in THP-1 cells. The present inventors mutated this 1^{st} motif into a C*T*C*C (SEQ ID NO: 64) motif in A1-Mut (FIG. 23). In addition, the present inventors were interested to confirm the sequence-specific and species-specific effect of D2 (which inhibited in human cells but potentiated in mouse cells). Alignment of the 5 best ASOs in THP-1 assays identified a 2^{nd} motif conserved between B1, F1 and D2 which was mutated in D2 at position 3 and 4—the most conserved bases. This resulted in D2-Mut (FIG. 23).

Similarly, the present inventors looked for enriched motifs in the 11 MOE ASOs which strongly inhibited cGAS in THP-1 cells. The first MOE motif investigated was very conserved between B3 (the most potent ASO in THP-1 also inhibiting in mouse LL171 cells) and E9. Importantly, this G*G*T*T (SEQ ID NO: 72) motif was very similar to the G*G*T*A (SEQ ID NO: 350) motif from the 2′OMe C2-Mut1 ASO. This motif was mutated in B3 to obtain C*G*C*T (SEQ ID NO: 351) in B3-Mut. The second MOE motif selected was highly enriched in 8/11 ASOs. The conserved G*C*T*T (SEQ ID NO: 80) was mutated into C*C*C*T (SEQ ID NO: 352) within F10 (resulting in F10-Mut) (FIG. 23).

These ASOs and their mutants were tested in THP-1 cells at a single dose (using 200 nM for MOE ASOs, and 300 nM for the LNA, which were less potent), and also tested them in LL171 cells.

In THP-1 cells, the mutants of B3-MOE, F10-MOE and D2-LNA all lost the capacity to inhibit cGAS signalling, establishing the importance of these specific motifs in cGAS inhibition. Surprisingly, the 2-base modifications of A1-LNA rather significantly increased cGAS inhibition in human cells (similar to what the present inventors obtained when they discovered C2-mut1 was more potent than the parent 2′OMe C2 ASO (Valentin et al., 2021)) (FIG. 23). This suggests that bases substitutions rather improved cGAS inhibition for this motif.

In mouse LL171 cells, the mutations of B3-MOE and F10-MOE also significantly altered cGAS inhibition (FIG. 23). Contrarily to human cells where it was a more potent inhibitor of cGAS, the mutant of A 1-LNA lost inhibitory activity in mouse cells and rather potentiated signalling. In addition, the mutation of D2-LNA significantly thwarted its potentiating effect in mouse cells (FIG. 23).

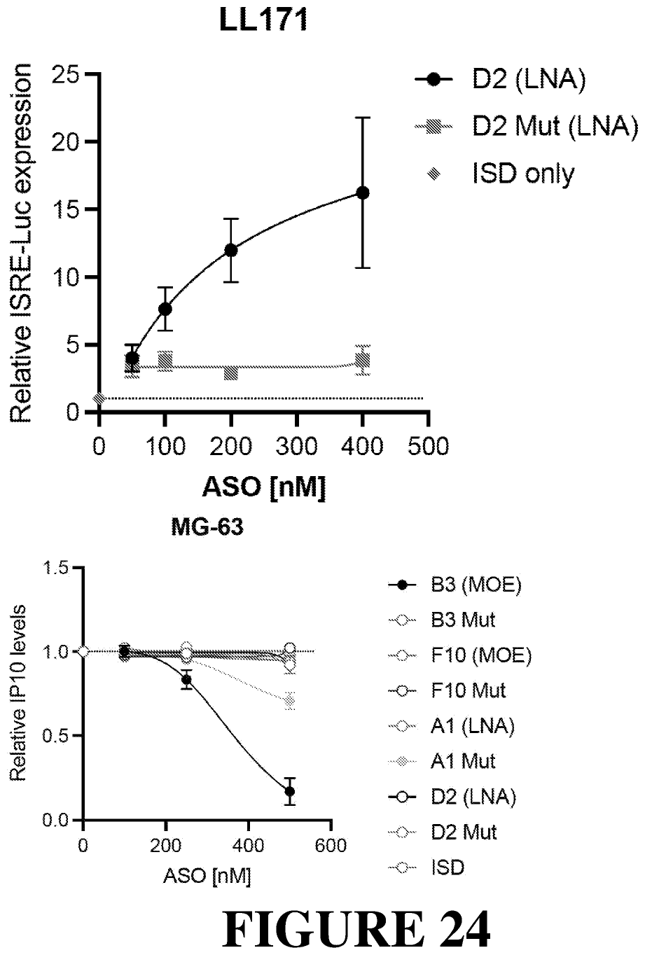

These observation for the LNA ASOs on mouse LL171 cells were confirmed in dose-response studies—where increasing amount of D2-LNA increased potentiation of ISD sensing (FIG. 24).

The present inventors also tested the inhibitory effects of their ASOs and their mutants in human MG-63 osteosarcoma cells—which are responsive to cGAS ligands (Valentin et al., 2021)—to broaden their findings beyond the case of human monocytic cells. Interestingly, in MG-63 cells, the ASOs were not as potent inhibitors of cGAS and only B3 and A1-Mut significantly reduced IP-10 levels at the doses tested. Critically, B3-Mut and A1 did not inhibit IP-10 production, confirming the sequence-specific effects of MOE and LNA ASOs in these cells (FIG. 24).

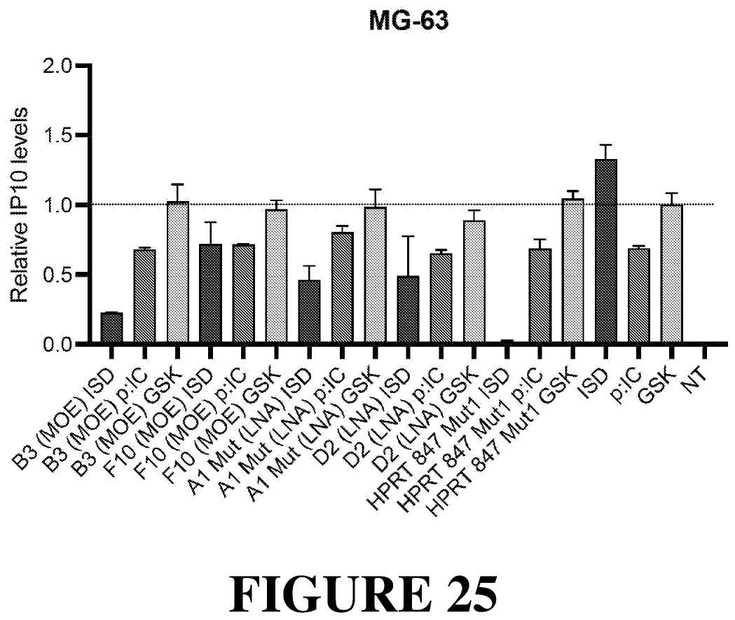

Next, specificity of inhibition was assessed in MG-63 cells using a higher dose of ASOs (1 mM) for F10, A1-Mut and D2. In one preliminary experiment, all the ASOs only reduced IP-10 driven by ISD, and not that driven by direct stimulation of STING with the GSK synthetic agonist, or by TLR3 engagement with polyLC (FIG. 25).

Finally, the present inventors wanted to test whether the observations made in mouse LL171 cells could also be replicated in mouse macrophages. The best ASOs and associated mutants were tested in immortalised mouse bone marrow derived macrophages (iBMDMs), stimulated with ISD, looking at IP-10 production as a read-out. While B3 and F10 significantly reduced ISD-induced IP-10, none of the other ASOs were inhibitory in this context. Surprisingly, A1-Mut and D2 did not significantly potentiate IP-10 production, in opposition to what was seen in fibroblast LL171 cells. While cGAS sensing may be differentially regulated between the macrophages and LL171 cells, it is also possible that kinetics of potentiation may be different and mask early potentiation using an overnight time point.

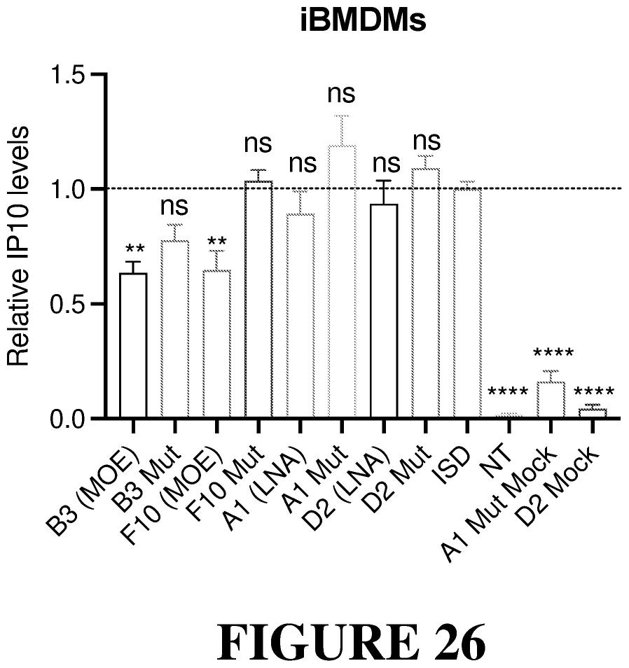

Nonetheless, the A1-Mut induced low level IP-10 when transfected alone in the absence of ISD. Whether this is due to cGAS activation remains to be confirmed, but this aligns with the lack of inhibitory activity of this sequence on ISD sensing (FIG. 26).

Collectively these results establish the motif-specific inhibitory effects of 2MOE and LNA ASOs on human and mouse cGAS sensing of DNA. Critically, the LNA motifs modulating cGAS activity in mouse fibroblasts had opposite effects in human cells-suggesting that at least for LNA modified ASOs there are critical inter-species differences to consider regarding their modulation of cGAS activity.

### Example 15: Analyses of 2′MOE and LNA modified ASOs potency compared to C2-Mut1

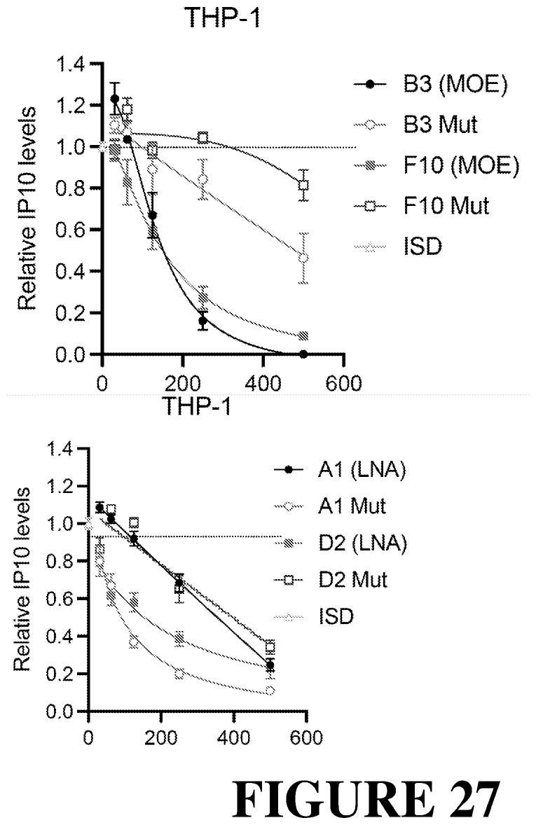

The present inventors have previously reported that the IC50 of C2-Mutl was ˜56 nM in THP-1 cells. The present inventors next assessed the inhibitory effect of the LNA and MOE ASOs and their sequence mutants, using dose-responses studies in THP-1 cells. First, for 2MOE ASOs, the present inventors determined that the IC50 of B3 and F10 were 133 and 147 nM, respectively (FIG. 27). Motif mutation of B3 and F10 strongly impacted their inhibitory activities, with F10-Mut performing the worst.

Second, the present inventors independently determined that the IC50 of A1-Mut and D2 were 75 and 212 nM, respectively. Motif mutants were also strongly impacted (FIG. 27). Collectively, these analyses indicated that 2′MOE B3 and LNA A1-Mut were the most potent inhibitors of cGAS in THP-1 cells (aligning with what had found previously in MG-63 cells).

Whether A1-Mut is more potent than B3 however remains to be confirmed in head-to-head comparisons, in the same experiments. Nonetheless the present inventors note that B3 was a much more potent cGAS inhibitor in MG-63 cells suggesting that it is a more robust inhibitor than A1-Mut overall between cells lines (FIGS. 27 and 24).

Interestingly, there is a discrepancy of activity between THP-1 and MG-63 cells regarding the activity of the F10 MOE ASO (FIGS. 27 and 24). While very potent in THP-1, this ASO failed to inhibit cGAS under 500 nM in MG-63 cells (although it was inhibitory when used at 1 mM—FIG. 25).

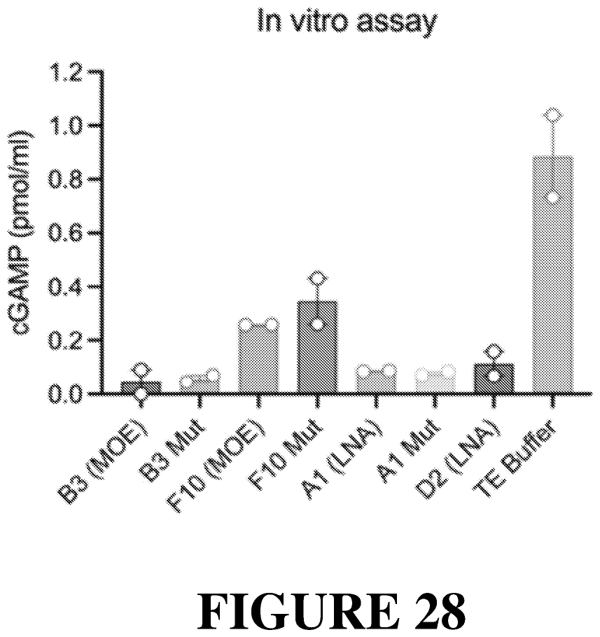

Finally, a preliminary experiment was performed to assess the capacity of the MOE and LNA ASOs to inhibit human cGAS enzymatic activity in vitro. In this system, recombinant cGAS is incubated with ISD70 in the presence or absence of the ASOs and cGAMP formation is subsequently assessed using a specific cGAMP ELISA (Valentin et al., 2021). The present inventors tested the ASOs at 2 μM as per their previous studies (Valentin et al., 2021). Surprisingly, while all the ASOs and their respective mutants strongly inhibited cGAMP production at this dose, F10 and F10-Mutant had a much weaker inhibitory activity (FIG. 28). These observations confirm that these ASOs directly compete with ISD for cGAS binding, thereby decreasing cGAMP activation. Nonetheless, at the dose used the mutants did not display significant differences to their parental sequences—which may be seen at different ASO doses. Critically, the observation that F10 and its mutant were less potent inhibitors indicates that their modality of action in vitro and in vivo differs. This aligns with the different activity of this ASO between MG-63 and THP-1 and argues for a different mechanism of action from the other ASOs which strongly impact cGAS activation in vitro.

Without being bound by any theory, it is proposed that F10 may be cleaved by a cellular nuclease, prior to being able to optimally engage cGAS and inhibit it (explaining the lower inhibitory activity in the in vitro assay). Alternatively, F10 may only bind cGAS in complex with a co-partner protein such as G3BP1 (Liu et al., 2019)-which is not possible in this vitro setup. Discrepancies seen between MG-63 and THP-1 would therefore rely on different expression of such a nuclease or co-partner protein. Further studies will be required to investigate how F10 impacts cGAS sensing of DNA.

### Example 16: Analyses of 2′MOE and LNA Modified ASOs Potency for TLR9 Inhibition

Previous Examples have shown that 2′OMe ASOs could also inhibit human TLR9, although this effect was moderate with only 8/80 ASOs significantly inhibiting TLR9 by more than 50% at 500 nM. To define whether TLR9 inhibition could be promoted by other ASO chemical modifications, the present inventors screened 91 LNA and 76 2′MOE modified ASOs for reduction of NF-κB-Luciferase upon ODN2006 treatment of HEK-TLR9 cells.

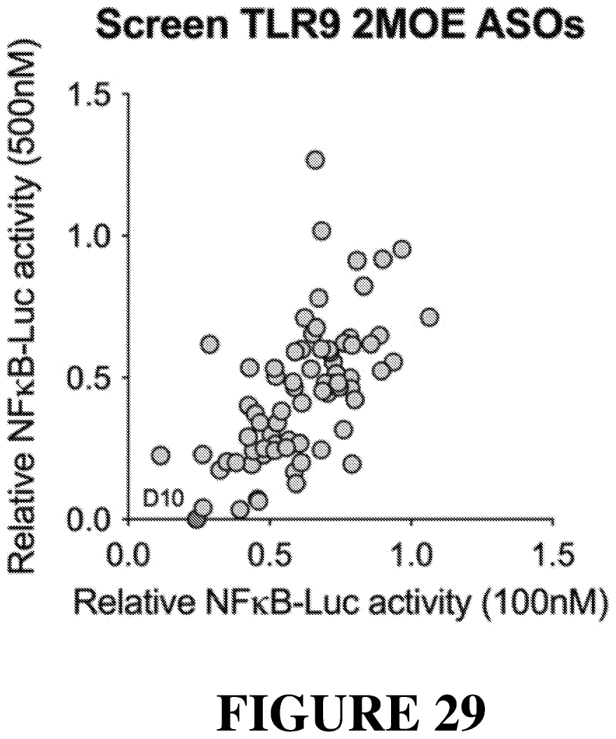

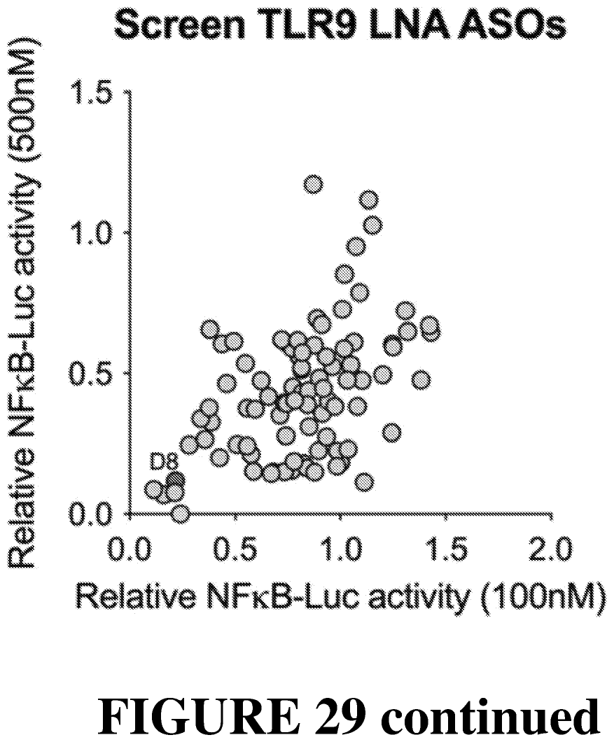

The overall repression appeared similar with 2MOE and LNA ASOs, and significantly greater than that seen with 2′OMe ASOs. As such, 57/91 (i.e. 62.6%) LNA ASOs repressed TLR9 signal by more than 50% at 500 nM, versus 46/76 (i.e. 60.5%) for 2MOE ASOs (FIG. 29).

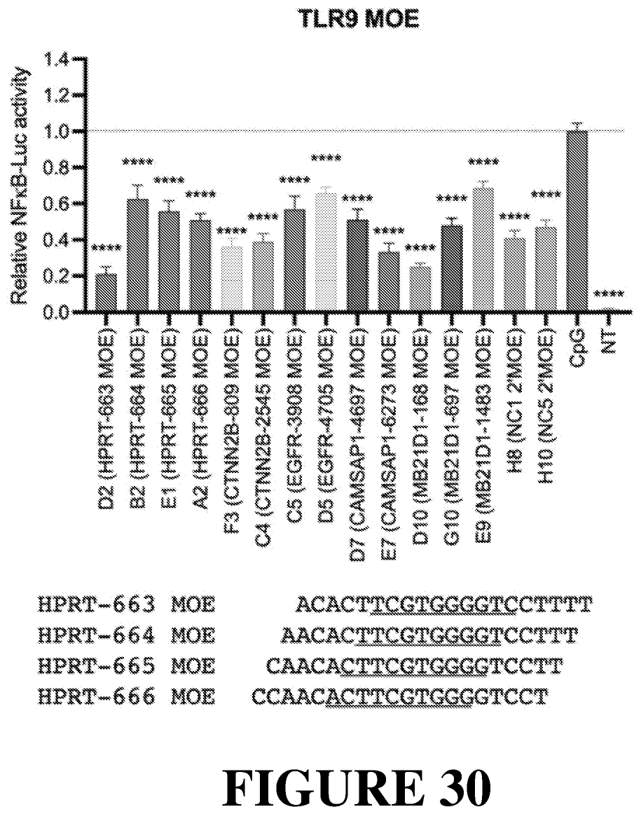

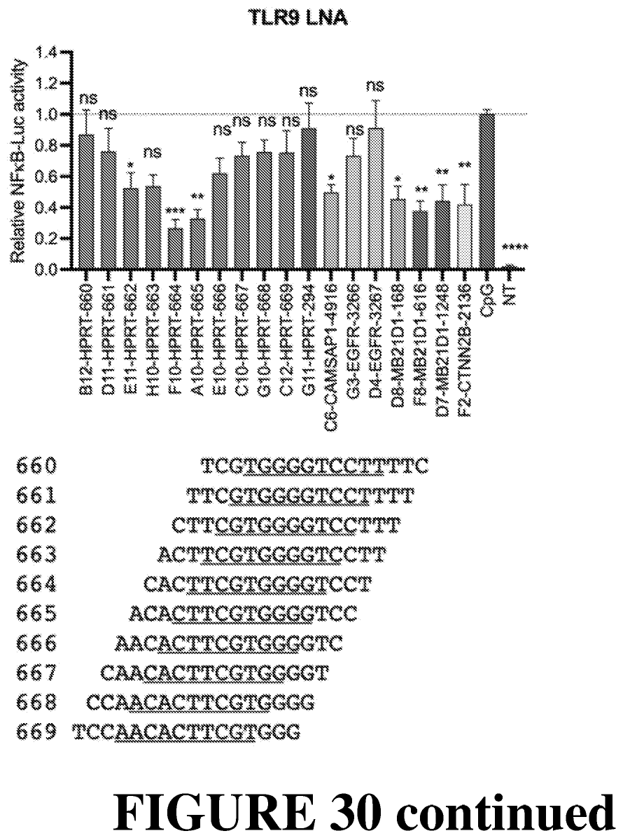

The present inventors next looked to validate the top targets from each screen. For 2MOE, it is noted that one of the best inhibitors, HPRT-663 (D2) was closely related to 3 other sequences with base pair increments which were also included in the validations. Collectively, all the ASOs tested here at 100 nM significantly inhibited TLR9, with D2 and D10 (ASO2) being the most potent (FIG. 30). Critically, HPRT-663 was a stronger inhibitor than 664/665 and 666 ASOs indicating an important contribution from its 5′ or 3′ ends.

For LNA ASOs, the effect on TLR9 was also clearly sequence-dependent with a strong impact of incremental bases in the HPRT series (660-669). As such, while 660 was not inhibitory, there was a progressive increase in inhibition peaking with 664 and 665 LNA ASOs, and decreasing again with 666 and further ASOs in the series. This suggests that the 5′ and 3′ end termini are likely at play here. D8 (ASO2) was also significantly inhibiting in LNA chemistry.

It should be noted that the LNA chemistry relies on 3-mer wings in the gapmer, instead of 5-mers for 2′OMe and 2′MOE; hence the sequences differ slightly for LNA and the two other chemistries. Interestingly, AS0663 in 2MOE is quite inhibitory and AS0665 in LNA is also inhibitory. When looking at the 5′end of these sequences, the present inventors note that AS0663 MOE is composed of a 5′end “ACA” motif, which is also seen in AS0665 LNA. Similarly, the 5′end of AS0662 2′OMe is “CAC” which is also seen in AS0664 LNA and is also inhibitory. These specific 5′end “ACA” and “CAC” motifs may therefore be related to the TLR9 inhibitory effect of the ASOs, independent of the chemistries used.

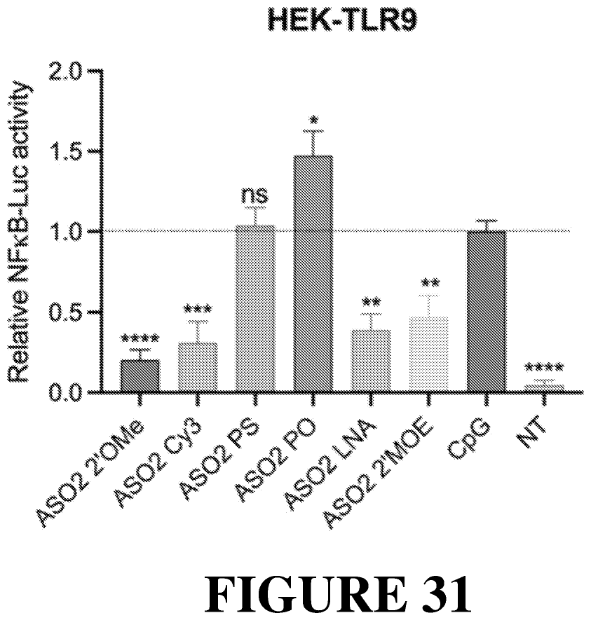

While the ends of the ASOs are impacting TLR9 sensing with the 3 chemistries, it is noted that the ASOs targeting 168-MB21D1, which is referred to as ASO2 (Valentin et al., 2021), were consistently a very strong inhibitor of TLR9—rather suggesting a key role for the central 16 nt in common between all chemistries. The present inventors therefore tested a panel of 2′OMe ASO2 variants including: ASO2 on a phosphodiester backbone (PO), lacking the 2′OMe moieties (PS), or containing a 3′-end Cy3 moiety, compared to ASO2 in 2′OMe, LNA and MOE chemistries (as per FIG. 1G in (Alharbi et al., 2020)). These experiments confirmed the need for 5′ and 3′-end modifications for TLR9 inhibition since the PS ASO2 entirely lacked its inhibitory activity on TLR9. Interestingly, the PO variant rather significantly potentiated TLR9 sensing—indicating that the sequence of ASO2 is likely directly facilitating interaction with TLR9 (FIG. 31). This induction with the PO ASO2 also indicated that its lack of inhibitory activity was not related to its lack of uptake by the HEKs. Nonetheless, as suggested from their independent experiments, ASO2 significantly inhibited TLR9 when designed with the 2′OMe, LNA and MOE backbone with comparable efficacy (FIG. 31).

### Example 17: Inhibition of RNA Sensing by TLR7

Having shown C2-Mutl and Mutl-dC inhibited TLR7 engagement with Guanosine, the present inventors also tested its effect on an immunostimulatory short single stranded RNA. For this experiment, a synthetic ssRNA (i.e., B-406-AS ssRNA (UAAUUGGCGUCUGGCCUUCUU, SEQ ID NO: 345)) was utilised, which the inventors have previously found to activate TLR7 (Gantier et al., 2010). This ssRNA was transfected with DOTAP (Roche) and pure DMEM in biological triplicate, as previously described (Gantier et al., 2010), to a final concentration of 250 nM. The ratio of DOTAP to RNA (at 80 uM) was 3.52 ug/ul of ssRNA.

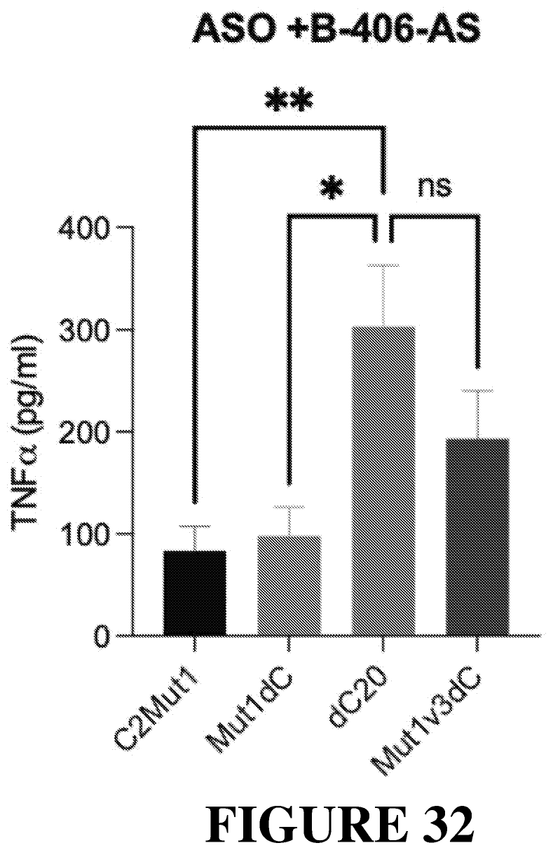

As shown in FIG. 32, pre-treatment of the primary BMDMs with C2-Mutl and Mutl-dC, but not Mutl-v3-dC, significantly reduced TNFα production induced by ssRNA sensing compared to the dC20 control co-treatment, further confirming the sequence specific inhibition by the oligonucleotides described herein (and in particular the 5′-GGUAU-3′ (SEQ ID NO: 56) motif) of TLR7 signalling driven by RNA.

### Example 18: TLR8 Potentiation by 2′O-Methyl ASOs is Dependent on 3-Base Motifs

Previous work has shown the sequence specific potentiation of TLR8 with the example of ASO2 (2′OMe), but not its LNA variant ASO2-LNA, which lacks the terminal 5′mUmC motif (see below). When adding back this 5′mUmC motif to AS02-LNA (giving AS02-LNA-Mutl) there was no impact on TLR8 potentiation, suggesting that the +C+G (where “+” denotes an LNA base) bases somehow antagonised the effect of the 5′ UCCGG motif, otherwise found to directly potentiate TLR8 (as seen in ASO11-Mut2, see below).

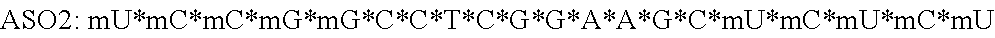

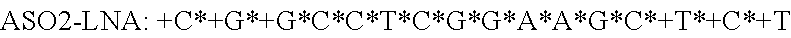

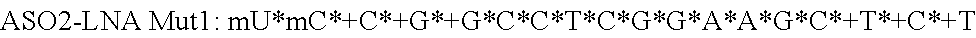

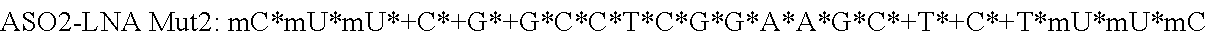

2′Ome AS0660 is also a strong potentiator of TLR8 sensing. Such potentiation was directly dependent on a 5′ mCmUmU[mCmG] motif, where “m” denotes a 2′OMethyl base, but was not seen in the context of a 5′ mCmUmU[+C+G], in ASO2-LNA Mut2 (see above). This suggested again that the +C+G motif in the ASO2-LNA Mut2 was somehow antagonising the effect of the CUU (SEQ ID NO: 153) motif [PCT2021/050469].

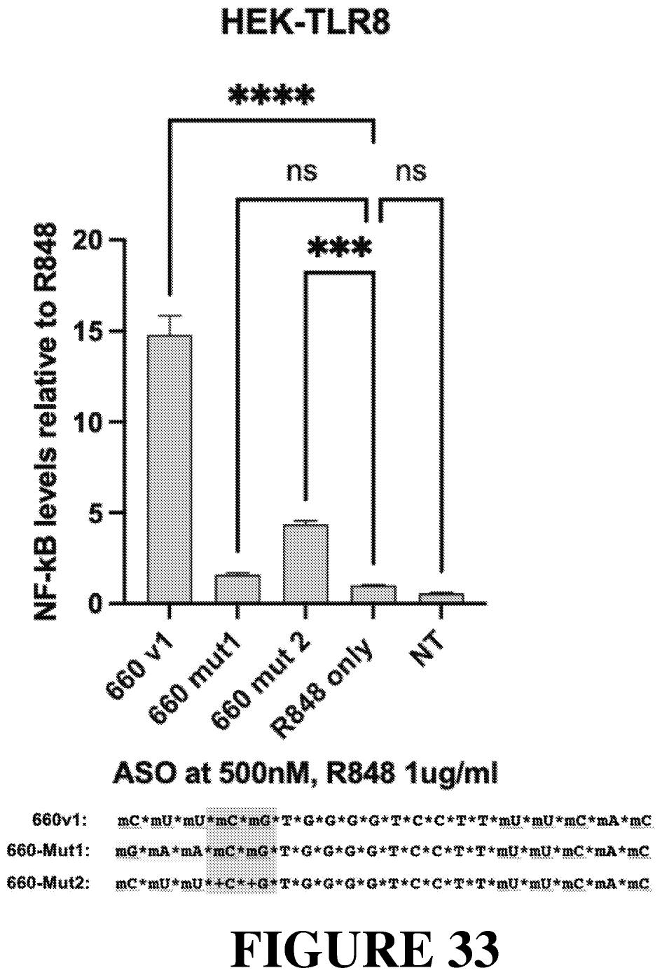

To extend these results, here the present inventors synthesised ASO 660-Mut2 with the 5′ mCmUmU[mCmG] motif changed into 5′ mCmUmU[+C+G] motif, but otherwise keeping the entire sequence unaltered (FIG. 33). These experiments demonstrated that changing these two bases strongly reduced TLR8 potentiation of R848 sensing for 660-Mut2 (FIG. 33).

Collectively these results directly supported a key role for the “mCmG” residues of 660 and ASO2 in TLR8 potentiation—which was strongly decreased when these bases were replaced into LNA bases.

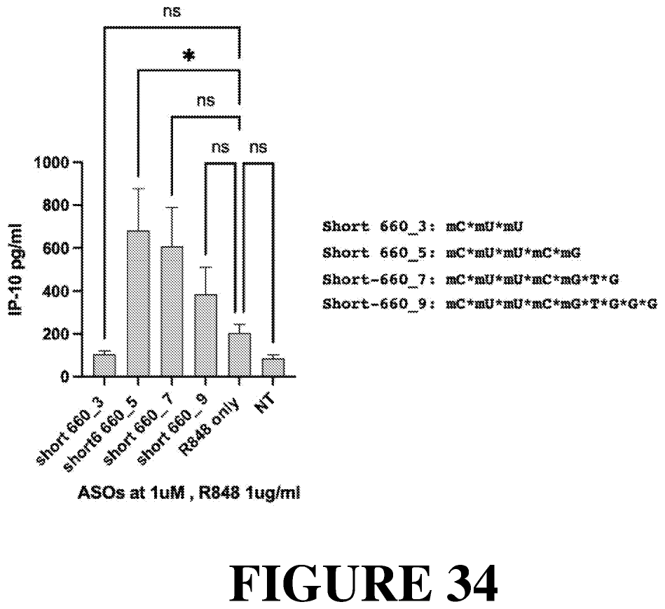

Speculating that these two bases would condition the processing of ASO 660 by endo/exonucleases to release 5′ end products, the present inventors next synthesised a series of short 2′OMe ASOs of different lengths reproducing the 5′end of ASO 660 (referred to as Short-660 oligos). Overnight incubation of these short oligonucleotides prior to R848 stimulation demonstrated a length-dependent induction of IP-10 production by THP-1 cells, which was strongest with the last five 5′end bases (noting that the terminal CUU alone did not potentiate TLR8-FIG. 34).

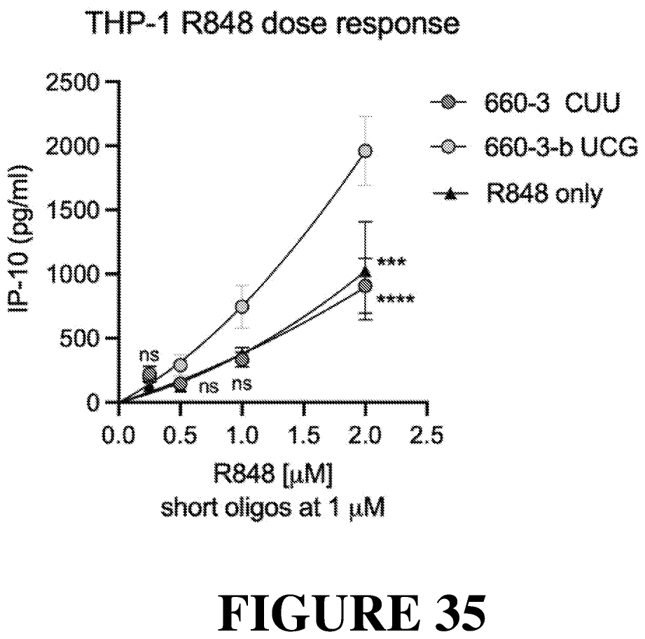

The capacity to potentiate TLR8 with 5 bases lead the present inventors to speculate that, similar to TLR7, the effector motif on TLR8 might actually be shorter. Since mCmUmU (660-3) did not work, the present inventors also tested whether another 3 base-long oligo encompassing the important “mCmG” bases of ASO 660 could modulate TLR8 function (mUmCmG, referred to as 660-3b). Surprisingly, 660-3b significantly potentiated TLR8 sensing compared to 660-3 and R848 alone, with increasing activity with increasing levels of R848 (FIG. 35).

Based on these previous findings, the present inventors next performed a screen of the 64 possible combinations of 3 bases on R848 sensing in HEK-TLR8 cells. The present inventors tested 3-mers made of 2′OMe bases on a phosphorothioate (PS) backbone, using 5 μM of naked oligos (i.e. non transfected) and 600 ng/ml of the TLR8 selective agonist Motolimod, in biological triplicate.

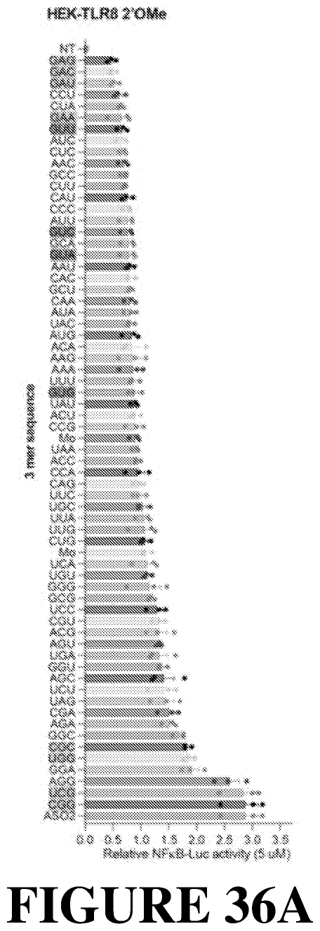

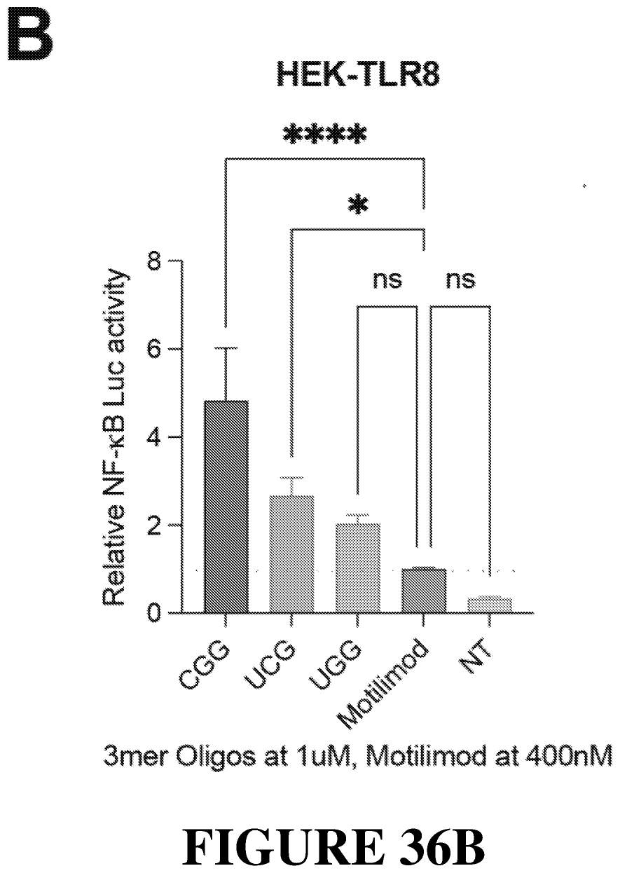

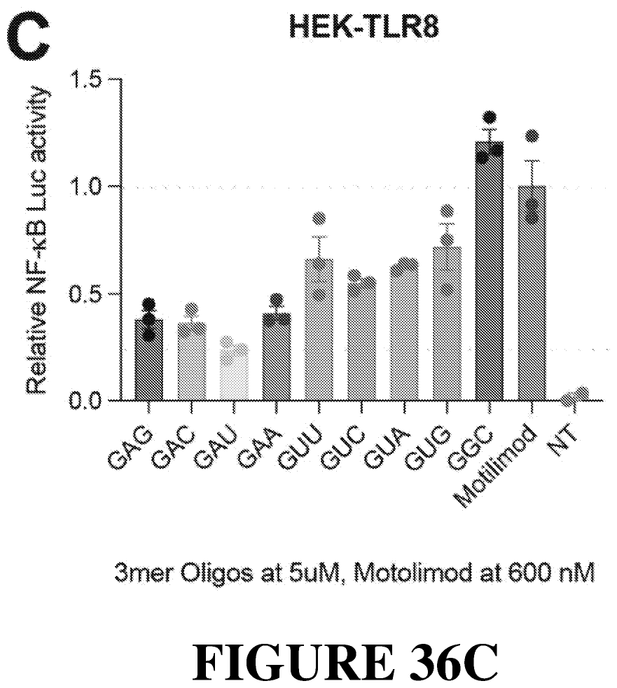

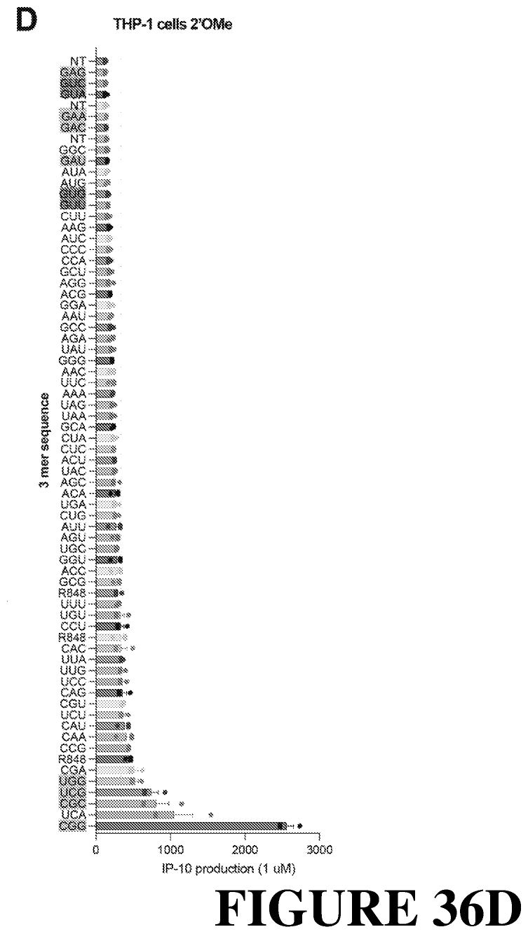

As shown in FIG. 36, only a few 3-mers potentiated the sensing of R848, with “CGG” (SEQ ID NO: 383) being the most potent sequence, followed by “UCG” (i.e. 660-3b), “UGG”, and “CGC” (SEQ ID NOs 451, 455, 375) in decreasing order of potency (also noting the potentiating effect of “AGG” and “GGA” (SEQ ID NOs: 381 and 370) in this screen). The first 3 potentiators (“CGG”, “UCG”, “UGG”; SEQ ID Nos: 383, 451, 455) were independently validated in repeat experiments, confirming that CGC and UCG significantly potentiated TLR8 sensing of Motolimod. Critically, the 4 sequences listed above were also found to be among the top potentiator of IP-10 production in the repeat screen of our 64 3-mers in THP-1 cells stimulated with R848-confirming that the observation is robustly seen in different TLR8 cell models (FIG. 36).

The very strong effect of “CGG” (SEQ ID NO 383) on R848/Motolimod sensing observed here is directly consistent with its presence in the 5′end of ASO2, and overlaps with the mCmG motif which, when mutated to +C+G with LNA bases lost a lot of activity.

Importantly, several 3-mer 2′OMe oligos appeared to inhibit Motolimod and R848 sensing (displaying IP-10 levels similar to those obtained without R848 in THP-1, and ˜50% decreased NF-κB luciferase activity in HEK-TLR8). In this inhibiting category, the “GAX” (SEQ ID NO: 591) molecules (i.e. “GAG”, “GAC”, “GAU” and “GAA”) were robustly the most potent in both HEK-TLR8 and THP-1 screens. “GUX” (SEQ ID NO 592) molecules (i.e. “GUC”, “GUU”, “GUA” and “GUG”) were also robustly inhibitory in both models, although not as potent as GAX molecules. These trends were independently confirmed in a repeat experiment in HEK-TLR8 cells, collectively demonstrating that a few 3-mers can inhibit TLR8 sensing (FIG. 36).

### Example 19: Inhibition of TLR7 by 2′O-Methyl ASOs

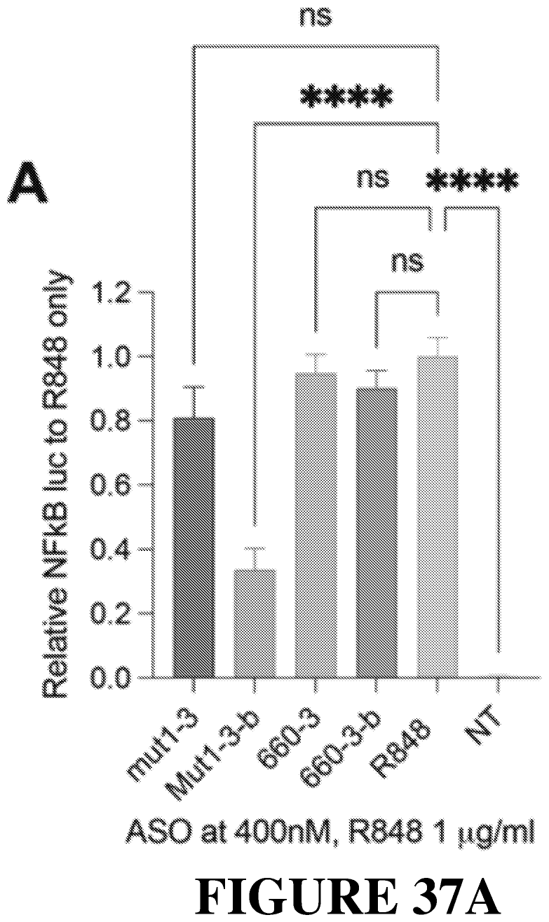

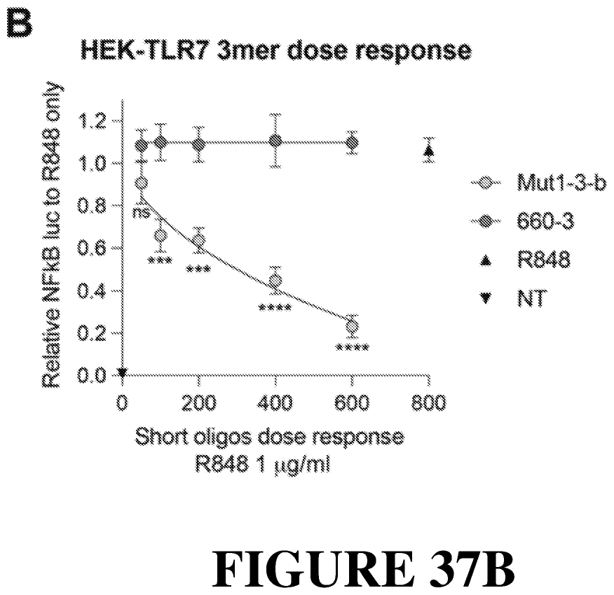

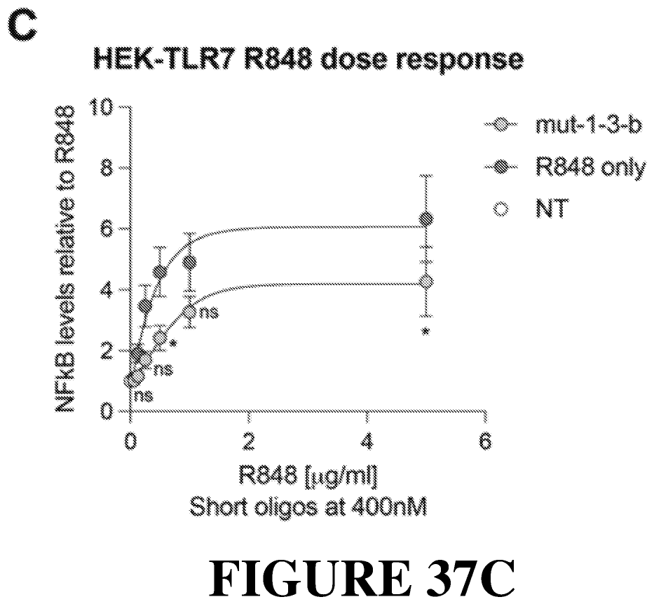

Previous Examples have shown that the Short Mutl “mGmGmUmAmU” 5-mer was sufficient to inhibit R848 sensing by TLR7 (FIG. 14). However, this inhibition was reliant upon a 6 h incubation of the oligo which suggested it may be processed further into shorter fragments. To address this possibility, the present inventors next investigated the effect of “mGmGmU” (shortMut1-3) and “mGmUmA” (shortMut1-3b) on TLR7 sensing, decreasing the incubation time of the oligos from 6 h to 30 min prior to R848 stimulation. The present inventors also included two other 3-mer sequences, 660-3 and 660-3b, as controls. These experiments confirmed that the effector motif in the Mut1 5-mer was in fact based on the “GAU” (SEQ ID NO: 440) 3-mer, which selectively inhibited TLR7 sensing (FIG. 37). Dose-response analyses demonstrated an IC50 of 300 nM with this 3-mer oligo, and inhibition was robustly seen with increasing amounts of R848 (FIG. 37).

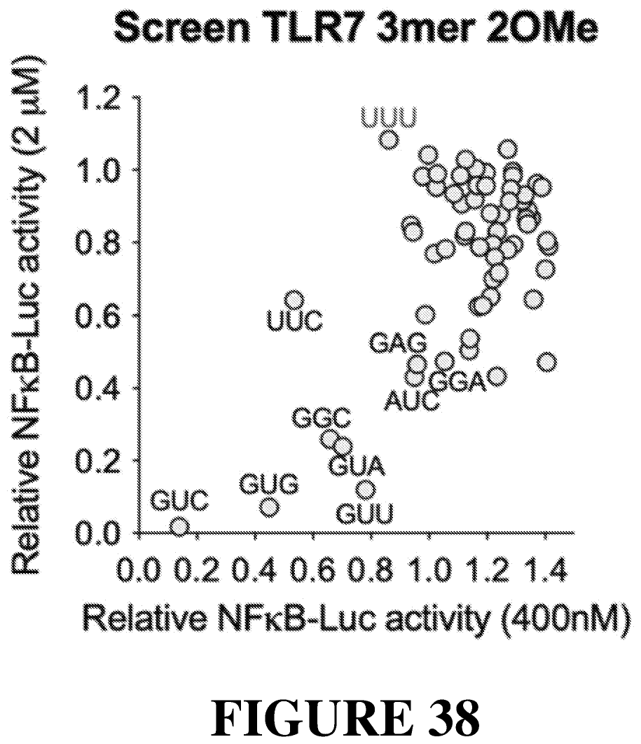

Based on these results, the present inventors performed a screen of the 64 possible combinations of 3 2′OMe bases on R848 sensing by TLR7. The present inventors performed two independent screens, at 400 nm and 2 μM, in biological triplicate (FIG. 38).

These screens demonstrated that the most potent 3-mer inhibiting TLR7 was “GUC”, followed by “GUG”, “GUA” and “GUU”, then “GGC”, “AUC”, “GAG” and “GGA” (SEQ ID NOs: 442, 443, 60,444,377, 431, 384, 370). The clear over-representation of “GUX” (SEQ ID NO:″592) suggested a specific activity of these bases on TLR7 binding to site 2. Importantly, the GUX motifs also inhibited TLR8, and the same was true for “GAG” (SEQ ID NO: 384). Given the proximity of structures between TLR7 and TLR8, these results strongly support a preferential binding of these specific motifs to site 2 of these receptors, leading to the blocking of both TLR7/8 activation by R848.

Since these ASOs contained 10 internal DNA bases, the present inventors posited that 3-mer DNA fragments may also impact TLR7 sensing and conducted a screen of the 64 possible 3-mer DNA bases on a PS backbone, at 2 μM in biological triplicate. These analyses demonstrated that the most potent 3-mer DNA inhibited TLR7 by ˜50%. Critically, the present inventors found that the two most potent DNA 3-mers inhibiting TLR7 were “TTT” and “TCT” (SEQ ID NOs: 425 and 424).

Collectively these analyses support a working model where the 20-mer gapmer ASOs used are rapidly degraded prior to be able to impact TLR7/8 sensing of R848 in the endolysosome. Since the present inventors can clearly replicate some of the inhibitory/potentiating activity with as few as 3-mer PS 2′OMe oligos, they believe that these degradation products are the effectors of the activity of longer 2′OMe ASOs on TLR7/8.

Since the 20-mers and the 3-mers do not need prolonged pre-treatment to act on TLR7/8 in HEK cells, but the 5-mer Mutl sequence did not inhibit without a 6 h incubation, the present inventors propose that endonuclease cleavage of the long 20-mer gapmers, probably through their middle DNA region, releases 2′OMe fragments that are further trimmed through exonuclease activity into 3-mers. Endo and exonucleases are probably coupled together which explains why 5-mers are less efficiently processed (length dependent uptake is unlikely the problem here since 3-mers are clearly efficiently taken up).

### Example 20: Length Dependent Inhibition of cGAS by 2′O-Methyl Modified ASOs

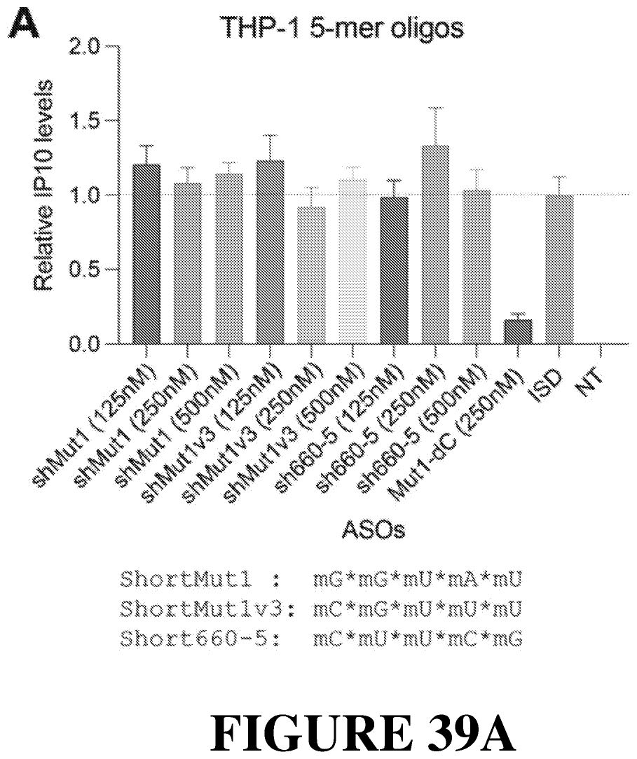

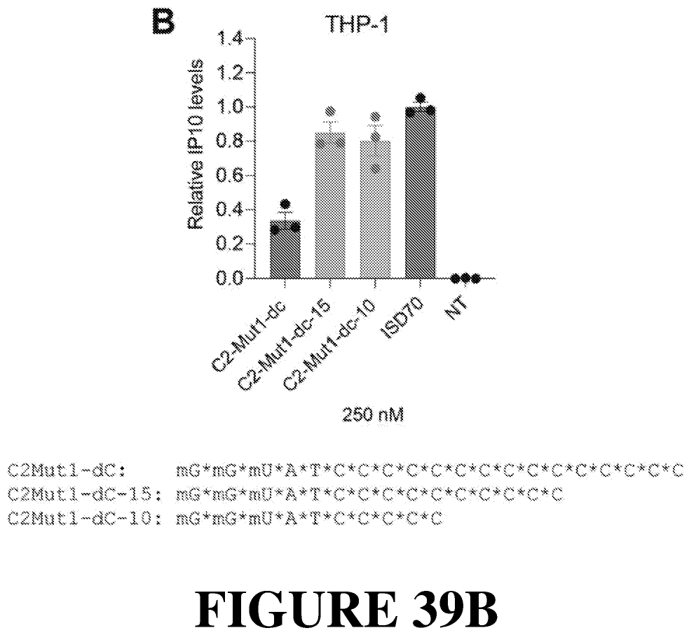

The previous Examples have shown that 20-mer long oligonucleotides containing a stretch of 17 dCs with as few as 3 2′OMe bases (Mut-1-dC-3) could inhibit cGAS sensing. Having shown that 5-mer oligos could inhibit TLR7 and potentiate TLR8, the present inventors investigated whether the 5-mer 2′OMe oligo with the Mut-1 motif (mG*mG*mU*mA*mU) could inhibit cGAS sensing. Independent of the dose used (125, 250 or 500 nM), none of the 5-mertested significantly inhibited cGAS sensing of transfected ISD70 in THP-1 cells (FIG. 39). To determine further the minimal length of an oligo required to inhibit cGAS, we generated two variants of C2-Mut1-dC, with 10 and 5 dC stretches at their 3′end (giving C2-Mutl-dC-15 and Mutl-dC-10). These shorter ASOs, albeit displaying the 5′ end mG*mG*mU*A*T motif of C2-Mutl-dC, did not inhibit cGAS (FIG. 39). Collectively, these results suggest that unlike that of TLR7/8, inhibition of cGAS sensing by 2′OMe ASOs is constrained by the length of the oligos used. In accord with this, LNA ASOs, which are only 16 bases, were overall less potent inhibitors of cGAS sensing compared to 2′OMe and 2′MOE 20-mer ASOs (FIG. 20).

These results indicate that unlike that of TLR7/8, inhibition of cGAS sensing is not mediated by degradation products of the 20-mer ASOs such as C2-Mutl and is rather conditioned by a minimum length>15-mer.

### Example 21: TLR9 Inhibition

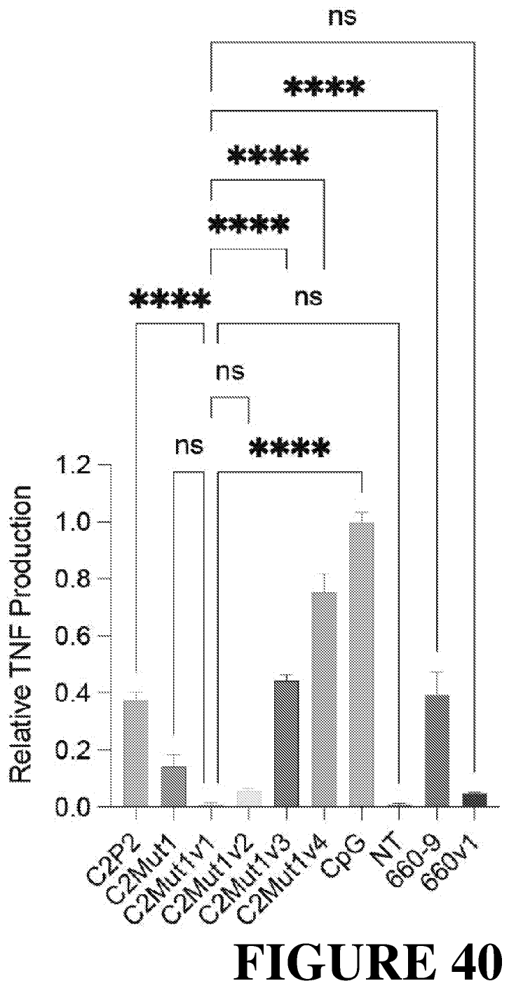

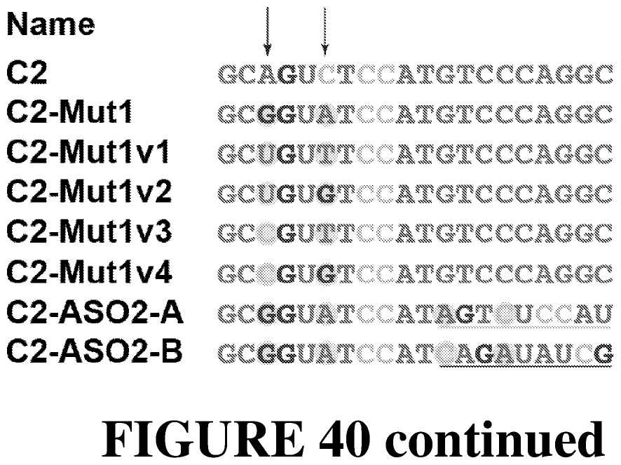

The previous Examples have shown that 2′OMe, 2′MOE and 2′LNA PS ASOs could inhibit human TLR9 in a sequence-specific manner. Interestingly, the present inventors have also found that C2-Mutl was a very potent inhibitor of mouse TLR9 (while being much less potent on human TLR9). To define whether TLR9 inhibition could be narrowed down to a specific motif, which has not been shown to that stage, the inventors tested their panel of C2-Mutl variants on mouse TLR9 inhibition (FIG. 40). These analyses demonstrated that single base substitutions withing C2-Mut1 had a drastic effect on the inhibition of mouse TLR9 sensing, with the example of C2-Mut1vl and C2-Mut1v3 which only differ by one base (from UGUTT (SEQ ID NO: 596) to CGUTT (SEQ ID NO 597)), and strongly impairs inhibition. Critically, the present inventors also found that ASO 660v1, which inhibited human and mouse TLR9, retained inhibitory activity when cropped to the 9 bases from its 5′ end (FIG. 40).

Albeit clear differences existed between human and mouse TLR9 sensing, these findings demonstrated that mouse TLR9 inhibition was also directly dependent selective 2′OMe motifs—suggesting the same for human TLR9 (based on our prior analyses of ASO2)—and that molecules shorter than 20-mer could be inhibitory.

Previous studies have shown that TLR9 sensing of selected CpG ASO motifs was dependent on cleavage of longer ASOs by DNase II. Having demonstrated that 3-mer 2′OMe oligonucleotides could modulate TLR7/8, the present inventors hypothesised that 3-mer degradation products from longer ASOs may also control TLR9 sensing and tested their 2′OMe panel of 64 3-mers on human TLR9 sensing.

The 2′OMe panel revealed that TLR9 sensing was marginally inhibited (less than 50%) by select 2′OMe 3-mers: “ACC”, “CGC”, “GAU”, “GGG”, and the most potent, “UCG” and “ACG” (SEQ ID Nos: 403, 375, 440, 385, 451, 411) (FIG. 41). Nonetheless the inhibitory effect of these few sequences was mild compared to that seen on TLR7 at this high dose of 3-mer, although this could be due to a saturation of TLR9 sensing with the agonist. Interestingly, a few of these inhibitors also modulated the function of TLR8 with GAU (SEQ ID NO: 440) (TLR8 inhibition), and UCG/CGC (SEQ ID NO: 451/375) (TLR8 potentiation).

Collectively, these findings suggest that TLR9 can be inhibited by 2′OMe PS oligos of at least 9 bases, and that select 2′OMe 3-mer oligos can also have an effect on sensing.

### Example 22: TLR3 Inhibition

Having shown that 3-mer PS oligonucleotides could control TLR7/8 and 9 sensing, the present inventors speculated that selected motifs may also control TLR3 sensing—the concept being that endosomal sensing by TLR3/7/8/9 could be broadly impacting by short degradation products of longer PS oligonucleotides.

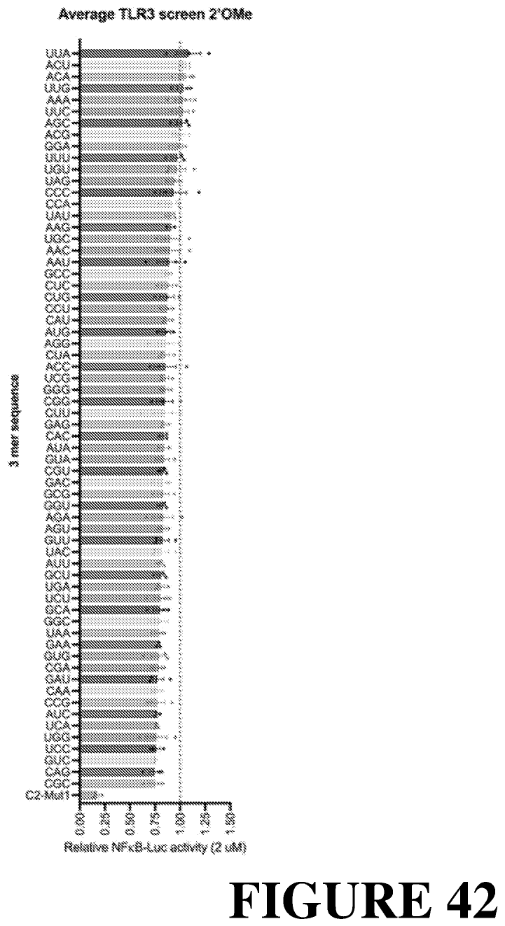

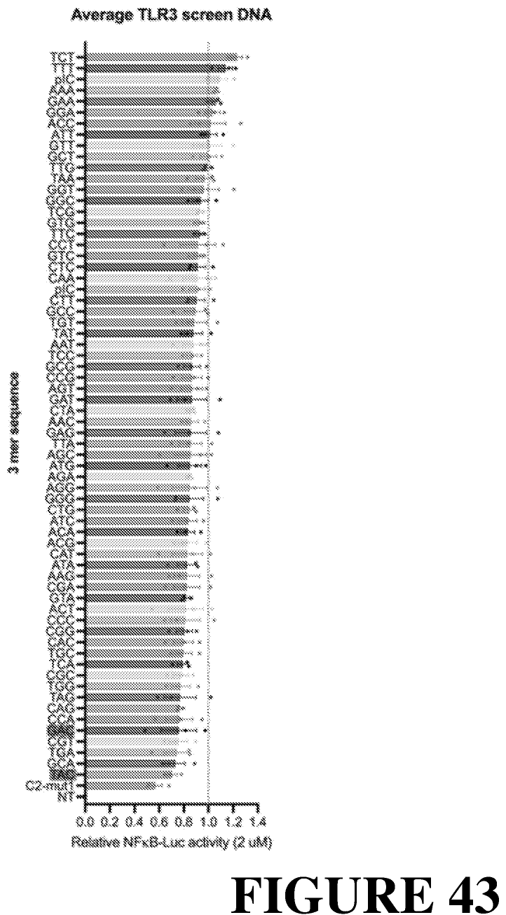

The present inventors therefore tested their 2′OMe and DNA panels of 64 3-mers on human TLR3 sensing (FIG. 42 and FIG. 43). For both panels, the inhibitory effect was between ˜25-30%, with the strongest DNA 3-mer “TAC” (SEQ ID NO: 378) and the strongest 2′OMe 3-mer “CGC” (SEQ ID NO: 375). The present inventors further observed that “GCA”, “UGA”, “CAG”, “UGG”, “CGC” and “UCA” (where U can be a T) (SEQ ID Nos: 399, 453, 382, 455, 375, 449) were inhibiting>20% with both DNA and 2′OMe.

These experiments collectively demonstrate that the 3-mers only have an inhibitory effect on RNA sensing by TLR3, when used at a high dose (2 μM).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters for part of the prior art base or were coon general knowledge in the field relevant to the present invention as it existed before the priority date of each claim of this application.

### REFERENCES

Ablasser et al. (2013) Nature. 503:530-534.
Alharbi et al. (2020) Nucleic Acids Res. 48:7052-7065.
An et al. (2018) Arthritis Rheumatol. 70:1807-1819.
Arnett et al. (1988) Arthritis & Rheumatis. 31:315-324.
Bailey and Elkan (1994) Proc Int Conf Intel Syst Mol Biol. 2:28-36.
Barrat et al. (2005) J Exp Med. 202:1131-1139.
Bayik et al. (2016) Pharacol Res. 105:216-225.
Beignon et al. (2005) J Clin Invest. 115:3265-3275.
Bode et al. (2021) Eur J Immunol. 51:1686-97.
Byrne et al. (2020) BMJ. 368:579.
Capisi (2005) Cell. 120:513-520.
Dai et al. (2019) Cell. 176:1447-1460 e1414.
Devarkar et al. (2016) Proc Natl Acad Sci USA. 113:596-601.
Dou et al. (2017) Nature. 550:402-406.
Esposito et al. (2018) Genes 9:529.
Ferrand and Gantier (2016) methods Mol Biol. 1390:79-90.
Ferrand et al. (2018) Frontiers in Cellular and Infection icrobiology. 8.
Frazier (2015) Toxicol Pathol. 43:78-89.
Gantier et al. (2010) Mol Ther. 18:785-95.
Gluck et al. (2017) Nat Cell Biol. 19:1061-1070.
Gray et al. (2015) The Journal of Immunology. 195:1939-1943.
Greulich et al. (2019) Cell. 179: 1264-75.
Gursel et al. (2003) J Immunol. 171:1393-1400.
Hall et al. (2017) PLoS One, 12:e0184843.
Heim et al. (2000) Nature. 408:740-745.
Judge et al. (2005) Nat Biotechnol. 23:457-462.
Judge et al. (2006) Mol Ther. 13:494-505.
Kaminski et al. (2013) J Immunol. 191:3876-3883.
Kranzusch et al. (2013) Cell Reports. 3:1362-1368.
Krieg et al. (1995) Nature 374:546-549.
Krieg et al. (1998) Proc Natl Acad Sci USA. 95:12631-12636.
Lama et al. (2019) Nat Commun. 10:2261.
Leech et al. (1999) Arthritis & Rheumatis. 42:1601-1608.
Liu et al. (2019) Nat Immunol. 20:18-28.
Mankan et al. (2014) The EMBO Journal. 33:2937-2946.
McWhirter and Jefferies (2020) Immunity. 53:78-97.
Nair et al. (2014) J. Am. Chem. Soc. 136:16958-16961.
Padilla-Salinas et al. (2020) J Org Chem. 85:1579-1600.
Pelka et al. (2014) J Immunol. 193:3257-3261
Pepin et al. (2017) mBio, 8.
Quin et al. (2016) Oncotarget. 7:49800-49818.
Raanjulu et al. (2018) Nature. 564:439-443.
Robbins et al. (2007) Mol Ther. 15:1663-1669.
Sarvestani et al. (2015) Nucleic Acids Res. 43:1177-1188.
Shibata et al. (2016) Int Immunol. 28:211-22.
Sioud et al. (2007) Bioche Biophys Res Coun. 361:122-126.
Steinhagen et al. (2018) Eur J Immunol. 48:605-611.
Stetson and Medzhitov (2006) Immunity. 24:93-103.
Toloue and Ford (2011) methods mol. Biol. 764:123-139.
Trieu et al. (2006) Crit Rev Immunol. 26:527-544.
Uhlen et al. (2017) Science. 357.
Unterholzner et al. (2010) Nat Immunol. 11:997-1004.
Valentin et al. (2021) Nucleic Acids Res. 49:6082-99.
Vincent et al. (2017) Nat Commun. 8:750.
Wang et al. (2018) Future Med Chem. 10:1301-1317.
Xia et al. (2016) Cell Rep. 14:282-297.
Xie et al. (2019) Proc Natl Acad Sci USA. 116:11946-11955.
Yang et al. (2017) PNAS 114:E4612-E4620.
Yin and Rogge (2019) Clin Transl Sci. 12:98-112.
Zhao et al. (2020) J Chem Inf Model. 60:3265-3276.

1. A method for selecting or designing an oligonucleotide which inhibits cGAS activity, the method comprising
i) scanning a polynucleotide, or complement thereof, for a region having a motif including a sequence selected from the group consisting of:
(1)
5′-[A/G]GU[A/C][U/C]C-3′;
(2)
5′-A[G/A][U/G]C[U/C]C-3′;
(3)
5′-A[G/A][U/G]C[U/C]C[U/C][C/A]U-3′;
(4)
5′-GGUAUA-3′;
(5)
5′-UGUUUC-3′;
(6)
5′-UGUGUC-3′;
(7)
5′-CGUUUC-3′;
(8)
5′-CGUGUC-3′;
(9)
5′-AUGGCCTTTCCGTGCCAAGG-3′;
(10)
5′-UCCGGCCTCGGAAGCUCUCU-3′;
(11)
5′-GCAUUCCGTGCGGAAGCCUU-3′;
(12)
5′-GGCCGAACTTTCCCGCCUUA-3′;
(13)
5′-GGUCUTGGCTTCGTGGAGCA-3′;
(14)
5′-GGAGCTTCGAGGCCCCAGGC-3′;
(15)
5′-GGUGGTCCACAACCCCUUUC-3′;
(16)
5′-CAUUAGGTGCAGAAAUCUUC-3′;
(17)
5′-UUCUGGGGACTTCCAGUUUA-3′;
(18)
5′-UGAUUCCAAAGCCAGGGUUA-3′;
(19)
5′-CUUUAGTCGTAGTTGCUUCC-3′;
(20)
5′-UUAAATAATCTAGTTUGAAG-3′;
(21)
5′-GUGUCCTTCATGCTTUGGAU-3′;
(22)
5′-AGAAAGAAGCAAAGAUUCAA-3′;
(23)
5′-AGAUUATCTTCTTTTAAUUU-3′;
(24)
5′-AAAAGATTATCTTCTUUUAA-3′;
(25)
5′-GAAAAGATTATCTTCUUUUA-3′;
(26)
5′-UGUGAAAAGATTATCUUCUU-3′;
(27)
5′-CUUGUGAAAAGATTAUCUUC-3′;
(28)
5′-GGUGGCCACAGGCAACGUCA-3′;
(29)
5′-CCAUGTCCCAGGCCTCCAGU-3′;
(30)
5′-GCAGUCTCCATGTCCCAGGC-3′;
(31)
5′-AGCAGTCTCCATGTCCCAGG-3′;
(32)
5′-AGUGGCACATACCACACCCU-3′;
(33)
5′-AUUUCCACATGCCCAGUGUU-3′;
(34)
5′-GGUCCCATCCCTTCTGCUGC-3′;
(35)
5′-UCUGGTCCCATCCCTUCUGC-3′;
(36)
5′-GGGUCTCCTCCACACCCUUC-3′;
(37)
5′-GCAAGGCAGAGAAACUCCAG-3′;
(38)
5′-GAUGGTTCCAGTCCCUCUUC-3′;
(39)
5′-UGUUUCCCCGGAGAGCAAUG-3′;
(40)
5′-AGCCGAACAGAAGGAGCGUC-3′;
(41)
5′-GCGUAGTTTCTCTTCCUCCC-3′;
(42)
5′-GCGGUATCCATGTCCCAGGC-3′;
(43)
5′-GCGGUATACAGGTCCCAGGC-3′;
(44)
5′-GCUGUTTCCATGTCCCAGGC-3′;
(45)
5′-GCUGUGTCCATGTCCCAGGC-3′;
(46)
5′-GCCGUTTCCATGTCCCAGGC-3′;
(47)
5′-GCCGUGTCCATGTCCCAGGC-3′;
(48)
5′-GCGGUATCCATAGTCUCCAU-3′;
(49)
5′-GCGGUATCCATCAGAUAUCG-3′;
(50)
5′-CUUUAGTCGTAGTTGUCUCU-3′;
(51)
5′-UCCGGGTCGTAGTTGCUUCC-3′;
(52)
5′-UCCGGCCTCGGAGTCUCCAU-3′;
(53)
5′-UCCGGCCTCGGGAGAUCUCU-3′;
(54)
5′-GGUATCCCCCCCCCCCCCCC-3′;
(55)
5′-GGAUUAAAACAGATTAAUAC-3′;
(56)
5′-GGUAU-3′;
(57)
5′-GGUA-3′;
(58)
5′-GUAU-3′;
(59)
5′-GGU-3′;
(60)
5′-GUA-3′;
(61)
5′-TGTCTG-3′;
(62)
5′-GTCT-3′;
(63)
5′-TCTCCG-3′;
(64)
5′-CTCC-3′;
(65)
5′-[G/A][A/C]AG[G/C][T/C]T[C/A];
(66)
5′-AAAGGTTA-3′;
(67)
5′-GAAGCTTC-3′;
(68)
5′-GCAGGCTC-3′;
(69)
5′-A[G/A]GGTT-3′;
(70)
5′-AGGGTT-3′;
(71)
5′-AAGGTT-3′;
(72)
5′-GGTT-3′;
(73)
5′-[A/G]GCT[T/C][T/C][G/C][T/A]-3′;
(74)
5′-AGCTTCCT-3′;
(75)
5′-AGCTTCGA-3′;
(76)
5′-GGCTTCGT-3′;
(77)
5′-TGCTTCCT-3′;
(78)
5′-AGCTCTCT-3′;
(79)
5′-G[G/C]TT-3′;
(80)
5′-GCTT-3′;
(81)
5′-CGGAGGTCTTGGCTTCGTGG-3′;
(82)
5′-AGGTCTTGGCTTCGTGGAGC-3′;
(83)
5′-GGGAAAGGTTATGCAAGGTC-3′;
(84)
5′-CTGTGATCTTGACATGCTGC-3′;
(85)
5′-ACTGACTGTCTTGAGGGTTC-3′;
(86)
5′-GCGTGTCTGGAAGCTTCCTT-3′;
(87)
5′-GAGTCTCTGGAGCTTCCTCT-3′;
(88)
5′-AGTCGTAGTTGCTTCCTAAC-3′;
(89)
5′-GTCTTGGCTTCGTGGAGCAG-3′;
(90)
5′-TTGGCTCGGCTTGCCTACTT-3′;
(91)
5′-ACAGTGTTGAGATACTCGGG-3′;
(92)
5′-TCGCACTTCAGTCTGAGCAG-3′;
(93)
5′-GGTGTCCTTGCACGTGGCTT-3′;
(94)
5′-TTTGCACACTTCGTACCCAA-3′;
(95)
5′-GCTGACAAAGATTCACTGGT-3′;
(96)
5′-GCGGAGGTCTTGGCTTCGTG-3′;
(97)
5′-CCAAGATCAGCAGTCT-3′;
(98)
5′-CTTGAAGCATCGTATC-3′;
(99)
5′-GCACACTTCGTACCCA-3′;
(100)
5′-GATAGCACCTTCAGCA-3′;
(101)
5′-CGTATTATAGCCGATT-3′;
(102)
5′-GCAGGCTCAGTGATGT-3′;
(103)
5′-GAAAGGTTATGCAAGG-3′;
(104)
5′-ATGGCCTCCCATCTCC-3′;
(105)
5′-CGCTTTTCTGTCTGGT-3′;
(106)
5′-GTGTCTGGAAGCTTCC-3′;
(107)
5′-TGGCCTCCCATCTCCT-3′;
(108)
5′-ATCTGGCAGCCCATCA-3′;
(109)
5′-GAGGTCTTGGCTTCGT-3′;
(110)
5′-ACACTTCGTGGGGTCC-3′;
(111)
5′-TTCGTGGGGTCCTTTT-3′;
(112)
5′-CCACTTGGCAGACCAT-3′;
(113)
5′-CCATCCATGAGGTCCT-3′;
(114)
5′-TCCAACACTTCGTGGG-3′;
(115)
5′-TCTTCATCGGCCCTGC-3′;
(116)
5′-CCAGCAGGTCAGCAAA-3′;
(117)
5′-CGCTTTTCTCTCCGGT-3′;
(118)
5′-GGUAUAGGACTCCAGATGUUUCC-3′;
(119) a variant of the motifs or sequences of (1) to (118) having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit cGAS activity, and
iv) selecting an oligonucleotide which inhibits cGAS activity.

2. A method for increasing the cGAS inhibitory activity of an oligonucleotide, the method comprising modifying the oligonucleotide such that the modified oligonucleotide comprises a motif including a sequence selected from the group consisting of:
(1)
5′-[A/G]GU[A/C][U/C]C-3′;
(2)
5′-A[G/A][U/G]C[U/C]C-3′;
(3)
5′-A[G/A][U/G]C[U/C]C[U/C][C/A]U-3′;
(4)
5′-GGUAUA-3′;
(5)
5′-UGUUUC-3′;
(6)
5′-UGUGUC-3′;
(7)
5′-CGUUUC-3′;
(8)
5′-CGUGUC-3′;
(9)
5′-AUGGCCTTTCCGTGCCAAGG-3′;
(10)
5′-UCCGGCCTCGGAAGCUCUCU-3′;
(11)
5′-GCAUUCCGTGCGGAAGCCUU-3′;
(12)
5′-GGCCGAACTTTCCCGCCUUA-3′;
(13)
5′-GGUCUTGGCTTCGTGGAGCA-3′;
(14)
5′-GGAGCTTCGAGGCCCCAGGC-3′;
(15)
5′-GGUGGTCCACAACCCCUUUC-3′;
(16)
5′-CAUUAGGTGCAGAAAUCUUC-3′;
(17)
5′-UUCUGGGGACTTCCAGUUUA-3′;
(18)
5′-UGAUUCCAAAGCCAGGGUUA-3′;
(19)
5′-CUUUAGTCGTAGTTGCUUCC-3′;
(20)
5′-UUAAATAATCTAGTTUGAAG-3′;
(21)
5′-GUGUCCTTCATGCTTUGGAU-3′;
(22)
5′-AGAAAGAAGCAAAGAUUCAA-3′;
(23)
5′-AGAUUATCTTCTTTTAAUUU-3′;
(24)
5′-AAAAGATTATCTTCTUUUAA-3′;
(25)
5′-GAAAAGATTATCTTCUUUUA-3′;
(26)
5′-UGUGAAAAGATTATCUUCUU-3′;
(27)
5′-CUUGUGAAAAGATTAUCUUC-3′;
(28)
5′-GGUGGCCACAGGCAACGUCA-3′;
(29)
5′-CCAUGTCCCAGGCCTCCAGU-3′;
(30)
5′-GCAGUCTCCATGTCCCAGGC-3′;
(31)
5′-AGCAGTCTCCATGTCCCAGG-3′;
(32)
5′-AGUGGCACATACCACACCCU-3′;
(33)
5′-AUUUCCACATGCCCAGUGUU-3′;
(34)
5′-GGUCCCATCCCTTCTGCUGC-3′;
(35)
5′-UCUGGTCCCATCCCTUCUGC-3′;
(36)
5′-GGGUCTCCTCCACACCCUUC-3′;
(37)
5′-GCAAGGCAGAGAAACUCCAG-3′;
(38)
5′-GAUGGTTCCAGTCCCUCUUC-3′;
(39)
5′-UGUUUCCCCGGAGAGCAAUG-3′;
(40)
5′-AGCCGAACAGAAGGAGCGUC-3′;
(41)
5′-GCGUAGTTTCTCTTCCUCCC-3′;
(42)
5′-GCGGUATCCATGTCCCAGGC-3′;
(43)
5′-GCGGUATACAGGTCCCAGGC-3′;
(44)
5′-GCUGUTTCCATGTCCCAGGC-3′;
(45)
5′-GCUGUGTCCATGTCCCAGGC-3′;
(46)
5′-GCCGUTTCCATGTCCCAGGC-3′;
(47)
5′-GCCGUGTCCATGTCCCAGGC-3′;
(48)
5′-GCGGUATCCATAGTCUCCAU-3′;
(49)
5′-GCGGUATCCATCAGAUAUCG-3′;
(50)
5′-CUUUAGTCGTAGTTGUCUCU-3′;
(51)
5′-UCCGGGTCGTAGTTGCUUCC-3′;
(52)
5′-UCCGGCCTCGGAGTCUCCAU-3′;
(53)
5′-UCCGGCCTCGGGAGAUCUCU-3′;
(54)
5′-GGUATCCCCCCCCCCCCCCC-3′;
(55)
5′-GGAUUAAAACAGATTAAUAC-3′;
(56)
5′-GGUAU-3′;
(57)
5′-GGUA-3′;
(58)
5′-GUAU-3′;
(59)
5′-GGU-3′;
(60)
5′-GUA-3′;
(61)
5′-TGTCTG-3′;
(62)
5′-GTCT-3′;
(63)
5′-TCTCCG-3′;
(64)
5′-CTCC-3′;
(65)
5′-[G/A][A/C]AG[G/C][T/C]T[C/A];
(66)
5′-AAAGGTTA-3′;
(67)
5′-GAAGCTTC-3′;
(68)
5′-GCAGGCTC-3′;
(69)
5′-A[G/A]GGTT-3′;
(70)
5′-AGGGTT-3′;
(71)
5′-AAGGTT-3′;
(72)
5′-GGTT-3′;
(73)
5′-[A/G]GCT[T/C][T/C][G/C][T/A]-3′;
(74)
5′-AGCTTCCT-3′;
(75)
5′-AGCTTCGA-3′;
(76)
5′-GGCTTCGT-3′;
(77)
5′-TGCTTCCT-3′;
(78)
5′-AGCTCTCT-3′;
(79)
5′-G[G/C]TT-3′;
(80)
5′-GCTT-3′;
(81)
5′-CGGAGGTCTTGGCTTCGTGG-3′;
(82)
5′-AGGTCTTGGCTTCGTGGAGC-3′;
(83)
5′-GGGAAAGGTTATGCAAGGTC-3′;
(84)
5′-CTGTGATCTTGACATGCTGC-3′;
(85)
5′-ACTGACTGTCTTGAGGGTTC-3′;
(86)
5′-GCGTGTCTGGAAGCTTCCTT-3′;
(87)
5′-GAGTCTCTGGAGCTTCCTCT-3′;
(88)
5′-AGTCGTAGTTGCTTCCTAAC-3′;
(89)
5′-GTCTTGGCTTCGTGGAGCAG-3′;
(90)
5′-TTGGCTCGGCTTGCCTACTT-3′;
(91)
5′-ACAGTGTTGAGATACTCGGG-3′;
(92)
5′-TCGCACTTCAGTCTGAGCAG-3′;
(93)
5′-GGTGTCCTTGCACGTGGCTT-3′;
(94)
5′-TTTGCACACTTCGTACCCAA-3′;
(95)
5′-GCTGACAAAGATTCACTGGT-3′;
(96)
5′-GCGGAGGTCTTGGCTTCGTG-3′;
(97)
5′-CCAAGATCAGCAGTCT-3′;
(98)
5′-CTTGAAGCATCGTATC-3′;
(99)
5′-GCACACTTCGTACCCA-3′;
(100)
5′-GATAGCACCTTCAGCA-3′;
(101)
5′-CGTATTATAGCCGATT-3′;
(102)
5′-GCAGGCTCAGTGATGT-3′;
(103)
5′-GAAAGGTTATGCAAGG-3′;
(104)
5′-ATGGCCTCCCATCTCC-3′;
(105)
5′-CGCTTTTCTGTCTGGT-3′;
(106)
5′-GTGTCTGGAAGCTTCC-3′;
(107)
5′-TGGCCTCCCATCTCCT-3′;
(108)
5′-ATCTGGCAGCCCATCA-3′;
(109)
5′-GAGGTCTTGGCTTCGT-3′;
(110)
5′-ACACTTCGTGGGGTCC-3′;
(111)
5′-TTCGTGGGGTCCTTTT-3′;
(112)
5′-CCACTTGGCAGACCAT-3′;
(113)
5′-CCATCCATGAGGTCCT-3′;
(114)
5′-TCCAACACTTCGTGGG-3′;
(115)
5′-TCTTCATCGGCCCTGC-3′;
(116)
5′-CCAGCAGGTCAGCAAA-3′;
(117)
5′-CGCTTTTCTCTCCGGT-3′;
(118)
5′-GGUAUAGGACTCCAGATGUUUCC-3′;
(119) a variant of the motifs or sequences of (1) to (118) having at least about 75% sequence identity thereto;
wherein the U may be a T and/or the T may be a U.

3. The method of claim 2, wherein the step of modifying the oligonucleotide includes adding a sequence of nucleotides to the 5′ and/or 3′ end of the oligonucleotide such that the modified oligonucleotide comprises the motif.

4. The method of claim 2 or 3, which further comprises testing the ability of the modified oligonucleotide to inhibit cGAS activity, and selecting an oligonucleotide which inhibits cGAS activity to a greater extent than the unmodified oligonucleotide.

5. The method according to any one of the preceding claims, wherein the oligonucleotide does not bind or is not designed to bind a transcript that encodes cGAS or a complement thereof.

6. The method according to any one of the preceding claims, wherein the oligonucleotide binds or is designed to bind a target transcript that does not encode cGAS or a complement thereof.

7. The method according to any one of the preceding claims, wherein the motif is within eleven bases of the 5′ and/or 3′ end of the oligonucleotide.

8. The method of according to any one of the preceding claims, wherein the motif is within eight bases of the 5′ and/or 3′ end of the oligonucleotide.

9. The method according to any one of the preceding claims, wherein the motif is at or towards the 5′ and/or 3′ end of the oligonucleotide.

10. The method according to any one of the preceding claims, wherein the motif has the sequence 5′-GGUAUC-3′, 5′-AGUCUC-3′, 5′-GGUCCC-3′, 5′-GGUCUC-3′, 5′-AAGCUC-3′, 5′-AGUCCC-3′, 5′-GGUAUA-3′, 5′-UGUUUC-3′, 5′-UGUGUC-3′, 5′-CGUUUC-3′, 5′-CGUGUC-3′, 5′-GGUAU-3′, 5′-GGUA-3′, 5′-GGU-3′, 5′-TGTCTG-3′, 5′-GTCT-3′, 5′-CTCC-3′, 5′-AAAGGTTA-3′, 5′-GAAGCTTC-3′, 5′-GCAGGCTC-3′, 5′-AGGGTT-3′, 5′-AAGGTT-3′, 5′-GGTT-3′, 5′-AGCTTCCT-3′, 5′-AGCTTCGA-3′, 5′-GGCTTCGT-3′, 5′-TGCTTCCT-3′, 5′-AGCTCTCT-3′ or 5′-GCTT-3′, wherein the U may be a T.

11. The method according to any one of the preceding claims, wherein the motif has the sequence of 5′-GGUAUC-3′, 5′-GGUATC-3′, 5′-AGUCTC-3′, 5′-AGTCTC-3′, 5′-GGUCCC-3′, 5′-GGUCTC-3′, 5′-AAGCUC-3′, 5′-AGTCCC-3′, 5′-GGUATA-3′, 5′-UGUTTC-3′, 5′-UGUGTC-3′, 5′-CGUTTC-3′, 5′-CGUGTC-3′, 5′-GGUAU-3′, 5′-GGUAT-3′, 5′-GGUA-3′, 5′-GGU-3′, 5′-TGTCTG-3′, 5′-GTCT-3′, 5′-TCTCCG-3′, 5′-CTCC-3′, 5′-AAAGGTTA-3′, 5′-GAAGCTTC-3′, 5′-GCAGGCTC-3′, 5′-AGGGTT-3′, 5′-AAGGTT-3′, 5′-GGTT-3′, 5′-AGCTTCCT-3′, 5′-AGCTTCGA-3′, 5′-GGCTTCGT-3′, 5′-TGCTTCCT-3′, 5′-AGCTCTCT-3′ or 5′-GCTT-3′.

12. The method according to any one of the preceding claims, wherein one or more of the bases of the motif are a modified base and/or have a modified backbone.

13. The method according to any one of the preceding claims, wherein the motif has the sequence of 5′-mGmGmUATC-3′, 5′-mAmGmUCTC-3′, 5′-mAmGTCTC-3′, 5′-mGmGmUmCmCC-3′, 5′-mGmGmUmCTC-3′, 5′-AAGCmUmC-3′, 5′-AGTCCC-3′, 5′-mGmGmUATA-3′, 5′-mUmGmUTTC-3′, 5′-mUmGmUGTC-3′, 5′-mCmGmUTTC-3′, 5′-mCmGmUGTC-3′, 5′-mGmGmUAU-3′, 5′-mGmGmUAT-3′, 5′-mGmGmUmAU-3′, 5′-mGmGmUmAT-3′, 5′-mGmGmUmAmU-3′, 5′-mGmGmUA-3′, 5′-mGmGmUmA-3′, 5′-mGmGmU-3′, 5′-mTmGTCTG-3′, 5′-TGTCTmG-3′, 5′-mGTCT-3′, 5′-GTCT-3′, 5′-TCTCCG-3′, 5′-CTCC-3′, 5′-mAmAAGGTTA-3′, 5′-GAAGCTmTmC-3′, 5′-mGmCmAGGCTC-3′, 5′-mAAGGTT-3′, 5′-AGmGmGmTmT-3′, 5′-AGCTmTmCmCmT-3′, 5′-AGCTTmCmCmT-3′, 5′-mAmGmCTTCGA-3′, 5′-GGCTTmCmGmT-3′, 5′-GGCTTCGT-3′, 5′-TGCTTCmCmT-3′ or 5′-AGCmTmCmTmCmT-3′, wherein m is a modified base and/or has a modified backbone.

14. A method for selecting or designing an oligonucleotide which does not inhibit cGAS activity, the method comprising
i) scanning a polynucleotide, or complement thereof, for a region having a motif including a sequence selected from the group consisting of:
(1)
5′-[C/U]CUUCU-3′;
(2)
5′-CACCCTTCTCTCTGGUCCCA-3′;
(3)
5′-CCUUCTCTCTGGTCCCAUCC-3′;
(4)
5′-UCUCUGGTCCCATCCCUUCU-3′;
(5)
5′-AUAUCTGCTGCCCACCUUCU-3′;
(6)
5′-GUCCCATCCCTTCTGCUGCC-3′;
(7)
5′-GUCUCCTCCACACCCUUCUC-3′;
(8)
5′-CAGGCCTCCAGTGTCUUCUC-3′;
(9)
5′-UCCCAACTCTTCTAACUCGU-3′;
(10) a variant of the motifs or sequences of (1) to (9) having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit cGAS activity, and
iv) selecting an oligonucleotide which does not inhibit cGAS activity.

15. A method for reducing the cGAS inhibitory activity of an oligonucleotide, the method comprising modifying the oligonucleotide such that the modified oligonucleotide comprises a motif including a sequence selected from the group consisting of:
(1)
5′-[C/U]CUUCU-3′;
(2)
5′-CACCCTTCTCTCTGGUCCCA-3′;
(3)
5′-CCUUCTCTCTGGTCCCAUCC-3′;
(4)
5′-UCUCUGGTCCCATCCCUUCU-3′;
(5)
5′-AUAUCTGCTGCCCACCUUCU-3′;
(6)
5′-GUCCCATCCCTTCTGCUGCC-3′;
(7)
5′-GUCUCCTCCACACCCUUCUC-3′;
(8)
5′-CAGGCCTCCAGTGTCUUCUC-3′;
(9)
5′-UCCCAACTCTTCTAACUCGU-3′;
(10) a variant of the motifs or sequences of (1) to (9) having at least about 75% sequence identity thereto;
wherein the U may be a T and/or the T may be a U.

16. The method of claim 15, wherein the step of modifying the oligonucleotide includes adding a sequence of nucleotides to the 5′ and/or 3′ end of the oligonucleotide such that the modified oligonucleotide comprises the motif.

17. The method of claim 15 or 16, which further comprises testing the ability of the modified oligonucleotide to inhibit cGAS activity, and selecting an oligonucleotide which inhibits cGAS activity to a lesser extent than the unmodified oligonucleotide.

18. The method according to any one of claims 14 to 17, wherein the motif is within thirteen bases of the 5′ and/or 3′ end of the oligonucleotide.

19. The method according to any one of claims 14 to 18, wherein the motif is within nine bases of the 5′ and/or 3′ end of the oligonucleotide.

20. The method according to any one of claims 14 to 19, wherein the motif is at or towards the 5′ and/or 3′ end of the oligonucleotide.

21. The method according to any one of claims 14 to 20, wherein the motif has the sequence 5′-CCUUCU-3′ or 5′-UCUUCU-3′, wherein the U may be a T.

22. The method according to any one of claims 14 to 21, wherein one or more of the bases of the motif are a modified base and/or have a modified backbone.

23. The method according to any one of claims 14 to 22, wherein the motif has the sequence 5′-mCmCUUCU-3′, 5′-mCmCmUmUmCU-3′, 5′-CmCmUmUmCmU-3′, 5′-CCUUCU-3′, 5′-CCmUmUmCmU-3′, 5′-UCmUmUmCmU-3′ or 5′-UCUUCU-3′, wherein the U may be a T and wherein m is a modified base and/or has a modified backbone.

24. A method for selecting or designing an oligonucleotide which inhibits TLR9 activity, the method comprising
i) scanning a polynucleotide, or complement thereof, for a region having a motif including a sequence selected from the group consisting of:
(1)
5′-G[G/C]CCT[C/G]-3′;
(2)
5′-CUU-3′, wherein the motif is within 10 bases
of the 5′ and/or 3′ end of the oligonucleotide;
(3)
5′-CUUGUGAAAAGATTAUCUUC-3′;
(4)
5′-CUUCUCTCTGGTCCCAUCCC-3′;
(5)
5′-CCUUCTCTCTGGTCCCAUCC-3′;
(6)
5′-CCCUUCTCTCTGGTCCCAUC-3′;
(7)
5′-ACCCUTCTCTCTGGTCCCAU-3′;
(8)
5′-CUUCCACAATCAAGACAUUC-3′;
(9)
5′-CUUCGTGGGGTCCTTUUCAC-3′;
(10)
5′-CACUUCGTGGGGTCCUUUUC-3′;
(11)
5′-CCAACACTTCGTGGGGUCCU-3′;
(12)
5′-UCCGGCCTCGGAAGCUCUCU-3′;
(13)
5′-CCAUGTCCCAGGCCTCCAGU-3′;
(14)
5′-UUGGCCTGTGGATGCUUUGU-3′;
(15)
5′-AAAUGTCCTGGCCCTCACUG-3′;
(16)
5′-UCCGGCCTCGGAGTCUCCAU-3′;
(17)
5′-UCCGGCCTCGGCAGAUAUCG-3′;
(18)
5′-GGCCGAACTTTCCCGCCUUA-3′;
(19)
5′-GGUCUTGGCTTCGTGGAGCA-3′;
(20)
5′-GGAGCTTCGAGGCCCCAGGC-3′;
(21)
5′-GGUGGTCCACAACCCCUUUC-3′;
(22)
5′-CAUUAGGTGCAGAAAUCUUC-3′;
(23)
5′-UUCUGGGGACTTCCAGUUUA-3′;
(24)
5′-UGAUUCCAAAGCCAGGGUUA-3′;
(25)
5′-UCCGGGTCGTAGTTGCUUCC-3′;
(26)
5′-CCUAGAAAGAAGCAAAGAUU-3′;
(27)
5′-GAUUAAAACAGATTAAUACA-3′;
(28)
5′-GGAUUAAAACAGATTAAUAC-3′;
(29)
5′-AAUUUAAAGCATGAAUAUUA-3′;
(30)
5′-GCGUAGTTTCTCTTCCUCCC-3′
(31)
5′-UGACAAAACAATAATAACAG-3′;
(32)
5′-ACA-3′, wherein the motif is at or towards
the 5′ end of the oligonucleotide;
(33) 5′-CAC-3′, wherein the motif is at or
towards the 5′ end of the oligonucleotide;
(34)
5′-ACACTTCGTGGGGTCCTTTT-3′;
(35)
5′-GCGTGTCTGGAAGCTTCCTT-3′;
(36)
5′-TCAAAGGACTGAGGAAAGGG-3′;
(37)
5′-ATCCAACACTTCGTGGGGTC-3′;
(38)
5′-GCCCATCCATGAGGTCCTGG-3′;
(39)
5′-GGGTATCGAAAGAGTCTGGA-3′;
(40)
5′-GGTTTTGGCTGGGATCAAGT-3′;
(41)
5′-GCGACTATACGCGCAATATG-3′;
(42)
5′-ACTGACTGTCTTGAGGGTTC-3′;
(43)
5′-AACACTTCGTGGGGTCCTTT-3′;
(44)
5′-GTCCAAGATCAGCAGTCTCA-3′;
(45)
5′-ACAGTGTTGAGATACTCGGG-3′;
(46)
5′-TGGGCTGGAATCCGAGTTAT-3′;
(47)
5′-CGGCATCCACCACGTCGTCC-3′;
(48)
5′-GCGTATTATAGCCGATTAAC-3′;
(49)
5′-GGAGGTCTTGGCTTCGTGGA-3′;
(50)
5′-TGGGTTACGGCTCAGTATGG-3′;
(51)
5′-CCGCCATGTTTCTTCTTGGA-3′;
(52)
5′-AGCTTCGAGGCCCCAG-3′;
(53)
5′-GCCATGTTTCTTCTTG-3′;
(54)
5′-CACTTCGTGGGGTCCT-3′;
(55)
5′-CGGCCTCGGAAGCTCT-3′;
(56)
5′-ACACTTCGTGGGGTCC-3′;
(57)
5′-TGCACACTTCGTACCC-3′;
(58)
5′-CCACATCCTGTGGCTC-3′;
(59)
5′-CTGCAGCTTCCTTGTC-3′;
(60)
5′-ACTTCGTGGGGTCCTT-3′;
(61)
5′-CCCACTTGGCAGACCA-3′;
(62)
5′-GTCCCCTGTTGACTGG-3′;
(63)
5′-ACGTTCAGTCCTGTCC-3′;
(64)
5′-GGTCATTACAATAGCT-3′;
(65)
5′-TCGTGGGGTCCTTTTC-3′;
(66)
5′-GTGTCTGGAAGCTTCC-3′;
(67)
5′-ATGGCCTCCCATCTCC-3′;
(68)
5′-TGCTCCTCGGTCTCCC-3′;
(69)
5′-GCATCCACCACGTCGT-3′;
(70)
5′-CTTCGTGGGGTCCTTT-3′;
(71)
5′-AGGCCCTTCGCACTTC-3′;
(72)
5′-GCGGUATCCATGTCCCAGGC-3′;
(73)
5′-GCUGUTTCCATGTCCCAGGC-3′;
(74)
5′-GCUGUGTCCATGTCCCAGGC-3′
(75)
5′-GCCGUTTCCATGTCCCAGGC-3′;
(76)
5′-GCGGUATCC-3′;
(77)
5′-GCUGUTTCC-3′;
(78)
5′-GCUGUGTCC-3′;
(79)
5′-GCCGUTTCC-3′;
(80)
5′-CUUCGTGGGGTCCTTUUCAC;
(81)
5′-CUUCGTGGG-3′;
(82)
5′-UCG-3′;
(83)
5′-ACG-3′;
(84)
5′-ACC-3′;
(85)
5′-CGC-3′;
(86)
5′-GAU-3′;
(87)
5′-GGG-3′;
(88)
5′-AGC-3′;
(89)
5′-UUC-3′;
(90)
5′-UUG-3′;
(91)
5′-CAC-3′;
and
and
(92) a variant of the motifs or sequences of (1) to (91) having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR9 activity, and
iv) selecting an oligonucleotide which inhibits TLR9 activity.

25. A method for increasing the TLR9 inhibitory activity of an oligonucleotide, the method comprising modifying the oligonucleotide such that the modified oligonucleotide comprises a motif including a sequence selected from the group consisting of:
(1)
5′-G[G/C]CCT[C/G]-3′;
(2)
5′-CUU-3′, wherein the motif is within 10 bases
of the 5′ and/or 3′ end of the oligonucleotide;
(3)
5′-CUUGUGAAAAGATTAUCUUC-3′;
(4)
5′-CUUCUCTCTGGTCCCAUCCC-3′;
(5)
5′-CCUUCTCTCTGGTCCCAUCC-3′;
(6)
5′-CCCUUCTCTCTGGTCCCAUC-3′;
(7)
5′-ACCCUTCTCTCTGGTCCCAU-3′;
(8)
5′-CUUCCACAATCAAGACAUUC-3′;
(9)
5′-CUUCGTGGGGTCCTTUUCAC-3′;
(10)
5′-CACUUCGTGGGGTCCUUUUC-3′;
(11)
5′-CCAACACTTCGTGGGGUCCU-3′;
(12)
5′-UCCGGCCTCGGAAGCUCUCU-3′;
(13)
5′-CCAUGTCCCAGGCCTCCAGU-3′;
(14)
5′-UUGGCCTGTGGATGCUUUGU-3′;
(15)
5′-AAAUGTCCTGGCCCTCACUG-3′;
(16)
5′-UCCGGCCTCGGAGTCUCCAU-3′;
(17)
5′-UCCGGCCTCGGCAGAUAUCG-3′;
(18)
5′-GGCCGAACTTTCCCGCCUUA-3′;
(19)
5′-GGUCUTGGCTTCGTGGAGCA-3′;
(20)
5′-GGAGCTTCGAGGCCCCAGGC-3′;
(21)
5′-GGUGGTCCACAACCCCUUUC-3′;
(22)
5′-CAUUAGGTGCAGAAAUCUUC-3′;
(23)
5′-UUCUGGGGACTTCCAGUUUA-3′;
(24)
5′-UGAUUCCAAAGCCAGGGUUA-3′;
(25)
5′-UCCGGGTCGTAGTTGCUUCC-3′;
(26)
5′-CCUAGAAAGAAGCAAAGAUU-3′;
(27)
5′-GAUUAAAACAGATTAAUACA-3′;
(28)
5′-GGAUUAAAACAGATTAAUAC-3′;
(29)
5′-AAUUUAAAGCATGAAUAUUA-3′;
(30)
5′-GCGUAGTTTCTCTTCCUCCC-3′;
(31)
5′-UGACAAAACAATAATAACAG-3′;
(32)
5′-ACA-3′, wherein the motif is at or towards
the 5′ end of the oligonucleotide;
(33)
5′-CAC-3′, wherein the motif is at or towards
the 5′ end of the oligonucleotide;
(34)
5′-ACACTTCGTGGGGTCCTTTT-3′;
(35)
5′-GCGTGTCTGGAAGCTTCCTT-3′;
(36)
5′-TCAAAGGACTGAGGAAAGGG-3′;
(37)
5′-ATCCAACACTTCGTGGGGTC-3′;
(38)
5′-GCCCATCCATGAGGTCCTGG-3′;
(39)
5′-GGGTATCGAAAGAGTCTGGA-3′;
(40)
5′-GGTTTTGGCTGGGATCAAGT-3′;
(41)
5′-GCGACTATACGCGCAATATG-3′;
(42)
5′-ACTGACTGTCTTGAGGGTTC-3′;
(43)
5′-AACACTTCGTGGGGTCCTTT-3′;
(44)
5′-GTCCAAGATCAGCAGTCTCA-3′;
(45)
5′-ACAGTGTTGAGATACTCGGG-3′;
(46)
5′-TGGGCTGGAATCCGAGTTAT-3′;
(47)
5′-CGGCATCCACCACGTCGTCC-3′;
(48)
5′-GCGTATTATAGCCGATTAAC-3′;
(49)
5′-GGAGGTCTTGGCTTCGTGGA-3′;
(50)
5′-TGGGTTACGGCTCAGTATGG-3′;
(51)
5′-CCGCCATGTTTCTTCTTGGA-3′;
(52)
5′-AGCTTCGAGGCCCCAG-3′;
(53)
5′-GCCATGTTTCTTCTTG-3′;
(54)
5′-CACTTCGTGGGGTCCT-3′;
(55)
5′-CGGCCTCGGAAGCTCT-3′;
(56)
5′-ACACTTCGTGGGGTCC-3′;
(57)
5′-TGCACACTTCGTACCC-3′;
(58)
5′-CCACATCCTGTGGCTC-3′;
(59)
5′-CTGCAGCTTCCTTGTC-3′;
(60)
5′-ACTTCGTGGGGTCCTT-3′;
(61)
5′-CCCACTTGGCAGACCA-3′;
(62)
5′-GTCCCCTGTTGACTGG-3′;
(63)
5′-ACGTTCAGTCCTGTCC-3′;
(64)
5′-GGTCATTACAATAGCT-3′;
(65)
5′-TCGTGGGGTCCTTTTC-3′;
(66)
5′-GTGTCTGGAAGCTTCC-3′;
(67)
5′-ATGGCCTCCCATCTCC-3′;
(68)
5′-TGCTCCTCGGTCTCCC-3′;
(69)
5′-GCATCCACCACGTCGT-3′;
(70)
5′-CTTCGTGGGGTCCTTT-3′;
(71)
5′-AGGCCCTTCGCACTTC-3′;
(72)
5′-GCGGUATCCATGTCCCAGGC-3′;
(73)
5′-GCUGUTTCCATGTCCCAGGC-3′;
(74)
5′-GCUGUGTCCATGTCCCAGGC-3′
(75)
5′-GCCGUTTCCATGTCCCAGGC-3′;
(76)
5′-GCGGUATCC-3′;
(77)
5′-GCUGUTTCC-3′;
(78)
5′-GCUGUGTCC-3′;
(79)
5′-GCCGUTTCC-3′;
(80)
5′-CUUCGTGGGGTCCTTUUCAC;
(81)
5′-CUUCGTGGG-3′;
(82)
5′-UCG-3′;
(83)
5′-ACG-3′:
(84)
5′-ACC-3′;
(85)
5′-CGC-3′;
(86)
5′-GAU-3′;
(87)
5′-GGG-3′;
(88)
5′-AGC-3′;
(89)
5′-UUC-3′;
(90)
5′-UUG-3′;
(91)
5′-CAC-3′;
and
and
(92) a variant of the motifs or sequences of (1) to (91) having at least about 75% sequence identity thereto;
wherein the U may be a T and/or the T may be a U.

26. The method of claim 25, which further comprises testing the ability of the modified oligonucleotide to inhibit TLR9 activity, and selecting an oligonucleotide which inhibits TLR9 activity to a greater extent than the unmodified oligonucleotide.

27. The method of claim 25 or claim 26, wherein the step of modifying the oligonucleotide includes adding a sequence of nucleotides to the 5′ and/or 3′ end of the oligonucleotide such that the modified oligonucleotide comprises the motif.

28. The method according to any one of claims 24 to 27, wherein the oligonucleotide does not bind or is not designed to bind a transcript that encodes TLR9 or a complement thereof.

29. The method according to any one of claims 24 to 28, wherein the oligonucleotide binds or is designed to bind a target transcript that does not encode TLR9 or a complement thereof.

30. The method according to any one of claims 24 to 29, wherein the motif is within bases of the 5′ and/or 3′ end of the oligonucleotide.

31. The method according to any one of claims 24 to 30, wherein the motif is within bases of the 5′ and/or 3′ end of the oligonucleotide.

32. The method according to any one of claims 24 to 31, wherein the motif is at or towards the 5′ and/or 3′ end of the oligonucleotide.

33. The method according to any one of claims 24 to 32, wherein the motif is at or towards the 5′ end of the oligonucleotide.

34. The method according to any one of claims 24 to 33, wherein the motif has the sequence 5′-CUU-3′, 5′-CUT-3′, or 5′-CTT-3′.

35. The method according to any one of claims 24 to 33, wherein the motif has the sequence 5′-GGCCTC-3′, 5′-GGCCTG-3′, or 5′-GCCCTC-3′, wherein the T may be a U.

36. The method according to any one of claims 24 to 35, wherein one or more of the bases of the motif are a modified base and/or have a modified backbone.

37. The method according to any one of claims 24 to 34 or 36, wherein the motif has the sequence 5′-mCmUmU-3′ or 5′-mCmUT-3′, wherein m is a modified base and/or has a modified backbone.

38. The method according to any one of claims 24 to 33, 35 or 36, wherein the motif has the sequence 5′-mGmGCCTC-3′, 5′-GGCCTmC-3′, 5′-mGmGmCCTG-3′ or 5′-GCCCTmC-3′, wherein the T may be a U and wherein m is a modified base and/or has a modified backbone.

39. The method according to any one of the preceding claims, wherein the oligonucleotide comprises
a) a 5′ region comprising bases which are modified and/or which have a modified backbone,
b) a middle region comprising ribonucleic acid, deoxyribonucleic acid, or combination thereof, bases, which optionally have a modified backbone, and
c) a 3′ region comprising bases which are modified and/or which have a modified backbone.

40. The method of claim 39, wherein the middle region is about 10 bases in length.

41. The method of claim 39 or claim 40, wherein the 5′ region and/or the 3′ region are: (a) about 3 bases in length; or (b) about 5 bases in length.

42. A method for selecting or designing an oligonucleotide which inhibits TLR9 activity, the method comprising
i) scanning a polynucleotide, or complement thereof, for regions having at least about 50% adenine bases;
ii) producing one or more candidate oligonucleotides comprising a 5′ region, a 3′ region and a middle region comprising ribonucleic acid, deoxyribonucleic acid, or combination thereof, bases, which optionally have a modified backbone, wherein one or both of the 5′ region and the 3′ region comprise bases which are modified and/or which have a modified backbone, and wherein at least about 50% of the bases of the middle region are adenine bases;
iii) testing the ability of the one or more candidate oligonucleotides to inhibit TLR9 activity, and
iv) selecting an oligonucleotide which inhibits TLR9 activity.

43. The method of claim 42, wherein the oligonucleotide comprises a motif having a sequence of 5′-[T/G][A/T][G/A/T]AA[A/C][A/G][G/C/A]A[T/G][T/G/C]A[A/T]-3′, wherein the T may be a U.

44. The method according to claim 42 or claim 43, wherein the 5′ region and/or the 3′ region are about 5 bases in length and the middle region is about 10 bases in length, wherein the middle region comprises at least five adenine bases.

45. The method of claim 44, wherein two, three and/or four of the at least five adenine bases are in a continuous sequence.

46. The method according to any one of claims 42 to 45, wherein the oligonucleotide does not bind or is not designed to bind a transcript that encodes TLR9 or a complement thereof.

47. The method according to any one of claims 42 to 46, wherein the oligonucleotide binds or is designed to bind a target transcript that does not encode TLR9 or a complement thereof.

48. A method for selecting or designing an oligonucleotide which inhibits TLR7 activity, the method comprising
i) scanning a polynucleotide, or complement thereof, for a region having a motif including a sequence selected from the group consisting of:
(1)
5′-[A/G]GU[A/C][U/C]C-3′;
(2)
5′-A[G/A][U/G]C[U/C]C-3′;
(3)
5′-A[G/A][U/G]C[U/C]C[U/C][C/A]U-3′;
(4)
5′-GGUAUA-3′;
(5)
5′-UGUUUC-3′;
(6)
5′-UGUGUC-3′;
(7)
5′-CGUGUC-3′;
(8)
5′-GCAGUCTCCATGTCCCAGGC-3′;
(9)
5′-GAUGGTTCCAGTCCCUCUUC-3′;
(10)
5′-AGCAGTCTCCATGTCCCAGG-3′;
(11)
5′-GGGUCTCCTCCACACCCUUC-3′;
(12)
5′-GGUGGCCACAGGCAACGUCA-3′;
(13)
5′-GCCGUTTCCATGTCCCAGGC-3′;
(14)
5′-GCGGUATCCATGTCCCAGGC-3′;
(15)
5′-GCGGUATACAGGTCCCAGGC-3′;
(16)
5′-GCUGUTTCCATGTCCCAGGC-3′;
(17)
5′-GCUGUGTCCATGTCCCAGGC-3′;
(18)
5′-GCCGUGTCCATGTCCCAGGC-3′;
(19)
5′-GCGGUAUCCAUGUCCCAGGC-3′;
(20)
5′-GGUATCCCCCCCCCCCCCCC-3′;
(21)
5′-GUCCCATCCCTTCTGCUGCC-3′;
(22)
5′-UUCUCTCTGGTCCCAUCCCU-3′;
(23)
5′-GUUCAGTCAGATCGCUGGGA-3′;
(24)
5′-AUGACATTTCGTGGCUCCUA-3′;
(25)
5′-UCUCCATGTCCCAGGCCUCC-3′;
(26)
5′-AGUCUCCATGTCCCAGGCCU-3′;
(27)
5′-CCAUGTCCCAGGCCTCCAGU-3′;
(28)
5′-GCAAGGCAGAGAAACUCCAG-3′;
(29)
5′-GGAUUAAAACAGATTAAUAC-3′;
(30)
5′-AGCCGAACAGAAGGAGCGUC-3′;
(31)
5′-UCCGGCCTCGGAAGCUCUCU-3′;
(32)
5′-UCCGGCCTCGGAGTCUCCAU-3′;
(33)
5′-GCGGUATCCATAGTCUCCAU-3′;
(34)
5′-GAUUAAAACAGATTAAUACA-3′;
(35)
5′-UGACAAAACAATAATAACAG-3′;
(36)
5′-CCAACACTTCGTGGGGUCCU-3′;
(37)
5′-GUGUCCTTCATGCTTUGGAU-3′;
(38)
5′-GUCCCAGGCCTCCAGUGUCU-3′;
(39)
5′-UUGGCCTGTGGATGCUUUGU-3′;
(40)
5′-GUCCGTACCTCCACCCACCG-3′;
(41)
5′-GUGUUTTTAATTTTGUAGAG-3′;
(42)
5′-GUCAAACCTAGAAAGAAGCA-3′;
(43)
5′-GGUCUCCTCCACACCCUUCU-3′;
(44)
5′-GUCUCCTCCACACCCUUCUC-3′;
(45)
5′-UGAUGATGCTTGCAGGAGGC-3′;
(46)
5′-UUAAATAATCTAGTTUGAAG-3′;
(47)
5′-AAAGCAGTCTCCATGUCCCA-3′;
(48)
5′-GCGUAGTTTCTCTTCCUCCC-3′;
(49)
5′-UCUGGTCCCATCCCTUCUGC-3′;
(50)
5′-UAUUUCCACATGCCCAGUGU-3′;
(51)
5′-UGUUUCCCCGGAGAGCAAUG-3′;
(52)
5′-UUAGCTCCTTGCCTCGUUCC-3′;
(53)
5′-AUUUCCACATGCCCAGUGUU-3′;
(54)
5′-UGGCGTAGTTTCTCTUCCUC-3′;
(55)
5′-UGACATTTCGTGGCTCCUAC-3′;
(56)
5′-GAAAAGATTATCTTCUUUUA-3′;
(57)
5′-UGUGAAAAGATTATCUUCUU-3′;
(58)
5′-UUGUGAAAAGATTATCUUCU-3′;
(59)
5′-UUUGAAATTCAGAAGAUUUG-3′;
(60)
5′-AAGCAGTCTCCATGTCCCAG-3′;
(61)
5′-AGGAUTAAAACAGATUAAUA-3′;
(62)
5′-AGAUUATCTTCTTTTAAUUU-3′;
(63)
5′-UCCCAACTCTTCTAACUCGU-3′;
(64)
5′-UAAAATAAGGGGAATAGGGG-3′;
(65)
5′-AGUGGCACATACCACACCCU-3′;
(66)
5′-AAGAUTATCTTCTTTUAAUU-3′;
(67)
5′-AGAAAGAAGCAAAGAUUCAA-3′;
(68)
5′-UCCCATCCCTTCTGCUGCCA-3′;
(69)
5′-ACCCUTCTCTCTGGTCCCAU-3′;
(70)
5′-AAUAUCTGCTGCCCACCUUC-3′;
(71)
5′-UCUCUCTGGTCCCATCCCUU-3′;
(72)
5′-AGGCCTCCAGTGTCTUCUCC-3′;
(73)
5′-CAAGCCCCAGCGTTCCUCCG-3′;
(74)
5′-AAAUGTCCTGGCCCTCACUG-3′;
(75)
5′-AAUUUAAAGCATGAAUAUUA-3′;
(76)
5′-AAAAGATTATCTTCTUUUAA-3′;
(77)
5′-AUAUCTGCTGCCCACCUUCU-3′;
(78)
5′-CAGUCTCCATGTCCCAGGCC-3′;
(79)
5′-AAAGATTATCTTCTTUUAAU-3′;
(80)
5′-CCCUUCTCTCTGGTCCCAUC-3′;
(81)
5′-AUGGCCTTTCCGTGCCAAGG-3′;
(82)
5′-GCAUUCCGTGCGGAAGCCUU-3′;
(83)
5′-GGCCGAACTTTCCCGCCUUA-3′;
(84)
5′-GGUCUTGGCTTCGTGGAGCA-3′;
(85)
5′-GGAGCTTCGAGGCCCCAGGC-3′;
(86)
5′-GGUGGTCCACAACCCCUUUC-3′;
(87)
5′-UUCUGGGGACTTCCAGUUUA-3′;
(88)
5′-UGAUUCCAAAGCCAGGGUUA-3′;
(89)
5′-GGGUATCGAAAGAGTCUGGA-3′;
(90)
5′-CUUGCACGTGGCTTCGUCUC-3′;
(91)
5′-GUGUCCTTGCACGTGGCUUC-3′;
(92)
5′-GUAAAAAGCTTTTGAAGUGA-3′;
(93)
5′-AUGCCATCCACTTGAUAGGC-3′;
(94)
5′-UGAAGTAAAAATCAAUAGCG-3′;
(95)
5′-AAGGCCCTTCGCACTUCUUA-3′;
(96)
5′-GUACUCGTCGGCATCCACCA-3′;
(97)
5′-GUCCUTGCACGTGGCUUCGU-3′;
(98)
5′-GCCCATCCATGAGGTCCUGG-3′;
(99)
5′-GUAAAAGGAGAAAACUAUCU-3′;
(100)
5′-UUGAAGTGAAGTAAAAGGAG-3′;
(101)
5′-GUUACTCGTGCCTTGGCAAA-3′;
(102)
5′-GUCCAAGATCAGCAGUCUCA-3′;
(103)
5′-UUCAATGGGAGAATAAAGCA-3′;
(104)
5′-GCAAGGCCCTTCGCACUUCU-3′;
(105)
5′-GGGUCCACCACTAGCCAGUA-3′;
(106)
5′-GUAGAGAAATTATTTUAGGA-3′;
(107)
5′-AUCCACCACGTCGTCCAUGU-3′;
(108)
5′-GGCAUCCACCACGTCGUCCA-3′;
(109)
5′-UUACUTTAAAAGCAAAAGGA-3′;
(110)
5′-UUUGAAGTGAAGTAAAAGGA-3′;
(111)
5′-GAAGUGAAGTAAAAGGAGAA-3′;
(112)
5′-GGCCATCTCTGCTTCUUGGU-3′;
(113)
5′-UGGGCTGGAATCCGAGUUAU-3′;
(114)
5′-GGAGATTTCAGAGCAGCUUC-3′;
(115)
5′-UUUACGGTTTTCAGAAUAUC-3′;
(116)
5′-GCGUGTCTGGAAGCTUCCUU-3′;
(117)
5′-GCUUATTTTAAGCATAUUAA-3′;
(118)
5′-UUAUUTTAAGCATATUAAAA-3′;
(119)
5′-UUCUGCAGCTTCCTTGUCCU-3′;
(120)
5′-AUUACTTTAAAAGCAAAAGG-3′;
(121)
5′-AUUUUAAGCATATTAAAAAG-3′;
(122)
5′-UGUGGCTTGTCCTCAGACAU-3′;
(123)
5′-AAAAGGAGAAAACTAUCUUC-3′;
(124)
5′-GGGUCCATACCCAAGGCAUC-3′;
(125)
5′-ACAGUGTTGAGATACUCGGG-3′;
(126)
5′-GGAUCTGCATGCCCTCAUCU-3′;
(127)
5′-AGUAAAAAGCTTTTGAAGUG-3′;
(128)
5′-GUCGUGGCAAATAGTCCUAG-3′;
(129)
5′-GGAGATCAGATGAGAGGAGC-3′;
(130)
5′-GUGGUTAAGTACATGAGCUC-3′;
(131)
5′-GGACACTTAGCTGTTCCUCG-3′;
(132)
5′-GUCUCTACTGTTACCUCUGA-3′;
(133)
5′-GAGUUCTTCGTAGGCUUCUG-3′;
(134)
5′-AAAGUCAAAAAGAAAAACUG-3′;
(135)
5′-AAAAGTGGGAAATAAAGGUU-3′;
(136)
5′-AGUUUATAGATTTCAAGUAG-3′;
(137)
5′-AAAAAGTGGGAAATAAAGGU-3′;
(138)
5′-UUUAUATTACAAAGCUACUU-3′;
(139)
5′-UGCUATTCATATTTTUAUUU-3′;
(140)
5′-GGUAU-3′;
(141)
5′-[G/A/C][G/A][G/A/C][T/A/C]TC-3′;
(142)
5′-[G/A/C]G[G/A/C][T/A/C]TC-3′;
(143)
5′-GGCTTC-3′;
(144)
5′-GGCATC-3′;
(145)
5′-AGCTTC-3′;
(146)
5′-GGAATC-3′;
(147)
5′-CACATC-3′;
(148)
5′-GGCCTC-3′;
(149)
5′-CACTTC-3′;
(150)
5′-AAGATC-3′;
(151)
5′-TGTCCTTGCACGTGGCTTCG-3′;
(152)
5′-TTTGCACACTTCGTACCCAA-3′;
(153)
5′-GTCCACATCCTGTGGCTCGT-3′;
(154)
5′-TGTGATGGCCTCCCATCTCC-3′;
(155)
5′-GGTTTTGGCTGGGATCAAGT-3′;
(156)
5′-GGTGTCCTTGCACGTGGCTT-3′;
(157)
5′-GGTCCATACCCAAGGCATCC-3′;
(158)
5′-GTGTCTTCATCGGCCCTGCC-3′;
(159)
5′-GTCTTGGCTTCGTGGAGCAG-3′;
(160)
5′-GCTGACAAAGATTCACTGGT-3′;
(161)
5′-GAAAGGTTATGCAAGG-3′;
(162)
5′-GACTATACGCGCAATA-3′;
(163)
5′-TGTGATGGCCTCCCAT-3′;
(164)
5′-TCCAACACTTCGTGGG-3′;
(165)
5′-GTGTCTGGAAGCTTCC-3′;
(166)
5′-CTTGAAGCATCGTATC-3′;
(167)
5′-TCGTAGTTGCTTCCTA-3′;
(168)
5′-CGCTTTTCTGTCTGGT-3′;
(169)
5′-GGCTGGAATCCGAGTT-3′;
(170)
5′-GATAGCACCTTCAGCA-3′;
(171)
5′-AGGACTCCAGATGTTT-3′;
(172)
5′-GTGATCTTGACATGCT-3′;
(173)
5′-AGATTTCAGAGCAGCT-3′;
(174)
5′-GGTTACGGCTCAGTAT-3′;
(175)
5′-GTTCAGTCCTGTCCAT-3′;
(176)
5′-AGGTCTTGGCTTCGTG-3′;
(177)
5′-CTGCAGCTTCCTTGTC-3′;
(178)
5′-GTCCTTGCACGTGGCT-3′;
(179)
5′-GTCTCTGGAGCTTCCT-3′;
(180)
5′-GGTCTTGGCTTCGTGG-3′;
(181)
5′-GGUA-3′;
(182)
5′-GUAU-3′;
(183)
5′-GGU-3′;
(184)
5′-GUA-3′;
(185)
5′-GUC-3′;
(186)
5′-GUG-3′;
(187)
5′-GUU-3′;
(188)
5′-GUA-3′;
(189)
5′-GGC-3′;
(190)
5′-AUC-3′;
(191)
5′-GAA-3′;
(192)
5′-GAG-3′;
(193)
5′-GGA-3′;
(194)
5′-GAC-3′;
(195)
5′-GAU-3′;
(196)
5′-AUG-3′;
(197)
5′-GCG-3′;
(198)
5′-UUC-3′;
(199)
5′-GCC-3′;
(200)
5′-GGG-3′;
(201)
5′-AUU-3′;
(202)
5′-GCA-3′;
(203)
5′-AGC-3′;
(204)
5′-AAC-3′;
(205)
5′-CCA-3′;
(206)
5′-UGC-3′;
(207)
5′-CAA-3′;
(208)
5′-CGG-3′;
(209)
5′-ACC-3′;
(210)
5′-AGA-3′;
(211)
5′-TTT-3′;
(212)
5′-TCT-3
and
and
(213) a variant of the motifs or sequences of (1) to (212) having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR7 activity, and
iv) selecting an oligonucleotide which inhibits TLR7 activity.

49. A method for increasing the TLR7 inhibitory activity of an oligonucleotide, the method comprising modifying the oligonucleotide such that the modified oligonucleotide comprises a motif including a sequence selected from the group consisting of:
(1)
5′-[A/G]GU[A/C][U/C]C-3′;
(2)
5′-A[G/A][U/G]C[U/C]C-3′;
(3)
5′-A[G/A][U/G]C[U/C]C[U/C][C/A]U-3′;
(4)
5′-GGUAUA-3′;
(5)
5′-UGUUUC-3′;
(6)
5′-UGUGUC-3′;
(7)
5′-CGUGUC-3′;
(8)
5′-GCAGUCTCCATGTCCCAGGC-3′;
(9)
5′-GAUGGTTCCAGTCCCUCUUC-3′;
(10)
5′-AGCAGTCTCCATGTCCCAGG-3′;
(11)
5′-GGGUCTCCTCCACACCCUUC-3′;
(12)
5′-GGUGGCCACAGGCAACGUCA-3′;
(13)
5′-GCCGUTTCCATGTCCCAGGC-3′;
(14)
5′-GCGGUATCCATGTCCCAGGC-3′;
(15)
5′-GCGGUATACAGGTCCCAGGC-3′;
(16)
5′-GCUGUTTCCATGTCCCAGGC-3′;
(17)
5′-GCUGUGTCCATGTCCCAGGC-3′;
(18)
5′-GCCGUGTCCATGTCCCAGGC-3′;
(19)
5′-GCGGUAUCCAUGUCCCAGGC-3′;
(20)
5′-GGUATCCCCCCCCCCCCCCC-3′;
(21)
5′-GUCCCATCCCTTCTGCUGCC-3′;
(22)
5′-UUCUCTCTGGTCCCAUCCCU-3′;
(23)
5′-GUUCAGTCAGATCGCUGGGA-3′;
(24)
5′-AUGACATTTCGTGGCUCCUA-3′;
(25)
5′-UCUCCATGTCCCAGGCCUCC-3′;
(26)
5′-AGUCUCCATGTCCCAGGCCU-3′;
(27)
5′-CCAUGTCCCAGGCCTCCAGU-3′;
(28)
5′-GCAAGGCAGAGAAACUCCAG-3′;
(29)
5′-GGAUUAAAACAGATTAAUAC-3′;
(30)
5′-AGCCGAACAGAAGGAGCGUC-3′;
(31)
5′-UCCGGCCTCGGAAGCUCUCU-3′;
(32)
5′-UCCGGCCTCGGAGTCUCCAU-3′;
(33)
5′-GCGGUATCCATAGTCUCCAU-3′;
(34)
5′-GAUUAAAACAGATTAAUACA-3′;
(35)
5′-UGACAAAACAATAATAACAG-3′;
(36)
5′-CCAACACTTCGTGGGGUCCU-3′;
(37)
5′-GUGUCCTTCATGCTTUGGAU-3′;
(38)
5′-GUCCCAGGCCTCCAGUGUCU-3′;
(39)
5′-UUGGCCTGTGGATGCUUUGU-3′;
(40)
5′-GUCCGTACCTCCACCCACCG-3′;
(41)
5′-GUGUUTTTAATTTTGUAGAG-3′;
(42)
5′-GUCAAACCTAGAAAGAAGCA-3′;
(43)
5′-GGUCUCCTCCACACCCUUCU-3′;
(44)
5′-GUCUCCTCCACACCCUUCUC-3′;
(45)
5′-UGAUGATGCTTGCAGGAGGC-3′;
(46)
5′-UUAAATAATCTAGTTUGAAG-3′;
(47)
5′-AAAGCAGTCTCCATGUCCCA-3′;
(48)
5′-GCGUAGTTTCTCTTCCUCCC-3′;
(49)
5′-UCUGGTCCCATCCCTUCUGC-3′;
(50)
5′-UAUUUCCACATGCCCAGUGU-3′;
(51)
5′-UGUUUCCCCGGAGAGCAAUG-3′;
(52)
5′-UUAGCTCCTTGCCTCGUUCC-3′;
(53)
5′-AUUUCCACATGCCCAGUGUU-3′;
(54)
5′-UGGCGTAGTTTCTCTUCCUC-3′;
(55)
5′-UGACATTTCGTGGCTCCUAC-3′;
(56)
5′-GAAAAGATTATCTTCUUUUA-3′;
(57)
5′-UGUGAAAAGATTATCUUCUU-3′;
(58)
5′-UUGUGAAAAGATTATCUUCU-3′;
(59)
5′-UUUGAAATTCAGAAGAUUUG-3′;
(60)
5′-AAGCAGTCTCCATGTCCCAG-3′;
(61)
5′-AGGAUTAAAACAGATUAAUA-3′;
(62)
5′-AGAUUATCTTCTTTTAAUUU-3′;
(63)
5′-UCCCAACTCTTCTAACUCGU-3′;
(64)
5′-UAAAATAAGGGGAATAGGGG-3′;
(65)
5′-AGUGGCACATACCACACCCU-3′;
(66)
5′-AAGAUTATCTTCTTTUAAUU-3′;
(67)
5′-AGAAAGAAGCAAAGAUUCAA-3′;
(68)
5′-UCCCATCCCTTCTGCUGCCA-3′;
(69)
5′-ACCCUTCTCTCTGGTCCCAU-3′;
(70)
5′-AAUAUCTGCTGCCCACCUUC-3′;
(71)
5′-UCUCUCTGGTCCCATCCCUU-3′;
(72)
5′-AGGCCTCCAGTGTCTUCUCC-3′;
(73)
5′-CAAGCCCCAGCGTTCCUCCG-3′;
(74)
5′-AAAUGTCCTGGCCCTCACUG-3′;
(75)
5′-AAUUUAAAGCATGAAUAUUA-3′;
(76)
5′-AAAAGATTATCTTCTUUUAA-3′;
(77)
5′-AUAUCTGCTGCCCACCUUCU-3′;
(78)
5′-CAGUCTCCATGTCCCAGGCC-3′;
(79)
5′-AAAGATTATCTTCTTUUAAU-3′;
(80)
5′-CCCUUCTCTCTGGTCCCAUC-3′;
(81)
5′-AUGGCCTTTCCGTGCCAAGG-3′;
(82)
5′-GCAUUCCGTGCGGAAGCCUU-3′;
(83)
5′-GGCCGAACTTTCCCGCCUUA-3′;
(84)
5′-GGUCUTGGCTTCGTGGAGCA-3′;
(85)
5′-GGAGCTTCGAGGCCCCAGGC-3′;
(86)
5′-GGUGGTCCACAACCCCUUUC-3′;
(87)
5′-UUCUGGGGACTTCCAGUUUA-3′;
(88)
5′-UGAUUCCAAAGCCAGGGUUA-3′;
(89)
5′-GGGUATCGAAAGAGTCUGGA-3′;
(90)
5′-CUUGCACGTGGCTTCGUCUC-3′;
(91)
5′-GUGUCCTTGCACGTGGCUUC-3′;
(92)
5′-GUAAAAAGCTTTTGAAGUGA-3′;
(93)
5′-AUGCCATCCACTTGAUAGGC-3′;
(94)
5′-UGAAGTAAAAATCAAUAGCG-3′;
(95)
5′-AAGGCCCTTCGCACTUCUUA-3′;
(96)
5′-GUACUCGTCGGCATCCACCA-3′;
(97)
5′-GUCCUTGCACGTGGCUUCGU-3′;
(98)
5′-GCCCATCCATGAGGTCCUGG-3′;
(99)
5′-GUAAAAGGAGAAAACUAUCU-3′;
(100)
5′-UUGAAGTGAAGTAAAAGGAG-3′;
(101)
5′-GUUACTCGTGCCTTGGCAAA-3′;
(102)
5′-GUCCAAGATCAGCAGUCUCA-3′;
(103)
5′-UUCAATGGGAGAATAAAGCA-3′;
(104)
5′-GCAAGGCCCTTCGCACUUCU-3′;
(105)
5′-GGGUCCACCACTAGCCAGUA-3′;
(106)
5′-GUAGAGAAATTATTTUAGGA-3′;
(107)
5′-AUCCACCACGTCGTCCAUGU-3′;
(108)
5′-GGCAUCCACCACGTCGUCCA-3′;
(109)
5′-UUACUTTAAAAGCAAAAGGA-3′;
(110)
5′-UUUGAAGTGAAGTAAAAGGA-3′;
(111)
5′-GAAGUGAAGTAAAAGGAGAA-3′;
(112)
5′-GGCCATCTCTGCTTCUUGGU-3′;
(113)
5′-UGGGCTGGAATCCGAGUUAU-3′;
(114)
5′-GGAGATTTCAGAGCAGCUUC-3′;
(115)
5′-UUUACGGTTTTCAGAAUAUC-3′;
(116)
5′-GCGUGTCTGGAAGCTUCCUU-3′;
(117)
5′-GCUUATTTTAAGCATAUUAA-3′;
(118)
5′-UUAUUTTAAGCATATUAAAA-3′;
(119)
5′-UUCUGCAGCTTCCTTGUCCU-3′;
(120)
5′-AUUACTTTAAAAGCAAAAGG-3′;
(121)
5′-AUUUUAAGCATATTAAAAAG-3′;
(122)
5′-UGUGGCTTGTCCTCAGACAU-3′;
(123)
5′-AAAAGGAGAAAACTAUCUUC-3′;
(124)
5′-GGGUCCATACCCAAGGCAUC-3′;
(125)
5′-ACAGUGTTGAGATACUCGGG-3′;
(126)
5′-GGAUCTGCATGCCCTCAUCU-3′;
(127)
5′-AGUAAAAAGCTTTTGAAGUG-3′;
(128)
5′-GUCGUGGCAAATAGTCCUAG-3′;
(129)
5′-GGAGATCAGATGAGAGGAGC-3′;
(130)
5′-GUGGUTAAGTACATGAGCUC-3′;
(131)
5′-GGACACTTAGCTGTTCCUCG-3′;
(132)
5′-GUCUCTACTGTTACCUCUGA-3′;
(133)
5′-GAGUUCTTCGTAGGCUUCUG-3′;
(134)
5′-AAAGUCAAAAAGAAAAACUG-3′;
(135)
5′-AAAAGTGGGAAATAAAGGUU-3′;
(136)
5′-AGUUUATAGATTTCAAGUAG-3′;
(137)
5′-AAAAAGTGGGAAATAAAGGU-3′;
(138)
5′-UUUAUATTACAAAGCUACUU-3′;
(139)
5′-UGCUATTCATATTTTUAUUU-3′;
(140)
5′-GGUAU-3′;
(141)
5′-[G/A/C][G/A][G/A/C][T/A/C]TC-3′;
(142)
5′-[G/A/C]G[G/A/C][T/A/C]TC-3′;
(143)
5′-GGCTTC-3′;
(144)
5′-GGCATC-3′;
(145)
5′-AGCTTC-3′;
(146)
5′-GGAATC-3′;
(147)
5′-CACATC-3′;
(148)
5′-GGCCTC-3′;
(149)
5′-CACTTC-3′;
(150)
5′-AAGATC-3′;
(151)
5′-TGTCCTTGCACGTGGCTTCG-3′;
(152)
5′-TTTGCACACTTCGTACCCAA-3′;
(153)
5′-GTCCACATCCTGTGGCTCGT-3′;
(154)
5′-TGTGATGGCCTCCCATCTCC-3′;
(155)
5′-GGTTTTGGCTGGGATCAAGT-3′;
(156)
5′-GGTGTCCTTGCACGTGGCTT-3′;
(157)
5′-GGTCCATACCCAAGGCATCC-3′;
(158)
5′-GTGTCTTCATCGGCCCTGCC-3′;
(159)
5′-GTCTTGGCTTCGTGGAGCAG-3′;
(160)
5′-GCTGACAAAGATTCACTGGT-3′;
(161)
5′-GAAAGGTTATGCAAGG-3′;
(162)
5′-GACTATACGCGCAATA-3′;
(163)
5′-TGTGATGGCCTCCCAT-3′;
(164)
5′-TCCAACACTTCGTGGG-3′;
(165)
5′-GTGTCTGGAAGCTTCC-3′;
(166)
5′-CTTGAAGCATCGTATC-3′;
(167)
5′-TCGTAGTTGCTTCCTA-3′;
(168)
5′-CGCTTTTCTGTCTGGT-3′;
(169)
5′-GGCTGGAATCCGAGTT-3′;
(170)
5′-GATAGCACCTTCAGCA-3′;
(171)
5′-AGGACTCCAGATGTTT-3′;
(172)
5′-GTGATCTTGACATGCT-3′;
(173)
5′-AGATTTCAGAGCAGCT-3′;
(174)
5′-GGTTACGGCTCAGTAT-3′;
(175)
5′-GTTCAGTCCTGTCCAT-3′;
(176)
5′-AGGTCTTGGCTTCGTG-3′;
(177)
5′-CTGCAGCTTCCTTGTC-3′;
(178)
5′-GTCCTTGCACGTGGCT-3′;
(179)
5′-GTCTCTGGAGCTTCCT-3′;
(180)
5′-GGTCTTGGCTTCGTGG-3′;
(181)
5′-GGUA-3′;
(182)
5′-GUAU-3′;
(183)
5′-GGU-3′;
(184)
5′-GUA-3′;
(185)
5′-GUC-3′;
(186)
5′-GUG-3′;
(187)
5′-GUU-3′;
(188)
5′-GUA-3′;
(189)
5′-GGC-3′;
(190)
5′-AUC-3′;
(191)
5′-GAA-3′;
(192)
5′-GAG-3′;
(193)
5′-GGA-3′;
(194)
5′-GAC-3′;
(195)
5′-GAU-3′;
(196)
5′-AUG-3′;
(197)
5′-GCG-3′;
(198)
5′-UUC-3′;
(199)
5′-GCC-3′;
(200)
5′-GGG-3′;
(201)
5′-AUU-3′;
(202)
5′-GCA-3′;
(203)
5′-AGC-3′;
(204)
5′-AAC-3′;
(205)
5′-CCA-3′;
(206)
5′-UGC-3′;
(207)
5′-CAA-3′;
(208)
5′-CGG-3′;
(209)
5′-ACC-3′;
(210)
5′-AGA-3′;
(211)
5′-TTT-3′;
(212)
5′-TCT-3
and
and
(213) a variant of the motifs or sequences of (1) to (212) having at least about 75% sequence identity thereto;
wherein the U may be a T and/or the T may be a U.

50. The method of claim 49, wherein the step of modifying the oligonucleotide includes adding a sequence of nucleotides to the 5′ and/or 3′ end of the oligonucleotide such that the modified oligonucleotide comprises the motif.

51. The method of claim 49 or 50, which further comprises testing the ability of the modified oligonucleotide to inhibit TLR7 activity, and selecting an oligonucleotide which inhibits TLR7 activity to a greater extent than the unmodified oligonucleotide.

52. The method according to any one of claims 48 to 51, wherein the oligonucleotide does not bind or is not designed to bind a transcript that encodes TLR7 or a complement thereof.

53. The method according to any one of claims 48 to 52, wherein the oligonucleotide binds or is designed to bind a target transcript that does not encode TLR7 or a complement thereof.

54. The method according to any one of claims 48 to 53, wherein the motif is: (a) within eleven bases of the 5′ and/or 3′ end of the oligonucleotide; (b) within eight bases of the 5′ and/or 3′ end of the oligonucleotide; and/or (c) at or towards the 5′ and/or 3′ end of the oligonucleotide.

55. The method according to any one of claims 48 to 54, wherein the oligonucleotide comprises
a) a 5′ region comprising bases which are modified and/or which have a modified backbone,
b) a middle region comprising ribonucleic acid, deoxyribonucleic acid, or combination thereof, bases, which optionally have a modified backbone, and
c) a 3′ region comprising bases which are modified and/or which have a modified backbone.

56. The method of claim 55, wherein the middle region is about 10 bases in length and/or the 5′ region and/or the 3′ region are: (a) about 3 bases in length; or (b) about 5 bases in length.

57. The method according to any one of claims 48 to 56, wherein the motif has the sequence 5′-GGUAUC-3′, 5′-AGUCUC-3′, 5′-GGUCCC-3′, 5′-GGUCUC-3′, 5′-AAGCUC-3′, 5′-AGUCCC-3′, 5′-GGUAUA-3′, 5′-UGUUUC-3′, 5′-UGUGUC-3′, 5′-CGUUUC-3′, 5′-CGUGUC-3′, 5′-GGUAU-3′, 5′-GGUA-3′, 5′-GUAU-3′, 5′-GGU-3′, 5′-GUA-3′, (185) 5′-GUC-3′; 5′-GUG-3′; 5′-GUU-3′; 5′-GUA-3′; 5′-GGC-3′; 5′-AUC-3′; 5′-GAA-3′; 5′-GAG-3′; 5′-GGA-3′; 5′-GAC-3′; 5′-GAU-3′; 5′-AUG-3′; 5′-GCG-3′; 5′-UUC-3′; 5′-GCC-3′; 5′-GGG-3′; 5′-AUU-3′; 5′-GCA-3′; 5′-AGC-3′; 5′-AAC-3′; 5′-CCA-3′; 5′-UGC-3′; 5′-CAA-3′; 5′-CGG-3′; 5′-ACC-3′; 5′-AGA-3′, wherein the U may be a T.

58. The method according to any one of claims 48 to 57, wherein the motif has the sequence of 5′-GGUAUC-3′, 5′-GGUATC-3′, 5′-AGUCTC-3′, 5′-AGTCTC-3′, 5′-GGUCCC-3′, 5′-GGUCTC-3′, 5′-AAGCUC-3′, 5′-AGTCCC-3′, 5′-GGUATA-3′, 5′-UGUTTC-3′, 5′-UGUGTC-3′, 5′-CGUTTC-3′, 5′-CGUGTC-3′, 5′-GGUAU-3′, 5′-GGUAT-3′, 5′-GGUA-3′, 5′-GUAU-3′, 5′-GUAT-3′, 5′-GGU-3′ or 5′-GUA-3′.

59. The method according to any one of claims 48 to 58, wherein one or more of the bases of the motif are a modified base and/or have a modified backbone.

60. The method according to any one of claims 48 to 59, wherein the motif has the sequence of 5′-mGmGmUATC-3′, 5′-mAmGmUCTC-3′, 5′-mAmGTCTC-3′, 5′-mGmGmUmCmCC-3′, 5′-mGmGmUmCTC-3′, 5′-AAGCmUmC-3′, 5′-AGTCCC-3′, 5′-mGmGmUATA-3′, 5′-mUmGmUTTC-3′, 5′-mUmGmUGTC-3′, 5′-mCmGmUTTC-3′, 5′-mCmGmUGTC-3′, 5′-mGmGmUAU-3′, 5′-mGmGmUAT-3′, 5′-mGmGmUmAU-3′, 5′-mGmGmUmAT-3′, 5′-mGmGmUmAmU-3′, 5′-mGmGmUmA-3′, 5′-mGmUmAmU-3′, 5′-mGmGmU-3′ or 5′-mGmUmA-3′, wherein m is a modified base and/or has a modified backbone.

61. A method for selecting or designing an oligonucleotide which increases or potentiates TLR8 activity, the method comprising
i) scanning a polynucleotide, or complement thereof, for a region having a motif including a sequence selected from the group consisting of:
5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′;
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;
and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to increase or potentiate TLR8 activity, and
iv) selecting an oligonucleotide which increases or potentiate TLR8 activity.

62. A method for increasing or potentiating the TLR8 activity of an oligonucleotide, or increasing the potentiating activity of an oligonucleotide, the method comprising modifying the oligonucleotide such that the modified oligonucleotide comprises a motif including a sequence selected from the group consisting of:
5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′:
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;
and
a variant of the motifs or sequences thereof having at least about 75% sequence identity thereto;
wherein the U may be a T and/or the T may be a U.

63. A method for selecting or designing an oligonucleotide which inhibits TLR8 activity, the method comprising
i) scanning a polynucleotide, or complement thereof, for a region having a motif including a sequence selected from the group consisting of:
5′-GAG-3′;
5′-GAC-3′:
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5-GCU-3′;
5′-CCU-3′;
5′-CUA-3′
5′-CUC-3′;
5′-AAC-3′:
and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR8 activity, and
iv) selecting an oligonucleotide which inhibits TLR8 activity.

64. A method for increasing the TLR8 inhibitory activity of an oligonucleotide, the method comprising modifying the oligonucleotide such that the modified oligonucleotide comprises a motif including a sequence selected from the group consisting of:
5′-GAG-3′;
5′-GAC-3′;
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5-GCU-3′;
5′-CCU-3′;
5′-CUA-3′;
5′-CUC-3′;
5′-AAC-3′:
and
a variant of the motifs or sequences thereof having at least about 75% sequence identity thereto;
wherein the U may be a T and/or the T may be a U.

65. A method for selecting or designing an oligonucleotide which inhibits TLR3 activity, the method comprising
i) scanning a polynucleotide, or complement thereof, for a region having a motif including a sequence selected from the group consisting of:
5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′;
5′-TAG-3′;
5′-TGG-3′;
5′-TCA-3′;
5′-TGC-3′;
5′-CAC-3′;
5′-CGG-3′;
5′-CCC-3′;
5′-ACT-3′;
5′-GTA-3′;
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;
and
a variant of the motifs or sequences thereof having at least about 75% sequence identity thereto;
wherein the U may be a T and/or the T may be a U;
ii) producing one or more candidate oligonucleotides comprising the motif,
iii) testing the ability of the one or more candidate oligonucleotides to inhibit TLR3 activity, and
iv) selecting an oligonucleotide which inhibits TLR3 activity.

66. A method for increasing the TLR3 inhibitory activity of an oligonucleotide, the method comprising modifying the oligonucleotide such that the modified oligonucleotide comprises a motif including a sequence selected from the group consisting of:
5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′;
5′-TAG-3′;
5′-TGG-3′;
5′-TCA-3′;
5′-TGC-3′;
5′-CAC-3′;
5′-CGG-3′;
5′-CCC-3′;
5′-ACT-3′;
5′-GTA-3′;
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;
and
a variant of the motifs or sequences thereof having at least about 75% sequence identity thereto;
wherein the U may be a T and/or the T may be a U.

67. The method according to any one of claims 12, 13, 22, 23, 36 to 47, 59 or 60, wherein the modified base comprises a 2′-O-methyl, 2′-O-methoxyethoxy, 2′-fluoro, 2′-allyl, 2′-O-[2-(methylamino)-2-oxoethyl], 4′-thio, 4′-CH2-O-2′-bridge, 4′-(CH2)2-O-2′-bridge, 2′-LNA, 2′-amino, fluoroarabinonucleotide, threose nucleic acid or 2′-O-(N-methlycarbamate).

68. The method according to any one of claims 12, 13, 22, 23, 36 to 47, 59 or 60, wherein the modified backbone comprises a phosphorothioate, a non-bridging oxygen atom substituting a sulfur atom, a phosphonate such as a methylphosphonate, a phosphodiester, a phosphoromorpholidate, a phosphoropiperazidate, amides, methylene(methylamino), fromacetal, thioformacetal, a peptide nucleic acid or a phosphoroamidate such as a morpholino phosphorodiamidate (PMO), N3′-P5′ phosphoramidite or thiophosphoroamidite.

69. The method according to any one of the preceding claims, wherein at least a portion of the oligonucleotide has/is a ribonucleic acid, deoxyribonucleic acid, DNA phosphorothioate, RNA phosphorothioate, 2′-O-methyl-oligonucleotide, 2′-O-methyl-oligodeoxyribonucleotide, 2′-O-hydrocarbyl ribonucleic acid, 2′-O-hydrocarbyl DNA, 2′-O-hydrocarbyl RNA phosphorothioate, 2′-O-hydrocarbyl DNA phosphorothioate, 2′-F-phosphorothioate, 2′-F-phosphodiester, 2′-methoxyethyl phosphorothioate, 2-methoxyethyl phosphodiester, deoxy methylene(methylimino) (deoxy MMI), 2′-O-hydrocarby MMI, deoxy-methylphos-phonate, 2′-O-hydrocarbyl methylphosphonate, morpholino, 4′-thio DNA, 4′-thio RNA, peptide nucleic acid, 3′-amidate, deoxy 3′-amidate, 2′-O-hydrocarbyl 3′-amidate, locked nucleic acid, cyclohexane nucleic acid, tricycle-DNA, 2′fluoro-arabino nucleic acid, N3′-P5′ phosphoroamidate, carbamate linked, phosphotriester linked, a nylon backbone modification and any combination thereof

70. The method according to any one of claims 12, 13, 22, 23, 36 to 47, 59 or 60, wherein the modified base comprises:
(a) a 2′O-methyl and a phosphorothioate backbone;
(b) a 2′-LNA and a phosphorothioate backbone; or
(c) a 2′-O-methoxyethoxy and a phosphorothioate backbone.

71. The method according to any one of the preceding claims, wherein at least one of the bases of the oligonucleotide does not hybridize to a target polynucleotide.

72. The method according to any one of the preceding claims, wherein the oligonucleotide is an antisense oligonucleotide or a double stranded oligonucleotide for gene silencing.

73. The method of claim 72, wherein the oligonucleotide is a gapmer antisense oligonucleotide.

74. The method of claim 72 or claim 73, wherein one or more bases of the motif or the oligonucleotide are removed by an endonuclease in vivo.

75. The method of claim 72, wherein the double stranded oligonucleotide for gene silencing is an siRNA or an shRNA.

76. An oligonucleotide selected or designed using the method according to any one of claims 1, 5 to 14, 18 to 24, 28 to 48, or 52 to 75 or modified using the method according to any one of claims 2 to 13, 15 to 23, 25 to 41 or 49 to 75.

77. An oligonucleotide comprising a motif or sequence selected from the group consisting of:
(1)
5′-[A/G]GU[A/C][U/C]C-3′;
(2)
5′-A[G/A][U/G]C[U/C]C-3′;
(3)
5′-A[G/A][U/G][U/C]C[U/C][C/A]U-3′;
(4)
5′-GGUAUA-3′;
(5)
5′-UGUUUC-3′;
(6)
5′-UGUGUC-3′;
(7)
5′-CGUUUC-3′;
(8)
5′-CGUGUC-3′;
(9)
5′-AUGGCCTTTCCGTGCCAAGG-3′;
(10)
5′-UCCGGCCTCGGAAGCUCUCU-3′;
(11)
5′-GCAUUCCGTGCGGAAGCCUU-3′;
(12)
5′-GGCCGAACTTTCCCGCCUUA-3′;
(13)
5′-GGUCUTGGCTTCGTGGAGCA-3′;
(14)
5′-GGAGCTTCGAGGCCCCAGGC-3′;
(15)
5′-GGUGGTCCACAACCCCUUUC-3′;
(16)
5′-CAUUAGGTGCAGAAAUCUUC-3′;
(17)
5′-UUCUGGGGACTTCCAGUUUA-3′;
(18)
5′-UGAUUCCAAAGCCAGGGUUA-3′;
(19)
5′-CUUUAGTCGTAGTTGCUUCC-3′;
(20)
5′-UUAAATAATCTAGTTUGAAG-3′;
(21)
5′-GUGUCCTTCATGCTTUGGAU-3′;
(22)
5′-AGAAAGAAGCAAAGAUUCAA-3′;
(23)
5′-AGAUUATCTTCTTTTAAUUU-3′;
(24)
5′-AAAAGATTATCTTCTUUUAA-3′;
(25)
5′-GAAAAGATTATCTTCUUUUA-3′;
(26)
5′-UGUGAAAAGATTATCUUCUU-3′;
(27)
5′-CUUGUGAAAAGATTAUCUUC-3′;
(28)
5′-GGUGGCCACAGGCAACGUCA-3′;
(29)
5′-CCAUGTCCCAGGCCTCCAGU-3′;
(30)
5′-GCAGUCTCCATGTCCCAGGC-3′;
(31)
5′-AGCAGTCTCCATGTCCCAGG-3′;
(32)
5′-AGUGGCACATACCACACCCU-3′;
(33)
5′-AUUUCCACATGCCCAGUGUU-3′;
(34)
5′-GGUCCCATCCCTTCTGCUGC-3′;
(35)
5′-UCUGGTCCCATCCCTUCUGC-3′;
(36)
5′-GGGUCTCCTCCACACCCUUC-3′;
(37)
5′-GCAAGGCAGAGAAACUCCAG-3′;
(38)
5′-GAUGGTTCCAGTCCCUCUUC-3′;
(39)
5′-UGUUUCCCCGGAGAGCAAUG-3′;
(40)
5′-AGCCGAACAGAAGGAGCGUC-3′;
(41)
5′-GCGUAGTTTCTCTTCCUCCC-3′;
(42)
5′-GCGGUATCCATGTCCCAGGC-3′;
(43)
5′-GCGGUATACAGGTCCCAGGC-3′;
(44)
5′-GCUGUTTCCATGTCCCAGGC-3′;
(45)
5′-GCUGUGTCCATGTCCCAGGC-3′;
(46)
5′-GCCGUTTCCATGTCCCAGGC-3′;
(47)
5′-GCCGUGTCCATGTCCCAGGC-3′;
(48)
5′-GCGGUATCCATAGTCUCCAU-3′;
(49)
5′-GCGGUATCCATCAGAUAUCG-3′;
(50)
5′-CUUUAGTCGTAGTTGUCUCU-3′;
(51)
5′-UCCGGGTCGTAGTTGCUUCC-3′;
(52)
5′-UCCGGCCTCGGAGTCUCCAU-3′;
(53)
5′-UCCGGCCTCGGGAGAUCUCU-3′;
(54)
5′-GGUATCCCCCCCCCCCCCCC-3′;
(55)
5′-GGAUUAAAACAGATTAAUAC-3′;
(56)
5′-GGUAU-3′;
(57)
5′-GGUA-3′;
(58)
5′-GUAU-3′;
(59)
5′-GGU-3′;
(60)
5′-GUA-3′;
(61)
5′-TGTCTG-3′;
(62)
5′-GTCT-3′;
(63)
5′-TCTCCG-3′;
(64)
5′-CTCC-3′;
(65)
5′-[G/A][A/C]AG[G/C][T/C]T[C/A];
(66)
5′-AAAGGTTA-3′;
(67)
5′-GAAGCTTC-3′;
(68)
5′-GCAGGCTC-3′;
(69)
5′-A[G/A]GGTT-3′;
(70)
5′-AGGGTT-3′;
(71)
5′-AAGGTT-3′;
(72)
5′-GGTT-3′;
(73)
5′-[A/G]GCT[T/C][T/C][G/C][T/A]-3′;
(74)
5′-AGCTTCCT-3′;
(75)
5′-AGCTTCGA-3′;
(76)
5′-GGCTTCGT-3′;
(77)
5′-TGCTTCCT-3′;
(78)
5′-AGCTCTCT-3′;
(79)
5′-G[G/C]TT-3′;
(80)
5′-GCTT-3′;
(81)
5′-CGGAGGTCTTGGCTTCGTGG-3′;
(82)
5′-AGGTCTTGGCTTCGTGGAGC-3′;
(83)
5′-GGGAAAGGTTATGCAAGGTC-3′;
(84)
5′-CTGTGATCTTGACATGCTGC-3′;
(85)
5′-ACTGACTGTCTTGAGGGTTC-3′;
(86)
5′-GCGTGTCTGGAAGCTTCCTT-3′;
(87)
5′-GAGTCTCTGGAGCTTCCTCT-3′;
(88)
5′-AGTCGTAGTTGCTTCCTAAC-3′;
(89)
5′-GTCTTGGCTTCGTGGAGCAG-3′;
(90)
5′-TTGGCTCGGCTTGCCTACTT-3′;
(91)
5′-ACAGTGTTGAGATACTCGGG-3′;
(92)
5′-TCGCACTTCAGTCTGAGCAG-3′;
(93)
5′-GGTGTCCTTGCACGTGGCTT-3′;
(94)
5′-TTTGCACACTTCGTACCCAA-3′;
(95)
5′-GCTGACAAAGATTCACTGGT-3′;
(96)
5′-GCGGAGGTCTTGGCTTCGTG-3′;
(97)
5′-CCAAGATCAGCAGTCT-3′;
(98)
5′-CTTGAAGCATCGTATC-3′;
(99)
5′-GCACACTTCGTACCCA-3′;
(100)
5′-GATAGCACCTTCAGCA-3′;
(101)
5′-CGTATTATAGCCGATT-3′;
(102)
5′-GCAGGCTCAGTGATGT-3′;
(103)
5′-GAAAGGTTATGCAAGG-3′;
(104)
5′-ATGGCCTCCCATCTCC-3′;
(105)
5′-CGCTTTTCTGTCTGGT-3′;
(106)
5′-GTGTCTGGAAGCTTCC-3′;
(107)
5′-TGGCCTCCCATCTCCT-3′;
(108)
5′-ATCTGGCAGCCCATCA-3′;
(109)
5′-GAGGTCTTGGCTTCGT-3′;
(110)
5′-ACACTTCGTGGGGTCC-3′;
(111)
5′-TTCGTGGGGTCCTTTT-3′;
(112)
5′-CCACTTGGCAGACCAT-3′;
(113)
5′-CCATCCATGAGGTCCT-3′;
(114)
5′-TCCAACACTTCGTGGG-3′;
(115)
5′-TCTTCATCGGCCCTGC-3′;
(116)
5′-CCAGCAGGTCAGCAAA-3′;
(117)
5′-CGCTTTTCTCTCCGGT-3′;
(118)
5′-GGUAUAGGACTCCAGATGUUUCC-3′;
(119) a variant of the motifs or sequences of (1) to (118) having at least about 75% sequence identity thereto;
wherein the U may be a T and/or the T may be a U and wherein the oligonucleotide inhibits cGAS activity when administered to a subject.

78. An oligonucleotide comprising a motif or sequence selected from the group consisting of:
(1)
5′[C/U]CUUCU-3′;
(2)
5′-CACCCTTCTCTCTGGUCCCA-3′;
(3)
5′-CCUUCTCTCTGGTCCCAUCC-3′;
(4)
5′-UCUCUGGTCCCATCCCUUCU-3′;
(5)
5′-AUAUCTGCTGCCCACCUUCU-3′;
(6)
5′-GUCCCATCCCTTCTGCUGCC-3′;
(7)
5′-GUCUCCTCCACACCCUUCUC-3′;
(8)
5′-CAGGCCTCCAGTGTCUUCUC-3′;
(9)
5′-UCCCAACTCTTCTAACUCGU-3′;
(10) a variant of the motifs or sequences of (1) to (9) having at least about 75% sequence identity thereto;
wherein the U may be a T and/or the T may be a U and wherein the oligonucleotide does not inhibit or exhibits reduced inhibition of cyclic-GMP-AMP synthase (cGAS) activity when administered to a subject.

79. An oligonucleotide comprising, consisting essentially of or consisting of a motif or sequence selected from the group consisting of:
(1
5′-G[G/C]CCT[C/G]-3′;
(2)
5′-CUU-3′, wherein the motif is within 10 bases
of the 5′ and/or 3′ end of the oligonucleotide;
(3)
5′-CUUGUGAAAAGATTAUCUUC-3′;
(4)
5′-CUUCUCTCTGGTCCCAUCCC-3′;
(5)
5′-CCUUCTCTCTGGTCCCAUCC-3′;
(6)
5′-CCCUUCTCTCTGGTCCCAUC-3′;
(7)
5′-ACCCUTCTCTCTGGTCCCAU-3′;
(8)
5′-CUUCCACAATCAAGACAUUC-3′;
(9)
5′-CUUCGTGGGGTCCTTUUCAC-3′;
(10)
5′-CACUUCGTGGGGTCCUUUUC-3′;
(11)
5′-CCAACACTTCGTGGGGUCCU-3′;
(12)
5′-UCCGGCCTCGGAAGCUCUCU-3′;
(13)
5′-CCAUGTCCCAGGCCTCCAGU-3′;
(14)
5′-UUGGCCTGTGGATGCUUUGU-3′;
(15)
5′-AAAUGTCCTGGCCCTCACUG-3′;
(16)
5′-UCCGGCCTCGGAGTCUCCAU-3′;
(17)
5′-UCCGGCCTCGGCAGAUAUCG-3′;
(18)
5′-GGCCGAACTTTCCCGCCUUA-3′;
(19)
5′-GGUCUTGGCTTCGTGGAGCA-3′;
(20)
5′-GGAGCTTCGAGGCCCCAGGC-3′;
(21)
5′-GGUGGTCCACAACCCCUUUC-3′;
(22)
5′-CAUUAGGTGCAGAAAUCUUC-3′;
(23)
5′-UUCUGGGGACTTCCAGUUUA-3′;
(24)
5′-UGAUUCCAAAGCCAGGGUUA-3′;
(25)
5′-UCCGGGTCGTAGTTGCUUCC-3′;
(26)
5′-CCUAGAAAGAAGCAAAGAUU-3′;
(27)
5′-GAUUAAAACAGATTAAUACA-3′;
(28)
5′-GGAUUAAAACAGATTAAUAC-3′;
(29)
5′-AAUUUAAAGCATGAAUAUUA-3′;
(30)
5′-GCGUAGTTTCTCTTCCUCCC-3′;
(31)
5′-UGACAAAACAATAATAACAG-3′;
(32)
5′-ACA-3′, wherein the motif is at or towards
the 5′ end of the oligonucleotide;
(33)
5′-CAC-3′, wherein the motif is at or towards
the 5′ end of the oligonucleotide;
(34)
5′-ACACTTCGTGGGGTCCTTTT-3′;
(35)
5′-GCGTGTCTGGAAGCTTCCTT-3′;
(36)
5′-TCAAAGGACTGAGGAAAGGG-3′;
(37)
5′-ATCCAACACTTCGTGGGGTC-3′;
(38)
5′-GCCCATCCATGAGGTCCTGG-3′;
(39)
5′-GGGTATCGAAAGAGTCTGGA-3′;
(40)
5′-GGTTTTGGCTGGGATCAAGT-3′;
(41)
5′-GCGACTATACGCGCAATATG-3′;
(42)
5′-ACTGACTGTCTTGAGGGTTC-3′;
(43)
5′-AACACTTCGTGGGGTCCTTT-3′;
(44)
5′-GTCCAAGATCAGCAGTCTCA-3′;
(45)
5′-ACAGTGTTGAGATACTCGGG-3′;
(46)
5′-TGGGCTGGAATCCGAGTTAT-3′;
(47)
5′-CGGCATCCACCACGTCGTCC-3′;
(48)
5′-GCGTATTATAGCCGATTAAC-3′;
(49)
5′-GGAGGTCTTGGCTTCGTGGA-3′;
(50)
5′-TGGGTTACGGCTCAGTATGG-3′;
(51)
5′-CCGCCATGTTTCTTCTTGGA-3′;
(52)
5′-AGCTTCGAGGCCCCAG-3′;
(53)
5′-GCCATGTTTCTTCTTG-3′;
(54)
5′-CACTTCGTGGGGTCCT-3′;
(55)
5′-CGGCCTCGGAAGCTCT-3′;
(56)
5′-ACACTTCGTGGGGTCC-3′;
(57)
5′-TGCACACTTCGTACCC-3′;
(58)
5′-CCACATCCTGTGGCTC-3′;
(59)
5′-CTGCAGCTTCCTTGTC-3′;
(60)
5′-ACTTCGTGGGGTCCTT-3′;
(61)
5′-CCCACTTGGCAGACCA-3′;
(62)
5′-GTCCCCTGTTGACTGG-3′;
(63)
5′-ACGTTCAGTCCTGTCC-3′;
(64)
5′-GGTCATTACAATAGCT-3′;
(65)
5′-TCGTGGGGTCCTTTTC-3′;
(66)
5′-GTGTCTGGAAGCTTCC-3′;
(67)
5′-ATGGCCTCCCATCTCC-3′;
(68)
5′-TGCTCCTCGGTCTCCC-3′;
(69)
5′-GCATCCACCACGTCGT-3′;
(70)
5′-CTTCGTGGGGTCCTTT-3′;
(71)
5′-AGGCCCTTCGCACTTC-3′;
(72)
5′-GCGGUATCCATGTCCCAGGC-3′;
(73)
5′-GCUGUTTCCATGTCCCAGGC-3′;
(74)
5′-GCUGUGTCCATGTCCCAGGC-3′
(75)
5′-GCCGUTTCCATGTCCCAGGC-3′;
(76)
5′-GCGGUATCC-3′;
(77)
5′-GCUGUTTCC-3′;
(78)
5′-GCUGUGTCC-3′;
(79)
5′-GCCGUTTCC-3′;
(80)
5′-CUUCGTGGGGTCCTTUUCAC;
(81)
5′-CUUCGTGGG-3′;
(82)
5′-UCG-3′;
(83)
5′-ACG-3′;
(84)
5′-ACC-3′;
(85)
5′-CGC-3′;
(86)
5′-GAU-3′;
(87)
5′-GGG-3′;
(88)
5′-AGC-3′;
(89)
5′-UUC-3′;
(90)
5′-UUG-3′;
(91)
5′-CAC-3′;
and
(92) a variant of the motifs or sequences of (1) to (91) having at least about 75% sequence identity thereto;
wherein the U may be a T and/or the T may be a U and wherein the oligonucleotide inhibits TLR9 activity when administered to a subject.

80. An oligonucleotide which comprises:
a) a 5′ region comprising bases which are modified and/or which have a modified backbone,
b) a middle region comprising ribonucleic acid, deoxyribonucleic acid, or combination thereof, bases, which optionally have a modified backbone, wherein at least about 50% of the bases of the middle region are adenine bases; and
c) a 3′ region comprising bases which are modified and/or which have a modified backbone;
wherein the oligonucleotide inhibits TLR9 activity when administered to a subject.

81. The oligonucleotide of claim 80, comprising a motif or sequence selected from the group consisting of:
(1)
5′-GAUUAAAACAGATTAAUACA-3′;
(2)
5′-GGAUUAAAACAGATTAAUAC-3′;
(3)
5′-CUUGUGAAAAGATTAUCUUC-3′;
(4)
5′-CCUAGAAAGAAGCAAAGAUU-3′;
(5)
5′-AAUUUAAAGCATGAAUAUUA-3′;
(6) a variant of the motifs or sequences of (1) to (5) having at least about 75% sequence identity thereto;
wherein the U may be a T and/or the T may be a U.

82. An oligonucleotide comprising a motif or sequence selected from the group consisting of.
(1)
5′-[A/G]GU[A/C][U/C]C-3′;
(2)
5′-A[G/A][U/G]C[U/C]C-3′;
(3)
5′-A[G/A][U/G][U/C]C[U/C][C/A]U-3′;
(4)
5′-GGUAUA-3′;
(5)
5′-UGUUUC-3′;
(6)
5′-UGUGUC-3′;
(7)
5′-CGUGUC-3′;
(8)
5′-GCAGUCTCCATGTCCCAGGC-3′;
(9)
5′-GAUGGTTCCAGTCCCUCUUC-3′;
(10)
5′-AGCAGTCTCCATGTCCCAGG-3′;
(11)
5′-GGGUCTCCTCCACACCCUUC-3′;
(12)
5′-GGUGGCCACAGGCAACGUCA-3′;
(13)
5′-GCCGUTTCCATGTCCCAGGC-3′;
(14)
5′-GCGGUATCCATGTCCCAGGC-3′;
(15)
5′-GCGGUATACAGGTCCCAGGC-3′;
(16)
5′-GCUGUTTCCATGTCCCAGGC-3′;
(17)
5′-GCUGUGTCCATGTCCCAGGC-3′;
(18)
5′-GCCGUGTCCATGTCCCAGGC-3′;
(19)
5′-GCGGUAUCCAUGUCCCAGGC-3′;
(20)
5′-GGUATCCCCCCCCCCCCCCC-3′;
(21)
5′-GUCCCATCCCTTCTGCUGCC-3′;
(22)
5′-UUCUCTCTGGTCCCAUCCCU-3′;
(23)
5′-GUUCAGTCAGATCGCUGGGA-3′;
(24)
5′-AUGACATTTCGTGGCUCCUA-3′;
(25)
5′-UCUCCATGTCCCAGGCCUCC-3′;
(26)
5′-AGUCUCCATGTCCCAGGCCU-3′;
(27)
5′-CCAUGTCCCAGGCCTCCAGU-3′;
(28)
5′-GCAAGGCAGAGAAACUCCAG-3′;
(29)
5′-GGAUUAAAACAGATTAAUAC-3′;
(30)
5′-AGCCGAACAGAAGGAGCGUC-3′;
(31)
5′-UCCGGCCTCGGAAGCUCUCU-3′;
(32)
5′-UCCGGCCTCGGAGTCUCCAU-3′;
(33)
5′-GCGGUATCCATAGTCUCCAU-3′;
(34)
5′-GAUUAAAACAGATTAAUACA-3′;
(35)
5′-UGACAAAACAATAATAACAG-3′;
(36)
5′-CCAACACTTCGTGGGGUCCU-3′;
(37)
5′-GUGUCCTTCATGCTTUGGAU-3′;
(38)
5′-GUCCCAGGCCTCCAGUGUCU-3′;
(39)
5′-UUGGCCTGTGGATGCUUUGU-3′;
(40)
5′-GUCCGTACCTCCACCCACCG-3′;
(41)
5′-GUGUUTTTAATTTTGUAGAG-3′;
(42)
5′-GUCAAACCTAGAAAGAAGCA-3′;
(43)
5′-GGUCUCCTCCACACCCUUCU-3′;
(44)
5′-GUCUCCTCCACACCCUUCUC-3′;
(45)
5′-UGAUGATGCTTGCAGGAGGC-3′;
(46)
5′-UUAAATAATCTAGTTUGAAG-3′;
(47)
5′-AAAGCAGTCTCCATGUCCCA-3′;
(48)
5′-GCGUAGTTTCTCTTCCUCCC-3′;
(49)
5′-UCUGGTCCCATCCCTUCUGC-3′;
(50)
5′-UAUUUCCACATGCCCAGUGU-3′;
(51)
5′-UGUUUCCCCGGAGAGCAAUG-3′;
(52)
5′-UUAGCTCCTTGCCTCGUUCC-3′;
(53)
5′-AUUUCCACATGCCCAGUGUU-3′;
(54)
5′-UGGCGTAGTTTCTCTUCCUC-3′;
(55)
5′-UGACATTTCGTGGCTCCUAC-3′;
(56)
5′-GAAAAGATTATCTTCUUUUA-3′;
(57)
5′-UGUGAAAAGATTATCUUCUU-3′;
(58)
5′-UUGUGAAAAGATTATCUUCU-3′;
(59)
5′-UUUGAAATTCAGAAGAUUUG-3′;
(60)
5′-AAGCAGTCTCCATGTCCCAG-3′;
(61)
5′-AGGAUTAAAACAGATUAAUA-3′;
(62)
5′-AGAUUATCTTCTTTTAAUUU-3′;
(63)
5′-UCCCAACTCTTCTAACUCGU-3′;
(64)
5′-UAAAATAAGGGGAATAGGGG-3′;
(65)
5′-AGUGGCACATACCACACCCU-3′;
(66)
5′-AAGAUTATCTTCTTTUAAUU-3′;
(67)
5′-AGAAAGAAGCAAAGAUUCAA-3′;
(68)
5′-UCCCATCCCTTCTGCUGCCA-3′;
(69)
5′-ACCCUTCTCTCTGGTCCCAU-3′;
(70)
5′-AAUAUCTGCTGCCCACCUUC-3′;
(71)
5′-UCUCUCTGGTCCCATCCCUU-3′;
(72)
5′-AGGCCTCCAGTGTCTUCUCC-3′;
(73)
5′-CAAGCCCCAGCGTTCCUCCG-3′;
(74)
5′-AAAUGTCCTGGCCCTCACUG-3′;
(75)
5′-AAUUUAAAGCATGAAUAUUA-3′;
(76)
5′-AAAAGATTATCTTCTUUUAA-3′;
(77)
5′-AUAUCTGCTGCCCACCUUCU-3′;
(78)
5′-CAGUCTCCATGTCCCAGGCC-3′;
(79)
5′-AAAGATTATCTTCTTUUAAU-3′;
(80)
5′-CCCUUCTCTCTGGTCCCAUC-3′;
(81)
5′-AUGGCCTTTCCGTGCCAAGG-3′;
(82)
5′-GCAUUCCGTGCGGAAGCCUU-3′;
(83)
5′-GGCCGAACTTTCCCGCCUUA-3′;
(84)
5′-GGUCUTGGCTTCGTGGAGCA-3′;
(85)
5′-GGAGCTTCGAGGCCCCAGGC-3′;
(86)
5′-GGUGGTCCACAACCCCUUUC-3′;
(87)
5′-UUCUGGGGACTTCCAGUUUA-3′;
(88)
5′-UGAUUCCAAAGCCAGGGUUA-3′;
(89)
5′-GGGUATCGAAAGAGTCUGGA-3′;
(90)
5′-CUUGCACGTGGCTTCGUCUC-3′;
(91)
5′-GUGUCCTTGCACGTGGCUUC-3′;
(92)
5′-GUAAAAAGCTTTTGAAGUGA-3′;
(93)
5′-AUGCCATCCACTTGAUAGGC-3′;
(94)
5′-UGAAGTAAAAATCAAUAGCG-3′;
(95)
5′-AAGGCCCTTCGCACTUCUUA-3′;
(96)
5′-GUACUCGTCGGCATCCACCA-3′;
(97)
5′-GUCCUTGCACGTGGCUUCGU-3′;
(98)
5′-GCCCATCCATGAGGTCCUGG-3′;
(99)
5′-GUAAAAGGAGAAAACUAUCU-3′;
(100)
5′-UUGAAGTGAAGTAAAAGGAG-3′;
(101)
5′-GUUACTCGTGCCTTGGCAAA-3′;
(102)
5′-GUCCAAGATCAGCAGUCUCA-3′;
(103)
5′-UUCAATGGGAGAATAAAGCA-3′;
(104)
5′-GCAAGGCCCTTCGCACUUCU-3′;
(105)
5′-GGGUCCACCACTAGCCAGUA-3′;
(106)
5′-GUAGAGAAATTATTTUAGGA-3′;
(107)
5′-AUCCACCACGTCGTCCAUGU-3′;
(108)
5′-GGCAUCCACCACGTCGUCCA-3′;
(109)
5′-UUACUTTAAAAGCAAAAGGA-3′;
(110)
5′-UUUGAAGTGAAGTAAAAGGA-3′;
(111)
5′-GAAGUGAAGTAAAAGGAGAA-3′;
(112)
5′-GGCCATCTCTGCTTCUUGGU-3′;
(113)
5′-UGGGCTGGAATCCGAGUUAU-3′;
(114)
5′-GGAGATTTCAGAGCAGCUUC-3′;
(115)
5′-UUUACGGTTTTCAGAAUAUC-3′;
(116)
5′-GCGUGTCTGGAAGCTUCCUU-3′;
(117)
5′-GCUUATTTTAAGCATAUUAA-3′;
(118)
5′-UUAUUTTAAGCATATUAAAA-3′;
(119)
5′-UUCUGCAGCTTCCTTGUCCU-3′;
(120)
5′-AUUACTTTAAAAGCAAAAGG-3′;
(121)
5′-AUUUUAAGCATATTAAAAAG-3′;
(122)
5′-UGUGGCTTGTCCTCAGACAU-3′;
(123)
5′-AAAAGGAGAAAACTAUCUUC-3′;
(124)
5′-GGGUCCATACCCAAGGCAUC-3′;
(125)
5′-ACAGUGTTGAGATACUCGGG-3′;
(126)
5′-GGAUCTGCATGCCCTCAUCU-3′;
(127)
5′-AGUAAAAAGCTTTTGAAGUG-3′;
(128)
5′-GUCGUGGCAAATAGTCCUAG-3′;
(129)
5′-GGAGATCAGATGAGAGGAGC-3′;
(130)
5′-GUGGUTAAGTACATGAGCUC-3′;
(131)
5′-GGACACTTAGCTGTTCCUCG-3′;
(132)
5′-GUCUCTACTGTTACCUCUGA-3′;
(133)
5′-GAGUUCTTCGTAGGCUUCUG-3′;
(134)
5′-AAAGUCAAAAAGAAAAACUG-3′;
(135)
5′-AAAAGTGGGAAATAAAGGUU-3′;
(136)
5′-AGUUUATAGATTTCAAGUAG-3′;
(137)
5′-AAAAAGTGGGAAATAAAGGU-3′;
(138)
5′-UUUAUATTACAAAGCUACUU-3′;
(139)
5′-UGCUATTCATATTTTUAUUU-3′;
(140)
5′-GGUAU-3′;
(141)
5′-[G/A/C][G/A][G/A/C][T/A/C]TC-3′;
(142)
5′-[G/A/C]G[G/A/C][T/A/C]TC-3′;
(143)
5′-GGCTTC-3′;
(144)
5′-GGCATC-3′;
(145
5′-AGCTTC-3′;
(146)
5′-GGAATC-3′;
(147)
5′-CACATC-3′;
(148)
5′-GGCCTC-3′;
(149)
5′-CACTTC-3′;
(150)
5′-AAGATC-3′;
(151)
5′-TGTCCTTGCACGTGGCTTCG-3′;
(152)
5′-TTTGCACACTTCGTACCCAA-3′;
(153)
5′-GTCCACATCCTGTGGCTCGT-3′;
(154)
5′-TGTGATGGCCTCCCATCTCC-3′;
(155)
5′-GGTTTTGGCTGGGATCAAGT-3′;
(156)
5′-GGTGTCCTTGCACGTGGCTT-3′;
(157)
5′-GGTCCATACCCAAGGCATCC-3′;
(158)
5′-GTGTCTTCATCGGCCCTGCC-3′;
(159)
5′-GTCTTGGCTTCGTGGAGCAG-3′;
(160)
5′-GCTGACAAAGATTCACTGGT-3′;
(161)
5′-GAAAGGTTATGCAAGG-3′;
(162)
5′-GACTATACGCGCAATA-3′;
(163)
5′-TGTGATGGCCTCCCAT-3′;
(164)
5′-TCCAACACTTCGTGGG-3′;
(165)
5′-GTGTCTGGAAGCTTCC-3′;
(166)
5′-CTTGAAGCATCGTATC-3′;
(167)
5′-TCGTAGTTGCTTCCTA-3′;
(168)
5′-CGCTTTTCTGTCTGGT-3′;
(169)
5′-GGCTGGAATCCGAGTT-3′;
(170)
5′-GATAGCACCTTCAGCA-3′;
(171)
5′-AGGACTCCAGATGTTT-3′;
(172)
5′-GTGATCTTGACATGCT-3′;
(173)
5′-AGATTTCAGAGCAGCT-3′;
(174)
5′-GGTTACGGCTCAGTAT-3′;
(175)
5′-GTTCAGTCCTGTCCAT-3′;
(176)
5′-AGGTCTTGGCTTCGTG-3′;
(177)
5′-CTGCAGCTTCCTTGTC-3′;
(178)
5′-GTCCTTGCACGTGGCT-3′;
(179)
5′-GTCTCTGGAGCTTCCT-3′;
(180)
5′-GGTCTTGGCTTCGTGG-3′;
(181)
5′-GGUA-3′;
(182)
5′-GUAU-3′;
(183)
5′-GGU-3′;
(184)
5′-GUA-3′;
(185)
5′-GUC-3′;
(186)
5′-GUG-3′;
(187)
5′-GUU-3′;
(188)
5′-GUA-3′;
(189)
5′-GGC-3′;
(190)
5′-AUC-3′;
(191)
5′-GAA-3′;
(192)
5′-GAG-3′;
(193)
5′-GGA-3′;
(194)
5′-GAC-3′;
(195)
5′-GAU-3′;
(196)
5′-AUG-3′;
(197)
5′-GCG-3′;
(198)
5′-UUC-3′;
(199)
5′-GCC-3′;
(200)
5′-GGG-3′;
(201)
5′-AUU-3′;
(202)
5′-GCA-3′;
(203)
5′-AGC-3′;
(204)
5′-AAC-3′;
(205)
5′-CCA-3′;
(206)
5′-UGC-3′;
(207)
5′-CAA-3′;
(208)
5′-CGG-3′;
(209)
5′-ACC-3′;
(210)
5′-AGA-3′;
(211)
5′-TTT-3′;
(212)
5′-TCT-3′
and
(213) a variant of the motifs or sequences of (1) to (212) having at least about 75% sequence identity thereto;
wherein the U may be a T and/or the T may be a U and wherein the oligonucleotide inhibits TLR7 activity when administered to a subject.

83. An oligonucleotide comprising a motif or sequence selected from the group consisting of:
5′-CUUCG-3′;
5′-CUUCGTG-3′;
5′-CUUCGTGGG-3′;
5′-UCG-3′;
5′-UCA-3′;
5′-CGG-3′;
5′-UGG-3′;
5′-CGC-3′;
5′-AGG-3′;
5′-GGA-3′;
5′-GGC-3′;
5′-AGA-3′;
5′-CGA-3′;
5′-UAG-3′;
5′-UCU-3′;
5′-AGC-3′;
5′-GGU-3′;
5′-UGA-3′;
5′-AGU-3′;
5′-ACG-3′;
5′-CGU-3′;
5′-UCC-3′;
5′-GCG-3′;
5′-GGG-3′;
5′-UGU-3′;
5′-UCA-3′;
5′-CUG-3′;
5′-UUG-3′;
5′-UUA-3′;
5′-UGC-3′;
and
a variant of the motifs or sequences thereof having at least about 75% sequence identity thereto;
wherein the U may be a T and/or the T may be a U and wherein the oligonucleotide potentiates TLR8 activity when administered to a subject.

84. An oligonucleotide comprising a motif or sequence selected from the group consisting of:
5′-GAG-3′;
5′-GAC-3′;
5′-GAU-3′;
5′-GAA-3′;
5′-GUC-3′;
5′-GUU-3′;
5′-GUA-3′;
5′-GUG-3′;
5′-AUA-3′;
5′-AUG-3′;
5′-CUU-3′;
5′-AAG-3′;
5′-AUC-3′;
5′-CCC-3′;
5-GCU-3′;
5′-CCU-3′;
5′-CUA-3′;
5′-CUC-3′;
5′-AAC-3′;
and
wherein the U may be a T and/or the T may be a U and wherein the oligonucleotide inhibits TLR8 activity when administered to a subject.

85. An oligonucleotide comprising a motif or sequence selected from the group consisting of:
5′-TAC-3′;
5′-CGC-3′;
5′-GCA-3′;
5′-UGA-3′;
5′-CAG-3′;
5′-UGG-3′;
5′-UCA-3′;
5′-TGA-3′;
5′-CGT-3′;
5′-GAC-3′;
5′-CCA-3′;
5′-TAG-3′;
5′-TGG-3′;
5′-TCA-3′;
5′-TGC-3′;
5′-CAC-3′;
5′-CGG-3′;
5′-CCC-3′;
5′-ACT-3′;
5′-GTA-3′;
5′-GGA-3′;
5′-AAG-3′;
5′-ATA-3′;
5′-GUC-3′;
5′-UCC-3′;
5′-AUC-3′;
5′-CCG-3′;
5′-CAA-3′;
5′-GAU-3′;
5′-CGA-3′;
and
wherein the U may be a T and/or the T may be a U and wherein the oligonucleotide inhibits TLR3 activity when administered to a subject.

86. A composition comprising an oligonucleotide according to any one of claims 76 to 85.

87. The composition of claim 86, which further comprises a pharmaceutically acceptable carrier.

88. A composition consisting essentially of an oligonucleotide according to any one of claims 76 to 85 and a pharmaceutically acceptable carrier.

89. The composition of any one of claims 86 to 88, wherein at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% of the oligonucleotide content of the composition is an oligonucleotide according to any one of claims 76 to 85.

90. The composition of any one of claims 86 to 88, wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% of the oligonucleotide content of the composition is an oligonucleotide according to any one of claims 76 to 85.

91. The composition of any one of claims 86 to 91, wherein at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or at least about 100% of the active pharmaceutical ingrediment in the composition is an oligonucleotide according to any one of claims 76 to 85.

92. A method of reducing expression of a target gene in a cell, the method comprising contacting the cell with an oligonucleotide according to any one of claims 76 to 85 or composition according to any one of claims 86 to 92.

93. A method of treating or preventing a disease, disorder or condition in a subject, the method comprising administering to the subject a therapeutically effective amount of an oligonucleotide according to any one of claims 76 to 85 or composition according to any one of claims 86 to 92 to thereby treat or prevent the disease, disorder or condition in the subject.

94. Use of an oligonucleotide according to any one of claims 76 to 85 or composition according to any one of claims 86 to 92 in the manufacture of a medicament for treating or preventing a disease, disorder or condition in a subject.

95. An oligonucleotide according to any one of claims 76 to 85 or composition according to any one of claims 86 to 92 for use in treating or preventing a disease, disorder or condition in a subject.

96. The method of claim 93, the use of claim 94 or the oligonucleotide of claim 95, wherein the oligonucleotide reduces the expression of a target gene involved in the disease, disorder or condition.

97. The method, use or oligonucleotide of any one of claims 93 to 96, wherein the disease, disorder or condition demonstrates increased, excessive or abnormal cGAS expression, activity and/or signalling.

98. The method, use or oligonucleotide of claim 97, wherein the disease, disorder or condition is selected from the group consisting of Huntington's disease, Parkinson's diseases, motor-neurone disease (MND), amyotrophic lateral sclerosis (ALS), prion disease, frontotemporal dementia, Traumatic brain injury, Alzheimer's disease, Acute pancreatitis, Silica- induced fibrosis, Age dependent macular degeneration, Aicardi-Goutibéres syndrome, myocardial infarction, heart failure, Polyarthritis/foetal and neonatal anaemia, Systemic lupus erythematosus, Acute Kidney Injury, Alcohol-related liver disease, Non-Alcohol-fatty liver disease, silica driven lung inflammation, chronic obstructive pulmonary disease, brain injury after ischemic stroke, sepsis, Non-alcoholic steatohepatitis (NASH), cancer, sickle cell disease, Inflammatory bowel disease, type 2 diabetes mellitus, over-nutrition-induced obesity, COVID-19, hematopoietic disorders, aging-associated inflammation, Cutibacterium acnes Infection, Hepatitis B, posterior-segment eye diseases, arthritis, rheumatoid arthritis, emphysema, colorectal cancer, skin cancer, metastases, and breast cancer.

99. The method, use or oligonucleotide of any one of claims 93 to 96, wherein the disease, disorder or condition demonstrates increased, excessive or abnormal TLR9 expression, activity and/or signalling.

100. The method, use or oligonucleotide of claim 99, wherein the disease, disorder or condition is selected from the group consisting of psoriasis, rheumatoid arthritis, alopecia universalis, acute disseminated encephalomyelitis, Addison's disease, allergy, ankylosing spondylitis, antiphospholipid antibody syndrome, arteriosclerosis, atherosclerosis, autoimmune haemolytic anaemia, autoimmune hepatitis, Bullous pemphigoid, Chagas' disease, chronic obstructive pulmonary disease, coeliac disease, cutaneous lupus erythematosus (CLE), dermatomyositis, diabetes, dilated cardiomyopathy (DC), endometriosis, Goodpasture's syndrome, Graves' disease, Guillain-Barre syndrome, Hashimoto's disease, hidradenitis suppurativa, idiopathic thrombocytopenic purpura, inflammatory bowel disease, interstitial cystitis, morphea, multiple sclerosis (MS), myasthenia gravis, myocarditis, narcolepsy, neuromyotonia, pemphigus, pernicious anaemia, polymyositis, primary biliary cirrhosis, rheumatoid arthritis (RA), schizophrenia, Sjogren's syndrome, systemic lupus erythematosus (SLE), systemic sclerosis, temporal arteritis, vasculitis, vitiligo, vulvodynia, Wegener's granulomatosis, traumatic pain, neuropathic pain and acetaminophen toxicity, breast cancer, cervical squamous cell carcinoma, gastric carcinoma, glioma, hepatocellular carcinoma, lung cancer, melanoma, prostate cancer, recurrent glioblastoma, recurrent non-Hodgkin lymphoma and colorectal cancer.

101. The method, use or oligonucleotide of any one of claims 93 to 96, wherein the disease, disorder or condition demonstrates increased, excessive or abnormal TLR7 expression, activity and/or signalling.

102. The method, use or oligonucleotide of any one of claims 93 to 96, wherein the disease, disorder or condition of the three aforementioned aspects demonstrates increased, excessive or abnormal TLR8 expression, activity and/or signalling.

103. The method, use or oligonucleotide of any one of claims 93 to 96, wherein the disease, disorder or condition of the three aforementioned aspects demonstrates increased, excessive or abnormal TLR3 expression, activity and/or signalling.

104. The method, use or oligonucleotide of any one of claims 93 to 97, wherein the disease, disorder or condition is a senescence-associated disease, disorder or condition, such as aging and/or an aging-related disease, disorder or condition.

105. The method, use or oligonucleotide of any one of claims 93 to 97, 99, or 101 to 103, wherein the disease, disorder or condition is an inflammatory disease, disorder or condition associated with administration of a therapeutic oligonucleotide to the subject.

106. The method, use or oligonucleotide of claim 105, wherein the inflammatory disease, disorder or condition comprises hepatic inflammation.

107. A method of inhibiting cGAS in a subject, including the step of administering to the subject an effective amount of an oligonucleotide according to any one of claims 76 to 78, or a composition according to any one of claims 86 to 91.

108. A method of inhibiting cGAS in a cell, including the step of contacting the cell with an effective amount of an oligonucleotide of any one of claims 76 to 78 or a composition of any one of claims 86 to 91.

109. The method of claim 108, wherein the oligonucleotide inhibits or prevents senescence in the cell.

110. The method of claim 108 or 109, wherein the cell is an immune cell.

111. A method of inhibiting TLR9 in a subject, including the step of administering to the subject an effective amount of an oligonucleotide according to any one of claims 76 or 79 to 81, or a composition according to any one of claims 86 to 91.

112. A method of inhibiting TLR9 in a cell, including the step of contacting the cell with an effective amount of an oligonucleotide according to any one of claims 76 or 79 to 81, or a composition according to any one of claims 86 to 91.

113. A method of inhibiting TLR7 in a subject, including the step of administering to the subject an effective amount of an oligonucleotide according to any one of claims 76 or 82, or a composition according to any one of claims 86 to 91.

114. A method of inhibiting TLR7 in a cell, including the step of contacting the cell with an effective amount of an oligonucleotide according to any one of claims 76 or 82, or a composition according to any one of claims 86 to 91.

115. The method of claim 113 or claim 114, wherein the oligonucleotide at least partly inhibits TLR7 activation in the cell or the subject in response to an RNA molecule.

116. A method of inhibiting TLR7 activation by an RNA molecule in a cell, said method including the step of contacting the cell with an effective amount of an oligonucleotide according to any one of claims 76 or 82, or a composition according to any one of claims 86 to 91.

117. A method of inhibiting TLR7 activation by an RNA molecule in a subject, said method including the step of administering to the subject an effective amount of an oligonucleotide according to any one of claims 76 or 82, or a composition according to any one of claims 86 to 91.

118. A method of inhibiting TLR8 in a subject, including the step of administering to the subject an effective amount of an oligonucleotide according to claim 76 or 84, or a composition according to any one of claims 86 to 91.

119. A method of inhibiting TLR8 in a cell, including the step of contacting the cell with an effective amount of an oligonucleotide according to claim 76 or 84, or a composition according to any one of claims 86 to 91.

120. A method of preventing or inhibiting TLR8 activation by an RNA molecule in a cell, said method including the step of contacting the cell with an effective amount of an oligonucleotide according to claim 76 or 84, or a composition according to any one of claims 86 to 91.

121. A method of preventing inhibiting TLR8 activation by an RNA molecule in a subject, said method including the step of administering to the subject an effective amount of an oligonucleotide according to claim 76 or 84, or a composition according to any one of claims 86 to 91.

122. A method of inhibiting TLR3 in a subject, including the step of administering to the subject an effective amount of an oligonucleotide according to claim 76 or 85, or a composition according to any one of claims 86 to 91.

123. A method of inhibiting TLR3 in a cell, including the step of contacting the cell with an effective amount of an oligonucleotide according to claim 76 or 85, or a composition according to any one of claims 86 to 91.

124. A method of preventing or inhibiting TLR3 activation by an RNA molecule in a cell, said method including the step of contacting the cell with an effective amount of an oligonucleotide according to claim 76 or 85, or a composition according to any one of claims 86 to 91.

125. A method of preventing inhibiting TLR3 activation by an RNA molecule in a subject, said method including the step of administering to the subject an effective amount of an oligonucleotide according to claim 76 or 85, or a composition according to any one of claims 86 to 91.

126. A method of increasing the activity of, or potentiating, TLR8 in a subject, including the step of administering to the subject an effective amount of an oligonucleotide according to claim 76 or 83, or a composition according to any one of claims 86 to 91.

127. A method of increasing the activity of, or potentiating, TLR8 in a cell, including the step of contacting the cell with an effective amount of an oligonucleotide according to claim 76 or 83, or a composition according to any one of claims 86 to 91.

128. A method of increasing or potentiating TLR8 activation by an RNA molecule in a cell, said method including the step of contacting the cell with an effective amount of oligonucleotide according to claim 76 or 83, or a composition according to any one of claims 86 to 91.

129. A method of increasing or potentiating TLR8 activation by an RNA molecule in a subject, said method including the step of administering to the subject an effective amount of an oligonucleotide according to claim 76 or 83, or a composition according to any one of claims 86 to 91.

130. The method of any one of claims 116, 117, 120, 121, 124, 125, 128 or 129, wherein the RNA molecule is an mRNA molecule.

131. The method of claim 130, wherein the mRNA molecule is a component of or included within an immunogenic composition.

132. An immunogenic composition comprising an RNA molecule and an oligonucleotide according to any one of claims 76 to 85.

133. The method of claim 131 or the immunogenic composition of claim 132, wherein the immunogenic composition is an mRNA vaccine composition.

134. The method of any one of claims 107 to 110, 113 to 131 or 133 or the immunogenic composition of claim 132 or claim 133, wherein the oligonucleotide comprises, consists essentially of or consists of a sequence of 5′-GGUAUA-3′, 5′-GGUAU-3′, 5′-GGUA-3′, 5′-GUAU-3′, 5′-GGU-3′ or 5′-GUA-3′, wherein the U may be a T.

135. The steps, features, integers, compositions and/or compounds disclosed herein or indicated in the specification of this application individually or collectively, and any and all combinations of two or more of said steps or features.


SEQUENCE LISTING (599 sequences)

SEQ ID NO: 1 - RNA, 6 nt, Artificial Sequence
  Synthetic construct
rgumyc

SEQ ID NO: 2 - RNA, 6 nt, Artificial Sequence
  Synthetic construct
arkcyc

SEQ ID NO: 3 - RNA, 9 nt, Artificial Sequence
  Synthetic construct
arkcycymu

SEQ ID NO: 4 - RNA, 6 nt, Artificial Sequence
  Synthetic construct
gguaua

SEQ ID NO: 5 - RNA, 6 nt, Artificial Sequence
  Synthetic construct
uguuuc

SEQ ID NO: 6 - RNA, 6 nt, Artificial Sequence
  Synthetic construct
uguguc

SEQ ID NO: 7 - RNA, 6 nt, Artificial Sequence
  Synthetic construct
cguuuc

SEQ ID NO: 8 - RNA, 6 nt, Artificial Sequence
  Synthetic construct
cguguc

SEQ ID NO: 9 - DNA, 20 bp, Artificial Sequence
  Synthetic construct
auggcctttccgtgccaagg

SEQ ID NO: 10 - DNA, 20 bp, Artificial Sequence
  Synthetic construct
uccggcctcggaagcucucu

SEQ ID NO: 11 - DNA, 20 bp, Artificial Sequence
  Synthetic construct
gcauuccgtgcggaagccuu

SEQ ID NO: 12 - DNA, 20 bp, Artificial Sequence
  Synthetic construct
ggccgaactttcccgccuua

SEQ ID NO: 13 - DNA, 20 bp, Artificial Sequence
  Synthetic construct
ggucutggcttcgtggagca

SEQ ID NO: 14 - DNA, 20 bp, Artificial Sequence
  Synthetic construct
ggagcttcgaggccccaggc

SEQ ID NO: 15 - DNA, 20 bp, Artificial Sequence
  Synthetic construct
gguggtccacaaccccuuuc

SEQ ID NO: 16 - DNA, 20 bp, Artificial Sequence
  Synthetic construct
cauuaggtgcagaaaucuuc

SEQ ID NO: 17 - DNA, 20 bp, Artificial Sequence
  Synthetic construct
uucuggggacttccaguuua

SEQ ID NO: 18 - RNA, 20 nt, Artificial Sequence
  Synthetic construct
ugauuccaaagccaggguua

SEQ ID NO: 19 - DNA, 20 bp, Artificial Sequence
  Synthetic construct
cuuuagtcgtagttgcuucc

SEQ ID NO: 20 - DNA, 20 bp, Artificial Sequence
  Synthetic construct
uuaaataatctagttugaag

SEQ ID NO: 21 - DNA, 20 bp, Artificial Sequence
  Synthetic construct
guguccttcatgcttuggau

SEQ ID NO: 22 - RNA, 20 nt, Artificial Sequence
  Synthetic construct
agaaagaagcaaagauucaa

SEQ ID NO: 23 - DNA, 20 bp, Artificial Sequence
  Synthetic construct
agauuatcttcttttaauuu

SEQ ID NO: 24 - DNA, 20 bp, Artificial Sequence
  Synthetic construct
aaaagattatcttctuuuaa

SEQ ID NO: 25 - DNA, 20 bp, Artificial Sequence
  Synthetic construct
gaaaagattatcttcuuuua

SEQ ID NO: 26 - DNA, 20 bp, Artificial Sequence
  Synthetic construct
ugugaaaagattatcuucuu

SEQ ID NO: 27 - DNA, 20 bp, Artificial Sequence
  Synthetic construct
cuugugaaaagattaucuuc

SEQ ID NO: 28 - RNA, 20 nt, Artificial Sequence
  Synthetic construct
gguggccacaggcaacguca

SEQ ID NO: 29 - DNA, 20 bp, Artificial Sequence
  Synthetic construct
ccaugtcccaggcctccagu

SEQ ID NO: 30 - DNA, 20 bp, Artificial Sequence
  Synthetic construct
gcaguctccatgtcccaggc

SEQ ID NO: 31 - DNA, 20 bp, Artificial Sequence
  Synthetic construct
agcagtctccatgtcccagg

SEQ ID NO: 32 - DNA, 20 bp, Artificial Sequence
  Synthetic construct
aguggcacataccacacccu

SEQ ID NO: 33 - DNA, 20 bp, Artificial Sequence
  Synthetic construct
auuuccacatgcccaguguu

SEQ ID NO: 34 - DNA, 20 bp, Artificial Sequence
  Synthetic construct
ggucccatcccttctgcugc

SEQ ID NO: 35 - DNA, 20 bp, Artificial Sequence
  Synthetic construct
ucuggtcccatccctucugc

SEQ ID NO: 36 - DNA, 20 bp, Artificial Sequence
  Synthetic construct
ggguctcctccacacccuuc

SEQ ID NO: 37 - RNA, 20 nt, Artificial Sequence
  Synthetic construct
gcaaggcagagaaacuccag

SEQ ID NO: 38 - DNA, 20 bp, Artificial Sequence
  Synthetic construct
gauggttccagtcccucuuc

SEQ ID NO: 39 - RNA, 20 nt, Artificial Sequence
  Synthetic construct
uguuuccccggagagcaaug

SEQ ID NO: 40 - RNA, 20 nt, Artificial Sequence
  Synthetic construct
agccgaacagaaggagcguc

SEQ ID NO: 41 - DNA, 20 bp, Artificial Sequence
  Synthetic construct
gcguagtttctcttccuccc

SEQ ID NO: 42 - DNA, 20 bp, Artificial Sequence
  Synthetic construct
gcgguatccatgtcccaggc

SEQ ID NO: 43 - DNA, 20 bp, Artificial Sequence
  Synthetic construct
gcgguatacaggtcccaggc

SEQ ID NO: 44 - DNA, 20 bp, Artificial Sequence
  Synthetic construct
gcuguttccatgtcccaggc

SEQ ID NO: 45 - DNA, 20 bp, Artificial Sequence
  Synthetic construct
gcugugtccatgtcccaggc

SEQ ID NO: 46 - DNA, 20 bp, Artificial Sequence
  Synthetic construct
gccguttccatgtcccaggc

SEQ ID NO: 47 - DNA, 20 bp, Artificial Sequence
  Synthetic construct
gccgugtccatgtcccaggc

SEQ ID NO: 48 - DNA, 20 bp, Artificial Sequence
  Synthetic construct
gcgguatccatagtcuccau

SEQ ID NO: 49 - DNA, 20 bp, Artificial Sequence
  Synthetic construct
gcgguatccatcagauaucg

SEQ ID NO: 50 - DNA, 20 bp, Artificial Sequence
  Synthetic construct
cuuuagtcgtagttgucucu

SEQ ID NO: 51 - DNA, 20 bp, Artificial Sequence
  Synthetic construct
uccgggtcgtagttgcuucc

SEQ ID NO: 52 - DNA, 20 bp, Artificial Sequence
  Synthetic construct
uccggcctcggagtcuccau

SEQ ID NO: 53 - DNA, 20 bp, Artificial Sequence
  Synthetic construct
uccggcctcgggagaucucu

SEQ ID NO: 54 - DNA, 20 bp, Artificial Sequence
  Synthetic construct
gguatccccccccccccccc

SEQ ID NO: 55 - DNA, 20 bp, Artificial Sequence
  Synthetic construct
ggauuaaaacagattaauac

SEQ ID NO: 56 - RNA, 5 nt, Artificial Sequence
  Synthetic construct
gguau

SEQ ID NO: 57 - RNA, 4 nt, Artificial Sequence
  Synthetic construct
ggua

SEQ ID NO: 58 - RNA, 4 nt, Artificial Sequence
  Synthetic construct
guau

SEQ ID NO: 59 - RNA, 3 nt, Artificial Sequence
  Synthetic construct
ggu

SEQ ID NO: 60 - RNA, 3 nt, Artificial Sequence
  Synthetic construct
gua

SEQ ID NO: 61 - DNA, 6 bp, Artificial Sequence
  Synthetic construct
tgtctg

SEQ ID NO: 62 - DNA, 4 bp, Artificial Sequence
  Synthetic construct
gtct

SEQ ID NO: 63 - DNA, 6 bp, Artificial Sequence
  Synthetic construct
tctccg

SEQ ID NO: 64 - DNA, 4 bp, Artificial Sequence
  Synthetic construct
ctcc

SEQ ID NO: 65 - DNA, 8 bp, Artificial Sequence
  Synthetic construct
rmagsytm

SEQ ID NO: 66 - DNA, 8 bp, Artificial Sequence
  Synthetic construct
aaaggtta

SEQ ID NO: 67 - DNA, 8 bp, Artificial Sequence
  Synthetic construct
gaagcttc

SEQ ID NO: 68 - DNA, 8 bp, Artificial Sequence
  Synthetic construct
gcaggctc

SEQ ID NO: 69 - DNA, 6 bp, Artificial Sequence
  Synthetic construct
arggtt

SEQ ID NO: 70 - DNA, 6 bp, Artificial Sequence
  Synthetic construct
agggtt

SEQ ID NO: 71 - DNA, 6 bp, Artificial Sequence
  Synthetic construct
aaggtt

SEQ ID NO: 72 - DNA, 4 bp, Artificial Sequence
  Synthetic construct
ggtt

SEQ ID NO: 73 - DNA, 8 bp, Artificial Sequence
  Synthetic construct
rgctyysw

SEQ ID NO: 74 - DNA, 8 bp, Artificial Sequence
  Synthetic construct
agcttcct

SEQ ID NO: 75 - DNA, 8 bp, Artificial Sequence
  Synthetic construct
agcttcga

SEQ ID NO: 76 - DNA, 8 bp, Artificial Sequence
  Synthetic construct
ggcttcgt

SEQ ID NO: 77 - DNA, 8 bp, Artificial Sequence
  Synthetic construct
tgcttcct

SEQ ID NO: 78 - DNA, 8 bp, Artificial Sequence
  Synthetic construct
agctctct

SEQ ID NO: 79 - DNA, 4 bp, Artificial Sequence
  Synthetic construct
gstt

SEQ ID NO: 80 - DNA, 4 bp, Artificial Sequence
  Synthetic construct
gctt

SEQ ID NO: 81 - DNA, 20 bp, Artificial Sequence
  Synthetic construct
cggaggtcttggcttcgtgg

SEQ ID NO: 82 - DNA, 20 bp, Artificial Sequence
  Synthetic construct
aggtcttggcttcgtggagc

SEQ ID NO: 83 - DNA, 20 bp, Artificial Sequence
  Synthetic construct
gggaaaggttatgcaaggtc

SEQ ID NO: 84 - DNA, 20 bp, Artificial Sequence
  Synthetic construct
ctgtgatcttgacatgctgc

SEQ ID NO: 85 - DNA, 20 bp, Artificial Sequence
  Synthetic construct
actgactgtcttgagggttc

SEQ ID NO: 86 - DNA, 20 bp, Artificial Sequence
  Synthetic construct
gcgtgtctggaagcttcctt

SEQ ID NO: 87 - DNA, 20 bp, Artificial Sequence
  Synthetic construct
gagtctctggagcttcctct

SEQ ID NO: 88 - DNA, 20 bp, Artificial Sequence
  Synthetic construct
agtcgtagttgcttcctaac

SEQ ID NO: 89 - DNA, 20 bp, Artificial Sequence
  Synthetic construct
gtcttggcttcgtggagcag

SEQ ID NO: 90 - DNA, 20 bp, Artificial Sequence
  Synthetic construct
ttggctcggcttgcctactt

SEQ ID NO: 91 - DNA, 20 bp, Artificial Sequence
  Synthetic construct
acagtgttgagatactcggg

SEQ ID NO: 92 - DNA, 20 bp, Artificial Sequence
  Synthetic construct
tcgcacttcagtctgagcag

SEQ ID NO: 93 - DNA, 20 bp, Artificial Sequence
  Synthetic construct
ggtgtccttgcacgtggctt

SEQ ID NO: 94 - DNA, 20 bp, Artificial Sequence
  Synthetic construct
tttgcacacttcgtacccaa

SEQ ID NO: 95 - DNA, 20 bp, Artificial Sequence
  Synthetic construct
gctgacaaagattcactggt

SEQ ID NO: 96 - DNA, 20 bp, Artificial Sequence
  Synthetic construct
gcggaggtcttggcttcgtg

SEQ ID NO: 97 - DNA, 16 bp, Artificial Sequence
  Synthetic construct
ccaagatcagcagtct

SEQ ID NO: 98 - DNA, 16 bp, Artificial Sequence
  Synthetic construct
cttgaagcatcgtatc

SEQ ID NO: 99 - DNA, 16 bp, Artificial Sequence
  Synthetic construct
gcacacttcgtaccca

SEQ ID NO: 100 - DNA, 16 bp, Artificial Sequence
  Synthetic construct
gatagcaccttcagca

SEQ ID NO: 101 - DNA, 16 bp, Artificial Sequence
  Synthetic construct
cgtattatagccgatt

SEQ ID NO: 102 - DNA, 16 bp, Artificial Sequence
  Synthetic construct
gcaggctcagtgatgt

SEQ ID NO: 103 - DNA, 16 bp, Artificial Sequence
  Synthetic construct
gaaaggttatgcaagg

SEQ ID NO: 104 - DNA, 16 bp, Artificial Sequence
  Synthetic construct
atggcctcccatctcc

SEQ ID NO: 105 - DNA, 16 bp, Artificial Sequence
  Synthetic construct
cgcttttctgtctggt

SEQ ID NO: 106 - DNA, 16 bp, Artificial Sequence
  Synthetic construct
gtgtctggaagcttcc

SEQ ID NO: 107 - DNA, 16 bp, Artificial Sequence
  Synthetic construct
tggcctcccatctcct

SEQ ID NO: 108 - DNA, 16 bp, Artificial Sequence
  Synthetic construct
atctggcagcccatca

SEQ ID NO: 109 - DNA, 16 bp, Artificial Sequence
  Synthetic construct
gaggtcttggcttcgt

SEQ ID NO: 110 - DNA, 16 bp, Artificial Sequence
  Synthetic construct
acacttcgtggggtcc

SEQ ID NO: 111 - DNA, 16 bp, Artificial Sequence
  Synthetic construct
ttcgtggggtcctttt

SEQ ID NO: 112 - DNA, 16 bp, Artificial Sequence
  Synthetic construct
ccacttggcagaccat

SEQ ID NO: 113 - DNA, 16 bp, Artificial Sequence
  Synthetic construct
ccatccatgaggtcct

SEQ ID NO: 114 - DNA, 16 bp, Artificial Sequence
  Synthetic construct
tccaacacttcgtggg

SEQ ID NO: 115 - DNA, 16 bp, Artificial Sequence
  Synthetic construct
tcttcatcggccctgc

SEQ ID NO: 116 - DNA, 16 bp, Artificial Sequence
  Synthetic construct
ccagcaggtcagcaaa

SEQ ID NO: 117 - DNA, 16 bp, Artificial Sequence
  Synthetic construct
cgcttttctctccggt

SEQ ID NO: 118 - DNA, 23 bp, Artificial Sequence
  Synthetic construct
gguauaggactccagatguuucc

SEQ ID NO: 119 - DNA, 6 bp, Artificial Sequence
  Synthetic construct
gguatc

SEQ ID NO: 120 - RNA, 6 nt, Artificial Sequence
  Synthetic construct
gguauc

SEQ ID NO: 121 - RNA, 6 nt, Artificial Sequence
  Synthetic construct
agucuc

SEQ ID NO: 122 - RNA, 6 nt, Artificial Sequence
  Synthetic construct
gguccc

SEQ ID NO: 123 - RNA, 6 nt, Artificial Sequence
  Synthetic construct
ggucuc

SEQ ID NO: 124 - RNA, 6 nt, Artificial Sequence
  Synthetic construct
aagcuc

SEQ ID NO: 125 - RNA, 6 nt, Artificial Sequence
  Synthetic construct
aguccc

SEQ ID NO: 126 - DNA, 6 bp, Artificial Sequence
  Synthetic construct
aguctc

SEQ ID NO: 127 - DNA, 6 bp, Artificial Sequence
  Synthetic construct
agtctc

SEQ ID NO: 128 - DNA, 6 bp, Artificial Sequence
  Synthetic construct
gguctc

SEQ ID NO: 129 - DNA, 6 bp, Artificial Sequence
  Synthetic construct
agtccc

SEQ ID NO: 130 - DNA, 6 bp, Artificial Sequence
  Synthetic construct
gguata

SEQ ID NO: 131 - DNA, 6 bp, Artificial Sequence
  Synthetic construct
uguttc

SEQ ID NO: 132 - DNA, 6 bp, Artificial Sequence
  Synthetic construct
ugugtc

SEQ ID NO: 133 - DNA, 6 bp, Artificial Sequence
  Synthetic construct
cguttc

SEQ ID NO: 134 - DNA, 6 bp, Artificial Sequence
  Synthetic construct
cgugtc

SEQ ID NO: 135 - DNA, 5 bp, Artificial Sequence
  Synthetic construct
gguat

SEQ ID NO: 136 - DNA, 4 bp, Artificial Sequence
  Synthetic construct
guat

SEQ ID NO: 137 - DNA, 20 bp, Artificial Sequence
  Synthetic construct
gcgguauccatgtcccaggc

SEQ ID NO: 138 - DNA, 20 bp, Artificial Sequence
  Synthetic construct
tccggcctcggaagctctct

SEQ ID NO: 139 - DNA, 20 bp, Artificial Sequence
  Synthetic construct
ggtcttggcttcgtggagca

SEQ ID NO: 140 - RNA, 6 nt, Artificial Sequence
  Synthetic construct
ycuucu

SEQ ID NO: 141 - DNA, 20 bp, Artificial Sequence
  Synthetic construct
cacccttctctctgguccca

SEQ ID NO: 142 - DNA, 20 bp, Artificial Sequence
  Synthetic construct
ccuuctctctggtcccaucc

SEQ ID NO: 143 - DNA, 20 bp, Artificial Sequence
  Synthetic construct
ucucuggtcccatcccuucu

SEQ ID NO: 144 - DNA, 20 bp, Artificial Sequence
  Synthetic construct
auauctgctgcccaccuucu

SEQ ID NO: 145 - DNA, 20 bp, Artificial Sequence
  Synthetic construct
gucccatcccttctgcugcc

SEQ ID NO: 146 - DNA, 20 bp, Artificial Sequence
  Synthetic construct
gucucctccacacccuucuc

SEQ ID NO: 147 - DNA, 20 bp, Artificial Sequence
  Synthetic construct
caggcctccagtgtcuucuc

SEQ ID NO: 148 - DNA, 20 bp, Artificial Sequence
  Synthetic construct
ucccaactcttctaacucgu

SEQ ID NO: 149 - RNA, 6 nt, Artificial Sequence
  Synthetic construct
ccuucu

SEQ ID NO: 150 - RNA, 6 nt, Artificial Sequence
  Synthetic construct
ucuucu

SEQ ID NO: 151 - RNA, 20 nt, Artificial Sequence
  Synthetic construct
gcgguauccaugucccaggc

SEQ ID NO: 152 - DNA, 6 bp, Artificial Sequence
  Synthetic construct
gsccts

SEQ ID NO: 153 - RNA, 3 nt, Artificial Sequence
  Synthetic construct
cuu

SEQ ID NO: 154 - DNA, 20 bp, Artificial Sequence
  Synthetic construct
cuucuctctggtcccauccc

SEQ ID NO: 155 - DNA, 20 bp, Artificial Sequence
  Synthetic construct
cccuuctctctggtcccauc

SEQ ID NO: 156 - DNA, 20 bp, Artificial Sequence
  Synthetic construct
acccutctctctggtcccau

SEQ ID NO: 157 - DNA, 20 bp, Artificial Sequence
  Synthetic construct
cuuccacaatcaagacauuc

SEQ ID NO: 158 - DNA, 20 bp, Artificial Sequence
  Synthetic construct
cuucgtggggtccttuucac

SEQ ID NO: 159 - DNA, 20 bp, Artificial Sequence
  Synthetic construct
cacuucgtggggtccuuuuc

SEQ ID NO: 160 - DNA, 20 bp, Artificial Sequence
  Synthetic construct
ccaacacttcgtgggguccu

SEQ ID NO: 161 - DNA, 20 bp, Artificial Sequence
  Synthetic construct
uuggcctgtggatgcuuugu

SEQ ID NO: 162 - DNA, 20 bp, Artificial Sequence
  Synthetic construct
aaaugtcctggccctcacug

SEQ ID NO: 163 - DNA, 20 bp, Artificial Sequence
  Synthetic construct
uccggcctcggcagauaucg

SEQ ID NO: 164 - RNA, 20 nt, Artificial Sequence
  Synthetic construct
ccuagaaagaagcaaagauu

SEQ ID NO: 165 - DNA, 20 bp, Artificial Sequence
  Synthetic construct
gauuaaaacagattaauaca

SEQ ID NO: 166 - DNA, 20 bp, Artificial Sequence
  Synthetic construct
aauuuaaagcatgaauauua

SEQ ID NO: 167 - DNA, 20 bp, Artificial Sequence
  Synthetic construct
ugacaaaacaataataacag

SEQ ID NO: 168 - RNA, 3 nt, Artificial Sequence
  Synthetic construct
aca

SEQ ID NO: 169 - RNA, 3 nt, Artificial Sequence
  Synthetic construct
cac

SEQ ID NO: 170 - DNA, 20 bp, Artificial Sequence
  Synthetic construct
acacttcgtggggtcctttt

SEQ ID NO: 171 - DNA, 20 bp, Artificial Sequence
  Synthetic construct
tcaaaggactgaggaaaggg

SEQ ID NO: 172 - DNA, 20 bp, Artificial Sequence
  Synthetic construct
atccaacacttcgtggggtc

SEQ ID NO: 173 - DNA, 20 bp, Artificial Sequence
  Synthetic construct
gcccatccatgaggtcctgg

SEQ ID NO: 174 - DNA, 20 bp, Artificial Sequence
  Synthetic construct
gggtatcgaaagagtctgga

SEQ ID NO: 175 - DNA, 20 bp, Artificial Sequence
  Synthetic construct
ggttttggctgggatcaagt

SEQ ID NO: 176 - DNA, 20 bp, Artificial Sequence
  Synthetic construct
gcgactatacgcgcaatatg

SEQ ID NO: 177 - DNA, 20 bp, Artificial Sequence
  Synthetic construct
aacacttcgtggggtccttt

SEQ ID NO: 178 - DNA, 20 bp, Artificial Sequence
  Synthetic construct
gtccaagatcagcagtctca

SEQ ID NO: 179 - DNA, 20 bp, Artificial Sequence
  Synthetic construct
tgggctggaatccgagttat

SEQ ID NO: 180 - DNA, 20 bp, Artificial Sequence
  Synthetic construct
cggcatccaccacgtcgtcc

SEQ ID NO: 181 - DNA, 20 bp, Artificial Sequence
  Synthetic construct
gcgtattatagccgattaac

SEQ ID NO: 182 - DNA, 20 bp, Artificial Sequence
  Synthetic construct
ggaggtcttggcttcgtgga

SEQ ID NO: 183 - DNA, 20 bp, Artificial Sequence
  Synthetic construct
tgggttacggctcagtatgg

SEQ ID NO: 184 - DNA, 20 bp, Artificial Sequence
  Synthetic construct
ccgccatgtttcttcttgga

SEQ ID NO: 185 - DNA, 16 bp, Artificial Sequence
  Synthetic construct
agcttcgaggccccag

SEQ ID NO: 186 - DNA, 16 bp, Artificial Sequence
  Synthetic construct
gccatgtttcttcttg

SEQ ID NO: 187 - DNA, 16 bp, Artificial Sequence
  Synthetic construct
cacttcgtggggtcct

SEQ ID NO: 188 - DNA, 16 bp, Artificial Sequence
  Synthetic construct
cggcctcggaagctct

SEQ ID NO: 189 - DNA, 16 bp, Artificial Sequence
  Synthetic construct
tgcacacttcgtaccc

SEQ ID NO: 190 - DNA, 16 bp, Artificial Sequence
  Synthetic construct
ccacatcctgtggctc

SEQ ID NO: 191 - DNA, 16 bp, Artificial Sequence
  Synthetic construct
ctgcagcttccttgtc

SEQ ID NO: 192 - DNA, 16 bp, Artificial Sequence
  Synthetic construct
acttcgtggggtcctt

SEQ ID NO: 193 - DNA, 16 bp, Artificial Sequence
  Synthetic construct
cccacttggcagacca

SEQ ID NO: 194 - DNA, 16 bp, Artificial Sequence
  Synthetic construct
gtcccctgttgactgg

SEQ ID NO: 195 - DNA, 16 bp, Artificial Sequence
  Synthetic construct
acgttcagtcctgtcc

SEQ ID NO: 196 - DNA, 16 bp, Artificial Sequence
  Synthetic construct
ggtcattacaatagct

SEQ ID NO: 197 - DNA, 16 bp, Artificial Sequence
  Synthetic construct
tcgtggggtccttttc

SEQ ID NO: 198 - DNA, 16 bp, Artificial Sequence
  Synthetic construct
tgctcctcggtctccc

SEQ ID NO: 199 - DNA, 16 bp, Artificial Sequence
  Synthetic construct
gcatccaccacgtcgt

SEQ ID NO: 200 - DNA, 16 bp, Artificial Sequence
  Synthetic construct
cttcgtggggtccttt

SEQ ID NO: 201 - DNA, 16 bp, Artificial Sequence
  Synthetic construct
aggcccttcgcacttc

SEQ ID NO: 202 - DNA, 3 bp, Artificial Sequence
  Synthetic construct
cut

SEQ ID NO: 203 - DNA, 3 bp, Artificial Sequence
  Synthetic construct
ctt

SEQ ID NO: 204 - DNA, 6 bp, Artificial Sequence
  Synthetic construct
ggcctc

SEQ ID NO: 205 - DNA, 6 bp, Artificial Sequence
  Synthetic construct
ggcctg

SEQ ID NO: 206 - DNA, 6 bp, Artificial Sequence
  Synthetic construct
gccctc

SEQ ID NO: 207 - RNA, 6 nt, Artificial Sequence
  Synthetic construct
ggccuc

SEQ ID NO: 208 - DNA, 20 bp, Artificial Sequence
  Synthetic construct
caacacttcgtggggtcctt

SEQ ID NO: 209 - DNA, 20 bp, Artificial Sequence
  Synthetic construct
ccaacacttcgtggggtcct

SEQ ID NO: 210 - DNA, 20 bp, Artificial Sequence
  Synthetic construct
ggtggtccacaacccctttc

SEQ ID NO: 211 - DNA, 13 bp, Artificial Sequence
  Synthetic construct
kwdaamrvakbaw

SEQ ID NO: 212 - RNA, 9 nt, Artificial Sequence
  Synthetic construct
arkcycymu

SEQ ID NO: 213 - DNA, 20 bp, Artificial Sequence
  Synthetic construct
uucuctctggtcccaucccu

SEQ ID NO: 214 - DNA, 20 bp, Artificial Sequence
  Synthetic construct
guucagtcagatcgcuggga

SEQ ID NO: 215 - DNA, 20 bp, Artificial Sequence
  Synthetic construct
augacatttcgtggcuccua

SEQ ID NO: 216 - DNA, 20 bp, Artificial Sequence
  Synthetic construct
ucuccatgtcccaggccucc

SEQ ID NO: 217 - DNA, 20 bp, Artificial Sequence
  Synthetic construct
agucuccatgtcccaggccu

SEQ ID NO: 218 - DNA, 20 bp, Artificial Sequence
  Synthetic construct
gucccaggcctccagugucu

SEQ ID NO: 219 - DNA, 20 bp, Artificial Sequence
  Synthetic construct
guccgtacctccacccaccg

SEQ ID NO: 220 - DNA, 20 bp, Artificial Sequence
  Synthetic construct
guguutttaattttguagag

SEQ ID NO: 221 - DNA, 20 bp, Artificial Sequence
  Synthetic construct
gucaaacctagaaagaagca

SEQ ID NO: 222 - DNA, 20 bp, Artificial Sequence
  Synthetic construct
ggucucctccacacccuucu

SEQ ID NO: 223 - DNA, 20 bp, Artificial Sequence
  Synthetic construct
ugaugatgcttgcaggaggc

SEQ ID NO: 224 - DNA, 20 bp, Artificial Sequence
  Synthetic construct
aaagcagtctccatguccca

SEQ ID NO: 225 - DNA, 20 bp, Artificial Sequence
  Synthetic construct
uauuuccacatgcccagugu

SEQ ID NO: 226 - DNA, 20 bp, Artificial Sequence
  Synthetic construct
uuagctccttgcctcguucc

SEQ ID NO: 227 - DNA, 20 bp, Artificial Sequence
  Synthetic construct
uggcgtagtttctctuccuc

SEQ ID NO: 228 - DNA, 20 bp, Artificial Sequence
  Synthetic construct
ugacatttcgtggctccuac

SEQ ID NO: 229 - DNA, 20 bp, Artificial Sequence
  Synthetic construct
uugugaaaagattatcuucu

SEQ ID NO: 230 - DNA, 20 bp, Artificial Sequence
  Synthetic construct
uuugaaattcagaagauuug

SEQ ID NO: 231 - DNA, 20 bp, Artificial Sequence
  Synthetic construct
aagcagtctccatgtcccag

SEQ ID NO: 232 - DNA, 20 bp, Artificial Sequence
  Synthetic construct
aggautaaaacagatuaaua

SEQ ID NO: 233 - DNA, 20 bp, Artificial Sequence
  Synthetic construct
uaaaataaggggaatagggg

SEQ ID NO: 234 - DNA, 20 bp, Artificial Sequence
  Synthetic construct
aagautatcttctttuaauu

SEQ ID NO: 235 - DNA, 20 bp, Artificial Sequence
  Synthetic construct
ucccatcccttctgcugcca

SEQ ID NO: 236 - DNA, 20 bp, Artificial Sequence
  Synthetic construct
aauauctgctgcccaccuuc

SEQ ID NO: 237 - DNA, 20 bp, Artificial Sequence
  Synthetic construct
ucucuctggtcccatcccuu

SEQ ID NO: 238 - DNA, 20 bp, Artificial Sequence
  Synthetic construct
aggcctccagtgtctucucc

SEQ ID NO: 239 - DNA, 20 bp, Artificial Sequence
  Synthetic construct
caagccccagcgttccuccg

SEQ ID NO: 240 - DNA, 20 bp, Artificial Sequence
  Synthetic construct
caguctccatgtcccaggcc

SEQ ID NO: 241 - DNA, 20 bp, Artificial Sequence
  Synthetic construct
aaagattatcttcttuuaau

SEQ ID NO: 242 - DNA, 20 bp, Artificial Sequence
  Synthetic construct
ggguatcgaaagagtcugga

SEQ ID NO: 243 - DNA, 20 bp, Artificial Sequence
  Synthetic construct
cuugcacgtggcttcgucuc

SEQ ID NO: 244 - DNA, 20 bp, Artificial Sequence
  Synthetic construct
guguccttgcacgtggcuuc

SEQ ID NO: 245 - DNA, 20 bp, Artificial Sequence
  Synthetic construct
guaaaaagcttttgaaguga

SEQ ID NO: 246 - DNA, 20 bp, Artificial Sequence
  Synthetic construct
augccatccacttgauaggc

SEQ ID NO: 247 - DNA, 20 bp, Artificial Sequence
  Synthetic construct
ugaagtaaaaatcaauagcg

SEQ ID NO: 248 - DNA, 20 bp, Artificial Sequence
  Synthetic construct
aaggcccttcgcactucuua

SEQ ID NO: 249 - DNA, 20 bp, Artificial Sequence
  Synthetic construct
guacucgtcggcatccacca

SEQ ID NO: 250 - DNA, 20 bp, Artificial Sequence
  Synthetic construct
guccutgcacgtggcuucgu

SEQ ID NO: 251 - DNA, 20 bp, Artificial Sequence
  Synthetic construct
gcccatccatgaggtccugg

SEQ ID NO: 252 - RNA, 20 nt, Artificial Sequence
  Synthetic construct
guaaaaggagaaaacuaucu

SEQ ID NO: 253 - DNA, 20 bp, Artificial Sequence
  Synthetic construct
uugaagtgaagtaaaaggag

SEQ ID NO: 254 - DNA, 20 bp, Artificial Sequence
  Synthetic construct
guuactcgtgccttggcaaa

SEQ ID NO: 255 - DNA, 20 bp, Artificial Sequence
  Synthetic construct
guccaagatcagcagucuca

SEQ ID NO: 256 - DNA, 20 bp, Artificial Sequence
  Synthetic construct
uucaatgggagaataaagca

SEQ ID NO: 257 - DNA, 20 bp, Artificial Sequence
  Synthetic construct
gcaaggcccttcgcacuucu

SEQ ID NO: 258 - DNA, 20 bp, Artificial Sequence
  Synthetic construct
ggguccaccactagccagua

SEQ ID NO: 259 - DNA, 20 bp, Artificial Sequence
  Synthetic construct
guagagaaattatttuagga

SEQ ID NO: 260 - DNA, 20 bp, Artificial Sequence
  Synthetic construct
auccaccacgtcgtccaugu

SEQ ID NO: 261 - DNA, 20 bp, Artificial Sequence
  Synthetic construct
ggcauccaccacgtcgucca

SEQ ID NO: 262 - DNA, 20 bp, Artificial Sequence
  Synthetic construct
uuacuttaaaagcaaaagga

SEQ ID NO: 263 - DNA, 20 bp, Artificial Sequence
  Synthetic construct
uuugaagtgaagtaaaagga

SEQ ID NO: 264 - DNA, 20 bp, Artificial Sequence
  Synthetic construct
gaagugaagtaaaaggagaa

SEQ ID NO: 265 - DNA, 20 bp, Artificial Sequence
  Synthetic construct
ggccatctctgcttcuuggu

SEQ ID NO: 266 - DNA, 20 bp, Artificial Sequence
  Synthetic construct
ugggctggaatccgaguuau

SEQ ID NO: 267 - DNA, 20 bp, Artificial Sequence
  Synthetic construct
ggagatttcagagcagcuuc

SEQ ID NO: 268 - DNA, 20 bp, Artificial Sequence
  Synthetic construct
uuuacggttttcagaauauc

SEQ ID NO: 269 - DNA, 20 bp, Artificial Sequence
  Synthetic construct
gcgugtctggaagctuccuu

SEQ ID NO: 270 - DNA, 20 bp, Artificial Sequence
  Synthetic construct
gcuuattttaagcatauuaa

SEQ ID NO: 271 - DNA, 20 bp, Artificial Sequence
  Synthetic construct
uuauuttaagcatatuaaaa

SEQ ID NO: 272 - DNA, 20 bp, Artificial Sequence
  Synthetic construct
uucugcagcttccttguccu

SEQ ID NO: 273 - DNA, 20 bp, Artificial Sequence
  Synthetic construct
auuactttaaaagcaaaagg

SEQ ID NO: 274 - DNA, 20 bp, Artificial Sequence
  Synthetic construct
auuuuaagcatattaaaaag

SEQ ID NO: 275 - DNA, 20 bp, Artificial Sequence
  Synthetic construct
uguggcttgtcctcagacau

SEQ ID NO: 276 - DNA, 20 bp, Artificial Sequence
  Synthetic construct
aaaaggagaaaactaucuuc

SEQ ID NO: 277 - DNA, 20 bp, Artificial Sequence
  Synthetic construct
ggguccatacccaaggcauc

SEQ ID NO: 278 - DNA, 20 bp, Artificial Sequence
  Synthetic construct
acagugttgagatacucggg

SEQ ID NO: 279 - DNA, 20 bp, Artificial Sequence
  Synthetic construct
ggauctgcatgccctcaucu

SEQ ID NO: 280 - DNA, 20 bp, Artificial Sequence
  Synthetic construct
aguaaaaagcttttgaagug

SEQ ID NO: 281 - DNA, 20 bp, Artificial Sequence
  Synthetic construct
gucguggcaaatagtccuag

SEQ ID NO: 282 - DNA, 20 bp, Artificial Sequence
  Synthetic construct
ggagatcagatgagaggagc

SEQ ID NO: 283 - DNA, 20 bp, Artificial Sequence
  Synthetic construct
guggutaagtacatgagcuc

SEQ ID NO: 284 - DNA, 20 bp, Artificial Sequence
  Synthetic construct
ggacacttagctgttccucg

SEQ ID NO: 285 - DNA, 20 bp, Artificial Sequence
  Synthetic construct
gucuctactgttaccucuga

SEQ ID NO: 286 - DNA, 20 bp, Artificial Sequence
  Synthetic construct
gaguucttcgtaggcuucug

SEQ ID NO: 287 - RNA, 20 nt, Artificial Sequence
  Synthetic construct
aaagucaaaaagaaaaacug

SEQ ID NO: 288 - DNA, 20 bp, Artificial Sequence
  Synthetic construct
aaaagtgggaaataaagguu

SEQ ID NO: 289 - DNA, 20 bp, Artificial Sequence
  Synthetic construct
aguuuatagatttcaaguag

SEQ ID NO: 290 - DNA, 20 bp, Artificial Sequence
  Synthetic construct
aaaaagtgggaaataaaggu

SEQ ID NO: 291 - DNA, 20 bp, Artificial Sequence
  Synthetic construct
uuuauattacaaagcuacuu

SEQ ID NO: 292 - DNA, 20 bp, Artificial Sequence
  Synthetic construct
ugcuattcatattttuauuu

SEQ ID NO: 293 - DNA, 6 bp, Artificial Sequence
  Synthetic construct
vrvhtc

SEQ ID NO: 294 - DNA, 6 bp, Artificial Sequence
  Synthetic construct
vgvhtc

SEQ ID NO: 295 - DNA, 6 bp, Artificial Sequence
  Synthetic construct
ggcttc

SEQ ID NO: 296 - DNA, 6 bp, Artificial Sequence
  Synthetic construct
ggcatc

SEQ ID NO: 297 - DNA, 6 bp, Artificial Sequence
  Synthetic construct
agcttc

SEQ ID NO: 298 - DNA, 6 bp, Artificial Sequence
  Synthetic construct
ggaatc

SEQ ID NO: 299 - DNA, 6 bp, Artificial Sequence
  Synthetic construct
cacatc

SEQ ID NO: 300 - DNA, 6 bp, Artificial Sequence
  Synthetic construct
cacttc

SEQ ID NO: 301 - DNA, 6 bp, Artificial Sequence
  Synthetic construct
aagatc

SEQ ID NO: 302 - DNA, 20 bp, Artificial Sequence
  Synthetic construct
tgtccttgcacgtggcttcg

SEQ ID NO: 303 - DNA, 20 bp, Artificial Sequence
  Synthetic construct
gtccacatcctgtggctcgt

SEQ ID NO: 304 - DNA, 20 bp, Artificial Sequence
  Synthetic construct
tgtgatggcctcccatctcc

SEQ ID NO: 305 - DNA, 20 bp, Artificial Sequence
  Synthetic construct
ggtccatacccaaggcatcc

SEQ ID NO: 306 - DNA, 20 bp, Artificial Sequence
  Synthetic construct
gtgtcttcatcggccctgcc

SEQ ID NO: 307 - DNA, 16 bp, Artificial Sequence
  Synthetic construct
gactatacgcgcaata

SEQ ID NO: 308 - DNA, 16 bp, Artificial Sequence
  Synthetic construct
tgtgatggcctcccat

SEQ ID NO: 309 - DNA, 16 bp, Artificial Sequence
  Synthetic construct
tcgtagttgcttccta

SEQ ID NO: 310 - DNA, 16 bp, Artificial Sequence
  Synthetic construct
ggctggaatccgagtt

SEQ ID NO: 311 - DNA, 16 bp, Artificial Sequence
  Synthetic construct
aggactccagatgttt

SEQ ID NO: 312 - DNA, 16 bp, Artificial Sequence
  Synthetic construct
gtgatcttgacatgct

SEQ ID NO: 313 - DNA, 16 bp, Artificial Sequence
  Synthetic construct
agatttcagagcagct

SEQ ID NO: 314 - DNA, 16 bp, Artificial Sequence
  Synthetic construct
ggttacggctcagtat

SEQ ID NO: 315 - DNA, 16 bp, Artificial Sequence
  Synthetic construct
gttcagtcctgtccat

SEQ ID NO: 316 - DNA, 16 bp, Artificial Sequence
  Synthetic construct
aggtcttggcttcgtg

SEQ ID NO: 317 - DNA, 16 bp, Artificial Sequence
  Synthetic construct
gtccttgcacgtggct

SEQ ID NO: 318 - DNA, 16 bp, Artificial Sequence
  Synthetic construct
gtctctggagcttcct

SEQ ID NO: 319 - DNA, 16 bp, Artificial Sequence
  Synthetic construct
ggtcttggcttcgtgg

SEQ ID NO: 320 - DNA, 20 bp, Artificial Sequence
  Synthetic construct
ggcatccaccacgtcgtcca

SEQ ID NO: 321 - DNA, 20 bp, Artificial Sequence
  Synthetic construct
gtccttgcacgtggcttcgt

SEQ ID NO: 322 - DNA, 20 bp, Artificial Sequence
  Synthetic construct
ggagatttcagagcagcttc

SEQ ID NO: 323 - DNA, 20 bp, Artificial Sequence
  Synthetic construct
ttctgcagcttccttgtcct

SEQ ID NO: 324 - DNA, 20 bp, Artificial Sequence
  hIFIT1-FWD
tcaccagatagggctttgct

SEQ ID NO: 325 - DNA, 20 bp, Artificial Sequence
  hIFIT1-REV
cacctcaaatgtgggctttt

SEQ ID NO: 326 - DNA, 20 bp, Artificial Sequence
  h18S-FWD
cggctaccacatccaaggaa

SEQ ID NO: 327 - DNA, 18 bp, Artificial Sequence
  h18S-REV
gctggaattaccgcggct

SEQ ID NO: 328 - DNA, 21 bp, Artificial Sequence
  hIFI44-FWD
atggcagtgacaactcgtttg

SEQ ID NO: 329 - DNA, 23 bp, Artificial Sequence
  HIFI44-REV
tcctggtaactctcttctgcata

SEQ ID NO: 330 - DNA, 22 bp, Artificial Sequence
  hIFIT2-RT-FWD
ttattggtggcagaagaggaag

SEQ ID NO: 331 - DNA, 20 bp, Artificial Sequence
  hIFIT2-RT-REV
cctccatcaagttccaggtg

SEQ ID NO: 332 - DNA, 20 bp, Artificial Sequence
  hcGAS-FWD
cacgtatgtacccagaaccc

SEQ ID NO: 333 - DNA, 21 bp, Artificial Sequence
  hcGAS-REV
gtcctgaggcactgaagaaag

SEQ ID NO: 334 - DNA, 18 bp, Artificial Sequence
  mRsad2-FWD
ctgtgcgctggaaggttt

SEQ ID NO: 335 - DNA, 20 bp, Artificial Sequence
  mRsad2-REV
attcaggcaccaaacaggac

SEQ ID NO: 336 - DNA, 20 bp, Artificial Sequence
  mRn18s-FWD
gtaacccgttgaaccccatt

SEQ ID NO: 337 - DNA, 20 bp, Artificial Sequence
  mRn18s-REV
ccatccaatcggtagtagcg

SEQ ID NO: 338 - DNA, 20 bp, Artificial Sequence
  mIfih1-FWD
tcttggacacttgcttcgag

SEQ ID NO: 339 - DNA, 21 bp, Artificial Sequence
  mIfih1-REV
tccttctgcacaatccttctc

SEQ ID NO: 340 - DNA, 20 bp, Artificial Sequence
  mIfit1-RT-FWD
gagagtcaaggcaggtttct

SEQ ID NO: 341 - DNA, 23 bp, Artificial Sequence
  mIfit1-RT-REV
tctcacttccaaatcaggtatgt

SEQ ID NO: 342 - DNA, 20 bp, Artificial Sequence
  mOAS3-FWD
gtaccaccaggtgcagacac

SEQ ID NO: 343 - DNA, 20 bp, Artificial Sequence
  mOAS3-REV
gccatagttttccgtccaga

SEQ ID NO: 344 - RNA, 6 nt, Artificial Sequence
  Synthetic construct
rgucbc

SEQ ID NO: 345 - RNA, 21 nt, Artificial Sequence
  Synthetic construct
uaauuggcgucuggccuucuu

SEQ ID NO: 346 - RNA, 4 nt, Artificial Sequence
  Synthetic construct
acuu

SEQ ID NO: 347 - RNA, 6 nt, Artificial Sequence
  Synthetic construct
cacuuu

SEQ ID NO: 348 - RNA, 5 nt, Artificial Sequence
  Synthetic construct
uuuuc

SEQ ID NO: 349 - RNA, 5 nt, Artificial Sequence
  Synthetic construct
cguuu

SEQ ID NO: 350 - DNA, 4 bp, Artificial Sequence
  Synthetic construct
ggta

SEQ ID NO: 351 - DNA, 4 bp, Artificial Sequence
  Synthetic construct
cgct

SEQ ID NO: 352 - DNA, 4 bp, Artificial Sequence
  Synthetic construct
ccct

SEQ ID NO: 353 - DNA, 20 bp, Artificial Sequence
  C2-Mut1-PS
gcggtatccatgtcccaggc

SEQ ID NO: 354 - DNA, 20 bp, Artificial Sequence
  dC20
cccccccccccccccccccc

SEQ ID NO: 355 - DNA, 24 bp, Artificial Sequence
  A151
ttagggttagggttagggttaggg

SEQ ID NO: 356 - DNA, 24 bp, Artificial Sequence
  C151
ttcaaattcaaattcaaattcaaa

SEQ ID NO: 357 - DNA, 20 bp, Artificial Sequence
  IRS661
tgcttgcaagcttgcaagca

SEQ ID NO: 358 - DNA, 24 bp, Artificial Sequence
  IRS957
tgcttgacatcctggaggggttgt

SEQ ID NO: 359 - DNA, 24 bp, Artificial Sequence
  ODN 2006
tcgtcgttttgtcgttttgtcgtt

SEQ ID NO: 360 - DNA, 20 bp, Artificial Sequence
  ODN 1826
tccatgacgttcctgacgtt

SEQ ID NO: 361 - DNA, 45 bp, Artificial Sequence
  ISD45-FWD
tacagatctactagtgatctatgactgatctgtacatgatctaca

SEQ ID NO: 362 - DNA, 45 bp, Artificial Sequence
  ISD45-REV
tgtagatcatgtacagatcagtcatagatcactagtagatctgta

SEQ ID NO: 363 - DNA, 70 bp, Artificial Sequence
  ISD70-FWD
ccatcagaaagaggtttaatatttttgtgagaccatcgaagagagaaagagataaaacttttttacgact

SEQ ID NO: 364 - DNA, 70 bp, Artificial Sequence
  ISD70-REV
agtcgtaaaaaagttttatctctttctctcttcgatggtctcacaaaaatattaaacctctttctgatgg

SEQ ID NO: 365 - DNA, 3 bp, Artificial Sequence
  Synthetic construct
aaa

SEQ ID NO: 366 - DNA, 3 bp, Artificial Sequence
  Synthetic construct
aga

SEQ ID NO: 367 - DNA, 3 bp, Artificial Sequence
  Synthetic construct
caa

SEQ ID NO: 368 - DNA, 3 bp, Artificial Sequence
  Synthetic construct
cga

SEQ ID NO: 369 - DNA, 3 bp, Artificial Sequence
  Synthetic construct
gaa

SEQ ID NO: 370 - DNA, 3 bp, Artificial Sequence
  Synthetic construct
gga

SEQ ID NO: 371 - DNA, 3 bp, Artificial Sequence
  Synthetic construct
taa

SEQ ID NO: 372 - DNA, 3 bp, Artificial Sequence
  Synthetic construct
tga

SEQ ID NO: 373 - DNA, 3 bp, Artificial Sequence
  Synthetic construct
aac

SEQ ID NO: 374 - DNA, 3 bp, Artificial Sequence
  Synthetic construct
agc

SEQ ID NO: 375 - DNA, 3 bp, Artificial Sequence
  Synthetic construct
cgc

SEQ ID NO: 376 - DNA, 3 bp, Artificial Sequence
  Synthetic construct
gac

SEQ ID NO: 377 - DNA, 3 bp, Artificial Sequence
  Synthetic construct
ggc

SEQ ID NO: 378 - DNA, 3 bp, Artificial Sequence
  Synthetic construct
tac

SEQ ID NO: 379 - DNA, 3 bp, Artificial Sequence
  Synthetic construct
tgc

SEQ ID NO: 380 - DNA, 3 bp, Artificial Sequence
  Synthetic construct
aag

SEQ ID NO: 381 - DNA, 3 bp, Artificial Sequence
  Synthetic construct
agg

SEQ ID NO: 382 - DNA, 3 bp, Artificial Sequence
  Synthetic construct
cag

SEQ ID NO: 383 - DNA, 3 bp, Artificial Sequence
  Synthetic construct
cgg

SEQ ID NO: 384 - DNA, 3 bp, Artificial Sequence
  Synthetic construct
gag

SEQ ID NO: 385 - DNA, 3 bp, Artificial Sequence
  Synthetic construct
ggg

SEQ ID NO: 386 - DNA, 3 bp, Artificial Sequence
  Synthetic construct
tag

SEQ ID NO: 387 - DNA, 3 bp, Artificial Sequence
  Synthetic construct
tgg

SEQ ID NO: 388 - DNA, 3 bp, Artificial Sequence
  Synthetic construct
aat

SEQ ID NO: 389 - DNA, 3 bp, Artificial Sequence
  Synthetic construct
agt

SEQ ID NO: 390 - DNA, 3 bp, Artificial Sequence
  Synthetic construct
cat

SEQ ID NO: 391 - DNA, 3 bp, Artificial Sequence
  Synthetic construct
cgt

SEQ ID NO: 392 - DNA, 3 bp, Artificial Sequence
  Synthetic construct
gat

SEQ ID NO: 393 - DNA, 3 bp, Artificial Sequence
  Synthetic construct
ggt

SEQ ID NO: 394 - DNA, 3 bp, Artificial Sequence
  Synthetic construct
tat

SEQ ID NO: 395 - DNA, 3 bp, Artificial Sequence
  Synthetic construct
tgt

SEQ ID NO: 396 - DNA, 3 bp, Artificial Sequence
  Synthetic construct
ata

SEQ ID NO: 397 - DNA, 3 bp, Artificial Sequence
  Synthetic construct
cca

SEQ ID NO: 398 - DNA, 3 bp, Artificial Sequence
  Synthetic construct
cta

SEQ ID NO: 399 - DNA, 3 bp, Artificial Sequence
  Synthetic construct
gca

SEQ ID NO: 400 - DNA, 3 bp, Artificial Sequence
  Synthetic construct
gta

SEQ ID NO: 401 - DNA, 3 bp, Artificial Sequence
  Synthetic construct
tca

SEQ ID NO: 402 - DNA, 3 bp, Artificial Sequence
  Synthetic construct
tta

SEQ ID NO: 403 - DNA, 3 bp, Artificial Sequence
  Synthetic construct
acc

SEQ ID NO: 404 - DNA, 3 bp, Artificial Sequence
  Synthetic construct
atc

SEQ ID NO: 405 - DNA, 3 bp, Artificial Sequence
  Synthetic construct
ccc

SEQ ID NO: 406 - DNA, 3 bp, Artificial Sequence
  Synthetic construct
ctc

SEQ ID NO: 407 - DNA, 3 bp, Artificial Sequence
  Synthetic construct
gcc

SEQ ID NO: 408 - DNA, 3 bp, Artificial Sequence
  Synthetic construct
gtc

SEQ ID NO: 409 - DNA, 3 bp, Artificial Sequence
  Synthetic construct
tcc

SEQ ID NO: 410 - DNA, 3 bp, Artificial Sequence
  Synthetic construct
ttc

SEQ ID NO: 411 - DNA, 3 bp, Artificial Sequence
  Synthetic construct
acg

SEQ ID NO: 412 - DNA, 3 bp, Artificial Sequence
  Synthetic construct
atg

SEQ ID NO: 413 - DNA, 3 bp, Artificial Sequence
  Synthetic construct
ccg

SEQ ID NO: 414 - DNA, 3 bp, Artificial Sequence
  Synthetic construct
ctg

SEQ ID NO: 415 - DNA, 3 bp, Artificial Sequence
  Synthetic construct
gcg

SEQ ID NO: 416 - DNA, 3 bp, Artificial Sequence
  Synthetic construct
gtg

SEQ ID NO: 417 - DNA, 3 bp, Artificial Sequence
  Synthetic construct
tcg

SEQ ID NO: 418 - DNA, 3 bp, Artificial Sequence
  Synthetic construct
ttg

SEQ ID NO: 419 - DNA, 3 bp, Artificial Sequence
  Synthetic construct
act

SEQ ID NO: 420 - DNA, 3 bp, Artificial Sequence
  Synthetic construct
att

SEQ ID NO: 421 - DNA, 3 bp, Artificial Sequence
  Synthetic construct
cct

SEQ ID NO: 422 - DNA, 3 bp, Artificial Sequence
  Synthetic construct
gct

SEQ ID NO: 423 - DNA, 3 bp, Artificial Sequence
  Synthetic construct
gtt

SEQ ID NO: 424 - DNA, 3 bp, Artificial Sequence
  Synthetic construct
tct

SEQ ID NO: 425 - DNA, 3 bp, Artificial Sequence
  Synthetic construct
ttt

SEQ ID NO: 426 - RNA, 3 nt, Artificial Sequence
  Synthetic construct
aau

SEQ ID NO: 427 - RNA, 3 nt, Artificial Sequence
  Synthetic construct
acu

SEQ ID NO: 428 - RNA, 3 nt, Artificial Sequence
  Synthetic construct
cau

SEQ ID NO: 429 - RNA, 3 nt, Artificial Sequence
  Synthetic construct
agu

SEQ ID NO: 430 - RNA, 3 nt, Artificial Sequence
  Synthetic construct
aua

SEQ ID NO: 431 - RNA, 3 nt, Artificial Sequence
  Synthetic construct
auc

SEQ ID NO: 432 - RNA, 3 nt, Artificial Sequence
  Synthetic construct
aug

SEQ ID NO: 433 - RNA, 3 nt, Artificial Sequence
  Synthetic construct
auu

SEQ ID NO: 434 - RNA, 3 nt, Artificial Sequence
  Synthetic construct
cuc

SEQ ID NO: 435 - RNA, 3 nt, Artificial Sequence
  Synthetic construct
ccu

SEQ ID NO: 436 - RNA, 3 nt, Artificial Sequence
  Synthetic construct
cgu

SEQ ID NO: 437 - RNA, 3 nt, Artificial Sequence
  Synthetic construct
cua

SEQ ID NO: 438 - RNA, 3 nt, Artificial Sequence
  Synthetic construct
cug

SEQ ID NO: 439 - RNA, 3 nt, Artificial Sequence
  Synthetic construct
gcu

SEQ ID NO: 440 - RNA, 3 nt, Artificial Sequence
  Synthetic construct
gau

SEQ ID NO: 441 - RNA, 3 nt, Artificial Sequence
  Synthetic construct
uac

SEQ ID NO: 442 - RNA, 3 nt, Artificial Sequence
  Synthetic construct
guc

SEQ ID NO: 443 - RNA, 3 nt, Artificial Sequence
  Synthetic construct
gug

SEQ ID NO: 444 - RNA, 3 nt, Artificial Sequence
  Synthetic construct
guu

SEQ ID NO: 445 - RNA, 3 nt, Artificial Sequence
  Synthetic construct
uaa

SEQ ID NO: 446 - RNA, 3 nt, Artificial Sequence
  Synthetic construct
uag

SEQ ID NO: 447 - RNA, 3 nt, Artificial Sequence
  Synthetic construct
ugu

SEQ ID NO: 448 - RNA, 3 nt, Artificial Sequence
  Synthetic construct
uau

SEQ ID NO: 449 - RNA, 3 nt, Artificial Sequence
  Synthetic construct
uca

SEQ ID NO: 450 - RNA, 3 nt, Artificial Sequence
  Synthetic construct
ucc

SEQ ID NO: 451 - RNA, 3 nt, Artificial Sequence
  Short660v1-3-b
ucg

SEQ ID NO: 452 - RNA, 3 nt, Artificial Sequence
  Synthetic construct
ucu

SEQ ID NO: 453 - RNA, 3 nt, Artificial Sequence
  Synthetic construct
uga

SEQ ID NO: 454 - RNA, 3 nt, Artificial Sequence
  Synthetic construct
ugc

SEQ ID NO: 455 - RNA, 3 nt, Artificial Sequence
  Synthetic construct
ugg

SEQ ID NO: 456 - RNA, 3 nt, Artificial Sequence
  Synthetic construct
uua

SEQ ID NO: 457 - RNA, 3 nt, Artificial Sequence
  Synthetic construct
uuc

SEQ ID NO: 458 - RNA, 3 nt, Artificial Sequence
  Synthetic construct
uug

SEQ ID NO: 459 - RNA, 3 nt, Artificial Sequence
  Synthetic construct
uuu

SEQ ID NO: 460 - DNA, 20 bp, Artificial Sequence
  [C2]Mut1-v3-dC
cguttccccccccccccccc

SEQ ID NO: 461 - DNA, 20 bp, Artificial Sequence
  [HPRT]661
acuucgtggggtcctuuuca

SEQ ID NO: 462 - DNA, 20 bp, Artificial Sequence
  [HPRT]664
aacacttcgtggggtccuuu

SEQ ID NO: 463 - DNA, 20 bp, Artificial Sequence
  [HPRT]665
caacacttcgtgggguccuu

SEQ ID NO: 464 - RNA, 20 nt, Artificial Sequence
  [CDKN2B-AS1]2198
aagguggccacaggcaacgu

SEQ ID NO: 465 - DNA, 20 bp, Artificial Sequence
  [CDKN2B-AS1]2231
gucuccatgtcccaggccuc

SEQ ID NO: 466 - DNA, 20 bp, Artificial Sequence
  [CDKN2B-AS1]613
cagugtttttaatttuguag

SEQ ID NO: 467 - DNA, 20 bp, Artificial Sequence
  [CDKN2B-AS1]79
aaaauaaggggaatagggga

SEQ ID NO: 468 - DNA, 20 bp, Artificial Sequence
  [LINC-PINT]109
cucuctggtcccatcccuuc

SEQ ID NO: 469 - DNA, 20 bp, Artificial Sequence
  [LINC-PINT]2755
cuuuatattacaaagcuacu

SEQ ID NO: 470 - DNA, 20 bp, Artificial Sequence
  [LINC-PINT]2990
cacugtattttattacagaa

SEQ ID NO: 471 - DNA, 20 bp, Artificial Sequence
  [LINC-PINT]877 KL
cccuaatgctttcctcucca

SEQ ID NO: 472 - DNA, 20 bp, Artificial Sequence
  [LINC-PINT]94
ccuuctgctgccaagcccca

SEQ ID NO: 473 - DNA, 20 bp, Artificial Sequence
  [LINC-PINT]98
caucccttctgctgccaagc

SEQ ID NO: 474 - DNA, 20 bp, Artificial Sequence
  [LINC-PINT]99
ccaucccttctgctgccaag

SEQ ID NO: 475 - DNA, 20 bp, Artificial Sequence
  F5-EGFR-1014-MOE-Mut
tgtccttgcacgtgcctaag

SEQ ID NO: 476 - DNA, 20 bp, Artificial Sequence
  B3-CTNN2B-916-MOE-Mut
gggaaacgctatgcaaggtc

SEQ ID NO: 477 - DNA, 20 bp, Artificial Sequence
  F10-MB21D1-616-MOE-Mut
ggaccctcgaggccccaggc

SEQ ID NO: 478 - DNA, 16 bp, Artificial Sequence
  D2-CTNN2B-1642-LNA-Mut
gcccgctcagtgatgt

SEQ ID NO: 479 - RNA, 5 nt, Artificial Sequence
  Short660v1
cuucg

SEQ ID NO: 480 - DNA, 20 bp, Artificial Sequence
  HPRT-2OMe-847
auaggactccagatguuucc

SEQ ID NO: 481 - DNA, 16 bp, Artificial Sequence
  HPRT LNA-332 V1
tgatggcctcccatct

SEQ ID NO: 482 - DNA, 16 bp, Artificial Sequence
  CAMSAP1-1635 LNA KL
accacgtctgctctaa

SEQ ID NO: 483 - DNA, 16 bp, Artificial Sequence
  MB21D1-689 LNA KL
aacccctttcaccatc

SEQ ID NO: 484 - DNA, 16 bp, Artificial Sequence
  HPRT LNA-333 V1
gtgatggcctcccatc

SEQ ID NO: 485 - DNA, 16 bp, Artificial Sequence
  HPRT LNA-335 V1
atgtgatggcctccca

SEQ ID NO: 486 - DNA, 16 bp, Artificial Sequence
  CTNN2B-808 LNA V1
tgtctggaagcttcct

SEQ ID NO: 487 - DNA, 16 bp, Artificial Sequence
  EGFR-3652 LNA KL
agtgttgagatactcg

SEQ ID NO: 488 - DNA, 16 bp, Artificial Sequence
  CAMSAP1-1739 LNA KL
tactttccgaggtgtc

SEQ ID NO: 489 - DNA, 16 bp, Artificial Sequence
  CAMSAP1-5594 LNA V1
ggctcggcttgcctac

SEQ ID NO: 490 - DNA, 16 bp, Artificial Sequence
  MB21D1-719 LNA KL
gcacttcagtctgagc

SEQ ID NO: 491 - DNA, 16 bp, Artificial Sequence
  HPRT LNA-667 V1
caacacttcgtggggt

SEQ ID NO: 492 - DNA, 16 bp, Artificial Sequence
  EGFR-3908 LNA KL
gtatcgaaagagtctg

SEQ ID NO: 493 - DNA, 16 bp, Artificial Sequence
  EGFR-2874 LNA V1
gccatccacttgatag

SEQ ID NO: 494 - DNA, 16 bp, Artificial Sequence
  MB21D1-522 LNA V1
gtcttggcttcgtgga

SEQ ID NO: 495 - DNA, 16 bp, Artificial Sequence
  CAMSAP1-4697 LNA KL
ttttggctgggatcaa

SEQ ID NO: 496 - DNA, 16 bp, Artificial Sequence
  CAMSAP1-2584 LNA V1
gccatgctggctcgga

SEQ ID NO: 497 - DNA, 16 bp, Artificial Sequence
  HPRT LNA-551 KL
tccacaatcaagacat

SEQ ID NO: 498 - DNA, 16 bp, Artificial Sequence
  HPRT LNA-666 V1
aacacttcgtggggtc

SEQ ID NO: 499 - DNA, 16 bp, Artificial Sequence
  EGFR-1013 LNA V1
ccttgcacgtggcttc

SEQ ID NO: 500 - DNA, 16 bp, Artificial Sequence
  CAMSAP1-6273 LNA KL
aaaggactgaggaaag

SEQ ID NO: 501 - DNA, 16 bp, Artificial Sequence
  CAMSAP1-2709 LNA V1
ttcaggcgctgccttg

SEQ ID NO: 502 - DNA, 16 bp, Artificial Sequence
  MB21D1-1483 LNA KL
tgactgtcttgagggt

SEQ ID NO: 503 - DNA, 16 bp, Artificial Sequence
  MB21D1-520 LNA V1
cttggcttcgtggagc

SEQ ID NO: 504 - DNA, 16 bp, Artificial Sequence
  HPRT LNA-692 KL
aatccaacaaagtctg

SEQ ID NO: 505 - DNA, 16 bp, Artificial Sequence
  HPRT LNA-331 V1
gatggcctcccatctc

SEQ ID NO: 506 - DNA, 16 bp, Artificial Sequence
  EGFR-1014 LNA V1
tccttgcacgtggctt

SEQ ID NO: 507 - DNA, 16 bp, Artificial Sequence
  EGFR-1016 LNA V1
tgtccttgcacgtggc

SEQ ID NO: 508 - DNA, 16 bp, Artificial Sequence
  MB21D1-521 LNA V1
tcttggcttcgtggag

SEQ ID NO: 509 - DNA, 16 bp, Artificial Sequence
  HPRT LNA-732 KL
gtcaagggcatatcct

SEQ ID NO: 510 - DNA, 16 bp, Artificial Sequence
  CTNN2B-2446 LNA V1
tccatacccaaggcat

SEQ ID NO: 511 - DNA, 16 bp, Artificial Sequence
  HPRT LNA-668 V1
ccaacacttcgtgggg

SEQ ID NO: 512 - DNA, 16 bp, Artificial Sequence
  CTNN2B-2479 LNA V1
tggtggccacccatct

SEQ ID NO: 513 - DNA, 16 bp, Artificial Sequence
  EGFR-3266 LNA V1
catccaccacgtcgtc

SEQ ID NO: 514 - DNA, 16 bp, Artificial Sequence
  EGFR-727 LNA V1
gtcccgccactggatg

SEQ ID NO: 515 - DNA, 16 bp, Artificial Sequence
  CAMSAP1-2113 LNA V1
gtcttcatcggccctg

SEQ ID NO: 516 - DNA, 16 bp, Artificial Sequence
  MB21D1-697 LNA V1
tggtccacaacccctt

SEQ ID NO: 517 - DNA, 16 bp, Artificial Sequence
  HPRT LNA-1027 KL
tccgcccaaagggaac

SEQ ID NO: 518 - DNA, 16 bp, Artificial Sequence
  HPRT LNA-326 V1
cctcccatctccttca

SEQ ID NO: 519 - DNA, 16 bp, Artificial Sequence
  EGFR-2859 LNA V1
ggcactttgcctcctt

SEQ ID NO: 520 - DNA, 16 bp, Artificial Sequence
  MB21D1-526 LNA V1
ggaggtcttggcttcg

SEQ ID NO: 521 - DNA, 16 bp, Artificial Sequence
  HPRT LNA-1353 KL
tgacaaagattcactg

SEQ ID NO: 522 - DNA, 20 bp, Artificial Sequence
  HPRT-489 MOE
atgtcccctgttgactggtc

SEQ ID NO: 523 - DNA, 20 bp, Artificial Sequence
  CTNN2B-311 MOE
gccgcttttctgtctggttc

SEQ ID NO: 524 - DNA, 20 bp, Artificial Sequence
  CTNN2B-1575 MOE
acccacttggcagaccatca

SEQ ID NO: 525 - DNA, 20 bp, Artificial Sequence
  EGFR-1225 MOE
caaggcccttcgcacttctt

SEQ ID NO: 526 - DNA, 20 bp, Artificial Sequence
  CAMSAP1-1635 MOE
gaaccacgtctgctctaaca

SEQ ID NO: 527 - DNA, 20 bp, Artificial Sequence
  CAMSAP1-4915 MOE
ttgcacacttcgtacccaaa

SEQ ID NO: 528 - DNA, 20 bp, Artificial Sequence
  MB21D1-689 MOE
acaacccctttcaccatccc

SEQ ID NO: 529 - DNA, 20 bp, Artificial Sequence
  HPRT-732 MOE
tagtcaagggcatatcctac

SEQ ID NO: 530 - DNA, 20 bp, Artificial Sequence
  MB21D1-523 MOE
gaggtcttggcttcgtggag

SEQ ID NO: 531 - DNA, 20 bp, Artificial Sequence
  CTNN2B-808 MOE
cgtgtctggaagcttccttt

SEQ ID NO: 532 - DNA, 20 bp, Artificial Sequence
  EGFR-1015 MOE
gtgtccttgcacgtggcttc

SEQ ID NO: 533 - DNA, 20 bp, Artificial Sequence
  CAMSAP1-1739 MOE
cttactttccgaggtgtcca

SEQ ID NO: 534 - DNA, 20 bp, Artificial Sequence
  HPRT-1027 MOE
aatccgcccaaagggaactg

SEQ ID NO: 535 - DNA, 20 bp, Artificial Sequence
  CTNN2B-1294 MOE
cagatagcaccttcagcact

SEQ ID NO: 536 - DNA, 20 bp, Artificial Sequence
  EGFR-2874 MOE
atgccatccacttgataggc

SEQ ID NO: 537 - DNA, 20 bp, Artificial Sequence
  CTNN2B-1642 MOE
tggcaggctcagtgatgtct

SEQ ID NO: 538 - DNA, 20 bp, Artificial Sequence
  CAMSAP1-2584 MOE
ttgccatgctggctcggact

SEQ ID NO: 539 - DNA, 20 bp, Artificial Sequence
  CTNN2B-3648 MOE
ctcttgaagcatcgtatcac

SEQ ID NO: 540 - DNA, 20 bp, Artificial Sequence
  CTNN2B-2516 MOE
agatctggcagcccatcaac

SEQ ID NO: 541 - DNA, 20 bp, Artificial Sequence
  CAMSAP1-2709 MOE
gcttcaggcgctgccttgcc

SEQ ID NO: 542 - DNA, 20 bp, Artificial Sequence
  HPRT-333 MOE
atgtgatggcctcccatctc

SEQ ID NO: 543 - DNA, 20 bp, Artificial Sequence
  HPRT-335 MOE
caatgtgatggcctcccatc

SEQ ID NO: 544 - DNA, 20 bp, Artificial Sequence
  CAMSAP1-2112 MOE
tgtcttcatcggccctgcct

SEQ ID NO: 545 - DNA, 20 bp, Artificial Sequence
  HPRT-667 MOE
tccaacacttcgtggggtcc

SEQ ID NO: 546 - DNA, 20 bp, Artificial Sequence
  CTNN2B-2479 MOE
ggtggtggccacccatctca

SEQ ID NO: 547 - DNA, 20 bp, Artificial Sequence
  EGFR-727 MOE
atgtcccgccactggatgct

SEQ ID NO: 548 - DNA, 20 bp, Artificial Sequence
  HPRT-329 MOE
gatggcctcccatctccttc

SEQ ID NO: 549 - DNA, 20 bp, Artificial Sequence
  CTNN2B-1576 MOE
cacccacttggcagaccatc

SEQ ID NO: 550 - DNA, 20 bp, Artificial Sequence
  EGFR-2859 MOE
taggcactttgcctccttct

SEQ ID NO: 551 - DNA, 20 bp, Artificial Sequence
  CAMSAP1-1850 MOE
gatgctcctcggtctccctt

SEQ ID NO: 552 - DNA, 19 bp, Artificial Sequence
  ASO2
tccggcctcggaagctctc

SEQ ID NO: 553 - DNA, 20 bp, Artificial Sequence
  C4
ccatgtcccaggcctccagt

SEQ ID NO: 554 - DNA, 18 bp, Artificial Sequence
  C11
ttggcctgtggatgcttt

SEQ ID NO: 555 - DNA, 20 bp, Artificial Sequence
  A11
aaatgtcctggccctcactg

SEQ ID NO: 556 - DNA, 19 bp, Artificial Sequence
  ASO-2
ccggcctcggaagcucucu

SEQ ID NO: 557 - DNA, 20 bp, Artificial Sequence
  196861965-C2
gcagucuccaugtcccaggc

SEQ ID NO: 558 - DNA, 20 bp, Artificial Sequence
  195513083-E3
gauggttccagucccucuuc

SEQ ID NO: 559 - DNA, 19 bp, Artificial Sequence
  P2-F2
agcagucuccatgtcccag

SEQ ID NO: 560 - DNA, 18 bp, Artificial Sequence
  P2-C2
gcagucuccatgtcccag

SEQ ID NO: 561 - DNA, 17 bp, Artificial Sequence
  P2-E8
gggucucctccacaccc

SEQ ID NO: 562 - DNA, 21 bp, Artificial Sequence
  P2-E10
gauggttccagucccucuuuc

SEQ ID NO: 563 - DNA, 16 bp, Artificial Sequence
  P2-F9
ggucccatcccttctg

SEQ ID NO: 564 - DNA, 19 bp, Artificial Sequence
  P2-A6
gattaaacagattaataca

SEQ ID NO: 565 - DNA, 20 bp, Artificial Sequence
  P2-C6
ggattaaaacagattaatac

SEQ ID NO: 566 - DNA, 20 bp, Artificial Sequence
  P2-E6
cttgtgaaaagattatcttc

SEQ ID NO: 567 - DNA, 20 bp, Artificial Sequence
  P2-C5
cctagaaagaagcaaagatt

SEQ ID NO: 568 - DNA, 20 bp, Artificial Sequence
  P2-D6
aatttaaagcatgaatatta

SEQ ID NO: 569 - DNA, 19 bp, Artificial Sequence
  P2-D10
cacccuucuctctgguccc

SEQ ID NO: 570 - DNA, 16 bp, Artificial Sequence
  P2-A8
ccuucuctctggtccc

SEQ ID NO: 571 - DNA, 16 bp, Artificial Sequence
  P2-A9
uggtcccatcccuucu

SEQ ID NO: 572 - DNA, 15 bp, Artificial Sequence
  P2-A3
ctgctgccaccuucu

SEQ ID NO: 573 - DNA, 20 bp, Artificial Sequence
  P2-H9
gucccatcccuucugcugcc

SEQ ID NO: 574 - DNA, 17 bp, Artificial Sequence
  P2-H8
ucctccacacccuucuc

SEQ ID NO: 575 - DNA, 17 bp, Artificial Sequence
  P2-H2
gcctccagtgucuucuc

SEQ ID NO: 576 - RNA, 19 nt, Artificial Sequence
  P2-G7
ucccaacucuucuaacugu

SEQ ID NO: 577 - DNA, 16 bp, Artificial Sequence
  G5
ggcatccaccacgtcg

SEQ ID NO: 578 - DNA, 18 bp, Artificial Sequence
  E6
ccttgcacgtggcttcgt

SEQ ID NO: 579 - DNA, 17 bp, Artificial Sequence
  F5
ccttgcacgtggcttcg

SEQ ID NO: 580 - DNA, 16 bp, Artificial Sequence
  E5
atttcagagcagcttc

SEQ ID NO: 581 - DNA, 19 bp, Artificial Sequence
  G8
gtccacatcctgtggctcg

SEQ ID NO: 582 - RNA, 20 nt, Artificial Sequence
  C2Mut1-dC-2
gguacccccccccccccccc

SEQ ID NO: 583 - RNA, 20 nt, Artificial Sequence
  C2Mut1-dC-3
gguccccccccccccccccc

SEQ ID NO: 584 - DNA, 17 bp, Artificial Sequence
  E9
gactgtcttgagggttc

SEQ ID NO: 585 - DNA, 19 bp, Artificial Sequence
  F3
cgtgtctggaagcttcctt

SEQ ID NO: 586 - DNA, 18 bp, Artificial Sequence
  D10
cggcctcggaagctctct

SEQ ID NO: 587 - DNA, 19 bp, Artificial Sequence
  HPRT-664 MOE
aacattcgtggggtccttt

SEQ ID NO: 588 - DNA, 18 bp, Artificial Sequence
  Synthetic construct
uccggcctcggaagctct

SEQ ID NO: 589 - DNA, 22 bp, Artificial Sequence
  Synthetic construct
cuucggcctcggaagctctuuc

SEQ ID NO: 590 - DNA, 20 bp, Artificial Sequence
  Synthetic construct
gaacgtggggtccttuucac

SEQ ID NO: 591 - DNA, 3 bp, Artificial Sequence
  Synthetic construct
  misc_feature (3)..(3): n is a, c, g, t or u
gan

SEQ ID NO: 592 - DNA, 3 bp, Artificial Sequence
  Synthetic construct
  misc_feature (3)..(3): n is a, c, g, t or u
gun

SEQ ID NO: 593 - DNA, 6 bp, Artificial Sequence
  Synthetic construct
ggctcc

SEQ ID NO: 594 - DNA, 15 bp, Artificial Sequence
  Synthetic construct
gguatcccccccccc

SEQ ID NO: 595 - DNA, 9 bp, Artificial Sequence
  Synthetic construct
gguatcccc

SEQ ID NO: 596 - DNA, 5 bp, Artificial Sequence
  Synthetic construct
ugutt

SEQ ID NO: 597 - DNA, 5 bp, Artificial Sequence
  Synthetic construct
cgutt

SEQ ID NO: 598 - DNA, 3 bp, Artificial Sequence
  Synthetic construct
  misc_feature (3)..(3): n is a, c, g, t or u
ttn

SEQ ID NO: 599 - DNA, 3 bp, Artificial Sequence
  Synthetic construct
  misc_feature (3)..(3): n is a, c, g, t or u
uun